(19) United States
(12) Patent Application Publication
(10) Pub. No.: US 2025/0101038 A1
(43) Pub. Date: Mar. 27, 2025

(54) COMPOUNDS

(71) Applicant: Psylo Pty Ltd, Sydney, New South Wales (AU)

(72) Inventors: Samuel Banister, Sydney NSW (AU); William Jorgensen, Sydney NSW (AU); Jinlong Tan, Sydney NSW (AU)

(21) Appl. No.: 18/723,719

(22) PCT Filed: Dec. 23, 2022
(86) PCT No.: PCT/AU2022/051592

§ 371 Date: Jun. 24, 2024

(30) Foreign Application Priority Data
Dec. 24, 2021 (AU) 2021904274

(51) Int. Cl. C07D 498/04; A61K 31/4045; A61K 31/423; A61P 25/24; C07D 209/16; C07D 491/048

(52) U.S. Cl. CPC C07D 498/04; A61K 31/4045; A61K 31/423; A61P 25/24; C07D 209/16; C07D 491/048

(57) ABSTRACT

The present disclosure relates generally to compounds, their methods of synthesis, and their use in the treatment of mental illness or central nervous system disorders.

This application claims priority to Australian provisional application no. 2021904274 (filed 24 Dec. 2021), which is entirely incorporated herein by reference.

### FIELD OF THE INVENTION

The present disclosure relates generally to novel compounds, their methods of synthesis, and their use in the treatment of mental illness or central nervous system disorders.

### BACKGROUND OF THE INVENTION

Mental illness covers many neuropsychiatric disorders which cause enormous burden on the lives of their sufferers. Diagnoses such as treatment resistant depression, major depressive disorder, eating disorders, substance abuse disorders, post-traumatic stress disorder, obsessive compulsive disorder, attention deficit disorders, schizophrenia, and others can cause such devastating symptoms that many sufferers lose the capability of leading a normal life.

A variety of serotonergic drugs such as antidepressants, serotonin reuptake inhibitors, monoamine oxidase inhibitors, selective serotonin reuptake inhibitors, and others are commercially available to treat mental illnesses. Unfortunately, in many indications, these therapeutics provide limited benefit when compared to a placebo. Additionally, these therapeutics can result in a wide range of side effects including loss of libido, insomnia, fatigue, weight gain, and others. In spite of their limited efficacy, these drugs continue to be used to treat neuropsychiatric conditions as well as a broad range of auxiliary medical indications. There have been limited advances in new treatment options since many of these drugs were released, and the pharmaceutical industry has come under increased financial pressure to de-emphasise neuroscience programmes entirely. The unmet need for more efficacious mental health treatment is on the rise, and the global COVID-19 pandemic is likely to increase disease burden around the world.

In the 1950s and 1960s, the use of psychedelic drugs to treat various mental illnesses was extensively explored, and these substances showed promise as treatments for many diseases of the central nervous system (CNS). Following decades of prohibition, scientific research into the application of psychedelics as treatments for mental illnesses has been gaining momentum. The serotonergic psychedelic agent psilocybin has been designated a Breakthrough Therapy by the FDA for the treatment of major depressive disorder (2019) and treatment-resistant depression (2018). Psilocybin is the prodrug compound produced by more than many species of mushrooms known collectively as psilocybin mushrooms or “magic mushrooms”. Psilocybin is rapidly metabolized to the bioactive compound psilocin, which produces a state of altered consciousness including changes in perception, visual hallucinations, and distorted sense of space, time, and self. Many patients report spiritual or “mystical” experiences which have profound and lasting impact on the patients' mood and behaviour. Psilocybin has shown promise in more than 50 clinical trials for neuropsychiatric indications, including numerous anxiety disorders, obsessive-compulsive disorder, anorexia nervosa, alcohol dependence, and tobacco addiction. Psilocybin and other psychedelic compounds such as N,N-dimethyltryptamine (DMT) and 5-methoxy-N,N-dimethyltryptamine (5-MeO-DMT) have both immediate and persistent effects on mental state, with the latter extending far beyond the duration of action, possibly as a result of their ability to incite increased neuroplasticity, promote neural outgrowth, and increase spine density of the synaptic neurons in the brain.

To date, psilocybin remains classified as a controlled substance and/or drug of abuse in most countries under national drug laws. However, clinical investigations have recently led to increased awareness of the potential for psychedelic drugs as breakthrough therapies to treat CNS diseases of enormous unmet medical need.

Despite its therapeutic potential, psilocybin and other psychedelics remain scheduled drugs of abuse in most countries and the commercial path to market for these drugs as medicines is uncertain. As an adjunct to psychotherapy, the long duration of action of psilocybin and LSD make treatment sessions costly and impractical for broad implementation. In spite of a long history of safe human use, several adverse events have been reported in clinical trials, and it is possible that these may be attributed to signalling bias at 5-HT2A (the primary target) or off-target activity at, for example, 5-HT2B receptors (a cardiac liability antitarget) or 5-HT1A (an anxiolytic target) or 5-HT2C receptors (a disease-relevant target for obesity and some genetic epilepsies, for example). Naturally-occurring psychedelics provide important lead structures for a new generation of neurotherapeutic agents with novel mechanisms of action and/or superior clinical efficacy to currently available neuropsychiatric medications.

In view of the foregoing there is an ongoing need to develop new compounds which may be useful in the treatment of mental illness or central nervous system disorders.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect the present disclosure provides a compound of formula (I):

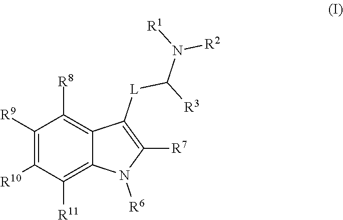

or a pharmaceutically acceptable salt, solvate, tautomer, N-oxide, stereoisomer, metabolite, polymorph and/or prodrug thereof,
wherein
R¹ and R² are each independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴ and SO₂R⁴,
said C₃₋₈ cycloalkyl, C₄₋₁₄ alkylenecycloalkyl, C₃-C₈ heterocycloalkyl, C₆₋₁₂ aryl, C₇₋₁₈ alkylenearyl, C₅₋₁₀ heteroaryl, and C₆₋₁₆ alkyleneheteroaryl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
alternatively R¹ and R² are combined with the atoms to which they are attached to form a C₃₋₈ heterocycloalkyl including 1 or 2 additional ring heteromoieties selected from O, S, S(O), SO₂, N and NR⁴,
said C₃₋₈ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆alkenyl, C₂₋₆ haloalkenyl, C₂₋₆ alkynyl, C₂₋₆ haloalkynyl, C₁₋₈ alkylamino, C₁₋₈ alkylsulfonyl, C₃₋₆ cycloalkyl and C₃₋₆ heterocycloalkyl including 1 or 2 ring heteromoieties selected from O, S, N, S(O), SO₂ and NR⁴;
R³ is selected from hydrogen, C₁₋₆ alkyl, C₃₋₈ cycloalkyl, or C₄₋₁₄ alkylenecycloalkyl;
alternatively R³ and one of R¹ and R² are combined with the atoms to which they are attached to form a C₃₋₁₂ heterocycloalkyl,
said C₃₋₁₂ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
each R⁴ is independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, and C₃₋₇ heterocycloalkyl including 1 or 2 ring heteromoieties selected from O, S, S(O), SO₂, N and NR⁵,
said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl and C₃₋₇ heterocycloalkyl each being optionally substituted with one or more substituents independently selected from halogen, CN, C₁₋₈ alkoxy, C₁₋₈ alkylamino, C₁₋₈ alkylsulfonyl, CO₂R⁵, C(O)N(R⁵)₂, OR⁵, N(R⁵)₂, NO₂, SR⁵ and SO₂R⁵,
said C₃-C₇ cycloalkyl and C₃₋₇ heterocycloalkyl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N and NR⁵;
each R⁵ is independently selected from hydrogen, C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl,
said C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
L is selected from C₁₋₄ alkylene, C₂-C₄ alkenylene and C₂-C₄ alkynylene;
R⁶ is selected from hydrogen, C₁₋₆ alkyl, C₂₋₆ alkenyl, C₂₋₆ alkynyl, C₁₋₆ alkyleneP(O)(OR¹²)₂, C(O)R¹², CO₂R¹², C(O)N(R¹²)₂, S(O) R¹² and SO₂R¹², C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂R¹², C(O)N(R¹²)₂, OR¹², N(R¹²)₂, NO₂, SR¹² and SO₂R¹²,
said C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹²;
each R¹² is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
R⁷, R⁸, R⁹, R¹⁰ and R¹¹ are each independently selected from hydrogen, halogen, CN, OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O) R¹³, OS(O)N(R¹³)₂, OSO₂R¹³, OP(O)(OR¹³)₂, OC₁₋₆alkyleneP(O)(OR¹³)₂, S(O) R¹³, S(O) N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
alternatively, R⁶ and R⁷ are combined with the atoms to which they are each attached to form a C₄₋₁₀ heterocycloalkyl or a C₅₋₁₀ heteroaryl,
said C₄₋₁₀ heterocycloalkyl and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
alternatively, R⁷ and one of R¹, R², or R³ are combined with the atoms to which they are attached to form a C₅₋₈ heterocycloalkyl,
said C₅₋₈ heterocyclyalkyl being further optionally substituted with one or more substituents selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
alternatively, R⁸ and R⁹, or R⁹ and R¹⁰, or R¹⁰ and R¹¹ are combined with the atoms to which they are each attached to form a C₄₋₈ cycloalkyl, C₅₋₈ heterocycloalkyl, C₆₋₁₂ aryl, or C₅₋₁₀ heteroaryl,
said C₄₋₈ cycloalkyl, C₅₋₈ heterocycloalkyl, C₆₋₁₂ aryl, and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
each R¹⁴ is independently selected from hydrogen, C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl,
said C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
wherein at least two or more of R⁷, R⁸, R⁹, R¹⁰ and R¹¹ are not hydrogen;
wherein:
when R¹ and R² are each methyl, R³ is hydrogen, R⁶ is selected from hydrogen, methyl, ethyl and propyl, and one of R⁹, R¹⁰ and R¹¹ is fluoro and the other of R⁹, R¹⁰ and R¹¹ are hydrogen, then R⁸ is not selected from OH, OCH₃, OCH₂CH₃, OCH₂CH₂CH₃ or OBn; and
when R¹ and R² are each methyl, R³ is hydrogen, R⁶ is selected from hydrogen, methyl, ethyl and propyl, R⁹ is fluoro, and R¹¹ is hydrogen, then R¹⁰ is not selected from OH, OCH₃, OCH₂CH₃, OCH₂CH₂CH₃ and OBn; and
wherein the compound is not selected from the following:

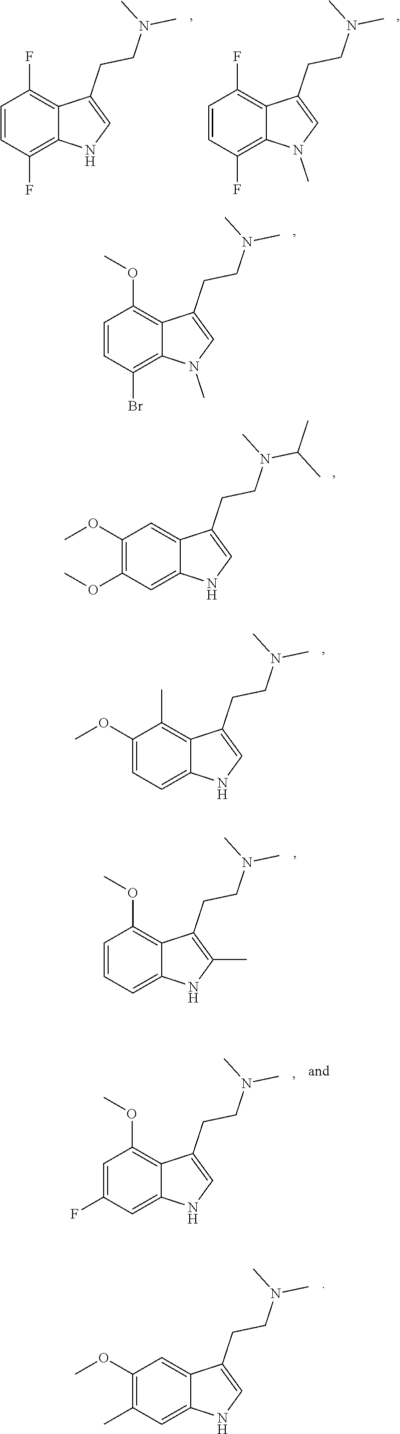

In another aspect of the present disclosure there is provided a medicament comprising a compound of formula (I) according to any one of the herein disclosed embodiments, or a pharmaceutically acceptable salt, solvate, tautomer, N-oxide, stereoisomer, metabolite, polymorph or prodrug thereof.

In another aspect of the present disclosure there is provided a pharmaceutical composition comprising a compound of formula (I) according to any one of the herein disclosed embodiments, or a pharmaceutically acceptable salt, solvate, tautomer, N-oxide, stereoisomer, metabolite, polymorph or prodrug thereof, and a pharmaceutically acceptable excipient.

In another aspect the present disclosure provides a pharmaceutical composition comprising a compound according to any one of the herein disclosed embodiments, or a pharmaceutically acceptable salt, solvate, tautomer, N-oxide, stereoisomer, metabolite, polymorph or prodrug thereof, an additional therapeutic agent, and a pharmaceutically acceptable excipient.

In another aspect of the present disclosure there is provided a method of treating a disease, disorder or condition by activation of a serotonin receptor, the method comprising administering to a subject in need thereof a compound of formula (I):

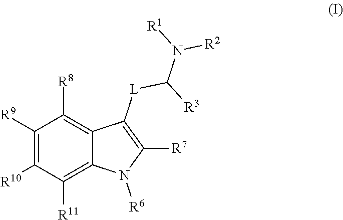

or a pharmaceutically acceptable salt, solvate, tautomer, N-oxide, stereoisomer, metabolite, polymorph or prodrug thereof,
wherein
R¹ and R² are each independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴ and SO₂R⁴,
said C₃₋₈ cycloalkyl, C₄₋₁₄ alkylenecycloalkyl, C₃-C₈ heterocycloalkyl, C₆₋₁₂ aryl, C₇₋₁₈ alkylenearyl, C₅₋₁₀ heteroaryl, and C₆₋₁₆ alkyleneheteroaryl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
alternatively R¹ and R² are combined with the atoms to which they are attached to form a C₃₋₈ heterocycloalkyl including 1 or 2 additional ring heteromoieties selected from O, S, S(O), SO₂, N and NR⁴,
said C₃₋₈ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆alkenyl, C₂₋₆ haloalkenyl, C₂₋₆ alkynyl, C₂₋₆ haloalkynyl, C₁₋₈ alkylamino, C₁₋₈ alkylsulfonyl, C₃₋₆ cycloalkyl and C₃₋₆ heterocycloalkyl including 1 or 2 ring heteromoieties selected from O, S, N, S(O), SO₂ and NR⁴;
R³ is selected from hydrogen, C₁₋₆ alkyl, C₃₋₈ cycloalkyl, or C₄₋₁₄ alkylenecycloalkyl;
alternatively R³ and one of R¹ and R² are combined with the atoms to which they are attached to form a C₃₋₁₂ heterocycloalkyl,
said C₃₋₁₂ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
each R⁴ is independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, and C₃₋₇ heterocycloalkyl including 1 or 2 ring heteromoieties selected from O, S, S(O), SO₂, N and NR⁵
said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl and C₃₋₇ heterocycloalkyl each being optionally substituted with one or more substituents independently selected from halogen, CN, C₁₋₈ alkoxy, C₁₋₈ alkylamino, C₁₋₈ alkylsulfonyl, CO₂R⁵, C(O)N(R⁵)₂, OR⁵, N(R⁵)₂, NO₂, SR⁵ and SO₂R⁵,
said C₃-C₇ cycloalkyl and C₃₋₇ heterocycloalkyl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N and NR⁵;
each R⁵ is independently selected from hydrogen, C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl,
said C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
L is selected from C₁₋₄ alkylene, C₂-C₄ alkenylene and C₂-C₄ alkynylene;
R⁶ is selected from hydrogen, C₁₋₆ alkyl, C₂₋₆ alkenyl, C₂₋₆ alkynyl, C₁₋₆ alkyleneP(O)(OR¹²)₂, C(O)R¹², CO₂R¹², C(O)N(R¹²)₂, S(O) R¹² and SO₂R¹², C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂R¹², C(O)N(R¹²)₂, OR¹², N(R¹²)₂, NO₂, SR¹² and SO₂R¹²;
said C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹²;
each R¹² is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
R⁷, R⁸, R⁹, R¹⁰ and R¹¹ are each independently selected from hydrogen, halogen, CN, OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O) R¹³, OS(O)N(R¹³)₂, OSO₂R¹³, OP(O)(OR¹³)₂, OC₁₋₆alkyleneP(O)(OR¹³)₂, S(O) R¹³, S(O) N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
alternatively, R⁶ and R⁷ are combined with the atoms to which they are each attached to form a C₄₋₁₀ heterocycloalkyl or a C₅₋₁₀ heteroaryl,
said C₄₋₁₀ heterocycloalkyl and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
alternatively, R⁷ and one of R¹, R², or R³ are combined with the atoms to which they are attached to form a C₅₋₈ heterocycloalkyl,
said C₅₋₈ heterocyclyalkyl being further optionally substituted with one or more substituents selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
alternatively, R⁸ and R⁹, or R⁹ and R¹⁰, or R¹⁰ and R¹¹ are combined with the atoms to which they are each attached to form a C₄₋₈ cycloalkyl, C₅₋₈ heterocycloalkyl, C₆₋₁₂ aryl, or C₅₋₁₀ heteroaryl,
said C₄₋₈ cycloalkyl, C₅₋₈ heterocycloalkyl, C₆₋₁₂ aryl, and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
each R¹⁴ is independently selected from hydrogen, C₁₋₆ alkyl, C₂-C₆ alkenyl, C₂-C₆ alkynyl, C₁-C₆ haloalkyl, C₃-C₇ cycloalkyl, C₃₋₁₀ heterocycloalkyl, C₆₋₁₂ aryl and C₅₋₁₀ heteroaryl,
said C₁₋₆ alkyl, C₂-C₆ alkenyl, C₂-C₆ alkynyl, C₁-C₆ haloalkyl, C₃-C₇ cycloalkyl, C₃₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
wherein at least two or more of R⁷, R⁸, R⁹, R¹⁰ and R¹¹ are not hydrogen;
wherein:
when R¹ and R² are each methyl, R³ is hydrogen, R⁶ is selected from hydrogen, methyl, ethyl and propyl, and one of R⁹, R¹⁰ and R¹¹ is fluoro and the other of R⁹, R¹⁰ and R¹¹ are hydrogen, then R⁸ is not selected from OH, OCH₃, OCH₂CH₃, OCH₂CH₂CH₃ or OBn; and
when R¹ and R² are each methyl, R³ is hydrogen, R⁶ is selected from hydrogen, methyl, ethyl and propyl, R⁹ is fluoro, and R¹¹ is hydrogen, then R¹⁰ is not selected from OH, OCH₃, OCH₂CH₃, OCH₂CH₂CH₃ and OBn; and
wherein the compound is not selected from the following:

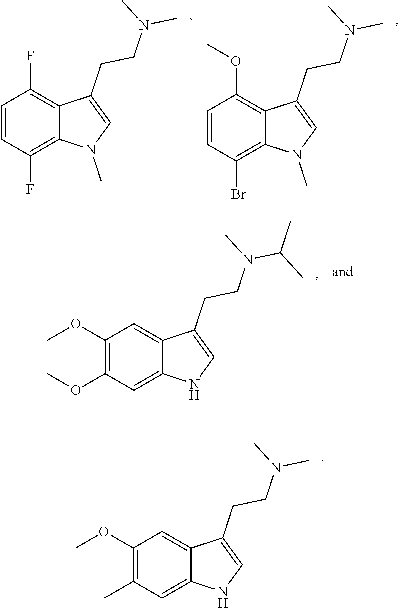

In some embodiments, the compound is not selected from the following:

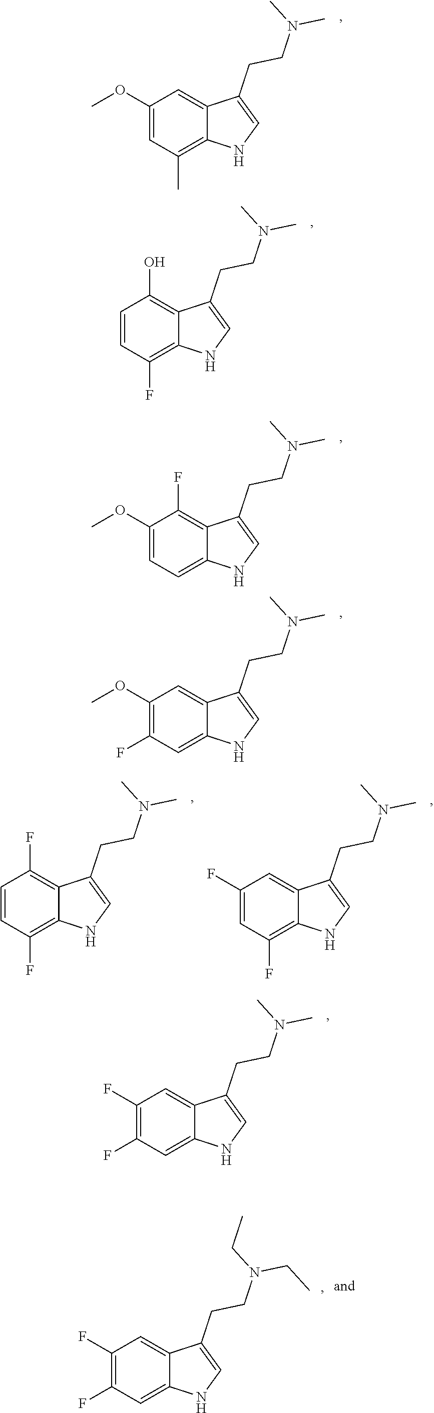

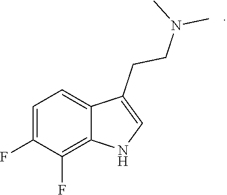 [SMILES: CN(C)CCc1c[nH]c2c(F)c(F)ccc12]

In some embodiments, the compound is not selected from the following:

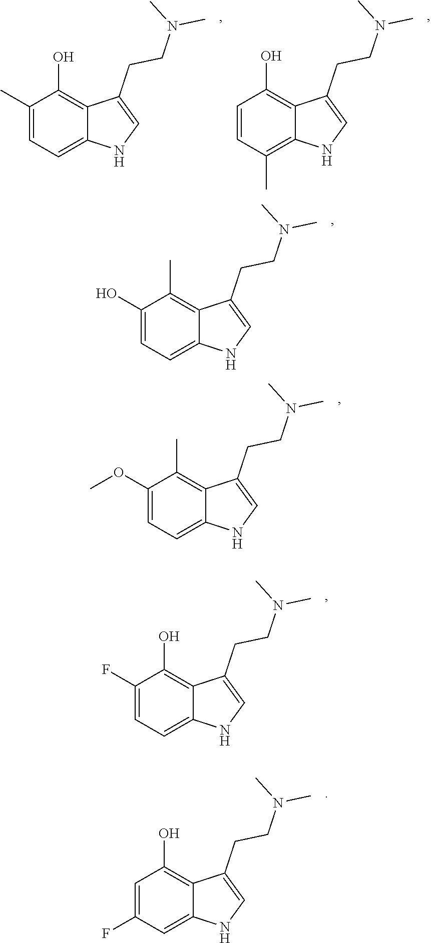

In some embodiments, compounds of this embodiment may be included in a pharmaceutical composition or a method of treatment or use described herein.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Plasma concentrations of a subset of exemplar compounds P-37, P-42, and, P-51 in male C57BL/6 mice following IP administration at 10 mg/kg

FIG. 2: Time binned and mean±SD (n=3) HTR counts of a subset of exemplar compounds P-42 and P-51 in male C57BL/6 mice following SC administration over several doses. Psilocin data from: Glatfelter, et al. “Structure-Activity Relationships for Psilocybin, Baeocystin, Aeruginascin, and Related Analogues to Produce Pharmacological Effects in Mice.” ACS Pharmacology & Translational Science 5, no. 11 (November 2022): 1181-96.

FIG. 3: Temperature and locomotor results displayed as mean±SD (n=3) HTR counts of a subset of exemplar compounds P-42 and P-51 in male C57BL/6 mice following SC administration over several doses. Psilocin data from: Glatfelter, et al. “Structure-Activity Relationships for Psilocybin, Baeocystin, Aeruginascin, and Related Analogues to Produce Pharmacological Effects in Mice.” ACS Pharmacology & Translational Science 5, no. 11 (November 2022): 1181-96.

FIG. 4: Average time spent immobile in the ASR-TST model of depression in male ICR mice following administration of a select few exemplar compounds P-42 and P-52.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

The first aspect of this disclosure provides a compound of formula (I) as defined herein.

In embodiments, R⁷, R¹⁰ and R¹¹ are each independently selected from hydrogen, halogen, CN, OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O) R¹³, OS(O)N(R¹³)₂, OSO₂R¹³, OP(O)(OR¹³)₂, OC₁₋₆alkyleneP(O)(OR¹³)₂, S(O) R¹³, S(O) N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
alternatively, R⁶ and R⁷ are combined with the atoms to which they are each attached to form a C₄₋₁₀ heterocycloalkyl or a C₅₋₁₀ heteroaryl, said C₄₋₁₀ heterocycloalkyl and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
alternatively, R⁷ and one of R¹, R², or R³ are combined with the atoms to which they are attached to form a C₅₋₈ heterocycloalkyl,
said C₅₋₈ heterocyclyalkyl being further optionally substituted with one or more substituents selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
R⁸ and R⁹ are combined with the atoms to which they are each attached to form a C₄₋₈ cycloalkyl, C₅₋₈ heterocycloalkyl, C₆₋₁₂ aryl, or C₅₋₁₀ heteroaryl,
said C₄₋₈ cycloalkyl, C₅₋₈ heterocycloalkyl, C₆₋₁₂ aryl, and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
each R¹⁴ is independently selected from hydrogen, C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl,
said C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃.

In some embodiments, R⁸ and R⁹ are combined with the atoms to which they are each attached to form a C₅₋₈ heterocycloalkyl or C₅₋₁₀ heteroaryl, said C₅₋₈ heterocycloalkyl and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃.

In some embodiments, R⁸ and R⁹ are combined to form a C₅₋₈ heterocycloalkyl or C₅₋₁₀ heteroaryl selected from the following:

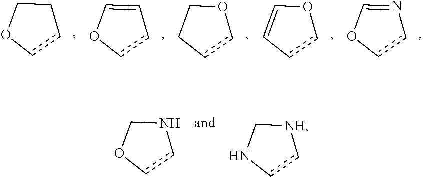

wherein the dashed bond denotes the bond shared with the aromatic ring to which R⁸ and R⁹ are attached,
said C₅₋₈ heterocycloalkyl and C₅₋₁₀ heteroaryl each being further optionally substituted with a substituent selected from halogen, (O), CN, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl and C₁₋₆ haloalkyl.

In some embodiments, R⁸ and R⁹ are combined to form a C₅₋₈ heterocycloalkyl or C₅₋₁₀ heteroaryl selected from the following:

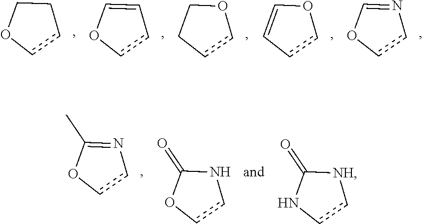

wherein the dashed bond denotes the bond shared with the aromatic ring to which R⁸ and R⁹ are attached.

Typically, the moieties depicted for embodiments when R⁸ and R⁹ together with the atoms to which they are attached form the C₅₋₈ heterocycloalkyl or C₅₋₁₀heteroaryl moieties, the depicted moieties and formula (I) are connected in the orientation as drawn for both formulas.

In some embodiments, R⁸ and R⁹ together with the atoms to which they are attached form a C₅ heterocycloalkyl or C₅ heteroaryl moiety. In some embodiments, the compound of formula (I) may be a compound of formula (II):

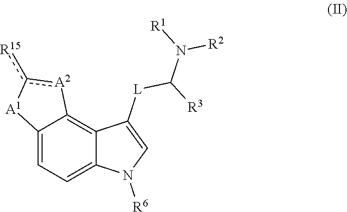

wherein L, R¹, R², R³ and R⁶ are as defined for any aspect or embodiment herein
A¹ and A² are independently selected from O, NR¹⁶, C(H)ₘ;
R¹⁵ is selected from hydrogen, oxo (═O), C₁₋₄alkyl, haloC₁₋₄alkyl;
[symbol] denotes a single or a double covalent bond;
R¹⁶ is selected from hydrogen and C₁₋₆alkyl; and
m is 1 or 2 and is selected according to the valency requirements.

In some embodiments, A¹ is O.

In some embodiments, A² is C(H)ₘ, N or NH.

In some embodiments, A¹ is O, A² is NH and R¹⁵ is oxo.

In some embodiments, A¹ is O, A² is N and R¹⁵ is H.

In some embodiments, A¹ is O, A² is N and R¹⁵ is C₁₋₆alkyl, preferably methyl.

In some embodiments, A¹ is NH, A² is NH and R¹⁵ is oxo.

In some embodiments, R⁹ and R¹⁰ together with the atoms to which they are attached form a C₅ heterocycloalkyl or C₅ heteroaryl moiety. In some embodiments, the compound of formula (I) may be a compound of formula (III):

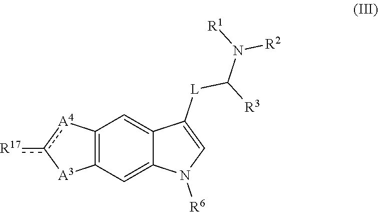

wherein L, R¹, R², R³ and R⁶ are as defined for any aspect or embodiment herein
A¹ and A² are independently selected from O, NR¹⁸, C(H)ₘ;
R¹⁷ is hydrogen, oxo (═O), C₁₋₄alkyl, haloC₁₋₄alkyl;
[symbol] denotes a single or a double covalent bond;
R¹⁸ is selected from hydrogen and C₁₋₆alkyl; and
m is 1 or 2 and is selected according to the valency requirements.

In some embodiments R⁷, R¹⁰ and R¹¹ are each independently selected from hydrogen, halogen, CN, OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)N(R¹³)₂, OC(O)R¹³, OSO₂R¹³, OP(O)(OR¹³)₂, OC₁₋₆alkyleneP(O)(OR¹³)₂, S(O) R¹³, SO₂R¹³, N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NO₂, NHCH₃, SH, SCH₃, SO₂CH₃, and SOCH₃,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, NH and NCH₃;
wherein R¹³ is as defined in any aspect or embodiment herein.

In some embodiments, R⁷, R¹⁰ and R¹¹ are each independently selected from hydrogen, halogen, C₁₋₆ alkyl, C₁₋₆ haloalkyl and OR¹³ wherein R¹³ is selected from hydrogen, C₁₋₆ alkyl and C₁₋₆ haloalkyl.

In some embodiments, R⁷, R¹⁰ and R¹¹ are each hydrogen.

In some embodiments, R⁷, R⁸, R⁹, R¹⁰ and R¹¹ are each independently selected from hydrogen, halogen, CN, OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O) R¹³, OS(O)N(R¹³)₂, OSO₂R¹³, OP(O)(OR¹³)₂, OC₁₋₆alkyleneP(O)(OR¹³)₂, S(O) R¹³, S(O) N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NHS₃;
alternatively, R⁶ and R⁷ are combined with the atoms to which they are each attached to form a C₄₋₁₀ heterocycloalkyl or a C₅₋₁₀ heteroaryl,
said C₄₋₁₀ heterocycloalkyl and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
alternatively, R⁷ and one of R¹, R², or R³ are combined with the atoms to which they are attached to form a C₅₋₈ heterocycloalkyl,
said C₅₋₈ heterocyclyalkyl being further optionally substituted with one or more substituents selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
each R¹⁴ is independently selected from hydrogen, C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl,
said C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
wherein at least two or more of R⁷, R⁸, R⁹, R¹⁰ and R¹¹ are not hydrogen; and
wherein:
when R¹ and R² are each methyl, R³ is hydrogen, R⁶ is selected from hydrogen, methyl, ethyl and propyl, and one of R⁹, R¹⁰ or R¹¹ is fluoro and the other of R⁹, R¹⁰ or R¹¹ are hydrogen, then R⁸ is not selected from OH, OCH₃, OCH₂CH₃, OCH₂CH₂CH₃ and OBn; and
when R¹ and R² are each methyl, R³ is hydrogen, R⁶ is selected from hydrogen, methyl, ethyl and propyl, R⁹ is fluoro, and R¹¹ is hydrogen, then R¹⁰ is not selected from OH, OCH₃, OCH₂CH₃, OCH₂CH₂CH₃ and OBn.

In some embodiments, R⁷, R⁸ R⁹, R¹⁰ and R¹¹ are each independently selected from hydrogen, halogen, CN, OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)N(R¹³)₂, OC(O)R¹³, OSO₂R¹³, OP(O)(OR¹³)₂, OC₁₋₆alkyleneP(O)(OR¹³)₂, S(O) R¹³, SO₂R¹³, N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NO₂, NHCH₃, SH, SCH₃, SO₂CH₃, and SOCH₃,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, NH and NCH₃;
wherein R¹³ is as defined in any one of the foregoing paragraphs;
wherein at least two or more of R⁷, R⁸, R⁹, R¹⁰ and R¹¹ are not hydrogen; and

wherein:

when R¹ and R² are each methyl, R³ is hydrogen, R⁶ is selected from hydrogen, methyl, ethyl and propyl, and when one of R⁹, R¹⁰ and R¹¹ is fluoro and the other of R⁹, R¹⁰ and R¹¹ are hydrogen, then R⁸ is not selected from OH, OCH₃, OCH₂CH₃, OCH₂CH₂CH₃ or OBn; and
when R¹ and R² are each methyl, R³ is hydrogen, R⁶ is selected from hydrogen, methyl, ethyl and propyl, R⁹ is fluoro, and R¹¹ is hydrogen, then R¹⁰ is not selected from OH, OCH₃, OCH₂CH₃, OCH₂CH₂CH₃ and OBn.

In some embodiments, R⁷, R⁸, R⁹, R¹⁰ and R¹¹ are each independently selected from hydrogen, halogen, C₁₋₆ alkyl, C₁₋₆ haloalkyl and OR¹³ wherein R¹³ is selected from hydrogen, C₁₋₆ alkyl and C₁₋₆ haloalkyl,
wherein at least two or more of R⁷, R⁸, R⁹, R¹⁰ and R¹¹ are not hydrogen; and
wherein:
when R¹ and R² are each methyl, R³ is hydrogen, R⁶ is selected from hydrogen, methyl, ethyl and propyl, and one of R⁹, R¹⁰ and R¹¹ are fluoro and the other of R⁹, R¹⁰ and R¹¹ are hydrogen, then R⁸ is not selected from OH, OCH₃, OCH₂CH₃ and OCH₂CH₂CH₃; and
when R¹ and R² are each methyl, R³ is hydrogen, R⁶ is selected from hydrogen, methyl, ethyl and propyl, R⁹ is fluoro, and R¹¹ is hydrogen, then R¹⁰ is not selected from OH, OCH₃, OCH₂CH₃ and OCH₂CH₂CH₃.

In some embodiments, R⁸ is selected from halogen, C₁₋₆ alkyl and OR¹³ wherein R¹³ is selected from hydrogen, C₁₋₆ alkyl and C₁₋₆ haloalkyl.

In some embodiments, R⁹ is selected from halogen, C₁₋₆ alkyl and OR¹³ wherein R¹³ is selected from hydrogen, C₁₋₆ alkyl and C₁₋₆ haloalkyl.

In some embodiments, R⁶ is hydrogen.

In some embodiments, R⁷ is hydrogen.

In some embodiments, R⁷ is selected from hydrogen and methyl, and two of R⁸, R⁹, R¹⁰ and R¹¹ are also H, the others are as defined for any aspect or embodiment herein. In some embodiments, R⁷ is hydrogen, and two of R⁸, R⁹, R¹⁰ and R¹¹ are also H, the others are as defined for any aspect or embodiment herein.

In some embodiments, R⁸ is hydrogen.

In some embodiments, R⁹ is hydrogen.

In some embodiments, R¹⁰ is hydrogen.

In some embodiments, R¹¹ is hydrogen.

In some embodiments, R⁶ and R⁷ are each hydrogen.

In some embodiments, R⁷, R⁸, R⁹, R¹⁰ and R¹¹ are defined by any one of embodiments 1 to 18:

| No. | R⁷ | R⁸ | R⁹ | R¹⁰ | R¹¹ |
|---|---|---|---|---|---|
| 1 | H | C₁₋₆alkyl | OR¹³ | H | H |
| 2 | H | H | OR¹³ | C₁₋₆alkyl | H |
| 3 | C₁₋₆alkyl | OR¹³ | H | H | H |
| 4 | H | OR¹³ | C₁₋₆alkyl | H | H |
| 5 | H | OR¹³ | H | C₁₋₆alkyl | H |
| 6 | H | OR¹³ | H | H | C₁₋₆alkyl |
| 7 | H | OR¹³ | H | Halo | H |
| 8 | H | H | OR¹³ | H | C₁₋₆alkyl |
| 9 | H | OR¹³ | Halo | H | H |
| 10 | H | OR¹³ | H | H | Halo |
| 11 | H | Halo | OR¹³ | H | H |
| 12 | H | H | OR¹³ | H | Halo |
| 13 | H | Halo | Halo | H | H |
| 14 | H | Halo | H | Halo | H |
| 15 | H | Halo | H | H | Halo |
| 16 | H | H | Halo | Halo | H |
| 17 | H | H | Halo | H | Halo |
| 18 | H | H | H | Halo | Halo |

In some embodiments, one of R⁸ and R⁹ is OR¹³.

In some embodiments, any one or more of R⁷, R¹⁰ and R¹¹ is not OR¹³.

In some embodiments, R¹³ is H or C₁₋₆alkyl. In embodiments, R¹³ is H. In embodiments, R¹³ is C₁₋₆alkyl, preferably C₁₋₄alkyl, more preferably methyl.

In some embodiments, one or two of R⁸, R⁹, R¹⁰ and R¹¹ is halo, preferably fluoro. In some embodiments, one of R⁸, R⁹, R¹⁰ and R¹¹ is halo, preferably fluoro. In some embodiments, two of R⁸, R⁹, R¹⁰ and R¹¹ is halo, preferably fluoro, and the others are H.

In some embodiments, any one of R⁷, R⁸, R⁹, R¹⁰ and R¹¹ is C₁₋₆alkyl, preferably C₁₋₄alkyl, more preferably methyl.

In some embodiments, R¹ and R² are each independently selected from C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl and C₄₋₁₄ alkylenecycloalkyl.

In some embodiments, R¹ and R² are each independently selected from C₁₋₄ alkyl.

In some embodiments, at least one of R¹ and R² is not methyl. In some embodiments, both of R¹ and R² are not methyl.

In some embodiments, R¹ and R², together with the nitrogen to which they are attached, form any one of the following:

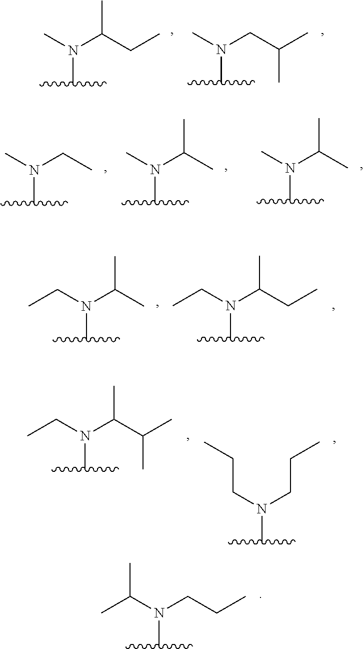

In some embodiments, R¹ and R², together with the nitrogen to which they are attached, form any one of the following:

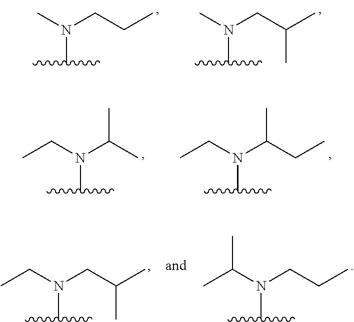

In some embodiments, R¹ and R² are combined with the atoms to which they are attached to form C₃₋₆ heterocycloalkyl, said C₃₋₆ heterocycloalkyl being optionally substituted with one or more substituents independently selected from halogen, CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴ and SO₂R⁴, (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴, wherein R⁴ is as defined in any one of the foregoing paragraphs.

In some embodiments R³ is hydrogen.

In some embodiments, R³ and one of R¹ and R² are combined with the atoms to which they are attached to form a C₃₋₈ heterocycloalkyl, said C₅₋₈ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴, wherein R⁴ is as defined in any one of the foregoing paragraphs.

In some embodiments, L is C₁₋₄ alkylene.

In some embodiments, L is methylene.

In some embodiments, R⁶ is selected from hydrogen and C₁₋₆ alkyl.

In some embodiments, R⁶ is hydrogen.

In some embodiments, the compound of formula (I) is selected from any one of compounds P-1 to P-161 described herein, for example compounds P-1-P-7 and P-37-P-161.

In some embodiments, the compound of formula (I) is selected from any one of compounds P1-P4 and P-125-P-135 or a pharmaceutically acceptable salt, solvate, tautomer, N-oxide, stereoisomer, metabolite, polymorph or prodrug thereof.

In some embodiments, the compound of formula (I) is selected from any one of compounds P-5-P-7, P-37-P-124 and P-136-P161, or a pharmaceutically acceptable salt, solvate, tautomer, N-oxide, stereoisomer, metabolite, polymorph or prodrug thereof.

In some embodiments, the compound is selected from any one of P1-P3, P7-P9, P11-P14 and P-37-P49, P51-P-152, P-153-P-155, P-157-P-158 and P-160-P-161.

In another aspect of the present disclosure there is provided a medicament comprising a compound of formula (I) according to any one of the herein disclosed embodiments, or a pharmaceutically acceptable salt, solvate, tautomer, N-oxide, stereoisomer, metabolite, polymorph or prodrug thereof.

In another aspect of the present disclosure there is provided a pharmaceutical composition comprising a compound of formula (I) according to any one of the herein disclosed embodiments, or a pharmaceutically acceptable salt, solvate, tautomer, N-oxide, stereoisomer, metabolite, polymorph or prodrug thereof, and a pharmaceutically acceptable excipient.

In another aspect the present disclosure provides a pharmaceutical composition comprising a compound according to any one of the herein disclosed embodiments, or a pharmaceutically acceptable salt, solvate, tautomer, N-oxide, stereoisomer, metabolite, polymorph or prodrug thereof, an additional therapeutic agent, and a pharmaceutically acceptable excipient.

In another aspect of the present disclosure there is provided a method of treating a disease, disorder or condition by activation of a serotonin receptor, the method comprising administering to a subject in need thereof a compound of formula (I):

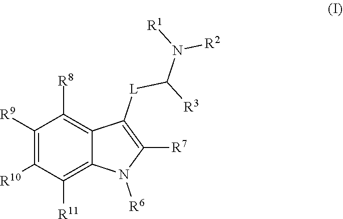

or a pharmaceutically acceptable salt, solvate, tautomer, N-oxide, stereoisomer, metabolite, polymorph or prodrug thereof,
wherein
R¹ and R² are each independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴ and SO₂R⁴,
said C₃₋₈ cycloalkyl, C₄₋₁₄ alkylenecycloalkyl, C₃-C₈ heterocycloalkyl, C₆₋₁₂ aryl, C₇₋₁₈ alkylenearyl, C₅₋₁₀ heteroaryl, and C₆₋₁₆ alkyleneheteroaryl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
alternatively R¹ and R² are combined with the atoms to which they are attached to form a C₃₋₈ heterocycloalkyl including 1 or 2 additional ring heteromoieties selected from O, S, S(O), SO₂, N and NR⁴,
said C₃₋₈ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆alkenyl, C₂₋₆ haloalkenyl, C₂₋₆ alkynyl, C₂₋₆ haloalkynyl, C₁₋₈ alkylamino, C₁₋₈ alkylsulfonyl, C₃₋₆ cycloalkyl and C₃₋₆ heterocycloalkyl including 1 or 2 ring heteromoieties selected from O, S, N, S(O), SO₂ and NR⁴;
R³ is selected from hydrogen, C₁₋₆ alkyl, C₃₋₈ cycloalkyl, or C₄₋₁₄ alkylenecycloalkyl;
alternatively R³ and one of R¹ and R² are combined with the atoms to which they are attached to form a C₃₋₁₂ heterocycloalkyl,
said C₃₋₁₂ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
each R⁴ is independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, and C₃₋₇ heterocycloalkyl including 1 or 2 ring heteromoieties selected from O, S, S(O), SO₂, N and NR⁵,
said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl and C₃₋₇ heterocycloalkyl each being optionally substituted with one or more substituents independently selected from halogen, CN, C₁₋₈ alkoxy, C₁₋₈ alkylamino, C₁₋₈ alkylsulfonyl, CO₂R⁵, C(O)N(R⁵)₂, OR⁵, N(R⁵)₂, NO₂, SR⁵ and SO₂R⁵,
said C₃-C₇ cycloalkyl and C₃₋₇ heterocycloalkyl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N and NR⁵;
each R⁵ is independently selected from hydrogen, C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl,
said C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
L is selected from C₁₋₄ alkylene, C₂-C₄ alkenylene and C₂-C₄ alkynylene;
R⁶ is selected from hydrogen, C₁₋₆ alkyl, C₂₋₆ alkenyl, C₂₋₆ alkynyl, C₁₋₆ alkyleneP(O)(OR¹²)₂, C(O)R¹², CO₂R¹², C(O)N(R¹²)₂, S(O) R¹² and SO₂R¹², C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂R¹², C(O)N(R¹²)₂, OR¹², N(R¹²)₂, NO₂, SR¹² and SO₂R¹²;
said C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹²;
each R¹² is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
R⁷, R⁸, R⁹, R¹⁰ and R¹¹ are each independently selected from hydrogen, halogen, CN, OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O) R¹³, OS(O)N(R¹³)₂, OSO₂R¹³, OP(O)(OR¹³)₂, OC₁₋₆alkyleneP(O)(OR¹³)₂, S(O) R¹³, S(O) N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
alternatively, R⁶ and R⁷ are combined with the atoms to which they are each attached to form a C₄₋₁₀ heterocycloalkyl or a C₅₋₁₀ heteroaryl,
said C₄₋₁₀ heterocycloalkyl and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
alternatively, R⁷ and one of R¹, R², or R³ are combined with the atoms to which they are attached to form a C₅₋₈ heterocycloalkyl,
said C₅₋₈ heterocyclyalkyl being further optionally substituted with one or more substituents selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
alternatively, R⁸ and R⁹, or R⁹ and R¹⁰, or R¹⁰ and R¹¹ are combined with the atoms to which they are each attached to form a C₄₋₈ cycloalkyl, C₅₋₈ heterocycloalkyl, C₆₋₁₂ aryl, or C₅₋₁₀ heteroaryl,
said C₄₋₈ cycloalkyl, C₅₋₈ heterocycloalkyl, C₆₋₁₂ aryl, and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
each R¹⁴ is independently selected from hydrogen, C₁₋₆ alkyl, C₂-C₆ alkenyl, C₂-C₆ alkynyl, C₁-C₆ haloalkyl, C₃-C₇ cycloalkyl, C₃₋₁₀ heterocycloalkyl, C₆₋₁₂ aryl and C₅₋₁₀ heteroaryl,
said C₁₋₆ alkyl, C₂-C₆ alkenyl, C₂-C₆ alkynyl, C₁-C₆ haloalkyl, C₃-C₇ cycloalkyl, C₃₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
wherein at least two or more of R⁷, R⁸, R⁹, R¹⁰ and R¹¹ are not hydrogen;
wherein:
when R¹ and R² are each methyl, R³ is hydrogen, R⁶ is selected from hydrogen, methyl, ethyl and propyl, and one of R⁹, R¹⁰ and R¹¹ is fluoro and the other of R⁹, R¹⁰ and R¹¹ are hydrogen, then R⁸ is not selected from OH, OCH₃, OCH₂CH₃, OCH₂CH₂CH₃ or OBn; and
when R¹ and R² are each methyl, R³ is hydrogen, R⁶ is selected from hydrogen, methyl, ethyl and propyl, R⁹ is fluoro, and R¹¹ is hydrogen, then R¹⁰ is not selected from OH, OCH₃, OCH₂CH₃, OCH₂CH₂CH₃ and OBn; and
wherein the compound is not selected from the following:

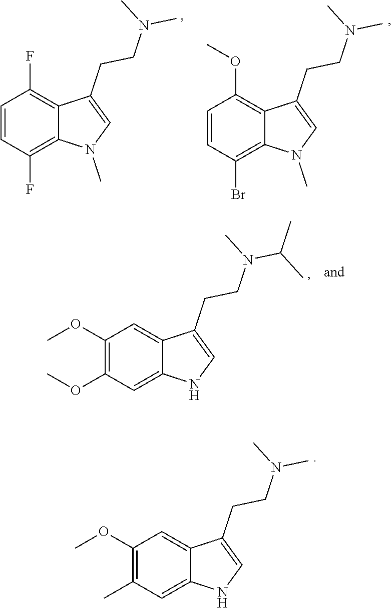

In some embodiments of the method, the compound is not selected from the following:

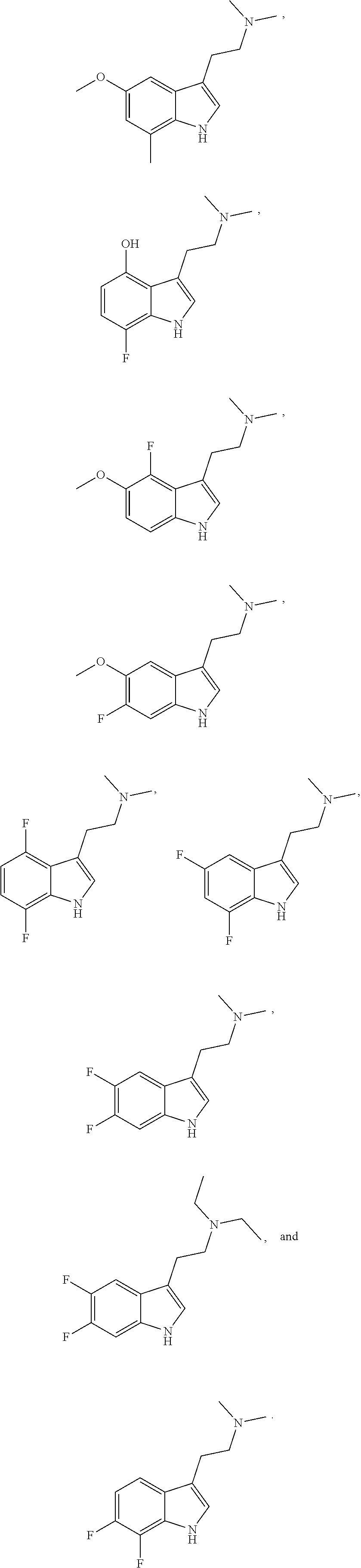

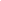

In some embodiments of the method, R⁷, R¹⁰ and R¹¹ are each independently selected from hydrogen, halogen, CN, OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O) R¹³, OS(O)N(R¹³)₂, OSO₂R¹³, OP(O)(OR¹³)₂, OC₁₋₆alkyleneP(O)(OR¹³)₂, S(O) R¹³, S(O) N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
alternatively, R⁶ and R⁷ are combined with the atoms to which they are each attached to form a C₄₋₁₀ heterocycloalkyl or a C₅₋₁₀ heteroaryl,
said C₄₋₁₀ heterocycloalkyl and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
alternatively, R⁷ and one of R¹, R², or R³ are combined with the atoms to which they are attached to form a C₅₋₈ heterocycloalkyl,
said C₅₋₈ heterocyclyalkyl being further optionally substituted with one or more substituents selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
R⁸ and R⁹ are combined with the atoms to which they are each attached to form a C₄₋₈ cycloalkyl, C₅₋₈ heterocycloalkyl, C₆₋₁₂ aryl, or C₅₋₁₀ heteroaryl,
said C₄₋₈ cycloalkyl, C₅₋₈ heterocycloalkyl, C₆₋₁₂ aryl, and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
each R¹⁴ is independently selected from hydrogen, C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl,
said C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃.

In some embodiments of the method, R⁸ and R⁹ are combined with the atoms to which they are each attached to form a C₅₋₈ heterocycloalkyl or C₅₋₁₀ heteroaryl, said C₅₋₈ heterocycloalkyl and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃.

In some embodiments of the method, R⁸ and R⁹ are combined to form a C₅₋₈ heterocycloalkyl or C₅₋₁₀ heteroaryl selected from the following:

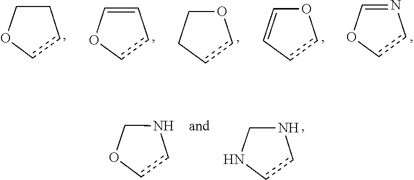

wherein the dashed bond denotes the bond shared with the aromatic ring to which R⁸ and R⁹ are attached,
said C₅₋₈ heterocycloalkyl and C₅₋₁₀ heteroaryl each being further optionally substituted with a substituent selected from halogen, (O), CN, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl and C₁₋₆ haloalkyl.

In some embodiments of the method, R⁸ and R⁹ are combined to form a C₅₋₈ heterocycloalkyl or C₅₋₁₀ heteroaryl selected from the following:

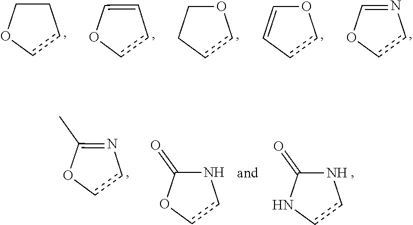

wherein the dashed bond denotes the bond shared with the aromatic ring to which R⁸ and R⁹ are attached.

In some embodiments of the method, R⁷, R¹⁰ and R¹¹ are each independently selected from hydrogen, halogen, CN, OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)N(R¹³)₂, OC(O)R¹³, OSO₂R¹³, OP(O)(OR¹³)₂, OC₁₋₆alkyleneP(O)(OR¹³)₂, S(O) R¹³, SO₂R¹³, N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NO₂, NHCH₃, SH, SCH₃, SO₂CH₃, and SOCH₃,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, NH and NCH₃;
wherein R¹³ is as defined in any one of the foregoing paragraphs.

In some embodiments of the method, R⁷, R¹⁰ and R¹¹ are each independently selected from hydrogen, halogen, C₁₋₆ alkyl, C₁₋₆ haloalkyl and OR¹³ wherein R¹³ is selected from hydrogen, C₁₋₆ alkyl and C₁₋₆ haloalkyl.

In some embodiments of the method, R⁷, R¹⁰ and R¹¹ are each hydrogen.

In some embodiments of the method, R⁷, R⁸, R⁹, R¹⁰ and R¹¹ are each independently selected from hydrogen, halogen, CN, OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O) R¹³, OS(O)N(R¹³)₂, OSO₂R¹³, OP(O)(OR¹³)₂, OC₁₋₆alkyleneP(O)(OR¹³)₂, S(O) R¹³, S(O) N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NHS₃;
alternatively, R⁶ and R⁷ are combined with the atoms to which they are each attached to form a C₄₋₁₀ heterocycloalkyl or a C₅₋₁₀ heteroaryl,
said C₄₋₁₀ heterocycloalkyl and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
alternatively, R⁷ and one of R¹, R², or R³ are combined with the atoms to which they are attached to form a C₅₋₈ heterocycloalkyl,
said C₅₋₈ heterocyclyalkyl being further optionally substituted with one or more substituents selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
each R¹⁴ is independently selected from hydrogen, C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl,
said C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
wherein at least two or more of R⁷, R⁸, R⁹, R¹⁰ and R¹¹ are not hydrogen; and
wherein:
when R¹ and R² are each methyl, R³ is hydrogen, R⁶ is selected from hydrogen, methyl, ethyl and propyl, and one of R⁹, R¹⁰ and R¹¹ is fluoro and the other of R⁹, R¹⁰ and R¹¹ are hydrogen, then R⁸ is not selected from OH, OCH₃, OCH₂CH₃, OCH₂CH₂CH₃ or OBn; and
when R¹ and R² are each methyl, R³ is hydrogen, R⁶ is selected from hydrogen, methyl, ethyl and propyl, R⁹ is fluoro, and R¹¹ is hydrogen, then R¹⁰ is not selected from OH, OCH₃, OCH₂CH₃, OCH₂CH₂CH₃ and OBn.

In some embodiments of the method, R⁷, R⁸ R⁹, R¹⁰ and R¹¹ are each independently selected from hydrogen, halogen, CN, OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)N(R¹³)₂, OC(O)R¹³, OSO₂R¹³, OP(O)(OR¹³)₂, OC₁₋₆alkyleneP(O)(OR¹³)₂, S(O) R¹³, SO₂R¹³, N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NO₂, NHCH₃, SH, SCH₃, SO₂CH₃, and SOCH₃,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, NH and NCH₃;
wherein R¹³ is as defined in any one of the foregoing paragraphs;
wherein at least two or more of R⁷, R⁸, R⁹, R¹⁰ and R¹¹ are not hydrogen; and
wherein:
when R¹ and R² are each methyl, R³ is hydrogen, R⁶ is selected from hydrogen, methyl, ethyl and propyl, and one of R⁹, R¹⁰ and R¹¹ is fluoro and the other of R⁹, R¹⁰ and R¹¹ are hydrogen, then R⁸ is not selected from OH, OCH₃, OCH₂CH₃, OCH₂CH₂CH₃ or OBn;
when R¹ and R² are each methyl, R³ is hydrogen, R⁶ is selected from hydrogen, methyl, ethyl and propyl, R⁹ is fluoro, and R¹¹ is hydrogen, then R¹⁰ is not selected from OH, OCH₃, OCH₂CH₃, OCH₂CH₂CH₃ and OBn.

In some embodiments of the method, R⁷, R⁸, R⁹, R¹⁰ and R¹¹ are each independently selected from hydrogen, halogen, C₁₋₆ alkyl, C₁₋₆ haloalkyl and OR¹³ wherein R¹³ is selected from hydrogen, C₁₋₆ alkyl and C₁₋₆ haloalkyl;
wherein at least two or more of R⁷, R⁸, R⁹, R¹⁰ and R¹¹ are not hydrogen; and
wherein:
when R¹ and R² are each methyl, R³ is hydrogen, R⁶ is selected from hydrogen, methyl, ethyl and propyl, and one of R⁹, R¹⁰ and R¹¹ is fluoro and the other of R⁹, R¹⁰ and R¹¹ are hydrogen, then R⁸ is not selected from OH, OCH₃, OCH₂CH₃ and OCH₂CH₂CH₃; and
when R¹ and R² are each methyl, R³ is hydrogen, R⁶ is selected from hydrogen, methyl, ethyl and propyl, R⁹ is fluoro, and R¹¹ is hydrogen, then R¹⁰ is not selected from OH, OCH₃, OCH₂CH₃ and OCH₂CH₂CH₃.

In some embodiments of the method, R⁸ is selected from halogen, C₁₋₆ alkyl and OR¹³ wherein R¹³ is selected from hydrogen, C₁₋₆ alkyl and C₁₋₆ haloalkyl.

In some embodiments of the method, R⁹ is selected from halogen, C₁₋₆ alkyl and OR¹³ wherein R¹³ is selected from hydrogen, C₁₋₆ alkyl and C₁₋₆ haloalkyl.

In some embodiments of the method, R¹ and R² are each independently selected from C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl and C₄₋₁₄ alkylenecycloalkyl.

In some embodiments of the method, R¹ and R² are each independently selected from C₁₋₄ alkyl.

In some embodiments of the method, R¹ and R², together with the nitrogen to which they are attached, form any one of the following:

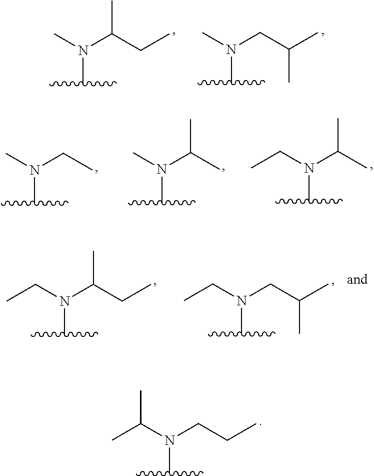

In some embodiments of the method, R¹ and R², together with the nitrogen to which they are attached, form any one of the following:

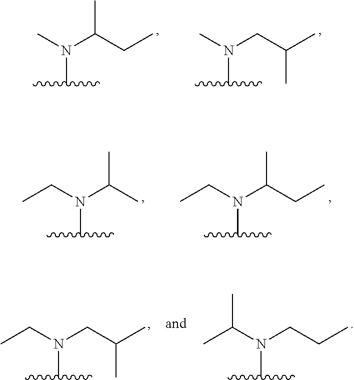

In some embodiments of the method, R¹ and R² are combined with the atoms to which they are attached to form C₃₋₆ heterocycloalkyl, said C₃₋₆ heterocycloalkyl being optionally substituted with one or more substituents independently selected from halogen, CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴ and SO₂R⁴, (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴, wherein R⁴ is as defined in any one of the foregoing paragraphs.

In some embodiments of the method, R³ is hydrogen.

In some embodiments of the method, R³ and one of R¹ and R² are combined with the atoms to which they are attached to form a C₃₋₈ heterocycloalkyl, said C₅₋₈ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴, wherein R⁴ is as defined in any one of the foregoing paragraphs.

In some embodiments of the method, L is C₁₋₄ alkylene.

In some embodiments of the method, L is methylene.

In some embodiments of the method, R⁶ is selected from hydrogen and C₁₋₆ alkyl.

In some embodiments of the method, R⁶ is hydrogen.

In some embodiments of the method, the compound of formula (I) as defined in any aspect or embodiment herein.

In some embodiments, the compound of formula (I) is selected from any one of claims 1-3, 6-14 and 37-161. In some embodiments, the compound is selected from any one of 1-3, 7-9, 11-14 and 37-49, 51-152, 153-155, 157-158 and 160-161.

In another aspect of the present disclosure there is provided a method of treating a disease, disorder or condition by activation of a serotonin receptor, the method comprising administering to a subject in need thereof a compound of formula (I) according to any one of the herein disclosed embodiments, or a pharmaceutically acceptable salt, solvate, tautomer, N-oxide, stereoisomer, metabolite, polymorph or prodrug thereof, in combination with another known agent useful for treatment of a disease, disorder or condition by activation of a serotonin receptor.

In another aspect of the present disclosure there is provided a method of treating a mental illness, the method comprising administering to a subject in need thereof a compound of formula (I) according to any one of the herein disclosed embodiments, or a pharmaceutically acceptable salt, solvate, tautomer, N-oxide, stereoisomer, metabolite, polymorph or prodrug thereof.

In embodiments, the mental illness is selected from anxiety disorders; depression; mood disorders; psychotic disorders; impulse control and addiction disorders; drug addiction; obsessive-compulsive disorder (OCD); post-traumatic stress disorder (PTSD); stress response syndromes; dissociative disorders; depersonalization disorder; factitious disorders; sexual and gender disorders; somatic symptom disorders; hallucinations; delusions; psychosis; and combinations thereof.

In another aspect of the present disclosure there is provided a method for treating a central nervous system (CNS) disease, disorder or condition and/or a neurological disease, disorder or condition, the method comprising administering to a subject in need thereof a compound of formula (I) according to any one of the herein disclosed embodiments, or a pharmaceutically acceptable salt, solvate, tautomer, N-oxide, stereoisomer, metabolite, polymorph or prodrug thereof.

In embodiments, the CNS disease, disorder or condition and/or neurological disease, disorder or condition is selected from neurological diseases including neurodevelopmental diseases and neurodegenerative diseases such as Alzheimer's disease; presenile dementia; senile dementia; vascular dementia; Lewy body dementia; cognitive impairment, Parkinson's disease and Parkinsonian related disorders such as Parkinson dementia, corticobasal degeneration, and supranuclear palsy; epilepsy; CNS trauma; CNS infections; CNS inflammation; stroke; multiple sclerosis; Huntington's disease; mitochondrial disorders; Fragile X syndrome; Angelman syndrome; hereditary ataxias; neuro-otological and eye movement disorders; neurodegenerative diseases of the retina amyotrophic lateral sclerosis; tardive dyskinesias; hyperkinetic disorders; attention deficit hyperactivity disorder and attention deficit disorders; restless leg syndrome; Tourette's syndrome; schizophrenia; autism spectrum disorders; tuberous sclerosis; Rett syndrome; cerebral palsy; disorders of the reward system including eating disorders such as anorexia nervosa and bulimia nervosa; binge eating disorder, trichotillomania, dermotillomania, nail biting; migraine; fibromyalgia; and peripheral neuropathy of any etiology, and combinations thereof.

In another aspect of the present disclosure there is provided a method for increasing neuronal plasticity and/or increasing dendritic spine density, the method comprising contacting a neuronal cell with a compound of formula (I) according to any one of the herein disclosed embodiments, or a pharmaceutically acceptable salt, solvate, tautomer, N-oxide, stereoisomer, metabolite, polymorph or prodrug thereof, in an amount sufficient to increase neuronal plasticity and/or increase dendritic spine density of the neuronal cell.

In another aspect the present disclosure provides methods of treating weight, comprising administering an effective amount of a compound of the invention to a subject in need thereof. Treatment of weight may include treating weight gain; weight loss; metabolic disorder; weight gain associated with pharmaceutical intervention; weight gain associated with a mental illness (including those described herein); eating disorders such as anorexia, bulimia, cachexia, etc.; eating behaviour; obesity; diabetes; insulin resistance; pre-diabetes; glucose intolerance; hyperlipidemia; and cardiovascular disease.

In another aspect the present disclosure provides a method for activating a serotonin receptor in a cell, either in a biological sample or in a patient, comprising administering a compound of formula (I) as defined in any one of the herein disclosed embodiments to the cell.

Any embodiment herein shall be taken to apply mutatis mutandis to any other embodiment unless specifically stated otherwise.

The present disclosure is not to be limited in scope by the specific embodiments described herein, which are intended for the purpose of exemplification only. Functionally-equivalent products, compositions and methods are clearly within the scope of the invention, as described herein.

### Definitions

For purposes of interpreting this specification, terms used in the singular will also include the plural and vice versa.

As used herein, except where the context requires otherwise, the term “comprise” and variations of the term, such as “comprising”, “comprises” and “comprised”, are not intended to exclude further additives, components, integers or steps.

The terms “treatment” or “treating” of a subject includes delaying, slowing, stabilizing, curing, healing, alleviating, relieving, altering, remedying, less worsening, ameliorating, improving, or affecting the disease or condition, the sign or symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of signs or symptoms or making the injury, pathology or condition more tolerable to the individual; slowing in the rate of degeneration or decline; making the final point of degeneration less debilitating.

In particularly preferred embodiments, the methods of the present invention can be to prevent or reduce the severity, or inhibit or minimise progression, of a sign or symptom of a disease or condition as described herein. As such, the methods of the present invention have utility as treatments as well as prophylaxes.

As used herein, “preventing” or “prevention” is intended to refer to at least the reduction of likelihood of the risk of (or susceptibility to) acquiring a disease or disorder (i.e., causing at least one of the clinical signs or symptoms of the disease not to develop in an individual that may be exposed to or predisposed to the disease but does not yet experience or display signs or symptoms of the disease). Biological and physiological parameters for identifying such patients are provided herein and are also well known by physicians.

Herein, the term “subject” or “patient” can be used interchangeably with each other. The term “individual” or “patient” refers to an animal that is treatable by the compound and/or method, respectively, including but not limited to, for example, dogs, cats, horses, sheep, pigs, cows, and the like, as well as human, non-human primates. Unless otherwise specified, the “subject” or “patient” may include both male and female genders. Further, it also includes a subject or patient, preferably a human, suitable for receiving treatment with a pharmaceutical composition and/or method of the present invention.

The term “selective” means a greater activity against a first target (e.g., a 5-HT receptor subtype) relative to a second target (e.g., a second 5-HT receptor subtype). In some embodiments a compound has a selectivity of at least 1.25-fold, at least 1.5 fold, at least 2-fold, at least 3-fold, at least 4-fold, at least 5-fold, at least 6-fold, at least 10-fold or at least 100-fold greater towards a first target relative to a second target. In some embodiments, a compound described herein is selective towards the 5-HT_{2A} receptor relative to one or more other 5-HT receptor subtypes such as 5-HT_{2B} and/or 5-HT_{2C}, preferably 5-HT_{2B}. In some embodiments, a compound described herein is selective towards the 5-HT_{2C} receptor relative to one or more other 5-HT receptor subtypes such as 5-HT_{2A} and/or 5-HT_{2B}, preferably 5-HT_{2B}.

“About” as used herein when referring to a measurable value such as an amount, a temporal duration, and the like, is meant to encompass variations of ±20% or ±10%, in some instances ±5%, in some instances ±1%, and in some instances ±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As used herein the term “alkyl” refers to a straight or branched chain hydrocarbon radical having from one to twelve carbon atoms, or any range between, i.e. it contains 1, 2, 3, 4, 5, 6, 7, 8, 9, 10, 11 or 12 carbon atoms. The alkyl group is optionally substituted with substituents. Examples of “alkyl” as used herein include, but are not limited to, methyl, ethyl, n-propyl, isopropyl, n-butyl, isobutyl, t-butyl, n-pentyl, isopentyl, and the like.

As used herein, the terms “C₁-C₂ alkyl”, “C₁-C₃ alkyl” and “C₁-C₆ alkyl” refer to an alkyl group, as defined herein, containing at least 1, and at most 2, 3 or 6 carbon atoms respectively, or any range in between (eg alkyl groups containing 2-5 carbon atoms are also within the range of C₁-C₆).

The term “alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene.

The term “alkenyl” whether it is used alone or as part of another group, means a straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₂₋₆ alkylene means an alkylene group having 2, 3, 4, 5 or 6 carbon atoms. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl.

The term “alkynyl” as used herein, whether it is used alone or as part of another group, means straight or branched chain, unsaturated alkynyl groups containing at least one triple bond. The number of carbon atoms that are possible in the referenced alkyl group are indicated by the prefix “Cₙ₁₋ₙ₂”. For example, the term C₂₋₆ alkynyl means an alkynyl group having 2, 3, 4, 5 or 6 carbon atoms. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl.

The term “cycloalkyl” is intended to include mono-, bi- or tricyclic alkyl groups. The number of carbon atoms that are possible in the referenced cycloalkyl group are indicated by the prefix “Cₙ₁₋ₙ₂”. For example, the term C₃₋₈ cycloalkyl means an cycloalkyl group having 3, 4, 5, 6, 7 or 8 carbon atoms. In some embodiments, cycloalkyl groups have from 3 to 12, from 3 to 10, from 3 to 8, from 3 to 6, from 3 to 5 carbon atoms in the ring(s). In some embodiments, cycloalkyl groups have 5 or 6 ring carbon atoms. Examples of monocyclic cycloalkyl groups include, but are not limited to, cyclopropyl, cyclobutyl, cyclopentyl, cyclohexyl, cycloheptyl, and cyclooctyl. In some embodiments, the cycloalkyl group has from 3 to 8, from 3 to 7, from 3 to 6, from 4 to 6, from 3 to 5, or from 4 to 5 ring carbon atoms. Bi- and tricyclic ring systems include bridged, spiro, and fused cycloalkyl ring systems. Examples of bi- and tricyclic ring cycloalkyl systems include, but are not limited to, bicyclo[2.1.1]hexanyl, bicyclo[2.2.1]heptanyl, adamantyl, and decalinyl.

The term “alkylenecycloalkyl” refers to a radical having an alkyl component and a cycloalkyl component, where the alkyl component links the cycloalkyl component to the point of attachment. The alkyl component is as defined above, except that the alkyl component is at least divalent, an alkylene, to link to the cycloalkyl component and to the point of attachment. In some instances, the alkyl component can be absent. The alkyl component can include any number of carbons, such as C₁₋₆, C₁₋₂, C₁₋₃, C₁₋₄, C₁₋₅, C₂₋₃, C₂₋₄, C₂₋₅, C₂₋₆, C₃₋₄, C₃₋₅, C₃₋₆, C₄₋₅, C₄₋₆ and C₅₋₆. The cycloalkyl component is as defined herein. The numerical range from x to y in “C_{x-y} alkylenecycloalkyl” relates to the total number of alkyl carbons and cycloalkyl ring atoms. Exemplary alkylenecycloalkyl groups include, but are not limited to, methylenecyclopropyl, methylenecyclobutyl, methylenecyclopentyl and methylenecyclohexyl.

The term “aryl” refers to an aromatic ring system having any suitable number of ring atoms and any suitable number of rings. The number of carbon atoms that are possible in the referenced aryl group are indicated by the prefix “Cₙ₁₋ₙ₂”. For example, the term C₆₋₁₂ aryl means an aryl group having 6, 7, 8, 9, 10, 11 or 12 carbon atoms. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl.

The term “alkylenearyl” refers to a radical having an alkyl component and an aryl component, where the alkyl component links the aryl component to the point of attachment. The alkyl component is as defined above, except that the alkyl component is at least divalent, an alkylene, to link to the aryl component and to the point of attachment. The alkyl component can include any number of carbons, such as C₁₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The aryl component is as defined above. The numerical range from x to y in “C_{x-y} alkylenearyl” relates to the total number of alkyl carbons and aryl ring atoms. Examples of alkylenearyl groups include, but are not limited to, benzyl and ethylenephenyl.

As used herein, the term “alkoxy” refers to an alkyl group as defined herein covalently bound via an O linkage. The alkoxy group is optionally substituted with substituents. Examples of “alkoxy” as used herein include, but are not limited to methoxy, ethoxy, propoxy, isoproxy, butoxy, iso-butoxy, tert-butoxy and pentoxy.

As used herein, the terms “C₁-C₂ alkoxy”, “C₁-C₃ alkoxy” and “C₁-C₆ alkoxy” refer to an alkoxy group, as defined herein, containing at least 1, and at most 2, 3 or 6 carbon atoms respectively, or any range in between (eg alkoxy groups containing 2-5 carbon atoms are also within the range of C₁-C₆).

As used herein, the term “alkylamine” refers to an alkyl group as defined herein having one or more amino groups. The amino groups can be primary, secondary or tertiary. The alkyl amine can be further substituted with a hydroxy group to form an amino-hydroxy group. Examples of alkylamines include, but are not limited to, ethyl amine, propyl amine, isopropyl amine, ethylene diamine and ethanolamine. The amino group can link the alkyl amine to the point of attachment with the rest of the compound, be at the omega position of the alkyl group, or link together at least two carbon atoms of the alkyl group.

As used herein, the terms “C₁-C₂ alkylamine”, “C₁-C₃ alkylamine” and “C₁-C₆ alkylamine” refer to an alkylamine group, as defined herein, containing at least 1, and at most 2, 3 or 6 carbon atoms respectively, or any range in between (e.g., alkylamine groups containing 2-5 carbon atoms are also within the range of C₁-C₆).

As used herein, the term “alkylsulfonyl” refers to an alkyl group as defined herein having one or more sulfonyl groups. The sulfonyl group can link the alkylsulfonyl to the point of attachment with the rest of the compound, be at the omega position of the alkyl group, or link together at least two carbon atoms of the alkyl group.

As used herein, the terms “C₁-C₂ alkylsulfonyl”, “C₁-C₃ alkylsulfonyl” and “C₁-C₆ alkylsulfonyl” refer to an alkylsulfonyl group, as defined herein, containing at least 1, and at most 2, 3 or 6 carbon atoms respectively, or any range in between (e.g., alkylsulfonyl groups containing 2-5 carbon atoms are also within the range of C₁-C₆).

The term “heteroatom” as used herein means an atom of any element other than carbon or hydrogen. Examples of heteroatoms include nitrogen, oxygen, sulfur and phosphorus. Preferred heteroatoms include N, O and S, preferably N and O.

The term “heteromoiety” as used herein means a chemical group comprising a heteroatom. Examples of heteromoieties include O, S, S(O), SO₂, N and NH.

A “substituent” as used herein, refers to a molecular moiety that is covalently bonded to an atom within a molecule of interest. For example, a “ring substituent” may be a moiety such as a halogen, alkyl group, or other substituent described herein that is covalently bonded to an atom, preferably a carbon or nitrogen atom, that is a ring member. The term “substituted,” as used herein, means that any one or more hydrogens on the designated atom is replaced with a selection from the indicated substituents, provided that the designated atom's normal valence is not exceeded, and that the substitution results in a stable compound, ie, a compound that can be isolated, characterized and tested for biological activity.

The terms “optionally substituted” or “may be substituted” and the like, as used throughout the specification, denotes that the group may or may not be further substituted or fused (so as to form a polycyclic system), with one or more non-hydrogen substituent groups. Suitable chemically viable substituents for a particular functional group will be apparent to those skilled in the art.

Examples of substituents include but are not limited to C₁-C₆ alkyl, C₁-C₆ haloalkyl, C₁-C₆ haloalkoxy, C₁-C₆ hydroxyalkyl, C₃-C₇ heterocyclyl, C₃-C₇ cycloalkyl, C₁-C₆ alkoxy, C₁-C₆ alkylsulfanyl, C₁-C₆ alkylsulfenyl, C₁-C₆ alkylsulfonyl, C₁-C₆ alkylsulfonylamino, arylsulfonoamino, alkylcarboxy, alkylcarboxyamide, oxo, hydroxy, mercapto, amino, acyl, carboxy, carbamoyl, aryl, aryloxy, heteroaryl, aminosulfonyl, aroyl, aroylamino, heteroaroyl, acyloxy, aroyloxy, heteroaroyloxy, alkoxycarbonyl, nitro, cyano, halo, ureido, C₁-C₆ perfluoroalkyl. Preferably the substituents include amino, halo, C₁-C₆ alkyl, amido, hydroxyl.

As used herein, the term “halogen” refers to fluorine (F), chlorine (Cl), bromine (Br), or iodine (I) and the term “halo” refers to the halogen radicals fluoro (—F), chloro (—Cl), bromo (—Br), and iodo (—I). Preferably, ‘halo’ is fluoro or chloro.

As used herein, the term “haloalkyl” refers to an alkyl group as defined herein in which one or more (up to all) of the available hydrogen atoms have been replaced with a halogen. In some instances, the term “perfluoro” can be used to define a compound or radical where all the hydrogens are replaced with fluorine. For example, perfluoromethyl refers to 1,1,1-trifluoromethyl.

As used herein, the terms “C₁-C₂ haloalkyl”, “C₁-C₃ haloalkyl” and “C₁-C₆ haloalkyl” refer to a haloalkyl group, as defined herein, containing at least 1, and at most 2, 3 or 6 carbon atoms respectively, or any range in between (e.g. haloalkyl groups containing 2-5 carbon atoms are also within the range of C₁-C₆).

For example a C₁ haloalkyl group could be, but is not limited to, fluoromethyl, or difluoromethyl, or trifluoromethyl.

As used herein, the term “haloalkenyl” refers to an alkenyl group as defined above in which one or more of the available hydrogen atoms have been replaced with a halogen. Thus, for example, “C₁₋₆ haloalkenyl” (or “C₁-C₆ haloalkenyl”) refers to a C₁ to C₆ linear or branched alkenyl group as defined above with one or more halogen substituents.

As used herein, the term “haloalkynyl” refers to an alkynyl group as defined above in which one or more of the available hydrogen atoms have been replaced with a halogen. Thus, for example, “C₁₋₆ haloalkynyl” (or “C₁-C₆ haloalkynyl”) refers to a C₁ to C₆ linear or branched alkynyl group as defined above with one or more halogen substituents.

As used herein the term haloalkoxy refers to an alkoxy group as defined herein substituted with at least one halogen.

The term “amino” or “amine” refers to the group —NH₂.

The term “substituted amino” or “secondary amino” refers to an amino group having a hydrogen replaced with, for example a C₁-C₆ alkyl group (“C₁-C₆ alkylamino”), an aryl or aralkyl group (“arylamino”, “aralkylamino”) and so on. C₁-C₃ alkylamino groups are preferred, such as for example, methylamino (NHMe), ethylamino (NHEt) and propylamino (NHPr).

The term “disubstituted amino” or “tertiary amino” refers to an amino group having the two hydrogens replaced with, for example a C₁-C₆alkyl group, which may be the same or different (“dialkylamino”), an aryl and alkyl group (“aryl(alkyl)amino”) and so on. Di(C₁-C₃alkyl)amino groups are preferred, such as for example, dimethylamino (NMe₂), diethylamino (NEt₂), dipropylamino (NPr₂) and variations thereof (eg N(Me)(Et) and so on).

The term “nitro” refers to the group —NO₂.

The term “cyano” and “nitrile” refer to the group —CN.

The term “amido” or “amide” refers to the group —C(O) NH₂.

The term “substituted amido” or “substituted amide” refers to an amido group having a hydrogen replaced with, for example a C₁-C₆ alkyl group (“C₁-C₆ alkylamido” or “C₁-C₆ alkylamide”), an aryl (“arylamido”), aralkyl group (“aralkylamido”) and so on. C₁-C₃ alkylamide groups are preferred, such as for example, methylamide (—C(O)NHMe), ethylamide (—C(O)NHEt) and propylamide (—C(O)NHPr) and includes reverse amides thereof (eg NHMeC(O)—, —NHEtC(O)— and —NHPrC(O)—).

The term “disubstituted amido” or “disubstituted amide” refers to an amido group having the two hydrogens replaced with, for example a C₁-C₆alkyl group (“di(C₁-C₆ alkyl)amido” or “di(C₁-C₆ alkyl)amide”), an aralkyl and alkyl group (“alkyl(aralkyl)amido”) and so on. Di(C₁-C₃ alkyl)amide groups are preferred, such as for example, dimethylamide (—C(O)NMe₂), diethylamide (—C(O)NEt₂) and dipropylamide ((—C(O)NPr₂) and variations thereof (eg C(O)N(Me)Et and so on) and includes reverse amides thereof.

The term “sulfonyl” refers to the group —SO₂H.

The term “substituted sulfonyl” refers to a sulfonyl group having the hydrogen replaced with, for example a C₁-C₆ alkyl group (“sulfonylC₁-C₆ alkyl”), an aryl (“arylsulfonyl”), an aralkyl (“aralkylsulfonyl”) and so on. Sulfonyl C₁-C₃ alkyl groups are preferred, such as for example, —SO₂Me, —SO₂Et and —SO₂Pr.

The term “sulfonylamido” or “sulfonamide” refers to the group —SO₂NH₂.

The term “substituted sulfonamido” or “substituted sulphonamide” refers to an sulfonylamido group having a hydrogen replaced with, for example a C₁-C₆ alkyl group (“sulfonylamidoC₁-C₆ alkyl”), an aryl (“arylsulfonamide”), aralkyl (“aralkylsulfonamide”) and so on. SulfonylamidoC₁-C₃ alkyl groups are preferred, such as for example, —SO₂NHMe, —SO₂NHEt and —SO₂NHPr and includes reverse sulfonamides thereof (e.g. —NHSO₂Me, —NHSO₂Et and —NHSO₂Pr).

The term “disubstituted sufonamido” or “disubstituted sulphonamide” refers to an sulfonylamido group having the two hydrogens replaced with, for example a C₁-C₆ alkyl group, which may be the same or different (“sulfonylamidodi(C₁-C₆ alkyl)”), an aralkyl and alkyl group (“sulfonamido(aralkyl)alkyl”) and so on. Sulfonylamidodi(C₁-C₃ alkyl) groups are preferred, such as for example, —SO₂NMe₂, —SO₂NEt₂ and —SO₂NPr₂ and variations thereof (eg —SO₂N (Me) Et and so on) and includes reserve sulfonamides thereof (eg —N(Me) SO₂Me and so on).

The term “sulfate” refers to the group OS(O)₂OH and includes groups having the hydrogen replaced with, for example a C₁-C₆ alkyl group (“alkylsulfates”), an aryl (“arylsulfate”), an aralkyl (“aralkylsulfate”) and so on. C₁-C₃ alkylsulfates are preferred, such as for example, OS(O)₂OMe, OS(O)₂OEt and OS(O)₂OPr.

The term “sulfonate” refers to the group SO₃H and includes groups having the hydrogen replaced with, for example a C₁-C₆ alkyl group (“alkylsulfonate”), an aryl (“arylsulfonate”), an aralkyl (“aralkylsulfonate”) and so on. C₁-C₃ alkylsulfonates are preferred, such as for example, SO₃Me, SO₃Et and SO₃Pr.

The term “amino acid” as herein defined refers to a moiety containing an amino group and a carboxyl group linked by at least one carbon. An amino acid may refer a natural or non-natural amino acid, preferably a natural amino acid such as alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, preferably the amino acid is arginine, lysine or histidine, most preferably lysine.

The term “carboxylate” or “carboxyl” refers to the group —COO— or —COOH.

The term “carbamate” or “carbomyl” refers to the group —OC(O)NH₂. The carbamate may be substituted, or may be disubstituted, for example with an alkyl group such as but not limited to C₁-C₆ alkyl.

The term “carbonate” refers to the group —OC(O)O— or —OC(O)OH.

The term “alkylcarbonate” as herein defined refers to a carbonate group having the hydrogen replaced with, for example a C₁-C₆ alkyl group, an aryl or aralkyl group (“arylcarbonate” or “aralkylcarbonate”) and so on. CO₃C₁-C₃alkyl groups are preferred, such as for example, methylcarbonate (CO₃Me), ethylcarbonate (CO₃Et) and propylcarbonate (CO₃Pr).

The term “ester” refers to a carboxyl group having the hydrogen replaced with, for example a C₁-C₆ alkyl group (“carboxylC₁-C₆ alkyl” or “alkylester”), an aryl or aralkyl group (“arylester” or “aralkylester”) and so on. CO₂C₁-C₃ alkyl groups are preferred, such as for example, methylester (CO₂Me), ethylester (CO₂Et) and propylester (CO₂Pr) and includes reverse esters thereof (eg —OC(O)Me, —OC(O)Et and —OC(O)Pr).

The term “heterocyclyl” refers to a moiety obtained by removing a hydrogen atom from a ring atom of a heterocyclic compound which moiety has from 3 to 12 ring atoms (unless otherwise specified), of which 1, 2, 3, 4 or more are ring heteroatoms, for example independently selected from O, S and N, or ring heteromoieties, for example independently selected from O, S, S(O), SO₂, N and NH. When a heterocyclyl group contains the prefix Cₙ₁₋ₙ₂ or “n1 to n2” this prefix indicates the number of carbon atoms in the corresponding carbocyclic group, in which one or more, suitably 1, 2, 3, 4 or more, of the ring atoms is replaced with a heteroatom or heteromoiety.

In this context, the prefixes 3-, 4-, 5-, 6-, 7-, 8-, 9- and 10-membered denote the number of ring atoms, or range of ring atoms, whether carbon atoms or heteroatoms. For example, the term “C₃₋₁₀ heterocyclyl” or “3-10 membered heterocylyl”, as used herein, pertains to a heterocyclyl group having 3, 4, 5, 6, 7, 8, 9 or 10 ring atoms. Examples of heterocylyl groups include 5-6-membered monocyclic heterocyclyls and 9-10 membered fused bicyclic heterocyclyls.

Examples of monocyclic heterocyclyl groups include, but are not limited to, those containing one nitrogen atom such as aziridine (3-membered ring), azetidine (4-membered ring), pyrrolidine (tetrahydropyrrole), pyrroline (eg 3-pyrroline, 2,5-dihydropyrrole), 2H-pyrrole or 3H-pyrrole (isopyrrole, isoazole) or pyrrolidinone (5-membered rings), piperidine, dihydropyridine, tetrahydropyridine (6-membered rings), and azepine (7-membered ring); those containing two nitrogen atoms such as imidazoline, pyrazolidine (diazolidine), imidazoline, pyrazoline (dihydropyrazole) (5-membered rings), piperazine (6-membered ring); those containing one oxygen atom such as oxirane (3-membered ring), oxetane (4-membered ring), oxolane (tetrahydrofuran), oxole (dihydrofuran) (5-membered rings), oxane (tetrahydropyran), dihydropyran, pyran (6-membered rings), oxepin (7-membered ring); those containing two oxygen atoms such as dioxolane (5-membered ring), dioxane (6-membered ring), and dioxepane (7-membered ring); those containing three oxygen atoms such as trioxane (6-membered ring); those containing one sulfur atom such as thiirane (3-membered ring), thietane (4-membered ring), thiolane (tetrahydrothiophene) (5-membered ring), thiane (tetrahydrothiopyran) (6-membered ring), thiepane (7-membered ring); those containing one nitrogen and one oxygen atom such as tetrahydrooxazole, dihydrooxazole, tetrahydroisoxazole, dihydroisoxazole (5-membered rings), morpholine, tetrahydrooxazine, dihydrooxazine, oxazine (6-membered rings); those containing one nitrogen and one sulfur atom such as thiazoline, thiazolidine (5-membered rings), thiomorpholine (6-membered ring); those containing two nitrogen and one oxygen atom such as oxadiazine (6-membered ring); those containing one oxygen and one sulfur such as: oxathiole (5-membered ring) and oxathiane (thioxane) (6-membered ring); and those containing one nitrogen, one oxygen and one sulfur atom such as oxathiazine (6-membered ring).

Heterocyclyls also encompass heteroaryl (aromatic heterocyclyls) and heterocycloalkyl (non-aromatic heterocyclyls). Such groups may be substituted or unsubstituted.

The term “aromatic heterocyclyl” may be used interchangeably with the term “heteroaromatic” or the term “heteroaryl” or “hetaryl”. The heteroatoms in the aromatic heterocyclyl group may be independently selected from N, S and O. The aromatic heterocyclyl groups may comprise 1, 2, 3, 4 or more ring heteroatoms.

When a heteroaryl group contains the prefix Cₙ₁₋ₙ₂ or “n1 to n2” this prefix indicates the number of carbon atoms in the corresponding aryl group, in which one or more, suitably 1, 2, 3, 4 or more, of the ring atoms is replaced with a heteroatom. In the case of fused aromatic heterocyclyl groups, only one of the rings may contain a heteroatom and not all rings must be aromatic.

“Heteroaryl” is used herein to denote a heterocyclic group having aromatic character and embraces aromatic monocyclic ring systems and polycyclic (eg bicyclic) ring systems containing one or more aromatic rings. The term aromatic heterocyclyl also encompasses pseudoaromatic heterocyclyls. The term “pseudoaromatic” refers to a ring system which is not strictly aromatic, but which is stabilized by means of delocalization of electrons and behaves in a similar manner to aromatic rings. The term aromatic heterocyclyl therefore covers polycyclic ring systems in which all of the fused rings are aromatic as well as ring systems where one or more rings are non-aromatic, provided that at least one ring is aromatic. In polycyclic systems containing both aromatic and non-aromatic rings fused together, the group may be attached to another moiety by the aromatic ring or by a non-aromatic ring.

Examples of heteroaryl groups are monocyclic and bicyclic groups containing from five to ten ring members. The heteroaryl group can be, for example, a five membered or six membered monocyclic ring or a bicyclic structure formed from fused five and six membered rings or two fused six membered rings or two fused five membered rings. Each ring may contain up to about four heteroatoms typically selected from nitrogen, sulphur and oxygen. The heteroaryl ring will contain up to 4 heteroatoms, more typically up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Aromatic heterocyclyl groups may be 5-membered or 6-membered mono-cyclic aromatic ring systems.

Examples of 5-membered monocyclic heteroaryl groups include but are not limited to furanyl, thienyl, pyrrolyl, oxazolyl, oxadiazolyl (including 1,2,3 and 1,2,4 oxadiazolyls and furazanyl i.e. 1,2,5-oxadiazolyl), thiazolyl, isoxazolyl, isothiazolyl, pyrazolyl, imidazolyl, triazolyl (including 1,2,3, 1,2,4 and 1,3,4 triazolyls), oxatriazolyl, tetrazolyl, thiadiazolyl (including 1,2,3 and 1,3,4 thiadiazolyls) and the like.

Examples of 6-membered monocyclic heteroaryl groups include but are not limited to pyridinyl, pyrimidinyl, pyridazinyl, pyrazinyl, triazinyl, pyranyl, oxazinyl, dioxinyl, thiazinyl, thiadiazinyl and the like. Examples of 6-membered aromatic heterocyclyls containing nitrogen include pyridyl (1 nitrogen), pyrazinyl, pyrimidinyl and pyridazinyl (2 nitrogens).

Aromatic heterocyclyl groups may also be bicyclic or polycyclic heteroaromatic ring systems such as fused ring systems (including purine, pteridinyl, napthyridinyl, 1H thieno[2,3-c]pyrazolyl, thieno[2,3-b]furyl and the like) or linked ring systems (such as oligothiophene, polypyrrole and the like). Fused ring systems may also include aromatic 5-membered or 6-membered heterocyclyls fused to carbocyclic aromatic rings such as phenyl, napthyl, indenyl, azulenyl, fluorenyl, anthracenyl and the like, such as 5-membered aromatic heterocyclyls containing nitrogen fused to phenyl rings, 5-membered aromatic heterocyclyls containing 1 or 2 nitrogens fused to phenyl ring.

A bicyclic heteroaryl group may be, for example, a group selected from: a) a benzene ring fused to a 5- or 6-membered ring containing 1, 2 or 3 ring heteroatoms; b) a pyridine ring fused to a 5- or 6-membered ring containing 1, 2 or 3 ring heteroatoms; c) a pyrimidine ring fused to a 5- or 6-membered ring containing 1 or 2 ring heteroatoms; d) a pyrrole ring fused to a 5- or 6-membered ring containing 1, 2 or 3 ring heteroatoms; e) a pyrazole ring fused to a 5- or 6-membered ring containing 1 or 2 ring heteroatoms; f) an imidazole ring fused to a 5- or 6-membered ring containing 1 or 2 ring heteroatoms; g) an oxazole ring fused to a 5- or 6-membered ring containing 1 or 2 ring heteroatoms; h) an isoxazole ring fused to a 5- or 6-membered ring containing 1 or 2 ring heteroatoms; i) a thiazole ring fused to a 5- or 6-membered ring containing 1 or 2 ring heteroatoms; j) an isothiazole ring fused to a 5- or 6-membered ring containing 1 or 2 ring heteroatoms; k) a thiophene ring fused to a 5- or 6-membered ring containing 1, 2 or 3 ring heteroatoms; I) a furan ring fused to a 5- or 6-membered ring containing 1, 2 or 3 ring heteroatoms; m) a cyclohexyl ring fused to a 5- or 6-membered ring containing 1, 2 or 3 ring heteroatoms; and n) a cyclopentyl ring fused to a 5- or 6-membered ring containing 1, 2 or 3 ring heteroatoms.

Particular examples of bicyclic heteroaryl groups containing a five membered ring fused to another five membered ring include but are not limited to imidazothiazole (e.g. imidazo[2,1-b]thiazole) and imidazoimidazole (e.g. imidazo[1,2-a]imidazole).

Particular examples of bicyclic heteroaryl groups containing a six membered ring fused to a five membered ring include but are not limited to benzofuran, benzothiophene, benzimidazole, benzoxazole, isobenzoxazole, benzisoxazole, benzothiazole, benzisothiazole, isobenzofuran, indole, isoindole, indolizine, indoline, isoindoline, purine (e.g., adenine, guanine), indazole, pyrazolopyrimidine (e.g. pyrazolo[1,5-a]pyrimidine), benzodioxole and pyrazolopyridine (e.g. pyrazolo[1,5-a]pyridine) groups. A further example of a six membered ring fused to a five membered ring is a pyrrolopyridine group such as a pyrrolo[2,3-b]pyridine group.

Particular examples of bicyclic heteroaryl groups containing two fused six membered rings include but are not limited to quinoline, isoquinoline, chroman, thiochroman, chromene, isochromene, isochroman, benzodioxan, quinolizine, benzoxazine, benzodiazine, pyridopyridine, quinoxaline, quinazoline, cinnoline, phthalazine, naphthyridine and pteridine groups.

Examples of heteroaryl groups containing an aromatic ring and a non-aromatic ring include tetrahydronaphthalene, tetrahydroisoquinoline, tetrahydroquinoline, dihydrobenzothiophene, dihydrobenzofuran, 2,3-dihydro-benzo[1,4]dioxine, benzo[1,3]dioxole, 4,5,6,7-tetrahydrobenzofuran, indoiine, isoindoline and indane groups.

Examples of aromatic heterocyclyls fused to carbocyclic aromatic rings may therefore include but are not limited to benzothiophenyl, indolyl, isoindolyl, benzofuranyl, isobenzofuranyl, benzimidazolyl, indazolyl, benzoxazolyl, benzisoxazolyl, isobenzoxazoyl, benzothiazolyl, benzisothiazolyl, quinolinyl, isoquinolinyl, quinoxalinyl, quinazolinyl, cinnolinyl, benzotriazinyl, phthalazinyl, carbolinyl and the like.

The term “heterocycloalkyl” or “non-aromatic heterocyclyl” encompasses optionally substituted saturated and unsaturated rings which contain at least one heteroatom such as N, S and O, or a heteromoiety such as O, S, S(O), SO₂, N and NH. The ring may contain 1, 2, 3, 4 or more heteroatoms or heteromoieties. When a heterocycloalkyl group contains the prefix Cₙ₁₋ₙ₂ or “n1 to n2” this prefix indicates the number of carbon atoms in the corresponding carbocyclic group, in which one or more, suitably 1, 2, 3, 4 or more, of the ring atoms is replaced with a heteroatom or heteromoiety. The ring may be a monocyclic ring or part of a polycyclic ring system. Polycyclic ring systems include fused rings and spirocycles. Not every ring in a non-aromatic heterocyclic polycyclic ring system must contain a heteroatom, provided at least one ring contains one or more heteroatoms.

Non-aromatic heterocyclyls may be 3-8 membered mono-cyclic rings.

Examples of 5-membered non-aromatic heterocyclyl rings include 2H-pyrrolyl, 1-pyrrolinyl, 2-pyrrolinyl, 3-pyrrolinyl, pyrrolidinyl, 1-pyrrolidinyl, 2-pyrrolidinyl, 3-pyrrolidinyl, tetrahydrofuranyl, tetrahydrothiophenyl, pyrazolinyl, 2-pyrazolinyl, 3-pyrazolinyl, pyrazolidinyl, 2-pyrazolidinyl, 3-pyrazolidinyl, imidazolidinyl, 3-dioxalanyl, thiazolidinyl, isoxazolidinyl, 2-imidazolinyl and the like.

Examples of 6-membered non-aromatic heterocyclyls include piperidinyl, piperidinonyl, pyranyl, dihyrdopyranyl, tetrahydropyranyl, 2H pyranyl, 4H pyranyl, thianyl, thianyl oxide, thianyl dioxide, piperazinyl, diozanyl, 1,4-dioxinyl, 1,4-dithianyl, 1,3,5-triozalanyl, 1,3,5-trithianyl, 1,4-morpholinyl, thiomorpholinyl, 1,4-oxathianyl, triazinyl, 1,4-thiazinyl and the like.

Examples of 7-membered non-aromatic heterocyclyls include azepanyl, oxepanyl, thiepanyl and the like.

Non-aromatic heterocyclyl rings may also be bicyclic heterocyclyl rings such as linked ring systems (for example uridinyl and the like) or fused ring systems. Fused ring systems include non-aromatic 5-membered, 6-membered or 7-membered heterocyclyls fused to carbocyclic aromatic rings such as phenyl, napthyl, indenyl, azulenyl, fluorenyl, anthracenyl and the like. Examples of non-aromatic 5-membered, 6-membered or 7-membered heterocyclyls fused to carbocyclic aromatic rings include indolinyl, benzodiazepinyl, benzazepinyl, dihydrobenzofuranyl and the like.

The term “alkyleneheteroaryl” refers to a radical having an alkyl component and a heteroaryl component, where the alkyl component links the heteroaryl component to the point of attachment. The alkyl component is as defined above, except that the alkyl component is at least divalent, an alkylene, to link to the heteroaryl component and to the point of attachment. In some instances, the alkyl component can be absent. The alkyl component can include any number of carbons, such as C₁₋₆, C₁₋₂, C₁₋₃, C₁₋₄, C₁₋₅, C₂₋₃, C₂₋₄, C₂₋₅, C₂₋₆, C₃₋₄, C₃₋₅, C₃₋₆, C₄₋₅, C₄₋₆ and C₅₋₆. The heteroaryl component is as defined herein. The numerical range from x to y in “C_{x-y} alkylenecycloalkyl” relates to the total number of alkyl carbons and heteroaryl ring atoms (carbon and heteroatoms together).

The term “alkylene heterocycloalkyl” refers to a radical having an alkyl component and a heterocycloalkyl component, where the alkyl component links the heterocycloalkyl component to the point of attachment. The alkyl component is as defined above, except that the alkyl component is at least divalent, an alkylene, to link to the heterocycloalkyl component and to the point of attachment. In some instances, the alkyl component can be absent. The alkyl component can include any number of carbons, such as C₁₋₆, C₁₋₂, C₁₋₃, C₁₋₄, C₁₋₅, C₂₋₃, C₂₋₄, C₂₋₅, C₂₋₆, C₃₋₄, C₃₋₅, C₃₋₆, C₄₋₅, C₄₋₆ and C₅₋₆. The heterocycloalkyl component is as defined herein. The numerical range from x to y in “C_{x-y} alkyleneheterocycloalkyl” relates to the total number of alkyl carbons and heterocycloalkyl ring atoms (carbon and heteroatoms together).

As used herein, the term solvate refers to a complex of the compound and either stoichiometric or non-stoichiometric amounts of a solvent. Solvates are often formed during the process of crystallization with pharmaceutically acceptable solvents such as water, ethanol, and the like. Hydrates are formed when the solvent is water, or alcoholates are formed when the solvent is alcohol.

As used herein, the term polymorph refers to the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

As used herein, the term “metabolite” refers to a derivative of a compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. Metabolites of the compounds disclosed herein are optionally identified either by administration of compounds to a host and analysis of tissue samples from the host, or by incubation of compounds with hepatic cells in vitro and analysis of the resulting compounds.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. The term “stereoisomers” refers to compounds which have identical chemical constitution, but differ with regard to the arrangement of the atoms or groups in space. As used herein, the term “stereoisomer” includes but is not limited to diastereomers, enantiomers and atropisomers, as well as mixtures thereof such as racemic mixtures.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

### Forms of the Compound

In the case of compounds that are solids, it will be understood by those skilled in the art that the inventive compounds, agents and salts may exist in different crystalline or polymorphic forms, all of which are intended to be within the scope of the present invention and specified formulae.

The invention includes all crystalline forms of a compound of Formula (I) including anhydrous crystalline forms, hydrates, solvates and mixed solvates. If any of these crystalline forms demonstrates polymorphism, all polymorphs are within the scope of this invention.

Formula (I) is intended to cover, where applicable, solvated as well as unsolvated forms of the compounds. Thus, Formula (I) includes compounds having the indicated structures, including the hydrated or solvated forms, as well as the non-hydrated and non-solvated forms.

The compounds of Formula (I) or salts, tautomers, N-oxides, polymorphs or prodrugs thereof may be provided in the form of solvates. Solvates contain either stoichiometric or non-stoichiometric amounts of a solvent, and may be formed during the process of crystallization with pharmaceutically acceptable solvents such as water, alcohols such as methanol, ethanol or isopropyl alcohol, DMSO, acetonitrile, dimethyl formamide (DMF), acetic acid, and the like with the solvate forming part of the crystal lattice by either non-covalent binding or by occupying a hole in the crystal lattice. Hydrates are formed when the solvent is water, alcoholates are formed when the solvent is alcohol. Solvates of the compounds of the present invention can be conveniently prepared or formed during the processes described herein. In general, the solvated forms are considered equivalent to the unsolvated forms for the purposes of the invention.

Basic nitrogen-containing groups may be quarternised with such agents as C₁₋₆alkyl halide, such as methyl, ethyl, propyl, and butyl chlorides, bromides and iodides; dialkyl sulfates like dimethyl and diethyl sulfate; and others.

Nitrogen containing groups may also be oxidised to form an N-oxide.

The compound of Formula (I) or salts, tautomers, N-oxides, solvates and/or prodrugs thereof that form crystalline solids may demonstrate polymorphism. All polymorphic forms of the compounds, salts, tautomers, N-oxides, solvates and/or prodrugs are within the scope of the invention.

The compound of Formula (I) may demonstrate tautomerism. Tautomers are two interchangeable forms of a molecule that typically exist within an equilibrium. Any tautomers of the compounds of Formula (I) are to be understood as being within the scope of the invention.

The compound of Formula (I) may contain one or more stereocentres. All stereoisomers of the compounds of formula (I) are within the scope of the invention. Stereoisomers include enantiomers, diastereomers, geometric isomers (E and Z olephinic forms and cis and trans substitution patterns) and atropisomers. In some embodiments, the compound is a stereoisomerically enriched form of the compound of formula (I) at any stereocentre. The compound may be enriched in one stereoisomer over another by at least about 60, 70, 80, 90, 95, 98 or 99%.

The compound of Formula (I) or its salts, tautomers, solvates, N-oxides, and/or stereoisomers, may be isotopically enriched with one or more of the isotopes of the atoms present in the compound. For example, the compound may be enriched with one or more of the following minor isotopes: ²H, ³H, ¹³C, ¹⁴C, ¹⁵N and/or ¹⁷O, preferably ²H. An isotope may be considered enriched when its abundance is greater than its natural abundance.

A “prodrug” is a compound that may not fully satisfy the structural requirements of the compounds provided herein, but is modified in vivo, following administration to a subject or patient, to produce a compound of formula (I) provided herein. For example, a prodrug may be an acylated derivative of a compound as provided herein. Prodrugs include compounds wherein hydroxy, carboxy, amine or sulfhydryl groups are bonded to any group that, when administered to a mammalian subject, cleaves to form a free hydroxy, carboxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to generate the parent compounds.

Prodrugs include compounds wherein an amino acid residue, or a polypeptide chain of two or more (eg, two, three or four) amino acid residues which are covalently joined to free amino, and amido groups of compounds of Formula (I). The amino acid residues include the 20 naturally occurring amino acids commonly designated by three letter symbols and also include, 4-hydroxyproline, hydroxylysine, demosine, isodemosine, 3-methylhistidine, norvlin, beta-alanine, gamma-aminobutyric acid, citrulline, homocysteine, homoserine, ornithine and methionine sulfone. Prodrugs also include compounds wherein carbonates, carbamates, amides and alkyl esters which are covalently bonded to the above substituents of Formula (I) through the carbonyl carbon prodrug sidechain.

### Compositions, Formulations and Modes of Administration

Any compound described herein may be for use as a medicament. Accordingly, the compound may be for use in treating any indication disclosed herein.

The compounds of formula (I) can be administered alone or in the form of a pharmaceutical composition. In practice, the compounds of formula (I) are usually administered in the form of pharmaceutical compositions, that is, in admixture with at least one pharmaceutically acceptable excipient. The proportion and nature of any pharmaceutically acceptable excipient(s) are determined by the properties of the selected compound of the invention, the chosen route of administration, and standard pharmaceutical practice.

In another embodiment, there is provided a pharmaceutical composition comprising a compound of formula (I) or a pharmaceutically acceptable salt, stereoisomer, solvate, metabolite, or polymorph thereof, and at least one pharmaceutically acceptable excipient.

Pharmaceutical compositions of the disclosure typically include a therapeutically effective amount of one or more active ingredients in admixture with one or more pharmaceutically and physiologically acceptable formulation materials. Suitable formulation materials include, but are not limited to, antioxidants, preservatives, coloring, flavoring and diluting agents, emulsifying agents, suspending agents, solvents, fillers, bulking agents, buffers, delivery vehicles, diluents, excipients and/or pharmaceutical adjuvants. For example, a suitable vehicle may be water for injection, physiological saline solution, or artificial perilymph, possibly supplemented with other materials common in compositions for parenteral administration. Neutral buffered saline or saline mixed with serum albumin are further exemplary vehicles.

Pharmaceutical compositions of the present disclosure additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this disclosure. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminium hydroxide; alginic acid; pyrogenfree water; isotonic saline; Ringer's solution; ethyl alcohol, and phosphate buffer solutions, as well as other non-toxic compatible lubricants such as sodium lauryl sulfate and magnesium stearate, as well as colouring agents, releasing agents, coating agents, sweetening, flavouring and perfuming agents, preservatives and antioxidants can also be present in the composition, according to the judgment of the formulator.

Various dosage units are each preferably provided as a discrete dosage tablet, capsules, lozenge, dragee, gum, or other type of solid formulation. Capsules may encapsulate a powder, liquid, or gel. The solid formulation may be swallowed, or may be of a suckable or chewable type (either frangible or gum-like). The present invention contemplates dosage unit retaining devices other than blister packs; for example, packages such as bottles, tubes, canisters, packets. The dosage units may further include conventional excipients well-known in pharmaceutical formulation practice, such as binding agents, gellants, fillers, tableting lubricants, disintegrants, surfactants, and colorants; and for suckable or chewable formulations.

A compound of formula (I) may be administered in any form and route which makes the compound bioavailable.

Compositions described herein may be administered systemically or directly to the site of condition or disease.

Compositions described herein may be formulated from compounds according to Formula (I) for any appropriate route of administration including, for example, oral, rectal, nasal, vaginal, topical (including transdermal, buccal, ocular and sublingual), parenteral (including subcutaneous, intraperitoneal, intradermal, intravascular (for example, intravenous), intramuscular, spinal, intracranial, intrathecal, intraocular, periocular, intraorbital, intrasynovial and intraperitoneal injection, intracisternal injection as well as any other similar injection or infusion techniques), inhalation, insufflation, infusion or implantation techniques (e.g., as sterile injectable aqueous or non-aqueous solutions or suspensions). In some embodiments, compositions described herein may be administered orally, nasally, intravenously, intramuscularly, topically, subcutaneously, rectally, vaginally or by urethral application.

Compositions intended for oral use may further comprise one or more components such as sweetening agents, flavouring agents, colouring agents and/or preserving agents in order to provide appealing and palatable preparations. Tablets contain the active ingredient in admixture with physiologically acceptable excipients that are suitable for the manufacture of tablets. Such excipients include, for example, inert diluents such as calcium carbonate, sodium carbonate, lactose, calcium phosphate or sodium phosphate, granulating and disintegrating agents such as corn starch or alginic acid, binding agents such as starch, gelatine or acacia, and lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatine capsules wherein the active ingredient is mixed with an inert solid diluent such as calcium carbonate, calcium phosphate or kaolin, or as soft gelatine capsules wherein the active ingredient is mixed with water or an oil medium such as peanut oil, liquid paraffin or olive oil.

Oily suspensions may be formulated by suspending the active ingredients in a vegetable oil such as arachis oil, olive oil, sesame oil or coconut oil, or in a mineral oil such as liquid paraffin. The oily suspensions may contain a thickening agent such as beeswax, hard paraffin or cetyl alcohol. Sweetening agents such as those set forth above, and/or flavouring agents may be added to provide palatable oral preparations. Such suspensions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, such as sweetening, flavouring and colouring agents, may also be present.

Pharmaceutical compositions may also be in the form of oil-in-water emulsions. The oily phase may be a vegetable oil such as olive oil or arachis oil, a mineral oil such as liquid paraffin, or a mixture thereof. Suitable emulsifying agents include naturally-occurring gums such as gum acacia or gum tragacanth, naturally-occurring phosphatides such as soy bean lecithin, and esters or partial esters derived from fatty acids and hexitol, anhydrides such as sorbitan monoleate, and condensation products of partial esters derived from fatty acids and hexitol with ethylene oxide such as polyoxyethylene sorbitan monoleate. An emulsion may also comprise one or more sweetening and/or flavouring agents.

Syrups and elixirs may be formulated with sweetening agents, such as glycerol, propylene glycol, sorbitol or sucrose. Such Formulations may also comprise one or more demulcents, preservatives, flavouring agents and/or colouring agents.

A composition may further include one or more components adapted to improve the stability or effectiveness of the applied formulation, such as stabilizing agents, suspending agents, emulsifying agents, viscosity adjusters, gelling agents, preservatives, antioxidants, skin penetration enhancers, moisturizers and sustained release materials. Examples of such components are described in Martindale—The Extra Pharmacopoeia (Pharmaceutical Press, London 1993) and Martin (ed.), Remington's Pharmaceutical Sciences. Formulations may comprise microcapsules, such as hydroxymethylcellulose or gelatine-microcapsules, liposomes, albumin microspheres, microemulsions, nanoparticles or nanocapsules.

Preservatives include, but are not limited to, antimicrobials such as methylparaben, propylparaben, sorbic acid, benzoic acid, and formaldehyde, as well as physical stabilizers and antioxidants such as vitamin E, sodium ascorbate/ascorbic acid and propyl gallate. Suitable moisturizers include, but are not limited to, lactic acid and other hydroxy acids and their salts, glycerine, propylene glycol, and butylene glycol. Suitable emollients include lanolin alcohol, lanolin, lanolin derivatives, cholesterol, petrolatum, isostearyl neopentanoate and mineral oils. Suitable fragrances and colours include, but are not limited to, FD&C Red No. 40 and FD&C Yellow No. 5. Other suitable additional ingredients that may be included in a topical Formulation include, but are not limited to, abrasives, absorbents, anticaking agents, antifoaming agents, antistatic agents, astringents (such as witch hazel), alcohol and herbal extracts such as chamomile extract, binders/excipients, buffering agents, chelating agents, film forming agents, conditioning agents, propellants, opacifying agents, pH adjusters and protectants.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S. P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

A pharmaceutical composition may be formulated as inhaled formulations, including sprays, mists, or aerosols. For inhalation formulations, the composition or combination provided herein may be delivered via any inhalation methods known to a person skilled in the art. Such inhalation methods and devices include, but are not limited to, metered dose inhalers with propellants such as CFC or HFA or propellants that are physiologically and environmentally acceptable. Other suitable devices are breath operated inhalers, multidose dry powder inhalers and aerosol nebulizers. Aerosol formulations for use in the subject method typically include propellants, surfactants and co-solvents and may be filled into conventional aerosol containers that are closed by a suitable metering valve.

Inhalant compositions may comprise liquid or powdered compositions containing the active ingredient that are suitable for nebulization and intrabronchial use, or aerosol compositions administered via an aerosol unit dispensing metered doses. Suitable liquid compositions comprise the active ingredient in an aqueous, pharmaceutically acceptable inhalant solvent such as isotonic saline or bacteriostatic water. The solutions are administered by means of a pump or squeeze-actuated nebulized spray dispenser, or by any other conventional means for causing or enabling the requisite dosage amount of the liquid composition to be inhaled into the patient's lungs. Suitable Formulations, wherein the carrier is a liquid, for administration, as for example, a nasal spray or as nasal drops, include aqueous or oily solutions of the active ingredient.

Compositions suitable for rectal administration are preferably presented as unit dose suppositories. These may be prepared by at least partially dispersing the active in one or more lipophilic bases and then shaping the mixture.

Pharmaceutical compositions may be formulated as sustained release formulations such as a capsule that creates a slow release of active following administration. Such formulations may generally be prepared using well-known technology and administered by, for example, oral, rectal or subcutaneous implantation, or by implantation at the desired target site. Carriers for use within such formulations are biocompatible, and may also be biodegradable. Preferably, the formulation provides a relatively constant level of active release. The amount of active contained within a sustained release formulation depends upon, for example, the site of implantation, the rate and expected duration of release and the nature of the condition to be treated.

One skilled in the art can readily select the proper form and route of administration depending on the particular characteristics of the compound selected, the disease or condition to be treated, the stage of the disease or condition, and other relevant circumstances.

It will be understood, that the specific dose level for any particular patient will depend upon a variety of factors including the activity of the specific compound employed, the age, body weight, general health, sex, diet, time of administration, route of administration, number of doses, and rate of excretion, drug combination (i.e. other drugs being used to treat the patient), and the severity of the particular disorder undergoing therapy.

The phrase “therapeutically effective amount” generally refers to an amount of one or more active ingredients of the invention that (i) treats the particular disease, condition, or disorder, (ii) attenuates, ameliorates, or eliminates one or more sign or symptoms of the particular disease, condition, or disorder, or (iii) delays the onset of one or more sign or symptoms of the particular disease, condition, or disorder described herein.

Typically, a therapeutically effective dosage is formulated to contain a concentration (by weight) of at least about 0.1% up to about 50% or more, and all combinations and sub-combinations of ranges therein. The compositions can be formulated to contain one or more actives described herein in a concentration of from about 0.1 to less than about 50%, for example, about 49, 48, 47, 46, 45, 44, 43, 42, 41 or 40%, with concentrations of from greater than about 0.1%, for example, about 0.2, 0.3, 0.4 or 0.5%, to less than about 40%, for example, about 39, 38, 37, 36, 35, 34, 33, 32, 31 or 30%. Exemplary compositions may contain from about 0.5% to less than about 30%, for example, about 29, 28, 27, 26, 25, 25, 24, 23, 22, 21 or 20%, with concentrations of from greater than about 0.5%, for example, about 0.6, 0.7, 0.8, 0.9 or 1%, to less than about 20%, for example, about 19, 18, 17, 16, 15, 14, 13, 12, 11 or 10%. The compositions can contain from greater than about 1% for example, about 2%, to less than about 10%, for example about 9 or 8%, including concentrations of greater than about 2%, for example, about 3 or 4%, to less than about 8%, for example, about 7 or 6%. The active agent can, for example, be present in a concentration of about 5%. In all cases, amounts may be adjusted to compensate for differences in amounts of active ingredients actually delivered to the treated cells or tissue.

The frequency of administration may be once daily, 2, 3 or 4 times daily. The treatment period may be for the duration of the detectable disease.

In some embodiments, the pharmaceutical composition comprises a compound according to any one of the herein disclosed embodiments, or a pharmaceutically acceptable salt, solvate, tautomer, N-oxide, stereoisomer, metabolite, polymorph or prodrug thereof, an additional therapeutic agent, and a pharmaceutically acceptable excipient.

The additional agent may be any suitable agent described herein. In some embodiments, the additional agent is a psychoactive drug, including those described herein. In some embodiments, the additional agent is useful for treatment of a disease, disorder or condition by activation of a serotonin receptor, including those described herein. In some embodiments, the additional agent is selected from any one of the following, including those described herein: an agent for a mental illness and/or a neuropsychiatric condition; an agent for psychosis and/or psychotic symptoms; an agent for attention deficit hyperactivity disorder and/or attention deficit disorder; an agent for dementia and/or Alzheimer's disease; and an agent for an addiction disorder.

### Applications

The present disclosure provides methods of using the compounds of formula (I) and compositions as described in any one of the foregoing paragraphs. The present disclosure also provides methods of delivering to a subject in need thereof a compound of formula (I) or a composition (e.g., an effective amount of the compound or composition) of the present disclosure.

In another aspect, the present disclosure provides methods of treating a disease in a subject in need thereof comprising administering to the subject in need thereof an effective amount (e.g., therapeutically effective amount) of a compound or composition (e.g., pharmaceutical composition) of the present disclosure.

In another aspect, the present disclosure provides methods of preventing a disease in a subject in need thereof comprising administering to the subject in need thereof an effective amount (e.g., therapeutically effective amount) of a compound of formula (I) or composition (e.g., pharmaceutical composition) of the present disclosure.

In another aspect, provided herein are uses of the compounds of formula (I) or compositions of the present disclosure in the manufacture of a medicament for use in a method (e.g., method of delivering an active agent to a subject in need thereof, method of treating a disease in a subject in need thereof, method of preventing a disease in a subject in need thereof) of the present disclosure.

In another aspect, provided herein are uses of the compounds of formula (I) or compositions of the present disclosure in a method (e.g., method of delivering an active agent to a subject in need thereof, method of treating a disease in a subject in need thereof, method of preventing a disease in a subject in need thereof) of the present disclosure.

In certain embodiments, the effective amount is effective in treating the disease. In certain embodiments, the effective amount is effective in preventing the disease.

In another aspect, the present disclosure provides a method of treating a disease, disorder or condition by activation of a serotonin receptor, the method comprising administering to a subject in need thereof a compound of formula (I) or a pharmaceutical composition as described herein.

In another aspect, the present disclosure provides a method of preventing a disease, disorder or condition by activation of a serotonin receptor, the method comprising administering to a subject in need thereof a compound of formula (I) or a pharmaceutical composition as described herein.

In another aspect, the present disclosure provides method of treating a disease, disorder or condition by activation of a serotonin receptor, the method comprising administering to a subject in need thereof a compound of formula (I) or a pharmaceutical composition as described herein, in combination with another known agent useful for treatment of a disease, disorder or condition by activation of a serotonin receptor. The other known agents useful for treatment of a disease, disorder or condition by activation of a serotonin receptor may be any suitable agents known in the art, including those described herein.

In another aspect, the present disclosure provides method of preventing a disease, disorder or condition by activation of a serotonin receptor, the method comprising administering to a subject in need thereof a compound of formula (I) or a pharmaceutical composition as described herein, in combination with another known agent useful for prevention of a disease, disorder or condition by activation of a serotonin receptor.

In certain embodiments, the serotonin receptor is 5-HT_{2A}.

In certain embodiments, the serotonin receptor is one or both of 5-HT_{2A} and 5-HT_{2C}. Additionally, or alternatively, in some embodiments, the serotonin receptor is not 5-HT_{2B}.

In some embodiments, the compound of formula (I) of the present disclosure is selective towards the 5-HT_{2A} receptor over one or both of the 5-HT_{2C} receptor and the 5-HT_{2B} receptor, preferably over the 5-HT_{2B} receptor. In some embodiments, the compound of formula (I) is selective towards the 5-HT_{2C} receptor over one or both of the 5-HT_{2A} receptor and the 5-HT_{2B} receptor, preferably over the 5-HT_{2B} receptor. In some embodiments, the compound of formula (I) is selective toward the 5-HT_{2A} receptor and 5-HT_{2C} receptor over the 5-HT_{2B} receptor.

In some embodiments, the compound of formula (I) of the present disclosure exhibits an EC₅₀ value for the 5-HT_{2A} receptor of less than about 1 mM, less than about 100 UM, less than about 10 μM, less than about 1 μM, or less than about 100 nM, or less than about 10 nM, as determined by an assay described herein, for example an assay of calcium flux activity such as measuring changes in intracellular calcium. In some embodiments, the compound of formula (I) exhibits an EC₅₀ for the 5-HT_{2A} receptor of less than about 1 mM, less than about 900 μM, less than about 800 μM, less than about 700 μM, less than about 600 μM, less than about 500 μM, less than about 400 μM, less than about 300 μM, less than about 200 μM, less than about 100 μM, less than about 90 μM, less than about 80 μM, less than about 70 μM, less than about 60 μM, less than about 50 μM, less than about 40 μM, less than about 30 μM, less than about 20 μM, less than about 10 μM, less than about 9 μM, less than about 8 μM, less than about 7 μM, less than about 6 μM, less than about 5 μM, less than about 4 μM, less than about 3 μM, less than about 2 μM, less than about 1 μM, less than about 900 nM, less than about 800 nM, less than about 700 nM, less than about 600 nM, less than about 500 nM, less than about 400 nM, less than about 300 nM, less than about 200 nM, or less than about 100 nM, or any equivalent unit of measure (e.g., mol/L), as determined by an assay of calcium flux activity.

In some embodiments, the compound of formula (I) of the present disclosure exhibits an EC₅₀ value for the 5-HT_{2C} receptor of less than about 1 mM, less than about 100 μM, less than about 10 μM, less than about 1 μM, or less than about 100 nM, or less than about 10 nM, as determined by an assay described herein, for example an assay of calcium flux activity such as measuring changes in intracellular calcium. In some embodiments, the compound of formula (I) exhibits an EC₅₀ for the 5-HT_{2C} receptor of less than about 1 mM, less than about 900 μM, less than about 800 μM, less than about 700 μM, less than about 600 μM, less than about 500 μM, less than about 400 μM, less than about 300 μM, less than about 200 μM, less than about 100 μM, less than about 90 μM, less than about 80 μM, less than about 70 μM, less than about 60 μM, less than about 50 μM, less than about 40 μM, less than about 30 μM, less than about 20 μM, less than about 10 μM, less than about 9 μM, less than about 8 μM, less than about 7 μM, less than about 6 μM, less than about 5 μM, less than about 4 μM, less than about 3 μM, less than about 2 μM, less than about 1 μM, less than about 900 nM, less than about 800 nM, less than about 700 nM, less than about 600 nM, less than about 500 nM, less than about 400 nM, less than about 300 nM, less than about 200 nM, or less than about 100 nM, or any equivalent unit of measure (e.g., mol/L), as determined by an assay of calcium flux activity.

In some embodiments, the compound of formula (I) of the present disclosure exhibits an EC₅₀ value for the 5-HT_{2B} receptor of greater than about 1 μM, greater than about 10 μM, or greater than about 100 μM, as determined by an assay described herein, for example an assay of calcium flux activity such as measuring changes in intracellular calcium.

In some embodiments, the disease, disorder or condition that is treated by activation of a serotonin receptor is a mental illness or a neuropsychiatric condition. Accordingly, the present application also includes a method of treating a mental illness or a neuropsychiatric condition comprising administering to a subject in need thereof a compound of formula (I) or a composition as described herein. The present application also includes a use of a compound of formula (I) of the present disclosure for treatment of a mental illness or a neuropsychiatric condition, as well as a use of a compound of formula (I) of the present disclosure for the preparation of a medicament for treatment of a meformulationtal illness or a neuropsychiatric condition. The application further includes a compound of formula (I) of the present disclosure for use in treating a mental illness or a neuropsychiatric condition.

In some embodiments, the disease, disorder or condition that is treated by activation of a serotonin receptor is a mental illness or a neuropsychiatric condition and compound of formula (I) of the present disclosure is administered in combination with one or more additional agents for a mental illness or a neuropsychiatric condition. The one or more additional agents for a mental illness or a neuropsychiatric condition may be any suitable agents known in the art, including those described herein. In some embodiments, the additional agents for a mental illness or a neuropsychiatric condition is selected from antipsychotics, including typical antipsychotics and atypical antipsychotics; antidepressants including selective serotonin reuptake inhibitors (SSRIs) and selective norepinephrine reuptake inhibitors (SNRIs), tricyclic antidepressants and monoamine oxidase inhibitors (MAOIs) (e.g. bupropion); anti-anxiety medication including benzodiazepines such as alprazolam; agents for an addiction disorder such as alcohol addiction (e.g., disulfiram), nicotine dependence (e.g., varenicline) and opioid use disorder (e.g., methadone, buprenorphine, buprenorphine-naloxone and buprenorphine long-acting injection); mood stabilizers such as lithium and anticonvulsants such carbamazepine, divalproex (valproic acid), lamotrigine, gabapentin and topiramate.

In some embodiments, the disease, disorder or condition that is treated by activation of a serotonin receptor is neurodegeneration. Accordingly, the present application also includes a method of treating neurodegeneration comprising administering to a subject in need thereof a compound of formula (I) or a composition as described herein. The present application also includes a use of a compound of formula (I) of the present disclosure for treatment of neurodegeneration, as well as a use of a compound of formula (I) of the present disclosure for the preparation of a medicament for treatment neurodegeneration. The application further includes a compound of formula (I) of the present disclosure for use in treating neurodegeneration. In some embodiments, the disease, disorder or condition that is treated by activation of a serotonin receptor is reduced brain-derived neurotrophic factor (BDNF), mammalian target of rapamycin (mTOR) activation and/or inflammation.

In some embodiments, the disease, disorder or condition that is treated by activation of a serotonin receptor comprises cognitive impairment; ischemia including stroke; neurodegeneration; refractory substance use disorders; sleep disorders; pain, such as social pain, acute pain, cancer pain, chronic pain, breakthrough pain, bone pain, soft tissue pain, nerve pain, referred pain, phantom pain, neuropathic pain, cluster headaches and migraine; obesity and eating disorders; epilepsies and seizure disorders; neuronal cell death; excitotoxic cell death; or a combination thereof.

In some embodiments, the disease, disorder or condition that is treated by activation of a serotonin receptor is psychosis or psychotic symptoms. Accordingly, the present application also includes a method of treating psychosis or psychotic symptoms comprising administering to a subject in need thereof a compound of formula (I) or a composition as described herein. The present application also includes a use of a compound of formula (I) of the present disclosure for treatment of psychosis or psychotic symptoms, as well as a use of a compound of formula (I) of the present disclosure for the preparation of a medicament for treatment of psychosis or psychotic symptoms. The application further includes a compound of formula (I) of the present disclosure for use in treating psychosis or psychotic symptoms.

In some embodiments, the disease, disorder or condition that is treated by activation of a serotonin receptor is psychosis or psychotic symptoms and the compound of formula (I) of the present disclosure is administered in combination with one or more additional agents for psychosis or psychotic symptoms. The one or more additional agents for psychosis or psychotic symptoms may be any suitable agents known in the art, including those described herein. In some embodiments, the additional agents for psychosis or psychotic symptoms are selected typical antipsychotics and atypical antipsychotics. The typical antipsychotics may be selected from acepromazine, acetophenazine, benperidol, bromperidol, butaperazine, carfenazine, chlorproethazine, chlorpromazine, chlorprothixene, clopenthixol, cyamemazine, dixyrazine, droperidol, fluanisone, flupentixol, fluphenazine, fluspirilene, haloperidol, levomepromazine, lenperone, loxapine, mesoridazine, metitepine, molindone, moperone, oxypertine, oxyprotepine, penfluridol, perazine, periciazine, perphenazine, pimozide, pipamperone, piperacetazine, pipotiazine, prochlorperazine, promazine, prothipendyl, spiperone, sulforidazine, thiopropazate, thioproperazine, thioridazine, thiothixene, timiperone, trifluoperazine, trifluperidol, triflupromazine and zuclopenthixol and combinations thereof. The atypical antipsychotics may be selected from amoxapine, amisulpride, aripiprazole, asenapine, blonanserin, brexpiprazole, cariprazine, carpipramine, clocapramine, clorotepine, clotiapine, clozapine, iloperidone, levosulpiride, lurasidone, melperone, mosapramine, nemonapride, olanzapine, paliperidone, perospirone, quetiapine, remoxipride, reserpine, risperidone, sertindole, sulpiride, sultopride, tiapride, veralipride, ziprasidone and zotepine, and combinations thereof.

In some embodiments, administering to said subject in need thereof a therapeutically effective amount of the compound of formula (I) of the present disclosure does not result in a worsening of psychosis or psychotic symptoms such as, but not limited to, hallucinations and delusions. In some embodiments, administering to said subject in need thereof a therapeutically effective amount of the compound of formula (I) results in an improvement of psychosis or psychotic symptoms such as, but not limited to, hallucinations and delusions. In some embodiments, administering to said subject in need thereof a therapeutically effective amount of the compounds of formula (I) results in an improvement of psychosis or psychotic symptoms.

In some embodiments, the disease, disorder or condition that is treated by activation of a serotonin receptor is a central nervous system (CNS) disease, disorder or condition and/or a neurological disease, disorder or condition. Accordingly, the present application also includes a method of treating a CNS disease, disorder or condition and/or a neurological disease, disorder or condition comprising administering a therapeutically effective amount of compound of formula (I) or a composition of the present disclosure to a subject in need thereof. The present application also includes a use of compound of formula (I) of the present disclosure for treatment a CNS disease, disorder or condition and/or a neurological disease, disorder or condition, as well as a use of compound of formula (I) of the present disclosure for the preparation of a medicament for treatment of a CNS disease, disorder or condition and/or a neurological disease, disorder or condition. The application further includes a compound of formula (I) of the present disclosure of the application for use in treating a CNS disease, disorder or condition and/or a neurological disease, disorder or condition.

In some embodiments, the disease, disorder or condition that is treated by activation of a serotonin receptor is a central nervous system (CNS) disease, disorder or condition and/or a neurological disease, disorder or condition and the compound of formula (I) of the present disclosure is administered in combination with one or more additional agents for a central nervous system (CNS) disease, disorder or condition and/or a neurological disease, disorder or condition. The one or more additional agents for a central nervous system (CNS) disease, disorder or condition and/or a neurological disease, disorder or condition may be any suitable agents known in the art, including those described herein. In some embodiments, the additional agents for a central nervous system (CNS) disease, disorder or condition and/or a neurological disease, disorder or condition are selected from lithium, olanzapine, quetiapine, risperidone, ariprazole, ziprasidone, clozapine, divalproex sodium, lamotrigine, valproic acid, carbamazepine, topiramate, levomilnacipran, duloxetine, venlafaxine, citalopram, fluvoxamine, escitalopram, fluoxetine, paroxetine, sertraline, clomipramine, amitriptyline, desipramine, imipramine, nortriptyline, phenelzine, tranylcypromine, diazepam, alprazolam, clonazepam, or any combination thereof. Non limiting examples of standard of care therapy for depression are sertraline, fluoxetine, escitalopram, venlafaxine, or aripiprazole. Non-limiting examples of standard of care therapy for depression are citralopram, escitalopram, fluoxetine, paroxetine, diazepam, or sertraline.

In some embodiments, the disease, disorder or condition that is treated by activation of a serotonin receptor is selected from attention deficit hyperactivity disorder and attention deficit disorder and a combination thereof. Accordingly, the present application also includes a method of treating attention deficit hyperactivity disorder and/or attention deficit disorder comprising administering to a subject in need thereof a compound of formula (I) or a composition as described herein. The present application also includes a use of a compound of formula (I) of the present disclosure for treatment of attention deficit hyperactivity disorder and/or attention deficit disorder, as well as a use of a compound of formula (I) of the present disclosure for the preparation of a medicament for treatment of attention deficit hyperactivity disorder and/or attention deficit disorder. The application further includes a compound of formula (I) of the present disclosure for use in treating attention deficit hyperactivity disorder and/or attention deficit disorder.

In some embodiments, the disease, disorder or condition that is treated by activation of a serotonin receptor is attention deficit hyperactivity disorder and/or attention deficit disorder and a combination thereof and the compound of formula (I) of the present disclosure is administered in combination with one or more additional agents for attention deficit hyperactivity disorder and/or attention deficit disorder and a combination thereof. The one or more additional agents for attention deficit hyperactivity disorder and/or attention deficit disorder may be any suitable agents known in the art, including those described herein. In some embodiments, the additional agents for attention deficit hyperactivity disorder and/or attention deficit disorder and a combination thereof are selected from methylphenidate, dexamphetamine, lisdexamfetine, atomoxetine and amphetamine and a combination thereof.

In some embodiments, the disease, disorder or condition that is treated by activation of a serotonin receptor is selected from dementia and Alzheimer's disease and a combination thereof. Accordingly, the present application also includes a method of treating dementia and/or Alzheimer's disease comprising administering to a subject in need thereof a compound of formula (I) or a composition as described herein. The present application also includes a use of a compound of formula (I) of the present disclosure for treatment of dementia and/or Alzheimer's disease, as well as a use of a compound of formula (I) of the present disclosure for the preparation of a medicament for treatment of dementia and/or Alzheimer's disease. The application further includes a compound of formula (I) of the present disclosure for use in treating dementia and/or Alzheimer's disease.

In some embodiments, the disease, disorder or condition that is treated by activation of a serotonin receptor is dementia or Alzheimer's disease and the compound of formula (I) of the present disclosure is administered in combination with one or more additional agents for dementia or Alzheimer's disease. The one or more additional agents for dementia or Alzheimer's disease may be any suitable agents known in the art, including those described herein. In some embodiments, the additional agents for dementia and Alzheimer's disease are selected from acetylcholinesterase inhibitors, NMDA antagonists and nicotinic agonists. The acetylcholinesterase inhibitors may be selected from donepezil, galantamine, rivastigmine, and phenserine, and combinations thereof. The NMDA antagonists may be selected from MK-801, ketamine, phencyclidine, and memantine, and combinations thereof. The nicotinic agonists may be selected from nicotine, nicotinic acid, nicotinic alpha7 agonists, or alpha2 beta4 agonists or a combination thereof.

In another aspect, the present disclosure provides a method of treating a mental illness, the method comprising administering to a subject in need thereof a compound of formula (I) or a pharmaceutical composition as described herein. In another aspect, the present disclosure provides a method of preventing a mental illness, the method comprising administering to a subject in need thereof a compound of formula (I) or a pharmaceutical composition as described herein. The mental illness may be a neuropsychiatric condition.

In certain embodiments, the mental illness is selected from anxiety disorders such as generalized anxiety disorder, panic disorder, social anxiety disorder and specific phobias; depression such as, hopelessness, loss of pleasure, fatigue and suicidal thoughts; mood disorders, such as depression, bipolar disorder, cancer-related depression, anxiety and cyclothymic disorder; psychotic disorders, such as hallucinations, delusions, mania, schizophrenia, schizoaffective disorder, schizophreniform Disorder; impulse control and addiction disorders, such as pyromania (starting fires), kleptomania (stealing) and compulsive gambling; alcohol addiction; drug addiction, such as opioid addiction/dependence, nicotine dependence, cocaine dependence, marijuana abuse and so on; smoking cessation; personality disorders, such as antisocial personality disorder, aggression, obsessive-compulsive personality disorder and paranoid personality disorder; obsessive-compulsive disorder (OCD), such as thoughts or fears that cause a subject to perform certain rituals or routines; post-traumatic stress disorder (PTSD); stress response syndromes (formerly called adjustment disorders); dissociative disorders, formerly called multiple personality disorder, or “split personality,” and depersonalization disorder; factitious disorders; sexual and gender disorders, such as sexual dysfunction, gender identity disorder and the paraphilias; somatic symptom disorders, formerly known as a psychosomatic disorder or somatoform disorder.

In certain embodiments, the mental illness is selected from hallucinations and delusions and a combination thereof. In these embodiments, the hallucinations may be selected from visual hallucinations, auditory hallucinations, olfactory hallucinations, gustatory hallucinations, tactile hallucinations, proprioceptive hallucinations, equilibrioceptive hallucinations, nociceptive hallucinations, thermoceptive hallucinations and chronoceptive hallucinations, and a combination thereof.

In another aspect, the present disclosure provides a method for treating a central nervous system (CNS) disease, disorder or condition and/or a neurological disease, disorder or condition, the method comprising administering to a subject in need thereof a compound of formula (I) or a pharmaceutical composition as described herein.

In another aspect, the present disclosure provides a method for preventing a central nervous system (CNS) disease, disorder or condition and/or a neurological disease, disorder or condition, the method comprising administering to a subject in need thereof a compound of formula (I) or a pharmaceutical composition as described herein.

In some embodiments, the CNS disease, disorder or condition and/or neurological disease, disorder or condition is selected from neurological diseases including neurodevelopmental diseases and neurodegenerative diseases such as Alzheimer's disease; presenile dementia; senile dementia; vascular dementia; Lewy body dementia; cognitive impairment, Parkinson's disease and Parkinsonian related disorders such as Parkinson dementia, corticobasal degeneration, and supranuclear palsy; epilepsy; CNS trauma; CNS infections; CNS inflammation; stroke; multiple sclerosis; Huntington's disease; mitochondrial disorders; Fragile X syndrome; Angelman syndrome; hereditary ataxias; neuro-otological and eye movement disorders; neurodegenerative diseases of the retina amyotrophic lateral sclerosis; tardive dyskinesias; hyperkinetic disorders; attention deficit hyperactivity disorder and attention deficit disorders; restless leg syndrome; Tourette's syndrome; Tic disorder; schizophrenia; autism spectrum disorders; tuberous sclerosis; Rett syndrome; cerebral palsy; disorders of the reward system including eating disorders such as anorexia nervosa and bulimia nervosa; binge eating disorder, trichotillomania, dermotillomania, nail biting; migraine; fibromyalgia; and peripheral neuropathy of any etiology, and combinations thereof.

In another aspect, the present disclosure provides a method for increasing neuronal plasticity, the method comprising contacting a neuronal cell with a compound of formula (I) or a pharmaceutical composition as described herein, in an amount sufficient to increase neuronal plasticity of the neuronal cell. “Neuronal plasticity” refers to the ability of the brain to change its structure and/or function continuously throughout a subject's life. Examples of the changes to the brain include, but are not limited to, the ability to adapt or respond to internal and/or external stimuli, such as due to an injury, and the ability to produce new neurites, dendritic spines, and synapses. Increasing neuronal plasticity includes, but is not limited to, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing neuronal plasticity comprises promoting neuronal growth, promoting neuritogenesis, promoting synaptogenesis, promoting dendritogenesis, increasing dendritic arbor complexity, and increasing dendritic spine density.

In some embodiments, increasing neuronal plasticity can treat neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In another aspect the present disclosure provides methods of treating weight, comprising administering an effective amount of a compound of the invention to a subject in need thereof. Treatment of weight may include treating weight gain; weight loss; metabolic disorder; weight gain associated with pharmaceutical intervention; weight gain associated with a mental illness (including those described herein); eating disorders such as anorexia, bulimia, cachexia, etc.; eating behaviour; obesity; diabetes; insulin resistance; pre-diabetes; glucose intolerance; hyperlipidemia; and cardiovascular disease.

In another aspect, the present disclosure provides a method for increasing dendritic spine density, the method comprising contacting a neuronal cell with a compound of formula (I) or a pharmaceutical composition as described herein, in an amount sufficient to increase dendritic spine density of the neuronal cell.

In certain embodiments, the compound of formula (I) produces a maximum number of dendritic crossings with an increase of greater than 1.0 fold by a Sholl Analysis.

In another aspect the present disclosure provides a method for activating a serotonin receptor in a cell, either in a biological sample or in a patient, comprising administering a compound of formula (I) as defined in any one of the herein disclosed embodiments to the cell. The serotonin receptor may be a 5-HT receptor subtype, preferably one or both of 5-HT_{2A} and 5-HT_{2C}.

In some embodiments, effective amounts vary according to factors such as the disease state, age, sex and/or weight of the subject or species. In some embodiments, the amount of a given compound or compounds that will correspond to an effective amount will vary depending upon factors, such as the given drug(s) or compound(s), the pharmaceutical formulation, the route of administration, the type of condition, disease or disorder, the identity of the subject being treated and the like, but can nevertheless be routinely determined by one skilled in the art.

In some embodiments, the compounds of formula (I) of the present disclosure are administered one, two, three or four times a year. In some embodiments, the compounds of the present disclosure are administered at least once a week. However, in another embodiment, the compounds are administered to the subject from about one time per two weeks, three weeks or one month. In another embodiment, the compounds are administered about one time per week to about once daily. In another embodiment, the compounds are administered 1, 2, 3, 4, 5 or 6 times daily. The length of the treatment period depends on a variety of factors, such as the severity of the disease, disorder or condition, the age of the subject, the concentration and/or the activity of the compounds of the application and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration is required. For example, the compounds are administered to the subject in an amount and for duration sufficient to treat the subject.

In some embodiments, the compounds of the application are administered at doses that are hallucinogenic or psychotomimetic and taken in conjunction with psychotherapy or therapy and may occur once, twice, three, or four times a year. However, in some embodiments, the compounds are administered to the subject once daily, once every two days, once every 3 days, once a week, once every two weeks, once a month, once every two months, or once every three months at doses that are not hallucinogenic or psychotomimetic.

A compound of formula (I) of the present disclosure may be either used alone or in combination with other known agents useful for treating diseases, disorders or conditions by activation of a serotonin receptor, such as the compounds of the present disclosure. When used in combination with other known agents useful in treating diseases, disorders by activation of a serotonin receptor, it is an embodiment that a compound of formula (I) is administered contemporaneously with those agents. As used herein, “contemporaneous administration” of two substances to a subject means providing each of the two substances so that they are both active in the individual at the same time. The exact details of the administration will depend on the pharmacokinetics of the two substances in the presence of each other and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present application that a combination of agents is administered to a subject in a non-contemporaneous fashion. In some embodiments, a compound of formula (I) of the present disclosure is administered with another therapeutic agent simultaneously or sequentially in separate unit dosage forms or together in a single unit dosage form. Accordingly, the present application provides a single unit dosage form comprising one or more compounds of formula (I) as described herein, an additional therapeutic agent and a pharmaceutically acceptable carrier.

In some embodiments, the compounds of the application are used or administered in an effective amount which comprises administration of doses or dosage regimens that are devoid of clinically meaningful psychedelic/psychotomimetic actions. In some embodiments, the compounds of the application are used or administered in an effective amount which comprises administration of doses or dosage regimens that provide clinical effects similar to those exhibited by a human plasma psilocin Cmax of 4 ng/mL or less and/or human 5-HT_{2A} human CNS receptor occupancy of 40% or less or those exhibited by a human plasma psilocin Cmax of 1 ng/ml or less and/or human 5-HT_{2A} human CNS receptor occupancy of 30% or less. In some embodiments, the compounds of the application are used or administered in an effective amount which comprises administration of doses or dosage regimens that provide clinical effects similar to those exhibited by a human plasma psilocin Tmax in excess of 60 minutes, in excess of 120 minutes or in excess of 180 minutes.

### Kit

In another embodiment there is provided a kit or article of manufacture including one or more compounds, pharmaceutically acceptable salt, stereoisomer, solvate, metabolite, or polymorph, and/or pharmaceutical compositions as described above.

In other embodiments there is provided a kit for use in a therapeutic application mentioned above, the kit including:
a container holding one or more compounds, pharmaceutically acceptable salt, stereoisomer, solvate, metabolite, or polymorph and/or pharmaceutical compositions as described herein;
a label or package insert with instructions for use.

It will be understood that the invention disclosed and defined in this specification extends to all alternative combinations of two or more of the individual features mentioned or evident from the text or drawings. All of these different combinations constitute various alternative aspects of the invention.

### EXAMPLES

Scheme 1: Compounds of general formula (I) can be synthesised from the appropriately substituted aniline following the outlined sequence of steps in Scheme 1 or similar as one skilled in the art may consider. Fischer indole synthesis utilising the appropriately substituted aniline allowed access to tetracyclic intermediates that upon subsequent ring opening and decarboxylation allowed for the formation of intermediate 5. Reductive alkylation of the pendant amine provided access to compounds of general formula (I) (Exemplified by P-1 and P-2). One skilled in the art will recognise that utilising differentially substituted amines would allow access to compounds of general formula (I) disclosed herein.

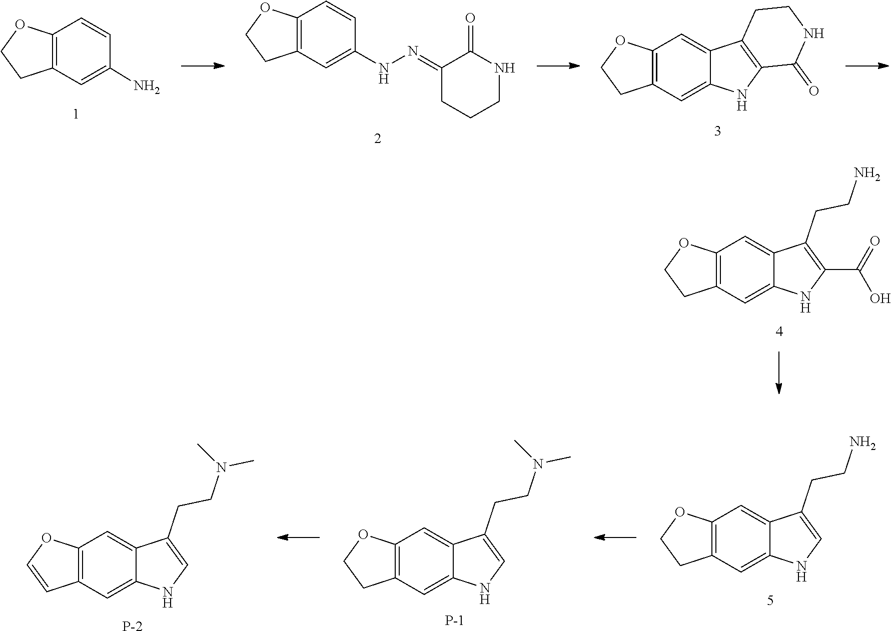

### Example 1: 2-(1,6-dihydro-2H-furo[3,2-e]indol-8-yl)-N,N-dimethylethan-1-amine (P-1)

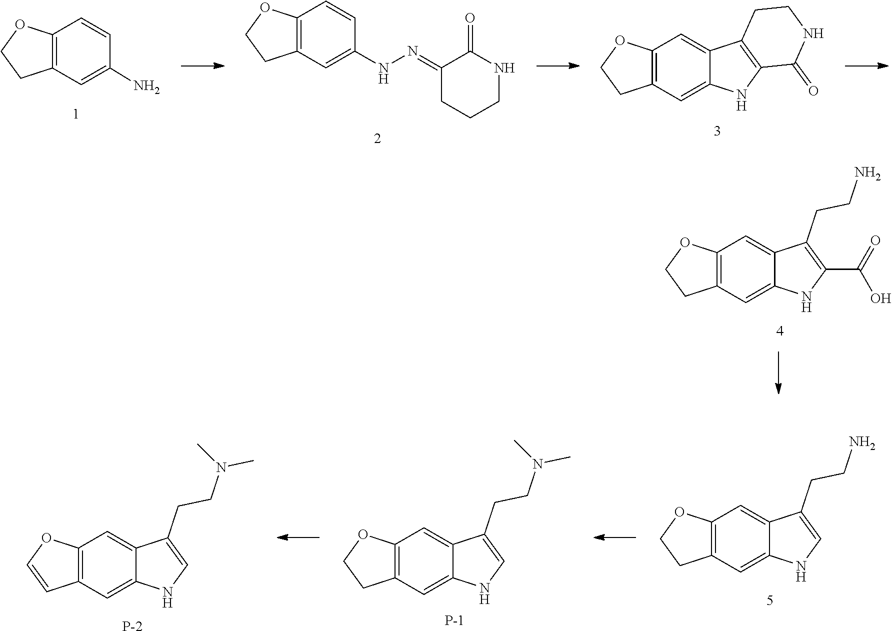

### Step 1: (E)-3-(2-(2,3-dihydrobenzofuran-5-yl)hydrazineylidene)piperidin-2-one (2)

A solution of ethyl 2-oxopiperidine-3-carboxylate (12.7 g, 74 mmol) in H₂O (120 mL) was treated with KOH (4.15 g, 74 mmol) and stirred at ambient temperature for 16 h. In a separate flask, a solution of 2,3-dihydro-1-benzofuran-5-amine (10.0 g, 74 mmol) in H₂O (120 mL) and 12 M aq. HCl (21.5 mL) was cooled to 0° C. and treated dropwise with a solution of NaNO₂ (5.1 g, 74 mmol) in H₂O (10 mL). This mixture was stirred at 0° C. for 30 min before being adjusted to pH 4 with 10% w/v aq. Na₂CO₃ and then added to the first solution containing the oxopiperidine. The pH of the combined reaction mixture was adjusted to pH 4 with AcOH and stirred at 0° C. for 16 h. Upon completion, the insoluble material was collected by filtration and washed with water (200 mL). The solid was dried under vacuum to afford a crude (E)-3-(2-(2,3-dihydrobenzofuran-5-yl)hydrazineylidene)piperidin-2-one (10 g) as a red solid which was used without further purification.

### Step 2: 2,3,5,7,8,9-hexahydro-6H-furo[2,3-f]pyrido[3,4-b]indol-6-one (3)

A solution of crude (E)-3-(2-(2,3-dihydrobenzofuran-5-yl)hydrazineylidene)piperidin-2-one (20.0 g) in HCOOH (120 mL) was stirred at 105° C. for 1 hr. The reaction mixture was diluted with H₂O (200 mL) and extracted with EtOAc (200 mL×2). The combined organic layers were washed with brine (100 mL), dried over Na₂SO₄, filtered and concentrated under reduced pressure to give crude 2,3,5,7,8,9-hexahydro-6H-furo[2,3-f]pyrido[3,4-b]indol-6-one (8.00 g) as a brown solid which was used in the subsequent step without further purification.

### Step 3: 7-(2-aminoethyl)-3,5-dihydro-2H-furo[2,3-f]indole-6-carboxylic acid (4)

To a solution of crude 2,3,5,7,8,9-hexahydro-6H-furo[2,3-f]pyrido[3,4-b]indol-6-one (7.50 g) in EtOH (20 mL) was added KOH (18.4 g, 328 mmol) and H₂O (20 mL). The mixture was stirred at 90° C. for 12 h. The solvent was removed, the residue was diluted with H₂O (25 mL) and then filtered. The filtrate was acidified with AcOH to produce a precipitate which was collected, washed with H₂O, and dried to give crude 7-(2-aminoethyl)-3,5-dihydro-2H-furo[2,3-f]indole-6-carboxylic acid (5.00 g) as a brown solid which was used in the subsequent step without further purification.

### Step 4: 2-(3,5-dihydro-2H-furo[2,3-f]indol-7-yl)ethan-1-amine (5)

To a solution of crude 7-(2-aminoethyl)-3,5-dihydro-2H-furo[2,3-f]indole-6-carboxylic acid (5.00 g, 20.3 mmol) in HCl (12 M aq., 10 mL) was added H₂O (20 mL) and the resulting mixture was stirred at 100° C. for 1 h. The reaction mixture was adjusted to pH 10 with 30% aq. NaOH. The H₂O was removed by lyophilization to give crude 2-(3,5-Dihydro-2H-furo[2,3-f]indol-7-yl)ethan-1-amine (3.00 g) as a brown solid which was used in the subsequent step without further purification.

### Step 5: 2-(3,5-dihydro-2H-furo[2,3-f]indol-7-yl)-N,N-dimethylethan-1-amine (P-1)

To a solution of crude 2-(3,5-dihydro-2H-furo[2,3-f]indol-7-yl)ethan-1-amine (3.00 g) in MeOH (15 mL) was added AcOH (3.56 g, 59.3 mmol, 3.39 mL), NaBH₃CN (1.86 g, 29.6 mmol) and 37% w/w aqueous formaldehyde (3.01 g, 37.0 mmol). The mixture was stirred at 20° C. for 2 h. The reaction mixture was then concentrated under reduced pressure and the residue was adjusted to pH 8 with saturated aqueous NaHCO₃ and extracted with CH₂Cl₂ (50 mL×2). The combined organic layers were washed with brine (50 mL), dried over anhydrous Na₂SO₄, filtered and the filtrate concentrated in vacuo to give a residue that was purified by preparative HPLC (column: Phenomenex luna C18 (250×50 mm×10 μm; mobile phase: [water (TFA)-ACN]; B: 1-40%, 10 min) to afford 2-(3,5-dihydro-2H-furo[2,3-f]indol-7-yl)-N,N-dimethylethan-1-amine as the trifluoroacetate salt (0.75 g, 3% over 5 steps) which was a yellow oil. ¹H NMR (400 MHZ, DMSO-d₆): δ 10.70 (s, 1H), 9.42 (br s, 1H), 7.19 (s, 1H), 7.11 (d, J=2.4 Hz, 1H), 6.89 (s, 1H), 4.47 (d, J=8.4 Hz, 2H), 3.28-3.25 (m, 2H), 3.22-3.18 (m, 2H), 2.99-2.95 (m, 2H), 2.84 (s, 6H). LCMS (ESI+): m/z=231.0 [M+H]⁺. HPLC Purity (220 nm): 98.2%.

### Step 6: 2-(5H-Furo[2,3-f]indol-7-yl)-N,N-dimethylethan-1-amine (P-2)

To a solution of 2-(3,5-dihydro-2H-furo[2,3-f]indol-7-yl)-N,N-dimethylethan-1-amine (0.50 g, 2.17 mmol) in dioxane (3 mL) was added DDQ (591 mg, 2.61 mmol). The mixture was stirred at 105° C. for 2 h. The reaction mixture was then concentrated under reduced pressure and the residue was purified by preparative HPLC (column: Waters Xbridge Prep OBD C18 150×40 mm×10 μm; mobile phase: [water (NH₃+NH₄HCO₃)-ACN]; B: 1-45%, 8 min) to give 2-(5H-furo[2,3-f]indol-7-yl)-N,N-dimethylethan-1-amine (68 mg, 14%) as an off-white solid. ¹H NMR (400 MHZ, CD₃CN-d₃): δ 8.93 (s, 1H), 7.65 (d, J=2.4 Hz, 1H), 7.61 (s, 1H), 7.52 (s, 1H), 7.14 (d, J=2.4 Hz, 1H), 6.84-6.83 (m, 1H), 2.91-2.87 (m, 2H), 2.60-2.56 (m, 2H), 2.25 (s, 6H). LCMS (ESI+): m/z=229.1 [M+H]⁺. HPLC Purity (220 nm): 99.7%.

Scheme 2: Compounds of general formula (I) can be synthesised from the appropriately substituted aniline following the outlined sequence of steps in Scheme 2 or similar as one skilled in the art may consider. Fischer indole synthesis utilising the appropriately substituted aniline allowed access to tetracyclic intermediate 292 that upon subsequent ring opening and hydrogenation generated intermediate 9. Decarboxylation generated intermediate 10. Reductive alkylation of the pendent amine provided access to compounds of general formula (I) (exemplified by P-3). One skilled in the art will recognise that utilising alternate aldehydes or ketones would allow access to compounds of general formula (I) disclosed herein.

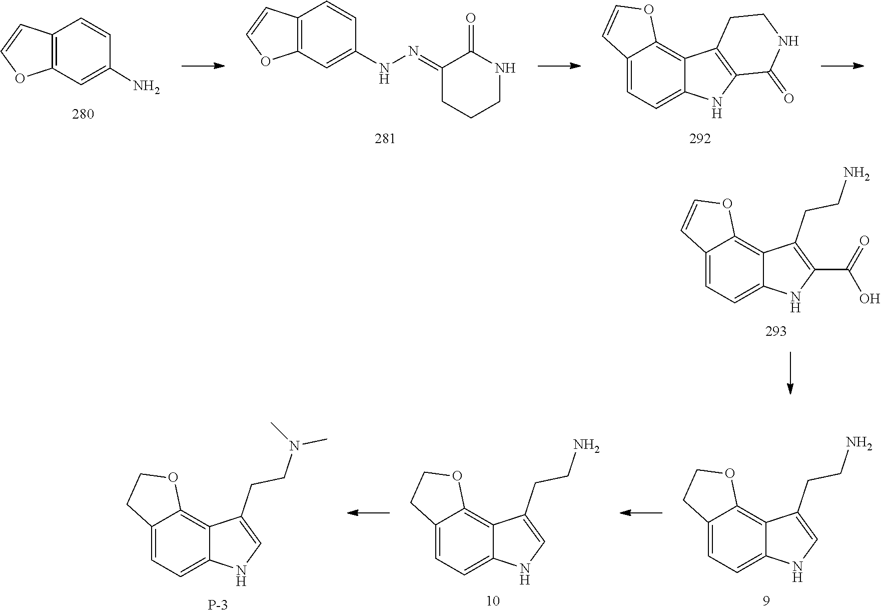

### Example 2: 2-(3,6-dihydro-2H-furo[2,3-e]indol-8-yl)-N,N-dimethylethan-1-amine (P-3)

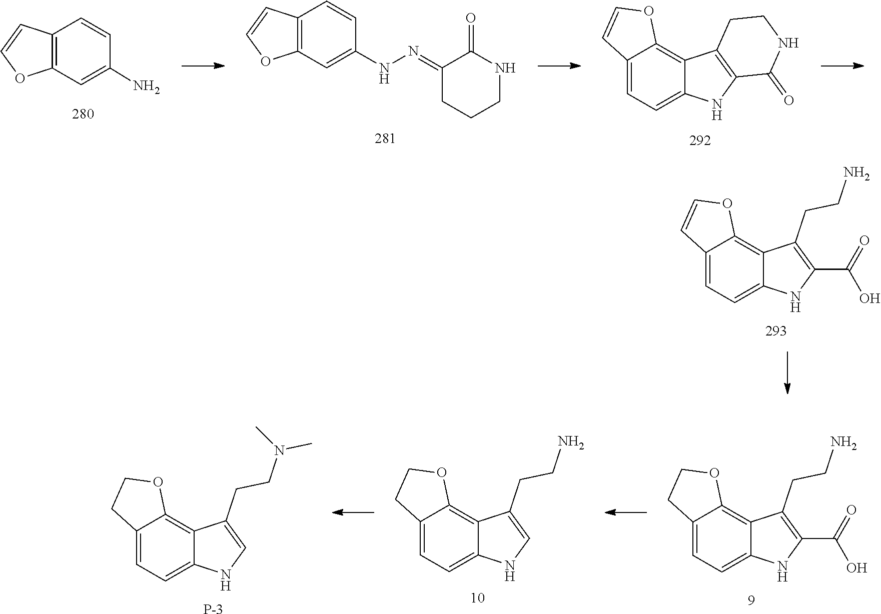

### Step 1: (E)-3-(2-(benzofuran-6-yl)hydrazineylidene)piperidin-2-one (281)

A solution of ethyl 2-oxopiperidine-3-carboxylate (6.43 g, 37.6 mmol) in H₂O (11 mL) was treated with KOH (2.11 g, 37.6 mmol) and stirred at ambient temperature for 16 h. In a separate flask, a solution of 1-benzofuran-6-amine (5 g, 37.6 mmol) in H₂O (11 mL) and 12 M aq. HCl (2.74 g, 2 eq., 75.1 mmol) was cooled to 0° C. and treated with NaNO₂ (2.59 g, 37.6 mmol), pre-dissolved in H₂O (11 mL). This mixture was stirred at 0° C. for 30 min before being adjusted to pH 4 with 10% w/v aq. Na₂CO₃ and then added to the first solution containing the oxopiperidine. The pH of the resulting mixture was adjusted to pH 5 with AcOH and stirred at 0° C. for 16 h. Upon completion, the insoluble material was collected by filtration and washed with H₂O (30 mL). The solid was dried in vacuo to afford a crude (E)-3-(2-(benzofuran-6-yl)hydrazineylidene)piperidin-2-one (5 g) as a brown solid which was used without further purification.

### Step 2: 6,8,9,10-tetrahydro-7H-furo[2,3-e]pyrido[3,4-b]indol-7-one (292)

A solution of crude (E)-3-(2-(benzofuran-6-yl)hydrazineylidene)piperidin-2-one (5.0 g) in EtOH (17.5 mL) was treated with a solution of 3 M HCl in MeOH (17.5 mL) and stirred at 80° C. for 3 h. The pH of the reaction mixture was adjusted to 8 with saturated aqueous Na₂CO₃ and the organic solvents were removed under reduced pressure. The residue was diluted with H₂O (20 mL) and then extracted with CH₂Cl₂ (30 mL×2). The combined organic layers were washed with brine (5 mL), dried over Na₂SO₄, filtered and the filtrate concentrated under reduced pressure to give crude 6,8,9,10-tetrahydro-7H-furo[2,3-e]pyrido[3,4-b]indol-7-one (4.00 g) as a brown solid which was used in the subsequent step without further purification.

### Step 3: 8-(2-aminoethyl)-6H-furo[2,3-e]indole-7-carboxylic acid (293)

To a solution of crude 6,8,9,10-tetrahydro-7H-furo[2,3-e]pyrido[3,4-b]indol-7-one (4.0 g) in EtOH (14 mL) was added KOH (19.8 g, 353 mmol) and H₂O (14 mL). The mixture was stirred at 90° C. for 16 h. The solvent was removed, the residue was diluted with H₂O (25 mL) and extracted with MTBE (30 mL×2). The aqueous layer was then filtered, and the filtrate was acidified with AcOH to produce a precipitate which was collected and washed with H₂O and then dried to afford crude 8-(2-aminoethyl)-6H-furo[2,3-e]indole-7-carboxylic acid (4.00 g) as a brown solid which was used in the subsequent step without further purification.

### Step 4: 8-(2-aminoethyl)-3,6-dihydro-2H-furo[2,3-e]indole-7-carboxylic acid (9)

A solution of crude 8-(2-aminoethyl)-6H-furo[2,3-e]indole-7-carboxylic acid (200 mg) in MeOH (10 mL) was treated with 10% Pd/C (87.1 mg, 0.82 mmol). The resulting mixture was stirred at 30° C. for 36 hours under an atmosphere of H₂ at 50 PSI. The suspension was then filtered through a pad of celite and the filter cake was eluted with MeOH (5 mL×3). The combined filtrate was concentrated in vacuo to afford a crude 8-(2-aminoethyl)-3,6-dihydro-2H-furo[2,3-e]indole-7-carboxylic acid (200 mg) as a brown oil which was used in the subsequent step without further purification.

### Step 5: 2-(3,6-dihydro-2H-furo[2,3-e]indol-8-yl)ethan-1-amine (10)

To a solution of crude 8-(2-aminoethyl)-3,6-dihydro-2H-furo[2,3-e]indole-7-carboxylic acid (200 mg) in H₂O (6 mL) was added HCl (12 M aq., 2 mL). The mixture was stirred at 100° C. for 1 h. The reaction mixture was adjusted to pH 13 with 10% aq. NaOH. The H₂O was then removed by lyophilization to give crude 2-(3,6-dihydro-2H-furo[2,3-e]indol-8-yl)ethan-1-amine (100 mg) as a brown solid which was used in the subsequent step without further purification.

### Step 6: 2-(3,6-dihydro-2H-furo[2,3-e]indol-8-yl)-N,N-dimethylethan-1-amine (P-3)

To a solution of crude 2-(3,6-dihydro-2H-furo[2,3-e]indol-8-yl)ethan-1-amine (100 mg) in MeOH (3 mL) was added AcOH (118 mg, 1.98 mmol), NaBH₃CN (62.1 mg, 0.99 mmol) and 37% w/w aqueous formaldehyde (100 mg, 1.24 mmol). The mixture was stirred at 20° C. for 12 h. The pH of the reaction mixture was then adjusted to 8 with saturated aqueous NaHCO₃ and then the methanol was removed under reduced pressure. The aqueous residue was extracted with CH₂Cl₂ (3 mL×2). The organic layers were washed with brine (1 mL×2), dried over anhydrous Na₂SO₄, filtered and the filtrate concentrated in vacuo. The residue was purified by preparative HPLC (column: Waters Xbridge Prep OBD C18 150×40 mm×10 μm; mobile phase: [water (NH₄HCO₃)-ACN]; B %: 1%-35%, 8 min) to give 2-(3,6-dihydro-2H-furo[2,3-e]indol-8-yl)-N,N-dimethylethan-1-amine (9.34 mg, 0.1% over 6 steps) as a yellow solid. 1H NMR (400 MHZ, CD₃CN-d₃): δ 8.89-8.93 (br. s, 1H), 6.97-6.92 (m, 2H), 6.85 (d, J=8.0 Hz, 1H), 4.63 (t, J=8.8 Hz, 2H), 3.22 (t, J=8.8 Hz, 2H), 2.90-2.85 (m, 2H), 2.56-2.52 (m, 2H), 2.21 (s, 6H). LCMS (ESI+) m/z 231.2 [M+H]⁺. HPLC Purity (220 nm): 99.4%

Scheme 3: Compounds of general formula (I) can be synthesised in a similar sequence of steps as outlined in Scheme 1 & 2. Performing a Fischer indole synthesis upon the appropriately decorated aniline allowed access to tetracyclic intermediate 282 that upon subsequent ring opening and decarboxylation allowed for the formation of intermediate 284. Reductive alkylation of the pendent amine provided access to compounds of general formula (I) (exemplified by P-4). One skilled in the art will recognise that utilising alternate aldehydes or ketones would allow access to compounds of general formula (I) disclosed herein.

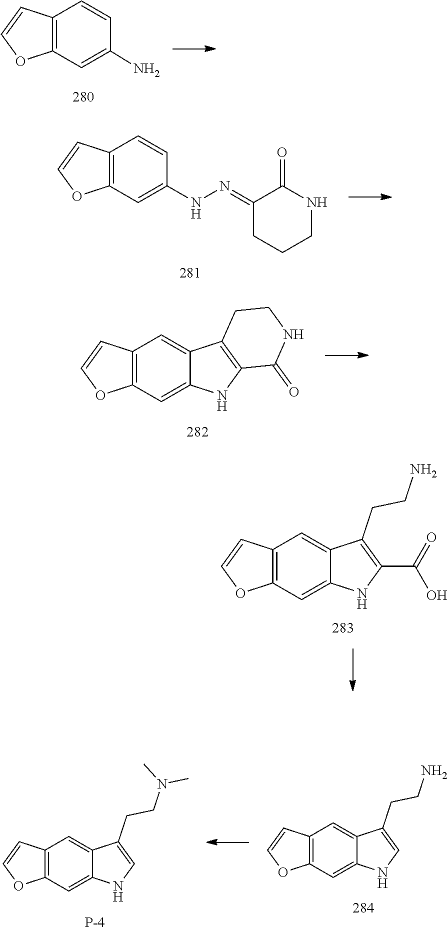

### Example 3: 2-(7H-furo[3,2-f]indol-5-yl)-N,N-dimethylethan-1-amine (P-4)

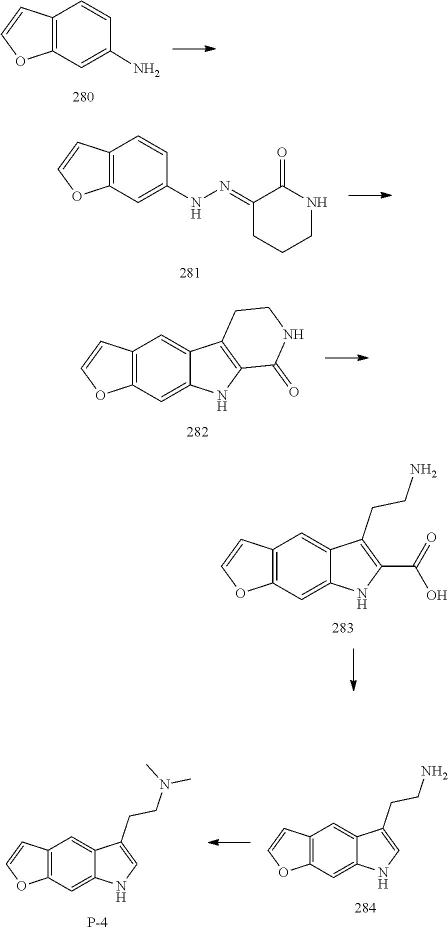

### Step 1: (E)-3-(2-(benzofuran-6-yl)hydrazineylidene)piperidin-2-one (281)

A solution of ethyl 2-oxopiperidine-3-carboxylate (6.43 g, 37.6 mmol) in H₂O (11 mL) was treated with KOH (2.11 g, 37.6 mmol) and stirred at ambient temperature for 16 h. In a separate flask, a solution of 1-benzofuran-6-amine (5 g, 37.6 mmol) in H₂O (11 mL) and 12 M aq. HCl (2.74 g, 2 eq., 75.1 mmol) was cooled to 0° C. and treated with NaNO₂ (2.59 g, 37.6 mmol), pre-dissolved in H₂O (11 mL). This mixture was stirred at 0° C. for 30 min before being adjusted to pH 4 with 10% w/v aq. Na₂CO₃ and then added to the first solution containing the oxopiperidine. The pH of the resulting mixture was adjusted to pH 5 with AcOH and stirred at 0° C. for 16 h. Upon completion, the insoluble material was collected by filtration and washed with H₂O (30 mL). The solid was dried in vacuo to afford a crude (E)-3-(2-(benzofuran-6-yl)hydrazineylidene)piperidin-2-one (5 g) as a brown solid which was used without further purification.

### Step 2: 5,6,7,9-tetrahydro-8H-furo[3,2-f]pyrido[3,4-b]indol-8-one (282)

A solution of crude (E)-3-(2-(benzofuran-6-yl)hydrazineylidene)piperidin-2-one (5.0 g, 20.5 mmol) in EtOH (17.5 mL) was treated with a solution of 3 M HCl in MeOH (17.5 mL) and was stirred at 80° C. for 3 h. The pH of the reaction mixture was adjusted to 8 with saturated aqueous Na₂CO₃ and the organic solvents were removed under reduced pressure. The residue was diluted with H₂O (20 mL) and then extracted with CH₂Cl₂ (30 mL×2). The combined organic layers were washed with brine (5 mL), dried over Na₂SO₄, filtered and the filtrate concentrated under reduced pressure to give crude 5,6,7,9-tetrahydro-8H-furo[3,2-f]pyrido[3,4-b]indol-8-one (4.00 g) as a brown solid which was used in the subsequent step without further purification.

### Step 3: 5-(2-aminoethyl)-7H-furo[3,2-f]indole-6-carboxylic acid (283)

To a solution of crude 5,6,7,9-tetrahydro-8H-furo[3,2-f]pyrido[3,4-b]indol-8-one (4.0 g) in EtOH (14 mL) was added KOH (19.8 g, 353 mmol) and H₂O (14 mL). The mixture was stirred at 90° C. for 16 h. The solvent was removed, the residue was diluted with H₂O (25 mL) and extracted with MTBE (30 mL×2). The aqueous layer was then filtered, and the filtrate was acidified with AcOH to produce a precipitate which was collected and washed with H₂O and then dried to afford crude 5-(2-aminoethyl)-7H-furo[3,2-f]indole-6-carboxylic acid (4.00 g) as a brown solid which was used in the subsequent step without further purification.

### Step 4: 2-(7H-furo[3,2-f]indol-5-yl)ethan-1-amine (284)

A solution of crude 5-(2-aminoethyl)-7H-furo[3,2-f]indole-6-carboxylic acid (4 g) in 12 M aqueous HCl (7 mL) and H₂O (21 mL) was stirred at 100° C. for 3.5 h. Upon completion, the pH of the reaction mixture was adjusted to 13 with 10% w/v aqueous NaOH and extracted with CH₂Cl₂ (50 mL×2). The combined organic layers were washed with brine (5 mL×2), dried over anhydrous Na₂SO₄, filtered, and the filtrate concentrated in vacuo to afford a crude 2-(7H-furo[3,2-f]indol-5-yl)ethan-1-amine (1 g) as a yellow solid which was used in the subsequent step without further purification.

### Step 5: 2-(7H-furo[3,2-f]indol-5-yl)-N,N-dimethylethan-1-amine (P-4)

A solution of crude 2-(7H-furo[3,2-f]indol-5-yl)ethan-1-amine (1 g) in MeOH (7 mL) was sequentially treated with acetic acid (1.2 g, 20 mmol), NaBH₃CN (628 mg, 9.99 mmol), and 37% w/w aqueous formaldehyde (1.01 g, 12.5 mmol) at 0° C. The resulting mixture was stirred at ambient temperature for 2 h. Upon completion, the pH of the reaction mixture was adjusted to 8 with saturated aqueous NaHCO₃ and the volatile components were removed in vacuo. The residue was diluted with H₂O (5 mL) and extracted with CH₂Cl₂ (10 mL×2). The combined organic layers were washed with brine (3 mL×2), dried over anhydrous Na₂SO₄, filtered, and the filtrate concentrated in vacuo. The residue was purified by preparative HPLC (column: Waters Xbridge Prep OBD C18 150×40 mm×10 μm; mobile phase: [water (aq. NH₃+NH₄HCO₃)-ACN]; B: 5-45%, 8 min) to afford 2-(7H-furo[3,2-f]indol-5-yl)-N,N-dimethylethan-1-amine (4.9 mg, 0.1% over 5 steps) as an off-white solid. ¹H NMR (400 MHZ, CD₃CN-d₃): δ 7.70 (d, J=2.0 Hz, 1H), 7.35-7.29 (m, 2H), 7.10 (d, J=1.6 Hz, 1H), 6.90 (d, J=2.4 Hz, 1H), 3.11-3.07 (m, 2H), 2.69-2.65 (m, 2H), 2.28 (s, 6H). LCMS (ESI+): m/z 229.0 [M+H]⁺. HPLC purity (220 nm): 89.2%.

Scheme 4: Compounds of general formula (I) can be synthesised from the appropriately substituted nitrotoluene following the outlined sequence of steps in Scheme 4 or similar as one skilled in the art may consider. A modified version of the Leimgruber-Batcho indole synthesis via conversion of an appropriately substituted nitroarene to a substituted styrene followed by a reductive cyclisation allowed access to indole intermediates (for example 17). Glyoxylation of such indoles with oxalyl chloride followed by treatment with an appropriately substituted amine gave glyoxamide intermediates which when subjected to reductive conditions provided compounds of general formula (I) (exemplified by P-6). One skilled in the art will recognise that utilising differentially substituted amines would allow access to compounds of general formula (I) disclosed herein.

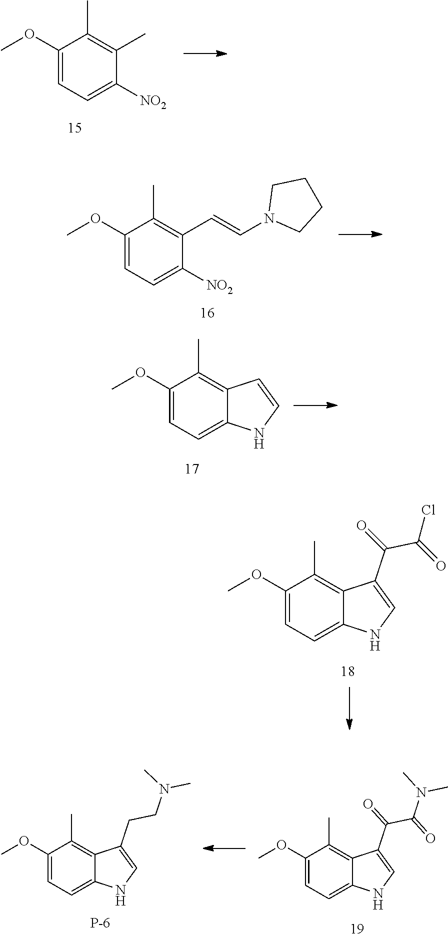

### Example 4: 2-(5-methoxy-4-methyl-1H-indol-3-yl)-N,N-dimethylethan-1-amine (P-6)

### Step 1: (E)-1-(3-methoxy-2-methyl-6-nitrostyryl)pyrrolidine (16)

To a solution of 1-methoxy-2,3-dimethyl-4-nitrobenzene (30.0 g, 166 mmol) in DMF (300 mL) was added DMF-DMA (39.4 g, 331 mmol) and pyrrolidine (14.1 g, 199 mmol). The reaction mixture was stirred at 130° C. for 12 h. The mixture was concentrated under reduced pressure to give crude (E)-1-(3-methoxy-2-methyl-6-nitrostyryl)pyrrolidine (39.1 g) as a black oil which was used in the subsequent step without further purification.

### Step 2: 5-methoxy-4-methyl-1H-indole (17)

To a solution of crude (E)-1-(3-methoxy-2-methyl-6-nitrostyryl)pyrrolidine (39.1 g) in AcOH (390 mL) was added Fe (92.4 g, 1.66 mol) in portions at 50° C. The reaction mixture was stirred at 80° C. for 4 h. The mixture was then filtered, and the filter cake was washed with EtOAc (80 mL). 6 N aqueous NaOH was added to the filtrate with stirring until the pH of the aqueous layer was 6-7. The layers were separated, and the aqueous layer was further extracted with EtOAc (400 mL×3). The combined organic layers were washed with brine (300 mL), dried over Na₂SO₄, filtered and the filtrate concentrated. The residue was purified by column chromatography (SiO₂, 1-5% EtOAc in petroleum ether) to give 5-methoxy-4-methyl-1H-indole (11.9 g, 45%) as a purple solid. ¹H NMR (400 MHZ, CDCl₃): δ 8.03 (s, 1H), 7.21-7.19 (m, 2H), 6.94 (d, J=8.8 Hz, 1H), 6.55-6.53 (m, 1H), 3.89 (s, 3H), 2.48 (s, 3H).

### Step 3: 2-(5-methoxy-4-methyl-1H-indol-3-yl)-2-oxoacetyl chloride (18)

A solution of oxalyl chloride (319 μL, 3.72 mmol) in anhydrous Et₂O (300 μL) was added dropwise to an ice-cold (0° C.) stirred solution of 5-methoxy-4-methyl-1H-indole (0.5 g, 3.1 mmol) in anhydrous Et₂O (20 mL) under N₂. The reaction was stirred at 0° C. for 30 min and the dark red precipitate was collected by filtration. The precipitate was washed with cold Et₂O (5 mL×2). A second crop of the title compound was obtained upon cooling the filtrate, precipitate filtered and added to the first batch to afford 2-(5-methoxy-4-methyl-1H-indol-3-yl)-2-oxoacetyl chloride (620 mg, 79%) as a red solid. ¹H NMR (400 MHZ, DMSO-d₆): δ 12.32 (s, 1H), 8.19 (d, J=3.6 Hz, 1H), 7.32 (d, J=8.8 Hz, 1H), 7.03 (d, J=8.8 Hz, 1H), 3.79 (s, 3H), 2.67 (s, 3H). ¹³C NMR (101 MHZ, DMSO-d₆): δ 181.4, 167.3, 153.4, 140.5, 133.0, 125.6, 118.8, 113.1, 110.3, 110.0, 56.7, 14.5.

### Step 4: 2-(5-methoxy-4-methyl-1H-indol-3-yl)-N,N-dimethyl-2-oxoacetamide (19)

To a solution of 2-(5-methoxy-4-methyl-1H-indol-3-yl)-2-oxoacetyl chloride (7.80 g, 27.8 mmol) dissolved in THF (50 mL) was added Me₂NH (2 M in THF, 35 mL) at 0° C. The reaction mixture was stirred at 25° C. for 12 h. The mixture was concentrated under reduced pressure and the residue was purified by column chromatography (SiO₂, 10-100% EtOAc in petroleum ether) to give 2-(5-methoxy-4-methyl-1H-indol-3-yl)-N,N-dimethyl-2-oxoacetamide (1.50 g, 21%) as a yellow solid. ¹H NMR (400 MHz, MeOD-d₄): δ 7.93 (s, 1H), 7.27 (d, J=8.8 Hz, 1H), 7.03 (d, J=8.8 Hz, 1H), 3.85 (s, 3H), 3.08 (s, 3H), 3.04 (s, 3H), 2.76 (s, 3H).

### Step 5: 2-(5-methoxy-4-methyl-1H-indol-3-yl)-N,N-dimethylethan-1-amine (P-6)

To an ice-cold stirred solution of 2-(5-methoxy-4-methyl-1H-indol-3-yl)-N,N-dimethyl-2-oxoacetamide (0.50 g, 1.92 mmol) in anhydrous THF (5 mL) under N₂ was added LiAlH₄ (875 mg, 23.0 mmol) in portions. The reaction mixture was then heated to reflux and stirring continued for 3 h. The mixture was cooled to 10° C. and quenched by portionwise addition of Na₂SO₄·10H₂O (3.00 g). The mixture was filtered, the filter cake was washed with THF (20 mL) and the filtrate was concentrated under reduced pressure. The crude product was purified by preparative HPLC (column: Waters Xbridge Prep OBD C18 (150×40 mm×10 μm); mobile phase: [water (NH₄HCO₃)-ACN]; B: 1-36%, 8 min) to give 2-(5-methoxy-4-methyl-1H-indol-3-yl)-N,N-dimethylethan-1-amine (110 mg, 24%) as an off-white solid. ¹H NMR (400 MHZ, MeOD-d₄): δ 7.10 (d, J=8.8 Hz, 1H), 7.00 (s, 1H), 6.84 (d, J=8.4 Hz, 1H), 3.80 (s, 3H), 3.11-3.07 (m, 2H), 2.68-2.64 (m, 2H), 2.55 (s, 3H), 2.38 (s, 6H). LCMS (ESI+): m/z 233.1 [M+H]⁺. HPLC purity (220 nm): 97.6%.

### Example 5: N-ethyl-2-(5-methoxy-4-methyl-1H-indol-3-yl)-N-methylethan-1-amine (P-37)

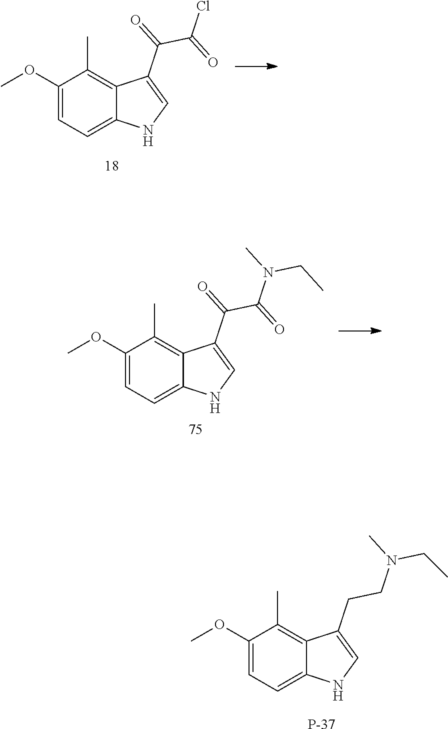

### Step 1: N-ethyl-2-(5-methoxy-4-methyl-1H-indol-3-yl)-N-methyl-2-oxoacetamide (75)

A suspension of 2-(5-methoxy-4-methyl-1H-indol-3-yl)-2-oxoacetyl chloride (360 mg, 1.43 mmol) in CH₂Cl₂ (10 mL) was treated with ethyl(methyl)amine (0.31 mL, 2.5 eq., 3.58 mmol) at 0° C. The reaction was stirred at ambient temperature for 30 min and then concentrated under a stream of N₂ gas. The oily residue was taken up in EtOAc (20 mL) and washed with 0.1 M aq. HCl (10 mL×3), H₂O (10 mL), and then brine (20 mL). The organic layer was dried over Na₂SO₄, filtered, and the filtrate concentrated in vacuo. The residue was purified by trituration with CH₂Cl₂/Et₂O to afford N-ethyl-2-(5-methoxy-4-methyl-1H-indol-3-yl)-N-methyl-2-oxoacetamide (380 mg, 97%) as a white solid which was a mixture of rotamers (A:B, 2:3). ¹H NMR (400 MHZ, MeOD-d₄): δ 7.92 (s, 0.6H, rotamer B), 7.88 (s, 0.4H, rotamer A), 7.27 (d, J=8.8 Hz, 1H), 7.04 (d, J=8.8 Hz, 1H), 3.85 (s, 3H), 3.56 (q, J=7.2 Hz, 0.8H, rotamer A), 3.39 (q, J=7.2 Hz, 1.2H, rotamer B), 3.06 (s, 1.8H, rotamer B), 3.01 (s, 1.2H, rotamer A), 2.76 (m, 3H), 1.26 (t, J=7.2 Hz, 1.2H, rotamer A), 1.19 (t, J=7.2 Hz, 1.8H, rotamer B).

### Step 2: N-ethyl-2-(5-methoxy-4-methyl-1H-indol-3-yl)-N-methylethan-1-amine (P-37)

To an ice-cold (0° C.) solution of anhydrous THF (10 mL) was added LiAlH₄ (221 mg, 8 eq., 5.83 mmol) in portions. The resulting ice-cold suspension was then treated dropwise with a solution of N-ethyl-2-(5-methoxy-4-methyl-1H-indol-3-yl)-N-methyl-2-oxoacetamide (0.2 g, 0.73 mmol) in anhydrous THF (2 mL). The reaction mixture was refluxed under N₂ for 3 h, cooled to 0° C., and quenched by sequential dropwise addition of H₂O (0.2 mL), 3.75 M aq. NaOH (0.2 mL), and H₂O (0.6 mL). The mixture was dried (Na₂SO₄) and filtered through a pad of celite. The filter cake was washed with additional hot THF (20 mL×2) and the combined filtrate was concentrated in vacuo to afford N-ethyl-2-(5-methoxy-4-methyl-1H-indol-3-yl)-N-methylethan-1-amine (160 mg, 89%) as a lightly coloured oil. ¹H NMR (400 MHZ, CDCl₃): δ 8.12 (s, 1H), 7.12 (d, J=8.8 Hz, 1H), 6.98 (d, J=1.8 Hz, 1H), 6.88 (d, J=8.8 Hz, 1H), 3.84 (s, 3H), 3.19-3.05 (m, 2H), 2.82-2.70 (m, 2H), 2.67-2.54 (m, 5H), 2.41 (s, 3H), 1.15 (t, J=7.2 Hz, 3H). ¹³C NMR (101 MHZ, CDCl₃): δ 151.4, 132.7, 126.9, 123.6, 118.3, 114.5, 110.2, 108.9, 59.0, 58.3, 51.5, 41.3, 24.9, 12.0, 11.8.

### Step 2a: N-ethyl-2-(5-methoxy-4-methyl-1H-indol-3-yl)-N-methylethan-1-amine fumarate (P-37⋅fumarate)

Fumaric acid (75 mg, 0.65 mmol) was dissolved in minimum refluxing acetone and treated with N-ethyl-2-(5-methoxy-4-methyl-1H-indol-3-yl)-N-methylethan-1-amine (160 mg, 0.65 mmol), pre-dissolved in minimal hot acetone. The resulting solution was allowed to cool to ambient temperature and stood overnight at 4° C. to afford the N-ethyl-2-(5-methoxy-4-methyl-1H-indol-3-yl)-N-methylethan-1-amine as the fumarate salt (127 mg, 60%) which were pale yellow crystals. ¹H NMR (400 MHZ, DMSO-d₆): δ 10.63 (s, 1H), 7.18-6.92 (m, 2H), 6.82 (d, J=8.8 Hz, 1H), 6.49 (s, 1H), 3.73 (s, 3H), 3.04 (dd, J=9.6, 6.8 Hz, 2H), 2.77 (dd, J=9.6, 6.8 Hz, 2H), 2.67 (q, J=7.2 Hz, 2H), 2.47 (s, 3H), 2.41 (s, 3H), 1.08 (t, J=7.2 Hz, 3H). ¹³C NMR (101 MHZ, DMSO-d₆): δ 167.6, 150.2, 134.9, 132.4, 126.3, 124.3, 116.3, 112.0, 109.0, 108.9, 58.1, 57.4, 50.4, 40.3, 23.7, 11.6, 11.1. LCMS (ESI+): m/z: 247.3 [M+H]⁺. qNMR Purity (ERETIC): 95.8%.

### Example 6: N-(2-(5-methoxy-4-methyl-1H-indol-3-yl)ethyl)-N-methylpropan-2-amine (P-38)

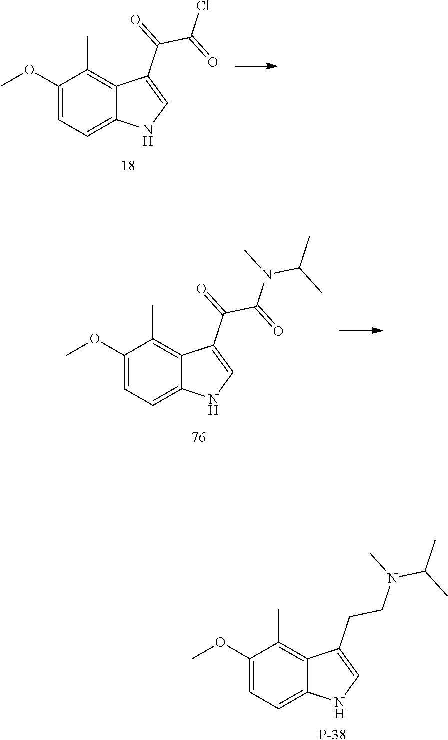

### Step 1: N-isopropyl-2-(5-methoxy-4-methyl-1H-indol-3-yl)-N-methyl-2-oxoacetamide (76)

To a solution of 2-(5-methoxy-4-methyl-1H-indol-3-yl)-2-oxoacetyl chloride (780 mg, 3.1 mmol) in THF (8 mL) was added N-methyl(propan-2-yl)amine (2.27 g, 31.0 mmol) in THF (5 mL) at 0° C. The reaction mixture was warmed to 25° C. and stirring continued for 12 h. The reaction mixture was then concentrated in vacuo and the residue was purified by column chromatography (SiO₂, 10% to 100% EtOAc in petroleum ether) to afford N-isopropyl-2-(5-methoxy-4-methyl-1H-indol-3-yl)-N-methyl-2-oxoacetamide (240 mg, 27%) as a yellow solid which was a mixture of rotamers. ¹H NMR (400 MHZ, MeOD-d₄): δ 7.92-7.85 (m, 1H), 7.28 (d, J=8.8 Hz, 1H), 7.04 (d, J=8.8 Hz, 1H), 3.86 (s, 3H), 3.49-3.43 (m, 1H), 2.77 (s, 3H), 2.01 (s, 3H), 1.21-1.22 (m, 6H). LCMS (ESI+): m/z 289.3 [M+H]⁺.

### Step 2: N-(2-(5-methoxy-4-methyl-1H-indol-3-yl)ethyl)-N-methylpropan-2-amine (P-38)

To an ice-cold (0° C.) solution of N-isopropyl-2-(5-methoxy-4-methyl-1H-indol-3-yl)-N-methyl-2-oxoacetamide (240 mg, 0.832 mmol) in THF (2.4 mL) was added LiAlH₄ (379 mg, 9.99 mmol) in portions under N₂. The reaction mixture was then warmed to 70° C. and stirring continued for 3 h. The reaction mixture was then cooled to 10° C., quenched by portionwise addition of Na₂SO₄·10H₂O (3 g), and filtered through a pad of celite. The filter cake was washed with THF (20 mL) and the combined filtrate was concentrated under reduced pressure. The crude residue was purified by preparative HPLC (column: Waters Xbridge BEH C18 100×30 mm×10 μm; mobile phase: [water (NH₄HCO₃)-ACN]; B: 5-40%, 8 min) to afford N-(2-(5-methoxy-4-methyl-1H-indol-3-yl)ethyl)-N-methylpropan-2-amine (28 mg, 13%) as a brown solid. ¹H NMR (400 MHZ, MeOD-d₄): δ 7.11 (d, J=8.4 Hz, 1H), 7.01 (s, 1H), 6.84 (d, J=8.8 Hz, 1H), 3.81 (s, 3H), 3.11-3.02 (m, 2H), 2.96 (sept, J=6.4 Hz, 1H), 2.75-2.66 (m, 2H), 2.55 (s, 3H), 2.36 (s, 3H), 1.09 (d, J=6.4 Hz, 6H). LCMS (ESI+) m/z 261.3 [M+H]⁺. HPLC Purity (220 nm): 96.1%

### Example 7: N-ethyl-N-(2-(5-methoxy-4-methyl-1H-indol-3-yl)ethyl)propan-2-amine (P-40)

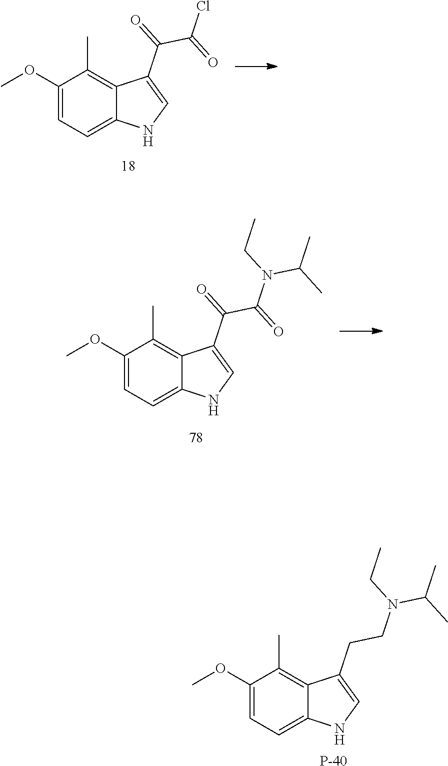

### Step 1: N-ethyl-N-isopropyl-2-(5-methoxy-4-methyl-1H-indol-3-yl)-2-oxoacetamide (78)

To an ice-cold solution of 2-(5-methoxy-4-methyl-1H-indol-3-yl)-2-oxoacetyl chloride (624 mg, 2.48 mmol) in THF (5 mL) was added N-ethylpropan-2-amine (2.16 g, 24.8 mmol) in THF (5 mL). The reaction mixture was warmed to 25° C. and stirring continued for 12 h at which point the volatiles were removed in vacuo and the resultant residue purified by column chromatography (SiO₂, 10-100% EtOAc in petroleum ether) to afford N-ethyl-N-isopropyl-2-(5-methoxy-4-methyl-1H-indol-3-yl)-2-oxoacetamide (541 mg) as a yellow solid which was used in the subsequent step without further purification.

### Step 2: N-ethyl-N-(2-(5-methoxy-4-methyl-1H-indol-3-yl)ethyl)propan-2-amine (P-40)

To an ice-cold stirred solution of crude N-ethyl-N-isopropyl-2-(5-methoxy-4-methyl-1H-indol-3-yl)-2-oxoacetamide (541 mg) in THF (7 mL) was added LiAlH₄ (813 mg, 21.4 mmol) in portions under N₂. The reaction mixture was then heated to 70° C. and stirring continued for 3 h. The reaction was then chilled to 10° C. and quenched by portionwise addition of Na₂SO₄·10H₂O (3 g) before being filtered through a pad of celite. The filter cake was washed with THF (20 mL) and combined filtrates concentrated under reduced pressure. The crude residue was purified by preparative HPLC (column: Waters Xbridge OBD C18 150×40 mm×10 μm; mobile phase: [water (NH₄HCO₃)-ACN]; B: 15-45%, 8 min) to afford N-ethyl-N-(2-(5-methoxy-4-methyl-1H-indol-3-yl)ethyl)propan-2-amine (67 mg, 10% over 2 steps) as an off-white solid. 1H NMR (400 MHZ, MeOD-d₄): δ 7.15 (d, J=8.8 Hz, 1H), 7.07 (s, 1H), 6.88 (d, J=8.8 Hz, 1H), 3.84 (s, 3H), 3.33-3.24 (m, 1H), 3.18-3.10 (m, 2H), 2.93-2.79 (m, 4H), 2.56 (s, 3H), 1.19 (t, J=7.2 Hz, 3H), 1.14 (d, J=6.8 Hz, 6H). LCMS: m/z: 275.1 [M+H]⁺. HPLC Purity (220 nm): 99.5%.

Scheme 5: Compounds of general formula (I) can be synthesised from the appropriately substituted nitrotoluene in a similar manner to those outlined in Scheme 5. A modified version of the Leimgruber-Batcho indole synthesis via conversion to an appropriately substituted styrene allowed access to the indole intermediates. Glyoxylation of such indoles with oxalyl chloride followed by treatment with an appropriately substituted amine gave glyoxamide intermediates which when subjected to reductive conditions provides access to compounds of general formula (I) (exemplified by P-7). One skilled in the art will recognise that utilising differentially substituted amines would allow access to compounds of general formula (I) disclosed herein.

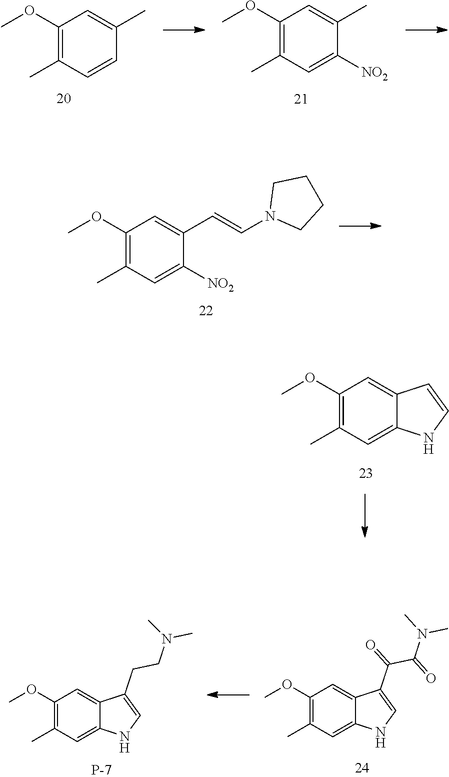

### Example 10: 2-(5-methoxy-6-methyl-1H-indol-3-yl)-N,N-dimethylethan-1-amine (P-7)

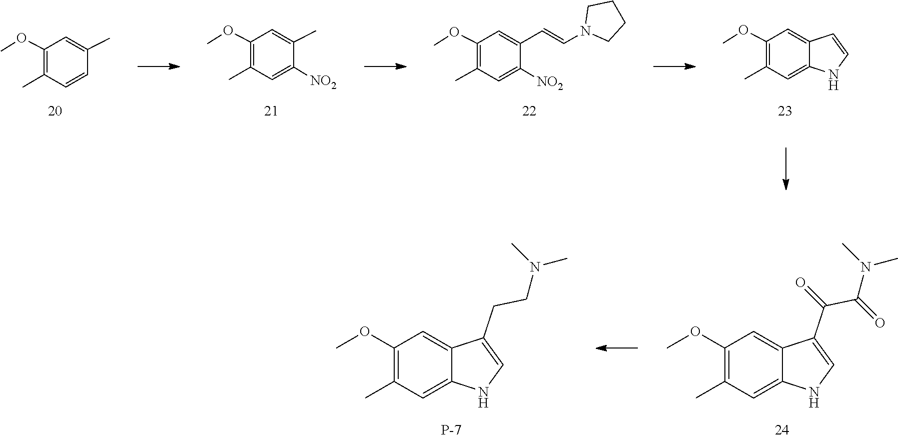

### Step 1: 1-methoxy-2,5-dimethyl-4-nitrobenzene (21)

2-Methoxy-1,4-dimethylbenzene (10.5 g, 76.7 mmol) was added dropwise to HNO₃ (70 mL) at 0-5° C., followed by NaNO₂ (15.9 g, 230 mmol) in portions at −5 to 2° C. The resulting mixture was stirred at 0-5° C. for 5 h. The mixture was quenched by pouring into ice H₂O (100 mL) and the pH was adjusted to 7-8 with 6 N aq. NaOH. The aqueous phase was extracted with EtOAc (60 mL×3). The organic layer was washed with brine (60 mL), dried over Na₂SO₄, filtered, and the filtrate concentrated. The residue was purified by column chromatography (SiO₂, 1-5% v/v EtOAc in petroleum ether) to afford 1-methoxy-2,5-dimethyl-4-nitrobenzene (6.5 g, 47%) as off-white solid. ¹H NMR (400 MHZ, CDCl₃): δ 7.93 (s, 1H), 6.66 (s, 1H), 3.91 (s, 3H), 2.64 (s, 3H), 2.22 (s, 3H).

### Step 2: (E)-1-(5-methoxy-4-methyl-2-nitrostyryl)pyrrolidine (22)

To a solution of 1-methoxy-2,5-dimethyl-4-nitrobenzene (7.40 g, 40.8 mmol) in DMF (50 mL) was added DMF-DMA (7.30 g, 61.3 mmol) and pyrrolidine (3.49 g, 49.0 mmol). The reaction mixture was stirred at 130° C. for 12 h. The reaction mixture was concentrated under reduced pressure to give crude (E)-1-(5-methoxy-4-methyl-2-nitrostyryl)pyrrolidine (9.60 g, crude) as black solid which was used in the subsequent step without further purification.

### Step 3: 5-methoxy-6-methyl-1H-indole (23)

To a solution of crude (E)-1-(5-methoxy-4-methyl-2-nitrostyryl)pyrrolidine (9.60 g) in AcOH (65 mL) was added Fe (22.7 g, 406 mmol) in portions at 50° C. The reaction mixture was stirred at 80° C. for 4 h. The mixture was filtered, and the filter cake was washed with EtOAc (20 mL). The combined filtrate was diluted with 6 N aq. NaOH until the pH was between 6-7. The layers were separated, and the aqueous phase was further extracted with EtOAc (100 mL×3). The combined organic layers were washed with brine (100 mL), dried over Na₂SO₄, filtered, and the filtrate concentrated. The residue was purified by column chromatography (SiO₂, 1-1.25% v/v EtOAc in petroleum ether) to afford 5-methoxy-6-methyl-1H-indole (1.66 g, 25%) as an off-white solid. ¹H NMR (400 MHZ, CDCl₃): δ 7.94 (s, 1H), 7.17 (s, 1H), 7.12 (t, J=2.8 Hz, 1H), 7.05 (t, J=2.0 Hz, 1H), 7.05 (s, 1H), 3.88 (s, 3H), 2.34 (s, 3H).

### Step 4: 2-(5-methoxy-6-methyl-1H-indol-3-yl)-N,N-dimethyl-2-oxoacetamide (24)

To a solution of 5-methoxy-6-methyl-1H-indole (2.43 g, 15.1 mmol) in THF (17 mL) was added (COCl)₂ (2.87 g, 22.6 mmol) dropwise at 0° C. under N₂. The reaction mixture was stirred at 0° C. for 2 h then a solution of Me₂NH (2 M in THF, 26 mL) was added dropwise. The reaction mixture was then stirred at 25° C. for 12 h. The mixture was concentrated under reduced pressure and the residue was purified by column chromatography (SiO₂, 1-100% v/v EtOAc in petroleum ether) to afford 2-(5-methoxy-6-methyl-1H-indol-3-yl)-N,N-dimethyl-2-oxoacetamide (2.30 g, 59%) as an off-white solid. ¹H NMR (400 MHZ, MeOD-d₄): δ 7.85 (s, 1H), 7.66 (s, 1H), 7.25 (s, 1H), 3.90 (s, 3H), 3.10 (s, 3H), 3.04 (s, 3H), 2.29 (s, 3H).

### Step 5: 2-(5-methoxy-6-methyl-1H-indol-3-yl)-N,N-dimethylethan-1-amine (P-7)

To a solution of 2-(5-methoxy-6-methyl-1H-indol-3-yl)-N,N-dimethyl-2-oxoacetamide (0.50 g, 1.92 mmol) in anhydrous THF (5 mL) was added LiAlH₄ (875 mg, 23.1 mmol) in portions under N₂. The mixture was stirred at 70° C. for 3 h, cooled to 0° C., and quenched with Na₂SO₄·10H₂O (3 g). The mixture was filtered, and the filter cake was washed with THF (20 mL). The combined filtrate was concentrated and the residue was purified by preparative HPLC (column: Waters Xbridge BEH C18 (100×30 mm×10 μm; mobile phase: [water (NH₄HCO₃)-ACN]; B: 20-40%, 8 min) to afford 2-(5-methoxy-6-methyl-1H-indol-3-yl)-N,N-dimethylethan-1-amine (101 mg, 23%) as an off-white solid. ¹H NMR: (400 MHZ, MeOD-d₄): δ 7.08 (s, 1H), 6.96 (s, 1H), 6.93 (s, 1H), 3.85 (s, 3H), 2.93-2.89 (m, 2H), 2.70-2.67 (m, 2H), 2.38 (s, 6H), 2.26 (s, 3H). LCMS (ESI+): m/z 233.2 [M+H]⁺. HPLC Purity (220 nm): 97.1%.

### Example 11: N-ethyl-2-(5-methoxy-6-methyl-1H-indol-3-yl)-N-methylethan-1-amine (P-42)

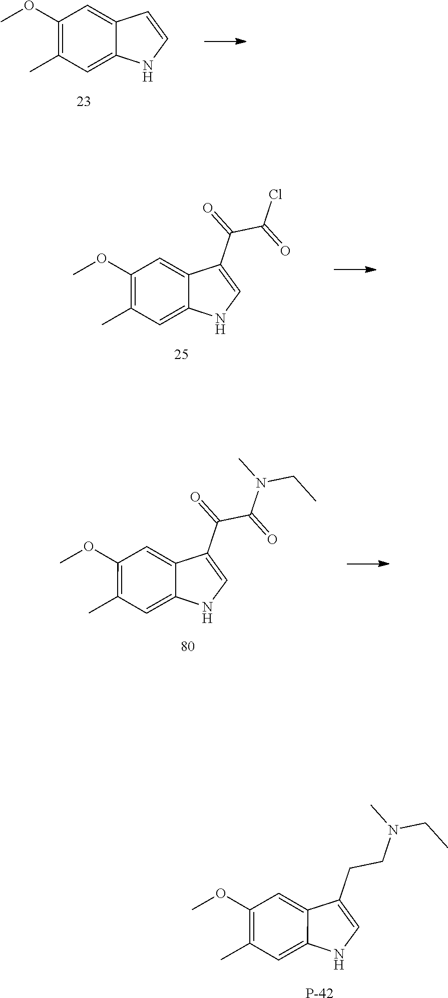

### Step 1: 2-(5-methoxy-6-methyl-1H-indol-3-yl)-2-oxoacetyl chloride (25)

To an ice-cold solution of 5-methoxy-6-methyl-1H-indole (2 g, 12.4 mmol) in anhydrous Et₂O (15 mL) was added a solution of (COCl)₂ (1.38 mL, 16.1 mmol) in anhydrous Et₂O (10 mL) dropwise. Stirring was continued for 2 h at which point the precipitate was collected by filtration and washed with anhydrous Et₂O (15 mL) to give 2-(5-methoxy-6-methyl-1H-indol-3-yl)-2-oxoacetyl chloride (2.6 g, 83%) as a red solid. ¹H NMR (400 MHZ, DMSO-d₆): δ 12.19 (br. s, 1H), 8.24 (d, J=3.2 Hz, 1H), 7.63 (s, 1H), 7.38-7.24 (m, 1H), 3.83 (s, 3H), 2.25 (s, 3H). ¹³C NMR (101 MHZ, DMSO-d₆): δ 180.6, 165.4, 154.5, 136.7, 131.0, 124.4, 123.3, 113.8, 112.3, 101.1, 55.3, 16.8.

### Step 2: N-ethyl-2-(5-methoxy-6-methyl-1H-indol-3-yl)-N-methyl-2-oxoacetamide (80)

To an ice-cold suspension of 2-(5-methoxy-6-methyl-1H-indol-3-yl)-2-oxoacetyl chloride (250 mg, 0.99 mmol) in CH₂Cl₂ (10 mL) was added ethyl(methyl)amine (0.26 mL, 2.98 mmol). Stirring was continued for 3 h at ambient temperature at which point HCl (0.1 M, 10 mL) was added. The aqueous phase was separated, extracted with CH₂Cl₂ (10 mL) and the combined organic layers were washed sequentially with H₂O (20 mL) and brine (20 mL) before being dried over MgSO₄, filtered, and the filtrate concentrated in vacuo. The residue was triturated with CH₂Cl₂/Et₂O (10 mL, v/v, 1/5) and the white precipitate filtered to give N-ethyl-2-(5-methoxy-6-methyl-1H-indol-3-yl)-N-methyl-2-oxoacetamide (248 mg, 91%) as a mixture of rotamers (A:B, approximately 1:2). ¹H NMR (400 MHZ, CDCl₃): δ 10.44 (m, 1H), 7.71 (s, 1H), 7.50-7.43 (m, 1H), 6.98 (s, 1H), 3.91 (s, 3H), 3.54 (q, J=7.2 Hz, 0.75H, rotamer A), 3.36 (q, J=7.2 Hz, 1.25H, rotamer B), 3.03-2.99 (m, 3H), 2.21 (s, 3H), 1.24-1.16 (m, 3H).

### Step 3: N-ethyl-2-(5-methoxy-6-methyl-1H-indol-3-yl)-N-methylethan-1-amine (P-42)

To an ice-cold solution of N-ethyl-2-(5-methoxy-6-methyl-1H-indol-3-yl)-N-methyl-2-oxoacetamide (250 mg, 0.91 mmol) in anhydrous THF (15 mL) was added LiAlH₄ (173 mg, 4.56 mmol) in portions. The resulting suspension was then heated at reflux for 1 h. The suspension was cooled in an ice bath and quenched by subsequent addition of H₂O (180 μL), 30% NaOH (w/v) (180 μL), H₂O (300 μL). The suspension was stirred at 0° C. for a further 1 h, Na₂SO₄ was added, and the reaction mass filtered through a pad of celite. The filtrate was concentrated under a stream of N₂ gas to give crude N-ethyl-2-(5-methoxy-6-methyl-1H-indol-3-yl)-N-methylethan-1-amine (0.2 g) as a colourless oil which was used in the subsequent step without further purification.

### Step 4a: N-ethyl-2-(5-methoxy-6-methyl-1H-indol-3-yl)-N-methylethan-1-amine (P-42⋅Fumarate)

A solution of N-ethyl-2-(5-methoxy-6-methyl-1H-indol-3-yl)-N-methylethan-1-amine (159 mg, 0.65 mmol) in hot acetone (2 mL) was added dropwise to a saturated solution of fumaric acid (74.9 mg, 0.65 mmol) in acetone at reflux. The colorless solution was allowed to cool to ambient temperature and stood at this temperature for 16 h. The resultant colourless needles were collected by vacuum filtration and dried in a vacuum desiccator to afford N-ethyl-2-(5-methoxy-6-methyl-1H-indol-3-yl)-N-methylethan-1-amine as the fumarate salt (80 mg, 36% over 2 steps) which were colourless crystals. ¹H NMR (400 MHZ, MeOD-d₄): δ 7.14 (s, 1H), 7.09 (s, 1H), 7.03 (s, 1H), 6.74 (s, 3H), 3.89 (s, 3H), 3.43 (t, J=7.6 Hz, 2H), 3.23 (m, 4H), 2.91 (s, 3H), 2.28 (s, 3H), 1.34 (t, J=7.2 Hz, 3H). LCMS (ESI+): m/z 247.1 [M+H]⁺. qNMR Purity (ERETIC): 99.9%.

### Step 4b: N-ethyl-2-(5-methoxy-6-methyl-1H-indol-3-yl)-N-methylethan-1-amine (P-42⋅Oxalate)

A solution of N-ethyl-2-(5-methoxy-6-methyl-1H-indol-3-yl)-N-methylethan-1-amine (246 mg, 1.0 mmol) in hot acetone (2 mL) was added dropwise to a solution of oxalic acid (100 mg, 1.1 mmol) dissolved in minimal acetone at reflux. The colorless solution was allowed to cool to ambient temperature and stood at this temperature for 16 h. The resultant colourless needles were collected by vacuum filtration and dried in a vacuum desiccator to afford N-ethyl-2-(5-methoxy-6-methyl-1H-indol-3-yl)-N-methylethan-1-amine oxalate (201 mg, 67%) as off-white crystals. ¹H NMR (400 MHZ, DMSO-d₆): δ 10.66 (s, 1H), 7.11 (s, 1H), 7.09 (d, J=2.4 Hz, 1H), 7.03 (s, 1H), 3.81 (s, 3H), 3.30-3.22 (m, 2H), 3.16 (q, J=7.2 Hz, 2H), 3.09-3.00 (m, 2H), 2.80 (s, 3H), 2.22 (s, 3H), 1.23 (t, J=7.2 Hz, 3H). ¹³C NMR (101 MHZ, DMSO-d₆): δ 164.6, 151.8, 130.9, 125.1, 122.4, 120.8, 112.7, 108.9, 98.5, 55.4, 54.6, 49.9, 38.5, 20.0, 16.9, 8.9. LCMS (ESI+): m/z 247.1 [M+H]⁺. qNMR Purity (ERETIC): 96.9%.

### Example 12: N-(2-(5-methoxy-6-methyl-1H-indol-3-yl)ethyl)-N-methylpropan-1-amine (P-43)

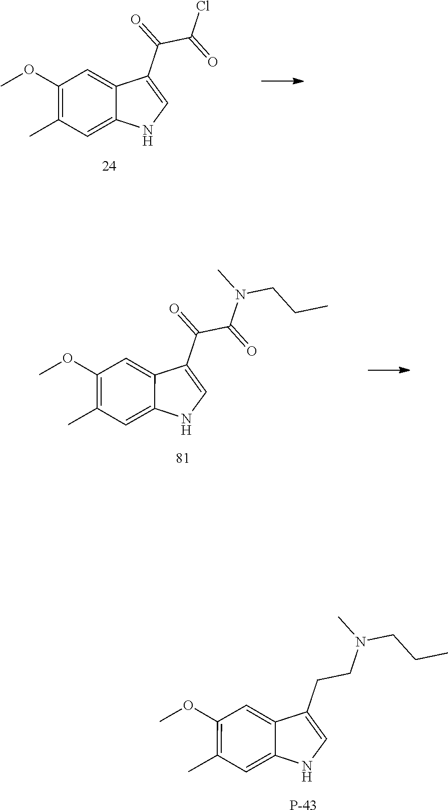

Step 1: 2-(5-methoxy-6-methyl-1H-indol-3-yl)-N-methyl-2-oxo-N-propylacetamide (81)

Methyl(propyl)amine (0.31 mL, 2.98 mmol) was added dropwise to an ice-cold suspension of 2-(5-methoxy-6-methyl-1H-indol-3-yl)-2-oxoacetyl chloride (250 mg, 0.99 mmol) in CH₂Cl₂ (10 mL). Stirring was continued for 3 h at which point aqueous HCl (0.1 M, 10 mL) was added. The aqueous phase was separated, extracted with CH₂Cl₂ (10 mL) and the combined organics washed sequentially with H₂O (20 mL) and brine (20 mL) before being dried over MgSO₄, filtered and the filtrate concentrated in vacuo yielding a yellow oil which was purified by flash column chromatography (SiO₂, 0.5-1% v/v. MeOH (10% aq. NH₃)—CH₂Cl₂) to give 2-(5-methoxy-6-methyl-1H-indol-3-yl)-N-methyl-2-oxo-N-propylacetamide (273 mg, 95%) as a mixture of rotamers (A:B). ¹H NMR (400 MHZ, CDCl₃): δ 9.73 (s, 0.5H, rotamer A), 9.68 (s, 0.5H, rotamer B), 7.73 (d, J=2.4 Hz, 1H), 7.62 (d, J=3.2 Hz, 0.5H, rotamer B), 7.56 (d, J=3.2 Hz, 0.5H, rotamer A), 7.09 (s, 1H), 3.93 (s, 3H), 3.57-3.44 (m, 1H, rotamer A), 3.34-3.26 (m, 1H, rotamer B), 3.04 (m, 3H), 2.27 (s, 3H), 1.77-1.56 (m, 2H), 1.01 (t, J=7.2 Hz, 1.5H, rotamer A), 0.82 (t, J=7.2 Hz, 1.5H, rotamer B).

### Step 2: N-(2-(5-methoxy-6-methyl-1H-indol-3-yl)ethyl)-N-methylpropan-1-amine (P-43)

To an ice-cold stirred solution of 2-(5-methoxy-6-methyl-1H-indol-3-yl)-N-methyl-2-oxo-N-propylacetamide (250 mg, 0.87 mmol) in anhydrous THF (15 mL) was added LiAlH₄ (165 mg, 4.34 mmol) in portions. The resulting suspension was then heated at reflux for 1 h. The suspension was cooled in an ice bath and quenched by subsequent addition of H₂O (180 μL), 30% NaOH (w/v) (180 μL), H₂O (300 μL). The suspension was stirred at 0° C. for a further 1 h, then Na₂SO₄ was added, and the reaction mass filtered through a pad of celite. The filter cake was washed with THF (20 mL×2) and the combined filtrates were concentrated under a stream of N₂ gas to give N-(2-(5-methoxy-6-methyl-1H-indol-3-yl)ethyl)-N-methylpropan-1-amine (180 mg, 80%) as a colourless oil.

### Example 13: N-(2-(5-methoxy-6-methyl-1H-indol-3-yl)ethyl)-N-methylpropan-2-amine (P-44)

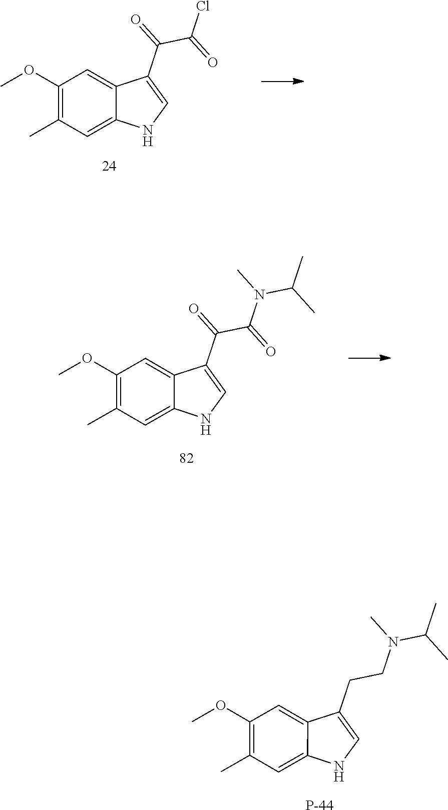

Step 1: N-isopropyl-2-(5-methoxy-6-methyl-1H-indol-3-yl)-N-methyl-2-oxoacetamide (82)

Methyl(propan-2-yl)amine (0.42 mL, 3.97 mmol) was added dropwise to an ice-cold suspension of 2-(5-methoxy-6-methyl-1H-indol-3-yl)-2-oxoacetyl chloride (250 mg, 0.99 mmol) in CH₂Cl₂ (10 mL). Stirring was continued for 3 h at which point aq. HCl (0.1 M, 10 mL) was added. The aqueous phase was separated, extracted with CH₂Cl₂ (10 mL) and the combined organic layers were washed sequentially with H₂O (20 mL) and brine (20 mL) before being dried over MgSO₄, filtered, and the filtrate concentrated in vacuo yielding a yellow oil. The oil was triturated with CH₂Cl₂/Et₂O (10 mL of a 1/5 (v/v) solution) and the white precipitate filtered to give N-isopropyl-2-(5-methoxy-6-methyl-1H-indol-3-yl)-N-methyl-2-oxoacetamide (258 mg, 90%) as a white powder which was a mixture of rotamers (A:B). ¹H NMR (400 MHZ, CDCl₃): δ 9.65 (s, 0.7H, rotamer B), 9.49 (s, 0.3H, rotamer A), 7.73 (s, 1H), 7.65 (d, J=3.2 Hz, 0.3H, rotamer A), 7.55 (d, J=3.2 Hz, 0.7H, rotamer B), 7.10 (s, 1H), 4.93 (p, J=6.8 Hz, 0.3H, rotamer A), 4.08 (h, J=6.8 Hz, 0.7H, rotamer B), 3.93 (s, 3H), 2.94 (s, 2H, rotamer B), 2.89 (s, 1H, rotamer A), 2.28 (s, 3H), 1.22 (m, 6H).

### Step 2: N-(2-(5-methoxy-6-methyl-1H-indol-3-yl)ethyl)-N-methylpropan-2-amine (P-44)

To an ice-cold stirred solution of N-isopropyl-2-(5-methoxy-6-methyl-1H-indol-3-yl)-N-methyl-2-oxoacetamide (250 mg, 0.87 mmol) in anhydrous THF (15 mL) was added LiAlH₄ (165 mg, 4.34 mmol) in portions. The resulting suspension was then heated at reflux for 1 h. The suspension was cooled in an ice bath and quenched by subsequent addition of H₂O (180 μL), 30% NaOH (w/v) (180 μL), H₂O (300 μL). The suspension was stirred at 0° C. for a further 1 h, then dried (Na₂SO₄), and the reaction mass filtered through a pad of celite. The residue was washed with THF (2×20 mL) and the combined filtrates were concentrated under a stream of N₂ gas to give N-(2-(5-methoxy-6-methyl-1H-indol-3-yl)ethyl)-N-methylpropan-2-amine (180 mg, 79%) as a colourless oil which was used in the subsequent step without further purification.

### Step 2a: N-(2-(5-methoxy-6-methyl-1H-indol-3-yl)ethyl)-N-methylpropan-2-amine fumarate (P-44⋅fumarate)

A solution of N-(2-(5-methoxy-6-methyl-1H-indol-3-yl)ethyl)-N-methylpropan-2-amine (168 mg, 0.65 mmol) in hot acetone (2 mL) was added dropwise to a saturated solution of fumaric acid (75 mg, 0.65 mmol) in acetone at reflux. The colorless solution was allowed to cool to ambient temperature and stood at this temperature for 16 h. The resultant colourless needles were collected by vacuum filtration and dried in a vacuum desiccator to afford N-(2-(5-methoxy-6-methyl-1H-indol-3-yl)ethyl)-N-methylpropan-2-amine as the fumarate salt (127 mg, 53%) which were colourless crystals. ¹H NMR (400 MHZ, MeOD-d₄): δ 7.14 (s, 1H), 7.10 (s, 1H), 7.03 (s, 1H), 6.73 (s, 2H), 3.88 (s, 3H), 3.67 (p, J=6.8 Hz, 1H), 3.37 (dd, J=9.6, 6.0 Hz, 2H), 3.23-3.15 (m, 2H), 2.83 (s, 3H), 2.28 (s, 3H), 1.32 (d, J=6.8 Hz, 6H). ¹³C NMR (101 MHZ, MeOD-d₄): δ 170.1, 152.6, 134.9, 131.7, 125.1, 122.2, 122.1, 112.5, 108.2, 97.8, 56.9, 54.9, 52.8, 34.6, 20.6, 15.8, 15.1. LCMS (ESI+): m/z 261.1 [M+H]⁺. qNMR Purity (ERETIC): 99.7%.

### Example 14: N, N-diethyl-2-(5-methoxy-6-methyl-1H-indol-3-yl)ethan-1-amine (P-45)

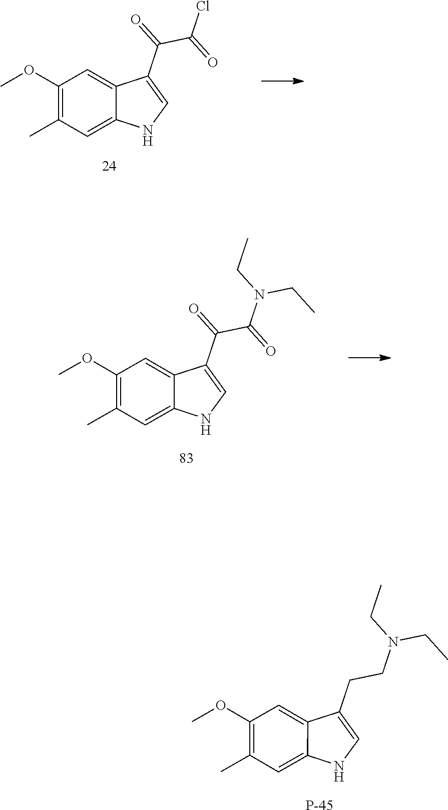

Step 1: N,N-diethyl-2-(5-methoxy-6-methyl-1H-indol-3-yl)-2-oxoacetamide (83)

A suspension of 2-(5-methoxy-6-methyl-1H-indol-3-yl)-2-oxoacetyl chloride (250 mg, 0.99 mmol) in anhydrous CH₂Cl₂ (10 mL) was added dropwise to an ice-cold stirred suspension of diethylamine (109 mg, 1.49 mmol) in CH₂Cl₂ (10 mL), followed by dropwise addition of triethylamine (0.46 mL, 3.28 mmol). Stirring was continued for 3 h at which point the reaction was diluted with H₂O (20 mL) and organic layer separated. The organic layer was washed with H₂O (2×25 mL) followed by brine (50 mL) before being dried over MgSO₄, filtered, and the filtrate concentrated under reduced pressure. The residue was triturated with CH₂Cl₂/Et₂O and then filtered to give N,N-diethyl-2-(5-methoxy-6-methyl-1H-indol-3-yl)-2-oxoacetamide (255 mg, 89%). ¹H NMR (400 MHZ, MeOD-d₄): δ 7.83 (s, 1H), 7.67 (s, 1H), 7.27 (d, J=1.2 Hz, 1H), 3.92 (s, 3H), 3.57 (q, J=7.2 Hz, 2H), 3.40 (q, J=7.2 Hz, 2H), 2.31 (s, 3H), 1.30 (t, J=7.2 Hz, 4H), 1.20 (t, J=7.2 Hz, 3H). ¹³C NMR (101 MHZ, MeOD-d₄): δ 186.6, 168.5, 155.3, 135.3, 131.7, 124.5, 124.0, 113.5, 113.1, 100.9, 54.6, 42.5, 38.8, 15.8, 13.0, 11.6.

### Step 2: N,N-diethyl-2-(5-methoxy-6-methyl-1H-indol-3-yl)ethan-1-amine (P-45)

To an ice-cold stirred solution of N, N-diethyl-2-(5-methoxy-6-methyl-1H-indol-3-yl)-2-oxoacetamide (250 mg, 0.87 mmol) in anhydrous THF (15 mL) was added LiAlH₄ (165 mg, 4.34 mmol) in portions. The resulting suspension was then heated at reflux for 1 h. The suspension was cooled in an ice bath and quenched by subsequent addition of H₂O (180 μL), 30% NaOH (w/v) (180 μL), H₂O (300 μL). The suspension was stirred at 0° C. for a further 1 h, then Na₂SO₄ was added, and the reaction mass filtered through a pad of celite and the residue washed with THF (2×20 mL). The combined filtrates were concentrated under a stream of N₂ gas to give N,N-diethyl-2-(5-methoxy-6-methyl-1H-indol-3-yl)ethan-1-amine (148 mg) as a colourless oil which was used in the subsequent step without further purification.

### Step 2a: N,N-diethyl-2-(5-methoxy-6-methyl-1H-indol-3-yl)ethan-1-amine (P-45-fumarate)

A solution of N,N-diethyl-2-(5-methoxy-6-methyl-1H-indol-3-yl)ethan-1-amine (140 mg, 0.54 mmol) in hot acetone (2 mL) was added dropwise to a saturated solution of fumaric acid (63 mg, 0.54 mmol) in acetone at reflux. The colorless solution was allowed to cool to ambient temperature and stood at this temperature for 16 h. The resultant colourless needles were collected by vacuum filtration and dried in a vacuum desiccator to afford N, N-diethyl-2-(5-methoxy-6-methyl-1H-indol-3-yl)ethan-1-amine as the fumarate salt (120 mg, 53% over 2 steps) which were colourless crystals. 1H NMR (400 MHZ, MeOD-d₄): δ 7.14 (d, J=1.2 Hz, 1H), 7.10 (s, 1H), 7.01 (s, 1H), 6.74 (s, 2H), 3.88 (s, 3H), 3.46-3.37 (m, 2H), 3.28 (m, 4H), 3.17 (m, 2H), 2.28 (s, 3H), 1.33 (t, J=7.2 Hz, 6H). ¹³C NMR (101 MHZ, MeOD-d₄): δ 168.9, 152.6, 134.5, 131.7, 125.0, 122.25, 122.24, 112.6, 108.2, 97.7, 54.8, 51.7, 46.9, 20.0, 15.8, 7.7. LCMS (ESI+): m/z 261.1 [M+H]⁺. qNMR Purity (ERETIC): 100%.

### Example 15: N-ethyl-N-(2-(5-methoxy-6-methyl-1H-indol-3-yl)ethyl)propan-1-amine (P-46)

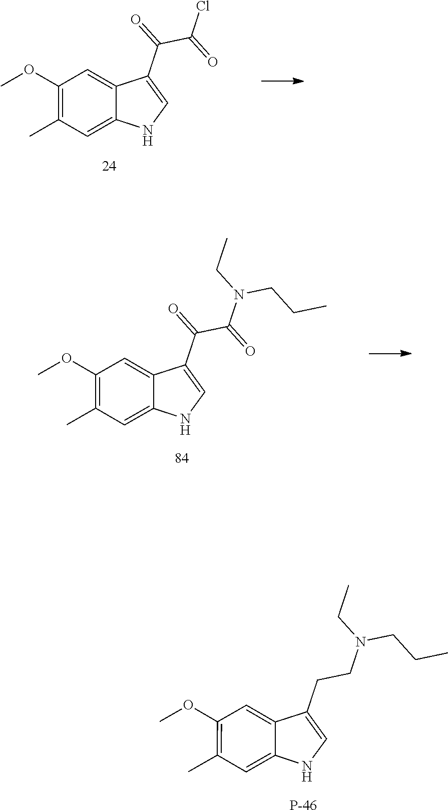

### Step 1: N-ethyl-2-(5-methoxy-6-methyl-1H-indol-3-yl)-2-oxo-N-propylacetamide (84)

Ethyl(propyl)amine (0.46 mL, 3.97 mmol) was added dropwise to an ice-cold suspension of 2-(5-methoxy-6-methyl-1H-indol-3-yl)-2-oxoacetyl chloride (250 mg, 0.99 mmol) in CH₂Cl₂ (10 mL). Stirring was continued for 3 h at which point 0.1 M aqueous HCl (10 mL) was added. The aqueous phase was separated, extracted with CH₂Cl₂ (10 mL) and the combined organic layers washed sequentially with H₂O (20 mL) and brine (20 mL) before being dried over MgSO₄, filtered, and the filtrate concentrated in vacuo yielding a yellow oil. The oil was triturated with CH₂Cl₂/Et₂O (10 mL of a 1/5 (v/v) solution) and the white precipitate collected by filtration to give N-ethyl-2-(5-methoxy-6-methyl-1H-indol-3-yl)-2-oxo-N-propylacetamide (278 mg, 93%) as a white powder as a mixture of rotamers (A:B). ¹H NMR (400 MHZ, CDCl₃): δ 10.02 (s, 1H), 7.72 (s, 1H), 7.51 (m, 1H), 7.05 (s, 1H), 3.93 (s, 3H), 3.55-3.52 (m, 1H), 3.49-3.32 (m, 2H), 3.31-3.22 (m, 1H), 2.25 (s, 3H), 1.78-1.55 (m, 2H), 1.28-1.16 (m, 3H), 1.01 (t, J=7.4 Hz, 1.6H, rotamer B), 0.81 (t, J=7.4 Hz, 1.4H, rotamer A).

### Step 2: N-ethyl-N-(2-(5-methoxy-6-methyl-1H-indol-3-yl)ethyl)propan-1-amine (P-46)

To an ice-cold stirred solution of N-ethyl-2-(5-methoxy-6-methyl-1H-indol-3-yl)-2-oxo-N-propylacetamide (262 mg, 0.87 mmol) in anhydrous THF (15 mL) was added LiAlH₄ (165 mg, 0.87 mmol) in portions. The resulting suspension was then heated at reflux for 1 h. The suspension was cooled in an ice bath and quenched by sequential addition of H₂O (180 μL), 30% NaOH (w/v) (180 μL), and H₂O (300 μL). The suspension was stirred at 0° C. for a further 1 h, then Na₂SO₄ was added, and the reaction mass filtered through a pad of celite. The residue was washed with THF (2×10 mL) and the combined filtrates were concentrated under a stream of N₂ gas to give N-ethyl-N-(2-(5-methoxy-6-methyl-1H-indol-3-yl)ethyl)propan-1-amine (185 mg, 78%) as a colourless oil which was used in the subsequent step without further purification.

### Step 2a: N-ethyl-N-(2-(5-methoxy-6-methyl-1H-indol-3-yl)ethyl)propan-1-amine fumarate (P-46-fumarate)

A solution of N-ethyl-N-(2-(5-methoxy-6-methyl-1H-indol-3-yl)ethyl)propan-1-amine (177 mg, 0.65 mmol) in hot acetone (2 mL) was added dropwise to a saturated solution of fumaric acid (75 mg, 0.65 mmol) in acetone at reflux. The colorless solution was allowed to cool to ambient temperature and stood at this temperature for 16 h. The resultant colourless needles were collected by vacuum filtration and dried in a vacuum desiccator to afford N-ethyl-N-(2-(5-methoxy-6-methyl-1H-indol-3-yl)ethyl)propan-1-amine as the fumarate salt (163 mg, 64%) which were colourless crystals. ¹H NMR (400 MHZ, MeOD-d₄): δ 7.13 (s, 1H), 7.07 (s, 1H), 7.01 (s, 1H), 6.73 (s, 1H), 3.87 (s, 3H), 3.38-3.30 (m, 2H), 3.24 (q, J=7.2 Hz, 2H), 3.18-3.04 (m, 4H), 2.28 (s, 3H), 1.80-1.66 (m, 2H), 1.31 (t, J=7.2 Hz, 3H), 0.99 (t, J=7.2 Hz, 3H). ¹³C NMR (101 MHz, MeOD-d₄): δ 173.1, 152.6, 135.9, 131.6, 125.1, 122.1, 122.0, 112.5, 108.7, 97.7, 54.9, 53.5, 52.1, 47.3, 20.0, 17.0, 15.9, 9.9, 7.7. LCMS (ESI+): m/z 275.2 [M+H]⁺. qNMR Purity (ERETIC): 98.7%.

### Example 16: N-ethyl-N-(2-(5-methoxy-6-methyl-1H-indol-3-yl)ethyl)propan-2-amine (P-47)

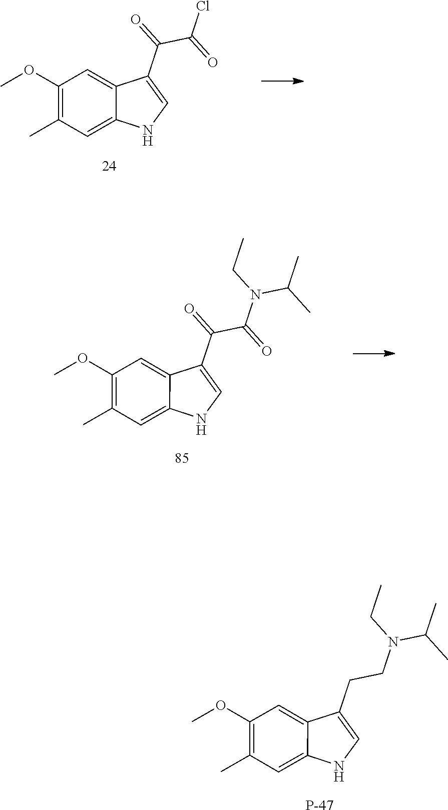

### Step 1: N-ethyl-N-isopropyl-2-(5-methoxy-6-methyl-1H-indol-3-yl)-2-oxoacetamide (85)

Ethyl(isopropyl)amine (463 μL, 3.97 mmol) was added dropwise to an ice-cold suspension of 2-(5-methoxy-6-methyl-1H-indol-3-yl)-2-oxoacetyl chloride (250 mg, 0.99 mmol) in CH₂Cl₂ (10 mL). Stirring was continued for 3 h at which point aqueous HCl (0.1 M, 10 mL) was added. The aqueous phase was separated, extracted with CH₂Cl₂ (10 mL) and the combined organics washed sequentially with H₂O (20 mL) and brine (20 mL) before being dried over anhydrous MgSO₄ and filtered and concentrated in vacuo. The oil was triturated with CH₂Cl₂/Et₂O (10 mL of a 1/5 (v/v) solution) and the white precipitate filtered to give N-ethyl-2-(5-methoxy-6-methyl-1H-indol-3-yl)-2-oxo-N-(propan-2-yl)acetamide (280 mg, 93%) as a white powder which was a mixture of rotamers (A:B). ¹H NMR (400 MHZ, CDCl₃): δ 9.69 (s, 0.7H, rotamer B), 9.54 (s, 0.3H, rotamer A), 7.73 (s, 1H), 7.62 (d, J=3.2 Hz, 0.3H, rotamer A), 7.55 (d, J=3.2 Hz, 0.7H, rotamer B), 7.12 (m, 1H), 4.62 (p, J=6.8 Hz, 0.3H, rotamer A), 4.11-4.00 (m, 0.7H, rotamer B), 3.93 (s, 3H), 3.39 (m, 2H), 2.29 (s, 3H), 1.39-1.16 (m, 9H).

### Step 2: N-ethyl-N-(2-(5-methoxy-6-methyl-1H-indol-3-yl)ethyl)propan-2-amine (P-47)

To an ice-cold stirred solution of N-ethyl-N-isopropyl-2-(5-methoxy-6-methyl-1H-indol-3-yl)-2-oxoacetamide (262 mg, 0.87 mmol) in anhydrous THF (15 mL) was added LiAlH₄ (165 mg, 4.34 mmol) in portions. The resulting suspension was then heated at reflux for 1 h. The suspension was cooled in an ice bath and quenched by subsequent addition of H₂O (180 μL), 30% NaOH (w/v) (180 μL), H₂O (300 μL). The suspension was stirred at 0° C. for a further 1 h, then Na₂SO₄ was added, and the reaction mass filtered through a pad of celite the filter cake was washed with THF (15 mL×2) and the combined filtrates concentrated under a stream of N₂ gas to give N-ethyl-N-(2-(5-methoxy-6-methyl-1H-indol-3-yl)ethyl)propan-2-amine (220 mg, 92%) as a colourless oil which was used in the subsequent step without further purification.

### Step 2a: N-ethyl-N-(2-(5-methoxy-6-methyl-1H-indol-3-yl)ethyl)propan-2-amine fumarate (P-47⋅fumarate)

A solution of N-ethyl-N-(2-(5-methoxy-6-methyl-1H-indol-3-yl)ethyl)propan-2-amine (177 mg, 0.65 mmol) in hot acetone (2 mL) was added dropwise to a saturated solution of fumaric acid (75 mg, 0.65 mmol) in acetone at reflux. The colorless solution was allowed to cool to ambient temperature and stood at this temperature for 16 h. The resultant colourless needles were collected by vacuum filtration and dried in a vacuum desiccator to afford N-ethyl-N-(2-(5-methoxy-6-methyl-1H-indol-3-yl)ethyl)propan-2-amine as the fumarate salt (220 mg, 87%) which were colourless crystals. ¹H NMR (400 MHZ, MeOD-d₄): δ 7.15 (s, 1H), 7.11 (s, 1H), 7.01 (s, 1H), 6.72 (s, 2H), 3.88 (s, 3H), 3.80 (sept, J=6.8 Hz, 1H), 3.42-3.37 (m, 2H), 3.29 (q, J=7.2 Hz, 2H), 3.24-3.16 (m, 2H), 2.28 (s, 3H), 1.42-1.33 (m, 9H). ¹³C NMR (101 MHZ, MeOD-d₄): δ 171.4, 154.0, 136.3, 133.1, 126.4, 123.63, 123.56, 114.0, 109.7, 99.0, 56.2, 56.0, 51.1, 46.7, 22.5, 17.2, 16.8, 10.6. LCMS (ESI+): m/z 275.2 [M+H]⁺. qNMR Purity (ERETIC): 99.6%.

### Example 17: N, N-dipropyl-2-(5-methoxy-6-methyl-1H-indol-3-yl)ethan-1-amine (P-48)

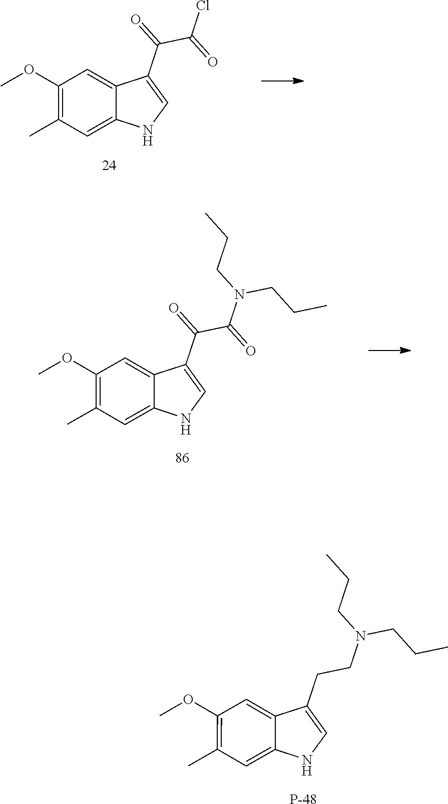

### Step 1: N,N-dipropyl-2-(5-methoxy-6-methyl-1H-indol-3-yl)-2-oxoacetamide (86)

A suspension of 2-(5-methoxy-6-methyl-1H-indol-3-yl)-2-oxoacetyl chloride (250 mg, 0.99 mmol) in anhydrous CH₂Cl₂ (10 mL) was added dropwise to an ice-cold stirred suspension of dipropylamine (151 mg, 1.49 mmol) in CH₂Cl₂ (10 mL) followed by dropwise addition of NEt₃ (457 μL, 3.28 mmol). Stirring was continued for 3 h at which point the reaction was diluted with H₂O (20 mL) and organic layer separated. The organic layer was washed with H₂O (2×25 mL) followed by brine (50 mL) before being dried over MgSO₄, filtered, and the filtrate concentrated under reduced pressure. The residue was triturated with CH₂Cl₂/Et₂₀ and filtered to give N,N-dipropyl-2-(5-methoxy-6-methyl-1H-indol-3-yl)-2-oxoacetamide (280 mg, 89%) as a white powder. ¹H NMR (400 MHZ, MeOD-d₄): δ 7.81 (s, 1H), 7.67 (s, 1H), 7.27 (d, J=1.2 Hz, 1H), 3.92 (s, 3H), 3.52-3.44 (m, 2H), 3.33-3.26 (m, 2H), 2.31 (s, 3H), 1.82-1.58 (m, 4H), 1.03 (t, J=7.6 Hz, 3H), 0.82 (t, J=7.6 Hz, 3H). ¹³C NMR (101 MHZ, MeOD-d₄): δ 186.5, 169.0, 155.3, 135.3, 131.6, 124.5, 124.0, 113.6, 113.1, 100.9, 54.6, 49.6, 45.9, 21.5, 20.2, 15.8, 10.3, 9.9.

### Step 2: N,N-dipropyl-2-(5-methoxy-6-methyl-1H-indol-3-yl)ethan-1-amine (P-48)

To an ice-cold stirred solution of 2-(5-methoxy-6-methyl-1H-indol-3-yl)-2-oxo-N,N-dipropylacetamide (250 mg, 0.79 mmol) in anhydrous THF (15 mL) was added LiAlH₄ (150 mg, 3.95 mmol) in portions. The resulting suspension was then heated at reflux for 1 h. The suspension was cooled in an ice bath and quenched by subsequent addition of H₂O (180 μL), 30% NaOH (w/v) (180 μL), H₂O (300 μL). The suspension was stirred at 0° C. for a further 1 h, then Na₂SO₄ was added, and the reaction mass filtered through a pad of celite. The residue was washed with THF (2×20 mL) and the combined filtrates were concentrated under a stream of N₂ gas to give N,N-dipropyl-2-(5-methoxy-6-methyl-1H-indol-3-yl)ethan-1-amine (200 mg, 88%) as a colourless oil which was used in the subsequent step without further purification.

### Step 2a: N, N-dipropyl-2-(5-methoxy-6-methyl-1H-indol-3-yl)ethan-1-amine fumarate (P-48⋅fumarate)

A solution of N,N-dipropyl-2-(5-methoxy-6-methyl-1H-indol-3-yl)ethan-1-amine (186 mg, 0.65 mmol) in hot acetone (2 mL) was added dropwise to a saturated solution of fumaric acid (75 mg, 0.65 mmol) in acetone at reflux. The colorless solution was allowed to cool to ambient temperature and stood at this temperature for 16 h. The resultant colourless needles were collected by vacuum filtration and dried in a vacuum desiccator to afford N, N-dipropyl-2-(5-methoxy-6-methyl-1H-indol-3-yl)ethan-1-amine as the fumarate salt (110 mg, 34% over 2 steps) which were colourless crystals. 1H NMR (400 MHZ, DMSO-d₆): δ 10.50 (s, 1H), 7.09 (s, 1H), 7.02 (d, J=2.4 Hz, 1H), 6.94 (s, 1H), 6.51 (s, 1H), 3.79 (s, 3H), 2.84 (s, 4H), 2.62 (t, J=7.6 Hz, 4H), 2.22 (s, 3H), 1.58-1.44 (m, 4H), 0.88 (t, J=7.2 Hz, 6H). ¹³C NMR (101 MHZ, DMSO-d₆): δ 167.7, 152.0, 135.2, 131.4, 126.0, 122.4, 120.9, 113.1, 112.0, 98.8, 55.7, 55.2, 54.1, 22.2, 19.5, 17.4, 12.1. LCMS (ESI+): m/z 289.2 [M+H]⁺. qNMR Purity (ERETIC): 99.8%.

### Example 18: N-isopropyl-N-(2-(5-methoxy-6-methyl-1H-indol-3-yl)ethyl)propan-2-amine (P-49)

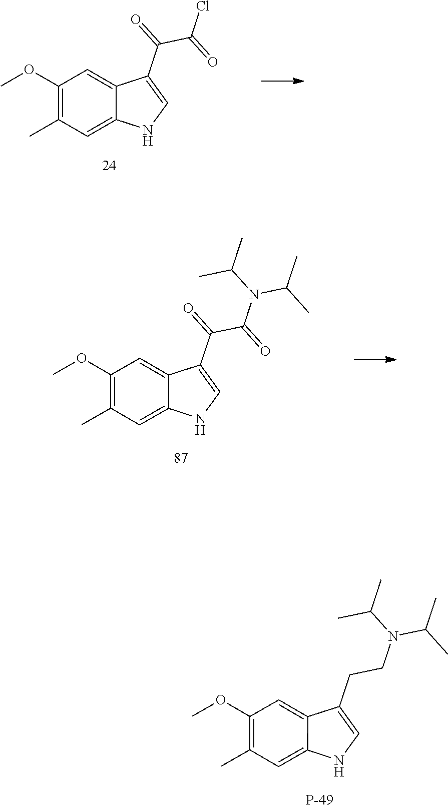

### Step 1: N,N-diisopropyl-2-(5-methoxy-6-methyl-1H-indol-3-yl)-2-oxoacetamide (87)

A solution of 2-(5-methoxy-6-methyl-1H-indol-3-yl)-2-oxoacetyl chloride (250 mg, 0.99 mmol) in anhydrous THF (5 mL) was cooled in an ice-bath and treated dropwise with diisopropylamine (418 μL, 2.98 mmol) under N₂. The reaction was stirred overnight at ambient temperature. The reaction was diluted with CH₂Cl₂ (20 mL) and filtered through a pad of celite under vacuum. The filtrate was concentrated in vacuo and the residue purified by flash chromatography (0% to 2% MeOH in CH₂Cl₂) to afford N,N-diisopropyl-2-(5-methoxy-6-methyl-1H-indol-3-yl)-2-oxoacetamide (150 mg, 48%). 1H NMR (400 MHZ, MeOD-d₄): δ 7.79 (s, 1H), 7.63 (s, 1H), 7.26 (s, 1H), 3.72-3.96 (m, 4H), 3.69 (p, J=6.8 Hz, 1H), 2.29 (s, 3H), 1.57 (d, J=6.8 Hz, 6H), 1.21 (d, J=6.8 Hz, 6H); ¹³C NMR (101 MHZ, MeOD-d₄): δ 188.2, 156.7, 149.3, 136.3, 125.9, 125.4, 116.0, 114.6, 110.7, 102.2, 56.1, 52.3, 47.2, 20.6, 20.6, 17.2; LRMS (ESI, −ve): m/z=315.4 [M−H]⁻.

### Step 2: N-isopropyl-N-(2-(5-methoxy-6-methyl-1H-indol-3-yl)ethyl)propan-2-amine (P-49)

To an ice-cold solution of N,N-diisopropyl-2-(5-methoxy-6-methyl-1H-indol-3-yl)-2-oxoacetamide (150 mg, 0.47 mmol) in anhydrous THF (10 mL) was added LiAlH₄ (144 mg, 3.79 mmol) in portions under N₂. The reaction was then stirred at RT for 16 h and then at reflux for 1 h. The suspension was cooled in an ice bath and quenched by subsequent addition of H₂O (150 μL), 30% NaOH (w/v) (150 μL), H₂O (300 μL). The suspension was stirred at 0° C. for a further 15 min, then MgSO₄ was added, and the reaction mass filtered through a pad of celite. The pad was further eluted with several volumes of EtOAc and the combined filtrate concentrated in vacuo to afford N-isopropyl-N-(2-(5-methoxy-6-methyl-1H-indol-3-yl)ethyl)propan-2-amine (90 mg, 66%) as a white solid. ¹H NMR (400 MHZ, MeOD-d₄): δ 7.09 (s, 1H), 6.97-6.89 (m, 2H), 3.84 (s, 3H), 3.29-3.17 (m, 2H), 2.93-2.76 (m, 4H), 2.26 (s, 3H), 1.18 (d, J=6.8 Hz, 12H). ¹³C NMR (101 MHZ, MeOD-d₄): δ 153.6, 133.0, 127.0, 122.9, 122.5, 113.7, 113.6, 99.5, 56.1, 51.8, 48.6, 28.3, 20.2, 17.2.

### Step 2a: N-isopropyl-N-(2-(5-methoxy-6-methyl-1H-indol-3-yl)ethyl)propan-2-amine hydrochloride (P-49⋅HCl)

A solution of N-isopropyl-N-(2-(5-methoxy-6-methyl-1H-indol-3-yl)ethyl)propan-2-amine in anhydrous Et₂O (1 mL) and anhydrous isopropanol (2 mL) was cooled to −10° C. and then 0.5M HCl in anhydrous Et₂O (2 mL) was added dropwise over 10 minutes. The solvent volume was then reduced with a stream of N₂ gas whilst still in the cold bath and a white precipitate slowly formed. The suspension was then diluted with anhydrous Et₂O (10 mL) at ambient temperature to promote precipitation. The precipitate was collected by filtration under a N₂ atmosphere and the solid residue was washed with Et₂O (1 mL) to afford N-isopropyl-N-(2-(5-methoxy-6-methyl-1H-indol-3-yl)ethyl)propan-2-amine as the hydrochloride salt (40 mg, 64%) which was a white solid. ¹H NMR (400 MHZ, D₂O): δ 7.26 (s, 1H), 7.15 (s, 1H), 7.06 (s, 1H), 3.82 (s, 3H), 3.70 (pent, J=6.8 Hz, 2H), 3.34-3.26 (m, 2H), 3.17-3.06 (m, 2H), 2.23 (s, 3H), 1.28 (d, J=6.8 Hz, 12H). ¹³C NMR (101 MHz, D₂O): δ 152.0, 131.5, 124.5, 123.7, 123.1, 113.4, 108.7, 99.7, 56.4, 55.1, 47.2, 22.9, 17.9, 16.0. LCMS (ESI+): m/z 289.2 [M+H]⁺. qNMR Purity (ERETIC): 99.4%.

Scheme 6: Compounds of general formula (I) can be synthesised from the appropriately substituted indole in a similar manner to those outlined in Scheme 6. Glyoxylation of appropriately substituted indoles with oxalyl chloride followed by treatment with an appropriately substituted amine gave glyoxamide intermediates which when subjected to reductive conditions provides access to compounds of general formula (I) (exemplified by P-50). One skilled in the art will recognise that utilising differentially substituted amines would allow access to compounds of general formula (I) disclosed herein.

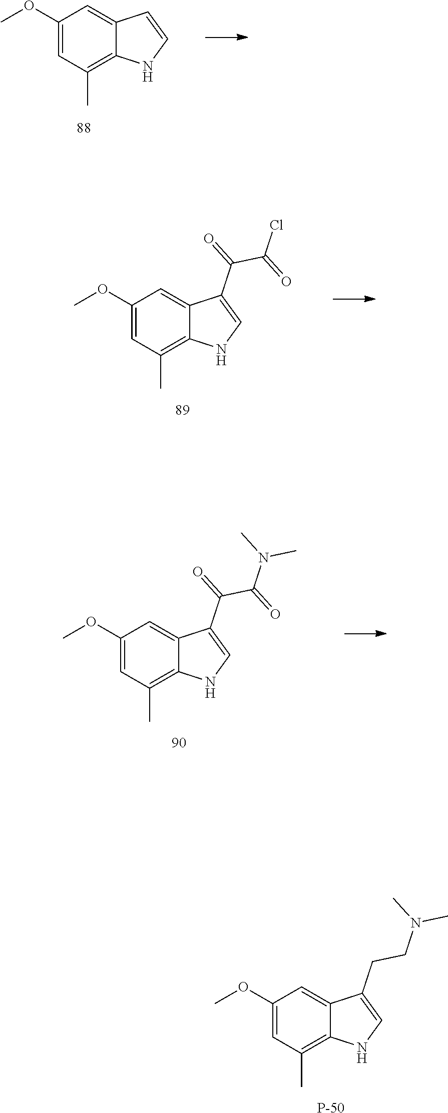

### Example 19: 2-(5-methoxy-7-methyl-1H-indol-3-yl)-N,N-dimethylethan-1-amine (P-50)

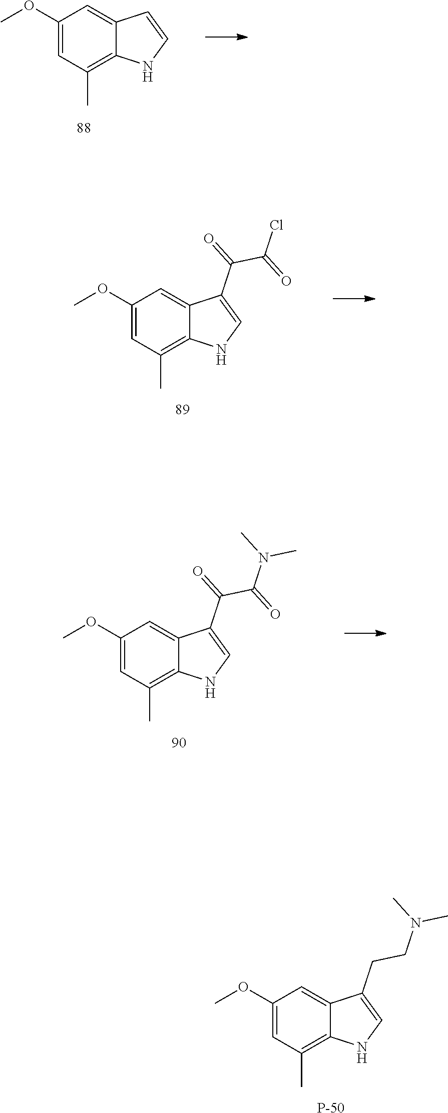

### Step 1: 2-(5-methoxy-7-methyl-1H-indol-3-yl)-2-oxoacetyl chloride (89)

A stirred solution of 5-methoxy-7-methyl-1H-indole (5.0 g, 31.0 mmol) in anhydrous Et₂O (35 mL) was cooled to 0° C. and treated with a solution of oxalyl chloride (3.46 mL, 40.3 mmol), dissolved in anhydrous Et₂O (35 mL) dropwise. Stirring was continued at this temperature for 1 h and then the red precipitate was collected by filtration and washed with cold anhydrous Et₂O (2×5 mL) to afford 2-(5-methoxy-7-methyl-1H-indol-3-yl)-2-oxoacetyl chloride (6.5 g, 83%) as a red powder. ¹H NMR (400 MHz, DMSO-d₆): δ 12.36 (s, 1H), 8.27 (d, J=3.2 Hz, 1H), 7.51 (d, J=2.4 Hz, 1H), 6.73 (d, J=2.4 Hz, 1H), 3.77 (s, 3H), 2.47 (s, 3H). ¹³C NMR (101 MHz, DMSO-d₆): δ 180.7, 165.4, 156.2, 137.3, 131.0, 126.2, 123.2, 113.9, 112.5, 100.5, 55.3, 16.7.

### Step 2: 2-(5-methoxy-7-methyl-1H-indol-3-yl)-N,N-dimethyl-2-oxoacetamide (90)

A suspension of 2-(5-methoxy-7-methyl-1H-indol-3-yl)-2-oxoacetyl chloride (0.4 g, 1.59 mmol) in anhydrous CH₂Cl₂ (10 mL) was added dropwise to an ice-cold stirred suspension of dimethylamine hydrochloride (389 mg, 4.77 mmol) in CH₂Cl₂ (10 mL) followed by dropwise addition of triethylamine (731 μL, 5.24 mmol). Stirring was continued for 3 h at which point the reaction was diluted with H₂O (20 mL) and organic layer separated. The organic layer was washed with H₂O (2×25 mL) followed by brine (50 mL) before being dried over MgSO₄, filtered, and the filtrate concentrated under reduced pressure. The residue was triturated with CH₂Cl₂/Et₂O, filtered to give 2-(5-methoxy-7-methyl-1H-indol-3-yl)-N,N-dimethyl-2-oxoacetamide (373 mg, 90%) as a white powder. ¹H NMR (400 MHZ, MeOD-d₄): δ 7.95 (s, 1H), 7.58 (s, 1H), 6.79 (s, 1H), 3.87 (s, 3H), 3.12 (s, 3H), 3.06 (s, 3H), 2.53 (s, 3H).

### Step 3: 2-(5-methoxy-7-methyl-1H-indol-3-yl)-N,N-dimethylethan-1-amine (P-50)

To an ice-cold stirred solution of 2-(5-methoxy-7-methyl-1H-indol-3-yl)-N,N-dimethyl-2-oxoacetamide (450 mg, 1.73 mmol) in anhydrous THF (15 mL) was added LiAlH₄ (197 mg, 5.19 mmol) in portions. The resulting suspension was then heated at reflux for 1 h. The suspension was cooled in an ice bath and quenched by subsequent addition of H₂O (100 μL), 30% aq. NaOH (w/v) (100 μL), H₂O (300 μL). The suspension was stirred at 0° C. for a further 1 h, then Na₂SO₄ was added, and the reaction mass filtered through a pad of celite. The filter cake was washed with THF (2×20 mL) and the combined filtrates were concentrated under a stream of N₂ gas to give 2-(5-methoxy-7-methyl-1H-indol-3-yl)-N,N-dimethylethan-1-amine (0.3 g) as a colourless oil which was used in the subsequent step without further purification.

### Step 3a: 2-(5-methoxy-7-methyl-1H-indol-3-yl)-N,N-dimethylethan-1-amine fumarate (P-50⋅fumarate)

A solution of 2-(5-methoxy-7-methyl-1H-indol-3-yl)-N,N-dimethylethan-1-amine (300 mg) in hot acetone (2 mL) was added dropwise to a saturated solution of fumaric acid (150 mg, 1.29 mmol) in acetone at reflux. The colorless solution was allowed to cool to ambient temperature and stood at this temperature for 16 h. The resultant colourless needles were collected by vacuum filtration and dried in a vacuum desiccator to afford 2-(5-methoxy-7-methyl-1H-indol-3-yl)-N,N-dimethylethan-1-amine as the fumarate salt (280 mg, 46% over 2 steps). ¹H NMR (400 MHZ, DMSO-d₆): δ 10.74 (d, J=2.8 Hz, 1H), 7.14 (d, J=2.4 Hz, 1H), 6.91 (d, J=2.4 Hz, 1H), 6.58-6.50 (m, 3H), 3.75 (s, 3H), 3.11-2.93 (m, 4H), 2.66 (s, 6H), 2.40 (s, 3H). ¹³C NMR (101 MHZ, DMSO-d₆): δ 168.4, 153.7, 135.5, 131.5, 127.2, 123.7, 122.0, 112.3, 110.8, 98.1, 57.7, 55.8, 43.0, 21.4, 17.2. LCMS (ESI+): m/z 233.2 [M+H]⁺. qNMR Purity (ERETIC): 98.9%.

### Example 20: N-ethyl-2-(5-methoxy-7-methyl-1H-indol-3-yl)-N-methylethan-1-amine (P-51)

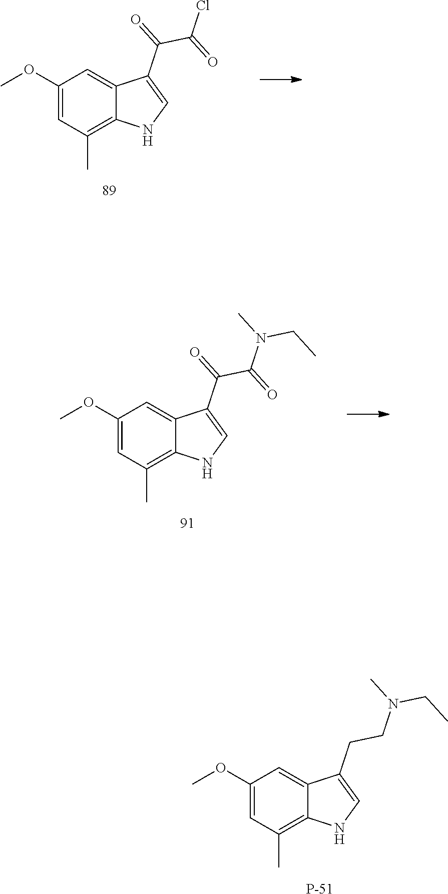

### Step 1: N-ethyl-2-(5-methoxy-7-methyl-1H-indol-3-yl)-N-methyl-2-oxoacetamide (91)

A solution of ethyl(methyl)amine (207 mg, 3.5 mmol) in CH₂Cl₂ (5 mL) was added dropwise to an ice-cold suspension of 2-(5-methoxy-7-methyl-1H-indol-3-yl)-2-oxoacetyl chloride (0.4 g, 1.59 mmol). Stirring was continued for 3 h at which point the reaction was diluted with H₂O (20 mL) and organic layer separated. The organic layer was washed subsequently with dilute HCl (0.1 M aq., 25 mL), H₂O (2×25 mL) followed by brine (50 mL) before being dried over MgSO₄, filtered, and the filtrate concentrated under reduced pressure. The residue was triturated with CH₂Cl₂/Et₂O and filtered to give N-ethyl-2-(5-methoxy-7-methyl-1H-indol-3-yl)-N-methyl-2-oxoacetamide (378 mg, 87%) as a white powder as a mixture of rotamers (A:B). ¹H NMR (400 MHZ, CDCl₃): δ 9.55 (s, 1H), 7.75-7.70 (m, 1H), 7.65-7.63 (m, 1H), 6.74 (s, 1H), 3.89 (s, 3H), 3.58 (q, J=7.2 Hz, 0.8H, rotamer A), 3.39 (q, J=7.2 Hz, 1.2H, rotamer B), 3.07 (s, 1.8H, rotamer B), 3.03 (s, 1.2H, rotamer A), 2.46 (s, 3H), 1.28-1.17 (m, 3H).

### Step 2: N-ethyl-2-(5-methoxy-7-methyl-1H-indol-3-yl)-N-methylethan-1-amine (P-51)

To an ice-cold stirred solution of N-ethyl-2-(5-methoxy-7-methyl-1H-indol-3-yl)-N-methyl-2-oxoacetamide (425 mg, 1.55 mmol) in anhydrous THF (15 mL) was added LiAlH₄ (176 mg, 3 eq., 4.65 mmol) in portions. The resulting suspension was then heated at reflux for 1 h. The suspension was cooled in an ice bath and quenched by subsequent addition of H₂O (180 μL), 30% NaOH (w/v) (180 μL), H₂O (360 μL). The suspension was stirred at 0° C. for a further 1 h, then Na₂SO₄ was added, and the reaction mass filtered through a pad of celite. The filtrate was concentrated under a stream of N₂ gas to give N-ethyl-2-(5-methoxy-7-methyl-1H-indol-3-yl)-N-methylethan-1-amine (0.3 g) as a colourless oil which was used in the subsequent step without further purification.

### Step 2a: N-ethyl-2-(5-methoxy-7-methyl-1H-indol-3-yl)-N-methylethan-1-amine fumarate (P-51⋅fumarate)

A solution of N-ethyl-2-(5-methoxy-7-methyl-1H-indol-3-yl)-N-methylethan-1-amine (159 mg, 0.65 mmol) in hot acetone (2 mL) was added dropwise to a saturated solution of fumaric acid (75 mg, 0.65 mmol) in acetone at reflux. The colorless solution was allowed to cool to ambient temperature and stood at this temperature for 16 h. The resultant colourless needles were collected by vacuum filtration and dried in a vacuum desiccator to afford N-ethyl-2-(5-methoxy-7-methyl-1H-indol-3-yl)-N-methylethan-1-amine as the fumarate salt (148 mg, 31% over 2 steps). ¹H NMR (400 MHZ, DMSO-d₆): δ 10.67 (s, 1H), 7.11 (d, J=2.4 Hz, 1H), 6.86 (d, J=2.4 Hz, 1H), 6.56-6.53 (m, 1H), 6.50 (s, 1H), 3.74 (s, 3H), 2.95-2.81 (m, 4H), 2.75 (q, J=7.2 Hz, 2H), 2.46 (s, 3H), 2.39 (s, 3H), 1.10 (t, J=7.2 Hz, 3H). ¹³C NMR (101 MHZ, DMSO-d₆): δ 168.2, 153.1, 135.2, 131.0, 126.9, 123.0, 121.4, 111.6, 111.5, 97.6, 56.0, 55.3, 50.0, 30.6, 21.5, 16.7, 10.6. LCMS (ESI+): m/z 247.1 [M+H]⁺. qNMR Purity (ERETIC): 99.0%.

### Example 21: N-(2-(5-methoxy-7-methyl-1H-indol-3-yl)ethyl)-N-methylpropan-1-amine (P-52)

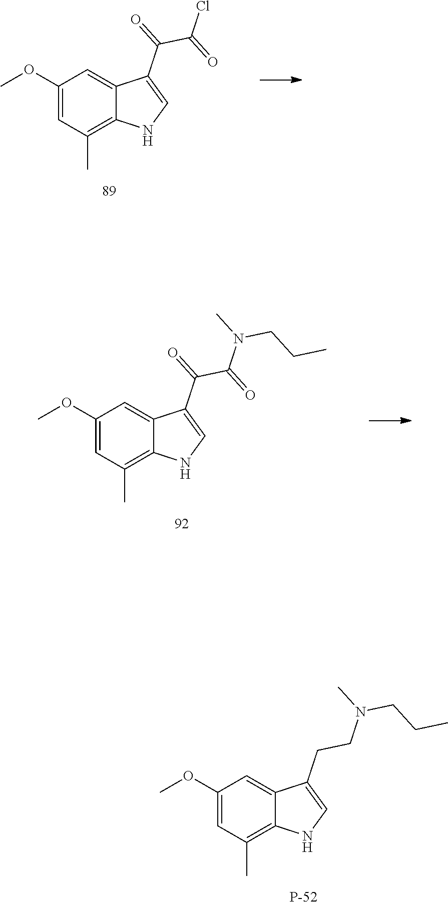

### Step 1: 2-(5-methoxy-7-methyl-1H-indol-3-yl)-N-methyl-2-oxo-N-propylacetamide (92)

A solution of methyl(propyl)amine (256 mg, 3.50 mmol) in CH₂Cl₂ (5 mL) was added dropwise to an ice-cold stirred suspension of 2-(5-methoxy-7-methyl-1H-indol-3-yl)-2-oxoacetyl chloride (0.4 g, 1.59 mmol). Stirring was continued for 3 h at which point the reaction was diluted with H₂O (20 mL) and organic layer separated. The organic layer was washed subsequently with dilute HCl (0.1 M aq., 25 mL), H₂O (2×25 mL) followed by brine (50 mL) before being dried over MgSO₄, filtered, and the filtrate concentrated under reduced pressure. The residue was triturated with CH₂Cl₂/Et₂O and filtered to give 2-(5-methoxy-7-methyl-1H-indol-3-yl)-N-methyl-2-oxo-N-propylacetamide (320 mg, 70%) as a white powder as a mixture of rotamers (A:B). ¹H NMR (400 MHZ, MeOD-d₄): δ 7.90-7.86 (m, 1H), 7.57 (d, J=2.4 Hz, 1H), 6.76 (d, J=2.4, 1H), 3.85 (s, 3H), 3.56-3.48 (m, 1H), 3.34-3.27 (m, 1H), 3.35-3.30 (m, 1H), 3.09 (s, 1.8H, rotamer B), 3.03 (s, 1.2H, rotamer A), 2.50 (s, 3H), 1.81-1.59 (m, 2H), 1.03 (t, J=7.2 Hz, 1.2H, rotamer A), 0.83 (t, J=7.2 Hz, 1.8H, rotamer B).

### Step 2: N-(2-(5-methoxy-7-methyl-1H-indol-3-yl)ethyl)-N-methylpropan-1-amine (P-52)

To an ice-cold stirred solution of 2-(5-methoxy-7-methyl-1H-indol-3-yl)-N-methyl-2-oxo-N-propylacetamide (395 mg, 1.37 mmol) in anhydrous THF (15 mL) was added LiAlH₄ (156 mg, 4.11 mmol) in portions. The resulting suspension was then heated at reflux for 1 h. The suspension was cooled in an ice bath and quenched by subsequent addition of H₂O (100 μL), 30% NaOH (w/v) (100 μL), H₂O (300 μL). The suspension was stirred at 0° C. for a further 1 h, then Na₂SO₄ was added, and the reaction mass filtered through a pad of celite, and the filter cake washed with THF (2×20 mL). The filtrate was concentrated under a stream of N₂ gas to give N-(2-(5-methoxy-7-methyl-1H-indol-3-yl)ethyl)-N-methylpropan-1-amine (295 mg) as a colourless oil which was used in the subsequent step without further purification.

### Step 2a: N-(2-(5-methoxy-7-methyl-1H-indol-3-yl)ethyl)-N-methylpropan-1-amine (P-52⋅fumarate)

A solution of N-(2-(5-methoxy-7-methyl-1H-indol-3-yl)ethyl)-N-methylpropan-1-amine (168 mg, 0.65 mmol) in hot acetone (2 mL) was added dropwise to a saturated solution of fumaric acid (74.9 mg, 0.65 mmol) in acetone at reflux. The colorless solution was allowed to cool to ambient temperature and held at this temperature overnight. The resultant colourless needles were collected by vacuum filtration and dried in a vacuum desiccator overnight yielding N-(2-(5-methoxy-7-methyl-1H-indol-3-yl)ethyl)-N-methylpropan-1-amine as the fumarate salt (219 mg, 42% over 2 steps). ¹H NMR (400 MHz, DMSO-d₆): δ 10.66 (s, 1H), 7.11 (d, J=2.4 Hz, 1H), 6.85 (d, J=2.4 Hz, 1H), 6.55-6.52 (m, 1H), 6.51 (s, 1H), 3.74 (s, 3H), 2.98-2.78 (m, 4H), 2.65-2.58 (m, 2H), 2.46 (s, 3H), 2.39 (s, 3H), 1.55 (sept, J=7.4 Hz, 2H), 0.88 (t, J=7.4 Hz, 3H). ¹³C NMR (101 MHZ, DMSO-d₆): δ 168.0, 153.1, 135.1, 131.0, 126.9, 123.0, 121.4, 111.6, 111.6, 97.6, 57.7, 56.7, 55.3, 40.5, 21.5, 18.6, 16.7, 11.5. LCMS (ESI+): m/z 261.1 [M+H]⁺. qNMR Purity (ERETIC): 97.9%.

### Example 22: N-(2-(5-methoxy-7-methyl-1H-indol-3-yl)ethyl)-N-methylpropan-2-amine (P-53)

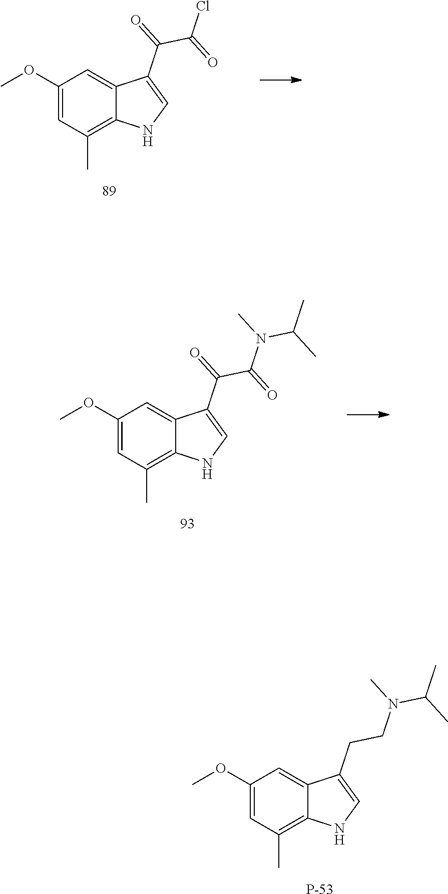

### Step 1: N-isopropyl-2-(5-methoxy-7-methyl-1H-indol-3-yl)-N-methyl-2-oxoacetamide (93)

A solution of methyl(propan-2-yl)amine (256 mg, 3.5 mmol) in CH₂Cl₂ (5 mL) was added dropwise to an ice-cold suspension of 2-(5-methoxy-7-methyl-1H-indol-3-yl)-2-oxoacetyl chloride (0.4 g, 1.59 mmol). Stirring was continued for 3 h at which point the reaction was diluted with H₂O (20 mL) and organic layer separated. The organic layer was washed subsequently with dilute HCl (0.1 M aq., 25 mL), H₂O (2×25 mL) followed by brine (50 mL) before being dried over MgSO₄, filtered, and the filtrate concentrated under reduced pressure. The residue was triturated with CH₂Cl₂/Et₂O and filtered to give N-isopropyl-2-(5-methoxy-7-methyl-1H-indol-3-yl)-N-methyl-2-oxoacetamide (401 mg, 88%) as a white powder which was a mixture of rotamers (A:B). ¹H NMR (400 MHZ, MeOD-d₄): δ 7.95 (s, 0.7H, rotamer B), 7.90 (s, 0.3H, rotamer A), 7.57 (m, 1H), 6.79 (m, 1H), 4.8 (sept, J=6.8 Hz, 0.3H, rotamer A), 4.02 (sept, J=6.8 Hz, 0.7H, rotamer B), 3.87 (s, 3H), 2.98 (s, 2H, rotamer B), 2.90 (s, 1H, rotamer A), 2.52 (s, 3H), 1.29 (d, J=6.8 Hz, 2H, rotamer A), 1.23 (d, J=6.8 Hz, 4H, rotamer B).

### Step 2: N-(2-(5-methoxy-7-methyl-1H-indol-3-yl)ethyl)-N-methylpropan-2-amine (P-53)

To an ice-cold stirred solution of 2-(5-methoxy-7-methyl-1H-indol-3-yl)-N-methyl-2-oxo-N-(propan-2-yl)acetamide (475 mg, 1.65 mmol) in anhydrous THF (15 mL) was added LiAlH₄ (188 mg, 4.94 mmol) in portions. The resulting suspension was then heated at reflux for 1 h. The suspension was cooled in an ice bath and quenched by subsequent addition of H₂O (180 μL), 30% NaOH (w/v) (180 μL), H₂O (300 μL). The suspension was stirred at 0° C. for a further 1 h, then Na₂SO₄ was added, and the reaction mass filtered through a pad of celite. The filtrate was concentrated under a stream of N₂ gas to give N-(2-(5-methoxy-7-methyl-1H-indol-3-yl)ethyl)-N-methylpropan-2-amine (340 mg, 79%) as a colourless oil which was used in the subsequent step without further purification.

### Step 2a: N-(2-(5-methoxy-7-methyl-1H-indol-3-yl)ethyl)-N-methylpropan-2-amine oxalate (P-53⋅oxalate)

A solution of N-(2-(5-methoxy-7-methyl-1H-indol-3-yl)ethyl)-N-methylpropan-2-amine (168 mg, 0.65 mmol) in hot acetone (2 mL) was added dropwise to a saturated solution of oxalic acid (75 mg, 0.65 mmol) in acetone at reflux. The colorless solution was allowed to cool to ambient temperature and stood at this temperature for 16 h. The resultant colourless needles were collected by vacuum filtration and dried in a vacuum desiccator to afford N-(2-(5-methoxy-7-methyl-1H-indol-3-yl)ethyl)-N-methylpropan-2-amine as the oxalate salt (180 mg, 31% over 2 steps). ¹H NMR (400 MHZ, DMSO-d₆): δ 10.78 (s, 1H), 7.19 (d, J=2.4 Hz, 1H), 6.90 (d, J=2.4 Hz, 1H), 6.61-6.42 (m, 1H), 3.75 (s, 3H), 3.60 (sept, J=6.4 Hz, 1H), 3.27-3.17 (m, 2H), 3.11-2.98 (m, 2H), 2.73 (s, 3H), 2.39 (s, 3H), 1.24 (d, J=6.4 Hz, 6H). qNMR Purity (ERETIC): 98.3%.

### Example 23: N, N-diethyl-2-(5-methoxy-7-methyl-1H-indol-3-yl)ethan-1-amine (P-54)

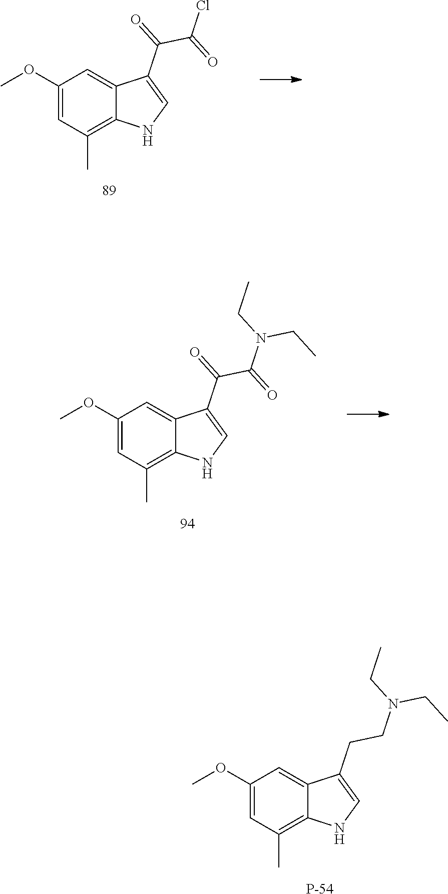

### Step 1: N,N-diethyl-2-(5-methoxy-7-methyl-1H-indol-3-yl)-2-oxoacetamide (94)

A solution of diethylamine (174 mg, 2.38 mmol) in CH₂Cl₂ (5 mL) was added dropwise to an ice-cold suspension 2-(5-methoxy-7-methyl-1H-indol-3-yl)-2-oxoacetyl chloride (0.4 g, 1.59 mmol) in CH₂Cl₂ (10 mL). Stirring was continued for 3 h at which point the reaction was diluted with H₂O (20 mL) and organic layer separated. The organic layer was washed subsequently with dilute HCl (0.1 M aq., 25 mL), H₂O (2×25 mL) followed by brine (50 mL) before being dried over MgSO₄, filtered, and the filtrate concentrated under reduced pressure. The residue was triturated with CH₂Cl₂/Et₂O and filtered to give N,N-diethyl-2-(5-methoxy-7-methyl-1H-indol-3-yl)-2-oxoacetamide (330 mg, 72%) as a white powder. ¹H NMR (400 MHZ, CDCl₃): δ 9.98 (s, 1H), 7.63-7.59 (m, 2H), 6.73-6.71 (m, 1H), 3.88 (s, 3H), 3.52 (q, J=7.2 Hz, 2H), 3.36 (q, J=7.2 Hz, 2H), 2.43 (s, 3H), 1.25 (t, J=7.2 Hz, 3H), 1.17 (t, J=7.2 Hz, 3H). ¹³C NMR (101 MHZ, CDCl₃): δ 186.2, 167.9, 156.8, 134.9, 131.2, 125.7, 122.6, 115.2, 114.6, 100.7, 55.7, 42.5, 39.1, 16.5, 14.3, 12.8.

### Step 2: N,N-diethyl-2-(5-methoxy-7-methyl-1H-indol-3-yl)ethan-1-amine (P-54)

To an ice-cold stirred solution of N, N-diethyl-2-(5-methoxy-7-methyl-1H-indol-3-yl)-2-oxoacetamide (489 mg, 1.70 mmol) in anhydrous THF (15 mL) was added LiAlH₄ (193 mg, 5.09 mmol) in portions. The resulting suspension was then heated at reflux for 1 h. The suspension was cooled in an ice bath and quenched by subsequent addition of H₂O (200 μL), 30% NaOH (w/v) (200 μL), H₂O (300 μL). The suspension was stirred at 0° C. for a further 1 h, then Na₂SO₄ was added, and the reaction mass filtered through a pad of celite. The filtrate was concentrated under a stream of N₂ gas to give N,N-diethyl-2-(5-methoxy-7-methyl-1H-indol-3-yl)ethan-1-amine (320 mg) as a colourless oil which was used in the subsequent step without further purification.

### Step 2a: N,N-diethyl-2-(5-methoxy-7-methyl-1H-indol-3-yl)ethan-1-amine fumarate (P-54⋅fumarate)

A solution of N,N-diethyl-2-(5-methoxy-7-methyl-1H-indol-3-yl)ethan-1-amine (310 mg, 1.19 mmol) in hot acetone (2 mL) was added dropwise to a saturated solution of fumaric acid (138 mg, 1.19 mmol) in acetone at reflux. The colorless solution was allowed to cool to ambient temperature and stood at this temperature for 16 h. The resultant colourless needles were collected by vacuum filtration and dried in a vacuum desiccator to afford N, N-diethyl-2-(5-methoxy-7-methyl-1H-indol-3-yl)ethan-1-amine as the fumarate salt (296 mg, 67% over 2 steps). ¹H NMR (400 MHZ, DMSO-d₆): δ 10.68 (s, 1H), 7.13 (d, J=2.4 Hz, 1H), 6.85 (d, J=2.4 Hz, 1H), 6.57-6.51 (m, 1H), 6.49 (s, 1H), 3.74 (s, 3H), 2.98-2.77 (m, 8H), 2.39 (s, 3H), 1.10 (t, J=7.2 Hz, 6H). ¹³C NMR (101 MHZ, DMSO-d₆): δ 168.2, 153.1, 135.2, 131.0, 126.9, 123.0, 121.4, 111.6, 111.5, 97.5, 55.2, 52.1, 45.8, 21.2, 16.7, 10.3. LCMS (ESI+): m/z 261.1 [M+H]⁺. qNMR Purity (ERETIC): 99.8%.

### Example 24: N-ethyl-N-(2-(5-methoxy-7-methyl-1H-indol-3-yl)ethyl)propan-1-amine (P-55)

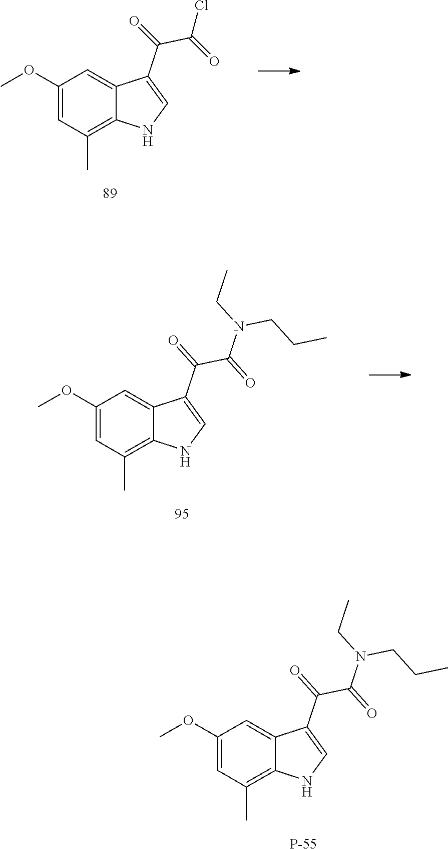

### Step 1: N-ethyl-2-(5-methoxy-7-methyl-1H-indol-3-yl)-2-oxo-N-propylacetamide (95)

A solution of ethyl(propyl)amine (305 mg, 3.5 mmol) in CH₂Cl₂ (5 mL) was added dropwise to an ice-cold suspension of 2-(5-methoxy-7-methyl-1H-indol-3-yl)-2-oxoacetyl chloride (0.4 g, 1.59 mmol) in CH₂Cl₂ (10 mL). Stirring was continued for 3 h at which point the reaction was diluted with H₂O (20 mL) and organic layer separated. The organic layer was washed subsequently with dilute HCl (0.1 M aq, 25 mL), H₂O (2×25 mL) followed by brine (50 mL) before being dried over MgSO₄, filtered, and the filtrate concentrated under reduced pressure. The residue was triturated with CH₂Cl₂/Et₂O and filtered to give crude N-ethyl-2-(5-methoxy-7-methyl-1H-indol-3-yl)-2-oxo-N-propylacetamide (486 mg, quant.) as a white powder.

### Step 2: N-ethyl-N-(2-(5-methoxy-7-methyl-1H-indol-3-yl)ethyl)propan-1-amine (P-55)

To an ice-cold stirred solution of N-ethyl-2-(5-methoxy-7-methyl-1H-indol-3-yl)-2-oxo-N-propylacetamide (481 mg, 1.59 mmol) in anhydrous THF (15 mL) was added LiAlH₄ (181 mg, 4.77 mmol) in portions. The resulting suspension was then heated at reflux for 1 h. The suspension was cooled in an ice bath and quenched by subsequent addition of H₂O (200 μL), 30% NaOH (w/v) (200 μL), H₂O (300 μL). The suspension was stirred at 0° C. for a further 1 h, then Na₂SO₄ was added, and the reaction mass filtered through a pad of celite. The residue was washed with THF (2×20 mL) and the combined filtrates were concentrated under a stream of N₂ gas to give crude N-ethyl-N-(2-(5-methoxy-7-methyl-1H-indol-3-yl)ethyl)propan-1-amine (428 mg) as a colourless oil which was used in the subsequent step without further purification.

### Step 2a: N-ethyl-N-(2-(5-methoxy-7-methyl-1H-indol-3-yl)ethyl)propan-1-amine fumarate (P-55⋅fumarate)

A solution of N-ethyl-N-(2-(5-methoxy-7-methyl-1H-indol-3-yl)ethyl)propan-1-amine (295 mg, 1.08 mmol) in hot acetone (2 mL) was added dropwise to a saturated solution of fumaric acid (125 mg, 1.08 mmol) in acetone at reflux. The colorless solution was allowed to cool to ambient temperature and stood at this temperature for 16 h. The resultant colourless needles were collected by vacuum filtration and dried in a vacuum desiccator to afford N-ethyl-N-(2-(5-methoxy-7-methyl-1H-indol-3-yl)ethyl)propan-1-amine as the fumarate salt (219 mg, 42% over 3 steps). ¹H NMR (400 MHZ, DMSO-d₆): δ 10.65-10.60 (m, 1H), 7.12 (d, J=2.4 Hz, 1H), 6.81 (d, J=2.4 Hz, 1H), 6.54 (d, J=2.4 Hz, 1H), 6.49 (s, 1H), 3.74 (s, 3H), 2.82 (s, 4H), 2.74 (q, J=7.2 Hz, 2H), 2.61 (t, J=7.6 Hz, 2H), 2.39 (s, 3H), 1.51 (m, 2H), 1.06 (t, J=7.2 Hz, 3H), 0.89 (t, J=7.2 Hz, 3H). LCMS (ESI+): m/z 275.2 [M+H]⁺. qNMR Purity (ERETIC): 97.5%.

### Example 25: N-ethyl-N-(2-(5-methoxy-7-methyl-1H-indol-3-yl)ethyl)propan-2-amine (P-56)

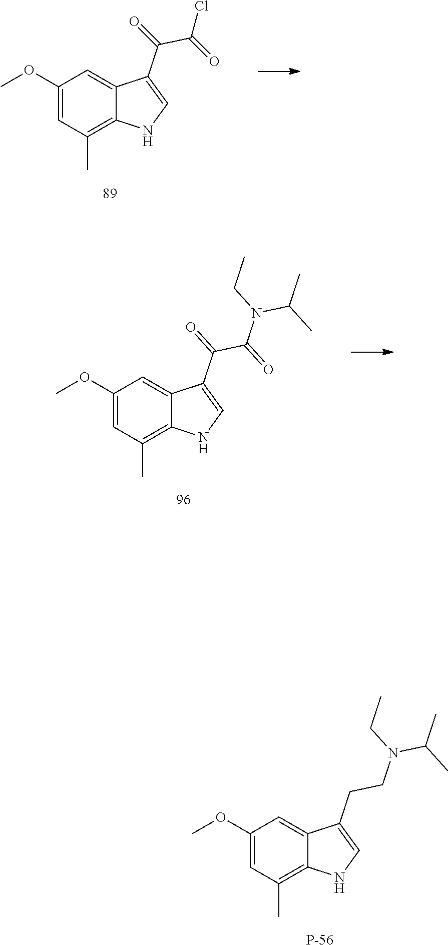

### Step 1: N-ethyl-N-isopropyl-2-(5-methoxy-7-methyl-1H-indol-3-yl)-2-oxoacetamide (96)

To a suspension of the 2-(5-methoxy-7-methyl-1H-indol-3-yl)-2-oxoacetyl chloride (200 mg, 0.80 mmol) in CH₂Cl₂ (5 mL) was added ethyl(propan-2-yl)amine (0.48 mL, 3.97 mmol) at ambient temperature. The reaction was stirred at ambient temperature for 16 h and then diluted with CH₂Cl₂ (10 mL) and dropwise 0.2 M aq. HCl was added until the aqueous layer was acidic. The layers were separated and the organic layer was further washed with 1 M aq. HCl (1 mL×2) and brine (5 mL×1). The organic layer was dried over Na₂SO₄, filtered, the filtrate concentrated, and the residue was purified by flash chromatography (0% to 5% MeOH in CH₂Cl₂, v/v) to afford crude N-ethyl-N-isopropyl-2-(5-methoxy-7-methyl-1H-indol-3-yl)-2-oxoacetamide (157 mg) as a brown solid which was used in the subsequent step without further purification.

### Step 2: N-ethyl-N-(2-(5-methoxy-7-methyl-1H-indol-3-yl)ethyl)propan-2-amine (P-56)

To an ice-cold solution of N-ethyl-N-isopropyl-2-(5-methoxy-7-methyl-1H-indol-3-yl)-2-oxoacetamide (150 mg, 0.47 mmol) in anhydrous THF (10 mL) was added LiAlH₄ (151 mg, 3.97 mmol) in portions under N₂. The reaction was then stirred at ambient temperature for 16 h and then at reflux for 1 h. The suspension was cooled in an ice bath and quenched by subsequent addition of H₂O (150 μL), 30% NaOH (w/v) (150 μL), H₂O (300 μL). The suspension was stirred at 0° C. for a further 15 min, then MgSO₄ was added, and the reaction mass filtered through a pad of celite. The pad was further eluted with several volumes of EtOAc and the combined filtrate concentrated in vacuo. The residue was purified by flash chromatography (0 to 5% MeOH (10% NH_{3(aq)}/CH₂Cl₂, v/v) to afford N-ethyl-N-(2-(5-methoxy-7-methyl-1H-indol-3-yl)ethyl)propan-2-amine (78 mg, 35% over 2 steps) as a lightly coloured oil. 1H NMR (400 MHZ, MeOD-d₄): δ 7.05 (s, 1H), 6.85 (d, J=2.4 Hz, 1H), 6.63-6.57 (m, 1H), 3.80 (s, 3H), 3.29-3.21 (m, 1H), 2.97-2.76 (m, 6H), 2.43 (s, 3H), 1.23-1.09 (m, 9H).

### Step 2a: N-ethyl-N-(2-(5-methoxy-7-methyl-1H-indol-3-yl)ethyl)propan-2-amine hydrochloride (P-56-HCl)

A solution of N-ethyl-N-(2-(5-methoxy-7-methyl-1H-indol-3-yl)ethyl)propan-2-amine (60 mg, 0.22 mmol) in minimal anhydrous i-PrOH under an inert atmosphere (N₂) was cooled to −10° C. and then 0.5 M HCl in anhydrous Et₂O was added dropwise until the pH of the reaction solvent was acidic. The resulting suspension was stirred cold for 15 min and then diluted with anhydrous Et₂O (10 mL) at ambient temperature to promote precipitation. The precipitate was allowed to settle, and solvent removed by decanting. The remaining suspension was triturated with anhydrous Et₂O (1 mL) and the solvent removed by decantation twice, and the solid residue was dried under vacuum to afford N-ethyl-N-(2-(5-methoxy-7-methyl-1H-indol-3-yl)ethyl)propan-2-amine as the hydrochloride salt (23 mg, 38%) which was a white solid. ¹H NMR (400 MHz, D₂O): δ 7.27 (s, 1H), 6.97 (d, J=2.4 Hz, 1H), 6.80-6.70 (m, 1H), 3.86 (s, 3H), 3.70 (sept, J=6.8 Hz, 1H), 3.46-3.31 (m, 1H), 3.30-3.00 (m, 5H), 2.45 (s, 3H), 1.34-1.20 (m, 9H). ¹³C NMR (101 MHZ, D₂O): δ 153.2, 131.3, 126.0, 124.7, 123.4, 112.1, 108.8, 97.9, 56.0, 54.6, 49.0, 45.6, 20.7, 16.0, 15.8, 15.3, 9.4. qNMR Purity (ERETIC): 97.1%.

### Example 26: N, N-dipropyl-2-(5-methoxy-7-methyl-1H-indol-3-yl)ethan-1-amine (P-57)

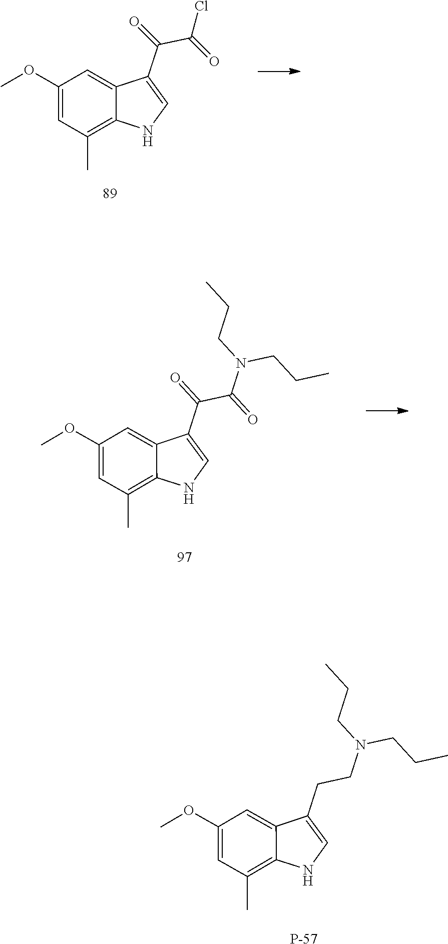

### Step 1: N,N-dipropyl-2-(5-methoxy-7-methyl-1H-indol-3-yl)-2-oxoacetamide (97)

A solution of dipropylamine (354 mg, 3.5 mmol) in CH₂Cl₂ (5 mL) was added dropwise to an ice-cold suspension of 2-(5-methoxy-7-methyl-1H-indol-3-yl)-2-oxoacetyl chloride (0.4 g, 1.59 mmol). Stirring was continued for 3 h at which point the reaction was diluted with H₂O (20 mL) and organic layer separated. The organic layer was washed subsequently with dilute HCl (0.1 M aq., 25 mL), H₂O (2×25 mL) followed by brine (50 mL) before being dried over MgSO₄, filtered, and the filtrate concentrated under reduced pressure. The residue was triturated with CH₂Cl₂/Et₂O and filtered to give N,N-dipropyl-2-(5-methoxy-7-methyl-1H-indol-3-yl)-2-oxoacetamide (340 mg, 68%) as a white powder. ¹H NMR (400 MHZ, MeOD-d₄): δ 7.85 (s, 1H), 7.56 (s, 1H), 6.77-6.74 (m, 1H), 3.85 (s, 3H), 3.53-3.43 (m, 2H), 3.33-3.25 (m, 2H), 2.49 (s, 3H), 1.82-1.57 (m, 4H), 1.02 (t, J=7.4 Hz, 3H), 0.80 (t, J=7.4 Hz, 3H). ¹³C NMR (101 MHZ, MeOD-d₄): δ 186.6, 168.9, 156.9, 135.8, 131.5, 125.7, 122.9, 114.3, 113.8, 100.3, 54.6, 49.6, 45.9, 21.5, 20.2, 15.4, 10.3, 9.9.

### Step 2: N, N-dipropyl-2-(5-methoxy-7-methyl-1H-indol-3-yl)ethan-1-amine (P-57)

To an ice-cold stirred solution of N,N-dipropyl-2-(5-methoxy-7-methyl-1H-indol-3-yl)-2-oxoacetamide (340 mg, 1.07 mmol) in anhydrous THF (15 mL) was added LiAlH₄ (122 mg, 3.22 mmol) in portions. The resulting suspension was then heated at reflux for 1 h. The suspension was cooled in an ice bath and quenched by subsequent addition of H₂O (100 μL), 30% NaOH (w/v) (100 μL), H₂O (300 μL). The suspension was stirred at 0° C. for a further 1 h, then Na₂SO₄ was added, and the reaction mass filtered through a pad of celite. The filter cake was washed with twice with THF (2×20 mL) and the combined filtrates were then concentrated under a stream of N₂ gas to give N, N-dipropyl-2-(5-methoxy-7-methyl-1H-indol-3-yl)ethan-1-amine (310 mg) as a colourless oil which was used in the subsequent step without further purification.

### Step 2a: N, N-dipropyl-2-(5-methoxy-7-methyl-1H-indol-3-yl)ethan-1-amine fumarate (P-57⋅fumarate)

A solution of N,N-dipropyl-2-(5-methoxy-7-methyl-1H-indol-3-yl)ethan-1-amine (186 mg, 0.65 mmol) in hot acetone (2 mL) was added dropwise to a saturated solution of fumaric acid (75 mg, 0.65 mmol) in acetone at reflux. The colorless solution was allowed to cool to ambient temperature and stood at this temperature for 16 h. The resultant colourless needles were collected by vacuum filtration and dried in a vacuum desiccator to afford N, N-dipropyl-2-(5-methoxy-7-methyl-1H-indol-3-yl)ethan-1-amine as the fumarate salt (172 mg, 47% over 2 steps). ¹H NMR (400 MHz, DMSO-d₆): δ 10.68-10.63 (m, 1H), 7.12 (d, J=2.4 Hz, 1H), 6.83 (d, J=2.4 Hz, 1H), 6.56-6.52 (m, 1H), 6.52 (s, 1H), 3.74 (s, 3H), 2.86 (s, 4H), 2.70-2.62 (m, 4H), 2.39 (s, 3H), 1.60-1.46 (m, 4H), 0.89 (t, J=7.2 Hz, 6H). ¹³C NMR (101 MHZ, DMSO-d₆): δ 168.1, 153.6, 135.4, 131.5, 127.4, 123.4, 121.9, 112.3, 112.1, 98.0, 55.7, 55.1, 54.0, 22.0, 19.3, 17.2, 12.1. LCMS (ESI+): m/z 289.2 [M+H]⁺. qNMR Purity (ERETIC): 99.5%.

### Example 27: N-isopropyl-N-(2-(5-methoxy-7-methyl-1H-indol-3-yl)ethyl)propan-2-amine (P-58)

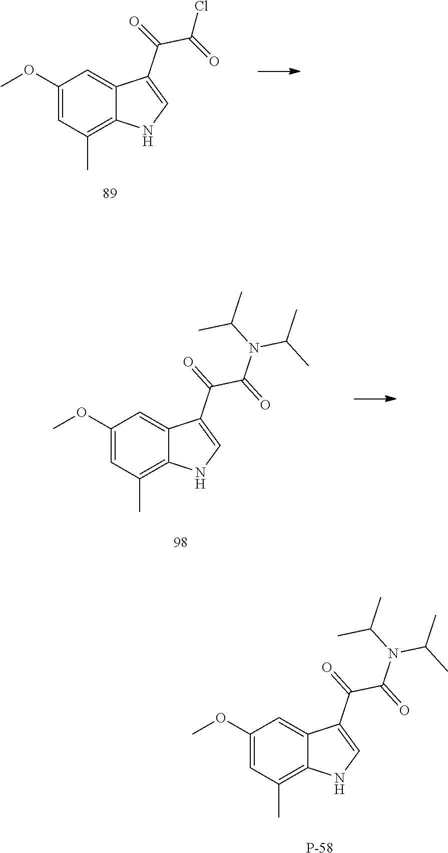

### Step 1: N,N-diisopropyl-2-(5-methoxy-7-methyl-1H-indol-3-yl)-2-oxoacetamide (98)

A solution of bis(propan-2-yl)amine (804 mg, 7.95 mmol) in CH₂Cl₂ (5 mL) was added dropwise to an ice-cold suspension of 2-(5-methoxy-7-methyl-1H-indol-3-yl)-2-oxoacetyl chloride (0.4 g, 1.59 mmol). Stirring was continued for 3 h at which point the reaction was diluted with H₂O (20 mL) and organic layer separated. The organic layer was washed subsequently with dilute HCl (0.1 M aq., 25 mL), H₂O (2×25 mL) followed by brine (50 mL) before being dried over MgSO₄, filtered, and the filtrate concentrated under reduced pressure. The residue was triturated with CH₂Cl₂/Et₂O and filtered to give N,N-diisopropyl-2-(5-methoxy-7-methyl-1H-indol-3-yl)-2-oxoacetamide (358 mg, 71%) as a white powder. ¹H NMR (400 MHZ, CDCl₃): δ 10.72 (s, 1H), 7.56 (d, J=2.4 Hz, 1H), 7.47 (d, J=3.3 Hz, 1H), 6.65-6.62 (m, 1H), 3.93 (sept, J=6.8 Hz, 1H), 3.85 (s, 3H), 3.54 (sept, J=6.8 Hz, 1H), 2.34 (s, 3H), 1.54 (d, J=6.8 Hz, 6H), 1.13 (d, J=6.8 Hz, 6H).

### Step 2: N-isopropyl-N-(2-(5-methoxy-7-methyl-1H-indol-3-yl)ethyl)propan-2-amine (P-58)

To an ice-cold stirred solution of N,N-diisopropyl-2-(5-methoxy-7-methyl-1H-indol-3-yl)-2-oxoacetamide (455 mg, 1.44 mmol) in anhydrous THF (15 mL) was added LiAlH₄ (164 mg, 4.31 mmol) in portions. The resulting suspension was then heated at reflux for 1 h. The suspension was cooled in an ice bath and quenched by subsequent addition of H₂O (150 μL), 30% NaOH (w/v) (150 μL), H₂O (450 μL). The suspension was stirred at 0° C. for a further 1 h, then Na₂SO₄ was added, and the reaction mass filtered through a pad of celite. The filter cake was washed twice with THF (2×20 mL) and combined filtrates were concentrated under a stream of N₂ gas to give N-isopropyl-N-(2-(5-methoxy-7-methyl-1H-indol-3-yl)ethyl)propan-2-amine (358 mg) as a colourless oil which was used in the subsequent step without further purification.

### Step 2a: N-isopropyl-N-(2-(5-methoxy-7-methyl-1H-indol-3-yl)ethyl)propan-2-amine fumarate (P-58⋅fumarate)

A solution of N-isopropyl-N-(2-(5-methoxy-7-methyl-1H-indol-3-yl)ethyl)propan-2-amine (358 mg, 1.24 mmol) in hot acetone (2 mL) was added dropwise to a saturated solution of fumaric acid (144 mg, 1.24 mmol) in acetone at reflux. The colorless solution was allowed to cool to ambient temperature and stood at this temperature for 16 h. The resultant colourless needles were collected by vacuum filtration and dried in a vacuum desiccator to afford N-isopropyl-N-(2-(5-methoxy-7-methyl-1H-indol-3-yl)ethyl)propan-2-amine as the fumarate salt (379 mg, 75% over 2 steps). ¹H NMR (400 MHZ, DMSO-d₆): δ 10.74 (d, J=2.4 Hz, 1H), 7.20 (d, J=2.4 Hz, 1H), 6.82 (d, J=2.4 Hz, 1H), 6.60-6.54 (m, 3H), 3.74 (s, 3H), 3.46 (sept, J=6.8 Hz, 2H), 3.01-2.91 (m, 4H), 2.40 (s, 3H), 1.22 (d, J=6.8 Hz, 12H). ¹³C NMR (101 MHZ, DMSO-d₆): δ 167.7, 153.7, 135.3, 131.5, 127.3, 123.7, 122.1, 112.1, 111.7, 97.9, 55.7, 51.7, 47.0, 25.3, 19.3, 17.2. LCMS (ESI+): m/z 289.2 [M+H]⁺. qNMR Purity (ERETIC): 95.3%.

Scheme 7: Compounds of general formula (I) can be synthesised from the appropriately substituted indole following the outlined sequence of steps in Scheme 7 or similar as one skilled in the art may consider. An appropriately substituted indole was protected to allow base mediated C-1 methylation followed by deprotection generating intermediate 28.2. From this decorated indole, a similar sequence of synthetic transformations as outlined in Scheme 6 could be utilised to generate the desired compounds. For example, glyoxamide formation by sequential treatment with oxalyl chloride followed by an appropriately substituted amine provides glyoxamide intermediates which when subjected to reductive conditions gave compounds of general formula (I) (exemplified by P-8). One skilled in the art will recognise that utilising differentially substituted amines would allow access to compounds of general formula (I) disclosed herein.

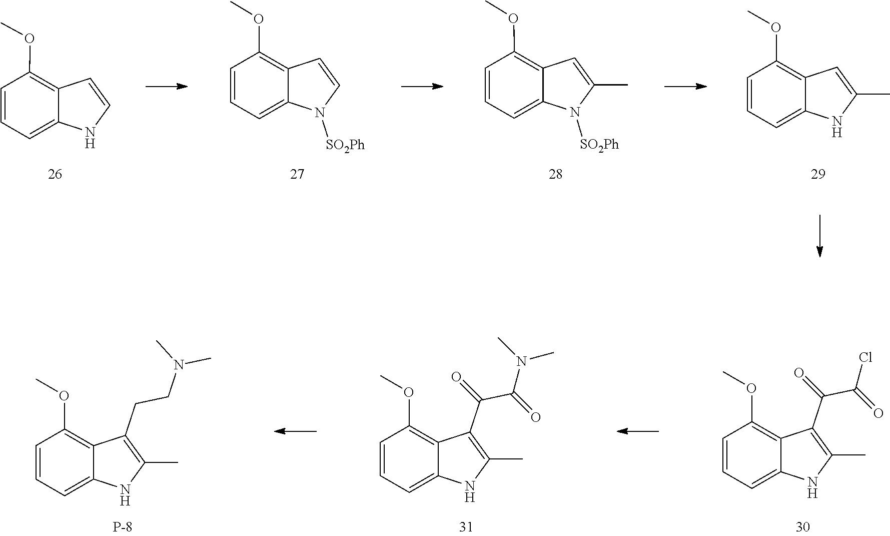

### Example 28: 2-(4-methoxy-2-methyl-1H-indol-3-yl)-N,N-dimethylethan-1-amine (P-8)

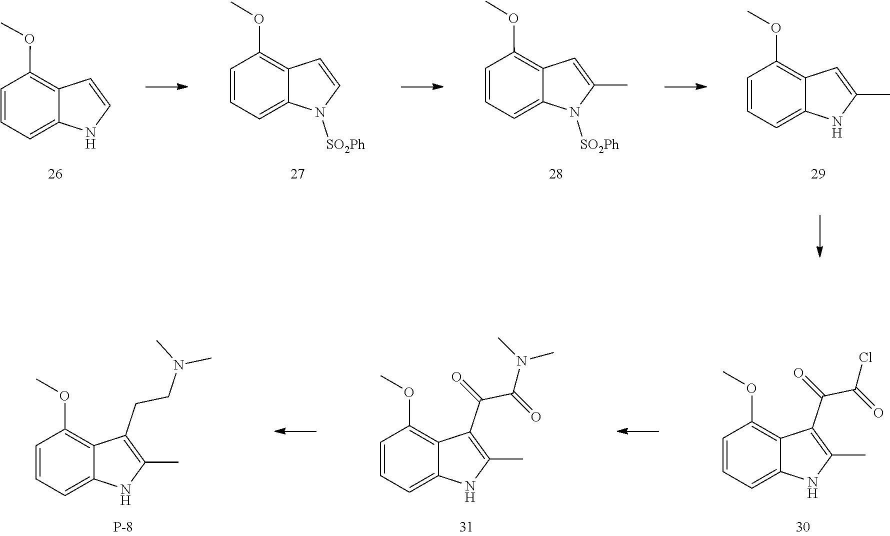

### Step 1: 4-methoxy-1-(phenylsulfonyl)-1H-indole (27)

To a solution of 4-methoxy-1H-indole (4.00 g, 27.2 mmol) in THF (20 mL) and H₂O (4 mL) was added TBAB (876 mg, 2.72 mmol) and NaOH (2.5 M aq., 40 mL) at 20° C., and the reaction mixture stirred for 20 min. Then, a solution of phenylsulfonyl chloride (9.60 g, 54.4 mmol) dissolved in THF (4 mL) was added dropwise at 20° C. The reaction mixture was stirred at 20° C. for 10 h. The reaction was quenched with H₂O (100 mL) and extracted with EtOAc (100 mL×2). The combined organic layers were washed with brine (50 mL), dried over anhydrous Na₂SO₄, filtered, and the filtrate concentrated in vacuo to give crude 4-methoxy-1-(phenylsulfonyl)-1H-indole (14.8 g) as an off-white solid which was used in the subsequent step without further purification.

### Step 2: 4-methoxy-2-methyl-1-(phenylsulfonyl)-1H-indole (28)

To a solution of crude 4-methoxy-1-(phenylsulfonyl)-1H-indole (5.00 g, 17.4 mmol) in THF (25 mL) was added t-BuLi (1.3 M, 17.4 mL) at −30° C. under N₂. The reaction mixture was stirred at −30° C. for 30 min and then, Mel (7.41 g, 52.2 mmol) was added at −30° C. The reaction mixture was then warmed to 25° C. and stirring continued for 4 h. The mixture was quenched with saturated NH₄Cl aqueous solution (50 mL) and then extracted with EtOAc (25 mL×2). The combined organic layers were washed with brine (20 mL), dried over anhydrous Na₂SO₄, filtered and the filtrate concentrated. The residue was purified by column chromatography (SiO₂, 1-6% EtOAc in petroleum ether) to give 4-methoxy-2-methyl-1-(phenylsulfonyl)-1H-indole (4.20 g, 51% over 2 steps) as an off-white solid. ¹H NMR (400 MHZ, CDCl₃): δ 7.78 (d, J=8.0 Hz, 3H), 7.53 (t, J=7.6 Hz, 1H), 7.42 (d, J=8.0 Hz, 2H), 7.19 (t, J=8.0 Hz, 1H), 6.66 (d, J=8.0 Hz, 1H), 6.48 (s, 1H), 3.89 (s, 3H), 2.60 (s, 3H).

### Step 3: 4-methoxy-2-methyl-1H-indole (29)

To a solution of 4-methoxy-2-methyl-1-(phenylsulfonyl)-1H-indole (3.95 g, 13.1 mmol) in EtOH (25 mL) was added NaOH (3.0 M aq., 98.7 mL). The reaction mixture was stirred at 90° C. for 24 h. The mixture was quenched by H₂O (70 mL) and extracted with CH₂Cl₂ (50 mL). The organic layer was washed with brine (30 mL), dried over anhydrous Na₂SO₄, filtered, and the filtrate concentrated under reduced pressure. The residue was purified by column chromatography (SiO₂, 1-20% EtOAc in petroleum ether) to give 4-methoxy-2-methyl-1H-indole (1.41 g, 67% yield) as a purple solid. 1H NMR (400 MHZ, CDCl₃): δ 7.86 (br s, 1H), 7.05 (t, J=8.0 Hz, 1H), 6.94 (d, J=8.0 Hz, 1H), 6.56-6.51 (m, 1H), 6.32 (s, 1H), 3.95 (s, 3H), 2.45 (s, 3H).

### Step 4: 2-(4-methoxy-2-methyl-1H-indol-3-yl)-2-oxoacetyl chloride (30)

To a solution of 4-methoxy-2-methyl-1H-indole (0.90 g, 5.58 mmol) in THF (6 mL) was added (COCl)₂ (1.06 g, 8.37 mmol) at 0° C. under N₂. The reaction mixture was stirred at 0° C. for 2 h. The reaction mixture was used into the next step without further isolation or purification.

### Step 5: 2-(4-methoxy-2-methyl-1H-indol-3-yl)-N,N-dimethyl-2-oxoacetamide (31)

A freshly prepared solution of 2-(4-methoxy-2-methyl-1H-indol-3-yl)-2-oxoacetyl chloride (1.41 g, 5.60 mmol) dissolved in THF (6 mL) was added dropwise into Me₂NH (2.0 M in THF, 3.50 mL) at 0° C. The reaction mixture was stirred at 25° C. for 12 h. The mixture was concentrated under reduced pressure. The residue was purified by column chromatography (SiO₂, 10%-100% EtOAc in petroleum ether) to give 2-(4-methoxy-2-methyl-1H-indol-3-yl)-N,N-dimethyl-2-oxoacetamide (1.00 g, 69% yield) as a yellow solid. ¹H NMR (400 MHZ, MeOD-d₄): δ 7.12 (t, J=8.0 Hz, 1H), 6.99 (d, J=8.0 Hz, 1H), 6.69 (d, J=8.0 Hz, 1H), 3.86 (s, 3H), 3.10 (s, 3H), 3.08 (s, 3H), 2.68 (s, 3H).

### Step 6: 2-(4-methoxy-2-methyl-1H-indol-3-yl)-N,N-dimethylethan-1-amine (P-8)

To a solution of 2-(4-methoxy-2-methyl-1H-indol-3-yl)-N,N-dimethyl-2-oxoacetamide (520 mg, 2.00 mmol) in THF (5 mL) was added LiAlH₄ (910 mg, 24.0 mmol) in portions under N₂. The reaction mixture was stirred at 70° C. for 3 h. The mixture was quenched by adding Na₂SO₄·10H₂O (2.00 g) in portions at 0° C. The mixture was then filtered, and the filter cake was washed with THF (20 mL). The combined filtrate was evaporated and the residue was purified by preparative HPLC (column: Phenomenex C18 (75×30 mm×3 μm; mobile phase: [water (NH₄HCO₃)-ACN]; B: 1-30%, 8 min) to give 2-(4-methoxy-2-methyl-1H-indol-3-yl)-N,N-dimethylethan-1-amine (95.4 mg, 20% yield) as a pink solid. ¹H NMR (400 MHZ, MeOD-d₄): δ 6.90 (t, J=8.0 Hz, 1H), 6.83 (d, J=8.0 Hz, 1H), 6.42 (d, J=7.6 Hz, 1H), 3.88 (s, 3H), 3.01-2.97 (m, 2H), 2.63-2.59 (m, 2H), 2.41 (s, 6H), 2.31 (s, 3H). LCMS (ESI+): m/z 233.1 [M+H]⁺. HPLC Purity (220 nm): 97.6%.

Scheme 8: Compounds of general formula (I) can be synthesised from the appropriately substituted indole following the outlined sequence of steps in Scheme 8 or similar as one skilled in the art may consider. An appropriately substituted toluene could be nitrated in the ortho-position, standard functional group transformations were suitable for the installation of the methoxy functionality via diazonium salt formation. One skilled in the art will recognise that alternate diazonium transformations could be initiated at this stage to install a range of functional groups. Following this, a modified version of the Leimgruber-Batcho indole synthesis via conversion to an appropriately substituted styrene allowed access to the indole intermediates. Glyoxylation of such indoles with oxalyl chloride followed by treatment with an appropriately substituted amine gave glyoxamide intermediates which when subjected to reductive conditions provides access to compounds of general formula (I) (exemplified by P-9). Subsequent demethylation of such compounds allows access to compounds of general formula (I) (exemplified by P-10). One skilled in the art will recognise that utilising differentially substituted amines would allow access to compounds of general formula (I) disclosed herein.

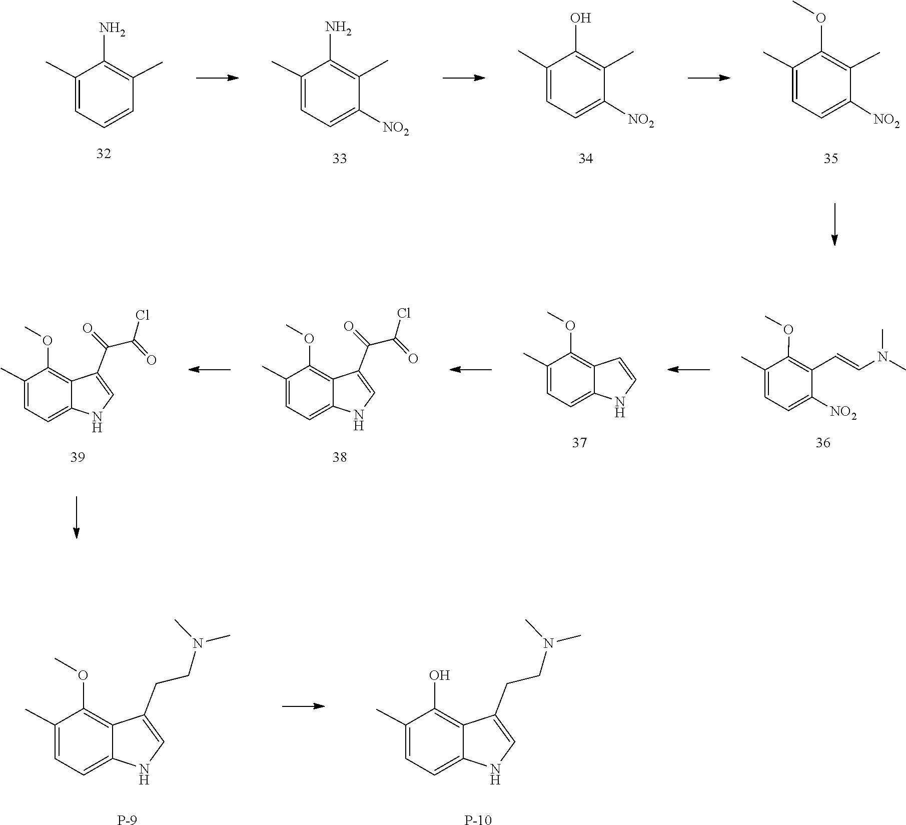

### Example 29: 2-(4-methoxy-5-methyl-1H-indol-3-yl)-N,N-dimethylethan-1-amine (P-10)

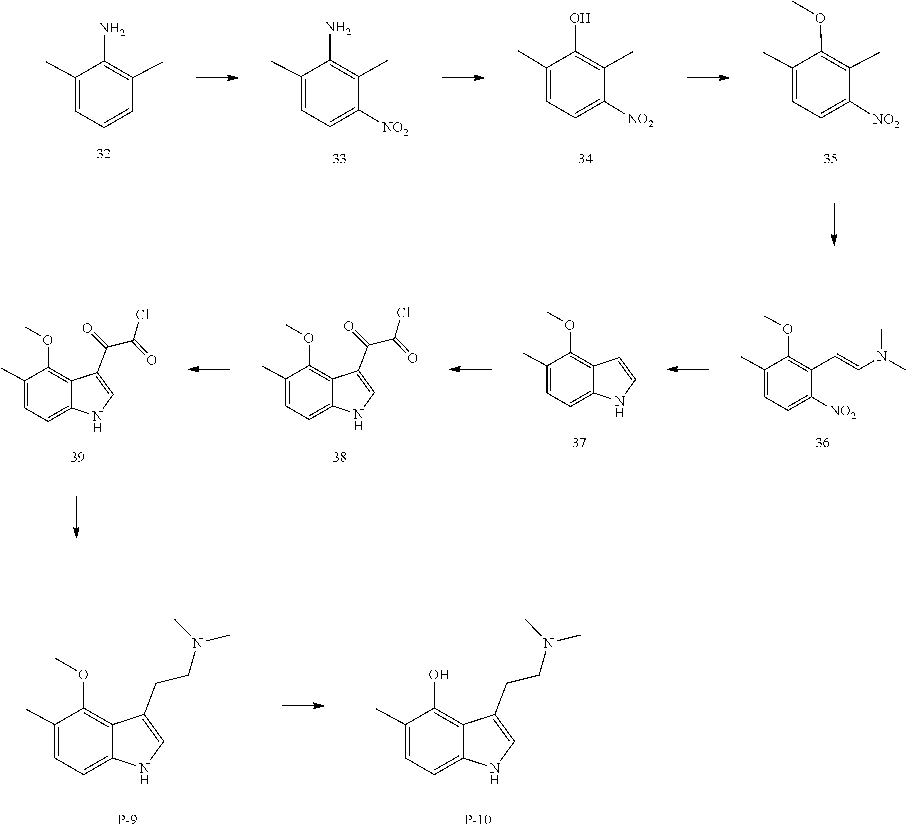

### Step 1: 2,6-dimethyl-3-nitroaniline (33)

2,6-Dimethylaniline (45.0 g, 371 mmol) was added slowly into H₂SO₄ (270 mL), the mixture was cooled to 0-10° C., and fuming HNO₃ (25.9 g, 390 mmol) was added dropwise. The reaction mixture was stirred at 0-10° C. for 1 h. The mixture was quenched by pouring into ice-water (1 L) and the pH was adjusted to 7-8 with 6 N aqueous NaOH solution. The precipitate was filtered, and the filter cake was washed with H₂O (500 mL×2). The filter cake was dried under vacuum and azeotroped with toluene (1 L×3) to give 2,6-dimethyl-3-nitroaniline as a brown solid (58.5 g, 95% yield). ¹H NMR (400 MHZ, CDCl₃): δ 7.17 (d, J=8.0 Hz, 1H), 7.01 (d, J=8.0 Hz, 1H), 3.87 (s, 2H), 2.29 (s, 3H), 2.23 (s, 3H).

### Step 2: 2,6-dimethyl-3-nitrophenol (34)

To an ice-cold (0° C.) stirred solution of 2,6-dimethyl-3-nitroaniline (30.7 g, 185 mmol) in H₂O (263 mL) was added sequentially; H₂SO₄ (132 mL) dropwise followed by a solution of NaNO₂ (16.6 g, 240 mmol) dissolved in H₂O (44 mL) and stirring continued for a further 30 min at this temperature. Another solution of H₂SO₄ (11 mL) in H₂O (102 mL) was heated to 105° C. and the preceding reaction mixture was added at 105° C. The reaction mixture was stirred at 105° C. for 15 min. The mixture was allowed to cool to 25° C. and the aqueous phase was extracted with EtOAc (700 mL×3). The combined organic layers were washed with brine (500 mL), dried over anhydrous Na₂SO₄, filtered and the filtrate concentrated. The residue was purified by column chromatography (SiO₂, 2-6% EtOAc in petroleum ether) to give 2,6-dimethyl-3-nitrophenol (22.0 g, 71% yield) as a yellow solid. ¹H NMR: (400 MHZ, CDCl₃): δ 7.42 (d, J=8.0 Hz, 1H), 7.08 (d, J=8.0 Hz, 1H), 5.11 (s, 1H), 2.43 (s, 3H), 2.32 (s, 3H).

### Step 3: 2-methoxy-1,3-dimethyl-4-nitrobenzene (35)

To a solution of 2,6-dimethyl-3-nitrophenol (66.0 g, 395 mmol) in DMF (462 mL) was added K₂CO₃ (70.9 g, 513 mmol). The reaction mixture was stirred at 25° C. for 30 min and then Mel (112 g, 790 mmol) was added. The reaction mixture was stirred at 25° C. for 12 h. The mixture was diluted with H₂O (1 L) and extracted with EtOAc (600 mL×3). The combined organic layers were washed with brine (500 mL), dried over anhydrous Na₂SO₄, filtered and the filtrate concentrated. The residue was purified by column chromatography (SiO₂, 0-2% EtOAc in petroleum ether) to give 2-methoxy-1,3-dimethyl-4-nitrobenzene (64.0 g, 89% yield) as a pale yellow oil. ¹H NMR (400 MHz, CDCl₃): δ 7.63 (d, J=8.4 Hz, 1H), 7.14 (d, J=8.4 Hz, 1H), 3.75 (s, 3H), 2.49 (s, 3H), 2.36 (s, 3H).

### Step 4: (E)-1-(2-methoxy-3-methyl-6-nitrostyryl)pyrrolidine (36)

To a solution of 2-methoxy-1,3-dimethyl-4-nitrobenzene (18.9 g, 104 mmol) in DMF (209 mL) was added DMF-DMA (13.7 g, 115 mmol) and pyrrolidine (8.90 g, 125 mmol). The reaction mixture was stirred at 125° C. for 3 h under N₂. The solvent was evaporated and crude (E)-1-(2-methoxy-3-methyl-6-nitrostyryl)pyrrolidine (24.7 g) was obtained as a dark oil that was used in the next step without further purification.

### Step 5: 4-methoxy-5-methyl-1H-indole (37)

To a solution of crude (E)-1-(2-methoxy-3-methyl-6-nitrostyryl)pyrrolidine (24.7 g) in toluene (172 mL) and AcOH (78 mL) was added Fe (104 g, 1.87 mol). The reaction mixture was stirred at 110° C. for 2.5 h. The mixture was filtered, and the filter cake was washed with EtOAc (800 mL). The filtrate was evaporated. The residue was diluted with EtOAc (800 mL) and saturated aqueous NaHCO₃ solution was added until the pH of the aqueous layer was 8. The layers were separated, and the organic layer was further washed with brine (200 mL) before being dried over anhydrous Na₂SO₄, filtered, and the filtrate concentrated. The residue was purified by column chromatography (SiO₂, 1-5% EtOAc in petroleum ether) to give 4-methoxy-5-methyl-1H-indole (2.00 g, 12% yield) as a colourless oil. ¹H NMR (400 MHZ, CDCl₃): δ 8.11 (br s, 1H), 7.16-7.14 (m, 1H), 7.06 (d, J=8.0 Hz, 1H), 7.00 (d, J=8.0 Hz, 1H), 6.65-6.62 (m, 1H), 4.03 (s, 3H), 2.36 (s, 3H).

### Step 6: 2-(4-methoxy-5-methyl-1H-indol-3-yl)-2-oxoacetyl chloride (38)

To a solution of 4-methoxy-5-methyl-1H-indole (1.40 g, 8.68 mmol) in THF (9.80 mL) was added (COCl)₂ (1.65 g, 13.0 mmol) at 0° C. under N₂ and stirring continued for 2 h at 0° C. The solvent was evaporated to give 2-(4-methoxy-5-methyl-1H-indol-3-yl)-2-oxoacetyl chloride (2.19 g) as a brown solid that was used in the next step without further purification.

### Step 7: 2-(4-methoxy-5-methyl-1H-indol-3-yl)-N,N-dimethyl-2-oxoacetamide (39)

2-(4-Methoxy-5-methyl-1H-indol-3-yl)-2-oxoacetyl chloride (2.19 g, 8.70 mmol) was added portionwise into a 2.0 M solution of dimethylamine in THF (40 mL) at 0° C. under N₂. The reaction mixture was stirred at 25° C. for 12 h. The solvent was evaporated and the residue was purified by column chromatography (SiO₂, 10-20% EtOAc in petroleum ether) to afford 2-(4-methoxy-5-methyl-1H-indol-3-yl)-N,N-dimethyl-2-oxoacetamide (870 mg, 38%) as a brown solid. ¹H NMR (400 MHZ, MeOD-d₄): δ 7.97 (s, 1H), 7.21-7.14 (m, 2H), 3.79 (s, 3H), 3.10 (s, 3H), 3.04 (s, 3H), 2.39 (s, 3H). LCMS (ESI+): m/z 261.1 [M+H]⁺.

### Step 8: 2-(4-methoxy-5-methyl-1H-indol-3-yl)-N,N-dimethylethan-1-amine (P-9)

To an ice-cold (0° C.) solution of 2-(4-methoxy-5-methyl-1H-indol-3-yl)-N,N-dimethyl-2-oxoacetamide (870 mg, 3.34 mmol) in THF (6 mL) was added LiAlH₄ (1.52 g, 40.1 mmol). The reaction mixture was stirred at 70° C. for 3 h. The mixture was cooled to 10° C. and quenched by Na₂SO₄·10H₂O (5.00 g). The mixture was filtered, and the filter cake was washed with THF (10 mL), and the combined filtrate evaporated. The residue was purified by preparative HPLC (column: Waters Xbridge BEH C18 100×30 mm×10 μm; mobile phase: [water (NH₄HCO₃)-ACN]; B: 1-40%, 8 min) to give 2-(4-methoxy-5-methyl-1H-indol-3-yl)-N,N-dimethylethan-1-amine (80.7 mg, 10%) as an off-white solid. ¹H NMR (400 MHZ, MeOD-d₄): δ 7.01 (d, J=8.0 Hz, 1H), 6.96 (s, 1H), 6.88 (d, J=8.4 Hz, 1H), 3.82 (s, 3H), 3.06-3.02 (m, 2H), 2.75-2.71 (m, 2H), 2.39 (s, 6H), 2.33 (s, 3H). LCMS (ESI+): m/z 233.1 [M+H]⁺. HPLC Purity (220 nm): 97.1%.

### Step 9: 3-(2-(dimethylamino)ethyl)-5-methyl-1H-indol-4-ol (P-10)

To a solution of 2-(4-methoxy-5-methyl-1H-indol-3-yl)-N,N-dimethylethan-1-amine (400 mg, 1.72 mmol) in CH₂Cl₂ (10 mL) was added AlCl₃ (1.38 g, 10.3 mmol) and EtSH (1.93 g, 31.0 mmol) at 0° C. under N₂. The reaction mixture was stirred at 25° C. for 1 h. The mixture was quenched by saturated aqueous NaHCO₃ solution (10 mL), and the aqueous phase was extracted with CH₂Cl₂ (10 mL×3). The combined organic layers were dried over Na₂SO₄, filtered, and the filtrate concentrated. The residue was purified by preparative HPLC (column: Waters Xbridge BEH C18 100×30 mm×10 μm; mobile phase: [water (TFA)-ACN]; B: 1-25%, 8 min) to give 3-(2-(dimethylamino)ethyl)-5-methyl-1H-indol-4-ol as the trifluoroacetate salt (94.1 mg, 16%) which was a brown solid. ¹H NMR (400 MHZ, MeOD-d₄): δ 7.01 (s, 1H), 6.85-6.79 (m, 2H), 3.52 (t, J=7.2 Hz, 2H), 3.30-3.27 (m, 2H), 2.92 (s, 6H), 2.28 (s, 3H). LCMS (ESI+): m/z 219.1 [M+H]⁺. HPLC Purity (220 nm): 99.2%.

### Example 30: N-ethyl-2-(4-methoxy-5-methyl-1H-indol-3-yl)-N-methylethan-1-amine (P-59)

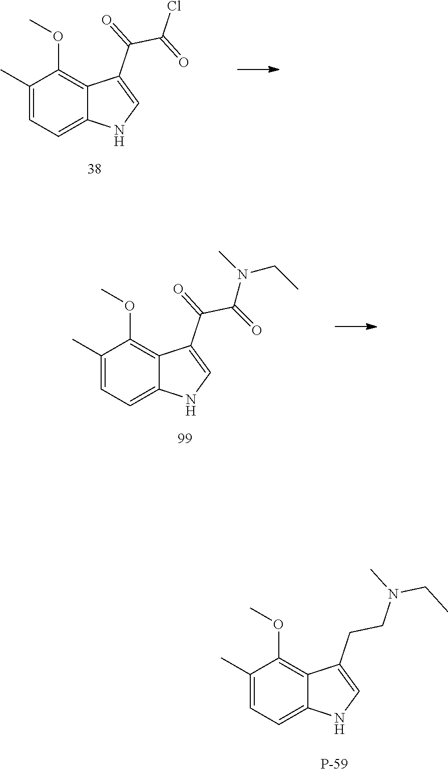

### Step 1: N-ethyl-2-(4-methoxy-5-methyl-1H-indol-3-yl)-N-methyl-2-oxoacetamide (99)

To a solution of 2-(4-methoxy-5-methyl-1H-indol-3-yl)-2-oxoacetyl chloride (702 mg, 2.79 mmol) in THF (7 mL) at 0° C. was added N-methylethan-1-amine (1.65 g, 27.9 mmol) in THF (5 mL). The reaction mixture was stirred at 25° C. for 12 h then concentrated in vacuo. The crude product was purified by flash column chromatography (SiO₂, 10-100% EtOAc in petroleum ether) to afford crude N-ethyl-2-(4-methoxy-5-methyl-1H-indol-3-yl)-N-methyl-2-oxoacetamide (800 mg) as a yellow solid which was used in the subsequent step without further purification. LCMS (ESI+): m/z 275.3 [M+H]⁺.

### Step 2: N-ethyl-2-(4-methoxy-5-methyl-1H-indol-3-yl)-N-methylethan-1-amine (P-59)

To an ice-cold (0° C.) solution of crude N-ethyl-2-(4-methoxy-5-methyl-1H-indol-3-yl)-N-methyl-2-oxoacetamide (800 mg) in THF (8 mL) was added LiAlH₄ (1.33 g, 35.0 mmol) portionwise. The reaction mixture was then stirred at 70° C. for 3 h, before being cooled to 10° C., and quenched by the portionwise addition of Na₂SO₄·10H₂O (5 g). The mixture was filtered, the filter cake was washed with THF (10 mL), and the combined filtrate evaporated. The residue was purified by preparative HPLC (column: Phenomenex Gemini NX-C18 75×30 mm×3 μm; mobile phase: [water (0.05% aq. NH₃+10 mM NH₄HCO₃)-ACN]; B: 10-40%, 8 min) to afford N-ethyl-2-(4-methoxy-5-methyl-1H-indol-3-yl)-N-methylethan-1-amine (150 mg, 22% over 2 steps) as a brown solid. ¹H NMR (400 MHZ, MeOD-d₄): δ 7.00 (d, J=8.4 Hz, 1H), 6.95 (s, 1H), 6.88 (d, J=8.0 Hz, 1H), 3.82 (s, 3H), 3.05-3.01 (m, 2H), 2.79-2.75 (m, 2H), 2.60 (q, J=7.2 Hz, 2H), 2.39 (s, 3H), 2.33 (s, 3H), 1.14 (t, J=7.2 Hz, 3H). LCMS (ESI+): m/z 247.3 [M+H]⁺. HPLC Purity (220 nm): 96.3%.

### Example 31: N-(2-(4-methoxy-5-methyl-1H-indol-3-yl)ethyl)-N-methylpropan-2-amine (P-60)

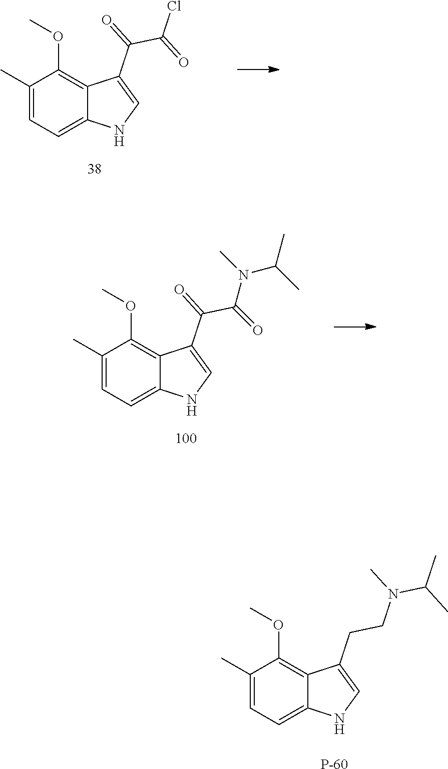

### Step 1: N-isopropyl-2-(4-methoxy-5-methyl-1H-indol-3-yl)-N-methyl-2-oxoacetamide (100)

A solution of 2-(4-methoxy-5-methyl-1H-indol-3-yl)-2-oxoacetyl chloride (781 mg, 3.10 mmol) in THF (7 mL) was added to N-methylpropan-2-amine (2.27 g, 17.6 mmol) at 0° C. under N₂. The reaction mixture was stirred at 25° C. for 12 h, and then the solvent was evaporated. The residue was purified by column chromatography (SiO₂, 20-100% EtOAc in petroleum ether) to afford N-isopropyl-2-(4-methoxy-5-methyl-1H-indol-3-yl)-N-methyl-2-oxoacetamide (750 mg, 84%) as a brown solid which was a mixture of rotamers (A: B, approximately 1:2). ¹H NMR (400 MHZ, MeOD-d₄): δ 7.96 (s, 0.65H, rotamer B), 7.90 (s, 0.35H, rotamer A), 7.25-7.15 (m, 2H), 4.81-4.78 (m, 0.35H, rotamer A), 4.05-3.94 (m, 0.65H, rotamer B), 3.80 (s, 3H), 2.96 (s, 2H, rotamer B), 2.88 (s, 1H, rotamer A), 2.39 (s, 3H), 1.30-1.17 (m, 6H). LCMS (ESI+): m/z 289.3 [M+H]⁺.

### Step 2: N-(2-(4-methoxy-5-methyl-1H-indol-3-yl)ethyl)-N-methylpropan-2-amine (P-60)

To an ice-cold (0° C.) solution of N-isopropyl-2-(4-methoxy-5-methyl-1H-indol-3-yl)-N-methyl-2-oxoacetamide (750 mg, 2.60 mmol) in THF (5.25 mL) was added LiAlH₄ (1.18 g, 31.2 mmol) in portions. The reaction mixture was stirred at 70° C. for 3 h. The mixture was cooled to 10° C. and quenched by the addition of Na₂SO₄·10H₂O (5.00 g). The mixture was filtered, the filter cake was washed with THF (10 mL), and the combined filtrate evaporated. The residue was purified by preparative HPLC (column: Waters Xbridge Prep OBD C18 150×40 mm×10 μm; mobile phase: [H₂O (0.05% NH₃+10 mM NH₄HCO₃)-ACN]; B: 15-45%, 8.0 min) to afford N-(2-(4-methoxy-5-methyl-1H-indol-3-yl)ethyl)-N-methylpropan-2-amine (243 mg, 36%) as a white solid. ¹H NMR (400 MHZ, MeOD-d₄): δ 7.03 (d, J=8.4 Hz, 1H), 6.98 (s, 1H), 6.89 (d, J=8.4 Hz, 1H), 3.82 (s, 3H), 3.06-3.02 (m, 3H), 2.86-2.82 (m, 2H), 2.43 (s, 3H), 2.34 (s, 3H), 1.12 (d, J=6.4 Hz, 6H). LCMS (ESI+): m/z 261.1 [M+H]⁺. HPLC Purity (254 nm): 99.7%.

### Example 32: N, N-diethyl-2-(4-methoxy-5-methyl-1H-indol-3-yl)ethan-1-amine (P-61)

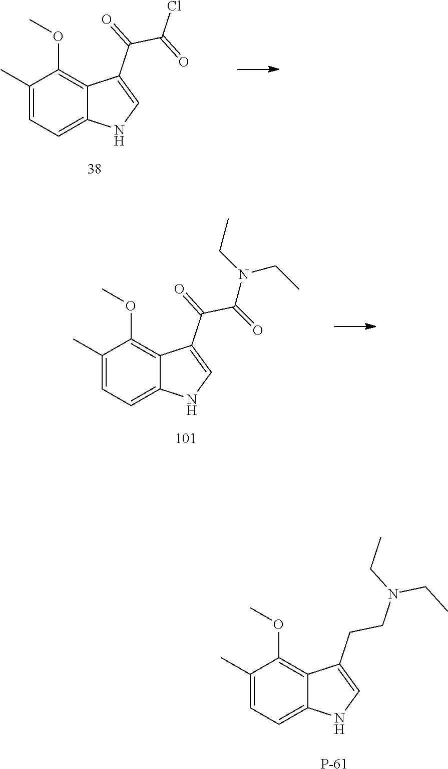

### Step 1: N,N-diethyl-2-(4-methoxy-5-methyl-1H-indol-3-yl)-2-oxoacetamide (101)

A solution of 2-(4-methoxy-5-methyl-1H-indol-3-yl)-2-oxoacetyl chloride (781 mg, 3.1 mmol) in THF (3.5 mL) was added dropwise to diethylamine (2.27 g, 31.0 mmol) at 0° C. under N₂. The reaction mixture was stirred at 25° C. for 12 h. The solvent was evaporated and the residue was purified by column chromatography (SiO₂, 20%-66% EtOAc in petroleum ether) to give crude N,N-diethyl-2-(4-methoxy-5-methyl-1H-indol-3-yl)-2-oxoacetamide (606 mg, 68%) as a brown solid which was used in the subsequent step without further purification. LCMS (ESI+): m/z 289.3 [M+H]⁺.

### Step 2: N,N-diethyl-2-(4-methoxy-5-methyl-1H-indol-3-yl)ethan-1-amine (P-61)

To a solution of crude N,N-diethyl-2-(4-methoxy-5-methyl-1H-indol-3-yl)-2-oxoacetamide (606 mg) in THF (4.24 mL) at 0° C. was added LiAlH₄ (957 mg, 25.2 mmol) in portions and the reaction mixture was stirred at 70° C. for 3 h. The mixture was cooled to 10° C. and quenched by Na₂SO₄·10H₂O (5.00 g). The mixture was filtered, the filter cake was washed with THF (10 mL), and the combined filtrate was evaporated. The residue was purified by preparative HPLC (column: Waters Xbridge Prep OBD C18 150×40 mm×10 μm; mobile phase: [water (0.05% aq. NH₃+10 mM NH₄HCO₃)-ACN]; B: 20-50%, 8.0 min) to give N,N-diethyl-2-(4-methoxy-5-methyl-1H-indol-3-yl)ethan-1-amine (30.5 mg, 4% over 2 steps) as a colourless oil. ¹H NMR (400 MHz, MeOD-d₄): δ 7.01 (d, J=8.0 Hz, 1H), 6.96 (s, 1H), 6.89 (d, J=8.0 Hz, 1H), 3.82 (s, 3H), 3.03-2.99 (m, 2H), 2.88-2.86 (m, 2H), 2.73 (q, J=7.2 Hz, 4H), 2.33 (s, 3H), 1.15 (t, J=7.2 Hz, 6H). LCMS (ESI+): m/z 261.1 [M+H]⁺. HPLC Purity (220 nm): 99.6%.

### Example 33: N-ethyl-N-(2-(4-methoxy-5-methyl-1H-indol-3-yl)ethyl)propan-2-amine (P-62)

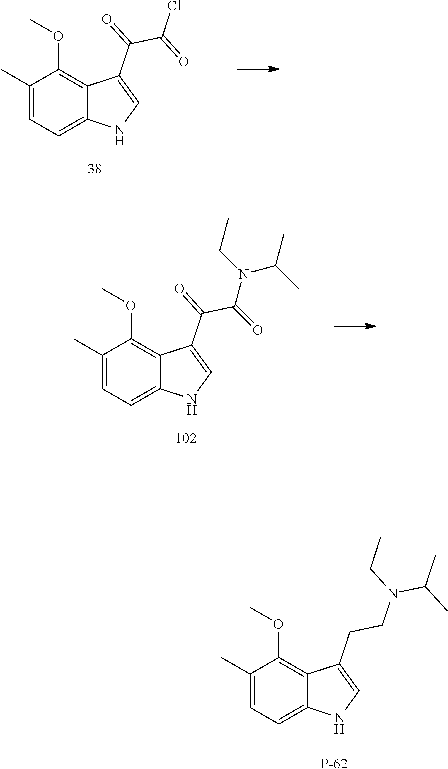

### Step 1: N-ethyl-N-isopropyl-2-(4-methoxy-5-methyl-1H-indol-3-yl)-2-oxoacetamide (102)

A solution of 2-(4-methoxy-5-methyl-1H-indol-3-yl)-2-oxoacetyl chloride (824 mg, 3.28 mmol) in THF (3.5 mL) was added dropwise to N-ethylpropan-2-amine (2.85 g, 32.8 mmol) at 0° C. under N₂. The reaction mixture was stirred at 25° C. for 12 h. The solvent was evaporated and the residue purified by column chromatography (SiO₂, 20-100% EtOAc in petroleum ether) to afford crude N-ethyl-N-isopropyl-2-(4-methoxy-5-methyl-1H-indol-3-yl)-2-oxoacetamide (666 mg) as a brown solid which was used in the subsequent step without further purification. LCMS (ESI+): m/z 303.3 [M+H]⁺.

### Step 2: N-ethyl-N-(2-(4-methoxy-5-methyl-1H-indol-3-yl)ethyl)propan-2-amine (P-62)

To a solution of crude N-ethyl-N-isopropyl-2-(4-methoxy-5-methyl-1H-indol-3-yl)-2-oxoacetamide (666 mg) in THF (4.66 mL) at 0° C. was added LiAlH₄ (1 g, 26.4 mmol) in portions. The reaction mixture was stirred at 70° C. for 3 h, then cooled to 0° C. and quenched with Na₂SO₄·10H₂O (5 g). The mixture was filtered, the filter cake was washed with THF (10 mL), and the combined filtrate was evaporated. The residue was purified by preparative HPLC (column: Waters Xbridge Prep OBD C18 150×40 mm×10 μm; mobile phase: [water (0.05% aq. NH₃+10 mM NH₄HCO₃)-ACN]; B: 20-50%, 8.0 min) to give N-ethyl-N-(2-(4-methoxy-5-methyl-1H-indol-3-yl)ethyl)propan-2-amine (30.1 mg, 3% over 2 steps) as a white solid. ¹H NMR (400 MHZ, MeOD-d₄): δ 7.01 (d, J=8.4 Hz, 1H), 6.96 (s, 1H), 6.89 (d, J=8.4 Hz, 1H), 3.81 (s, 3H), 3.23-3.18 (m, 1H), 3.03-2.98 (m, 2H), 2.85-2.72 (m, 4H), 2.33 (s, 3H), 1.20-1.11 (m, 9H). LCMS (ESI+): m/z 275.1 [M+H]⁺. HPLC Purity (220 nm): 97.6%.

### Example 34: N-isopropyl-N-(2-(4-methoxy-5-methyl-1H-indol-3-yl)ethyl)propan-2-amine (P-63)

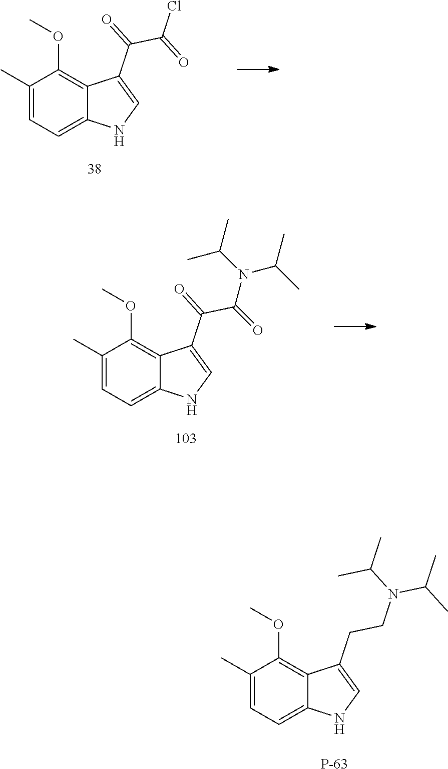

### Step 1: N,N-diisopropyl-2-(4-methoxy-5-methyl-1H-indol-3-yl)-2-oxoacetamide (103)

To a solution of 2-(4-methoxy-5-methyl-1H-indol-3-yl)-2-oxoacetyl chloride (468 mg, 1.86 mmol) in THF (3 mL) at 0° C. was added a solution of N,N-diisopropylamine (1.88 g, 18.6 mmol) in THF (5 mL). The reaction mixture was stirred at 25° C. for 12 h and concentrated under reduced pressure. The residue was purified by column chromatography (SiO₂, 10-100% EtOAc in petroleum ether) to afford N,N-diisopropyl-2-(4-methoxy-5-methyl-1H-indol-3-yl)-2-oxoacetamide (182 mg, 31%) as a yellow solid. ¹H NMR (400 MHZ, MeOD-d₄): δ 7.87 (s, 1H), 7.21-7.15 (m, 2H), 3.97-3.90 (m, 1H), 3.80 (s, 3H), 3.69-3.64 (m, 1H), 2.39 (s, 3H), 1.57 (d, J=6.8 Hz, 6H), 1.21 (d, J=6.8 Hz, 6H). LCMS (ESI+): m/z 317.2 [M+H]⁺.

### Step 2: N-isopropyl-N-(2-(4-methoxy-5-methyl-1H-indol-3-yl)ethyl)propan-2-amine (P-63)

To a solution of N, N-diisopropyl-2-(4-methoxy-5-methyl-1H-indol-3-yl)-2-oxoacetamide (180 mg, 0.57 mmol) in THF (2 mL) was added LiAlH₄ (259 mg, 6.83 mmol) in portions under N₂. The reaction mixture was stirred at 70° C. for 3 h, then cooled to 10° C. and quenched with Na₂SO₄·10H₂O (3.00 g). The reaction mixture was filtered, the filter cake was washed with THF (20 mL), and the combined filtrate was concentrated in vacuo. The residue was purified by preparative HPLC (column: Waters Xbridge Prep OBD C18 150×40 mm×10 μm; mobile phase: [water (0.05% aq. NH₃+10 mM NH₄HCO₃)-ACN]; B: 25-55%, 8.0 min) to afford N-isopropyl-N-(2-(4-methoxy-5-methyl-1H-indol-3-yl)ethyl)propan-2-amine (24 mg, 15%) as an off-white solid. ¹H NMR (400 MHZ, MeOD-d₄): δ 7.07 (d, J=8.4 Hz, 1H), 6.96 (s, 1H), 6.89 (d, J=8.4 Hz, 1H), 3.80 (s, 3H), 3.30-3.16 (m, 2H), 2.98-2.94 (m, 2H), 2.81-2.79 (m, 2H), 2.34 (s, 3H), 1.16 (d, J=6.4 Hz, 12H). LCMS (ESI+): m/z 289.2 [M+H]⁺. HPLC Purity (220 nm): 99.8%

Scheme 9: Compounds of general formula (I) can be synthesised from the appropriately substituted indole following the outlined sequence of steps in Scheme 9 or similar as one skilled in the art may consider. An appropriately substituted indolol was subjected to a regioselective amino methylation in a pseudo-Mannich type reaction to generate intermediate 105 that upon palladium catalysed hydrogenation could provide access to methyl indole intermediate 106. Applying a suitable phenolic protection group strategy, followed by a similar sequence of steps outlined in Scheme 6, namely glyoxamide formation followed by reduction, allowed access to protected intermediates such as 110 which when subjected to deprotection conditions provides access to compounds of general formula (I) (exemplified by P-64). One skilled in the art will recognise that utilising differentially substituted amines during the glyoxamide formation step would allow access to compounds of general formula (I) disclosed herein.

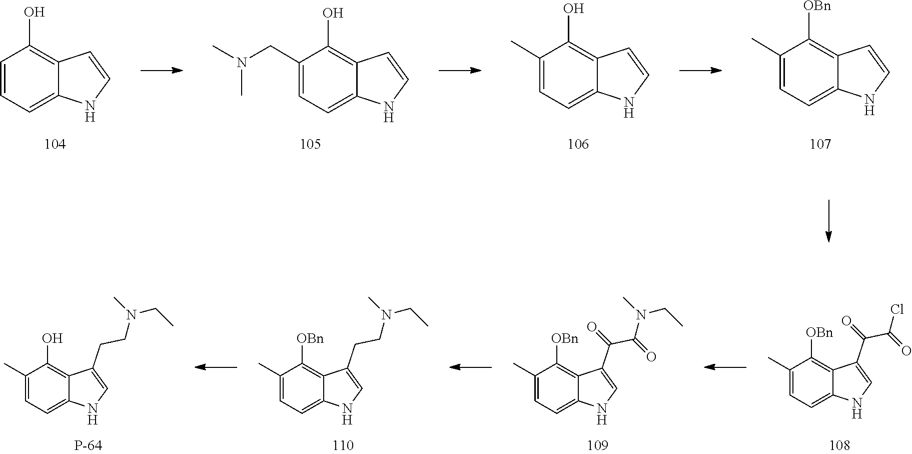

### Example 35: 3-(2-(ethyl(methyl)amino)ethyl)-5-methyl-1H-indol-4-ol (P-64)

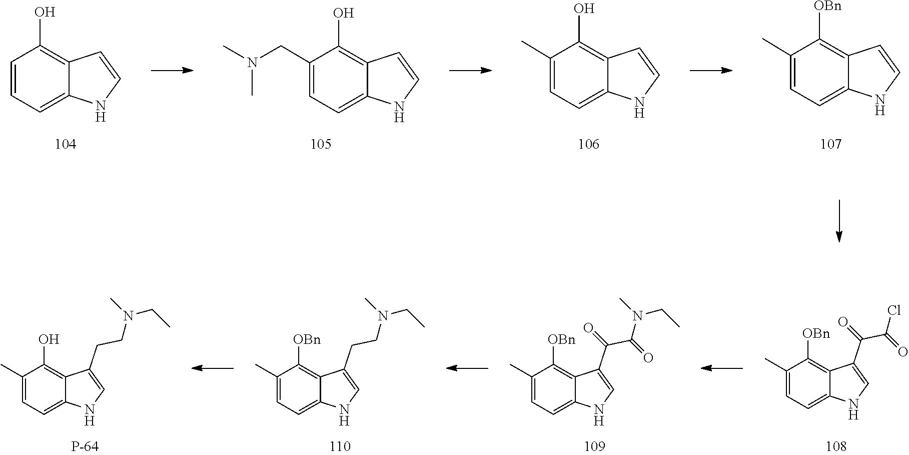

### Step 1: 5-((dimethylamino)methyl)-1H-indol-4-ol (105)

To a stirred solution of 4-hydroxyindole (500 mg, 3.75 mmol) in EtOH (5 mL) was added 40% aqueous dimethylamine (550 mg, 4.87 mmol) and the mixture was treated dropwise with 37% aqueous formaldehyde (350 mg, 4.30 mmol, 1.1 eq). The temperature was raised to 32° C. and then the mixture was stirred at ambient temperature for 1 h. The dark purple solution was diluted with H₂O (20 mL) and extracted with CHCl₃ (3×10 mL). The combined organic layers were dried over Na₂SO₄, filtered, and the filtrate concentrated. The residue was purified by flash chromatography (alumina, 100% EtOAc) to afford 5-((dimethylamino)methyl)-1H-indol-4-ol as a brown oil (310 mg, 43%), which crystallized on standing at 4° C. 1H NMR (400 MHZ, DMSO-d₆): δ 10.95 (br s, 1H), 8.27 (s, 1H), 7.17-7.15 (m, 1H), 6.83-6.72 (m, 2H), 6.50-6.45 (m, 1H), 3.77 (s, 2H), 2.35 (s, 6H). LCMS (ESI+): m/z: 191.1 [M+H]⁺.

### Step 2: 5-methyl-1H-indol-4-ol (106)

A stirred solution of 5-((dimethylamino)methyl)-1H-indol-4-ol (300 mg, 1.57 mmol) in degassed MeOH (20 mL) was treated with palladium on alumina (60 mg) at ambient temperature and the mixture was stirred under an atmosphere of H₂ for 16 h. The mixture was filtered through a celite pad and the filter cake was washed with MeOH (20 mL). The combined filtrate was concentrated under reduced pressure and the residue was purified by column chromatography on (SiO₂, hexane: EtOAc, 8:2, v/v) to afford 5-methyl-1H-indol-4-ol (170 mg, 73%) as a yellow solid. ¹H NMR (400 MHZ, DMSO-d₆): δ 10.75 (br s, 1H), 8.69 (s, 1H), 7.09-7.08 (m, 1H), 6.79-6.72 (m, 2H), 6.56-6.54 (m, 1H), 2.18 (s, 3H).

### Step 3: 4-(benzyloxy)-5-methyl-1H-indole (107)

To a stirred solution of 5-methyl-1H-indol-4-ol (150 mg, 1.02 mmol) in anhydrous DMF (2 mL) was added K₂CO₃ (281 mg, 2.04 mmol) and benzyl bromide (0.14 mL, 1.22 mmol) at ambient temperature and stirred for 16 h. The reaction mixture was quenched with H₂O (25 mL), extracted with EtOAc (2×30 mL). The combined organic layers were washed with brine (20 mL) and dried over Na₂SO₄, filtered, and the filtrate concentrated under reduced pressure. The residue was purified by column chromatography (SiO₂, hexane: EtOAc, 90:10 to 85:15, v/v) to afford 4-(benzyloxy)-5-methyl-1H-indole (190 mg, 79%) as a brown solid. ¹H NMR (400 MHZ, DMSO-d₆): δ 11.04 (br s, 1H), 7.49 (d, J=7.2 Hz, 2H), 7.43-7.30 (m, 3H), 7.26-7.24 (m, 1H), 7.04 (d, J=8.4 Hz, 1H), 6.87 (d, J=8.0 Hz, 1H), 6.50-6.48 (m, 1H), 5.14 (s, 2H), 2.22 (s, 3H); LCMS (ESI+): m/z 238.2 [M+H]⁺.

### Step 4: 2-(4-(benzyloxy)-5-methyl-1H-indol-3-yl)-N-ethyl-N-methyl-2-oxoacetamide (109)

To an ice-cold solution of 4-(benzyloxy)-5-methyl-1H-indole (400 mg, 1.69 mmol) in anhydrous THF (5 mL) was added oxalyl chloride (0.43 mL, 5.04 mmol). The reaction mixture was then stirred at ambient temperature for 2 h. The reaction was cooled again and N-methylethanamine was added dropwise until the pH of the reaction solution was basic, at which point the reaction was stirred for a further 30 min before being quenched with H₂O (25 mL) and extracted with EtOAc (2×50 mL). The combined organic layers were washed with brine (20 mL), and dried over Na₂SO₄, filtered, and the filtrate concentrated under reduced pressure. The residue was purified by column chromatography (SiO₂, hexane: EtOAc, 6:4 to 5:5, v/v) to afford 2-(4-(benzyloxy)-5-methyl-1H-indol-3-yl)-N-ethyl-N-methyl-2-oxoacetamide (600 mg) as a brown solid which was used in the subsequent step without further characterisation. LCMS (ESI+): m/z 373.3 [M+Na]⁺.

### Step 5: 2-(4-(benzyloxy)-5-methyl-1H-indol-3-yl)-N-ethyl-N-methylethan-1-amine (110)

To an ice-cold solution of 2-(4-(benzyloxy)-5-methyl-1H-indol-3-yl)-N-ethyl-N-methyl-2-oxoacetamide (600 mg) in anhydrous THF (20 mL) was added 1 M LiAlH₄, in THF (6.85 mL, 6.85 mmol). The reaction was stirred at 0° C. for 30 min, and then at reflux for 16 h. The reaction mixture was cooled to 0° C. and quenched with saturated aq. Na₂SO₄ solution (3 mL), filtered, and the filtrate concentrated. The residue was diluted with CH₂Cl₂ (25 mL), silica gel (100 mg) was added, and the resulting mixture stirred for 30 min. The resulting slurry was filtered through a celite pad and the filter cake was washed with MeOH (20 mL). The combined filtrates were concentrated under reduced pressure to afford 2-(4-(benzyloxy)-5-methyl-1H-indol-3-yl)-N-ethyl-N-methylethan-1-amine (410 mg) as a purple wax which was used in the subsequent step without further purification. LCMS (ESI+): m/z 323.3 [M+H]⁺.

### Step 6: 3-(2-(ethyl(methyl)amino)ethyl)-5-methyl-1H-indol-4-ol (P-64)

A stirred solution of 2-(4-(benzyloxy)-5-methyl-1H-indol-3-yl)-N-ethyl-N-methylethan-1-amine (400 mg) in degassed MeOH (15 mL) was treated with formic acid (0.3 mL) followed by Pd/C (120 mg, 10% w/w). The reaction was then stirred under H₂ gas at ambient pressure and temperature for 16 h. The reaction mixture was filtered through a celite pad and washed with MeOH (20 mL). The combined filtrate was concentrated under reduced pressure in a bath temperature not exceeding 35° C. The residue was purified by reverse phase column chromatography (C18, 0.01% v/v HCO₂H in MeCN), followed by lyophilization, to afford 3-(2-(ethyl(methyl)amino)ethyl)-5-methyl-1H-indol-4-ol (50 mg, 13% over 3 steps) as a brown solid. ¹H NMR (400 MHZ, DMSO-d₆): δ 10.49 (br. s, 1H), 8.20 (s, 1H), 6.90 (s, 1H), 6.73 (d, J=8.0 Hz, 1H), 6.66 (d, J=8.0 Hz, 1H), 2.96-2.87 (m, 2H), 2.73-2.69 (m, 2H), 2.55 (q, J=7.2 Hz, 2H), 2.34 (s, 3H), 2.16 (s, 3H), 0.99 (t, J=7.2 Hz, 3H). LCMS (ESI+): m/z 233.2 [M+H]⁺. HPLC Purity (PDA): 99.3%

### Example 36: 5-methyl-3-(2-(methyl(propyl)amino)ethyl)-1H-indol-4-ol (P-65)

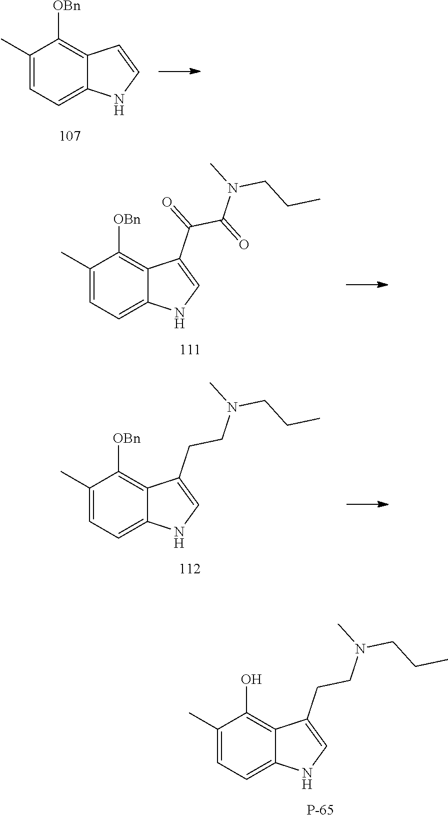

### Step 1: 2-(4-(benzyloxy)-5-methyl-1H-indol-3-yl)-N-methyl-2-oxo-N-propylacetamide (111)

To an ice-cold solution of 4-(benzyloxy)-5-methyl-1H-indole (400 mg, 1.69 mmol) in anhydrous THF (5 mL) was added oxalyl chloride (0.43 mL, 5.04 mmol). The reaction mixture was then stirred at ambient temperature for 2 h. The reaction was cooled again, and N-methylpropan-1-amine was added dropwise until the pH of the reaction solution was basic, at which point the reaction was stirred for 30 min before being quenched with H₂O (25 mL) and extracted with EtOAc (2×50 mL). The combined organic layers were washed with brine (20 mL), dried over Na₂SO₄, filtered, and the filtrate concentrated under reduced pressure. The residue was purified by column chromatography (SiO₂, hexane: EtOAc, 6:4 to 5:5, v/v) to afford 2-(4-(benzyloxy)-5-methyl-1H-indol-3-yl)-N-methyl-2-oxo-N-propylacetamide (650 mg) as a brown oil which was used in the subsequent step without further purification. LCMS (ESI+): m/z 365.3 [M+H]⁺.

### Step 2: N-(2-(4-(benzyloxy)-5-methyl-1H-indol-3-yl)ethyl)-N-methylpropan-1-amine (112)

To an ice-cold solution of 2-(4-(benzyloxy)-5-methyl-1H-indol-3-yl)-N-methyl-2-oxo-N-propylacetamide (600 mg) in anhydrous THF (20 mL) was added 1 M LiAlH₄, in THF (6.59 mL, 6.59 mmol). The reaction was stirred at 0° C. for 30 min, and then at reflux for 16 h. The reaction mixture was cooled to 0° C. and quenched with saturated aq. Na₂SO₄ solution (3 mL), filtered, and the filtrate concentrated. The residue was diluted with CH₂Cl₂ (25 mL), silica gel (100 mg) was added, and the resulting mixture stirred for 30 min. The resulting slurry was filtered through a celite pad, and the filter cake was washed with MeOH (20 mL). The combined filtrate was concentrated under reduced pressure to afford N-(2-(4-(benzyloxy)-5-methyl-1H-indol-3-yl)ethyl)-N-methylpropan-1-amine (390 mg) as a brown oil which was used in the subsequent step without further purification. LCMS (ESI+): m/z 337.3 [M+H]⁺.

### Step 3: 5-methyl-3-(2-(methyl(propyl)amino)ethyl)-1H-indol-4-ol (P-65)

A stirred solution of N-(2-(4-(benzyloxy)-5-methyl-1H-indol-3-yl)ethyl)-N-methylpropan-1-amine (390 mg) in degassed MeOH (15 mL) was treated with formic acid (0.3 mL) followed by Pd/C (120 mg, 10% w/w). The reaction was then stirred under an atmosphere of H₂ gas at ambient pressure and temperature for 16 h. The reaction mixture was filtered through a celite pad and washed with MeOH (20 mL). The combined filtrate was concentrated under reduced pressure. The residue was purified by reverse phase column chromatography (C18, 0.01% v/v HCO₂H in MeCN), followed by lyophilization, to afford 5-methyl-3-(2-(methyl(propyl)amino)ethyl)-1H-indol-4-ol (45 mg, 11% over 3 steps) as a brown solid. ¹H NMR (400 MHZ, DMSO-d₆): δ 10.48 (br s, 1H), 8.20 (s, 1H), 6.90 (d, J=1.6 Hz, 1H), 6.73 (d, J=8.4 Hz, 1H), 6.67 (d, J=8.0 Hz, 1H), 2.89 (t, J=5.6 Hz, 2H), 2.71 (t, J=5.2 Hz, 2H), 2.48-2.42 (m, 2H), 2.35 (s, 3H), 2.16 (s, 3H), 1.43 (sext, J=7.2 Hz, 2H), 0.79 (t, J=7.2 Hz, 3H); LCMS (ESI+): m/z 246.8 [M+H]⁺. HPLC Purity (PDA): 97.9%

### Example 37: 3-(2-(isopropyl(methyl)amino)ethyl)-5-methyl-1H-indol-4-ol (P-66)

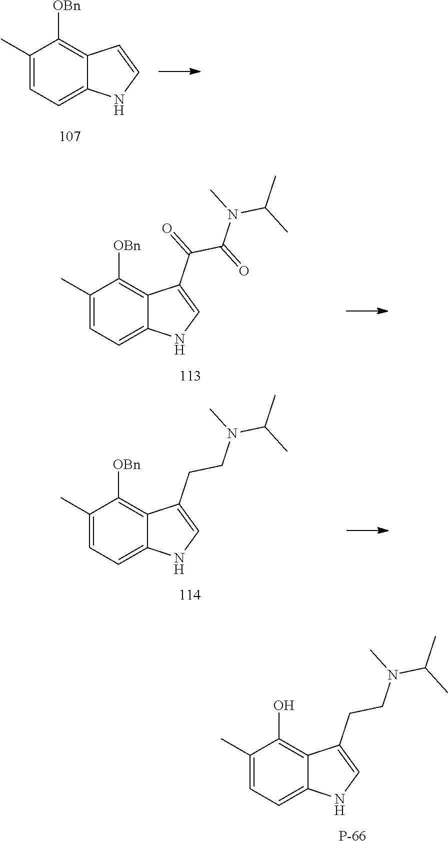

### Step 1: 2-(4-(benzyloxy)-5-methyl-1H-indol-3-yl)-N-isopropyl-N-methyl-2-oxoacetamide (113)

To an ice-cold solution of 4-(benzyloxy)-5-methyl-1H-indole (400 mg, 1.69 mmol) in anhydrous THF (5 mL) was added oxalyl chloride (0.43 mL, 5.04 mmol). The reaction mixture was then stirred at ambient temperature for 2 h. The reaction was cooled again, and N-methyl(isopropyl)amine was added dropwise until the pH of the reaction solution was basic, at which point the reaction was stirred for 30 min before being quenched with H₂O (25 mL) and extracted with EtOAc (2×50 mL). The combined organic layers were washed with brine (20 mL), and dried over Na₂SO₄, filtered, and the filtrate concentrated under reduced pressure. The residue was purified by column chromatography (SiO₂, hexane: EtOAc, 6:4 to 5:5, v/v) to afford 2-(4-(benzyloxy)-5-methyl-1H-indol-3-yl)-N-isopropyl-N-methyl-2-oxoacetamide (690 mg) as a brown solid which was used in the subsequent step without further purification. LCMS (ESI+): m/z 365.3 [M+H]⁺.

### Step 2: N-(2-(4-(benzyloxy)-5-methyl-1H-indol-3-yl)ethyl)-N-methylpropan-2-amine (114)

To an ice-cold solution of 2-(4-(benzyloxy)-5-methyl-1H-indol-3-yl)-N-isopropyl-N-methyl-2-oxoacetamide (600 mg) in anhydrous THF (20 mL) was added 1 M LiAlH₄, in THF (6.59 mL, 6.59 mmol). The reaction was stirred at 0° C. for 30 min, and then at reflux for 16 h. The reaction mixture was cooled to 0° C. and quenched with saturated aq. Na₂SO₄ solution (3 mL), filtered, and the filtrate concentrated. The residue was diluted with CH₂Cl₂ (25 mL), silica gel (100 mg) was added, and the resulting mixture stirred for 30 min. The resulting slurry was filtered through a celite pad, and the filter cake was washed with MeOH (20 mL). The combined filtrate was concentrated under reduced pressure to afford N-(2-(4-(benzyloxy)-5-methyl-1H-indol-3-yl)ethyl)-N-methylpropan-2-amine (400 mg) as a purple solid which was used in the subsequent step without further purification. LCMS (ESI+): m/z 337.3 [M+H]⁺.

### Step 3: 3-(2-(isopropyl(methyl)amino)ethyl)-5-methyl-1H-indol-4-ol (P-66)

A stirred solution of N-(2-(4-(benzyloxy)-5-methyl-1H-indol-3-yl)ethyl)-N-methylpropan-2-amine (400 mg) in degassed MeOH (15 mL) was treated with formic acid (0.3 mL) followed by Pd/C (120 mg, 10% w/w). The reaction was then stirred under H₂ gas at ambient pressure and temperature for 16 h. The reaction mixture was filtered through a celite pad and washed with MeOH (20 mL). The combined filtrate was concentrated under reduced pressure and the resultant residue was purified by reverse phase column chromatography (C18, 0.01% v/v HCO₂H in MeCN), followed by lyophilization, to afford 3-(2-(isopropyl(methyl)amino)ethyl)-5-methyl-1H-indol-4-ol (53 mg, 13% over 3 steps) as a brown solid. ¹H NMR (400 MHZ, DMSO-d₆): δ 10.45 (br s, 1H), 8.18 (s, 1H), 6.89 (s, 1H), 6.73 (d, J=8.0 Hz, 1H), 6.66 (d, J=8.0 Hz, 1H), 2.84-2.82 (m, 3H), 2.67-2.66 (m, 2H), 2.29 (s, 3H), 2.16 (s, 3H), 0.92 (d, J=6.8 Hz, 6H). LCMS (ESI+): m/z 247.2 [M+H]⁺. HPLC Purity (PDA): 98.9%

### Example 38: 3-(2-(diethylamino)ethyl)-5-methyl-1H-indol-4-ol (P-67)

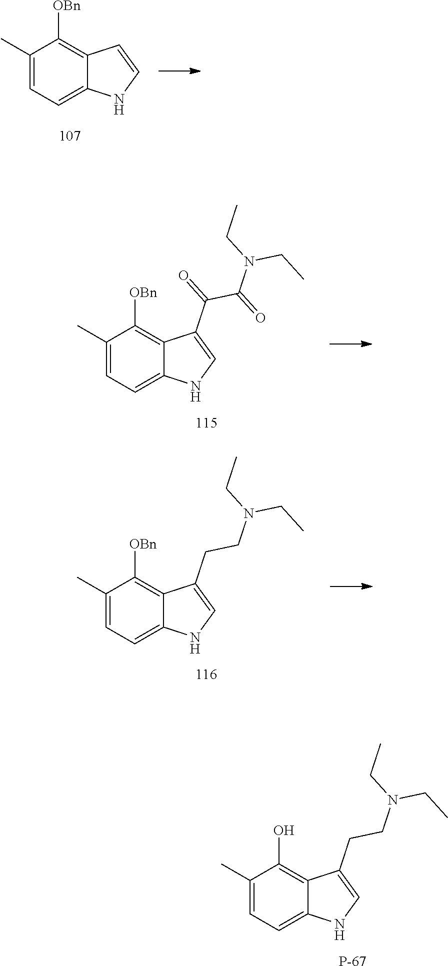

### Step 1: 2-(4-(benzyloxy)-5-methyl-1H-indol-3-yl)-N,N-diethyl-2-oxoacetamide (115)

To an ice-cold solution of 4-(benzyloxy)-5-methyl-1H-indole (400 mg, 1.69 mmol) in anhydrous THF (5 mL) was added oxalyl chloride (0.43 mL, 5.04 mmol). The reaction mixture was then stirred at ambient temperature for 2 h. The reaction was cooled again and diethylamine was added dropwise until the pH of the reaction solution was basic, at which point the reaction was stirred for 30 min before being quenched with H₂O (25 mL) and extracted with EtOAc (2×50 mL). The combined organic layers were washed with brine (20 mL), and dried over Na₂SO₄, filtered, and the filtrate concentrated under reduced pressure. The residue was purified by column chromatography (SiO₂, hexane: EtOAc, 6:4 to 5:5, v/v) to afford 2-(4-(benzyloxy)-5-methyl-1H-indol-3-yl)-N,N-diethyl-2-oxoacetamide (650 mg) as a brown solid which was used in the subsequent step without further purification. LCMS (ESI+): m/z 365.3 [M+H]⁺.

### Step 2: 2-(4-(benzyloxy)-5-methyl-1H-indol-3-yl)-N,N-diethylethan-1-amine (116)

To an ice-cold solution of 2-(4-(benzyloxy)-5-methyl-1H-indol-3-yl)-N,N-diethyl-2-oxoacetamide (600 mg) in anhydrous THF (20 mL) was added 1 M LiAlH₄, in THF (6.59 mL, 6.59 mmol). The reaction was stirred at 0° C. for 30 min, and then at reflux for 16 h. The reaction mixture was cooled to 0° C. and quenched with saturated aq. Na₂SO₄ solution (3 mL), filtered, and the filtrate concentrated. The residue was diluted with CH₂Cl₂ (25 mL), silica gel (100 mg) was added, and the resulting mixture stirred for 30 min. The resulting slurry was filtered through a celite pad and the filter cake was washed with MeOH (20 mL). The combined filtrate was concentrated under reduced pressure to afford 2-(4-(benzyloxy)-5-methyl-1H-indol-3-yl)-N,N-diethylethan-1-amine (420 mg) as a brown oil which was used in the subsequent step without further purification. LCMS (ESI+): m/z 337.7 [M+H]⁺.

### Step 3: 3-(2-(diethylamino)ethyl)-5-methyl-1H-indol-4-ol (P-67)

A stirred solution of 2-(4-(benzyloxy)-5-methyl-1H-indol-3-yl)-N,N-diethylethan-1-amine (400 mg) in degassed MeOH (15 mL) was treated with formic acid (0.3 mL) followed by Pd/C (120 mg, 10% w/w). The reaction was then stirred under H₂ gas at ambient pressure and temperature for 16 h. The reaction mixture was filtered through a celite pad and washed with MeOH (20 mL). The combined filtrate was concentrated under reduced pressure and the resultant residue purified by reverse phase column chromatography (C18, 0.01% v/v HCO₂H in MeCN), followed by lyophilization, to afford 3-(2-(diethylamino)ethyl)-5-methyl-1H-indol-4-ol (56 mg, 13% over 3 steps) as a brown solid. ¹H NMR (400 MHZ, DMSO-d₆): δ 10.48 (br s, 1H), 8.20 (s, 1H), 6.90 (s, 1H), 6.73 (d, J=8.0 Hz, 1H), 6.66 (d, J=8.0 Hz, 1H), 2.87 (t, J=5.2 Hz, 2H), 2.74 (t, J=5.6 Hz, 2H), 2.62 (q, J=7.2 Hz, 4H), 2.17 (s, 3H), 0.98 (t, J=7.2 Hz, 6H). LCMS (ESI+): m/z 247.2 [M+H]⁺. HPLC Purity (PDA): 97.9%

### Example 39: 3-(2-(dipropylamino)ethyl)-5-methyl-1H-indol-4-ol (P-68)

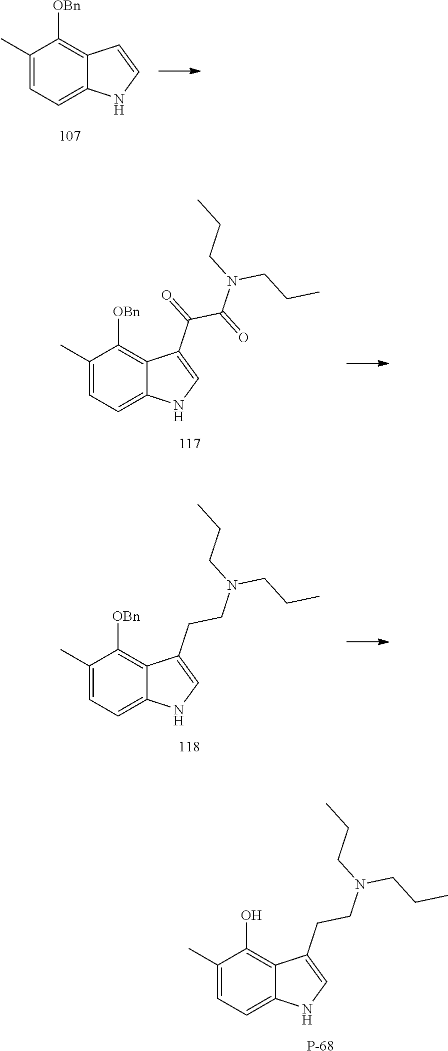

### Step 1: 2-(4-(benzyloxy)-5-methyl-1H-indol-3-yl)-N,N-dipropyl-2-oxoacetamide (117)

To an ice-cold solution of 4-(benzyloxy)-5-methyl-1H-indole (400 mg, 1.69 mmol) in anhydrous THF (5 mL) was added oxalyl chloride (0.43 mL, 5.04 mmol). The reaction mixture was then stirred at ambient temperature for 2 h. The reaction was cooled again and dipropylamine was added dropwise until the pH of the reaction solution was basic, at which point the reaction was stirred for 30 min before being quenched with H₂O (25 mL) and extracted with EtOAc (2×50 mL). The combined organic layers were washed with brine (20 mL), and dried over Na₂SO₄, filtered, and the filtrate concentrated under reduced pressure. The residue was purified by column chromatography (SiO₂, hexane: EtOAc, 6:4 to 5:5, v/v) to afford 2-(4-(benzyloxy)-5-methyl-1H-indol-3-yl)-N,N-dipropyl-2-oxoacetamide (610 mg) as a brown solid which was used in the subsequent step without further purification. LCMS (ESI+): m/z 393.4 [M+H]⁺.

### Step 2: 2-(4-(benzyloxy)-5-methyl-1H-indol-3-yl)-N,N-dipropylethan-1-amine (118)

To an ice-cold solution of 2-(4-(benzyloxy)-5-methyl-1H-indol-3-yl)-N,N-dipropyl-2-oxoacetamide (600 mg) in anhydrous THF (20 mL) was added 1 M LiAlH₄, in THF (6.11 mL, 6.11 mmol). The reaction was stirred at 0° C. for 30 min, and then at reflux for 16 h. The reaction mixture was cooled to 0° C. and quenched with saturated aq. Na₂SO₄ solution (3 mL), filtered, and the filtrate concentrated. The residue was diluted with CH₂Cl₂ (25 mL), silica gel (100 mg) was added, and the resulting mixture stirred for 30 min. The resulting slurry was filtered through a celite pad, and the filter cake was washed with MeOH (20 mL). The combined filtrate was concentrated under reduced pressure to afford 2-(4-(benzyloxy)-5-methyl-1H-indol-3-yl)-N,N-dipropylethan-1-amine (405 mg) as a purple solid which was used in the subsequent step without further purification. LCMS (ESI+): m/z 365.3 [M+H]⁺.

### Step 3: 3-(2-(dipropylamino)ethyl)-5-methyl-1H-indol-4-ol (P-68)

A stirred solution of 2-(4-(benzyloxy)-5-methyl-1H-indol-3-yl)-N,N-dipropylethan-1-amine (400 mg) in degassed MeOH (15 mL) was treated with formic acid (0.3 mL) followed by Pd/C (120 mg, 10% w/w). The reaction was stirred under H₂ gas at ambient pressure and temperature for 16 h. The reaction mixture was then filtered through a celite pad and washed with MeOH (20 mL). The combined filtrate was concentrated under reduced pressure and the resultant residue was purified by reverse phase column chromatography (C18, 0.01% v/v HCO₂H in MeCN), followed by lyophilization, to afford 3-(2-(dipropylamino)ethyl)-5-methyl-1H-indol-4-ol (55 mg, 12% over 3 steps) as a brown solid. ¹H NMR (400 MHZ, DMSO-d₆): δ 10.47 (br s, 1H), 8.19 (s, 1H), 6.89 (d, J=2.0 Hz, 1H), 6.73 (d, J=8.4 Hz, 1H), 6.66 (d, J=7.6 Hz, 1H), 2.90-2.85 (m, 2H), 2.76-2.72 (m, 2H), 2.50-2.47 (m, 4H), 2.16 (s, 3H), 1.47-1.41 (m, 4H), 0.79 (t, J=7.2 Hz, 6H); LCMS (ESI+): m/z 275.5 [M+H]⁺. HPLC Purity (PDA): 96.8%

### Example 40: 3-(2-(diisopropylamino)ethyl)-5-methyl-1H-indol-4-ol (P-69)

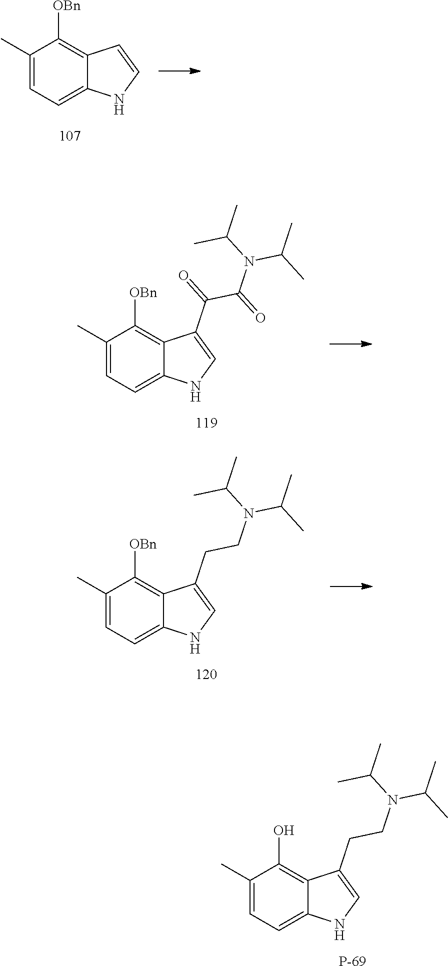

### Step 1: 2-(4-(benzyloxy)-5-methyl-1H-indol-3-yl)-N,N-diisopropyl-2-oxoacetamide (119)

To an ice-cold solution of 4-(benzyloxy)-5-methyl-1H-indole (400 mg, 1.69 mmol) in anhydrous THF (5 mL) was added oxalyl chloride (0.43 mL, 5.04 mmol). The reaction mixture was then stirred at ambient temperature for 2 h. The reaction was cooled again and diisopropylamine was added dropwise until the pH of the reaction solution was basic, at which point the reaction was stirred for 30 min before being quenched with H₂O (25 mL) and extracted with EtOAc (2×50 mL). The combined organic layers were washed with brine (20 mL), and dried over Na₂SO₄, filtered, and the filtrate concentrated under reduced pressure. The residue was purified by column chromatography (SiO₂, hexane: EtOAc, 6:4 to 5:5, v/v) to afford 2-(4-(benzyloxy)-5-methyl-1H-indol-3-yl)-N,N-diisopropyl-2-oxoacetamide (620 mg) as a white solid which was used in the subsequent step without further purification. LCMS (ESI+): m/z 393.3 [M+H]⁺.

### Step 2: N-(2-(4-(benzyloxy)-5-methyl-1H-indol-3-yl)ethyl)-N-isopropylpropan-2-amine (120)

To an ice-cold solution of 2-(4-(benzyloxy)-5-methyl-1H-indol-3-yl)-N,N-diisopropyl-2-oxoacetamide (600 mg) in anhydrous THF (20 mL) was added 1 M LiAlH₄, in THF (6.11 mL, 6.11 mmol). The reaction was stirred at 0° C. for 30 min, and then at reflux for 16 h. The reaction mixture was cooled to 0° C. and quenched with saturated aq. Na₂SO₄ solution (3 mL), filtered, and the filtrate concentrated. The residue was diluted with CH₂Cl₂ (25 mL), silica gel (100 mg) was added, and the resulting mixture stirred for 30 min. The resulting slurry was filtered through a celite pad, and the filter cake was washed with MeOH (20 mL). The combined filtrate was concentrated under reduced pressure to afford N-(2-(4-(benzyloxy)-5-methyl-1H-indol-3-yl)ethyl)-N-isopropylpropan-2-amine (420 mg) as a white solid which was used in the subsequent step without further purification. LCMS (ESI+): m/z 365.4 [M+H]⁺.

### Step 3: 3-(2-(diisopropylamino)ethyl)-5-methyl-1H-indol-4-ol (P-69)

A stirred solution of N-(2-(4-(benzyloxy)-5-methyl-1H-indol-3-yl)ethyl)-N-isopropylpropan-2-amine (400 mg) in degassed MeOH (15 mL) was treated with formic acid (0.3 mL) followed by Pd/C (120 mg, 10% w/w). The reaction was then stirred under H₂ gas at ambient pressure and temperature for 16 h. The reaction mixture was filtered through a celite pad and washed with MeOH (20 mL). The combined filtrate was concentrated under reduced pressure and the resultant residue purified by reverse phase column chromatography (C18, 0.01% v/v HCO₂H in MeCN), followed by lyophilization, to afford 3-(2-(diisopropylamino)ethyl)-5-methyl-1H-indol-4-ol (49 mg, 11% over 3 steps) as an off-white solid. ¹H NMR (400 MHZ, DMSO-d₆): δ 10.46 (br s, 1H), 8.21 (s, 1H), 6.90 (s, 1H), 6.73 (d, J=8.0 Hz, 1H), 6.66 (d, J=8.0 Hz, 1H), 3.10 (sept, J=6.4 Hz, 2H), 2.90-2.87 (m, 2H), 2.80-2.77 (m, 2H), 2.17 (s, 3H), 1.00 (d, J=6.4 Hz, 12H). LCMS (ESI+): m/z 275.3 [M+H]⁺. HPLC Purity (PDA): 95.2%.

Scheme 10: Compounds of general formula (I) can be synthesised from the appropriately substituted indole following the outlined sequence of steps in Scheme 10 or similar as one skilled in the art may consider. An appropriately substituted indole could be glyoxylated with oxalyl chloride followed by treatment with an appropriately substituted amine to give glyoxamide intermediates. Such intermediates could then be subjected to reductive conditions to provide access to compounds of general formula (I) (exemplified by P-11). Subsequent demethylation of such compounds allows access to compounds of general formula (I) (exemplified by P-12). One skilled in the art will recognise that utilising differentially substituted amines would allow access to compounds of general formula (I) disclosed herein.

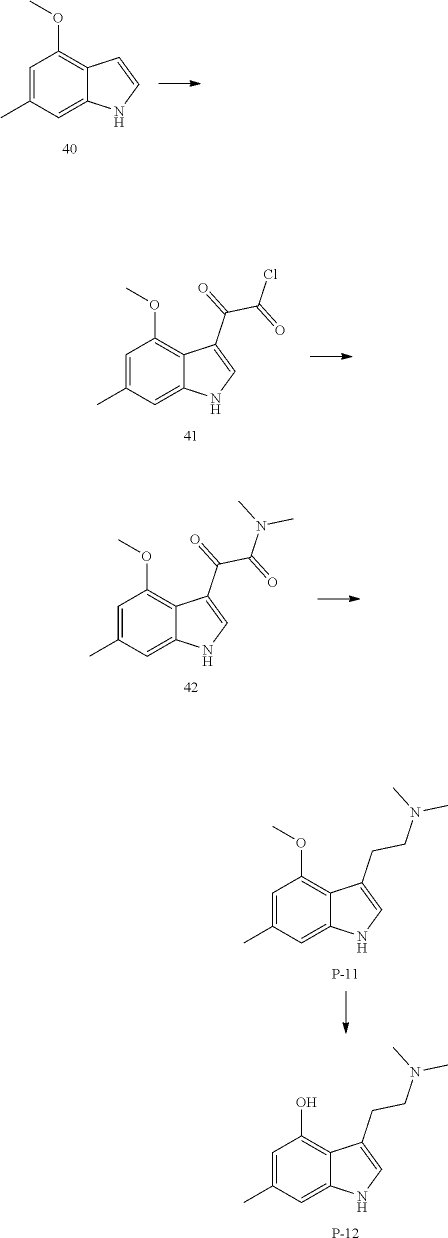

### Example 41: 3-(2-(dimethylamino)ethyl)-6-methyl-1H-indol-4-ol (P-12)

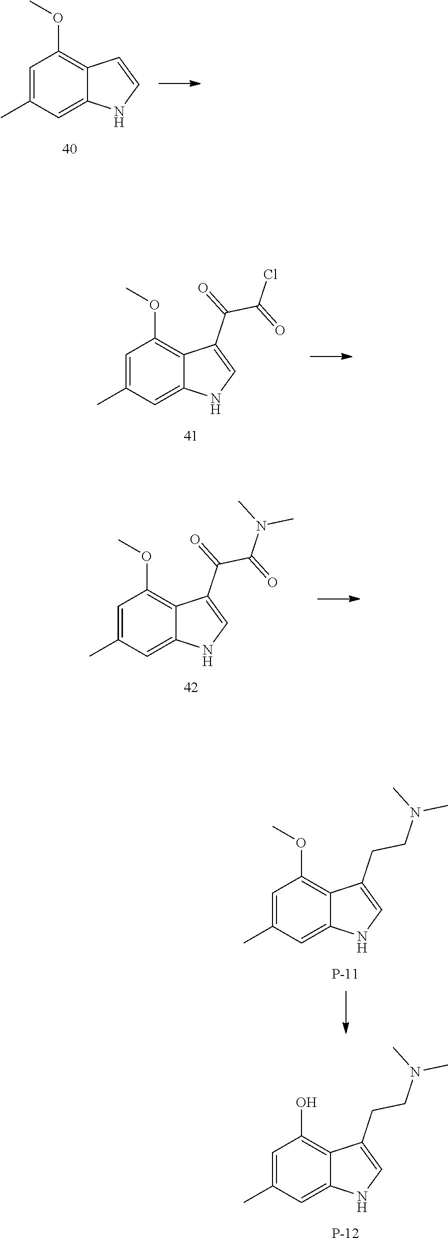

### Step 1: 2-(4-methoxy-6-methyl-1H-indol-3-yl)-2-oxoacetyl chloride (41)

To a solution of 4-methoxy-6-methyl-1H-indole (2.12 g, 13.2 mmol) in THF (20 mL) was added (COCl)₂ (2.51 g, 19.8 mmol) at 0° C. under N₂ gas. The reaction mixture was stirred at 0° C. for 2 h under N₂ gas. The solvent was evaporated to give 2-(4-methoxy-6-methyl-1H-indol-3-yl)-2-oxoacetyl chloride (3.31 g) as a brown solid which was used in the subsequent reactions without further purification.

### Step 2: 2-(4-methoxy-6-methyl-1H-indol-3-yl)-N,N-dimethyl-2-oxoacetamide (42)

A solution of crude 2-(4-methoxy-6-methyl-1H-indol-3-yl)-2-oxoacetyl chloride (3.31 g) dissolved in minimal anhydrous THF was added dropwise into Me₂NH (2.0 M in THF, 30 mL) at 0° C. under N₂. The reaction mixture was stirred at 25° C. for 12 h and the solvent was evaporated. The residue was purified by column chromatography (SiO₂, petroleum ether/EtOAc, v/v, 9:1 to 8:2) to give 2-(4-methoxy-6-methyl-1H-indol-3-yl)-N,N-dimethyl-2-oxoacetamide (2.00 g, 58% over 2 steps) as a brown solid. ¹H NMR (400 MHZ, MeOD-d₄): δ 7.98 (s, 1H), 6.90 (s, 1H), 6.59 (s, 1H), 3.89 (s, 3H), 3.10 (s, 3H), 3.04 (s, 3H), 2.43 (s, 3H).

### Step 3: 2-(4-methoxy-6-methyl-1H-indol-3-yl)-N,N-dimethylethan-1-amine (P-11)

To a solution of 2-(4-methoxy-6-methyl-1H-indol-3-yl)-N,N-dimethyl-2-oxoacetamide (300 mg, 1.15 mmol) in THF (10 mL) was added LiAlH₄ (525 mg, 13.8 mmol). The reaction mixture was stirred at 70° C. for 3 h, cooled to 0° C., and quenched by portionwise addition of Na₂SO₄·10H₂O (2.0 g). The mixture was filtered, the filter cake was washed with THF (50 mL), and the filtrate evaporated. The crude product was purified by preparative HPLC (column: Waters Xbridge Prep OBD (C18 150×40 mm×10 μm; mobile phase: [water (NH₄HCO₃)-ACN]; B: 15-45%, 8 min) to give 2-(4-methoxy-6-methyl-1H-indol-3-yl)-N,N-dimethylethan-1-amine (172 mg, 64%) as an off-white solid. ¹H NMR (400 MHZ, MeOD-d₄): δ 6.79 (s, 1H), 6.72 (s, 1H), 6.28 (s, 1H), 3.87 (s, 3H), 3.00-2.96 (m, 2H), 2.64-2.60 (m, 2H), 2.38 (s, 3H), 2.33 (s, 6H). LCMS (ESI+): m/z 233.3 [M+H]⁺; HPLC Purity (254 nm): 95%.

### Step 4: 3-(2-(dimethylamino)ethyl)-6-methyl-1H-indol-4-ol (P-12)

To a solution of 2-(4-methoxy-6-methyl-1H-indol-3-yl)-N,N-dimethylethan-1-amine (500 mg, 2.15 mmol) in CH₂Cl₂ (10 mL) was added AlCl₃ (1.72 g, 12.9 mmol) and EtSH (2.41 g, 38.7 mmol) at 0° C. under N₂ gas. The reaction mixture was stirred at 25° C. for 5 h, and then quenched by addition of saturated aqueous NaHCO₃ solution (10 mL). The aqueous phase was extracted with CH₂Cl₂ (10 mL×3) and the combined organic layers were dried over anhydrous Na₂SO₄, filtered, and the filtrate concentrated. The residue was purified by preparative HPLC (column: Phenomenex Luna 80×30 mm×3 μm; mobile phase: [water (TFA)-ACN]; B: 1-25%, 8 min) to give 3-(2-(dimethylamino)ethyl)-6-methyl-1H-indol-4-ol as the trifluoroacetate salt (20.6 mg, 3%) which was an off-white solid. ¹H NMR: (400 MHZ, MeOD-d₄): δ 6.95 (s, 1H), 6.66 (s, 1H), 6.23 (s, 1H), 3.51 (t, J=7.6 Hz, 2H), 3.25 (t, J=7.6 Hz, 2H), 2.90 (s, 6H), 2.32 (s, 3H). LCMS (ESI+): m/z 219.2 [M+H]⁺. HPLC Purity (220 nm): 98.0%

### Example 42: N-ethyl-2-(4-methoxy-6-methyl-1H-indol-3-yl)-N-methylethan-1-amine (P-70)

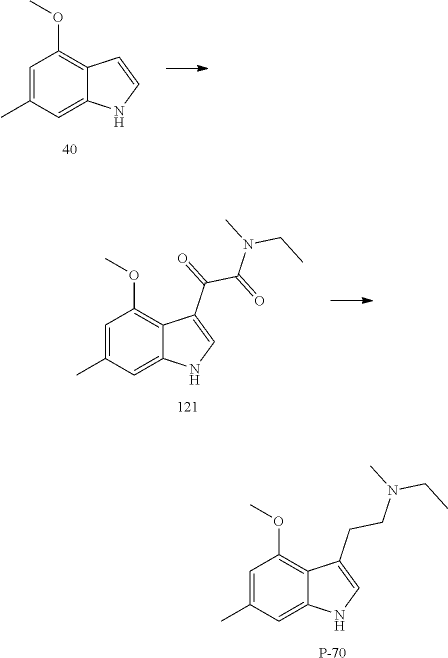

### Step 1: N-ethyl-2-(4-methoxy-6-methyl-1H-indol-3-yl)-N-methyl-2-oxoacetamide (121)

To an ice-cold stirred solution of 4-methoxy-6-methyl-1H-indole (0.5 g, 3.1 mmol) in anhydrous Et₂O (100 mL) was added dropwise a 1:1 solution of anhydrous Et₂O and oxalyl chloride (0.53 mL, 6.2 mmol), under N₂. Stirring was continued for 30 min at which time a solution of methylethylamine in anhydrous Et₂O (1:1, v/v), was added over 1 h until the pH of the reaction solution was basic. The reaction was stirred at ambient temperature for 1 h and then concentrated under a flow of N₂ gas. The residue was taken up in EtOAc (100 mL) and washed with H₂O (20 mL×3), and then brine (20 mL). The organic layer was dried over anhydrous Na₂SO₄, filtered, and the filtrate concentrated in vacuo. The residue was purified by flash chromatography (SiO₂, 0.1% to 5% MeOH in CH₂Cl₂) to afford N-ethyl-2-(4-methoxy-6-methyl-1H-indol-3-yl)-N-methyl-2-oxoacetamide (800 mg, 94%) as a light brown oil. The product was a mixture of two rotamers in a ratio of approximately 1:2 (A:B).

Rotamer A: ¹H NMR (400 MHZ, MeOD-d₄): δ 7.97 (s, 1H), 6.90 (s, 1H), 6.63-6.57 (m, 1H), 3.88 (s, 3H), 3.39 (q, J=7.2 Hz, 2H), 3.07 (s, 3H), 2.43 (s, 3H), 1.18 (t, J=7.2 Hz, 3H). ¹³C NMR (101 MHZ, MeOD-d₄): δ 188.5, 170.8, 154.9, 140.9, 136.5, 136.3, 115.5, 113.6, 106.4, 106.2, 56.1, 46.1, 31.6, 21.9, 13.3. Rotamer B: ¹H NMR (400 MHZ, MeOD-d₄): δ 7.93 (s, 1H), 6.90 (s, 1H), 6.63-6.57 (m, 1H), 3.88 (s, 3H), 3.58 (q, J=7.2 Hz, 2H), 3.01 (s, 3H), 2.43 (s, 3H), 1.25 (t, J=7.2 Hz, 3H). ¹³C NMR (101 MHZ, MeOD-d₄): δ 188.5, 170.8, 154.9, 140.9, 136.5, 136.4, 115.5, 113.6, 106.4, 106.3, 54.8, 42.6, 35.3, 21.9, 12.2.

### Step 2: N-ethyl-2-(4-methoxy-6-methyl-1H-indol-3-yl)-N-methylethan-1-amine (P-70)

To an ice-cold solution of anhydrous THF (15 mL) was added LiAlH₄ (398 mg, 8 eq., 10.5 mmol) in portions. The resulting ice-cold suspension was then treated dropwise with N-ethyl-2-(4-methoxy-6-methyl-1H-indol-3-yl)-N-methyl-2-oxoacetamide (360 mg, 1.31 mmol), pre-dissolved in minimal anhydrous THF, at a rate which maintained a gentle reflux. The reaction mixture was then refluxed under N₂ for 3 h before being cooled in an ice bath and quenched by sequential dropwise addition of cold H₂O (0.4 mL), 3.75 M aq. NaOH (0.4 mL), and H₂O (1.2 mL). The resulting suspension was then dried with Na₂SO₄ and filtered through a pad of celite. The filter cake was eluted with additional hot THF (50 mL×2) and the combined filtrate was concentrated in vacuo to afford N-ethyl-2-(4-methoxy-6-methyl-1H-indol-3-yl)-N-methylethan-1-amine (291 mg, 90%) as a lightly coloured oil. ¹H NMR (400 MHZ, MeOD-d₄): δ 6.78 (s, 1H), 6.72 (s, 1H), 6.27 (s, 1H), 3.85 (s, 3H), 3.03-2.89 (m, 2H), 2.71-2.63 (m, 2H), 2.53 (q, J=7.2 Hz, 2H), 2.37 (s, 3H), 2.32 (s, 3H), 1.10 (t, J=7.2 Hz, 3H). ¹³C NMR (101 MHz, MeOD-d₄): δ 155.4, 140.2, 133.1, 121.3, 116.4, 113.9, 105.6, 101.6, 60.2, 55.2, 52.0, 41.8, 24.9, 22.1, 11.9.

### Step 2a: N-ethyl-2-(4-methoxy-6-methyl-1H-indol-3-yl)-N-methylethan-1-amine fumarate (P-70-fumarate)

To a solution of fumaric acid (118 mg, 1.01 mmol) in minimal refluxing acetone was added a solution of N-ethyl-2-(4-methoxy-6-methyl-1H-indol-3-yl)-N-methylethan-1-amine (250 mg, 1.01 mmol) in minimal warm acetone. The resulting solution was allowed to cool to ambient temperature and stood overnight to afford the title compound as the fumarate salt (284 mg, 77%) which were colourless crystals. 1H NMR (400 MHZ, DMSO-d₆): δ 10.67 (s, 1H), 6.93 (d, J=2.2 Hz, 1H), 6.72 (s, 1H), 6.51 (s, 2H), 6.28 (s, 1H), 3.83 (s, 3H), 3.04-2.88 (m, 4H), 2.84 (d, J=7.2 Hz, 2H), 2.54 (s, 3H), 2.34 (s, 3H), 1.14 (t, J=7.2 Hz, 3H). ¹³C NMR (100 MHZ, DMSO-d₆): δ 167.6, 153.4, 138.3, 134.9, 131.3, 121.3, 114.5, 109.9, 104.7, 100.7, 56.1, 54.8, 49.6, 39.8, 21.8, 21.7, 9.4. qNMR Purity (ERETIC): 100%.

### Example 43: N-(2-(4-methoxy-6-methyl-1H-indol-3-yl)ethyl)-N-methylpropan-2-amine (P-71)

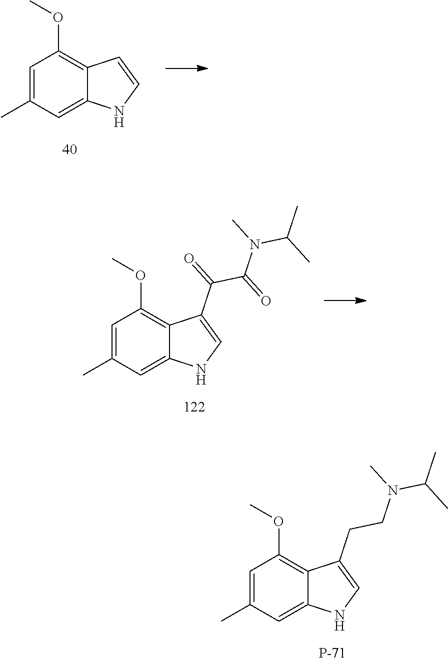

### Step 1: N-isopropyl-2-(4-methoxy-6-methyl-1H-indol-3-yl)-N-methyl-2-oxoacetamide (122)

To an ice-cold solution of 4-methoxy-6-methyl-1H-indole (0.5 g, 3.1 mmol) in anhydrous Et₂O (100 mL) was added dropwise a 1:1 solution of anhydrous Et₂O and oxalyl chloride (0.53 mL, 2 eq., 6.20 mmol), under N₂ and stirring maintained for an additional 30 min. The reaction was then treated with solution of Methyl(isopropyl)amine in anhydrous Et₂O (1:1, v/v) over 1 h until the pH of the reaction solution was basic and stirring maintained at ambient temperature for 1 h. The solution was then concentrated under a flow of N₂ gas and the residue suspended in H₂O (20 mL) and extracted with EtOAc (50 mL×3). The combined organic layers were washed with brine (50 mL), dried over anhydrous Na₂SO₄, filtered, and the filtrate concentrated in vacuo. The residue was purified by flash chromatography (SiO₂, 0.1% to 5% MeOH in CH₂Cl₂) to afford N-isopropyl-2-(4-methoxy-6-methyl-1H-indol-3-yl)-N-methyl-2-oxoacetamide (800 mg, 89%) as a light brown oil. The product was a mixture of two rotamers in a ratio of approximately 1:3 (A:B). ¹H NMR (400 MHZ, MeOD-d₄): δ 8.00 (s, 0.75H), 7.89 (s, 0.25H), 6.90 (s, 1H), 6.60 (m, 1H), 4.78 (sept, J=6.8 Hz, 0.25H), 4.01 (sept, J=6.8 Hz, 0.75H), 3.88 (m, 3H), 2.96 (s, 2.25H), 2.87 (s, 0.75H), 2.43 (s, 3H), 1.26 (d, J=6.8 Hz, 1.5H), 1.20 (d, J=6.8 Hz, 4.5H) [mixture of 2 rotamers]. ¹³C NMR (101 MHZ, MeOD-d₄; major rotamer) δ 187.0, 169.4, 153.5, 139.5, 135.5, 135.1, 114.1, 112.1, 105.1, 104.9, 54.6, 49.7, 24.1, 20.5, 18.5.

### Step 2: N-(2-(4-methoxy-6-methyl-1H-indol-3-yl)ethyl)-N-methylpropan-2-amine (P-71)

To ice-cold anhydrous THF (30 mL) was added LiAlH₄ (621 mg, 16.4 mmol) in portions. The resulting ice-cold suspension was then treated dropwise with N-isopropyl-2-(4-methoxy-6-methyl-1H-indol-3-yl)-N-methyl-2-oxoacetamide (590 mg, 2.05 mmol) pre-dissolved in minimal anhydrous THF, at a rate which maintained a gentle reflux. The reaction mixture was then refluxed under N₂ for 3 h before being cooled in an ice bath and quenched by sequential dropwise addition of cold H₂O (0.6 mL), 3.75 M aq. NaOH (0.6 mL), and H₂O (1.8 mL). The resulting suspension was then dried with Na₂SO₄ and filtered through a pad of celite. The filter cake was eluted with additional hot THF (50 mL×2) and the combined filtrate was concentrated in vacuo to afford N-(2-(4-methoxy-6-methyl-1H-indol-3-yl)ethyl)-N-methylpropan-2-amine (396 mg, 74%) as a lightly coloured oil. ¹H NMR (400 MHZ, MeOD-d₄): δ 6.79 (s, 1H), 6.72 (s, 1H), 6.28 (s, 1H), 3.86 (s, 3H), 3.01-2.85 (m, 3H), 2.75-2.65 (m, 2H), 2.38 (s, 3H), 2.33 (s, 3H), 1.07 (d, J=6.8 Hz, 6H). ¹³C NMR (101 MHZ, MeOD-d₄): δ 155.3, 140.2, 133.1, 121.3, 116.4, 114.0, 105.6, 101.5, 56.8, 55.2, 54.6, 37.8, 25.8, 22.1, 18.1.

### Step 2a: N-(2-(4-methoxy-6-methyl-1H-indol-3-yl)ethyl)-N-methylpropan-2-amine fumarate (P-71-fumarate)

To a solution of fumaric acid (134 mg, 1.15 mmol) in minimal refluxing acetone was added a solution of N-(2-(4-methoxy-6-methyl-1H-indol-3-yl)ethyl)-N-methylpropan-2-amine (0.3 g, 1.15 mmol) in minimal warm acetone. The resulting solution was allowed to cool to ambient temperature and stood overnight to afford N-(2-(4-methoxy-6-methyl-1H-indol-3-yl)ethyl)-N-methylpropan-2-amine as the fumarate salt (398 mg, 92%) which were yellow crystals. ¹H NMR (400 MHZ, DMSO-d₆): δ 10.76-10.68 (m, 1H), 6.94 (d, J=2.4 Hz, 1H), 6.72 (s, 1H), 6.51 (s, 2H), 6.29 (s, 1H), 3.83 (s, 3H), 3.37 (sept, J=6.4 Hz, 1H), 3.02 (m, 4H), 2.57 (s, 3H), 2.34 (s, 3H), 1.17 (d, J=6.4 Hz, 6H); ¹³C NMR (100 MHZ, DMSO-d₆): δ 167.8, 153.4, 138.3, 135.1, 131.3, 121.3, 114.6, 110.1, 104.7, 100.7, 54.8, 54.5, 53.7, 35.5, 22.7, 21.7, 16.4; qNMR Purity (ERETIC): 99.2%.

### Example 44: N, N-diethyl-2-(4-methoxy-6-methyl-1H-indol-3-yl)ethan-1-amine (P-72)

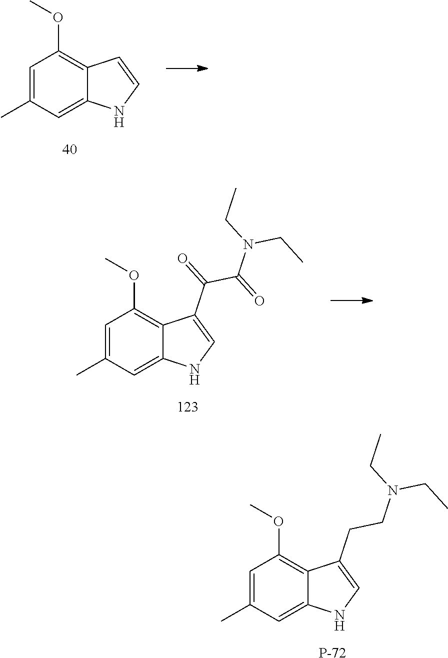

### Step 1: N,N-diethyl-2-(4-methoxy-6-methyl-1H-indol-3-yl)-2-oxoacetamide (123)

To an ice-cold solution of 4-methoxy-6-methyl-1H-indole (0.5 g, 3.1 mmol) in anhydrous Et₂O (50 mL) was added dropwise a 1:1 solution of anhydrous Et₂O and oxalyl chloride (0.40 mL, 1.5 eq., 4.65 mmol) under N₂ and stirring continued for 30 min at which point a solution of diethylamine (1.93 mL, 6 eq., 18.6 mmol) in Et₂₀ (2 mL) was added dropwise and stirring maintained for 2 h. The reaction was then concentrated under a flow of N₂ gas, and the residue was taken up in EtOAc (100 mL) and subsequently washed with 0.5 M aq. HCl (20 mL×3), H₂O (20 mL), then brine (50 mL). The organic layer was dried over anhydrous Na₂SO₄, filtered, and the filtrate concentrated in vacuo. The resulting residue was purified by flash chromatography (SiO₂, 0.1% to 5% MeOH in CH₂Cl₂) to afford N,N-diethyl-2-(4-methoxy-6-methyl-1H-indol-3-yl)-2-oxoacetamide (726 mg, 81%) as a light brown oil. ¹H NMR (400 MHZ, MeOD-d₄): δ 7.92 (s, 1H), 6.90 (s, 1H), 6.59 (s, 1H), 3.88 (s, 3H), 3.56 (q, J=7.2 Hz, 2H), 3.40 (q, J=7.2 Hz, 2H), 2.43 (s, 3H), 1.27 (t, J=7.2 Hz, 3H), 1.17 (t, J=7.2 Hz, 3H). ¹³C NMR (101 MHZ, MeOD-d₄): δ 188.2, 170.8, 155.0, 140.9, 136.6, 136.5, 115.5, 113.6, 106.4, 106.3, 56.0, 44.0, 40.3, 21.9, 14.2, 13.2.

### Step 2: N,N-diethyl-2-(4-methoxy-6-methyl-1H-indol-3-yl)ethan-1-amine (P-72)

To ice-cold anhydrous THF (50 mL) was added LiAlH₄ (726 mg, 8 eq., 19.1 mmol) in portions and the resulting ice-cold suspension was treated dropwise with N,N-diethyl-2-(4-methoxy-6-methyl-1H-indol-3-yl)-2-oxoacetamide (690 mg, 2.39 mmol), pre-dissolved in minimal anhydrous THF, at a rate which maintained a gentle reflux. The reaction mixture was refluxed under N₂ for 3 h before being cooled in an ice bath and quenched by sequential dropwise addition of cold H₂O (0.8 mL), 3.75 M aq. NaOH (0.8 mL), and H₂O (2.4 mL). The resulting suspension was then dried with Na₂SO₄ and filtered through a pad of celite. The filter cake was eluted with additional hot THF (100 mL×2) and the combined filtrate was concentrated in vacuo to afford N,N-diethyl-2-(4-methoxy-6-methyl-1H-indol-3-yl)ethan-1-amine (620 mg, quant.) as a slightly coloured oil. ¹H NMR (400 MHZ, MeOD-d₄): δ 6.79 (s, 1H), 6.73 (s, 1H), 6.29 (s, 1H), 3.87 (s, 3H), 3.01-2.87 (m, 2H), 2.80-2.72 (m, 2H), 2.67 (q, J=7.2 Hz, 4H), 2.38 (s, 3H), 1.13 (t, J=7.2 Hz, 6H). ¹³C NMR (100 MHZ, MeOD-d₄): δ 155.3, 140.2, 133.1, 121.3, 116.4, 114.1, 105.6, 101.5, 55.9, 55.2, 47.9, 24.3, 22.1, 11.5.

### Step 2a: N,N-diethyl-2-(4-methoxy-6-methyl-1H-indol-3-yl)ethan-1-amine fumarate (P-72⋅fumarate)

To a solution of fumaric acid (125 mg, 1.08 mmol) in minimal refluxing acetone was added a solution of N, N-diethyl-2-(4-methoxy-6-methyl-1H-indol-3-yl)ethan-1-amine (280 mg, 1.08 mmol) in minimal warm acetone. The resulting solution was allowed to cool to ambient temperature and stood overnight to afford N,N-diethyl-2-(4-methoxy-6-methyl-1H-indol-3-yl)ethan-1-amine as the fumarate salt (247 mg, 52%) which were colourless crystals. ¹H NMR (400 MHZ, DMSO-d₆): δ 10.66 (s, 1H), 6.93 (d, J=2.2 Hz, 1H), 6.72 (s, 1H), 6.48 (s, 2H), 6.28 (s, 1H), 3.83 (s, 3H), 3.00-2.79 (m, 8H), 2.34 (s, 3H), 1.12 (t, J=7.2 Hz, 6H). ¹³C NMR (100 MHz, DMSO-d₆): δ 167.3, 153.3, 138.3, 134.8, 131.3, 121.4, 114.5, 110.0, 104.7, 100.7, 54.8, 52.5, 46.1, 21.7, 21.6, 9.4. qNMR Purity (ERETIC): 100%.

### Example 45: N-isopropyl-N-(2-(4-methoxy-6-methyl-1H-indol-3-yl)ethyl)propan-2-amine (P-73)

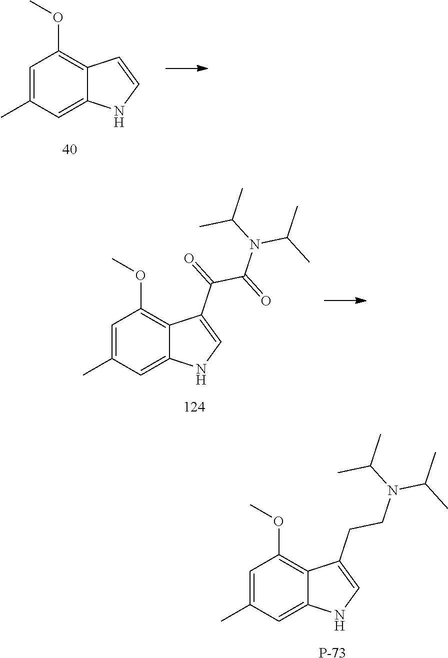

### Step 1: N,N-diisopropyl-2-(4-methoxy-6-methyl-1H-indol-3-yl)-2-oxoacetamide (124)

An ice-cold solution of 4-methoxy-6-methyl-1H-indole (0.5 g, 3.1 mmol) in anhydrous Et₂O (100 mL) was treated with a solution of oxalyl chloride (0.53 mL, 2 eq., 6.2 mmol) in Et₂O (0.5 mL) and stirring continued for a further 30 min. A solution of diisopropylamine in anhydrous Et₂O (1:1 v/v) was then added over 1 hour until the pH of the reaction solution was basic, which required a total amount of 15 equivalents (6.52 mL, 46.5 mmol). Stirring was continued for at ambient temperature for an additional 1 h before being concentrated under a flow of N₂ gas. The residue was taken up in CH₂Cl₂ (100 mL) and subsequently washed with H₂O (20 mL×3), and brine (50 mL) before being dried over anhydrous Na₂SO₄, filtered, and the filtrate concentrated. The resulting residue was purified by flash chromatography (SiO₂, 0.1% to 5% MeOH in CH₂Cl₂) to afford N, N-diisopropyl-2-(4-methoxy-6-methyl-1H-indol-3-yl)-2-oxoacetamide (800 mg, 82%) as a light brown oil. ¹H NMR (400 MHZ, MeOD-d₄): δ 7.83 (s, 1H), 6.91 (s, 1H), 6.62 (s, 1H), 3.98-3.85 (m, 4H), 3.66 (sept, J=6.8 Hz, 1H), 2.44 (s, 3H), 1.55 (d, J=6.8 Hz, 6H), 1.20 (d, J=6.8 Hz, 6H). ¹³C NMR (101 MHz, MeOD-d₄): δ 187.5, 170.7, 155.3, 141.2, 137.5, 136.8, 115.3, 110.4, 106.7, 106.4, 52.2, 47.1, 21.9, 20.6, 20.4.

### Step 2: N-isopropyl-N-(2-(4-methoxy-6-methyl-1H-indol-3-yl)ethyl)propan-2-amine (P-73)

To ice-cold anhydrous THF (20 mL) was added LiAlH₄ (480 mg, 8 eq., 12.6 mmol) in portions and the resulting ice-cold suspension was then treated dropwise with N,N-diisopropyl-2-(4-methoxy-6-methyl-1H-indol-3-yl)-2-oxoacetamide (0.5 g, 1.58 mmol), pre-dissolved in minimal anhydrous THF, at a rate which maintained a gentle reflux. The reaction mixture was refluxed under N₂ for a further 3 h before being cooled in an ice bath and quenched by sequential dropwise addition of cold H₂O (0.5 mL), 3.75 M aq. NaOH (0.5 mL), and H₂O (1.5 mL). The resulting suspension was then dried with Na₂SO₄ and filtered through a pad of celite. The filter cake was eluted with additional hot THF (50 mL×2) and the combined filtrate was concentrated in vacuo to afford N-isopropyl-N-(2-(4-methoxy-6-methyl-1H-indol-3-yl)ethyl)propan-2-amine (120 mg, 26%) as a colourless oil. ¹H NMR (400 MHZ, MeOD-d₄): δ 6.78 (s, 1H), 6.73 (s, 1H), 6.29 (s, 1H), 3.87 (s, 3H), 3.11 (sept, J=6.4 Hz, 2H), 2.95-2.86 (m, 2H), 2.75-2.61 (m, 2H), 2.38 (s, 3H), 1.13 (d, J=6.4 Hz, 12H).

### Step 2a: N-isopropyl-N-(2-(4-methoxy-6-methyl-1H-indol-3-yl)ethyl)propan-2-amine (P-73⋅fumarate)

To a solution of fumaric acid (33.8 mg, 0.29 mmol) in minimal refluxing acetone was added a solution of N-isopropyl-N-(2-(4-methoxy-6-methyl-1H-indol-3-yl)ethyl)propan-2-amine (84 mg, 0.29 mmol) in minimal warm acetone. The resulting solution was allowed to cool to ambient temperature and stood overnight to afford N-isopropyl-N-(2-(4-methoxy-6-methyl-1H-indol-3-yl)ethyl)propan-2-amine as the fumarate salt (108 mg, 92%) which were yellow crystals. ¹H NMR (400 MHZ, DMSO-d₆): δ 10.72-10.66 (m, 1H), 6.96 (d, J=2.0 Hz, 1H), 6.72 (s, 1H), 6.52 (s, 2H), 6.29 (s, 1H), 3.83 (s, 3H), 3.47-3.29 (m, 2H), 3.03-2.79 (m, 4H), 2.34 (s, 3H), 1.18 (d, J=6.4 Hz, 12H). ¹³C NMR (101 MHZ, DMSO-d₆): δ 167.1, 153.3, 138.3, 134.9, 131.1, 121.3, 114.6, 110.9, 104.7, 100.7, 54.8, 51.4, 48.3, 21.7, 19.6, 18.9. qNMR Purity (ERETIC): 98.6%.

Scheme 11: Compounds of general formula (I) can be synthesised from the appropriately substituted nitrobenzene following the outlined sequence of steps in Scheme 11 or similar as one skilled in the art may consider. Following standard deprotection conditions of methoxyarene 125 and subsequent benzylation of phenol 126 provided bromobenzene 127 that could undergo either a Grignard reaction or lithiation followed by alkylation to generate the tolyl intermediate 128. Subsequent Bartoli indole synthesis allowed access to substituted indole cores that could then undergo a similar sequence of synthetic transformations outlined in Scheme 6, namely, glyoxyamide formation with glyoxyl chloride and an appropriately substituted dialkyl amine. Subjecting the glyoxamide intermediates to reductive conditions provided access to tryptamines such as 132 and finally, hydrogenolysis of the benzyl protecting group gives access to compounds of general formula (I) (exemplified by P-74). One skilled in the art will recognise that utilising differentially substituted amines will allow access to a range of alkylated homologues of general formula (I) disclosed herein.

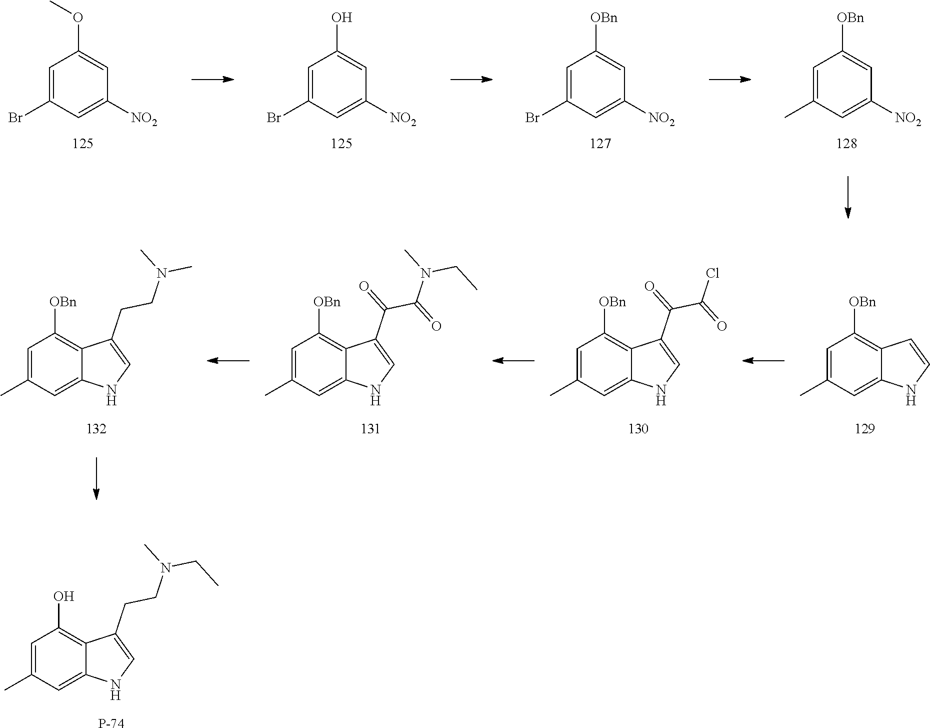

Scheme 12: Compounds of general formula (I) can be synthesised from the appropriately substituted nitrobenzene following the outlined sequence of steps in Scheme 12 or similar as one skilled in the art may consider. Application of the Bartoli indole synthesis allowed access to substituted indole cores that could then be condensed with dimethylamino-2-nitroethylene to give nitrovinyl indoles. Reduction of such indoles proved viable in accessing unsubstituted ethyl amine analogues that could subsequently undergo reductive alkylation to access compounds of general formula (I) (exemplified by P-13). One skilled in the art will recognise that protecting the amine with a suitable protecting group such as benzyl, followed by alkylation, subsequent deprotection, and a second alkylation would allow access to differentially alkylated compounds of general formula (I) disclosed herein.

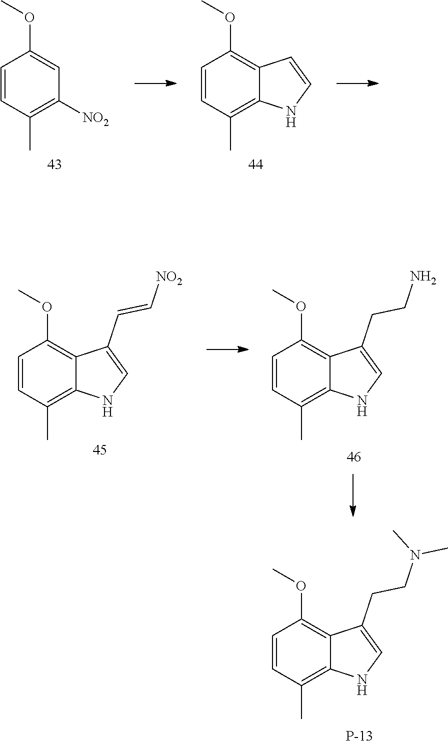

### Example 52: 2-(4-methoxy-7-methyl-1H-indol-3-yl)-N,N-dimethylethan-1-amine (P-13)

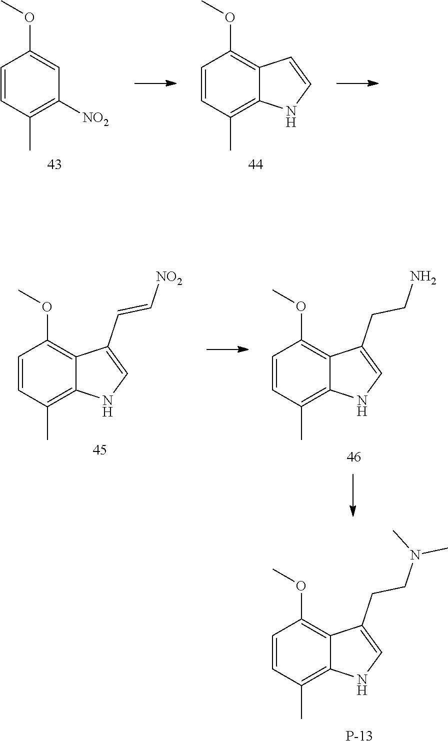

### Step 1: 4-methoxy-7-methyl-1H-indole (44)

To a solution of 4-methoxy-1-methyl-2-nitrobenzene (6.00 g, 35.8 mmol) in THF (42 mL) was added bromo (vinyl) magnesium (1.0 M, 179 mL, 179 mmol) at −65° C. The reaction mixture was stirred at −65° C. for 2 h. The mixture was poured into saturated aqueous NH₄Cl solution (1.5 L) and extracted with EtOAc (500 mL×2). The combined organic phase was washed with brine (80 mL), dried over anhydrous Na₂SO₄, filtered, and the filtrate concentrated in vacuo. The residue was purified by column chromatography (SiO₂, petroleum ether/EtOAc, v/v, 100:1 to 10:1) to give 4-methoxy-7-methyl-1H-indole (2.50 g, 39% yield) as a yellow solid. ¹H NMR (400 MHZ, CDCl₃): δ 8.08 (s, 1H), 7.14-7.15 (m, 1H), 6.91 (d, J=7.6 Hz, 1H), 6.68-6.70 (m, 1H), 6.47 (d, J=7.6 Hz, 1H), 3.96 (s, 3H), 2.45 (s, 3H).

### Step 2: (E)-4-methoxy-7-methyl-3-(2-nitrovinyl)-1H-indole (45)

To a solution of 4-methoxy-7-methyl-1H-indole (2.40 g, 14.8 mmol) in TFA (14.4 mL) was added N,N-dimethyl-2-nitroethen-1-amine (1.73 g, 14.8 mmol). The mixture was stirred at 20° C. for 0.5 h. The mixture was adjusted to pH 8 with saturated aqueous NaHCO₃ and extracted with EtOAc (50 mL×2). The combined organic phase was washed with brine (30 mL), dried over anhydrous Na₂SO₄, filtered, and concentrated in vacuo. The crude product was triturated with petroleum ether-EtOAc (5:1, 20 mL) at 20° C. for 30 min to give (E)-4-methoxy-7-methyl-3-(2-nitrovinyl)-1H-indole (3.30 g, 95% yield) as a red solid. LCMS (ESI+): m/z 233.1 [M+H]⁺; HPLC purity (220 nm): 95.7%.

### Step 3: 2-(4-methoxy-7-methyl-1H-indol-3-yl)ethan-1-amine (46)

To a solution of (E)-4-methoxy-7-methyl-3-(2-nitrovinyl)-1H-indole (2.60 g, 11.2 mmol) in THF (15 mL) was added LiAlH₄ (4.67 g, 123 mmol), and the mixture was stirred at 80° C. for 3 h. The mixture was cooled to 0° C., quenched by the addition of Na₂SO₄·10H₂O (10.0 g), stirred at 20° C. for 10 min, then filtered. The filtrate was concentrated in vacuo and the crude product was triturated with petroleum ether-EtOAc (5:1, 20 mL) at 20° C. for 30 min to give 2-(4-methoxy-7-methyl-1H-indol-3-yl)ethan-1-amine (1.30 g, 57% yield) as an off-white solid. ¹H NMR (400 MHZ, DMSO-d₆): δ 10.66 (s, 1H), 6.93 (s, 1H), 6.71 (d, J=7.6 Hz, 1H), 6.32 (d, J=7.6 Hz, 1H), 3.79 (s, 3H), 2.82-2.85 (m, 2H), 2.75-2.79 (m, 2H), 2.33 (s, 3H). LCMS (ESI+): m/z 205.2 [M+H]⁺.

### Step 4: 2-(4-methoxy-7-methyl-1H-indol-3-yl)-N,N-dimethylethan-1-amine (P-13)

To a solution of 2-(4-methoxy-7-methyl-1H-indol-3-yl)ethan-1-amine (500 mg, 2.45 mmol) in MeOH (5 mL) was added NaBH₃CN (307 mg, 4.90 mmol), AcOH (587 mg, 9.79 mmol) and 37% aqueous formaldehyde solution (496 mg, 6.12 mmol) at 0° C. The reaction mixture was stirred at 20° C. for 3 h. The mixture was adjusted to pH 8 with saturated aqueous Na₂CO₃ solution, then concentrated in vacuo. The residue was dissolved in H₂O (30 mL) and extracted with EtOAc (10 mL×2). The combined organic phase was washed with brine (10 mL), dried over anhydrous Na₂SO₄, filtered and the filtrate concentrated in vacuo. The residue was purified by preparative HPLC (column: Waters Xbridge Prep OBD C18 150*40 mm*10 μm; mobile phase: [water (NH₄HCO₃)-ACN]; B %: 5-40%, 8 min) to give 2-(4-methoxy-7-methyl-1H-indol-3-yl)-N,N-dimethylethan-1-amine (129 mg, 23% yield) as a yellow oil. ¹H NMR (400 MHZ, MeOD-d₄): δ 6.90 (s, 1H), 6.75 (d, J=7.6 Hz, 1H), 6.35 (d, J=7.6 Hz, 1H), 3.87 (s, 3H), 3.01-3.05 (m, 2H), 2.64-2.68 (m, 2H), 2.35-2.36 (m, 9H). LCMS (ESI+): m/z 233.2 [M+H]⁺. HPLC Purity (220 nm): 99.8%.

### Example 53: N-ethyl-2-(4-methoxy-7-methyl-1H-indol-3-yl)-N-methylethan-1-amine (P-80)

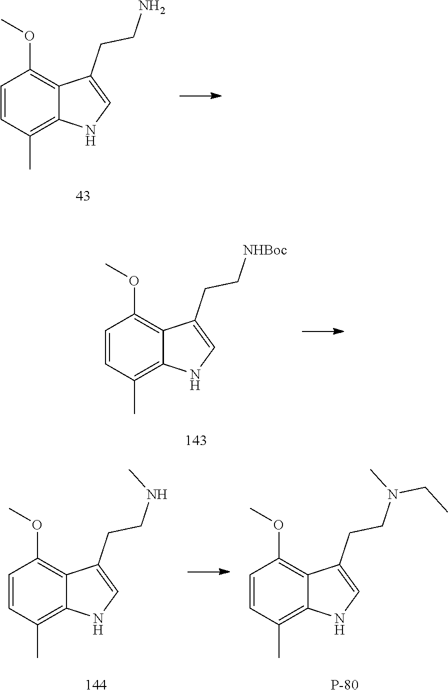

### Step 1: tert-butyl (2-(4-methoxy-7-methyl-1H-indol-3-yl)ethyl)carbamate (143)

A solution of 2-(4-methoxy-7-methyl-1H-indol-3-yl)ethan-1-amine (124 mg, 0.61 mmol) and Et₃N (65 mg, 0.64 mmol) in MeOH (3 mL) was degassed and purged with N₂ 3 times, and then treated with Boc₂O (140 mg, 0.64 mmol,) and stirred at 25° C. for 2 h under N₂. The mixture was concentrated in vacuo to give crude tert-butyl (2-(4-methoxy-7-methyl-1H-indol-3-yl)ethyl)carbamate (0.19 g) as a yellow solid which was used in the subsequent step without further purification.

### Step 2: 2-(4-methoxy-7-methyl-1H-indol-3-yl)-N-methylethan-1-amine (144)

An ice-cold (0° C.) solution of LiAlH₄ (2.50 M, 1.25 mL) in THF (3 mL) was treated with crude tert-butyl (2-(4-methoxy-7-methyl-1H-indol-3-yl)ethyl)carbamate (0.19 g) and stirred at 0° C. for 6 min before being heated to 70° C. for 3 h under a N₂ atmosphere. The reaction was then cooled to 0° C., quenched by the addition of Na₂SO₄·10H₂O (0.20 g), filtered, and the filtrate concentrated under reduced pressure. The crude product was triturated with EtOAc in petroleum ether (20:80, 10 mL) at 25° C. for 0.2 h to give 2-(4-methoxy-7-methyl-1H-indol-3-yl)-N-methylethan-1-amine (0.24 g) as a brown solid, which was used in the next step without further purification. LCMS (ESI+): m/z 219.2 [M+H]⁺.

### Step 3: N-ethyl-2-(4-methoxy-7-methyl-1H-indol-3-yl)-N-methylethan-1-amine (P-80)

To a solution of 2-(4-methoxy-7-methyl-1H-indol-3-yl)-N-methylethan-1-amine (120 mg, 0.55 mmol) in MeOH (3 mL) at 0° C. was added NaBH₃CN (51.8 mg, 0.83 mmol), 40% w/w aqueous acetaldehyde solution (121 mg, 1.10 mmol, 154 μL) then allowed to warm to 20° C. and stirring continued for 24 h. The mixture was concentrated in vacuo and the residue was purified by preparative HPLC (column: Waters Xbridge 150×25 mm×5 μm; mobile phase: [water (NH₄HCO₃)-ACN]; B: 18-48%, 9 min) to give N-ethyl-2-(4-methoxy-7-methyl-1H-indol-3-yl)-N-methylethan-1-amine (19.0 mg, 13%) as a white oil. ¹H NMR (400 MHZ, MeOD-d₄): δ 6.93 (s, 1H), 6.77 (d, J=7.6 Hz, 1H), 6.37 (d, J=7.6 Hz, 1H), 3.87 (s, 3H), 3.13-3.00 (m, 2H), 2.92-2.79 (m, 2H), 2.71 (q, J=7.2 Hz, 2H), 2.47 (s, 3H), 2.37 (s, 3H), 1.17 (t, J=7.2 Hz, 3H). LCMS (ESI+): m/z 247.3 [M+H]⁺. HPLC purity (220 nm): 95.3%.

### Example 54: N-(2-(4-methoxy-7-methyl-1H-indol-3-yl)ethyl)-N-methylpropan-2-amine (P-81)

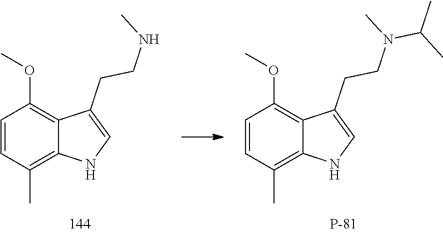

### Step 1: N-(2-(4-methoxy-7-methyl-1H-indol-3-yl)ethyl)-N-methylpropan-2-amine (P-81)

To a solution of 2-(4-methoxy-7-methyl-1H-indol-3-yl)-N-methylethan-1-amine (120 mg, 0.55 mmol) in MeOH (3 mL) at 0° C. was added NaBH₃CN (51.8 mg, 0.83 mmol), acetone (63.9 mg, 1.10 mmol, 80.8 L), and mixture was stirred at 20° C. for 24 h. The mixture was concentrated in vacuo and the residue was purified by preparative HPLC (column: Waters Xbridge 150×25 mm×5 μm; mobile phase: [water (NH₄HCO₃)-ACN]; B: 15-45%, 9 min) to give N-(2-(4-methoxy-7-methyl-1H-indol-3-yl)ethyl)-N-methylpropan-2-amine (28.0 mg, 20%) as a white oil. ¹H NMR (400 MHZ, MeOD-d₄): δ 6.95 (s, 1H), 6.79 (d, J=8.0 Hz, 1H), 6.39 (d, J=8.0 Hz, 1H), 3.89 (s, 3H), 3.11-3.02 (m, 3H), 2.88-2.82 (m, 2H), 2.46 (s, 3H), 2.39 (s, 3H), 1.15 (d, J=6.4 Hz, 6H); LCMS (ESI+): m/z 261.3 [M+H]⁺; HPLC purity (254 nm): 100%.

### Example 55: N, N-diethyl-2-(4-methoxy-7-methyl-1H-indol-3-yl)ethan-1-amine (P-82)

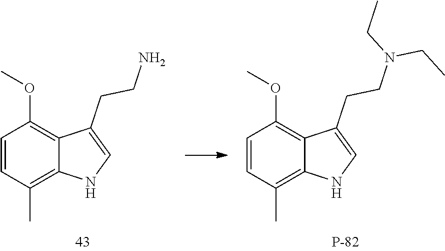

### Step 1: N,N-diethyl-2-(4-methoxy-7-methyl-1H-indol-3-yl)ethan-1-amine (P-82)

To a solution of 2-(4-methoxy-7-methyl-1H-indol-3-yl)-N-methylethan-1-amine (0.10 g, 0.49 mmol) in MeOH (3 mL) at 0° C. was added NaBH₃CN (61.5 mg, 0.98 mmol) and 40% w/w aqueous acetaldehyde solution (135 mg, 1.22 mmol, 172 μL), and the mixture was stirred at 20° C. for 3 h. The mixture was filtered and concentrated in vacuo, and the residue was suspended in H₂O (5 mL) and extracted with EtOAc (5 mL×2). The combined organic layers were washed with brine (30 mL), dried over anhydrous Na₂SO₄, filtered, and the filtrate concentrated in vacuo. The residue was purified by preparative HPLC (column: Waters Xbridge 150*25 mm*5 μm; mobile phase: [water (NH₄HCO₃)-ACN]; B %: 18%-48%, 9 min) to give N,N-diethyl-2-(4-methoxy-7-methyl-1H-indol-3-yl)ethan-1-amine (19.0 mg, 15%) as a brown oil. 1H NMR (400 MHZ MeOD-d₄): δ 6.95 (s, 1H), 6.77 (d, J=8.0 Hz, 1H), 6.38 (d, J=8.0 Hz, 1H), 3.88 (s, 3H), 3.14-2.94 (m, 4H), 2.87 (q, J=7.2 Hz, 4H), 2.37 (s, 3H), 1.20 (t, J=7.2 Hz, 6H). LCMS (ESI+): m/z 261.2. HPLC Purity (254 nm): 98.2%.

### Example 56: N-isopropyl-N-(2-(4-methoxy-7-methyl-1H-indol-3-yl)ethyl)propan-2-amine (P-83)

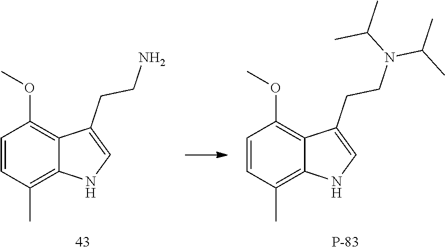

### Step 1: N-isopropyl-N-(2-(4-methoxy-7-methyl-1H-indol-3-yl)ethyl)propan-2-amine (P-83)

To a solution of 2-(4-methoxy-7-methyl-1H-indol-3-yl)-N-methylethan-1-amine (90.0 mg, 0.37 mmol) in MeOH (3 mL) at 0° C. was added NaBH₃CN (230 mg, 3.66 mmol) and acetone (425 mg, 7.31 mmol, 538 μL), and the mixture was stirred at 20° C. for 140 h. The mixture was filtered, and the filtrate concentrated in vacuo. The residue was suspended in H₂O (5 mL) and extracted with EtOAc (5 mL×2). The combined organic layers were washed with brine (30 mL), dried over anhydrous Na₂SO₄, filtered, and the filtrate concentrated in vacuo. The residue was purified by preparative HPLC (column: Waters Xbridge Prep OBD C18 150×40 mm×10 μm; mobile phase: [water (NH₄HCO₃)-ACN]; B: 25-55%, 15 min) to give N-isopropyl-N-(2-(4-methoxy-7-methyl-1H-indol-3-yl)ethyl)propan-2-amine (45 mg, 14%) as a brown oil. ¹H NMR (400 MHz, MeOD-d₄): δ 7.06 (s, 1H), 6.81 (d, J=7.6 Hz, 1H), 6.43 (d, J=7.6 Hz, 1H), 3.91 (s, 3H), 3.72-3.65 (m, 2H), 3.31-3.20 (m, 4H), 2.38 (s, 3H), 1.38 (d, J=6.4 Hz, 12H). LCMS (ESI+): m/z 289.2 [M+H]⁺. HPLC Purity (220 nm): 94.7%.

Scheme 13: Compounds of general formula (I) can be synthesised from the appropriately substituted nitrobenzene following the outlined sequence of steps in Scheme 13 or similar as one skilled in the art may consider. Application of the Bartoli indole synthesis allowed access to substituted indole cores that could then be subjected to a similar sequence of transformations as outlined in Scheme 6 or similar. Glyoxylation with oxalyl chloride and subsequent condensation with a secondary amine gives rise to glyoxamide intermediates such as 149. Reduction of such glyoxamides proved viable in accessing benzylprotected indolols that upon hydrogenolysis fashioned the desired compounds of general formula (I) (exemplified by P-85). One skilled in the art will recognise that utilising alternate secondary amines would allow access to differentially alkylated compounds of general formula (I) disclosed herein.

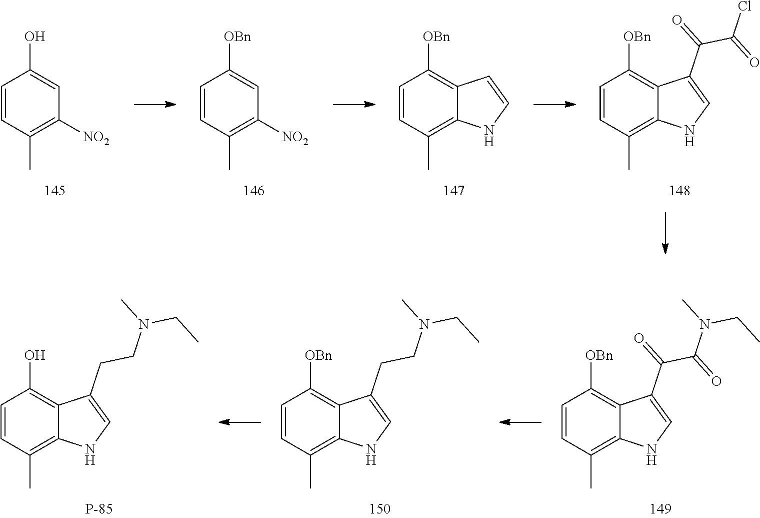

### Example 57: 3-(2-(ethyl(methyl)amino)ethyl)-7-methyl-1H-indol-4-ol (P-85)

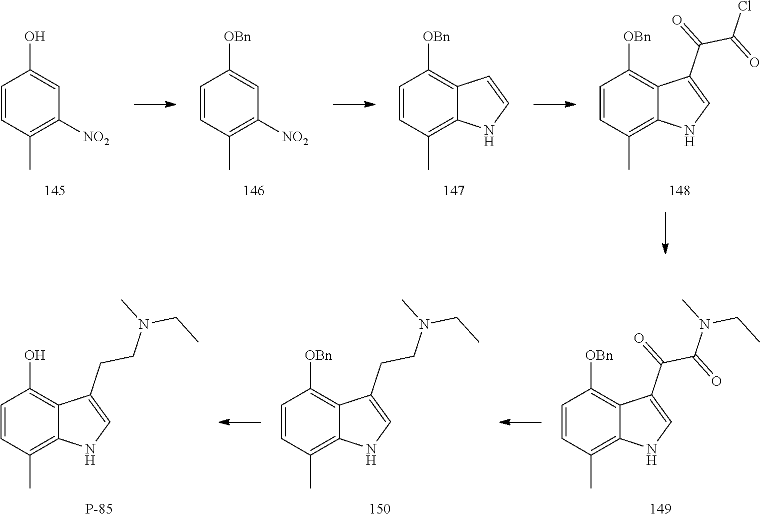

### Step 1: 4-(benzyloxy)-1-methyl-2-nitrobenzene (146)

To a stirred solution of 4-methyl-3-nitrophenol (10.0 g, 65.3 mmol) in DMF (300 mL) was added K₂CO₃ (22.6 g, 163 mmol) followed by benzyl bromide (8.53 mL, 71.8 mmol). The resulting reaction mixture was heated at 150° C. for 16 h. The reaction mixture was cooled to ambient temperature, diluted with H₂O (1.5 L), and extracted with EtOAc (250 mL×3). The organic layer was dried over Na₂SO₄, filtered, and the filtrate concentrated under reduced pressure. The residue was purified by column chromatography (SiO₂, hexane: EtOAc, v/v, 19:1 to 9:1) to afford the desired 4-(benzyloxy)-1-methyl-2-nitrobenzene (9.0 g, 57%) as a yellow solid. ¹H NMR (400 MHz, CDCl₃): δ 7.58-7.57 (d, J=2.4 Hz, 1H) 7.43-7.32 (m, 5H), 7.22 (d, J=8.8 Hz, 1H), 7.11 (dd, J=8.8, 2.4 Hz, 1H), 5.07 (s, 2H), 2.50 (s, 3H).

### Step 2: 4-(benzyloxy)-7-methyl-1H-indole (147)

To a stirred solution of 4-(benzyloxy)-1-methyl-2-nitrobenzene (9.0 g, 37.0 mmol) in anhydrous THF (370 mL) at −40° C. under an argon atmosphere was added dropwise vinyl magnesium bromide (1 M in THF, 222 mL, 220 mmol) and the reaction mixture was stirred for 4 h at −40° C. and then was allowed to warm to 0° C. The ice-cold reaction was quenched with saturated aqueous NH₄Cl solution (50 mL), diluted with H₂O (100 mL) and extracted with EtOAc (3×100 mL). The combined organic layers were washed subsequently with H₂O (100 mL), brine (150 mL) before being dried over Na₂SO₄, filtered, and the filtrate concentrated under reduced pressure. The residue was purified by column chromatography (SiO₂, hexane: EtOAc, v/v, 19:1 to 17:3) to afford 4-(benzyloxy)-7-methyl-1H-indole (2.3 g, 26%) as a grey solid. ¹H NMR (400 MHz, CDCl₃): δ 8.08 (br s, 1H), 7.50 (d, J=7.2 Hz, 2H), 7.39 (t, J=7.2 Hz, 2H), 7.34-7.28 (m, 1H), 7.16-7.12 (m, 1H), 6.87 (d, J=8.0 Hz, 1H), 6.74-6.72 (m, 1H), 6.50 (d, J=8.0 Hz, 1H), 5.21 (s, 2H), 2.43 (s, 3H).

### Step 3: 2-(4-(benzyloxy)-7-methyl-1H-indol-3-yl)-N-ethyl-N-methyl-2-oxoacetamide (149)

To an ice-cold solution of 4-(benzyloxy)-7-methyl-1H-indole (400 mg, 1.69 mmol) in anhydrous THF (5 mL) was added oxalyl chloride (0.43 mL, 5.04 mmol) at 0° C. The reaction mixture was then stirred at ambient temperature for 2 h. The reaction was cooled again and N-methylethanamine was added dropwise until the pH of the reaction solution was basic, at which point the reaction was stirred for 30 min before being quenched with H₂O (25 mL) and extracted with EtOAc (2×50 mL). The combined organic layers were washed with brine (20 mL), dried over anhydrous Na₂SO₄, filtered, and the filtrate concentrated under reduced pressure. The residue was purified by column chromatography (SiO₂, hexane: EtOAc, 6:4 to 5:5, v/v) to afford 2-(4-(benzyloxy)-7-methyl-1H-indol-3-yl)-N-ethyl-N-methyl-2-oxoacetamide (300 mg) as a brown solid which was used in the subsequent step without further purification. LCMS (ESI+): m/z 373 [M+Na]⁺.

### Step 4: 2-(4-(benzyloxy)-7-methyl-1H-indol-3-yl)-N-ethyl-N-methylethan-1-amine (150)

To an ice-cold solution of 2-(4-(benzyloxy)-7-methyl-1H-indol-3-yl)-N-ethyl-N-methyl-2-oxoacetamide (300 mg) in anhydrous THF (20 mL) was added 1 M LiAlH₄ in THF (3.42 mL, 3.42 mmol). The reaction mixture was stirred at 0° C. for 30 min, and then at reflux for 16 h. The reaction mixture was cooled to 0° C., quenched with saturated aq. Na₂SO₄ solution (3 mL), diluted with CH₂Cl₂ (25 mL), and finally silica gel (100 mg) was added. The resulting suspension was stirred for 30 min, then filtered through a celite pad and the filter cake was washed with MeOH (20 mL). The combined filtrate was concentrated under reduced pressure to afford 2-(4-(benzyloxy)-7-methyl-1H-indol-3-yl)-N-ethyl-N-methylethan-1-amine (150 mg) as a brown solid which was used in the subsequent step without further purification. LCMS (ESI+): m/z 323 [M+H]⁺.

### Step 5: 3-(2-(ethyl(methyl)amino)ethyl)-7-methyl-1H-indol-4-ol (P-85)

A stirred solution of 2-(4-(benzyloxy)-7-methyl-1H-indol-3-yl)-N-ethyl-N-methylethan-1-amine (150 mg) in degassed MeOH (15 mL) and formic acid (0.3 mL) was treated with Pd/C (120 mg, 10% w/w) at ambient temperature. The reaction mixture was stirred under H₂ gas at ambient pressure and temperature for 16 h. The reaction mixture was filtered through a celite pad and washed with MeOH (20 mL). The combined filtrate was concentrated under reduced pressure and the resultant residue was purified by reverse phase column chromatography (C18, 0.01% v/v HCO₂H in MeCN), followed by lyophilization, to afford 3-(2-(ethyl(methyl)amino)ethyl)-7-methyl-1H-indol-4-ol (16 mg, 4% over 3 steps) as an off-white solid. ¹H NMR (400 MHZ, DMSO-d₆): δ 10.50 (br s, 1H), 6.92 (s, 1H), 6.57 (d, J=7.2 Hz, 1H), 6.16 (d, J=7.2 Hz, 1H), 2.86 (t, J=6.8 Hz, 2H), 2.59 (t, J=6.8 Hz, 2H), 2.41 (q, J=7.2 Hz, 2H), 2.22 (s, 3H), 2.18 (s, 3H), 0.96 (t, J=7.2 Hz, 3H). LCMS (ESI+): m/z 233.2 [M+H]⁺. HPLC Purity (PDA): 95.0%

### Example 58: 7-methyl-3-(2-(methyl(propyl)amino)ethyl)-1H-indol-4-ol (P-86)

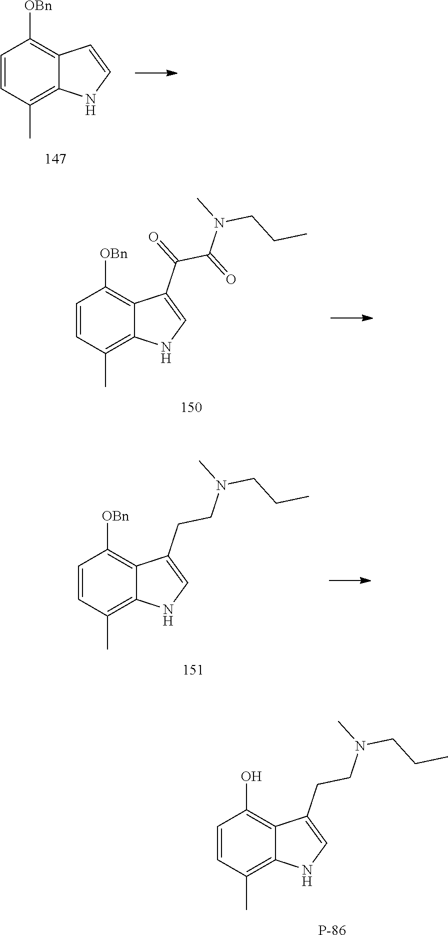

### Step 1: 2-(4-(benzyloxy)-7-methyl-1H-indol-3-yl)-N-methyl-2-oxo-N-propylacetamide (150)

To an ice-cold solution of 4-(benzyloxy)-7-methyl-1H-indole (400 mg, 1.69 mmol) in anhydrous THF (5 mL) was added oxalyl chloride (0.43 mL, 5.04 mmol) at 0° C. The reaction mixture was then stirred at ambient temperature for 2 h. The reaction was cooled again and N-methylpropylamine was added dropwise until the pH of the reaction solution was basic, at which point the reaction was stirred for 30 min before being quenched with H₂O (25 mL) and extracted with EtOAc (2×50 mL). The combined organic layers were washed with brine (20 mL), dried over anhydrous Na₂SO₄, filtered, and the filtrate concentrated under reduced pressure. The residue was purified by column chromatography (SiO₂, hexane: EtOAc, 6:4 to 5:5, v/v) to afford 2-(4-(benzyloxy)-7-methyl-1H-indol-3-yl)-N-methyl-2-oxo-N-propylacetamide (620 mg) as a brown solid which was used in the subsequent step without further purification. LCMS (ESI+): m/z 365.2 [M+H]⁺.

### Step 2: N-(2-(4-(benzyloxy)-7-methyl-1H-indol-3-yl)ethyl)-N-methylpropan-1-amine (151)

To an ice-cold solution of 2-(4-(benzyloxy)-7-methyl-1H-indol-3-yl)-N-methyl-2-oxo-N-propylacetamide (600 mg) in anhydrous THF (20 mL) was added 1 M LiAlH₄ in THF (6.59 mL, 6.59 mmol). The reaction mixture was stirred at 0° C. for 30 min, and then at reflux for 16 h. The reaction mixture was cooled to 0° C., quenched with saturated aq. Na₂SO₄ solution (3 mL), diluted with CH₂Cl₂ (25 mL), and finally silica gel (100 mg) was added. The resulting suspension was stirred for 30 min, then filtered through a celite pad and the filter cake was washed with MeOH (20 mL). The combined filtrate was concentrated under reduced pressure to afford N-(2-(4-(benzyloxy)-7-methyl-1H-indol-3-yl)ethyl)-N-methylpropan-1-amine (400 mg) as a brown oil which was used in the subsequent step without further purification. LCMS (ESI+): m/z 337.7 [M+H]⁺.

### Step 3: 3-(2-(methyl(propyl)amino)ethyl)-7-methyl-1H-indol-4-ol (P-86)

A stirred solution of N-(2-(4-(benzyloxy)-7-methyl-1H-indol-3-yl)ethyl)-N-methylpropan-1-amine (400 mg) in MeOH (15 mL) and formic acid (0.3 mL) was purged with N₂ gas for 15 min, before Pd/C (120 mg) was added at ambient temperature. The reaction mixture was stirred under H₂ gas at ambient pressure and temperature for 16 h. The reaction mixture was filtered through a celite pad and washed with MeOH (20 mL). The combined filtrate was concentrated under reduced pressure in a bath temperature not exceeding 35° C. The residue was purified by reverse phase column chromatography (C18, 0.01% v/v HCO₂H in MeCN), followed by lyophilization, to afford 3-(2-(methyl(propyl)amino)ethyl)-7-methyl-1H-indol-4-ol (50 mg, 12% over 3 steps) as a brown solid. ¹H NMR (400 MHZ, DMSO-d₆): δ 10.54 (br, 1H), 8.21 (br, 1H), 6.94 (s, 1H), 6.58 (d, J=7.5 Hz, 1H), 6.18 (d, J=7.5 Hz, 1H), 2.92 (t, J=6.8 Hz, 3H), 2.72 (t, J=6.8 Hz, 2H), 2.43-2.41 (m, 2H), 2.33 (s, 3H), 2.28 (s, 3H), 1.45 (q, J=7.4 Hz, 2H), 0.83 (t, J=7.3 Hz, 3H). LCMS: m/z: 247.3 [M+H]⁺. HPLC Purity (PDA): 98.9%.

### Example 59: 3-(2-(isopropyl(methyl)amino)ethyl)-7-methyl-1H-indol-4-ol (P-87)

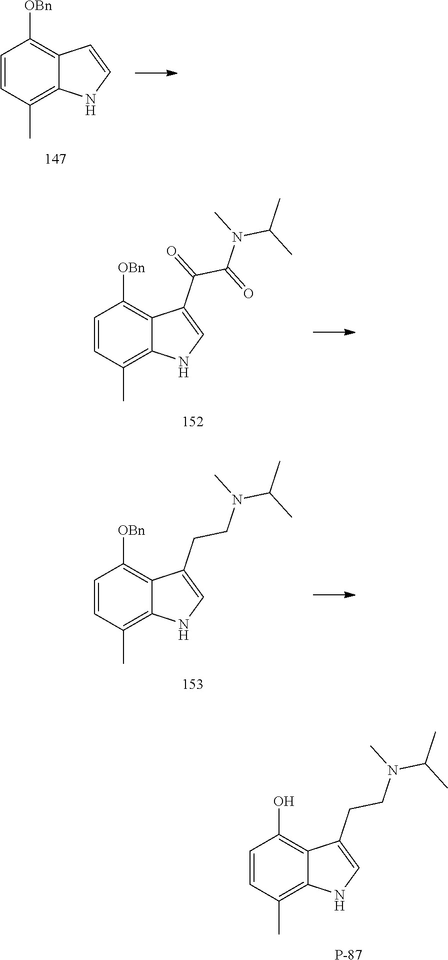

### Step 1: 2-(4-(benzyloxy)-7-methyl-1H-indol-3-yl)-N-isopropyl-N-methyl-2-oxoacetamide (152)

To an ice-cold solution of 4-(benzyloxy)-7-methyl-1H-indole (400 mg, 1.69 mmol) in anhydrous THF (5 mL) was added oxalyl chloride (0.43 mL, 5.04 mmol) at 0° C. The reaction mixture was then stirred at ambient temperature for 2 h. The reaction was cooled again, and N-methylpropan-2-amine was added dropwise until the pH of the reaction solution was basic, at which point the reaction was stirred for 30 min before being quenched with H₂O (25 mL) and extracted with EtOAc (2×50 mL). The combined organic layers were washed with brine (20 mL), dried over anhydrous Na₂SO₄, filtered, and the filtrate concentrated under reduced pressure. The residue was purified by column chromatography (SiO₂, hexane: EtOAc, 6:4 to 5:5, v/v) to afford 2-(4-(benzyloxy)-7-methyl-1H-indol-3-yl)-N-isopropyl-N-methyl-2-oxoacetamide (600 mg) as a brown solid which was used in the subsequent step without further purification. LCMS (ESI+): m/z 365 [M+H]⁺.

### Step 2: N-(2-(4-(benzyloxy)-7-methyl-1H-indol-3-yl)ethyl)-N-methylpropan-2-amine (153)

To an ice-cold solution of 2-(4-(benzyloxy)-7-methyl-1H-indol-3-yl)-N-isopropyl-N-methyl-2-oxoacetamide (600 mg) in anhydrous THF (20 mL) was added 1 M LiAlH₄ in

THF (6.59 mL, 6.59 mmol). The reaction mixture was stirred at 0° C. for 30 min, and then at reflux for 16 h. The reaction mixture was cooled to 0° C., quenched with saturated aq. Na₂SO₄ solution (3 mL), diluted with CH₂Cl₂ (25 mL), and finally silica gel (100 mg) was added. The resulting suspension was stirred for 30 min, then filtered through a celite pad and the filter cake was washed with MeOH (20 mL). The combined filtrate was concentrated under reduced pressure to afford N-(2-(4-(benzyloxy)-7-methyl-1H-indol-3-yl)ethyl)-N-methylpropan-2-amine (410 mg) as a brown oil which was used in the subsequent step without further purification. LCMS (ESI+): m/z 337 [M+H]⁺.

### Step 3: 3-(2-(isopropyl(methyl)amino)ethyl)-7-methyl-1H-indol-4-ol (P-87)

A stirred solution of N-(2-(4-(benzyloxy)-7-methyl-1H-indol-3-yl)ethyl)-N-methylpropan-2-amine (400 mg) in MeOH (15 mL) and formic acid (0.3 mL) was purged with N₂ gas for 15 min, before Pd/C (120 mg, 10% w/w) was added at ambient temperature. The reaction mixture was stirred under H₂ gas at ambient pressure and temperature for 16 h. The reaction mixture was filtered through a celite pad and washed with MeOH (20 mL). The combined filtrate was concentrated under reduced pressure and the residue was purified by reverse phase column chromatography (C18, 0.01% v/v HCO₂H in MeCN), followed by lyophilization, to afford 3-(2-(isopropyl(methyl)amino)ethyl)-7-methyl-1H-indol-4-ol (50 mg, 12% over 3 steps) as a brown solid. ¹H NMR (400 MHZ, DMSO-d₆): δ 10.58 (br s, 1H), 8.28 (s, 1H), 6.96 (s, 1H), 6.58 (d, J=7.2 Hz, 1H), 6.19 (d, J=7.6 Hz, 1H), 3.05 (sept, J=6.4 Hz, 1H), 2.95 (t, J=6.4 Hz, 2H), 2.82 (t, J=6.4 Hz, 2H), 2.39 (s, 3H), 2.28 (s, 3H), 1.01 (d, J=6.4 Hz, 6H). LCMS: m/z: 247.2 [M+H]⁺. HPLC Purity (PDA): 95.0%

### Example 60: 3-(2-(diethylamino)ethyl)-7-methyl-1H-indol-4-ol (P-88)

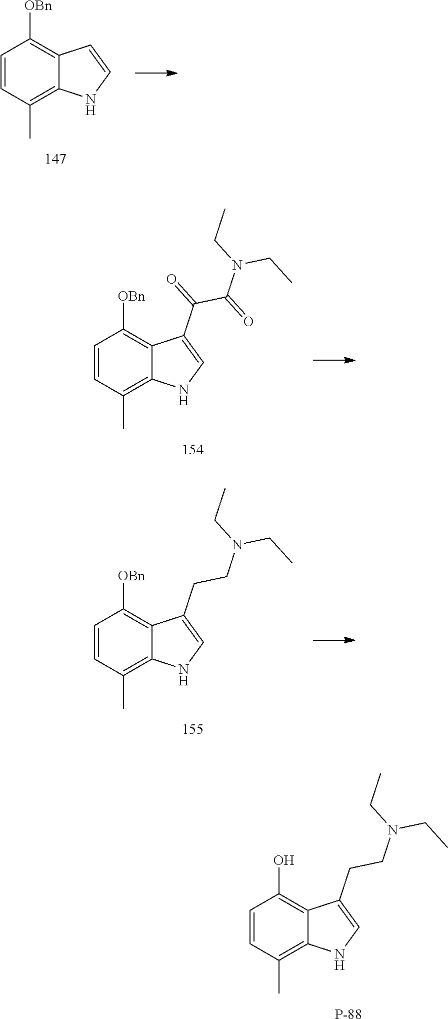

### Step 1: 2-(4-(benzyloxy)-7-methyl-1H-indol-3-yl)-N,N-diethyl-2-oxoacetamide (154)

To an ice-cold solution of 4-(benzyloxy)-7-methyl-1H-indole (400 mg, 1.69 mmol) in anhydrous THF (5 mL) was added oxalyl chloride (0.43 mL, 5.04 mmol) at 0° C. The reaction mixture was then stirred at ambient temperature for 2 h. The reaction was cooled again and diethylamine was added dropwise until the pH of the reaction solution was basic, at which point the reaction was stirred for 30 min before being quenched with H₂O (25 mL) and extracted with EtOAc (2×50 mL). The combined organic layers were washed with brine (20 mL), dried over anhydrous Na₂SO₄, filtered, and the filtrate concentrated under reduced pressure. The residue was purified by column chromatography (SiO₂, hexane: EtOAc, 6:4 to 5:5, v/v) to afford 2-(4-(benzyloxy)-7-methyl-1H-indol-3-yl)-N,N-diethyl-2-oxoacetamide (560 mg) as a brown solid which was used in the subsequent step without further purification. LCMS (ESI+): m/z 365.3 [M+H]⁺.

### Step 2: 2-(4-(benzyloxy)-7-methyl-1H-indol-3-yl)-N,N-diethylethan-1-amine (155)

To an ice-cold solution of 2-(4-(benzyloxy)-7-methyl-1H-indol-3-yl)-N,N-diethyl-2-oxoacetamide (560 mg) in anhydrous THF (20 mL) was added 1 M LiAlH₄ in THF (6.15 mL, 6.15 mmol). The reaction mixture was stirred at 0° C. for 30 min, and then at reflux for 16 h. The reaction mixture was cooled to 0° C., quenched with saturated aq. Na₂SO₄ solution (3 mL), diluted with CH₂Cl₂ (25 mL), and finally silica gel (100 mg) was added. The resulting suspension was stirred for 30 min, then filtered through a celite pad and the filter cake was washed with MeOH (20 mL). The combined filtrate was concentrated under reduced pressure to afford 2-(4-(benzyloxy)-7-methyl-1H-indol-3-yl)-N,N-diethylethan-1-amine (400 mg) as a brown oil which was used in the subsequent step without further purification. LCMS (ESI+): m/z 337.3 [M+H]⁺.

### Step 3: 3-(2-(diethylamino)ethyl)-7-methyl-1H-indol-4-ol (P-88)

A stirred solution of 2-(4-(benzyloxy)-7-methyl-1H-indol-3-yl)-N,N-diethylethan-1-amine (400 mg) in MeOH (15 mL) and formic acid (0.3 mL) was purged with N₂ gas for 15 min, before Pd/C (120 mg) was added at ambient temperature. The reaction mixture was stirred under H₂ gas at ambient pressure and temperature for 16 h. The reaction mixture was filtered through a celite pad and washed with MeOH (20 mL). The combined filtrate was concentrated under reduced pressure and the residue was purified by reverse phase column chromatography (C18, 0.01% v/v HCO₂H in MeCN), followed by lyophilization, to afford 3-(2-(diethylamino)ethyl)-7-methyl-1H-indol-4-ol (55 mg, 13% over 3 steps) as a brown solid. ¹H NMR (400 MHZ, DMSO-d₆): δ 10.54 (br s, 1H), 8.22 (br s, 1H), 6.94 (s, 1H), 6.58 (d, J=7.6 Hz, 1H), 6.18 (d, J=7.6 Hz, 1H), 2.89 (t, J=6.4 Hz, 2H), 2.77 (t, J=6.4 Hz, 2H), 2.65 (q, J=7.2 Hz, 4H), 2.28 (s, 3H), 1.00 (t, J=7.2 Hz, 6H). LCMS: m/z: 247.2 [M+H]⁺. HPLC Purity (PDA): 95.0%

### Example 61: 3-(2-(dipropylamino)ethyl)-7-methyl-1H-indol-4-ol (P-89)

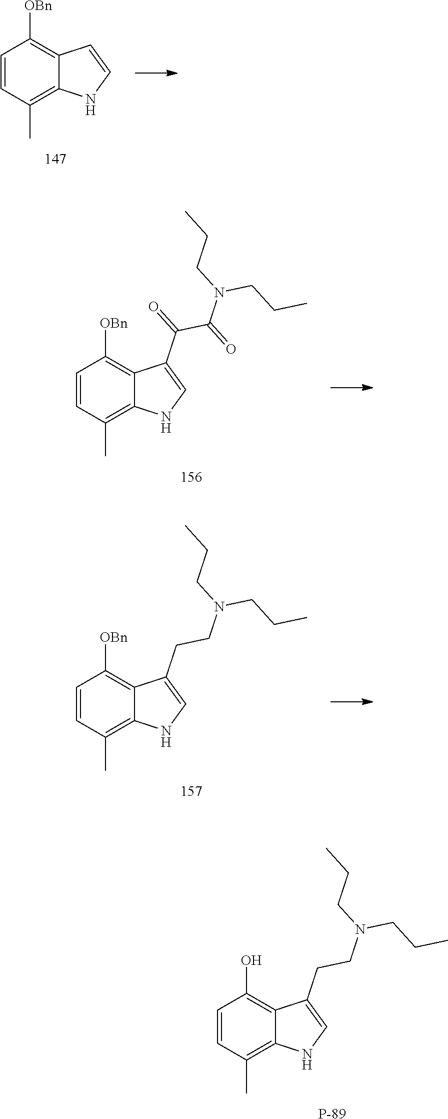

### Step 1: 2-(4-(benzyloxy)-7-methyl-1H-indol-3-yl)-N,N-dipropyl-2-oxoacetamide (156)

To an ice-cold solution of 4-(benzyloxy)-7-methyl-1H-indole (400 mg, 1.69 mmol) in anhydrous THF (5 mL) was added oxalyl chloride (0.43 mL, 5.04 mmol) at 0° C. The reaction mixture was then stirred at ambient temperature for 2 h. The reaction was cooled again and dipropylamine was added dropwise until the pH of the reaction solution was basic, at which point the reaction was stirred for 30 min before being quenched with H₂O (25 mL) and extracted with EtOAc (2×50 mL). The combined organic layers were washed with brine (20 mL), dried over anhydrous Na₂SO₄, filtered, and the filtrate concentrated under reduced pressure. The residue was purified by column chromatography (SiO₂, hexane: EtOAc, 6:4 to 5:5, v/v) to afford 2-(4-(benzyloxy)-7-methyl-1H-indol-3-yl)-N,N-dipropyl-2-oxoacetamide (610 mg) as a brown solid which was used in the subsequent step without further purification. LCMS (ESI+): m/z 415.3 [M+Na]⁺.

### Step 2: 2-(4-(benzyloxy)-7-methyl-1H-indol-3-yl)-N,N-dipropylethan-1-amine (157)

To an ice-cold solution of 2-(4-(benzyloxy)-7-methyl-1H-indol-3-yl)-N,N-dipropyl-2-oxoacetamide (600 mg) in anhydrous THF (20 mL) was added 1 M LiAlH₄ in THF (6.11 mL, 6.11 mmol). The reaction mixture was stirred at 0° C. for 30 min, and then at reflux for 16 h. The reaction mixture was cooled to 0° C., quenched with saturated aq. Na₂SO₄ solution (3 mL), diluted with CH₂Cl₂ (25 mL), and finally silica gel (100 mg) was added. The resulting suspension was stirred for 30 min, then filtered through a celite pad and the filter cake was washed with MeOH (20 mL). The combined filtrate was concentrated under reduced pressure to afford 2-(4-(benzyloxy)-7-methyl-1H-indol-3-yl)-N,N-dipropylethan-1-amine (410 mg) as a brown solid which was used in the subsequent step without further purification. LCMS (ESI+): m/z 365 [M+H]⁺.

### Step 3: 3-(2-(dipropylamino)ethyl)-7-methyl-1H-indol-4-ol (P-89)

A stirred solution of 2-(4-(benzyloxy)-7-methyl-1H-indol-3-yl)-N,N-dipropylethan-1-amine (400 mg) in MeOH (15 mL) and formic acid (0.3 mL) was purged with N₂ gas for 15 min, before Pd/C (120 mg, 10% w/w) was added at ambient temperature. The reaction mixture was stirred under H₂ gas at ambient pressure and temperature for 16 h. The reaction mixture was filtered through a celite pad and washed with MeOH (20 mL). The combined filtrate was concentrated under reduced pressure and the residue was purified by reverse phase column chromatography (C18, 0.01% v/v HCO₂H in MeCN), followed by lyophilization, to afford 3-(2-(dipropylamino)ethyl)-7-methyl-1H-indol-4-ol (55 mg, 12% over 3 steps) as an off-white solid. ¹H NMR (400 MHZ, DMSO-d₆): δ 10.52 (s, 1H), 8.22 (s, 1H), 6.93 (s, 1H), 6.57 (d, J=7.6 Hz, 1H), 6.18 (d, J=8.0 Hz, 1H), 2.90 (t, J=6.8 Hz, 2H), 2.77 (t, J=6.8 Hz, 2H), 2.55-2.50 (m, 4H), 2.28 (s, 3H), 1.45 (q, J=7.2 Hz, 4H), 0.83 (t, J=7.2 Hz, 6H). LCMS (ESI+): m/z: 275.1 [M+H]⁺. HPLC Purity (PDA): 95.0%

### Example 62: 3-(2-(diisopropylamino)ethyl)-7-methyl-1H-indol-4-ol (P-90)

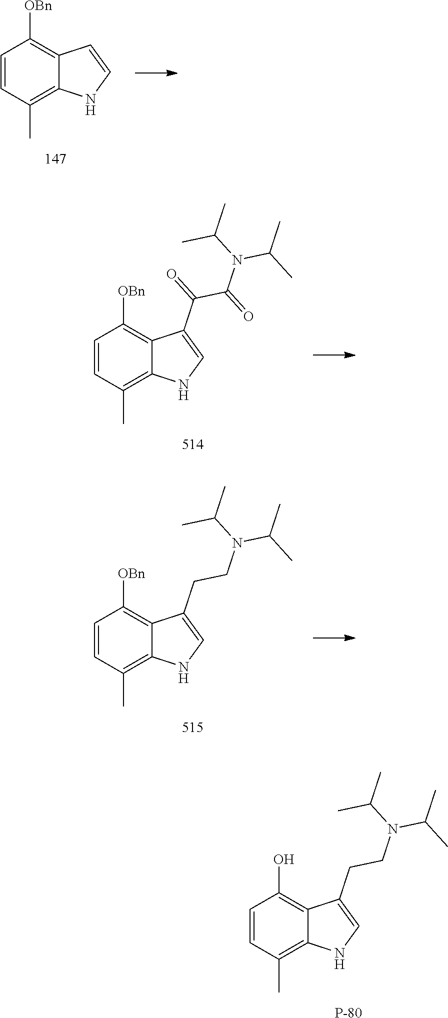

### Step 1: 2-(4-(benzyloxy)-7-methyl-1H-indol-3-yl)-N,N-diisopropyl-2-oxoacetamide (514)

To an ice-cold solution of 4-(benzyloxy)-7-methyl-1H-indole (400 mg, 1.69 mmol) in anhydrous THF (5 mL) was added oxalyl chloride (0.43 mL, 5.04 mmol) at 0° C. The reaction mixture was then stirred at ambient temperature for 2 h. The reaction was cooled again and dipropylamine was added dropwise until the pH of the reaction solution was basic, at which point the reaction was stirred for 30 min before being quenched with H₂O (25 mL) and extracted with EtOAc (2×50 mL). The combined organic layers were washed with brine (20 mL), dried over anhydrous Na₂SO₄, filtered, and the filtrate concentrated under reduced pressure. The residue was purified by column chromatography (SiO₂, hexane: EtOAc, 6:4 to 5:5, v/v) to afford 2-(4-(benzyloxy)-7-methyl-1H-indol-3-yl)-N,N-diisopropyl-2-oxoacetamide (600 mg) as a white solid which was used in the subsequent step without further purification. LCMS (ESI+): m/z 393.3 [M+H]⁺.

### Step 2: N-(2-(4-(benzyloxy)-7-methyl-1H-indol-3-yl)ethyl)-N-isopropylpropan-2-amine (515)

To an ice-cold solution of 2-(4-(benzyloxy)-7-methyl-1H-indol-3-yl)-N,N-diisopropyl-2-oxoacetamide (600 mg) in anhydrous THF (20 mL) was added 1 M LiAlH₄ in THF (6.11 mL, 6.11 mmol). The reaction mixture was stirred at 0° C. for 30 min, and then at reflux for 16 h. The reaction mixture was cooled to 0° C., quenched with saturated aq. Na₂SO₄ solution (3 mL), diluted with CH₂Cl₂ (25 mL), and finally silica gel (100 mg) was added. The resulting suspension was stirred for 30 min, then filtered through a celite pad and the filter cake was washed with MeOH (20 mL). The combined filtrate was concentrated under reduced pressure to afford N-(2-(4-(benzyloxy)-7-methyl-1H-indol-3-yl)ethyl)-N-isopropylpropan-2-amine (390 mg) as a white solid which was used in the subsequent step without further purification. LCMS (ESI+): m/z 365.8 [M+H]⁺.

### Step 3: 3-(2-(diisopropylamino)ethyl)-7-methyl-1H-indol-4-ol (P-90)

A stirred solution of N-(2-(4-(benzyloxy)-7-methyl-1H-indol-3-yl)ethyl)-N-isopropylpropan-2-amine (390 mg) in MeOH (15 mL) and formic acid (0.3 mL) was purged with N₂ gas for 15 min, before Pd/C (120 mg) was added at ambient temperature. The reaction mixture was stirred under H₂ gas at ambient pressure and temperature for 16 h. The reaction mixture was filtered through a celite pad and washed with MeOH (20 mL). The combined filtrate was concentrated under reduced pressure in a bath temperature not exceeding 35° C. The residue was purified by reverse phase column chromatography (C18, 0.01% v/v HCO₂H in MeCN), followed by lyophilization, to afford 3-(2-(diisopropylamino)ethyl)-7-methyl-1H-indol-4-ol (45 mg, 10% over 3 steps) as an off-white solid. ¹H NMR (400 MHZ, DMSO-d₆): δ 10.53 (br s, 1H), 8.25 (s, 1H), 6.93 (s, 1H), 6.57 (d, J=7.2 Hz, 1H), 6.18 (d, J=7.6 Hz, 1H), 3.17-3.10 (m, 2H), 2.87 (t, J=6.8 Hz, 2H), 2.77 (t, J=6.4 Hz, 2H), 2.28 (s, 3H), 1.0 (d, J=6.4 Hz, 12H). LCMS: m/z: 275.3 [M+H]⁺. HPLC Purity (PDA): 95.0%

Scheme 14: Compounds of general formula (I) can be synthesised from the appropriately substituted benzaldehyde following a sequence of synthetic steps outlined in Scheme 14 or similar as one skilled in the art may consider. Initial Knovenagel condensation of substituted benzaldehydes such as 158 with ethyl azidoacetate gives rise to vinyl azide 159 which can undergo a thermally induced intramolecular cyclisation to furnish indole 160. Standard ester hydrolysis and subsequent thermal decarboxylation gave indole 162. Such indole cores could then be condensed with dimethylamino-2-nitroethylene to give nitrovinyl indoles. Reduction of such indoles proved viable in accessing unsubstituted ethyl amine analogues that could subsequently undergo reductive alkylation to access compounds of general formula (I) (exemplified by P-91). One skilled in the art will recognise that protecting the amine with a suitable protecting group such as benzyl, followed by alkylation, subsequent deprotection, and a second alkylation would allow access to differentially alkylated compounds of general formula (I) disclosed herein.

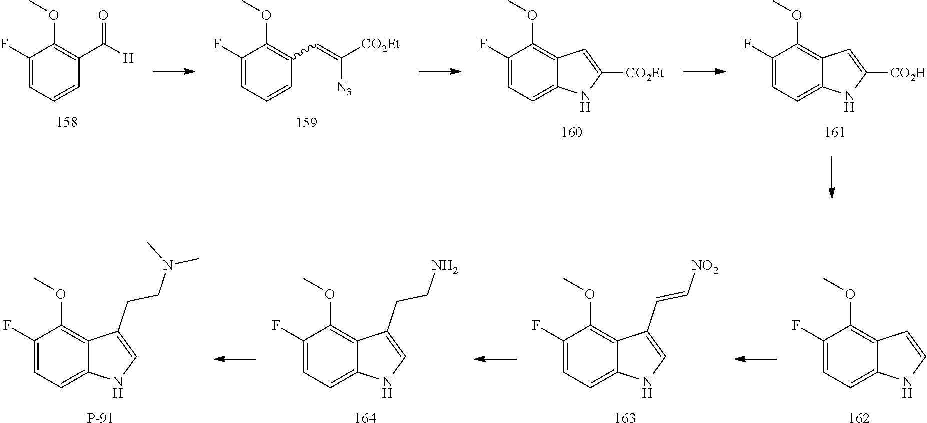

### Example 63: 2-(5-fluoro-4-methoxy-1H-indol-3-yl)-N,N-dimethylethan-1-amine (P-91)

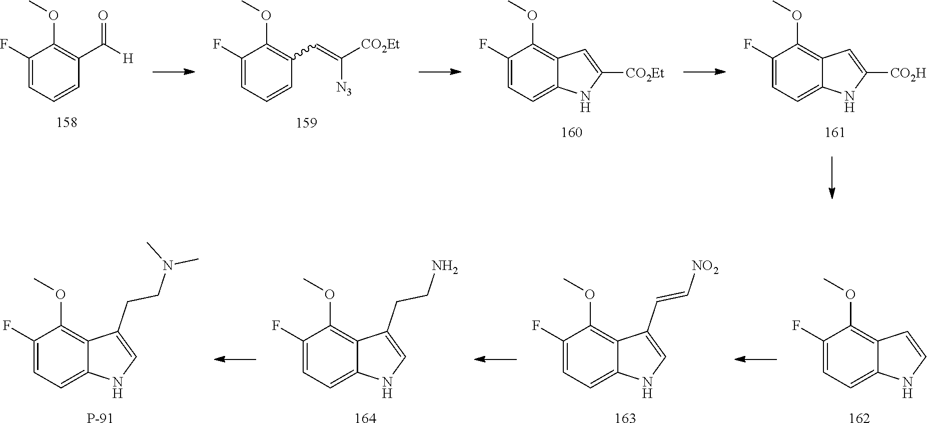

### Step 1: ethyl 2-azido-3-(3-fluoro-2-methoxyphenyl)acrylate (159)

A solution of 3-fluoro-2-methoxybenzaldehyde (3.0 g, 19.5 mmol) and ethyl 2-azidoacetate (7.54 g, 3 eq., 58.4 mmol) in EtOH (15 mL) was treated with 22 wt. % NaOEt (18.1 g, 3 eq., 58.4 mmol) at −20° C. and then stirred at 0° C. for 2 h. Upon completion, the reaction mixture was poured into saturated aq. NH₄Cl solution (100 mL) and then extracted with EtOAc (50 mL×2). The combined organic layer was washed with brine (30 mL), dried over anhydrous Na₂SO₄, filtered, and the filtrate concentrated in vacuo. The residue was purified by column chromatography (SiO₂, petroleum ether/EtOAc, v/v, 50/1 to 20/1) to afford a crude ethyl 2-azido-3-(3-fluoro-2-methoxyphenyl) prop-2-enoate (5.0 g) as a light yellow solid.

### Step 2: ethyl 5-fluoro-4-methoxy-1H-indole-2-carboxylate (160)

A solution of crude ethyl 2-azido-3-(3-fluoro-2-methoxyphenyl) prop-2-enoate (5.0 g) in xylene (30 mL) was stirred at 130° C. for 1 h. The reaction mixture was concentrated in vacuo and the residue was purified by column chromatography (SiO₂, petroleum ether/EtOAc, v/v, 50/1 to 20/1) to afford ethyl 5-fluoro-4-methoxy-1H-indole-2-carboxylate (1.5 g, 32% over 2 steps) as a white solid. ¹H NMR (400 MHZ, CDCl₃): δ 8.86 (br. s, 1H), 7.35 (d, J=1.6 Hz, 1H), 7.11 (dd, J=11.6, 8.8 Hz, 1H), 7.02 (dd, J=8.8, 3.6 Hz, 1H), 4.41 (q, J=7.2 Hz, 2H), 4.15 (d, J=2.0 Hz, 3H), 1.43 (t, J=7.2 Hz, 3H).

### Step 3: 5-fluoro-4-methoxy-1H-indole-2-carboxylic acid (161)

A solution of ethyl 5-fluoro-4-methoxy-1H-indole-2-carboxylate (1.5 g, 6.32 mmol) in EtOH (6 mL) and H₂O (15 mL) was treated with KOH (887 mg, 15.8 mmol) and the mixture was then stirred at 60° C. for 4 h. The volatile components were removed in vacuo and the residue was diluted with H₂O (20 mL). The pH was adjusted to 3 using 1 M aq. HCl solution, and then extracted with EtOAc (10 mL×2). The combined organic layers were washed with brine (10 mL), dried over anhydrous Na₂SO₄, filtered, and the filtrate concentrated in vacuo to afford 5-fluoro-4-methoxy-1H-indole-2-carboxylic acid (1.0 g, 76%) as white solid. ¹H NMR (400 MHZ, MeOD-d₄): δ 7.23 (s, 1H), 7.09-7.02 (m, 2H), 4.09 (d, J=1.6 Hz, 3H).

### Step 4: 5-fluoro-4-methoxy-1H-indole (162)

A solution of 5-fluoro-4-methoxy-1H-indole-2-carboxylic acid (1.0 g, 4.78 mmol) in quinoline (10 mL) was treated with copper (304 mg, 4.78 mmol) and the resulting mixture was stirred at 230° C. for 2 h under N₂. The cooled reaction mixture was diluted with EtOAc (60 mL) and washed sequentially with 1 M aq. HCl solution (30 mL×3), saturated aq. NaHCO₃ solution (30 mL×2), brine (20 mL), and then dried over anhydrous Na₂SO₄, filtered, and the filtrate concentrated in vacuo. The residue was purified by column chromatography (SiO₂, petroleum ether/EtOAc, v/v, 100/1 to 50/1) to afford 5-fluoro-4-methoxy-1H-indole (440 mg, 56%) as a yellow oil. ¹H NMR (400 MHz, CDCl₃): δ 8.14 (br. s, 1H), 7.19 (m, 1H), 7.03-6.94 (m, 2H), 6.68 (m, 1H), 4.13 (d, J=1.6 Hz, 3H).

### Step 5: (E)-5-fluoro-4-methoxy-3-(2-nitrovinyl)-1H-indole (163)

A solution of 5-fluoro-4-methoxy-1H-indole (430 mg, 2.60 mmol) in TFA (2.6 mL) was treated with (E)-N,N-dimethyl-2-nitroethen-1-amine (302 mg, 2.6 mmol) and the mixture was stirred at 20° C. for 1 h. The mixture was adjusted to pH 8 with saturated aqueous NaHCO₃ solution and then extracted with EtOAc (30 mL×2). The combined organic layers were washed with brine (10 mL), dried over anhydrous Na₂SO₄, filtered, and the filtrate concentrated in vacuo. The residue was triturated with petroleum ether/EtOAc (v/v, 10/1, 10 mL) at 20° C. for 30 min to give (E)-5-fluoro-4-methoxy-3-(2-nitrovinyl)-1H-indole (550 mg, 89%) as red solid. ¹H NMR (400 MHZ, DMSO-d₆): δ 12.40-12.30 (br s, 1H), 8.49 (d, J=13.2 Hz, 1H), 8.35 (s, 1H), 8.05 (d, J=13.2 Hz, 1H), 7.26-7.12 (m, 2H), 3.98 (s, 3H).

### Step 6: 2-(5-fluoro-4-methoxy-1H-indol-3-yl)ethan-1-amine (164)

To an ice-cold solution of (E)-5-fluoro-4-methoxy-3-(2-nitrovinyl)-1H-indole (0.45 g, 1.91 mmol) in THF (4.5 mL) was added LiAlH₄ (795 mg, 20.9 mmol) in portions and then the mixture was stirred at 80° C. for 2 h. The mixture was cooled to 0° C., diluted with THF (20 mL), quenched with Na₂SO₄·10H₂O (3.00 g), stirred at 20° C. for 30 min, then filtered. The filtrate was concentrated in vacuo to give 2-(5-fluoro-4-methoxy-1H-indol-3-yl)ethan-1-amine (360 mg, 91%) as a brown oil. ¹H NMR (400 MHZ, MeOD-d₄): δ 7.03 (s, 1H), 6.98 (dd, J=8.8, 3.6 Hz, 1H), 6.90-6.85 (m, 1H), 4.00 (d, J=2.0 Hz, 3H), 3.04-2.94 (m, 4H). LCMS (ESI+): m/z 209.1 [M+H]⁺.

### Step 7: 2-(5-fluoro-4-methoxy-1H-indol-3-yl)-N,N-dimethylethan-1-amine (P-91)

A solution of 2-(5-fluoro-4-methoxy-1H-indol-3-yl)ethan-1-amine (180 mg, 0.86 mmol) in MeOH (3.6 mL) at 0° C. was treated with AcOH (208 mg, 3.46 mmol), NaBH₃CN (109 mg, 1.73 mmol), and 37% w/w aqueous formaldehyde (175 mg, 2.16 mmol) and then stirred at 20° C. for 3 h. The mixture was adjusted to pH 8 with saturated aqueous Na₂CO₃ and the organic solvent evaporated in vacuo. The residue was diluted with H₂O (20 mL) and extracted with EtOAc (100 mL×2). The combined organic layers were washed with brine (10 mL), dried over anhydrous Na₂SO₄, filtered, and the filtrate concentrated in vacuo. The residue was purified by preparative HPLC (column:

Phenomenex C18 80×40 mm×3 μm; mobile phase: [water (NH₄HCO₃)-ACN]; B: 5-35%, 8 min) to give 2-(5-fluoro-4-methoxy-1H-indol-3-yl)-N,N-dimethylethan-1-amine (75 mg, 37%) as an off-white solid. ¹H NMR (400 MHZ, MeOD-d₄): δ 7.02-6.94 (m, 2H), 6.86 (dd, J=12.0, 8.8 Hz, 1H), 4.01 (s, 3H), 3.05-2.98 (m, 2H), 2.75-2.67 (m, 2H), 2.39 (s, 6H). LCMS (ESI+): m/z 237.2 [M+H]⁺. HPLC Purity (220 nm): 100%.

### Example 64: N-ethyl-2-(5-fluoro-4-methoxy-1H-indol-3-yl)-N-methylethan-1-amine (P-92)

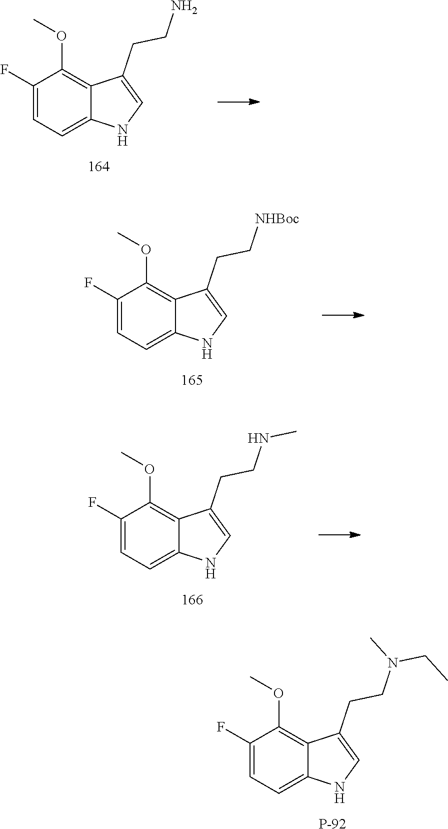

### Step 1: tert-butyl (2-(5-fluoro-4-methoxy-1H-indol-3-yl)ethyl)carbamate (165)

To a solution of 2-(5-fluoro-4-methoxy-1H-indol-3-yl)ethan-1-amine (1.00 g, 4.80 mmol) in THF (7 mL) was added Boc₂O (1.10 g, 5.04 mmol). The mixture was stirred at 25° C. for 2 h. The mixture was concentrated under reduced pressure and the residue was purified by preparative TLC (SiO₂, petroleum ether/EtOAc, v/v, 3/1) to give crude tert-butyl (2-(5-fluoro-4-methoxy-1H-indol-3-yl)ethyl)carbamate (800 mg) as a brown oil which was used in the subsequent step without further purification.

### Step 2: 2-(5-fluoro-4-methoxy-1H-indol-3-yl)-N-methylethan-1-amine (166)

To an ice-cold (0° C.) solution of crude tert-butyl (2-(5-fluoro-4-methoxy-1H-indol-3-yl)ethyl)carbamate (800 mg) in THF (5.60 mL) was added LiAlH₄ (2.5 M in THF, 5.19 mL) and then the mixture was stirred at 70° C. for 3 h. The mixture was cooled to 0° C., diluted with THF (20 mL), quenched with Na₂SO₄·10H₂O (5.00 g), stirred at 20° C. for 30 min, and then filtered. The filtrate was concentrated in vacuo to give crude 2-(5-fluoro-4-methoxy-1H-indol-3-yl)-N-methylethan-1-amine (300 mg) as a brown oil which was used in the subsequent step without further purification.

### Step 3: N-ethyl-2-(5-fluoro-4-methoxy-1H-indol-3-yl)-N-methylethan-1-amine (P-92)

To an ice-cold (0° C.) solution of crude 2-(5-fluoro-4-methoxy-1H-indol-3-yl)-N-methylethan-1-amine (300 mg) in MeOH (3 mL) was added AcOH (324.2 mg, 5.40 mmol), NaBH₃CN (169.6 mg, 2.70 mmol), 40% w/w aqueous acetaldehyde (297 mg, 2.70 mmol) and the mixture was stirred at 20° C. for 16 h. The mixture was adjusted to pH 8 with saturated aqueous Na₂CO₃ solution and the organic solvent evaporated in vacuo. The residue was dissolved in H₂O (20 mL), extracted with EtOAc (80 mL×3), and the combined organic layers were washed with brine (20 mL), dried over Na₂SO₄, filtered and the filtrate concentrated in vacuo. The residue was purified by preparative HPLC (column: Waters Xbridge Prep OBD C18 150*40 mm*10 μm; mobile phase: [water (NH₄HCO₃)-ACN]; gradient: 12%-42% B over 15 min) to give N-ethyl-2-(5-fluoro-4-methoxy-1H-indol-3-yl)-N-methylethan-1-amine (20.6 mg, 2% over 3 steps) as a yellow solid. ¹H NMR (400 MHZ, MeOD-d₄): δ 7.01 (s, 1H), 6.97 (dd, J=8.8, 3.6 Hz, 1H), 6.86 (dd, J=12.4, 8.8 Hz, 1H), 4.01 (d, J=2.0 Hz, 3H), 3.07-2.96 (m, 2H), 2.79-2.70 (m, 2H), 2.61 (q, J=7.2 Hz, 2H), 2.39 (s, 3H), 1.15 (t, J=7.2 Hz, 3H). ¹⁹F NMR (376 MHZ, MeOD-d₄): δ −148.0. LCMS (ESI+): m/z 251.2 [M+H]⁺. HPLC Purity (254 nm): 96.8%.

### Example 65: N-(2-(5-fluoro-4-methoxy-1H-indol-3-yl)ethyl)-N-methylpropan-2-amine (P-93)

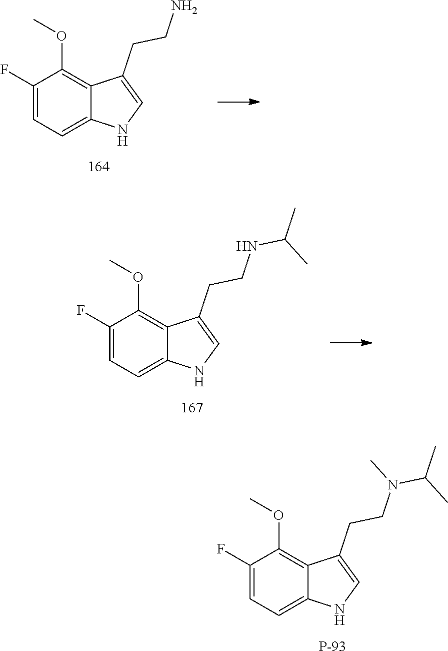

### Step 1: N-(2-(5-fluoro-4-methoxy-1H-indol-3-yl)ethyl)propan-2-amine (167)

To an ice-cold (0° C.) solution of 2-(5-fluoro-4-methoxy-1H-indol-3-yl)ethan-1-amine (800 mg, 3.84 mmol) in MeOH (8 mL) was added AcOH (923 mg, 15.4 mmol), NaBH₃CN (482.9 mg, 7.68 mmol), acetone (1.12 g, 19.2 mmol), and the mixture was stirred at 20° C. for 16 h. The mixture was adjusted to pH 8 with saturated aqueous Na₂CO₃ solution and the organic solvent evaporated in vacuo. The residue was dissolved in H₂O (20 mL), extracted with EtOAc (60 mL×3), and the combined organic phase was washed with brine (20 mL), dried over Na₂SO₄, filtered and the filtrate concentrated in vacuo. The residue was purified by preparative HPLC (column: Waters Xbridge Prep OBD C18 150×40 mm×10 μm; mobile phase: [water (NH₄HCO₃)-ACN]; B: 10-40%, 15 min) to give N-(2-(5-fluoro-4-methoxy-1H-indol-3-yl)ethyl)propan-2-amine (210 mg, 22% yield) as a yellow solid. ¹H NMR (400 MHZ, MeOD-d₄): δ 7.02 (s, 1H), 6.98 (dd, J=8.8, 3.6 Hz, 1H), 6.87 (dd, J=12.4, 8.8 Hz, 1H), 4.01 (d, J=2.0 Hz, 3H), 3.08-2.95 (m, 2H), 2.95-2.77 (m, 3H), 1.08 (d, J=6.4 Hz, 6H). ¹⁹F NMR (376 MHZ, MeOD-d₄): δ −147.9.

### Step 2: N-(2-(5-fluoro-4-methoxy-1H-indol-3-yl)ethyl)-N-methylpropan-2-amine (P-93)

To an ice-cold (0° C.) solution of N-(2-(5-fluoro-4-methoxy-1H-indol-3-yl)ethyl)propan-2-amine (210 mg, 0.838 mmol) in MeOH (2.10 mL) was added AcOH (202 mg, 3.36 mmol), NaBH₃CN (105 mg, 1.68 mmol), 37% w/w aqueous formaldehyde (126 mg, 1.56 mmol), and the mixture was stirred at 20° C. for 16 h. The mixture was adjusted to pH 8 with saturated aqueous Na₂CO₃ solution and the organic solvent evaporated in vacuo. The residue was dissolved in H₂O (20 mL), extracted with EtOAc (50 mL×3), and the combined organic layers were washed with brine (20 mL), dried over Na₂SO₄, filtered and the filtrate concentrated in vacuo. The residue was purified by preparative HPLC (column: Waters Xbridge 150×25 mm×5 μm; mobile phase: [water (NH₃ aq.)-ACN]; B: 40-70%, 10 min) to give N-(2-(5-fluoro-4-methoxy-1H-indol-3-yl)ethyl)-N-methylpropan-2-amine (32.1 mg, 15%) as a yellow solid. ¹H NMR (400 MHz, MeOD-d₄): δ 7.01 (s, 1H), 6.97 (dd, J=8.8, 3.6 Hz, 1H), 6.86 (dd, J=12.4, 8.8 Hz, 1H), 4.01 (d, J=2.0 Hz, 3H), 3.04-2.90 (m, 3H), 2.80-2.70 (m, 2H), 2.38 (s, 3H), 1.10 (d, J=6.8 Hz, 6H). ¹⁹F NMR (376 MHZ, MeOD-d₄): δ −148.1. LCMS (ESI+): m/z 265.2 [M+H]⁺. HPLC Purity (220 nm): 96.1%.

### Example 66: N,N-diethyl-2-(5-fluoro-4-methoxy-1H-indol-3-yl)ethan-1-amine (P-94)

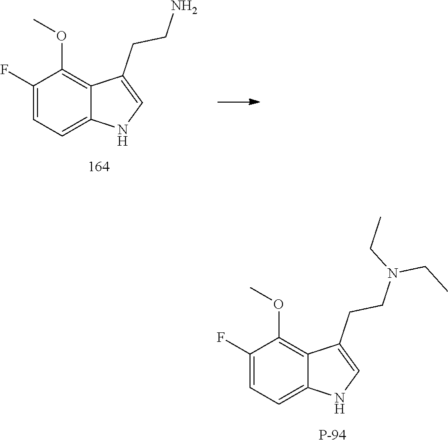

### Step 1: N,N-diethyl-2-(5-fluoro-4-methoxy-1H-indol-3-yl)ethan-1-amine (P-94)

To an ice-cold (0° C.) solution of 2-(5-fluoro-4-methoxy-1H-indol-3-yl)ethan-1-amine (500 mg, 2.40 mmol) in MeOH (5 mL) was added AcOH (577 mg, 9.60 mmol), NaBH₃CN (302 mg, 4.80 mmol), 40% w/w aqueous acetaldehyde (661 mg, 6.00 mmol), and the mixture was stirred at 20° C. for 3 h. The mixture was adjusted to pH 8 with saturated aqueous Na₂CO₃ and the organic solvent evaporated in vacuo. The residue was dissolved in H₂O (20 mL), extracted with EtOAc (80 mL×3), and the combined organic phase was washed with brine (20 mL), dried over Na₂SO₄, filtered and, the filtrate concentrated in vacuo. The residue was purified by preparative HPLC (column: Waters Xbridge Prep OBD C18 150×40 mm×10 μm; mobile phase: [water (NH₄HCO₃)-ACN]; B: 10-40%, 15 min) to give N,N-diethyl-2-(5-fluoro-4-methoxy-1H-indol-3-yl)ethan-1-amine (34.9 mg, 5.5%) as a yellow solid. ¹H NMR (400 MHZ, MeOD-d₄): δ 7.04-6.93 (m, 2H), 6.86 (dd, J=12.4, 8.8 Hz, 1H), 4.01 (d, J=2.0 Hz, 3H), 3.04-2.94 (m, 2H), 2.87-2.77 (m, 2H), 2.71 (q, J=7.2 Hz, 4H), 1.14 (t, J=7.2 Hz, 6H). ¹⁹F NMR (376 MHZ, MeOD-d₄): δ −148.0. LCMS (ESI+): m/z 265.1 [M+H]⁺ HPLC Purity (220 nm): 95.3%.

### Example 67: N-(2-(5-fluoro-4-methoxy-1H-indol-3-yl)ethyl)-N-isopropylpropan-2-amine (P-95)

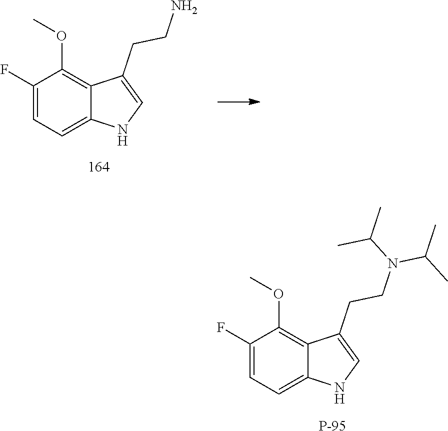

### Step 1: N-(2-(5-fluoro-4-methoxy-1H-indol-3-yl)ethyl)-N-isopropylpropan-2-amine (P-95)

To an ice-cold (0° C.) solution of 2-(5-fluoro-4-methoxy-1H-indol-3-yl)ethan-1-amine (800 mg, 3.84 mmol) in MeOH (8 mL) was added AcOH (923 mg, 15.4 mmol), NaBH₃CN (2.41 g, 38.4 mmol), acetone (2.23 g, 38.4 mmol), and the mixture was stirred at 20° C. for 3 h. The mixture was adjusted to pH 8 with saturated aqueous Na₂CO₃ and concentrated in vacuo. The residue was dissolved in H₂O (30 mL), extracted with EtOAc (100 mL×3), and the combined organic layers were washed with brine (30 mL), dried over Na₂SO₄, filtered, and the filtrate concentrated in vacuo. The residue was purified by preparative HPLC (column: Waters Xbridge 150×25 mm×5 μm; mobile phase: [water (NH₄HCO₃)-ACN]; B: 15-45%, 10 min) to give N-(2-(5-fluoro-4-methoxy-1H-indol-3-yl)ethyl)-N-isopropylpropan-2-amine (21.4 mg, 2%) as a yellow solid. ¹H NMR (400 MHZ, MeOD-d₄): δ 7.01 (s, 1H), 6.97 (dd, J=8.8, 3.6 Hz, 1H), 6.86 (dd, J=12.4, 8.8 Hz, 1H), 4.00 (d, J=1.6 Hz, 3H), 3.11-3.22 (m, 2H), 2.87-2.99 (m, 2H), 2.71-2.82 (m, 2H), 1.15 (d, J=6.4 Hz, 12H). ¹⁹F NMR (376 MHZ, MeOD-d₄): δ −147.6. LCMS (ESI+): m/z 293.2 [M+H]⁺. HPLC Purity (220 nm): 99.7%.

Scheme 15: Compounds of general formula (I) can be synthesised from the appropriately substituted indole following the outlined sequence of steps in Scheme 15 or similar as one skilled in the art may consider. Substituted indole cores could be condensed with dimethylamino-2-nitroethylene to give nitrovinyl indoles. Reduction of such indoles proved viable in accessing unsubstituted ethyl amine analogues that could subsequently undergo reductive alkylation to access compounds of general formula (I) (exemplified by P-14). One skilled in the art will recognise that protecting the amine with a suitable protecting group such as benzyl, followed by alkylation, subsequent deprotection and a second alkylation would allow access to differentially alkylated compounds of general formula (I) disclosed herein.

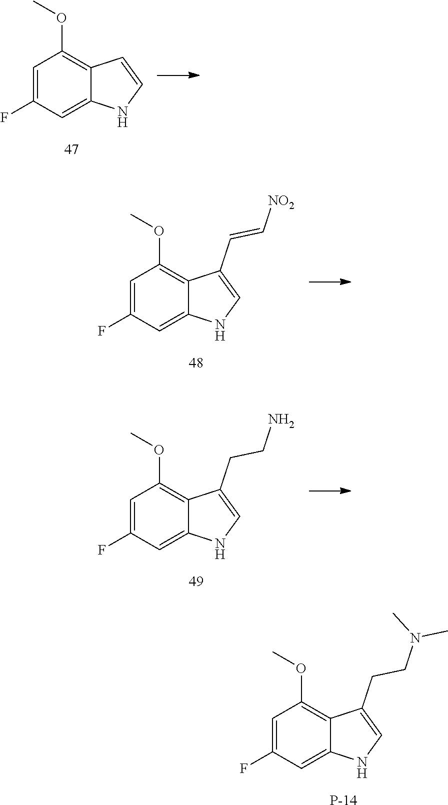

### Example 68: 2-(6-fluoro-4-methoxy-1H-indol-3-yl)-N,N-dimethylethan-1-amine (P-14)

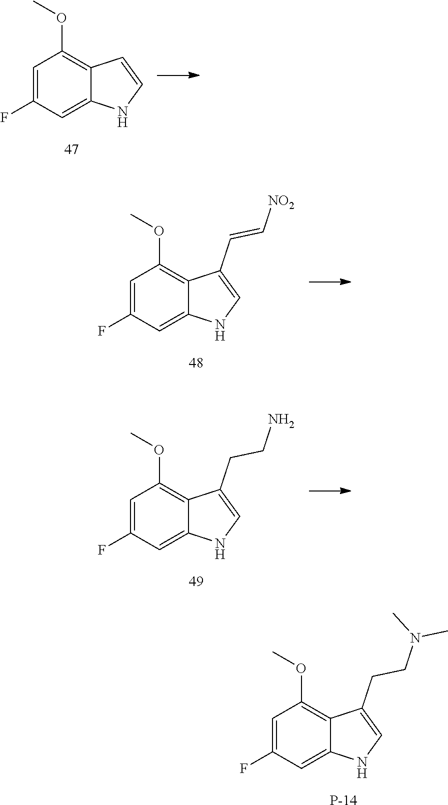

### Step 1: (E)-6-fluoro-4-methoxy-3-(2-nitrovinyl)-1H-indole (48)

To a solution of 6-fluoro-4-methoxy-1H-indole (300 mg, 1.82 mmol) in TFA (1.80 mL) was added (E)-N,N-dimethyl-2-nitroethen-1-amine (210 mg, 1.82 mmol) and the reaction mixture was stirred at 20° C. for 30 min and then adjusted to pH 8 with saturated aqueous NaHCO₃ solution and extracted with EtOAc (30 ml×2). The combined organic layers were washed with brine (20 mL), dried over Na₂SO₄, filtered, and the filtrate concentrated in vacuo. The residue was triturated with petroleum ether/EtOAc (20 mL, v/v, 10/1) at 20° C. for 30 min to give (E)-6-fluoro-4-methoxy-3-(2-nitrovinyl)-1H-indole (400 mg, 93%) as a red solid. ¹H NMR (400 MHZ, DMSO-d₆): δ 8.47 (d, J=13.2 Hz, 1H), 8.22 (s, 1H), 8.03 (d, J=13.2 Hz, 1H), 6.88 (dd, J=9.2, 2.0 Hz, 1H), 6.66 (dd, J=12.0, 2.0 Hz, 1H), 3.95 (s, 3H).

### Step 2: 2-(6-fluoro-4-methoxy-1H-indol-3-yl)ethan-1-amine (49)

A solution of (E)-6-fluoro-4-methoxy-3-(2-nitrovinyl)-1H-indole (300 mg, 1.27 mmol) in THF (5 mL) at 0° C. was treated with LiAlH₄ (530 mg, 13.9 mmol) in portions and the mixture was stirred at 80° C. for 3 h. The reaction mixture was cooled to 0° C., quenched with Na₂SO₄·10H₂O (3.0 g) and stirred at 20° C. for 30 min. The mixture was filtered, and the filter cake washed with THF (20 mL). The combined filtrates were concentrated in vacuo to afford crude 2-(6-fluoro-4-methoxy-1H-indol-3-yl)ethan-1-amine (170 mg) as a brown oil which was used in the subsequent step without further purification.

### Step 3: 2-(6-fluoro-4-methoxy-1H-indol-3-yl)-N,N-dimethylethan-1-amine (P-14)

To an ice-cold (0° C.) solution of 2-(6-fluoro-4-methoxy-1H-indol-3-yl)ethan-1-amine (0.17 g) in MeOH (5 mL) was added NaBH₃CN (102 mg, 1.63 mmol), AcOH (196 mg, 3.27 mmol) and 37% w/w aqueous formaldehyde solution (165 mg, 2.04 mmol). The reaction mixture was stirred at 20° C. for 3 h, and then the pH of the reaction mixture was adjusted to pH 8 with saturated aqueous Na₂CO₃ and the organic solvent evaporated in vacuo. The residue was dissolved in H₂O (30 mL) and extracted with EtOAc (10 mL×2). The combined organic layers were washed with brine (10 mL), dried over anhydrous Na₂SO₄, filtered, and the filtrate concentrated in vacuo. The residue was purified by preparative HPLC (column: Waters Xbridge Prep OBD C18 150×40 mm×10 μm; mobile phase: [water (NH₄HCO₃)-ACN]; B: 10-40%, 8 min) to give 2-(6-fluoro-4-methoxy-1H-indol-3-yl)-N,N-dimethylethan-1-amine (41.0 mg, 20%) as a yellow oil. ¹H NMR (400 MHZ, MeOD-d₄): δ 6.88 (s, 1H), 6.62 (dd, J=9.6, 2.0 Hz, 1H), 6.30 (dd, J=12.0, 2.0 Hz, 1H), 3.89 (s, 3H), 2.99-3.03 (m, 2H), 2.66-2.70 (m, 2H), 2.38 (s, 6H). ¹⁹F NMR (376 MHZ, MeOD-d₄): δ −122.2. LCMS (ESI+): m/z 237.1 [M+H]⁺. HPLC Purity (220 nm): 95.8%.

Scheme 16: Compounds of general formula (I) can be synthesised from the appropriately substituted indole in a similar manner to those outlined in Scheme 16. Glyoxylation of substituted indoles with oxalyl chloride followed by treatment with an appropriately substituted amine gave glyoxamide intermediates which when subjected to reductive conditions provides access to compounds of general formula (I) (exemplified by P-96). One skilled in the art will recognise that utilising differentially substituted amines would allow access to compounds of general formula (I) disclosed herein.

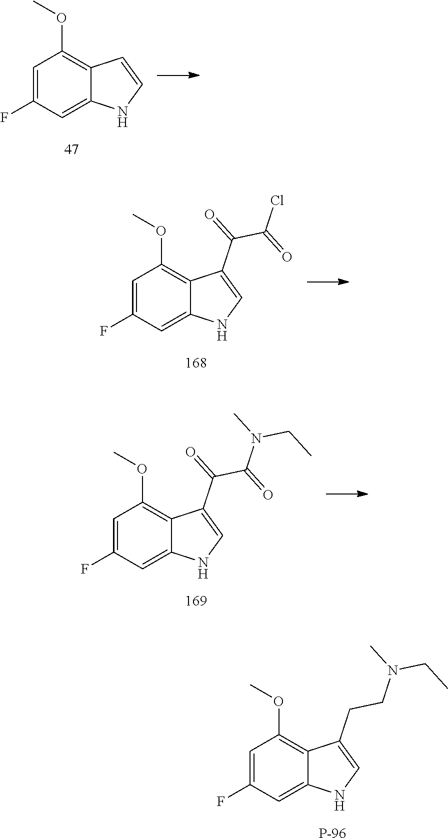

### Example 69: N-ethyl-2-(6-fluoro-4-methoxy-1H-indol-3-yl)-N-methylethan-1-amine (P-96)

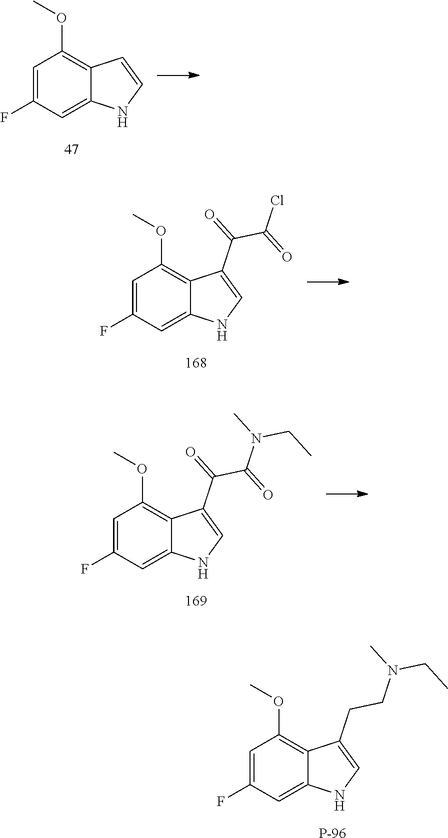

### Step 1: 2-(6-fluoro-4-methoxy-1H-indol-3-yl)-2-oxoacetyl chloride (168)

To an ice-cold solution of 6-fluoro-4-methoxy-1H-indole (1.0 g, 6.05 mmol) in anhydrous Et₂O (50 mL) was added dropwise a 2:1 solution of anhydrous Et₂O and oxalyl chloride (0.78 mL, 1.5 eq., 9.08 mmol), under N₂. The reaction was then stirred at ambient temperature for 16 h and the precipitate was collected by filtration under vacuum, washed with Et₂O (2×5 mL), and then dried under vacuum to yield 2-(6-fluoro-4-methoxy-1H-indol-3-yl)-2-oxoacetyl chloride (780 mg, 50%) as a yellow solid. ¹H NMR (400 MHZ, DMSO-d₆): δ 12.43 (s, 1H), 8.12 (d, J=2.8 Hz, 1H), 6.90 (dd, J=9.0, 2.4 Hz, 1H), 6.64 (dd, J=12.0, 2.4 Hz, 1H), 3.83 (s, 3H). ¹³C NMR (101 MHZ, DMSO-d₆): δ 183.0, 167.0, 160.3 (d, J=237.6 Hz), 154.1 (d, J=12.7 Hz), 137.6 (d, J=12.7 Hz), 135.3 (d, J=2.2 Hz), 113.2, 111.2 (d, J=0.9 Hz), 92.9 (d, J=28.8 Hz), 91.4 (d, J=26.3 Hz), 55.4.

### Step 2: N-ethyl-2-(6-fluoro-4-methoxy-1H-indol-3-yl)-N-methyl-2-oxoacetamide (169)

To a suspension of 2-(6-fluoro-4-methoxy-1H-indol-3-yl)-2-oxoacetyl chloride (350 mg, 1.37 mmol) in CH₂Cl₂ (10 mL) was added methylethylamine (0.29 mL, 2.5 equiv., 3.42 mmol) at 0° C. under N₂. The reaction was warmed to ambient temperature and stirred for 1 h before being diluted with CH₂Cl₂ (10 mL) and subsequently washed with 0.1 M aq. HCl (15 mL), H₂O (10 mL×3), brine (20 mL×1), and the organic layer was dried over Na₂SO₄, filtered, and the filtrate concentrated in vacuo. The oily residue was purified by crystallisation in CH₂Cl₂ and hexane to afford N-ethyl-2-(6-fluoro-4-methoxy-1H-indol-3-yl)-N-methyl-2-oxoacetamide as a white solid (350 mg, 92%). The product was a mixture of two rotamers in a ratio of approximately 1:3 (A:B). Rotamer A: ¹H NMR (400 MHZ, MeOD-d₄): δ 7.99 (s, 1H), 6.82 (dd, J=8.8, 2.0 Hz, 1H), 6.59 (dd, J=11.6, 2.0 Hz, 1H), 3.90 (s, 3H), 3.58 (q, J=7.2 Hz, 2H), 3.02 (s, 3H), 1.25 (t, J=7.2 Hz, 3H). Rotamer B: ¹H NMR (400 MHZ, MeOD-d₄): δ 8.03 (s, 1H), 6.82 (dd, J=8.8, 2.0 Hz, 1H), 6.59 (dd, J=11.6, 2.0 Hz, 1H), 3.90 (s, 3H), 3.39 (q, J=7.2 Hz, 2H), 3.08 (s, 3H), 1.19 (t, J=7.2 Hz, 3H).

### Step 3: N-ethyl-2-(6-fluoro-4-methoxy-1H-indol-3-yl)-N-methylethan-1-amine (P-96)

To ice-cold anhydrous THF (15 mL) was added LiAlH₄ (218 mg, 8 eq., 5.75 mmol) in portions. The resulting ice-cold suspension was then treated dropwise with N-ethyl-2-(6-fluoro-4-methoxy-1H-indol-3-yl)-N-methyl-2-oxoacetamide (0.2 g, 0.72 mmol), pre-dissolved in minimal anhydrous THF, at a rate which maintained a gentle reflux. The reaction mixture was then refluxed under N₂ for 16 h. The reaction was then cooled in an ice bath and quenched by sequential dropwise addition of cold H₂O (0.2 mL), 3.75 M aq. NaOH (0.2 mL), and H₂O (0.6 mL). The resulting suspension was then dried with Na₂SO₄ and filtered through a pad of celite. The filter cake was eluted with additional hot THF (20 mL×2), and the combined filtrate was concentrated in vacuo to afford N-ethyl-2-(6-fluoro-4-methoxy-1H-indol-3-yl)-N-methylethan-1-amine (170 mg, 95%) as a lightly coloured oil. ¹H NMR (400 MHZ, MeOD-d₄): δ 6.85 (s, 1H), 6.62 (dd, J=9.6, 2.0 Hz, 1H), 6.29 (dd, J=12.0, 2.0 Hz, 1H), 3.87 (s, 3H), 3.04-2.88 (m, 2H), 2.75-2.59 (m, 2H), 2.55 (q, J=7.2 Hz, 2H), 2.34 (s, 3H), 1.12 (t, J=7.2 Hz, 3H). ¹³C NMR (101 MHZ, MeOD-d₄): δ161.2 (d, J=235.1 Hz), 156.0 (d, J=12.6 Hz), 138.7 (d, J=15.4 Hz), 122.1 (d, J=3.2 Hz), 114.9, 114.4, 91.1 (d, J=26.2 Hz), 90.1 (d, J=29.7 Hz), 60.1, 55.6, 52.0, 41.8, 24.8, 11.9.

### Step 3a: N-ethyl-2-(6-fluoro-4-methoxy-1H-indol-3-yl)-N-methylethan-1-amine fumarate (P-96⋅fumarate)

To a solution of fumaric acid (88 mg, 0.76 mmol) in minimal refluxing acetone was added a solution of N-ethyl-2-(6-fluoro-4-methoxy-1H-indol-3-yl)-N-methylethan-1-amine (190 mg, 0.76 mmol) in minimal warm acetone. The resulting solution was allowed to cool to ambient temperature and stood overnight to afford N-ethyl-2-(6-fluoro-4-methoxy-1H-indol-3-yl)-N-methylethan-1-amine as the fumarate salt (178 mg, 76%) which were off-white crystals. ¹H NMR (400 MHZ, DMSO-d₆): δ 10.97 (s, 1H), 7.03 (d, J=2.4 Hz, 1H), 6.70 (dd, J=9.6, 2.0 Hz, 1H), 6.51 (s, 2H), 6.40 (dd, J=12.0, 2.0 Hz, 1H), 3.86 (s, 3H), 3.06-2.87 (m, 6H), 2.57 (s, 3H), 1.16 (t, J=7.2 Hz, 3H). ¹³C NMR (101 MHZ, DMSO-d₆): δ 167.7, 159.4 (d, J=233.3 Hz), 154.1 (d, J=12.7 Hz), 136.7 (d, J=15.4 Hz), 134.9, 122.2 (d, J=3.0 Hz), 113.2, 110.7, 90.4 (d, J=25.7 Hz), 89.4 (d, J=29.3 Hz), 56.3, 55.4, 49.7, 39.4, 21.8, 9.8. LCMS (ESI+): m/z 251.3 [M+H]⁺. qNMR Purity (ERETIC): 95.2%.

### Example 70: N-(2-(6-fluoro-4-methoxy-1H-indol-3-yl)ethyl)-N-methylpropan-2-amine (P-97)

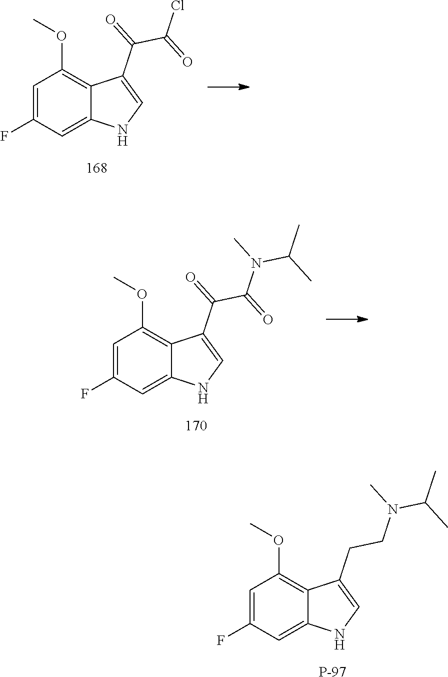

### Step 1: 2-(6-fluoro-4-methoxy-1H-indol-3-yl)-N-isopropyl-N-methyl-2-oxoacetamide (170)

To an ice-cold suspension of 2-(6-fluoro-4-methoxy-1H-indol-3-yl)-2-oxoacetyl chloride (250 mg, 0.98 mmol) in CH₂Cl₂ (10 mL) was added methyl(isopropyl)amine (255 μL, 2.5 eq., 2.44 mmol) under N₂. The reaction was warmed to ambient temperature and stirred for a further 1 h. The reaction was diluted with CH₂Cl₂ (10 mL) and subsequently washed with 0.1 M aq. HCl (15 mL), H₂O (10 mL×3), brine (20 mL×1). The organic layer was dried over Na₂SO₄, filtered, and the filtrate concentrated in vacuo. The residue was purified by flash chromatography (0.1% to 5% MeOH in DCM) to afford the 2-(6-fluoro-4-methoxy-1H-indol-3-yl)-N-isopropyl-N-methyl-2-oxoacetamide (270 mg, 94%) as an off-white solid. The product was a mixture of two rotamers in a ratio of approximately 1:2 (A:B). Rotamer A: ¹H NMR (400 MHZ, MeOD-d₄): δ 7.94 (s, 1H), 6.81 (m, 1H), 6.59 (dd, J=11.6, 1.6 Hz, 1H), 4.77 (m, 1H), 3.90 (m, 3H), 2.87 (s, 3H), 1.26 (d, J=6.8 Hz, 6H). Rotamer B: ¹H NMR (400 MHZ, MeOD-d₄): δ 8.05 (s, 1H), 6.81 (m, 1H), 6.59 (dd, J=11.6, 1.6 Hz, 1H), 4.00 (sept, J=6.8 Hz, 1H), 3.90 (m, 3H), 2.96 (s, 3H), 1.21 (d, J=6.8 Hz, 6H). Rotamer B: ¹³C NMR (101 MHZ, MeOD-d₄): δ 188.1, 170.5, 163.8 (d, J=239.7 Hz), 156.0 (d, J=12.1 Hz), 140.0 (d, J=15.5 Hz), 137.2, 115.8, 112.1, 94.4 (d, J=29.1 Hz), 92.5 (d, J=26.6 Hz), 56.4, 51.1, 25.5, 20.0.

### Step 2: N-(2-(6-fluoro-4-methoxy-1H-indol-3-yl)ethyl)-N-methylpropan-2-amine (P-97)

To ice-cold anhydrous THF (25 mL) was added LiAlH₄ (260 mg, 8 eq., 6.84 mmol) in portions. The resulting ice-cold suspension was then treated dropwise with 2-(6-fluoro-4-methoxy-1H-indol-3-yl)-N-isopropyl-N-methyl-2-oxoacetamide (250 mg, 0.86 mmol), pre-dissolved in minimal anhydrous THF, at a rate which maintained a gentle reflux. The reaction mixture was then refluxed under N₂ for 16 h. The reaction was then cooled in an ice bath and quenched by sequential dropwise addition of cold H₂O (0.3 mL), 3.75 M aq. NaOH (0.3 mL), and H₂O (0.9 mL). The resulting suspension was then dried with Na₂SO₄ and filtered through a pad of celite. The filter cake was eluted with additional hot THF (50 mL×2) and the combined filtrate was concentrated in vacuo to afford N-(2-(6-fluoro-4-methoxy-1H-indol-3-yl)ethyl)-N-methylpropan-2-amine (215 mg, 95%) as a lightly coloured oil. ¹H NMR (400 MHZ, MeOD-d₄): δ 6.86 (s, 1H), 6.62 (dd, J=9.6, 2.0 Hz, 1H), 6.29 (dd, J=12.0, 2.0 Hz, 1H), 3.88 (s, 3H), 3.07-2.81 (m, 3H), 2.74-2.61 (m, 2H), 2.33 (s, 3H), 1.08 (d, J=6.8 Hz, 6H). ¹³C NMR (101 MHZ, MeOD-d₄): δ 160.3 (d, J=234.2 Hz), 154.6 (d, J=12.7 Hz), 137.4 (d, J=15.2 Hz), 120.7 (d, J=3.3 Hz), 113.5, 113.1, 89.7 (d, J=26.1 Hz), 88.7 (d, J=29.5 Hz), 55.3, 54.3, 53.2, 36.4, 24.3, 16.8.

### Step 2a: N-(2-(6-fluoro-4-methoxy-1H-indol-3-yl)ethyl)-N-methylpropan-2-amine fumarate (P-97⋅fumarate)

To a solution of fumaric acid (83 mg, 0.72 mmol) in minimal refluxing acetone was added a solution of N-(2-(6-fluoro-4-methoxy-1H-indol-3-yl)ethyl)-N-methylpropan-2-amine (190 mg, 0.72 mmol) in minimal warm acetone. The resulting solution was allowed to cool to RT and stood overnight to afford N-(2-(6-fluoro-4-methoxy-1H-indol-3-yl)ethyl)-N-methylpropan-2-amine as the fumarate salt (69 mg, 30%) which were off-white crystals. ¹H NMR (400 MHZ, DMSO-d₆): δ 10.96 (s, 1H), 7.02 (d, J=2.0 Hz, 1H), 6.70 (dd, J=9.6, 2.0 Hz, 1H), 6.51 (s, 2H), 6.39 (dd, J=12.0, 2.0 Hz, 1H), 3.86 (s, 3H), 3.30 (sept, J=6.4 Hz, 1H), 3.12-2.88 (m, 4H), 2.53 (s, 3H), 1.14 (d, J=6.4 Hz, 6H). ¹³C NMR (100 MHZ, DMSO-d₆): δ 167.5, 159.4 (d, J=233.3 Hz), 154.0, 136.6 (d, J=15.7 Hz), 134.9, 122.2, 113.2, 110.8, 90.4 (d, J=25.7 Hz), 89.4 (d, J=29.3 Hz), 55.4, 54.4, 53.8, 35.7, 22.8, 16.5. LCMS (ESI+): m/z 265.3 [M+H]⁺. qNMR Purity (ERETIC): 99.9%.

### Example 71: N,N-diethyl-2-(6-fluoro-4-methoxy-1H-indol-3-yl)ethan-1-amine (P-98)

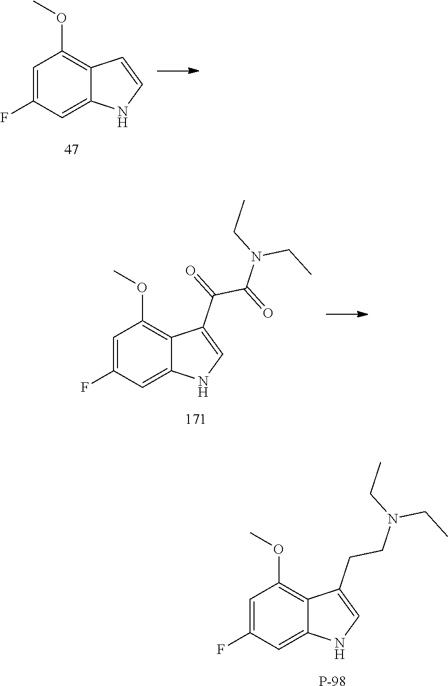

### Step 1: N,N-diethyl-2-(6-fluoro-4-methoxy-1H-indol-3-yl)-2-oxoacetamide (171)

To an ice-cold solution of 6-fluoro-4-methoxy-1H-indole (0.2 g, 1.21 mmol) in anhydrous Et₂O (50 mL) was added dropwise a 1:1 solution of anhydrous Et₂O and (COCl)₂ (125 μL, 1.2 eq., 1.45 mmol), under N₂. The resulting solution was stirred at ambient temperature for 16 h. The reaction was cooled in an ice bath and treated with diethylamine, diluted 1:1 with anhydrous Et₂O, was added over 1 hour until the pH of the reaction solution was basic, which required a total of 5 equivalents (0.63 mL, 6.05 mmol). The reaction was stirred at ambient temperature for 1 h and then concentrated under a flow of N₂. The residue was taken up in EtOAc (50 mL) and washed with H₂O (20 mL×3), and then brine (20 mL). The organic layer was concentrated, and the residue was purified by crystallisation in CH₂Cl₂ and hexane to afford N,N-diethyl-2-(6-fluoro-4-methoxy-1H-indol-3-yl)-2-oxoacetamide (155 mg, 44%) as a pink solid. ¹H NMR (400 MHZ, MeOD-d₄): δ 7.98 (s, 1H), 6.82 (dd, J=8.8, 2.0 Hz, 1H), 6.59 (dd, J=11.6, 2.0 Hz, 1H), 3.90 (s, 3H), 3.56 (q, J=7.2 Hz, 2H), 3.40 (q, J=7.2 Hz, 2H), 1.27 (t, J=7.2 Hz, 3H), 1.18 (t, J=7.2 Hz, 3H). ¹³C NMR (101 MHZ, MeOD-d₄): δ 188.0, 170.5, 162.7 (d, J=239.7 Hz), 156.0 (d, J=12.4 Hz), 139.8 (d, J=15.2 Hz), 137.2, 115.8, 112.2, 94.2 (d, J=29.1 Hz), 92.2 (d, J=26.7 Hz), 56.3, 44.0, 40.4, 14.2, 13.1.

### Step 2: N, N-diethyl-2-(6-fluoro-4-methoxy-1H-indol-3-yl)ethan-1-amine (P-98)

To ice-cold anhydrous THF (30 mL) was added LiAlH₄ (436 mg, 8 eq., 11.5 mmol) in portions. The resulting ice-cold suspension was then treated dropwise with N,N-diethyl-2-(6-fluoro-4-methoxy-1H-indol-3-yl)-2-oxoacetamide (420 mg, 1.44 mmol), pre-dissolved in minimal anhydrous THF, at a rate which maintained a gentle reflux. The reaction mixture was refluxed under N₂ for 16 h. The reaction mixture was then cooled in an ice bath and quenched by sequential dropwise addition of cold H₂O (0.4 mL), 3.75 M aqueous NaOH (0.4 mL), and H₂O (1.2 mL). The resulting suspension was then dried with Na₂SO₄ and filtered through a pad of celite. The filter cake was eluted with additional hot THF (50 mL×2) and the combined filtrate was concentrated in vacuo to afford N,N-diethyl-2-(6-fluoro-4-methoxy-1H-indol-3-yl)ethan-1-amine (330 mg, 87%) as a lightly coloured oil. ¹H NMR (400 MHZ, DMSO-d₆): δ 10.80 (s, 1H), 6.95 (d, J=1.6 Hz, 1H), 6.68 (dd, J=9.6, 2.0 Hz, 1H), 6.36 (dd, J=12.0, 2.0 Hz, 1H), 3.84 (s, 3H), 2.88-2.75 (m, 2H), 2.68-2.57 (m, 2H), 2.53 (m, 4H), 1.00 (t, J=7.2 Hz, 6H). ¹³C NMR (100 MHZ, DMSO-d₆): δ 159.3 (d, J=233.7 Hz), 154.3 (d, J=12.8 Hz), 136.7 (d, J=15.5 Hz), 121.5 (d, J=3.3 Hz), 113.5, 113.3, 90.2 (d, J=25.7 Hz), 89.2 (d, J=29.2 Hz), 55.4, 54.6, 46.5, 23.8, 12.1.

### Step 2a: N,N-diethyl-2-(6-fluoro-4-methoxy-1H-indol-3-yl)ethan-1-amine fumarate (P-98⋅fumarate)

To a solution of fumaric acid (132 mg, 1.13 mmol) in minimal refluxing acetone was added a solution of N,N-diethyl-2-(6-fluoro-4-methoxy-1H-indol-3-yl)ethan-1-amine (0.3 g, 1.13 mmol) in minimal warm acetone. The resulting solution was allowed to cool to ambient temperature and stood overnight to afford N,N-diethyl-2-(6-fluoro-4-methoxy-1H-indol-3-yl)ethan-1-amine as the fumarate salt (349 mg, 95%) which were off-white crystals. ¹H NMR (400 MHZ, DMSO-d₆): δ 10.98 (s, 1H), 7.04 (d, J=2.4 Hz, 1H), 6.71 (dd, J=9.6, 2.0 Hz, 1H), 6.50 (s, 2H), 6.40 (dd, J=12.0, 2.0 Hz, 1H), 3.86 (s, 3H), 3.06-2.85 (m, 8H), 1.16 (t, J=7.2 Hz, 6H). ¹³C NMR (101 MHz, DMSO-d₆): δ 167.6, 159.4 (d, J=232.9 Hz), 154.0 (d, J=12.8 Hz), 136.7 (d, J=15.3 Hz), 135.0, 122.4, 113.2, 110.7, 90.4 (d, J=25.9 Hz), 89.4 (d, J=29.0 Hz), 55.4, 52.5, 46.0, 21.7, 9.5. LCMS (ESI+): m/z 265.3 [M+H]⁺. qNMR Purity (ERETIC): 96.8%.

### Example 72: N-ethyl-N-(2-(6-fluoro-4-methoxy-1H-indol-3-yl)ethyl)propan-2-amine (P-99)

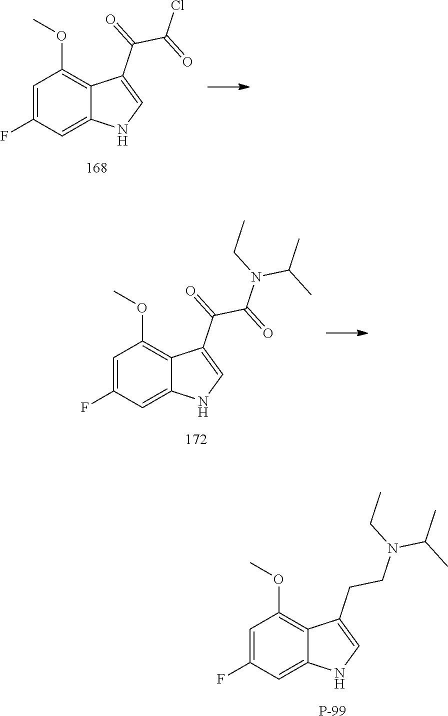

### Step 1: N-ethyl-2-(6-fluoro-4-methoxy-1H-indol-3-yl)-N-isopropyl-2-oxoacetamide (172)

To an ice-cold suspension of 2-(6-fluoro-4-methoxy-1H-indol-3-yl)-2-oxoacetyl chloride (250 mg, 0.98 mmol) in CH₂Cl₂ (10 mL) was treated with ethyl(isopropyl)amine (0.30 mL, 2.44 mmol) under N₂. The pH of the resulting solution was basic immediately after addition. The reaction was stirred at ambient temperature for 1 h and then diluted with CH₂Cl₂ (10 mL), washed with 0.1 M aq. HCl (15 mL), H₂O (10 mL×3), and brine (20 mL). The organic layer was dried over anhydrous Na₂SO₄, filtered, and the filtrate concentrated in vacuo. The residue was purified by crystallisation in CH₂Cl₂ and hexane to afford N-ethyl-2-(6-fluoro-4-methoxy-1H-indol-3-yl)-N-isopropyl-2-oxoacetamide (280 mg, 93%) as an off-white solid. The product was a mixture of two rotamers in a ratio of approximately 1:3 (A:B). Rotamer A: 1H NMR (400 MHZ, MeOD-d₄): δ 7.94 (s, 1H), 6.87-6.75 (m, 1H), 6.61-6.57 (dd, J=11.6, 1.6 Hz, 1H), 4.49 (sept, J=6.8 Hz, 1H), 3.90 (m, 3H), 3.37 (q, J=7.2 Hz, 1H), 1.36 (d, J=6.8 Hz, 6H), 1.17 (t, J=7.2 Hz, 3H). Rotamer B: ¹H NMR (400 MHZ, MeOD-d₄): δ 7.99 (s, 1H), 6.87-6.75 (m, 1H), 6.61-6.57 (dd, J=11.6, 1.6 Hz, 1H), 3.99 (sept, J=6.8 Hz, 1H), 3.90 (m, 3H), 3.47 (q, J=7.2 Hz, 1H), 1.31 (t, J=7.2 Hz, 3H), 1.22 (d, J=6.8 Hz, 6H). ¹³C NMR (101 MHZ, MeOD-d₄): δ 188.0, 170.4, 163.9 (d, J=240.0 Hz), 156.1 (d, J=12.5 Hz), 140.1 (d, J=15.5 Hz), 137.5, 115.7, 112.1, 94.4 (d, J=29.1 Hz), 92.4 (d, J=26.7 Hz), 56.3, 51.6, 36.2, 20.9, 14.9.

### Step 2: N-ethyl-N-(2-(6-fluoro-4-methoxy-1H-indol-3-yl)ethyl)propan-2-amine (P-99)

To ice-cold anhydrous THF (25 mL) was added LiAlH₄ (238 mg, 8 eq., 6.27 mmol) in portions. The resulting ice-cold suspension was then treated dropwise with N-ethyl-2-(6-fluoro-4-methoxy-1H-indol-3-yl)-N-isopropyl-2-oxoacetamide (240 mg, 0.78 mmol), pre-dissolved in minimal anhydrous THF, at a rate which maintained a gentle reflux. The reaction mixture was refluxed under N₂ for 16 h. The reaction mixture was then cooled in an ice bath and quenched by sequential dropwise addition of cold H₂O (0.3 mL), 3.75M aq. NaOH (0.3 mL), and H₂O (0.9 mL). The resulting suspension was then dried over Na₂SO₄ and filtered through a pad of celite. The filter cake was eluted with additional hot THF (50 mL×2) and the combined filtrate was concentrated in vacuo to afford N-ethyl-N-(2-(6-fluoro-4-methoxy-1H-indol-3-yl)ethyl)propan-2-amine (210 mg, 96%) as a lightly coloured oil. ¹H NMR (400 MHZ, MeOD-d₄): δ 6.85 (s, 1H), 6.62 (dd, J=9.6, 2.0 Hz, 1H), 6.29 (dd, J=12.0, 2.0 Hz, 1H), 3.88 (s, 3H), 3.07 (sept, J=6.4 Hz, 1H), 2.99-2.87 (m, 2H), 2.78-2.59 (m, 4H), 1.22-1.03 (m, 9H). ¹³C NMR (101 MHZ, MeOD-d₄): δ 161.6 (d, J=234.1 Hz), 155.9 (d, J=12.6 Hz), 138.8 (d, J=15.3 Hz), 122.1 (d, J=3.3 Hz), 114.1, 114.0, 91.1 (d, J=26.1 Hz), 90.1 (d, J=29.5 Hz), 55.7, 53.0, 51.6, 45.2, 26.6, 18.6, 13.1.

### Step 2a: N-ethyl-N-(2-(6-fluoro-4-methoxy-1H-indol-3-yl)ethyl)propan-2-amine fumarate (P-99-fumarate)

To a solution of fumaric acid (83 mg, 0.72 mmol) in minimal refluxing acetone was added a solution of N-ethyl-N-(2-(6-fluoro-4-methoxy-1H-indol-3-yl)ethyl)propan-2-amine (0.2 g, 0.72 mmol) in minimal warm acetone. The resulting solution was allowed to cool to RT and stood overnight to afford N-ethyl-N-(2-(6-fluoro-4-methoxy-1H-indol-3-yl)ethyl)propan-2-amine as the fumarate salt (211 mg, 87%) which were colourless crystals. ¹H NMR (400 MHZ, DMSO-d₆): δ 10.97 (s, 1H), 7.04 (d, J=2.0 Hz, 1H), 6.70 (dd, J=9.6, 2.0 Hz, 1H), 6.51 (s, 2H), 6.39 (dd, J=12.0, 2.0 Hz, 1H), 3.86 (s, 3H), 3.39 (sept, J=6.4 Hz, 1H), 3.12-2.75 (m, 6H), 1.17 (m, 9H). ¹³C NMR (100 MHZ, DMSO-d₆): δ 167.3, 159.4 (d, J=233.3 Hz), 154.0 (d, J=12.8 Hz), 136.7 (d, J=15.4 Hz), 135.0, 122.3, 113.2, 111.0, 90.4 (d, J=25.3 Hz), 89.4 (d, J=29.3 Hz), 55.4, 51.8, 50.7, 44.2, 23.9, 17.0, 11.4. LCMS (ESI+): m/z 279.3 [M+H]⁺. qNMR Purity (ERETIC): 100%.

### Example 73: N-ethyl-N-(2-(6-fluoro-4-methoxy-1H-indol-3-yl)ethyl)propan-2-amine (P-100)

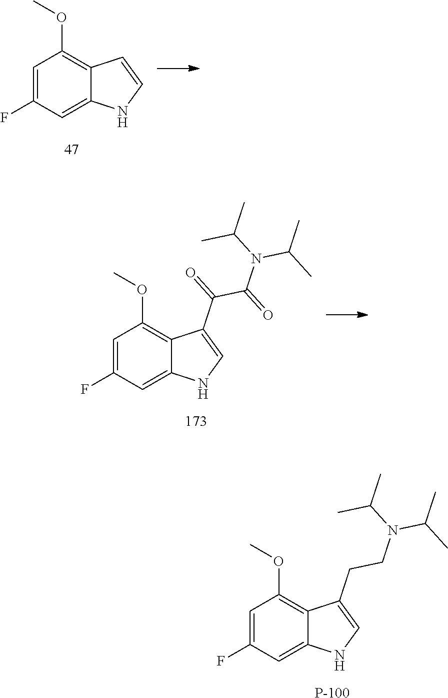

### Step 1: 2-(6-fluoro-4-methoxy-1H-indol-3-yl)-N,N-diisopropyl-2-oxoacetamide (173)

To an ice-cold solution of 6-fluoro-4-methoxy-1H-indole (0.5 g, 3.03 mmol) in anhydrous Et₂O (50 mL) was added dropwise a 1:1 solution of anhydrous Et₂O and (COCl)₂ (0.39 mL, 4.54 mmol), under N₂. The resulting solution was stirred at ambient temperature for 16 h. The reaction was cooled in an ice bath and treated with a 1:1 solution of anhydrous Et₂O and N,N-diisopropylamine (2.56 mL, 18.2 mmol). The reaction was stirred at ambient temperature for 1 h and then concentrated under a stream of N₂ gas. The residue was taken up in EtOAc (100 mL) and washed subsequently with 0.5 M aq. HCl, (20 mL×3), H₂O (20 mL), and finally brine (50 mL) before being dried over MgSO₄, filtered and the filtrate concentrated under reduced pressure. The resultant residue was purified by flash chromatography (0.1% to 5% MeOH in CH₂Cl₂) to afford 2-(6-fluoro-4-methoxy-1H-indol-3-yl)-N,N-diisopropyl-2-oxoacetamide (539 mg, 56%) as an off-white solid. ¹H NMR (400 MHZ, DMSO-d₆): δ 12.13 (s, 1H), 7.87 (d, J=2.4 Hz, 1H), 6.90 (dd, J=8.8, 2.0 Hz, 1H), 6.64 (dd, J=12.0, 2.0 Hz, 1H), 3.85 (s, 3H), 3.72 (sept, J=6.4 Hz, 1H), 3.57 (sept, J=6.8 Hz, 1H), 1.44 (d, J=6.8 Hz, 6H), 1.11 (d, J=6.4 Hz, 6H). ¹³C NMR (100 MHz, DMSO-d₆): δ 184.9, 167.9, 161.6 (d, J=237.8 Hz), 154.7 (d, J=12.5 Hz), 138.3 (d, J=15.6 Hz), 136.4, 114.0, 110.7, 93.1 (d, J=28.7 Hz), 91.3 (d, J=26.1 Hz), 55.8, 49.8, 44.6, 20.2, 20.0.

### Step 2: N-(2-(6-fluoro-4-methoxy-1H-indol-3-yl)ethyl)-N-isopropylpropan-2-amine (P-100)

To ice-cold anhydrous THF (40 mL) was added LiAlH₄ (379 mg, 8 eq., 9.99 mmol) in portions. The resulting ice-cold suspension was then treated dropwise with 2-(6-fluoro-4-methoxy-1H-indol-3-yl)-N,N-diisopropyl-2-oxoacetamide (0.4 g, 1.25 mmol), pre-dissolved in minimal anhydrous THF, at a rate which maintained a gentle reflux. The reaction mixture was refluxed under N₂ for 16 hours. The reaction mixture was then cooled in an ice bath and quenched by sequential dropwise addition of cold H₂O (0.4 mL), 3.75M aq. NaOH (0.4 mL), and H₂O (1.2 mL). The resulting suspension was then dried with Na₂SO₄ and filtered through a pad of celite. The filter cake was eluted with additional hot THF (50 mL×2) and the combined filtrate was concentrated in vacuo to afford N-(2-(6-fluoro-4-methoxy-1H-indol-3-yl)ethyl)-N-isopropylpropan-2-amine (360 mg, quant.) as a lightly coloured oil. ¹H NMR (400 MHZ, MeOD-d₄): δ 6.84 (s, 1H), 6.62 (dd, J=9.6, 2.0 Hz, 1H), 6.29 (dd, J=12.0, 2.0 Hz, 1H), 3.87 (s, 3H), 3.17-3.01 (m, 2H), 2.97-2.82 (m, 2H), 2.75-2.53 (m, 2H), 1.11 (d, J=6.4 Hz, 12H). ¹³C NMR (101 MHZ, MeOD-d₄): δ 161.2 (d, J=234.1 Hz), 155.8 (d, J=12.6 Hz), 138.7 (d, J=15.2 Hz), 121.9 (d, J=3.3 Hz), 114.9, 114.8, 91.1 (d, J=26.0 Hz), 90.0 (d, J=29.5 Hz), 55.6, 50.9, 50.2, 30.3, 20.5.

### Step 2a: N-(2-(6-fluoro-4-methoxy-1H-indol-3-yl)ethyl)-N-isopropylpropan-2-amine fumarate (P-100-fumarate)

To a solution of fumaric acid (103 mg, 0.89 mmol) in minimal refluxing acetone was added a solution of N-(2-(6-fluoro-4-methoxy-1H-indol-3-yl)ethyl)-N-isopropylpropan-2-amine (260 mg, 0.89 mmol) in minimal warm acetone. The resulting solution was allowed to cool to ambient temp and stood overnight to afford N-(2-(6-fluoro-4-methoxy-1H-indol-3-yl)ethyl)-N-isopropylpropan-2-amine as the fumarate salt (307 mg, quant.) which were colourless crystals. ¹H NMR (400 MHZ, DMSO-d₆): δ 11.01 (s, 1H), 7.07 (d, J=2.4 Hz, 1H), 6.71 (dd, J=9.6, 2.0 Hz, 1H), 6.52 (s, 2H), 6.40 (dd, J=12.0, 2.0 Hz, 1H), 3.86 (s, 3H), 3.42 (sept, J=6.4 Hz, 2H), 3.07-2.83 (m, 4H), 1.20 (d, J=6.4 Hz, 12H). ¹³C NMR (100 MHZ, DMSO-d₆): δ 167.3, 159.3 (d, J=233.3 Hz), 154.0 (d, J=12.8 Hz), 136.7 (d, J=15.5 Hz), 134.9, 122.4, 113.2, 111.0, 90.4 (d, J=25.5 Hz), 89.4 (d, J=29.1 Hz), 55.4, 51.5, 48.1, 18.6. LCMS (ESI+): m/z 293.2 [M+H]⁺. qNMR Purity (ERETIC): 100%.

Scheme 17: Compounds of general formula (I) can be synthesised from the appropriately substituted benzaldehyde following a sequence of synthetic steps outlined in Scheme 14 or similar as one skilled in the art may consider. Initial Knovenagel condensation of substituted benzaldehydes such as 174 with ethyl azidoacetate gives rise to vinyl azide 175 which can undergo a thermally induced intramolecular cyclisation to furnish indole 176. Standard ester hydrolysis and subsequent thermal decarboxylation gave indole 178. Such indole cores could then be condensed with dimethylamino-2-nitroethylene to give nitrovinyl indoles. Reduction of such indoles proved viable in accessing unsubstituted ethyl amine analogues that could subsequently undergo reductive alkylation to access compounds of general formula (I) (exemplified by P-101). One skilled in the art will recognise that intermediate 180 can be condensed with boc-anhydride then subjected to reductive conditions generating mono-methyl intermediate 182 that can undergo subsequent reductive alkylations with various aldehydes and ketones providing access to differentially alkylated compounds of general formula (I, exemplified by P-102) disclosed herein.

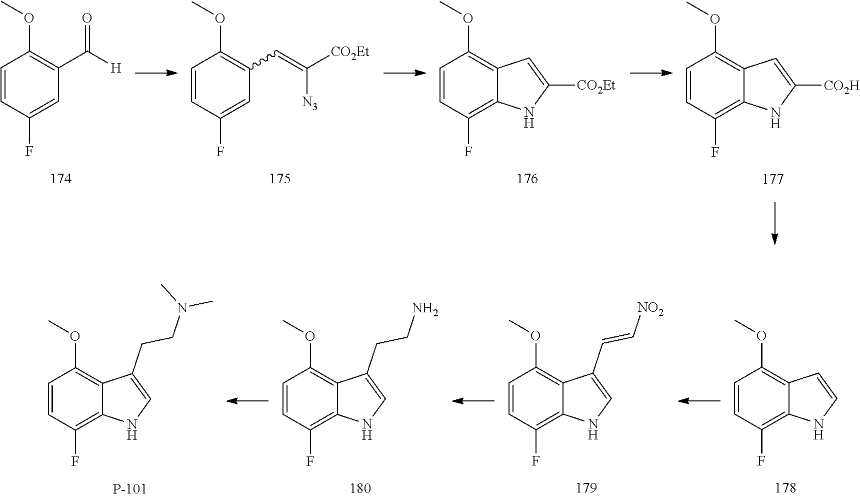

### Example 74: 2-(7-fluoro-4-methoxy-1H-indol-3-yl)-N,N-dimethylethan-1-amine (Example P-101)

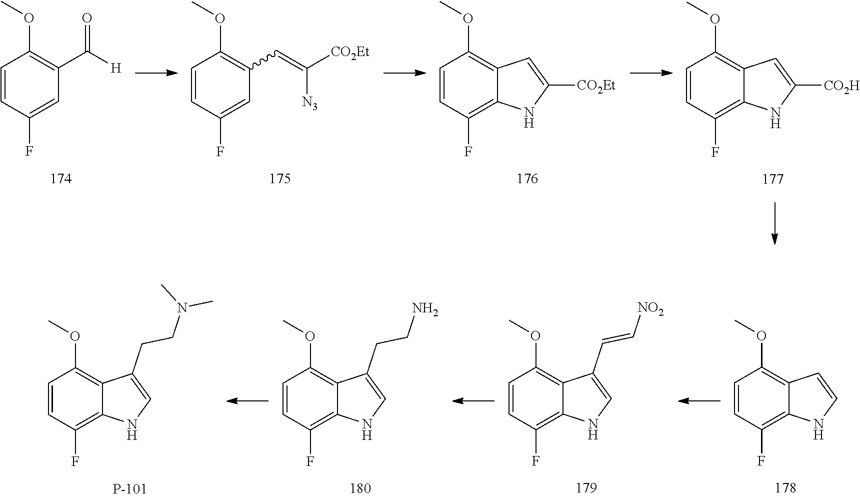

### Step 1: ethyl 2-azido-3-(5-fluoro-2-methoxyphenyl)acrylate (175)

A solution of 5-fluoro-2-methoxybenzaldehyde (5 g, 32.4 mmol) and ethyl 2-azidoacetate (12.5 g, 97.3 mmol) in EtOH (35 mL) at −20° C. was treated with a solution of 22% w/v NaOEt (30.1 g, 97.3 mmol) at −20° C. and then stirred at 0° C. for 2 h. The reaction mixture was poured into saturated aqueous NH₄Cl solution (100 mL), extracted with EtOAc (50 mL×2), and the combined organic layers were washed with brine (30 mL), dried over anhydrous Na₂SO₄, filtered, and concentrated in vacuo. The residue was purified by column chromatography (SiO₂, petroleum ether/EtOAc, v/v, 50/1 to 20/1) to afford crude ethyl 2-azido-3-(5-fluoro-2-methoxyphenyl) prop-2-enoate (8.0 g) as a light yellow solid which was used in the next step without further purification.

### Step 2: ethyl 7-fluoro-4-methoxy-1H-indole-2-carboxylate (176)

A solution of crude ethyl 2-azido-3-(5-fluoro-2-methoxyphenyl) prop-2-enoate (8.0 g) in xylene (48 mL) was stirred at 130° C. for 1 h. The reaction mixture was concentrated in vacuo, and the residue was purified by column chromatography (SiO₂, petroleum ether/EtOAc, v/v, 50/1 to 20/1) to afford ethyl 7-fluoro-4-methoxy-1H-indole-2-carboxylate (2.1 g, 27% over 2 steps) as a white solid. ¹H NMR (400 MHZ, CDCl₃): δ 8.94 (br. s, 1H), 7.36-7.34 (m, 1H), 6.93 (dd, J=10.4, 8.4 Hz, 1H), 6.35 (dd, J=8.4, 2.8 Hz, 1H), 4.41 (q, J=7.2 Hz, 2H), 3.94 (s, 3H), 1.42 (t, J=7.2 Hz, 3H).

### Step 3: 7-fluoro-4-methoxy-1H-indole-2-carboxylic acid (177)

A solution of ethyl 7-fluoro-4-methoxy-1H-indole-2-carboxylate (1.70 g, 7.17 mmol) in EtOH (6.80 mL) and H₂O (1.70 mL) was treated with KOH (1.01 g, 17.9 mmol) and the mixture was stirred at 60° C. for 4 h. The reaction was then cooled to ambient temperature, and the organic solvent was removed in vacuo. The residue was diluted with H₂O (20 mL), and the pH was adjusted to 3 using 1 M aqueous HCl. The acidified aqueous layer was extracted with EtOAc (30 mL×2) and the combined organic layer was washed with brine (20 mL), dried over anhydrous Na₂SO₄, filtered, and the filtrate concentrated in vacuo to afford 7-fluoro-4-methoxy-1H-indole-2-carboxylic acid (1.48 g, 99%) as white solid. ¹H NMR (400 MHZ, MeOD-d₄): δ 7.21 (d, J=2.8 Hz, 1H), 6.88 (dd, J=10.8, 8.4 Hz, 1H), 6.39 (dd, J=8.4, 2.8 Hz, 1H), 3.91 (s, 3H).

### Step 4: 7-fluoro-4-methoxy-1H-indole (178)

A solution of 7-fluoro-4-methoxy-1H-indole-2-carboxylic acid (1.48 g, 7.08 mmol) in quinoline (15 mL) was treated with copper (315 mg, 4.95 mmol) and the mixture was stirred at 230° C. for 2 h under an atmosphere of N₂. The reaction mixture was cooled to ambient temperature, diluted with EtOAc (60 mL), and washed sequentially with 1 M aqueous HCl (30 mL×3), saturated aqueous NaHCO₃ (30 mL×2), brine (20 mL), and then dried over anhydrous Na₂SO₄, filtered, and the filtrate concentrated in vacuo. The residue was purified by column chromatography (SiO₂, petroleum ether/EtOAc, v/v, 100/1 to 60/1) to afford 7-fluoro-4-methoxy-1H-indole (1.00 g, 86%) as a yellow oil. ¹H NMR (400 MHZ, MeOD-d₄): δ 8.32 (br s, 1H), 7.18-7.14 (m, 1H), 6.81 (dd, J=10.8, 8.4 Hz, 1H), 6.71-6.66 (m, 1H), 6.37 (dd, J=8.4, 3.2 Hz, 1H), 3.94 (s, 3H).

### Step 5: 7-fluoro-4-methoxy-3-(2-nitrovinyl)-1H-indole (179)

A solution of 7-fluoro-4-methoxy-1H-indole (600 mg, 3.63 mmol) in TFA (3.6 mL) was treated with (E)-N,N-dimethyl-2-nitroethen-1-amine (421 mg, 3.63 mmol) and the mixture was stirred at 20° C. for 1 h. The pH of the reaction mixture was adjusted to 8 with saturated aqueous NaHCO₃ and then extracted with EtOAc (30 mL×2). The combined organic layer was washed with brine (10 mL), dried over anhydrous Na₂SO₄, filtered, and the filtrate concentrated in vacuo. The residue was purified by column chromatography (SiO₂, petroleum ether/EtOAc, v/v, 50/1 to 10/1) to afford crude 7-fluoro-4-methoxy-3-(2-nitrovinyl)-1H-indole (350 mg) which was used in the subsequent step without further purification.

### Step 6: 2-(7-fluoro-4-methoxy-1H-indol-3-yl)ethan-1-amine (180)

To an ice-cold (0° C.) solution of crude 7-fluoro-4-methoxy-3-(2-nitrovinyl)-1H-indole (350 mg) in anhydrous THF (7 mL) was added LiAlH₄ (618 mg, 16.3 mmol) in portions over 15 min and then the mixture was stirred at 80° C. for 2 h. The reaction mixture was then cooled to 0° C., diluted with THF (50 mL), quenched with Na₂SO₄·10H₂O (3.0 g), and stirred at 20° C. for 30 min. The mixture was filtered, and the filtrate was concentrated in vacuo to afford 2-(7-fluoro-4-methoxy-1H-indol-3-yl)ethan-1-amine (300 mg, 40% over 2 steps) as a brown oil. ¹H NMR (400 MHZ, MeOD-d₄): δ 6.96 (s, 1H), 6.69 (dd, J=10.8, 8.4 Hz, 1H), 6.31 (dd, J=8.4, 3.2 Hz, 1H), 3.87 (s, 3H), 3.01-2.95 (m, 2H), 2.93-2.88 (m, 2H). LCMS (ESI+): m/z 209.1 [M+H]⁺.

### Step 7: 2-(7-fluoro-4-methoxy-1H-indol-3-yl)-N,N-dimethylethan-1-amine (P-101)

To an ice-cold (0° C.) solution of 2-(7-fluoro-4-methoxy-1H-indol-3-yl)ethan-1-amine (300 mg, 1.44 mmol) in MeOH (6 mL) was added AcOH (346 mg, 5.76 mmol), NaBH₃CN (181 mg, 2.88 mmol), and 37% w/w aqueous formaldehyde solution (292 mg, 3.60 mmol) and stirred at 20° C. for 3 h. The pH of the reaction mixture was then adjusted to 8 with saturated aqeuous Na₂CO₃ solution and the organic solvent was removed in vacuo. The residue was diluted with H₂O (20 mL) and extracted with EtOAc (10 mL×2). The combined organic layers were washed with brine (10 mL), dried over anhydrous Na₂SO₄, filtered, and the filtrate concentrated in vacuo. The crude product was purified by preparative HPLC (column: C18-4 150×30 mm×5 μm; mobile phase: [water (TFA)-ACN]; B: 10-40%, 20 min) to afford 2-(7-fluoro-4-methoxy-1H-indol-3-yl)-N,N-dimethylethan-1-amine (58.7 mg, 17%) as a white solid. ¹H NMR (400 MHZ, MeOD-d₄): δ 6.96 (s, 1H), 6.68 (dd, J=10.8, 8.4 Hz, 1H), 6.31 (dd, J=8.4, 2.8 Hz, 1H), 3.87 (s, 3H), 3.07-3.00 (m, 2H), 2.76-2.71 (m, 2H), 2.41 (s, 6H). ¹⁹F NMR (376 MHZ, MeOD-d₄): δ −146.9. LCMS (ESI+): m/z 237.2 [M+H]⁺. HPLC purity (220 nm): 99.9%.

### Example 75: N-ethyl-2-(7-fluoro-4-methoxy-1H-indol-3-yl)-N-methylethan-1-amine (P-102)

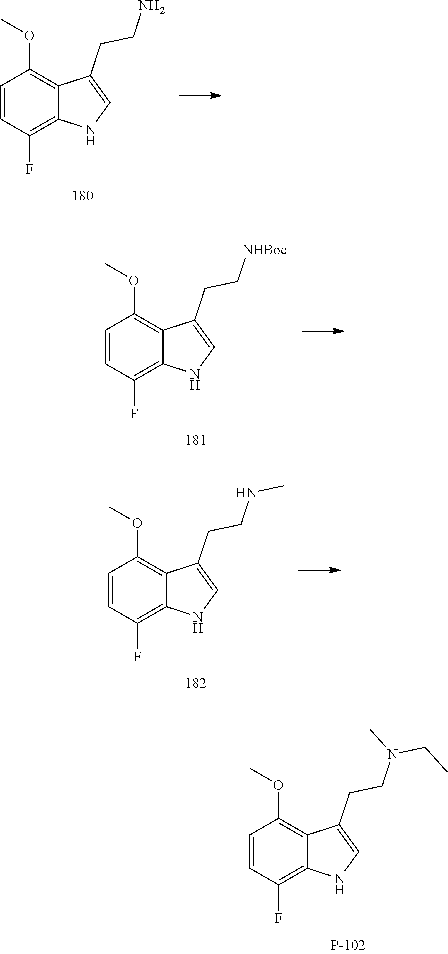 [SMILES: COc1ccc(F)c2[nH]cc(CCN)c12 ; COc1ccc(F)c2[nH]cc(CCNC(=O)OC(C)(C)C)c12 ; CNCCc1c[nH]c2c(F)ccc(OC)c12 ; CCN(C)CCc1c[nH]c2c(F)ccc(OC)c12]

### Step 1: tert-butyl (2-(7-fluoro-4-methoxy-1H-indol-3-yl)ethyl)carbamate (181)

To a solution of 2-(7-fluoro-4-methoxy-1H-indol-3-yl)ethan-1-amine (1.00 g, 4.80 mmol) in THF (7 mL) was added Boc₂O (1.10 g, 5.04 mmol), and the mixture was stirred at 25° C. for 2 h. The mixture was filtered and concentrated in vacuo to give crude tert-butyl (2-(7-fluoro-4-methoxy-1H-indol-3-yl)ethyl)carbamate (0.80 g) as a brown oil which was used in the subsequent step without further purification.

### Step 2: 2-(7-fluoro-4-methoxy-1H-indol-3-yl)-N-methylethan-1-amine (182)

To an ice-cold (0° C.) solution of crude tert-butyl (2-(7-fluoro-4-methoxy-1H-indol-3-yl)ethyl)carbamate (0.80 g) in anhydrous THF (5.60 mL) was added LiAlH₄ (492 mg, 12.9 mmol), and the mixture was stirred at 70° C. for 3 h. The mixture was cooled to 0° C., diluted with THF (10 mL), quenched with Na₂SO₄·10H₂O (5.00 g), and stirred at 20° C. for 30 min. The mixture was filtered, the filtrate was concentrated in vacuo to give crude 2-(7-fluoro-4-methoxy-1H-indol-3-yl)-N-methylethan-1-amine (0.30 g) as a brown oil which was used in the subsequent step without further purification.

### Step 3: N-ethyl-2-(7-fluoro-4-methoxy-1H-indol-3-yl)-N-methylethan-1-amine (P-102)

To a solution of crude 2-(7-fluoro-4-methoxy-1H-indol-3-yl)-N-methylethan-1-amine (0.30 g) in MeOH (3 mL) at 0° C. was added AcOH (324 mg, 5.40 mmol), NaBH₃CN (169 mg, 2.70 mmol), 40% w/w aqueous acetaldehyde (297 mg, 6.75 mmol), and the mixture was stirred at 20° C. for 16 h. The pH of the mixture was adjusted to 8 with saturated aqueous Na₂CO₃, and the MeOH was removed in vacuo. The residue was dissolved in H₂O (10 mL), extracted with EtOAc (10 mL×3), and the combined organic layers were washed with brine (10 mL), dried over Na₂SO₄, filtered, and the filtrate concentrated in vacuo. The residue was purified by preparative HPLC (column: Waters Xbridge Prep OBD C18 150×40 mm×10 μm; mobile phase: [water (NH₄HCO₃)-ACN]; B: 10-40%, 15 min) to give N-ethyl-2-(7-fluoro-4-methoxy-1H-indol-3-yl)-N-methylethan-1-amine (20.4 mg, 3% over 3 steps) as a yellow solid. ¹H NMR (400 MHZ, MeOD-d₄): δ 6.95 (s, 1H), 6.68 (dd, J=10.8, 8.4 Hz, 1H), 6.31 (dd, J=8.4, 3.2 Hz, 1H), 3.87 (s, 3H), 3.09-2.90 (m, 2H), 2.75-2.63 (m, 2H), 2.57 (q, J=7.2 Hz, 2H), 2.36 (s, 3H), 1.13 (t, J=7.2 Hz, 3H). ¹⁹F NMR (376 MHZ, MeOD-d₄): δ −147.0. LCMS (ESI+): m/z 251.2 [M+H]⁺; HPLC purity (220 nm): 97.8%.

### Example 76: N-(2-(7-fluoro-4-methoxy-1H-indol-3-yl)ethyl)-N-methylpropan-2-amine (P-103)

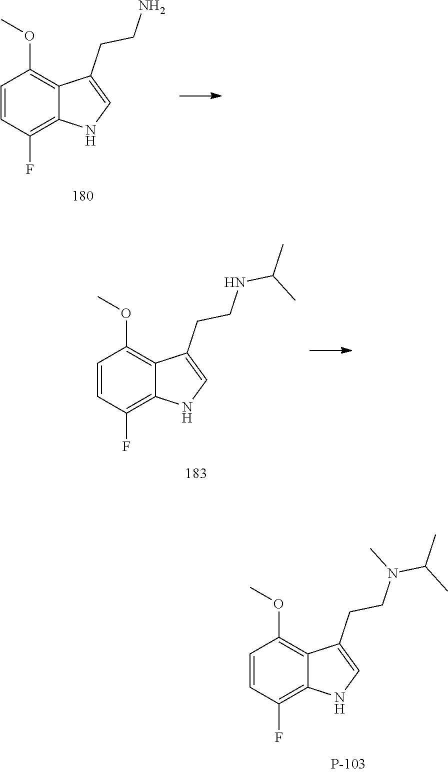 [SMILES: COc1ccc(F)c2[nH]cc(CCN)c12 ; COc1ccc(F)c2[nH]cc(CCNC(C)C)c12 ; COc1ccc(F)c2[nH]cc(CCN(C)C(C)C)c12]

### Step 1: N-(2-(7-fluoro-4-methoxy-1H-indol-3-yl)ethyl)propan-2-amine (183)

To an ice-cold (0° C.) solution of 2-(7-fluoro-4-methoxy-1H-indol-3-yl)ethan-1-amine (0.80 g, 3.84 mmol) in MeOH (8 mL) was added AcOH (922 mg, 15.3 mmol), NaBH₃CN (482 mg, 7.68 mmol), acetone (1.12 g, 19.2 mmol), and the mixture was stirred at 20° C. for 16 h. The pH of the mixture was adjusted to 8 with saturated aqueous Na₂CO₃, and the MeOH evaporated in vacuo. The residue was dissolved in

H₂O (20 mL), extracted with EtOAc (20 mL×3), and the combined organic layers were washed with brine (20 mL), dried over Na₂SO₄, filtered and the filtrate concentrated in vacuo. The residue was purified by preparative HPLC (column: Waters Xbridge Prep OBD C18 150×40 mm×10 μm; mobile phase: [water (NH₄HCO₃)-ACN]; B: 10-40%, 15 min) to give crude N-(2-(7-fluoro-4-methoxy-1H-indol-3-yl)ethyl)propan-2-amine (210 mg) as a yellow solid which was used in the subsequent step without further purification.

### Step 2: N-(2-(7-fluoro-4-methoxy-1H-indol-3-yl)ethyl)-N-methylpropan-2-amine (P-103)

To an ice-cold (0° C.) solution of crude N-(2-(7-fluoro-4-methoxy-1H-indol-3-yl)ethyl)propan-2-amine (210 mg) in MeOH (2.10 mL) was added AcOH (201 mg, 3.36 mmol), NaBH₃CN (125 mg, 4.19 mmol), 37% w/w aqueous formaldehyde solution (105 mg, 1.68 mmol), and the mixture was stirred at 20° C. for 16 h. The pH of the mixture was adjusted to 8 with saturated aqueous Na₂CO₃ and the MeOH was evaporated in vacuo. The residue was dissolved in H₂O (10 mL), extracted with EtOAc (10 mL×3), and the combined organic phases were washed with brine (10 mL), dried over Na₂SO₄, filtered, and the filtrate concentrated in vacuo. The residue was purified by preparative HPLC (column: Waters Xbridge Prep OBD C18 150×40 mm×10 μm; mobile phase: [water (NH₄HCO₃)-ACN]; B: 15-45%, 15 min) to give N-(2-(7-fluoro-4-methoxy-1H-indol-3-yl)ethyl)-N-methylpropan-2-amine (36.0 mg, 4% over 2 steps) as a yellow solid. ¹H NMR (400 MHZ, MeOD-d₄): δ 6.96 (s, 1H), 6.69 (dd, J=10.8, 8.4 Hz, 1H), 6.32 (dd, J=8.4, 3.0 Hz, 1H), 3.88 (s, 3H), 3.06-2.86 (m, 3H), 2.81-2.68 (m, 2H), 2.38 (s, 3H), 1.10 (d, J=6.8 Hz, 6H). ¹⁹F NMR (376 MHZ, MeOD-d₄): δ −147.0. LCMS (ESI+): m/z 265.1 [M+H]⁺. HPLC Purity (254 nm): 96.9%.

### Example 77: N,N-diethyl-2-(7-fluoro-4-methoxy-1H-indol-3-yl)ethan-1-amine (P-104)

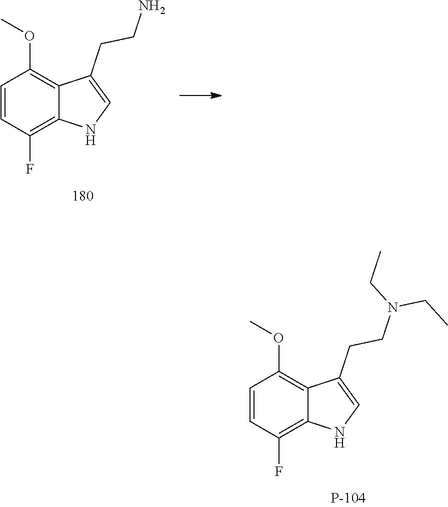 [SMILES: COc1ccc(F)c2[nH]cc(CCN)c12 ; CCN(CC)CCc1c[nH]c2c(F)ccc(OC)c12]

### Step 1: N,N-diethyl-2-(7-fluoro-4-methoxy-1H-indol-3-yl)ethan-1-amine (P-104)

To an ice-cold (0° C.) solution of 2-(7-fluoro-4-methoxy-1H-indol-3-yl)ethan-1-amine (900 mg, 4.32 mmol) in MeOH (9 mL) was added AcOH (1.04 g, 17.2 mmol), NaBH₃CN (543 mg, 8.64 mmol), CH₃CHO (476 mg, 10.8 mmol), and the mixture was stirred at 20° C. for 16 h. The pH of the mixture was adjusted to 8 with saturated aqueous Na₂CO₃, and the MeOH evaporated in vacuo. The residue was dissolved in H₂O (20 mL), extracted with EtOAc (20 mL×3), and the combined organic layers were washed with brine (20 mL), dried over Na₂SO₄, filtered, and the filtrate concentrated in vacuo. The residue was purified by preparative HPLC (column: Waters Xbridge Prep OBD C18 150×40 mm×10 μm; mobile phase: [water (NH₄HCO₃)-ACN]; B: 15-45%, 15 min) to give N, N-diethyl-2-(7-fluoro-4-methoxy-1H-indol-3-yl)ethan-1-amine (39.0 mg, 3%) as a yellow solid. ¹H NMR (400 MHZ, MeOD-d₄): δ 6.95 (s, 1H), 6.68 (dd, J=10.8, 8.4 Hz, 1H), 6.31 (dd, J=8.4, 3.2 Hz, 1H), 3.87 (s, 3H), 3.02-2.96 (m, 2H), 2.80-2.74 (m, 2H), 2.68 (q, J=7.2 Hz, 4H), 1.13 (t, J=7.2 Hz, 6H); ¹⁹F NMR (376 MHZ, MeOD-d₄): δ −147.0. LCMS (ESI+): m/z 265.1 [M+H]⁺; HPLC Purity (254 nm): 96.3%.

### Example 78: N-(2-(7-fluoro-4-methoxy-1H-indol-3-yl)ethyl)-N-isopropylpropan-2-amine (P-105)

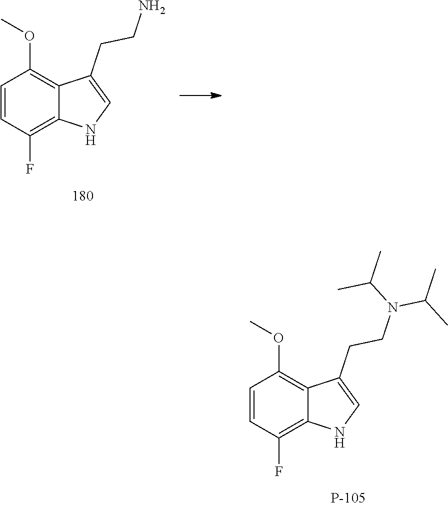 [SMILES: COc1ccc(F)c2[nH]cc(CCN)c12 ; COc1ccc(F)c2[nH]cc(CCN(C(C)C)C(C)C)c12]

### Step 1: N-(2-(7-fluoro-4-methoxy-1H-indol-3-yl)ethyl)-N-isopropylpropan-2-amine (P-105)

To a solution of 2-(7-fluoro-4-methoxy-1H-indol-3-yl)ethan-1-amine (800 mg, 3.84 mmol) in MeOH (8 mL) at 0° C. was added AcOH (922 mg, 15.3 mmol), NaBH₃CN (4.46 g, 76.8 mmol), acetone (4.46 g, 76.8 mmol), and the mixture was stirred at 20° C. for 16 h. The pH of the reaction mixture was adjusted to 8 with saturated aqueous Na₂CO₃, and the MeOH was evaporated in vacuo. The residue was dissolved in H₂O (20 mL), extracted with EtOAc (20 mL×3), and the combined organic layers were washed with brine (20 mL), dried over Na₂SO₄, filtered, and concentrated in vacuo. The residue was purified by preparative HPLC (column: Waters Xbridge 150×25 mm×5 μm; mobile phase: [water (NH₄HCO₃)-ACN]; B: 20-50%, 9 min) to give N-(2-(7-fluoro-4-methoxy-1H-indol-3-yl)ethyl)-N-isopropylpropan-2-amine (23.0 mg, 2%) as a white solid. ¹H NMR (400 MHZ, MeOD-d₄): δ 6.94 (s, 1H), 6.68 (dd, J=10.8, 8.4 Hz, 1H), 6.31 (dd, J=8.4, 3.2 Hz, 1H), 3.87 (s, 3H), 3.13 (sept, J=6.8 Hz, 2H), 3.03-2.86 (m, 2H), 2.81-2.63 (m, 2H), 1.14 (d, J=6.8 Hz, 12H). ¹⁹F NMR (376 MHZ, MeOD-d₄): δ −147.0; LCMS (ESI+): m/z 293.1 [M+H]⁺; HPLC purity (220 nm): 99.0%.

Scheme 18: Compounds of general formula (I) can be synthesised from the appropriately substituted indole following the outlined sequence of steps in Scheme 18 or similar as one skilled in the art may consider. Glyoxylation of substituted indoles with oxalyl chloride followed by treatment with an appropriately substituted amine gave glyoxamide intermediates which, when subjected to reductive conditions, provided compounds of general formula (I) (exemplified by P-106). One skilled in the art will recognise that utilising differentially substituted amines would allow access to compounds of general formula (I) disclosed herein.

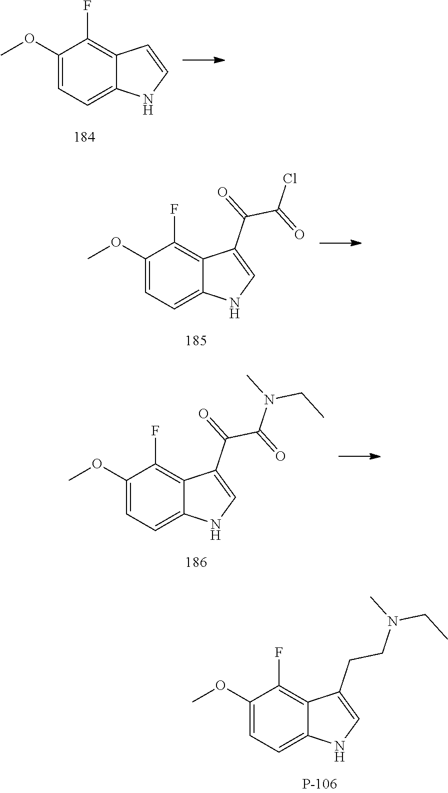

### Example 79: N-ethyl-2-(4-fluoro-5-methoxy-1H-indol-3-yl)-N-methylethan-1-amine (P-106)

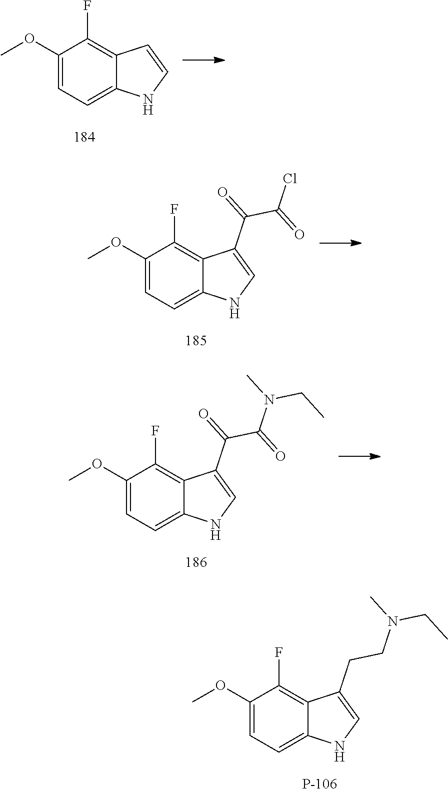 [SMILES: COc1ccc2[nH]ccc2c1F ; COc1ccc2[nH]cc(C(=O)C(=O)Cl)c2c1F ; CCN(C)C(=O)C(=O)c1c[nH]c2ccc(OC)c(F)c12 ; CCN(C)CCc1c[nH]c2ccc(OC)c(F)c12]

### Step 1: 2-(4-fluoro-5-methoxy-1H-indol-3-yl)-2-oxoacetyl chloride (185)

Oxalyl chloride (1.35 mL, 15.7 mmol) was added dropwise to an ice-cold stirred solution of 4-fluoro-5-methoxy-1H-indole (2 g, 12.1 mmol) in anhydrous Et₂O (25 mL) over 20 min. Stirring was continued at this temperature for 2 h. The resulting precipitate was then collected by suction filtration and washed with Et₂O (2×20 mL). The solid (2.7 g, 87%) was collected and dried under vacuum for 2 h before being subjected to the next step without further purification.

### Step 2: N-ethyl-2-(4-fluoro-5-methoxy-1H-indol-3-yl)-N-methyl-2-oxoacetamide (186)

A solution of ethyl(methyl)amine (143 mg, 2.2 eq., 2.42 mmol) in CH₂Cl₂ (5 mL) was added dropwise to an ice-cold suspension of 2-(4-fluoro-5-methoxy-1H-indol-3-yl)-2-oxoacetyl chloride (281 mg, 1.1 mmol). Stirring was continued for 3 h at which point the reaction was diluted with H₂O (20 mL) and organic layer separated. The organic layer was washed subsequently with dilute HCl (0.1 M aq., 25 mL), H₂O (2×25 mL) followed by brine (50 mL) before being dried (MgSO₄), filtered, and concentrated under reduced pressure. The residue was triturated with CH₂Cl₂/Et₂O and filtered to give a white powder identified as the desired product N-ethyl-2-(4-fluoro-5-methoxy-1H-indol-3-yl)-N-methyl-2-oxoacetamide (224 mg, 0.81 mmol, 73%). ¹H NMR (400 MHz, MeOD-d₄): δ 8.08-8.00 (m, 1H), 7.25 (dd, J=8.8, 0.9 Hz, 1H), 7.15 (dd, J=8.8, 7.4 Hz, 1H), 3.92 (s, 3H), 3.58 (q, J=7.2 Hz, 1H), 3.40 (q, J=7.1 Hz, 1H), 3.10-2.99 (m, 3H), 1.34-1.16 (m, 3H).

### Step 3: N-ethyl-2-(4-fluoro-5-methoxy-1H-indol-3-yl)-N-methylethan-1-amine (P-106)

To an ice-cold stirred solution of N-ethyl-2-(4-fluoro-5-methoxy-1H-indol-3-yl)-N-methyl-2-oxoacetamide (330 mg, 1.19 mmol) in anhydrous THF (15 mL) was added LiAlH₄ (135 mg, 3 eq., 3.56 mmol) in portions. The resulting suspension was then heated at reflux for 1 h. The suspension was cooled in an ice bath and quenched by subsequent addition of H₂O (150 μL), 30% NaOH (w/v) (150 μL), H₂O (450 μL). The suspension was stirred at 0° C. for a further 1 h, Na₂SO₄ was added, and the reaction mass filtered through a pad of celite, and washed with THF (2×20 mL). The filtrates were concentrated under a stream of N₂ gas to give N-ethyl-2-(4-fluoro-5-methoxy-1H-indol-3-yl)-N-methylethan-1-amine (240 mg) as a colourless oil which was used in the subsequent step without further purification.

### Step 3a: N-ethyl-2-(4-fluoro-5-methoxy-1H-indol-3-yl)-N-methylethan-1-amine oxalate (P-106·oxalate)

A solution of N-ethyl-2-(4-fluoro-5-methoxy-1H-indol-3-yl)-N-methylethan-1-amine (244 mg, 0.90 mmol) in hot acetone (5 mL) was added dropwise to a saturated solution of oxalic acid (81 mg, 0.90 mmol) in acetone at reflux. The reaction solution was allowed to cool to room temperature and the resultant yellow/orange crystals were collected by vacuum filtration and dried in a vacuum desiccator overnight yielding N-ethyl-2-(4-fluoro-5-methoxy-1H-indol-3-yl)-N-methylethan-1-amine (218 mg, 73% over 2 steps) as the oxalate salt. ¹H NMR (400 MHZ, DMSO-d₆): δ 11.10 (s, 1H), 7.24 (d, J=2.2 Hz, 1H), 7.11 (d, J=8.8 Hz, 1H), 6.98 (t, J=8.4 Hz, 1H), 3.82 (s, 3H), 3.29-3.03 (m, 6H), 2.77 (s, 3H), 1.22 (t, J=7.2 Hz, 3H). ¹³C NMR (101 MHz, DMSO): δ 165.0, 147.0, 144.6, 139.5 (d, J=9.4 Hz), 134.5 (d, J=10.4 Hz), 125.8, 116.8, 111.9, 107.7 (d, J=16.7 Hz), 58.6, 55.8, 50.5, 21.4, 9.5. qNMR Purity (ERETIC): 96.1%.

### Example 80: N-(2-(4-fluoro-5-methoxy-1H-indol-3-yl)ethyl)-N-methylpropan-2-amine (P-107)

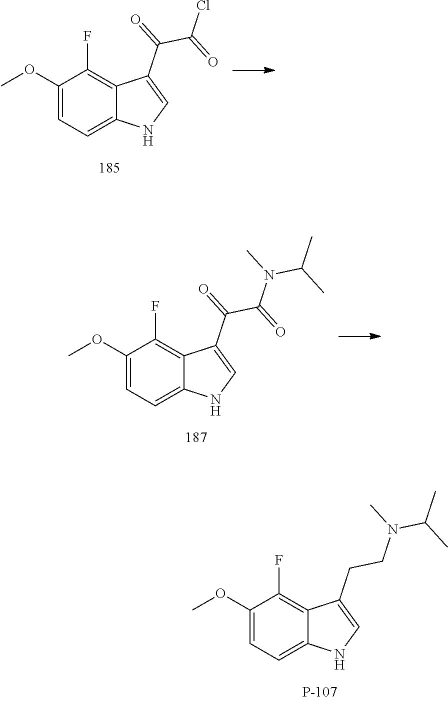 [SMILES: COc1ccc2[nH]cc(C(=O)C(=O)Cl)c2c1F ; COc1ccc2[nH]cc(C(=O)C(=O)N(C)C(C)C)c2c1F ; COc1ccc2[nH]cc(CCN(C)C(C)C)c2c1F]

### Step 1: 2-(4-fluoro-5-methoxy-1H-indol-3-yl)-N-isopropyl-N-methyl-2-oxoacetamide (187)

A solution of methyl(propan-2-yl)amine (189 mg, 2.58 mmol) in CH₂Cl₂ (5 mL) was added dropwise to an ice-cold suspension of 2-(4-fluoro-5-methoxy-1H-indol-3-yl)-2-oxoacetyl chloride (0.3 g, 1.17 mmol). Stirring was continued for 3 h at which point the reaction was diluted with H₂O (20 mL) and organic layer separated. The organic layer was washed subsequently with dilute HCl (0.1 M aq., 25 mL), H₂O (2×25 mL) followed by brine (50 mL) before being dried (MgSO₄), filtered, and concentrated under reduced pressure. The residue was triturated with CH₂Cl₂/Ether and filtered to give a white powder identified as the desired product 2-(4-fluoro-5-methoxy-1H-indol-3-yl)-N-isopropyl-N-methyl-2-oxoacetamide (251 mg, 73%). ¹H NMR (400 MHZ, MeOD-d₄): δ 8.21-8.02 (m, 1H), 7.59-6.96 (m, 3H), 4.10-3.93 (m, 1H), 3.09-2.85 (m, 3H), 1.31-1.21 (m, 6H) [mixture of 2 rotamers].

### Step 2: N-(2-(4-fluoro-5-methoxy-1H-indol-3-yl)ethyl)-N-methylpropan-2-amine (P-107)

To an ice-cold stirred solution of 2-(4-fluoro-5-methoxy-1H-indol-3-yl)-N-isopropyl-N-methyl-2-oxoacetamide (341 mg, 1.17 mmol) in anhydrous THF (15 mL) was added LiAlH₄ (133 mg, 3 eq., 3.5 mmol) in portions. The resulting suspension was then heated at reflux for 1 h. The suspension was cooled in an ice bath and quenched by subsequent addition of H₂O (200 μL), 30% NaOH (w/v) (200 μL), H₂O (600 μL). The suspension was stirred at 0° C. for a further 1 h, Na₂SO₄ was added, and the reaction mass filtered through a pad of celite and the residue washed with THF (2×20 mL). The filtrate was concentrated under a stream of N₂ gas to give N-(2-(4-fluoro-5-methoxy-1H-indol-3-yl)ethyl)-N-methylpropan-2-amine (288 mg) as a colourless oil which was used in the subsequent step without further purification.

### Step 2a: N-(2-(4-fluoro-5-methoxy-1H-indol-3-yl)ethyl)-N-methylpropan-2-amine fumarate (P-107⋅fumarate)

A solution of N-(2-(4-fluoro-5-methoxy-1H-indol-3-yl)ethyl)-N-methylpropan-2-amine (271 mg, 1.03 mmol) in hot acetone (2 mL) was added dropwise to a saturated solution of fumaric acid (119 mg, 1.03 mmol) in acetone at reflux. The colorless solution was allowed to cool to ambient temperature and stood at this temperature for 16 h. The resultant crystals were collected by vacuum filtration and dried in a vacuum desiccator to afford N-(2-(4-fluoro-5-methoxy-1H-indol-3-yl)ethyl)-N-methylpropan-2-amine as the fumarate salt (247 mg, 56% over 2 steps). ¹H NMR (400 MHZ, DMSO-d₆): δ 10.99 (s, 1H), 7.18 (d, J=2.4 Hz, 1H), 7.08 (d, J=8.8 Hz, 1H), 7.02-6.90 (m, 1H), 6.52 (s, 2H), 3.81 (s, 3H), 3.34-3.17 (m, 1H), 3.08-2.85 (m, 4H), 2.50-2.48 (m, 3H), 1.11 (d, J=6.8 Hz, 6H). ¹³C NMR (101 MHZ, DMSO-d₆): δ 167.3, 145.5 (d, J=242.1 Hz), 138.9 (d, J=9.5 Hz), 134.8, 134.0 (d, J=10.7 Hz), 124.9, 116.4 (d, J=17.2 Hz), 111.2, 108.8, 107.1 (d, J=4.0 Hz), 58.1, 54.3, 53.7, 35.6, 22.8, 16.7. qNMR Purity (ERETIC): 95.4%.

### Example 81: N,N-diethyl-2-(4-fluoro-5-methoxy-1H-indol-3-yl)ethan-1-amine (P-108)

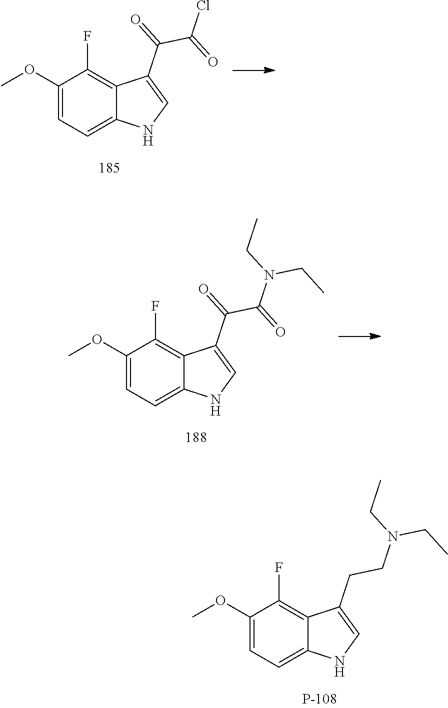 [SMILES: COc1ccc2[nH]cc(C(=O)C(=O)Cl)c2c1F ; CCN(CC)C(=O)C(=O)c1c[nH]c2ccc(OC)c(F)c12 ; CCN(CC)CCc1c[nH]c2ccc(OC)c(F)c12]

### Step 1: N,N-diethyl-2-(4-fluoro-5-methoxy-1H-indol-3-yl)-2-oxoacetamide (188)

A solution of diethylamine (159 mg, 2.18 mmol) in CH₂Cl₂ (5 mL) was added dropwise to an ice-cold suspension of 2-(4-fluoro-5-methoxy-1H-indol-3-yl)-2-oxoacetyl chloride (253 mg, 0.99 mmol). Stirring was continued for 3 h at which point the reaction was diluted with H₂O (20 mL) and organic layer separated. The organic layer was washed subsequently with dilute HCl (0.1 M aq., 25 mL), H₂O (2×25 mL) followed by brine (50 mL) before being dried (MgSO₄), filtered, and concentrated under reduced pressure. The residue was triturated with CH₂Cl₂/Ether and filtered to give a white powder identified as the desired product, N,N-diethyl-2-(4-fluoro-5-methoxy-1H-indol-3-yl)-2-oxoacetamide (257 mg, 89%). ¹H NMR (400 MHZ, DMSO): δ 12.50 (s, 1H), 8.32 (d, J=3.4 Hz, 1H), 7.29 (dd, J=8.8, 0.4 Hz, 1H), 7.18 (dd, J=8.8, 7.6 Hz, 1H), 3.85 (s, 3H). ¹³C NMR (101 MHZ, DMSO): δ 180.10, 166.28 (d, J=3.3 Hz), 145.03 (d, J=251.3 Hz), 142.54 (d, J=10.3 Hz), 139.05, 133.90 (d, J=9.5 Hz), 114.17 (d, J=18.4 Hz), 112.64, 111.50 (d, J=5.5 Hz), 108.30 (d, J=4.4 Hz), 57.57.

### Step 2: N,N-diethyl-2-(4-fluoro-5-methoxy-1H-indol-3-yl)ethan-1-amine (P-108)

To an ice-cold stirred solution of N, N-diethyl-2-(4-fluoro-5-methoxy-1H-indol-3-yl)-2-oxoacetamide (325 mg, 1.11 mmol) in anhydrous THF (15 mL) was added LiAlH₄ (127 mg, 3 eq., 3.3 mmol) in portions. The resulting suspension was then heated at reflux for 1 h. The suspension was cooled in an ice bath and quenched by subsequent addition of H₂O (100 μL), 30% NaOH (w/v) (100 μL), H₂O (300 μL). The suspension was stirred at 0° C. for a further 1 h, Na₂SO₄ was added, and the reaction mass filtered through a pad of celite and the residue washed with THF (2×20 mL). The filtrate was concentrated under a stream of N₂ gas to give N,N-diethyl-2-(4-fluoro-5-methoxy-1H-indol-3-yl)ethan-1-amine (250 mg) as a colourless oil which was used in the subsequent step without further purification.

### Step 2a: N,N-diethyl-2-(4-fluoro-5-methoxy-1H-indol-3-yl)ethan-1-amine fumarate (P-108⋅fumarate)

A solution of N, N-diethyl-2-(4-fluoro-5-methoxy-1H-indol-3-yl)ethan-1-amine (237 mg, 0.90 mmol) in hot acetone (2 mL) was added dropwise to a saturated solution of fumaric acid (104 mg, 0.90 mmol) in acetone at reflux. The colorless solution was allowed to cool to ambient temperature and stood at this temperature for 16 h. The resultant crystals were collected by vacuum filtration and dried in a vacuum desiccator to afford N,N-diethyl-2-(4-fluoro-5-methoxy-1H-indol-3-yl)ethan-1-amine as the fumarate salt (218 mg, 51% over 2 steps). ¹H NMR (400 MHZ, DMSO-d₆): δ 11.02 (s, 1H), 7.21 (d, J=2.4 Hz, 1H), 7.09 (d, J=8.8 Hz, 1H), 7.00-6.92 (m, 1H), 6.54 (s, 3H), 3.81 (s, 3H), 3.10-2.87 (m, 8H), 1.15 (t, J=7.2 Hz, 6H). qNMR Purity (ERETIC): 98.8%.

### Example 82: N-(2-(4-fluoro-5-methoxy-1H-indol-3-yl)ethyl)-N-isopropylpropan-2-amine (P-109)

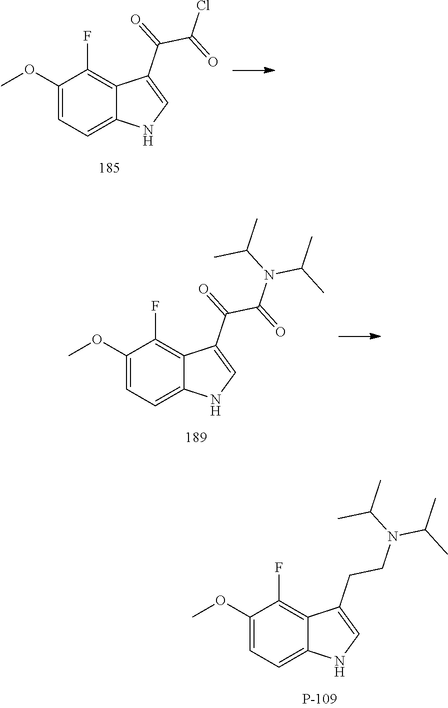 [SMILES: COc1ccc2[nH]cc(C(=O)C(=O)Cl)c2c1F ; COc1ccc2[nH]cc(C(=O)C(=O)N(C(C)C)C(C)C)c2c1F ; COc1ccc2[nH]cc(CCN(C(C)C)C(C)C)c2c1F]

### Step 1: 2-(4-fluoro-5-methoxy-1H-indol-3-yl)-N,N-diisopropyl-2-oxoacetamide (189)

A solution of diisopropylamine (804 mg, 7.95 mmol) in CH₂Cl₂ (5 mL) was added dropwise to an ice-cold suspension of 2-(4-fluoro-5-methoxy-1H-indol-3-yl)-2-oxoacetyl chloride (406 mg, 1.59 mmol). Stirring was continued for 3 h at which point the reaction was diluted with H₂O (20 mL) and organic layer separated. The organic layer was washed subsequently with dilute HCl (0.1 M aq., 25 mL), H₂O (2×25 mL) followed by brine (50 mL) before being dried (MgSO₄), filtered, and the filtrate concentrated under reduced pressure. The residue was triturated with CH₂Cl₂/Et₂O and filtered to give a white powder identified as the desired product, 2-(4-fluoro-5-methoxy-1H-indol-3-yl)-N,N-diisopropyl-2-oxoacetamide (286 mg, 56%). ¹H NMR (400 MHZ, CDCl₃): δ 10.72 (s, 1H), 7.56 (d, J=2.4 Hz, 1H), 7.47 (d, J=3.3 Hz, 1H), 6.64 (dd, J=2.4, 1.1 Hz, 1H), 3.93 (sept, J=6.6 Hz, 1H), 3.85 (s, 3H), 3.54 (sept, J=6.8 Hz, 1H), 2.34 (s, 3H), 1.54 (d, J=6.8 Hz, 6H), 1.13 (d, J=6.6 Hz, 6H).

### Step 2: N-(2-(4-fluoro-5-methoxy-1H-indol-3-yl)ethyl)-N-isopropylpropan-2-amine (P-109)

To an ice-cold stirred solution of 2-(4-fluoro-5-methoxy-1H-indol-3-yl)-N,N-diisopropyl-2-oxoacetamide (390 mg, 1.22 mmol) in anhydrous THF (15 mL) was added LiAlH₄ (139 mg, 3 eq., 3.65 mmol) in portions. The resulting suspension was then heated at reflux for 1 h. The suspension was cooled in an ice bath and quenched by subsequent addition of H₂O (150 μL), 30% NaOH (w/v) (150 μL), H₂O (750 μL). The suspension was stirred at 0° C. for a further 1 h, Na₂SO₄ was added, and the reaction mass filtered through a pad of celite, and the residue washed with THF (2×20 mL). The filtrate was concentrated under a stream of N₂ gas to give N-(2-(4-fluoro-5-methoxy-1H-indol-3-yl)ethyl)-N-isopropylpropan-2-amine (301 mg) as a colourless oil which was used in the subsequent step without further purification.

### Step 2a: N-(2-(4-fluoro-5-methoxy-1H-indol-3-yl)ethyl)-N-isopropylpropan-2-amine fumarate (P-109-fumarate)

A solution of N-(2-(4-fluoro-5-methoxy-1H-indol-3-yl)ethyl)-N-isopropylpropan-2-amine (286 mg, 0.98 mmol) in hot acetone (2 mL) was added dropwise to a saturated solution of fumaric acid (114 mg, 0.98 mmol) in acetone at reflux. The colorless solution was allowed to cool to ambient temperature and stood at this temperature for 16 h. The resultant crystals were collected by vacuum filtration and dried in a vacuum desiccator to afford N-(2-(4-fluoro-5-methoxy-1H-indol-3-yl)ethyl)-N-isopropylpropan-2-amine as the fumarate salt (281 mg, 79% over 2 steps). ¹H NMR (400 MHZ, DMSO-d₆): δ 11.03 (s, 1H), 7.22 (d, J=2.0 Hz, 1H), 7.09 (d, J=8.8 Hz, 1H), 7.01-6.89 (m, 1H), 6.54 (s, 2H), 3.81 (s, 3H), 3.45-3.30 (m, 2H), 3.03-2.85 (m, 4H), 1.17 (d, J=6.4 Hz, 12H). ¹³C NMR (101 MHZ, DMSO-d₆): δ 167.2, 145.4 (d, J=242.2 Hz), 138.8 (d, J=9.5 Hz), 134.8, 134.0 (d, J=10.7 Hz), 125.0, 116.4 (d, J=16.8 Hz), 111.1, 108.9, 107.1, 58.1, 51.2, 47.6, 26.3, 18.9. qNMR Purity (ERETIC): 95.6%.

Scheme 19: Compounds of general formula (I) can be synthesised from the appropriately substituted indole following the outlined sequence of steps in Scheme 19 or similar as one skilled in the art may consider. Glyoxylation of substituted indoles with oxalyl chloride followed by treatment with an appropriately substituted amine gave glyoxamide intermediates which, when subjected to reductive conditions, provided compounds of general formula (I) (exemplified by P-110). One skilled in the art will recognise that utilising differentially substituted amines would allow access to compounds of general formula (I) disclosed herein.

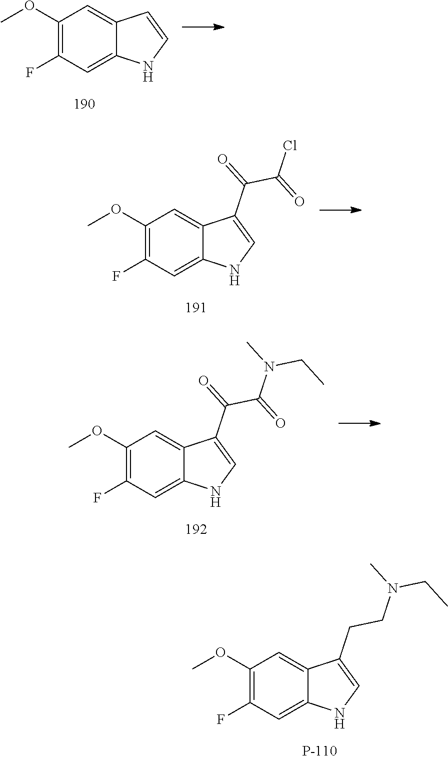

Scheme 20: Compounds of general formula (I) can be synthesised from the appropriately substituted benzaldehyde following a sequence of synthetic steps outlined in Scheme 20 or similar as one skilled in the art may consider. Initial Knovenagel condensation of substituted benzaldehydes such as 196 with ethyl azidoacetate gives rise to vinyl azide 197 which can undergo a thermally induced intramolecular cyclisation to furnish indole 198. Standard ester hydrolysis and subsequent copper mediated thermal decarboxylation gave indole 200. Such indole cores could then be condensed with dimethylamino-2-nitroethylene to give nitrovinyl indoles. Reduction of such indoles proved viable in accessing unsubstituted ethyl amine analogues that could subsequently undergo reductive alkylation to access compounds of general formula (I) exemplified by P-117. One skilled in the art will recognise that intermediate 202 can be condensed with boc-anhydride then subjected to reductive conditions generating mono-methyl intermediate 204 that can undergo subsequent reductive alkylations with various aldehydes and ketones providing access to differentially alkylated compounds of general formula (I, exemplified by P-115) disclosed herein.

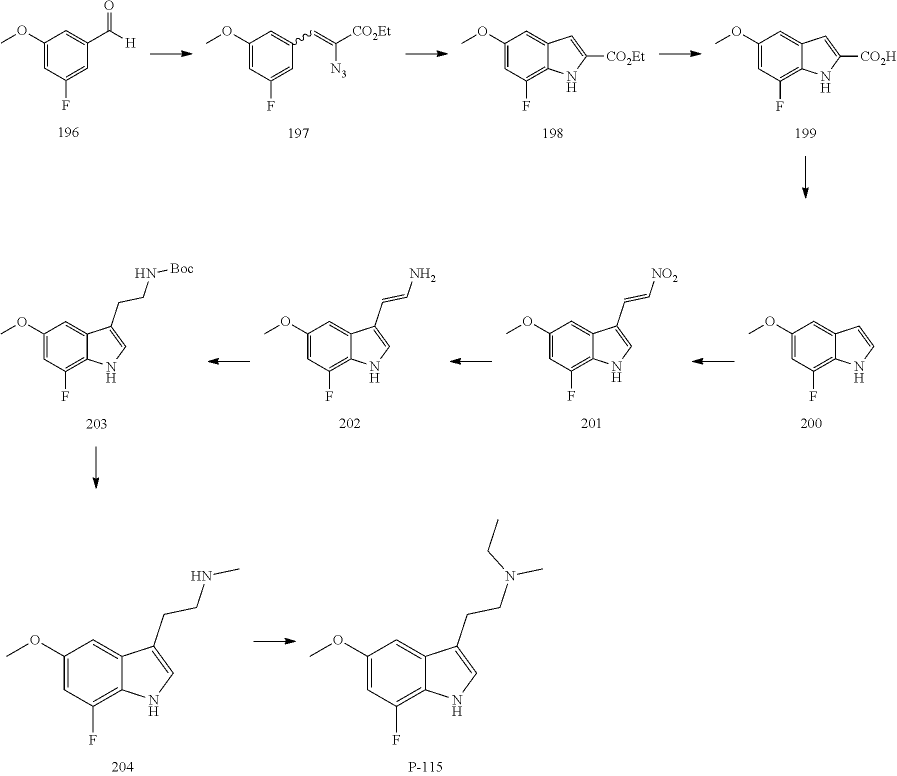

### Example 87: 2-(7-fluoro-4-methoxy-1H-indol-3-yl)-N,N-dimethylethan-1-amine (P-115)

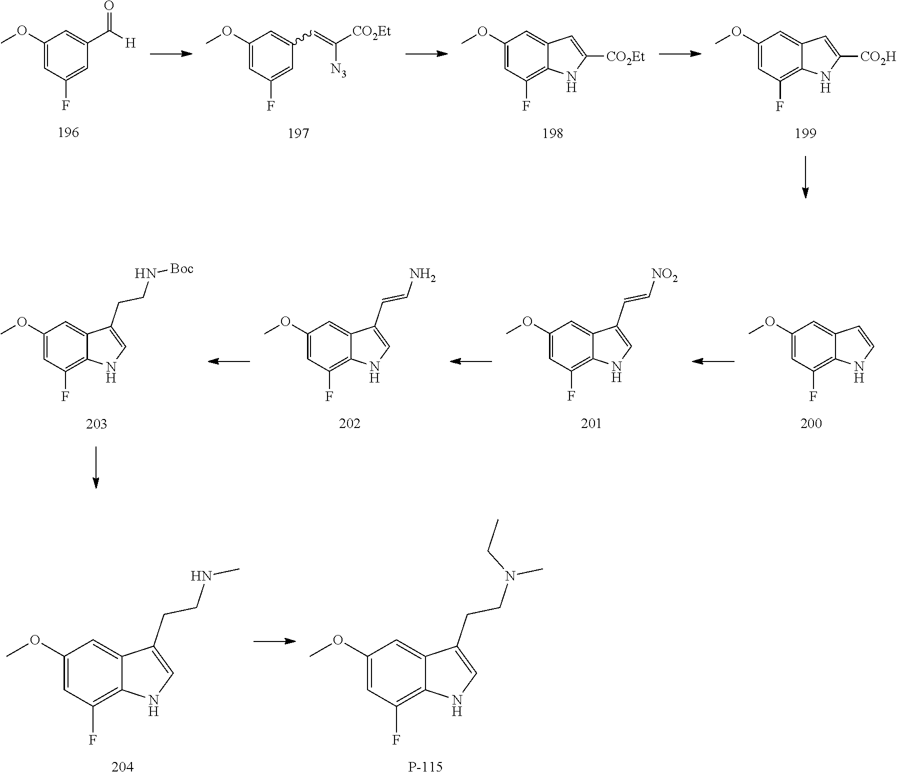

### Step 1: ethyl 2-azido-3-(3-fluoro-5-methoxyphenyl)acrylate (197)

To a mixture of 3-fluoro-5-methoxybenzaldehyde (200 g, 1.30 mol) and ethyl 2-azidoacetate (503 g, 3.89 mol, 446 mL) in EtOH (1.00 L) was added EtONa (265 g, 3.89 mol) at −20° C., the mixture was allowed to warm to 0° C. and stirring continued for 12 h. The reaction mixture was quenched by addition of saturated aqueous NH₄Cl solution (1.00 L) at 10° C., and then extracted with EtOAc (400 mL×3). The combined organic layers were washed with brine (300 mL×2), dried over Na₂SO₄, filtered, and the filtrate concentrated under reduced pressure. The residue was purified by column chromatography (SiO₂, petroleum ether/EtOAc, v/v, 100/1) to give ethyl 2-azido-3-(3-fluoro-5-methoxyphenyl)acrylate (152 g, 44% yield) as a white solid. ¹H NMR (400 MHz, CDCl₃): δ 7.21 (d, J=9.6 Hz, 1H), 7.12 (s, 1H), 6.80 (s, 1H), 6.61 (d, J=10.4 Hz, 1H), 4.38 (q, J=7.2 Hz, 2H), 3.83 (s, 3H), 1.41 (t, J=7.2 Hz, 3H).

### Step 2: ethyl 7-fluoro-5-methoxy-1H-indole-2-carboxylate (198)

A mixture of ethyl 2-azido-3-(3-fluoro-5-methoxyphenyl)acrylate (152 g, 573 mmol) in xylenes (912 mL) was stirred at 130° C. for 1 h, and then the cooled mixture was concentrated in vacuo. The residue was purified by column chromatography (SiO₂, petroleum ether/EtOAc, v/v, 50/1 to 20/1) to give crude ethyl 7-fluoro-5-methoxy-1H-indole-2-carboxylate (45.0 g) as a white solid which was used in the subsequent step without further purification.

### Step 3: 7-fluoro-5-methoxy-1H-indole-2-carboxylic acid (199)

Ethyl 7-fluoro-5-methoxy-1H-indole-2-carboxylate (45.0 g) was added to a solution of KOH (26.6 g, 474 mmol) in EtOH (180 mL) and H₂O (45 mL) and the mixture was stirred at 60° C. for 4 h. The volatile components were removed in vacuo and the aqueous residue was diluted with H₂O (400 mL). The pH was adjusted to 3 with 1 M aqueous HCl, and then extracted with EtOAc (500 mL×2). The combined organic layers were washed with brine (400 mL), dried over Na₂SO₄, filtered and the filtrate concentrated in vacuo to give crude 7-fluoro-5-methoxy-1H-indole-2-carboxylic acid (37.0 g) as a white solid which was used in the subsequent step without further purification.

### Step 4: 7-fluoro-5-methoxy-1H-indole (200)

A mixture of crude 7-fluoro-5-methoxy-1H-indole-2-carboxylic acid (18.5 g, 88.5 mmol) and Cu (3.93 g, 61.9 mmol) in quinoline (130 mL) was stirred at 230° C. for 2 h. The mixture was cooled to ambient temperature and diluted with EtOAc (600 mL). The resulting mixture was washed sequentially with 1 M aqueous HCl (300 ml×3), saturated aqueous NaHCO₃ solution (300 mL×2), brine (200 mL), dried over Na₂SO₄, filtered, and the filtrate concentrated in vacuo. The residue was purified by column chromatography (SiO₂, petroleum ether/EtOAc, v/v, 100/1 to 60/1) to give 7-fluoro-5-methoxy-1H-indole (6.00 g, 6% over 3 steps) as a yellow oil. ¹H NMR (400 MHZ, CDCl₃): δ 8.24 (br. s, 1H), 7.21-7.18 (m, 1H), 6.91 (d, J=2.0 Hz, 1H), 6.66 (dd, J=12.4, 2.0 Hz, 1H), 6.55-6.52 (m, 1H), 3.87 (s, 3H).

### Step 5: 7-fluoro-5-methoxy-3-(2-nitrovinyl)-1H-indole (201)

A mixture of 7-fluoro-5-methoxy-1H-indole (6.00 g, 36.3 mmol) and (E)-N,N-dimethyl-2-nitroethen-1-amine (4.22 g, 36.3 mmol) in TFA (36 mL) was stirred at 20° C. for 1 h. The mixture was adjusted to pH 8 with saturated aqueous NaHCO₃ solution, extracted with EtOAc (100 mL×2), and the combined organic layers were washed with brine (100 mL), dried over Na₂SO₄, filtered, and the filtrate concentrated in vacuo. The residue was purified by column chromatography (SiO₂, petroleum ether/EtOAc, v/v, 50/1 to 10/1) to give 7-fluoro-5-methoxy-3-(2-nitrovinyl)-1H-indole (3.50 g, 41% yield) as a red solid. ¹H NMR (400 MHZ, DMSO-d₆): δ 12.57 (br s, 1H), 8.41 (d, J=13.6 Hz, 1H), 8.25 (s, 1H), 8.07 (d, J=13.6 Hz, 1H), 7.26 (d, J=2.0 Hz, 1H), 6.81 (dd, J=12.4, 2.0 Hz, 1H), 3.86 (s, 3H).

### Step 6: 2-(7-fluoro-5-methoxy-1H-indol-3-yl)ethan-1-amine (202)

A solution of 7-fluoro-5-methoxy-3-(2-nitrovinyl)-1H-indole (3.50 g, 14.8 mmol) in THF (70 mL) at 0° C. was treated portionwise with LiAlH₄ (2.50 M in THF, 65.2 mL), and the mixture stirred at 80° C. for 2 h. The mixture was cooled to 0° C., diluted with THF (500 mL), and quenched with Na₂SO₄·10H₂O (30.0 g). The mixture was stirred at 20° C. for 30 min, filtered, and the filtrate was concentrated in vacuo to give crude 2-(7-fluoro-5-methoxy-1H-indol-3-yl)ethan-1-amine (4.00 g) as a brown oil.

### Step 7: tert-butyl (2-(7-fluoro-5-methoxy-1H-indol-3-yl)ethyl)carbamate (203)

A solution of crude 2-(7-fluoro-5-methoxy-1H-indol-3-yl)ethan-1-amine (800 mg, 3.84 mmol) in THF (5.6 mL) was treated with BOC₂O (880 mg, 4.03 mmol) and the mixture was stirred at 25° C. for 2 h. The mixture was concentrated in vacuo, and the residue was purified by preparative TLC (SiO₂, petroleum ether/EtOAc, v/v, 2/1) to give crude tert-butyl (2-(7-fluoro-5-methoxy-1H-indol-3-yl)ethyl)carbamate (500 mg) as a white solid which was used in the subsequent step without further purification.

### Step 8: 2-(7-fluoro-5-methoxy-1H-indol-3-yl)-N-methylethan-1-amine (204)

A solution of crude tert-butyl (2-(7-fluoro-5-methoxy-1H-indol-3-yl)ethyl)carbamate (500 mg) in THF (3.5 mL) at 0° C. was treated with LiAlH₄ (2.50 M in THF, 3.24 mL) and the mixture was stirred at 70° C. for 3 h. The mixture was cooled to 0° C., diluted with THF (70 mL), and quenched with Na₂SO₄·10H₂O (5.00 g). The mixture was stirred at 20° C. for 30 min, filtered, and the filtrate was concentrated in vacuo to give crude 2-(7-fluoro-5-methoxy-1H-indol-3-yl)-N-methylethan-1-amine (400 mg) as a white solid which was used in the subsequent step without further purification.

### Step 9: N-ethyl-2-(7-fluoro-5-methoxy-1H-indol-3-yl)-N-methylethan-1-amine (P-115)

A solution of crude 2-(7-fluoro-5-methoxy-1H-indol-3-yl)-N-methylethan-1-amine (400 mg) in MeOH (4 mL) was treated with NaBH₃CN (226 mg, 3.60 mmol), AcOH (432 mg, 7.20 mmol), and 40% aqueous acetaldehyde (1.08 g, 9.9 mmol), and the mixture was stirred at 20° C. for 3 h. The mixture was adjusted to pH 8 with saturated aqueous Na₂CO₃ solution and concentrated in vacuo. The residue was dissolved in H₂O (22 mL), extracted with EtOAc (12 mL×2), and the combined organic layers were washed with brine (12 mL), dried over Na₂SO₄, filtered, and the filtrate concentrated in vacuo. The residue was purified by preparative HPLC (column: Waters Xbridge 150×25 mm×5 μm; mobile phase: [water (NH₄HCO₃)-ACN]; B: 10-40%, 9 min) to give N-ethyl-2-(7-fluoro-5-methoxy-1H-indol-3-yl)-N-methylethan-1-amine (22.1 mg, 2% over 3 steps) as a yellow solid. ¹H NMR (400 MHZ, MeOD-d₄): δ 7.07 (s, 1H) 6.82 (d, J=2.0 Hz, 1H), 6.53 (dd, J=12.4, 2.0 Hz, 1H), 3.82 (s, 3H), 2.86-2.95 (m, 2H), 2.68-2.77 (m, 2H), 2.61 (q, J=7.2 Hz, 2H), 2.38 (s, 3H) 1.14 (t, J=7.2 Hz, 3H); ¹⁹F NMR (376 MHz, MeOD-d₄): δ −135.5; LCMS (ESI+): m/z 251.1 [M+H]⁺; HPLC purity (254 nm): 96.9%.

### Example 89: N-(2-(7-fluoro-5-methoxy-1H-indol-3-yl)ethyl)-N-methylpropan-2-amine (P-116)

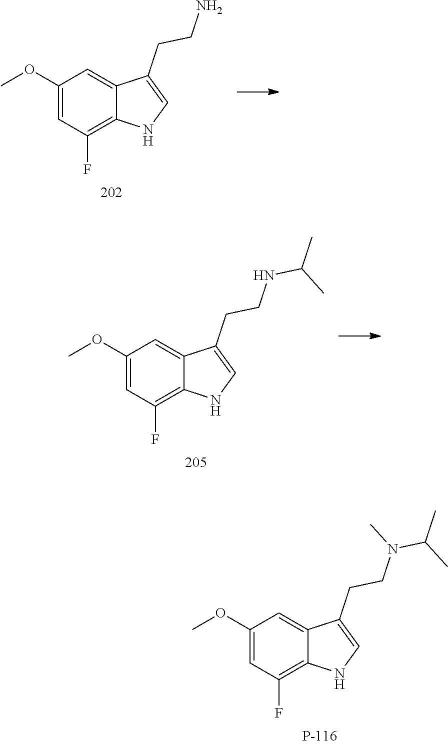 [SMILES: COc1cc(F)c2[nH]cc(CCN)c2c1 ; COc1cc(F)c2[nH]cc(CCNC(C)C)c2c1 ; COc1cc(F)c2[nH]cc(CCN(C)C(C)C)c2c1]

### Step 1: N-(2-(7-fluoro-5-methoxy-1H-indol-3-yl)ethyl)propan-2-amine (205)

To a solution of 2-(7-fluoro-5-methoxy-1H-indol-3-yl)ethan-1-amine (800 mg, 3.84 mmol) in MeOH (8 mL) at 20° C. was added NaBH₃CN (483 mg, 7.68 mmol), AcOH (923 mg, 15.4 mmol), and acetone (1.12 g, 19.2 mmol), and the mixture was stirred at 20° C. for 16 h. The pH of the mixture was adjusted to 8 with saturated aqueous Na₂CO₃, and the organic solvents evaporated in vacuo. The residue was dissolved in H₂O (55 mL), extracted with EtOAc (25 mL×2), and the combined organic layers were washed with brine (25 mL), dried over Na₂SO₄, filtered, and the filtrate concentrated in vacuo to give crude N-(2-(7-fluoro-5-methoxy-1H-indol-3-yl)ethyl)propan-2-amine (210 mg) as a white solid which was used in the subsequent step without further purification.

### Step 2: N-(2-(7-fluoro-5-methoxy-1H-indol-3-yl)ethyl)-N-methylpropan-2-amine (P-116)

To a solution of crude N-(2-(7-fluoro-5-methoxy-1H-indol-3-yl)ethyl)propan-2-amine (210 mg) in MeOH (3 mL) at 20° C. was added NaBH₃CN (105 mg, 1.68 mmol), AcOH (202 mg, 3.36 mmol), 37% w/w aqueous formaldehyde (126 mg, 1.55 mmol), and the mixture was stirred at 20° C. for 16 h. The pH of the mixture was adjusted to 8 with saturated aqueous Na₂CO₃, and the organic solvents were evaporated in vacuo. The residue was dissolved in H₂O (15 mL), extracted with EtOAc (6 mL×2), and the combined organic layers were washed with brine (6 mL), dried over Na₂SO₄, filtered, and the filtrate concentrated in vacuo. The residue was purified by preparative HPLC (column: Waters Xbridge 150×25 mm×5 μm; mobile phase: [water (NH₄HCO₃)-ACN]; B: 15-45%, 10 min) to give N-(2-(7-fluoro-5-methoxy-1H-indol-3-yl)ethyl)-N-methylpropan-2-amine (20.3 mg, 2% over 2 steps) as a yellow solid. ¹H NMR (400 MHz, MeOD-d₄): δ 7.07 (s, 1H), 6.82 (d, J=2.0 Hz, 1H), 6.52 (dd, J=12.6, 2.0 Hz, 1H), 3.81 (s, 3H), 2.98 (sept, J=6.8 Hz, 1H), 2.93-2.83 (m, 2H), 2.77-2.68 (m, 2H), 2.35 (s, 3H), 1.09 (d, J=6.8 Hz, 6H). ¹⁹F NMR (376 MHZ, MeOD-d₄): δ −135.5. LCMS (ESI+): m/z 265.1 [M+H]⁺. HPLC Purity (254 nm): 98.9%.

### Example 90: N, N-diethyl-2-(7-fluoro-5-methoxy-1H-indol-3-yl)ethan-1-amine (P-117)

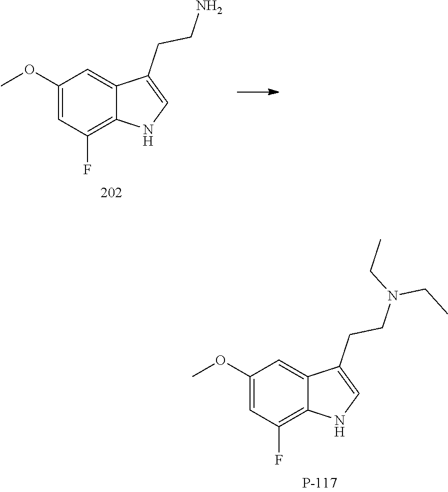 [SMILES: COc1cc(F)c2[nH]cc(CCN)c2c1 ; CCN(CC)CCc1c[nH]c2c(F)cc(OC)cc12]

### Step 1: N,N-diethyl-2-(7-fluoro-5-methoxy-1H-indol-3-yl)ethan-1-amine (P-117)

To a solution of 2-(7-fluoro-5-methoxy-1H-indol-3-yl)ethan-1-amine (800 mg, 3.84 mmol) in MeOH (8 mL) at 20° C. was added NaBH₃CN (483 mg, 7.68 mmol), CH₃CHO (923 mg, 15.4 mmol, 880 μL), and the mixture was stirred for 3 h. The pH of the mixture was adjusted to 8 with saturated aqueous Na₂CO₃, and the organic solvents were evaporated in vacuo. The residue was dissolved in H₂O (55 mL), extracted with EtOAc (25 mL×2), and the combined organic layers were washed with brine (25 mL), dried over Na₂SO₄, filtered, and the filtrate concentrated in vacuo. The residue was purified by preparative HPLC (column: Waters Xbridge 150×25 mm×5 μm; mobile phase: [water (NH₄OH v/v)-ACN]; B: 35-65%, 10 min) to give N,N-diethyl-2-(7-fluoro-5-methoxy-1H-indol-3-yl)ethan-1-amine (21.2 mg, 2% yield) as a yellow solid. ¹H NMR (400 MHZ MeOD-d₄): δ 7.07 (s, 1H), 6.82 (d, J=2.0 Hz, 1H), 6.53 (dd, J=12.6, 2.0 Hz, 1H), 3.82 (s, 3H), 2.93-2.77 (m, 4H), 2.73 (q, J=7.2 Hz, 4H), 1.13 (t, J=7.2 Hz, 6H). ¹⁹F NMR (376 MHZ, MeOD−): δ −135.4. LCMS (ESI+): m/z 265.1 [M+H]⁺. HPLC Purity (254 nm): 97.3%.

### Example 91: N-(2-(7-fluoro-5-methoxy-1H-indol-3-yl)ethyl)-N-isopropylpropan-2-amine (P-118)

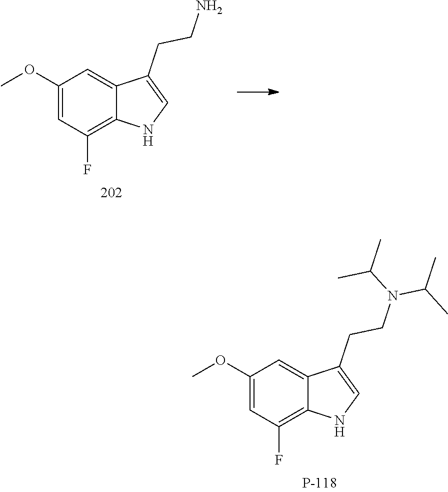 [SMILES: COc1cc(F)c2[nH]cc(CCN)c2c1 ; COc1cc(F)c2[nH]cc(CCN(C(C)C)C(C)C)c2c1]

### Step 1: N-(2-(7-fluoro-5-methoxy-1H-indol-3-yl)ethyl)-N-isopropylpropan-2-amine (P-118)

To a solution of 2-(7-fluoro-5-methoxy-1H-indol-3-yl)ethan-1-amine (800 mg, 3.84 mmol) in MeOH (10 mL) at 20° C. was added NaBH₃CN (2.41 g, 38.4 mmol) and acetone (2.23 g, 38.4 mmol, 2.82 mL) and the mixture was stirred at 20° C. for 3 h. The pH of the mixture was adjusted to 8 with saturated aqueous Na₂CO₃, and the organic solvents were evaporated in vacuo. The residue was dissolved in H₂O (55 mL), extracted with EtOAc (25 mL×2), and the combined organic phases were washed with brine (25 mL), dried over Na₂SO₄, filtered, and concentrated in vacuo. The residue was purified by preparative HPLC (column: Waters Xbridge 150×25 mm×5 μm; mobile phase: [water (NH₄HCO₃)-ACN]; B: 10-40%, 9 min) to give N-(2-(7-fluoro-5-methoxy-1H-indol-3-yl)ethyl)-N-isopropylpropan-2-amine (20.9 mg, 2%) as a yellow solid. ¹H NMR (400 MHZ MeOD-d₄): δ 7.08 (s, 1H), 6.80 (d, J=2.0 Hz, 1H), 6.53 (dd, J=12.6, 2.0 Hz, 1H), 3.81 (s, 3H), 3.30-3.10 (m, 2H), 2.88-2.80 (m, 4H), 1.15 (d, J=6.4 Hz, 12H). ¹⁹F NMR (376 MHZ, MeOD-d₄): δ −135.4. LCMS (ESI+): m/z 293.1 [M+H]⁺. HPLC purity (254 nm): 100%.

Scheme 21: Compounds of general formula (I) can be synthesised from benzimidazolinone starting material 219 following the outlined sequence of steps in Scheme 20 or similar as one skilled in the art may consider. Nitration of 219 can be achieved to yield intermediate 220 which upon rchemoselective reduction allows access to intermediate 221. Hydrazine formation allows access to intermediate 222 which can be used in a Fischer type indole synthesis to afford intermediate 223. Subsequent catalytic dehalogenation provides compounds of general formula (I) (exemplified by P-125). One skilled in the art will recognise that utilising differentially substituted amines during the Fischer cyclisation would allow access to compounds of general formula (I) disclosed herein.

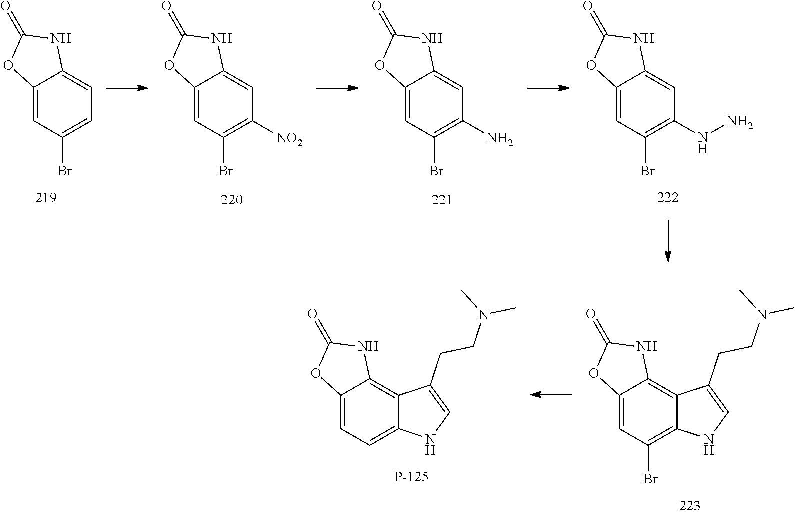

### Example 92: 8-(2-(dimethylamino)ethyl)-1,6-dihydro-2H-oxazolo[4,5-e]indol-2-one (P-125)

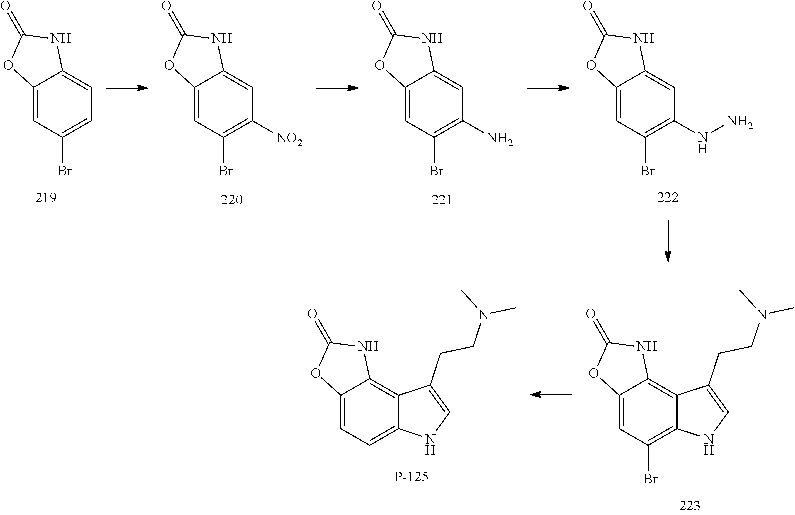 [SMILES: O=c1[nH]c2ccc(Br)cc2o1 ; O=c1[nH]c2cc([N+](=O)[O-])c(Br)cc2o1 ; Nc1cc2[nH]c(=O)oc2cc1Br ; NNc1cc2[nH]c(=O)oc2cc1Br ; CN(C)CCc1c[nH]c2ccc3oc(=O)[nH]c3c12 ; CN(C)CCc1c[nH]c2c(Br)cc3oc(=O)[nH]c3c12]

### Step 1: 6-bromo-5-nitrobenzo[d]oxazol-2 (3H)-one (220)

A solution of 6-bromobenzo[d]oxazol-2 (3H)-one (10.0 g, 46.7 mmol) in H₂SO₄ (100 ml) was stirred at 0° C. for 30 minutes. To this was added KNO₃ (5.57 g, 55.1 mmol) portionwise. The reaction mixture was stirred at 20° C. for 2 h at which point the mixture was poured onto ice water (500 mL) and the precipitate was filtered. The filter cake was washed with H₂O (100 mL), dried and the residue purified by column chromatography (SiO₂, 1-3% THF in CH₂Cl₂) to afford 6-bromo-5-nitrobenzo[d]oxazol-2 (3H)-one (5.17 g, 43%) as a light-yellow solid. ¹H NMR (400 MHZ, DMSO-d₆): δ 12.31 (s, 1H), 7.92 (s, 1H), 7.78 (s, 1H).

### Step 2: 5-amino-6-bromobenzo[d]oxazol-2 (3H)-one (221)

To a solution of 6-bromo-5-nitrobenzo[d]oxazol-2 (3H)-one (4.00 g, 15.4 mmol) in 67% v/v aqueous EtOH (120 mL) was added NH₄Cl (4.13 g, 77.2 mmol). The mixture was heated to 50° C. and iron (4.31 g, 77.2 mmol) was added portionwise. The reaction mixture was stirred at 80° C. for 3 h at which TLC indicated complete consumption of the starting material. The mixture was cooled to 20° C. and diluted with THF (50 mL) and EtOH (50 mL). The mixture was filtered, the filter cake was washed with THF (30 mL), and the combined filtrate concentrated in vacuo. The residue was dissolved in H₂O (30 mL) and the product was extracted with EtOAc (25 mL×2). The combined organic layers were washed with brine (20 mL), dried over anhydrous Na₂SO₄, filtered, and the filtrate concentrated under reduced pressure to afford 5-amino-6-bromobenzo[d]oxazol-2 (3H)-one (3.30 g, 93%) as a brown solid. ¹H NMR (400 MHZ, DMSO-d₆): δ 11.39 (s, 1H), 7.32 (s, 1H), 6.56 (s, 1H), 5.17 (s, 2H).

### Step 3: 6-bromo-5-hydrazineylbenzo[d]oxazol-2 (3H)-one (222)

To a solution of 5-amino-6-bromobenzo[d]oxazol-2 (3H)-one (1.50 g, 6.55 mmol) in 6 M aqueous HCl (15 mL) was added dropwise a solution of NaNO₂ (678 mg, 9.82 mmol) in H₂O (3 mL) at −5 to 5° C. After the reaction mixture was stirred at 0° C. for 30 minutes, a solution of SnCl₂ (2.73 g, 14.4 mmol) dissolved in HCl (6 mL) was added dropwise at −5 to 5° C. The mixture was warmed to ambient temperature and stirring continued for 12 h. The precipitate formed was filtered, washed with H₂O (8 mL×3) and then dried to afford 6-bromo-5-hydrazineylbenzo[d]oxazol-2 (3H)-one as the hydrochloride salt (1.45 g, 79%) which was a yellow solid. ¹H NMR (400 MHZ, DMSO-d₆): δ 12.02 (s, 1H), 10.25 (br s, 3H), 7.79 (br s, 1H), 7.63 (s, 1H), 6.93 (s, 1H).

### Step 4: 5-bromo-8-(2-(dimethylamino)ethyl)-1,6-dihydro-2H-oxazolo[4,5-e]indol-2-one (223)

To a solution of 6-bromo-5-hydrazineylbenzo[d]oxazol-2 (3H)-one hydrochloride (1.45 g, 5.17 mmol) in 4% w/w aqueous H₂SO₄ (14.5 mL) was added (4,4-dimethoxybutyl)dimethylamine (1.15 g, 7.13 mmol) and the mixture was stirred at 100° C. for 2 h. The pH of the mixture was adjusted to 11 with aqueous NH₃ (˜25 mL) and the product was extracted with EtOAc (20 mL×3). The combined organic layers were washed with brine (10 mL), dried over anhydrous Na₂SO₄, filtered, and the filtrate concentrated under reduced pressure. The residue was purified by preparative HPLC (column: Waters Xbridge Prep OBD C18 (150×40 mm×10 μm); mobile phase: [water (NH₃+NH₄HCO₃)-ACN); B: 5-40%, 8 min) to afford 5-bromo-8-(2-(dimethylamino)ethyl)-1,6-dihydro-2H-oxazolo[4,5-e]indol-2-one (100 mg, 5%) as an off-white solid. ¹H NMR (400 MHZ, MeOD-d₄): δ 7.27 (s, 1H), 7.19 (s, 1H), 3.03-2.99 (m, 2H), 2.79-2.74 (m, 2H), 2.46 (s, 6H). LCMS (ESI+): m/z 324.0, 326.0 [M+H]⁺.

### Step 5: 8-(2-(dimethylamino)ethyl)-1,6-dihydro-2H-oxazolo[4,5-e]indol-2-one (P-125)

To a suspension of 10% w/w Pd/C (14.0 mg, 13.2 μmol) in MeOH (2.10 mL) was added 5-bromo-8-(2-(dimethylamino)ethyl)-1,6-dihydro-2H-oxazolo[4,5-e]indol-2-one (70.0 mg, 216 μmol). The reaction mixture was stirred at 20° C. for 8 h under H₂ atmosphere at 50 PSI. The reaction mixture was filtered through a pad of celite and the filtrate concentrated under reduced pressure. The residue was purified by preparative HPLC (column: Waters Xbridge Prep OBD C18 (150×40 mm×10 μm); mobile phase: [water (NH₃+NH₄HCO₃)-ACN); B: 1-30%, 8 min) to afford 8-(2-(dimethylamino)ethyl)-1,6-dihydro-2H-oxazolo[4,5-e]indol-2-one (20.9 mg, 39%) as a white solid. ¹H NMR (400 MHZ, MeOD-d₄): δ 7.10-7.07 (m, 2H), 7.01 (d, J=8.8 Hz, 1H), 3.00-2.94 (m, 2H), 2.64-2.58 (m, 2H), 2.39 (s, 6H). LCMS (ESI+): m/z 246.1 [M+H]⁺. HPLC Purity (220 nm): 97.9%.

Scheme 22: Compounds of general formula (I) can be synthesised from 5-hydroxytryptamine starting material 224 following the outlined sequence of steps in Scheme 22 or similar as one skilled in the art may consider. Addition of a tert-butoxy carbonyl protecting group to starting material 224 provides N-protected intermediate 225. Addition of methylamine under oxidative conditions allows access to oxazoloindole intermediate 226. Subsequent treatment with TFA allows access to primary amine intermediate 227 which permits N-alkylation using an appropriate aldehyde and reducing agent to provide compounds of general formula (I) (exemplified by P-126). One skilled in the art will recognise that utilising differentially substituted aldehydes would allow access to compounds of general formula (I) disclosed herein.

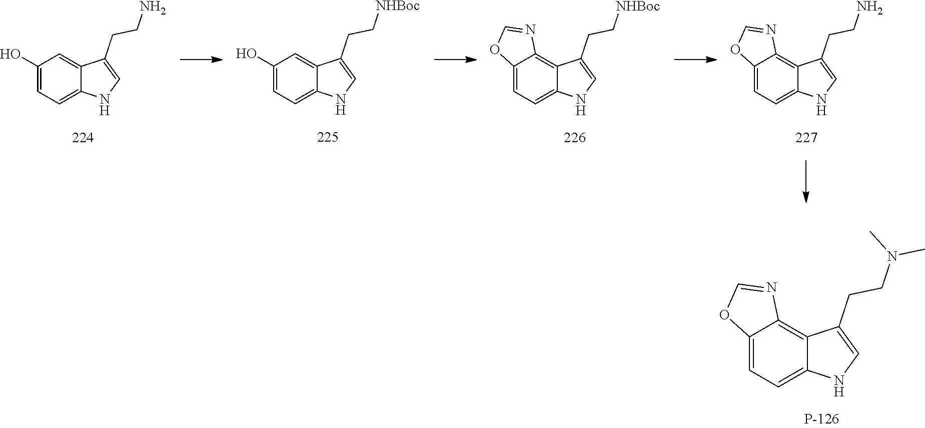

### Example 93: N,N-dimethyl-2-(6H-oxazolo[4,5-e]indol-8-yl)ethan-1-amine (P-126)

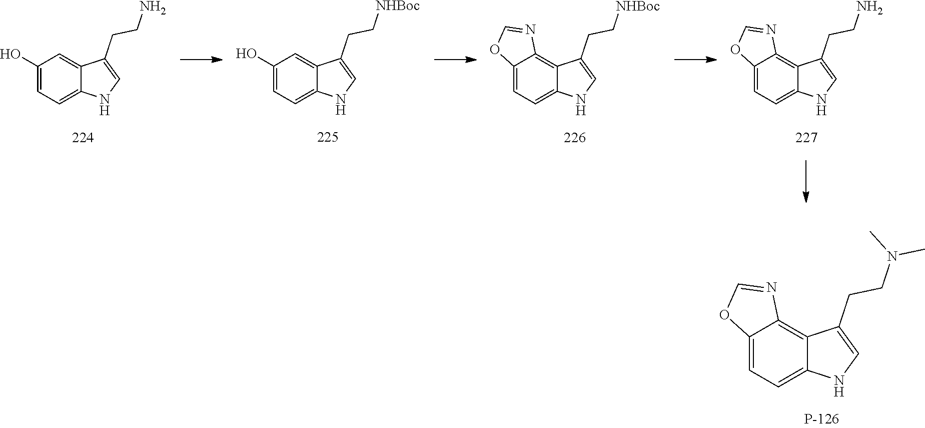 [SMILES: NCCc1c[nH]c2ccc(O)cc12 ; CC(C)(C)OC(=O)NCCc1c[nH]c2ccc(O)cc12 ; CC(C)(C)OC(=O)NCCc1c[nH]c2ccc3ocnc3c12 ; NCCc1c[nH]c2ccc3ocnc3c12 ; CN(C)CCc1c[nH]c2ccc3ocnc3c12]

### Step 1: tert-butyl (2-(5-hydroxy-1H-indol-3-yl)ethyl)carbamate (225)

A solution of 3-(2-aminoethyl)-1H-indol-5-ol hydrochroride (10.7 g, 50.3 mmol) in THF (40 mL), H₂O (20 mL), and Et₃N (15.3 g, 151 mmol) was stirred for 10 min before addition of Boc₂O (12.1 g, 55.3 mmol) and stirred at ambient temperature for 4 h. The reaction was diluted with H₂O (100 mL), and extracted with EtOAc (50 mL×3). The combined organic layers were washed with brine (35 mL), dried over anhydrous Na₂SO₄, and concentrated in vacuo. The residue was purified by column chromatography (SiO₂, petroleum ether: EtOAc-49:1 to 19:1, v/v) to afford tert-butyl (2-(5-hydroxy-1H-indol-3-yl)ethyl)carbamate (8.70 g, 63%) as a yellow oil. ¹H NMR (400 MHZ, CDCl₃): δ 7.93 (s, 1H), 7.22 (d, J=8.8 Hz, 1H), 7.01 (m, 2H), 6.78-6.81 (m, 1H), 4.64 (s, 1H), 3.44-3.45 (m, 2H), 2.89 (t, J=6.4 Hz, 2H), 1.45 (s, 9H).

### Step 2: tert-butyl (2-(6H-oxazolo[4,5-e]indol-8-yl)ethyl)carbamate (226)

A solution of tert-butyl (2-(5-hydroxy-1H-indol-3-yl)ethyl)carbamate (5.6 g, 20.3 mmol) in THF (39 mL) was treated with 2 M MeNH₂ in THF (30.4 mL, 60.8 mmol) and MnO₂ (17.6 g, 203 mmol) under N₂ and then stirred at room temperature for 16 h. The reaction was filtered and the filter cake washed with THF (50 mL). The combined filtrate was concentrated in vacuo and the residue was purified by column chromatography (SiO₂, petroleum ether: EtOAc-19:1 to 9:1, v/v) to afford tert-butyl (2-(6H-oxazolo[4,5-e]indol-8-yl)ethyl)carbamate (2.00 g, 33%) as a colourless oil. 1H NMR (400 MHZ, CDCl₃): δ 8.40 (br. s, 1H), 8.15 (s, 1H), 7.36-7.43 (m, 2H), 7.15 (s, 1H), 5.19 (br. s, 1H), 3.61-3.66 (m, 2H), 3.22-3.26 (m, 2H), 1.34 (s, 9H).

### Step 3: 2-(6H-oxazolo[4,5-e]indol-8-yl)ethan-1-amine (227)

A solution of tert-butyl (2-(6H-oxazolo[4,5-e]indol-8-yl)ethyl)carbamate (1.00 g, 3.32 mmol) in EtOAc (7 mL) was treated with 4 M aqueous HCl in EtOAc (30 mL) and stirred at ambient temperature for 3 h. The precipitate was filtered and the solid was washed with EtOAc (3 mL) and Et₂O (5 mL). The dried solid was purified by preparative HPLC (column: Waters Xbridge Prep OBD C18 (150*40 mm*10 μm); mobile phase: [water (NH₃H₂O+NH₄HCO₃)-ACN]; B: 1-45%, 8 min) to afford 2-(6H-oxazolo[4,5-e]indol-8-yl)ethan-1-amine (220 mg, 33%), which was a brown solid. 1H NMR (400 MHZ, MeOD-d₄): δ 8.42 (s, 1H), 7.37-7.44 (m, 2H), 7.22 (s, 1H), 3.14-3.17 (m, 2H), 3.05-3.09 (m, 2H).

### Step 4: N,N-dimethyl-2-(6H-oxazolo[4,5-e]indol-8-yl)ethan-1-amine (P-126)

A solution of 2-(6H-oxazolo[4,5-e]indol-8-yl)ethan-1-amine (200 mg, 0.99 mmol) in MeOH (4 mL) was treated with AcOH (239 mg, 3.98 mmol), NaBH₃CN (125 mg, 1.99 mmol), and 37% w/w formaldehyde (202 mg, 2.48 mmol) and stirred at ambient temperature for 12 h. Upon completion, the pH was adjusted to 7-8 with saturated aqueous Na₂CO₃ solution and was then extracted with CH₂Cl₂ (10 mL×3). The combined organic layers were washed with brine (10 mL), dried over anhydrous Na₂SO₄ and concentrated in vacuo. The residue was purified by preparative HPLC (column: Phenomenex Luna C18 (150×30 mm×5 μm); mobile phase: [water (TFA)-ACN]; B: 1-30%, 8 min), the pH of the collected fraction was adjusted to 9-10 with aqueous NH₃ and extracted with CH₂Cl₂ (10 mL×2). The combined organic layers were washed with brine (3 mL), dried over Na₂SO₄, filtered, and the filtrate concentrated to afford N,N-dimethyl-2-(6H-oxazolo[4,5-e]indol-8-yl)ethan-1-amine (20.5 mg, 9%) as a brown solid. ¹H NMR (400 MHZ, MeOD-d₄): δ 8.36 (s, 1H), 7.35-7.42 (m, 2H), 7.20 (s, 1H), 3.23 (t, J=8.4 Hz, 2H), 2.90 (t, J=8.4 Hz, 2H), 2.40 (s, 6H). LCMS (ESI+): m/z 230.1 [M+H]⁺. HPLC Purity (220 nm): 97.7%.

### Example 94: N-ethyl-N-methyl-2-(6H-oxazolo[4,5-e]indol-8-yl)ethan-1-amine (P-127)

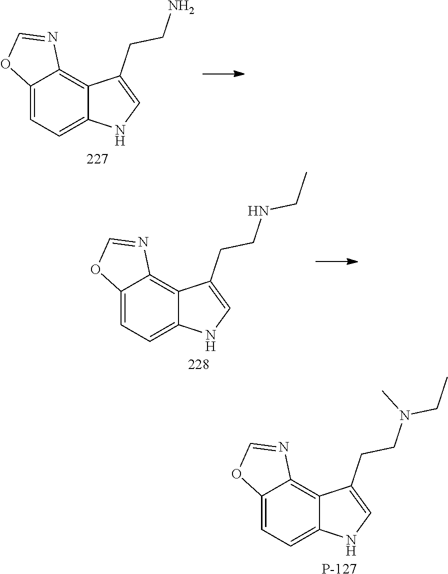

### Step 1: N-ethyl-2-(6H-oxazolo[4,5-e]indol-8-yl)ethan-1-amine (228)

To a mixture of 2-(6H-oxazolo[4,5-e]indol-8-yl)ethan-1-amine (600 mg, 2.98 mmol) in MeOH (12 mL) was added AcOH (215 mg, 3.58 mmol) and NaBH₃CN (225 mg, 3.58 mmol) and 40% w/w acetaldehyde (394 mg, 3.58 mmol) which was stirred at 50° C. for 2 h. The reaction was quenched with H₂O (20 mL) and extracted with CH₂Cl₂ (10 mL×3). The combined organic layers were washed with brine (10 mL), dried over Na₂SO₄ and concentrated under reduced pressure to provide crude N-ethyl-2-(6H-oxazolo[4,5-e]indol-8-yl)ethan-1-amine (400 mg) as brown oil which was used in the subsequent step without further purification.

### Step 2: N-ethyl-N-methyl-2-(6H-oxazolo[4,5-e]indol-8-yl)ethan-1-amine (P-127)

To a mixture of crude N-ethyl-2-(6H-oxazolo[4,5-e]indol-8-yl)ethan-1-amine (400 mg) in MeOH (8 mL) was added AcOH (210 mg, 3.49 mmol) and NaBH₃CN (274 mg, 4.36 mmol) and 37% w/w formaldehyde (383 mg, 4.72 mmol) which was stirred at 50° C. for 2 h. The mixture was quenched with H₂O (20 mL) and then extracted with CH₂Cl₂ (10 mL×3). The combined organic layers were washed with brine (10 mL), dried over Na₂SO₄ and concentrated under reduced pressure. The crude product was purified by preparative HPLC (column: Waters Xbridge (150*25 mm*5 μm); mobile phase: [water (NH₄HCO₃)-ACN]; B: 12-42%, 9 min) to provide N-ethyl-N-methyl-2-(6H-oxazolo[4,5-e]indol-8-yl)ethan-1-amine (21.7 mg, 3% over 2 steps) as brown gum. 1H NMR (400 MHZ, CDCl₃): δ 8.40 (br. s, 1H), 8.13 (s, 1H), 7.41-7.35 (m, 2H), 7.20 (d, J=2.4 Hz, 1H), 3.34-3.30 (m, 2H), 3.10-3.06 (m, 2H), 2.76 (q, J=7.2 Hz, 2H), 2.16 (s, 3H), 1.19 (t, J=7.2 Hz, 3H). LCMS (ESI+): m/z 244.2 [M+H]⁺. HPLC Purity (220 nm): 96.7%.

### Example 95: N-(2-(6H-oxazolo[4,5-e]indol-8-yl)ethyl)-N-methylpropan-2-amine (P-128)

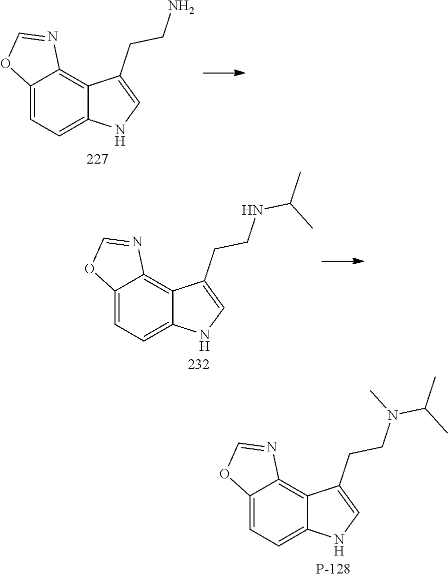

### Step 1: N-(2-(6H-oxazolo[4,5-e]indol-8-yl)ethyl)propan-2-amine (232)

To a mixture of 2-(6H-oxazolo[4,5-e]indol-8-yl)ethan-1-amine (0.50 g, 2.48 mmol) in MeOH (10 mL) was added AcOH (298 mg, 4.97 mmol), NaBH₃CN (187 mg, 2.98 mmol) and acetone (289 mg, 4.97 mmol) and the mixture was stirred at 50° C. for 2 h. The mixture was quenched with H₂O (20 mL) and extracted with CH₂Cl₂ (10 mL×3). The combined organic layers were washed with brine (10 mL), dried over Na₂SO₄ and concentrated under reduced pressure to provide crude N-(2-(6H-oxazolo[4,5-e]indol-8-yl)ethyl)propan-2-amine (700 mg) as a brown solid which was used in the subsequent step without further purification.

### Step 2: N-(2-(6H-oxazolo[4,5-e]indol-8-yl)ethyl)-N-methylpropan-2-amine (P-128)

To a mixture of crude N-(2-(6H-oxazolo[4,5-e]indol-8-yl)ethyl)propan-2-amine (700 mg) in MeOH (14 mL) was added AcOH (691 mg, 11.5 mmol), NaBH₃CN (362 mg, 5.76 mmol) and 37% w/w formaldehyde (584 mg, 7.19 mmol) and the mixture was stirred at 50° C. for 2 h. The mixture was quenched with H₂O (20 mL) and extracted with CH₂Cl₂ (10 mL×3). The combined organic layers were washed with brine (10 mL), dried over Na₂SO₄ and concentrated under reduced pressure. The residue was purified by preparative HPLC (column: Waters Xbridge Prep OBD C18 (150×40 mm×10 μm); mobile phase: [water (NH₄HCO₃)-ACN]; B: 12-32%, 15 min) to provide N-(2-(6H-oxazolo[4,5-e]indol-8-yl)ethyl)-N-methylpropan-2-amine (193 mg, 30% over 2 steps) as gray solid. ¹H NMR (400 MHZ, CDCl₃): δ 8.50 (br. s, 1H), 8.13 (s, 1H), 7.41 (d, J=8.8 Hz, 1H), 7.31 (d, J=8.8 Hz, 1H), 7.23 (d, J=2.2 Hz, 1H), 3.29-3.32 (m, 2H), 3.09-3.17 (m, 3H), 2.50 (s, 3H), 1.12 (d, J=6.6 Hz, 6H). LCMS (ESI+): m/z 258.2 [M+H]⁺. HPLC Purity (220 nm): 98.5%.

### Example 96: N,N-diethyl-2-(6H-oxazolo[4,5-e]indol-8-yl)ethan-1-amine (P-129)

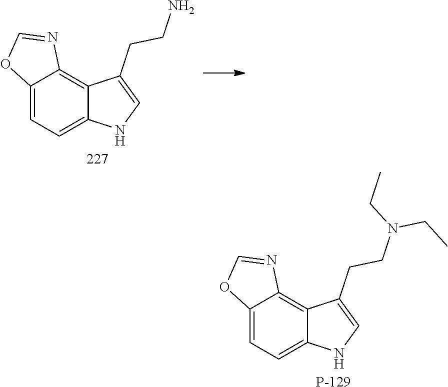

### Step 1: N,N-diethyl-2-(6H-oxazolo[4,5-e]indol-8-yl)ethan-1-amine (P-129)

To a mixture of 2-(6H-oxazolo[4,5-e]indol-8-yl)ethan-1-amine (100 mg, 0.50 mmol) in MeOH (2 mL) was added AcOH (101 mg, 1.68 mmol), 40% w/w acetaldehyde (116 mg, 1.05 mmol) and NaBH₃CN (52.9 mg, 0.84 mmol) and the mixture was stirred at 50° C. for 2 h. The mixture was quenched with H₂O (30 mL) and extracted with CH₂Cl₂ (10 mL×3). The combined organic layers were washed with brine (10 mL), dried over Na₂SO₄ and concentrated under reduced pressure. The crude product was purified by preparative HPLC (column: Waters Xbridge (150×25 mm×5 μm); mobile phase: [water (NH₄HCO₃)-ACN]; B: 10-40%, 9 min) to provide N,N-diethyl-2-(6H-oxazolo[4,5-e]indol-8-yl)ethan-1-amine (30.0 mg, 23%) as a brown solid. ¹H NMR (400 MHZ, CDCl₃): δ 8.49 (br. s, 1H), 8.12 (s, 1H), 7.48-7.33 (m, 2H), 3.39-3.29 (m, 4H), 2.99 (q, J=7.2 Hz, 4H), 1.28 (t, J=7.2 Hz, 6H). LCMS (ESI+): m/z 258.1 [M+H]⁺. HPLC Purity (220 nm): 97.9%.

### Example 97: N-(2-(6H-oxazolo[4,5-e]indol-8-yl)ethyl)-N-isopropylpropan-2-amine (P-130)

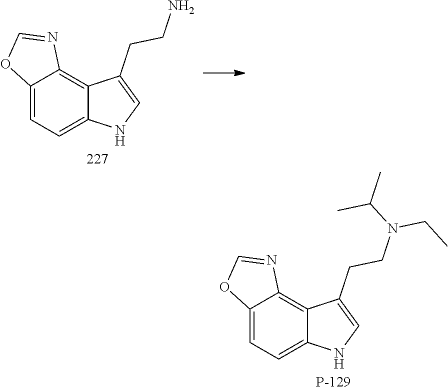

### Step 1: N-(2-(6H-oxazolo[4,5-e]indol-8-yl)ethyl)-N-isopropylpropan-2-amine (P-130)

To a mixture of 2-(6H-oxazolo[4,5-e]indol-8-yl)ethan-1-amine (600 mg, 2.98 mmol) in MeOH (12 mL) was added AcOH (303 mg, 5.05 mmol), acetone (1.47 g, 25.3 mmol) and NaBH₃CN (1.59 g, 25.3 mmol) and the mixture was stirred at 50° C. for 2 h. The mixture was quenched with H₂O (30 mL) and extracted with CH₂Cl₂ (10 mL×3). The combined organic layers were washed with brine (10 mL), dried over Na₂SO₄ and concentrated under reduced pressure. The crude product was purified by preparative HPLC (column: Waters Xbridge Prep OBD C18 (150×40 mm×10 μm); mobile phase: [water (NH₄HCO₃)-ACN]; B: 8-38%, 15 min) to provide N-(2-(6H-oxazolo[4,5-e]indol-8-yl)ethyl)-N-isopropylpropan-2-amine (291 mg, 34%) as a brown solid. ¹H NMR (400 MHz, MeOD-d₄): δ 8.36 (s, 1H), 7.45-7.30 (m, 2H), 7.24 (s, 1H), 3.36-3.46 (m, 2H), 3.16-3.24 (m, 4H), 1.27 (t, J=6.6 Hz, 6H). LCMS (ESI+): m/z 286.2 [M+H]⁺. HPLC Purity (220 nm): 99.3%.

### Example 98: N,N-dimethyl-2-(2-methyl-6H-oxazolo[4,5-e]indol-8-yl)ethan-1-amine (P-131)

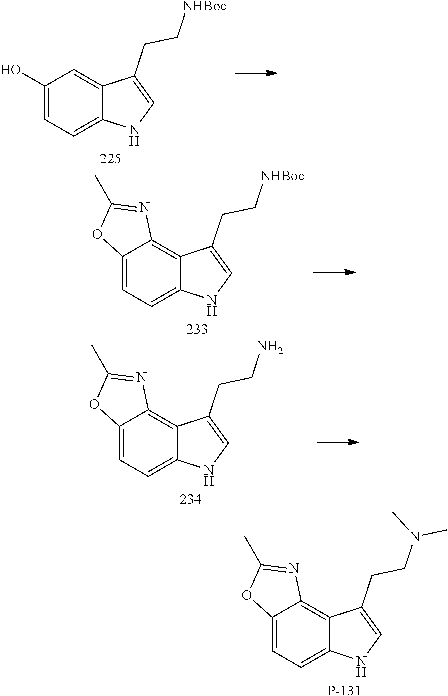

### Step 1: tert-butyl (2-(2-methyl-6H-oxazolo[4,5-e]indol-8-yl)ethyl)carbamate (233)

A solution of tert-butyl (2-(5-hydroxy-1H-indol-3-yl)ethyl)carbamate (2.57 g, 9.3 mmol) in THF (51 mL) was treated with EtNH₂ (2.10 g, 46.5 mmol) and MnO₂ (8.09 g, 93.0 mmol) under N₂ and then stirred at room temperature for 16 h. The precipitate was filtered and washed with THF (80 mL). The combined filtrate was concentrated in vacuo and the residue was purified by column chromatography (SiO₂, petroleum ether: EtOAc-97:3 to 92:8, v/v) to afford crude tert-butyl (2-(2-methyl-6H-oxazolo[4,5-e]indol-8-yl)ethyl)carbamate (800 mg) as a brown solid which was used in the next step without further purification. LCMS (ESI+): m/z 316.2 [M+H]⁺.

### Step 2: 2-(2-methyl-6H-oxazolo[4,5-e]indol-8-yl)ethan-1-amine (234)

A solution of crude tert-butyl (2-(2-methyl-6H-oxazolo[4,5-e]indol-8-yl)ethyl)carbamate (800 mg) in EtOAc (4 mL) was treated with 4 M HCl in EtOAc (10 mL) and stirred at room temperature for 3 h. Upon completion, the precipitate was filtered and the solid washed with EtOAc (3 mL) and petroleum ether (5 mL). The dried solid was then purified by preparative HPLC (column: Waters Xbridge Prep OBD C18 (150×40 mm×10 μm); mobile phase: [water (aq. NH₃+NH₄HCO₃)-ACN]; B: 1-40%, 8 min) to afford 2-(2-methyl-6H-oxazolo[4,5-e]indol-8-yl)ethan-1-amine (220 mg, 11% over 2 steps) as an off-white solid. ¹H NMR (400 MHz, MeOD-d₄): δ 7.28-7.34 (m, 2H), 7.18 (s, 1H), 3.11-3.15 (m, 2H), 3.02-3.05 (m, 2H), 2.67 (s, 3H). LCMS (ESI+): m/z 244.1 [M+H]⁺. HPLC Purity (220 nm): 97.8%.

### Step 3: N,N-dimethyl-2-(2-methyl-6H-oxazolo[4,5-e]indol-8-yl)ethan-1-amine (P-131)

A solution of 2-(2-methyl-6H-oxazolo[4,5-e]indol-8-yl)ethan-1-amine (210 mg, 0.98 mmol) in MeOH (5 mL) was treated with AcOH (234 mg, 3.90 mmol), NaBH₃CN (123 mg, 1.95 mmol), and 37% w/w aq. formaldehyde (198 mg, 2.44 mmol) and was stirred at room temperature for 12 h. Upon completion, the pH was adjusted to 7-8 with saturated aqueous Na₂CO₃ solution and then extracted with CH₂Cl₂ (10 mL×3). The combined organic layers were washed with brine (10 mL), dried over anhydrous Na₂SO₄ and concentrated in vacuo. The crude product was purified by preparative HPLC (column: Waters Xbridge BEH C18 (100×30 mm×10 μm); mobile phase: [water (NH₄HCO₃)-ACN]; B: 1-40%, 8 min) to afford N,N-dimethyl-2-(2-methyl-6H-oxazolo[4,5-e]indol-8-yl)ethan-1-amine (53.3 mg, 22%) as an off-white solid. ¹H NMR (400 MHZ, MeOD-d₄): δ 7.26-7.32 (m, 2H), 7.16 (s, 1H), 3.21 (t, J=8.0 Hz, 2H), 2.88 (t, J=8.0 Hz, 2H), 2.65 (s, 3H), 2.40 (s, 6H). LCMS (ESI+) m/z 244.1 [M+H]⁺.

### Example 99: N-ethyl-N-methyl-2-(2-methyl-6H-oxazolo[4,5-e]indol-8-yl)ethan-1-amine (P-132)

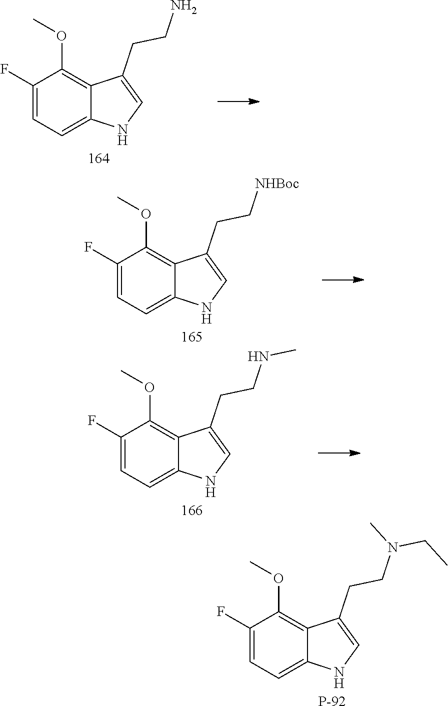

### Step 1: 3-(2-(methylamino)ethyl)-1H-indol-5-ol (235)

To a solution of tert-butyl (2-(5-hydroxy-1H-indol-3-yl)ethyl)carbamate (5.00 g, 18.1 mmol) in THF (35 mL) was added 2.5 M LiAlH₄ in THF (36.1 mL. 90.3 mmol) at 0° C. and the mixture was stirred at 70° C. for 1 h. The reaction mixture was quenched with H₂O (3.5 mL) and 15% NaOH (3.5 mL) and then again H₂O (10.5 mL). The mixture was extracted with EtOAc (20 mL×3) and the combined organic layers were washed with brine (10 mL), dried over anhydrous Na₂SO₄ and concentrated under reduced pressure to give crude 3-(2-(methylamino)ethyl)-1H-indol-5-ol (1.40 g) as brown oil which was used in the subsequent step without further purification. LCMS (ESI+): m/z 191 [M+H]⁺.

### Step 2: 3-(2-(ethyl(methyl)amino)ethyl)-1H-indol-5-ol (236)

To a solution of 3-(2-(methylamino)ethyl)-1H-indol-5-ol (1.40 g, 7.36 mmol) in MeOH (35 mL) was added NaBH₃CN (1.16 g, 18.5 mmol), AcOH (1.48 g, 24.6 mmol) and acetaldehyde (1.57 mL, 27.7 mmol) and the mixture was stirred at 25° C. for 16 h. The pH was adjusted 7-8 with saturated aqueous Na₂CO₃ solution and then extracted with CH₂Cl₂ (30 mL×4). The combined organic layers were washed with brine (30 mL×4), dried over anhydrous Na₂SO₄ and concentrated under reduced pressure to give crude 3-(2-(ethyl(methyl)amino)ethyl)-1H-indol-5-ol (1.0 g) as a yellow solid which was used in the subsequent step without further purification. LCMS (ESI+): m/z 219.3 [M+H]⁺.

### Step 3: N-ethyl-N-methyl-2-(2-methyl-6H-oxazolo[4,5-e]indol-8-yl)ethan-1-amine (P-132)

To a solution of crude 3-(2-(ethyl(methyl)amino)ethyl)-1H-indol-5-ol (1.00 g) in THF (20 mL) was added MnO₂ (3.98 g, 45.8 mmol) and EtNH₂ (1.03 g, 22.8 mmol) under N₂ and the reaction was stirred at 25° C. for 16 h. The mixture was then filtered, and the filter cake was washed with THF (200 mL). The combined filtrate was concentrated under reduced pressure and the residue was purified by preparative HPLC (column: Phenomenex luna C18 (150×40 mm×15 μm); mobile phase: [water (formic acid)-ACN]; B: 2-32%, 11 min) to provide N-ethyl-N-methyl-2-(2-methyl-6H-oxazolo[4,5-e]indol-8-yl)ethan-1-amine (30.0 mg, 1% over 3 steps) as a yellow solid. ¹H NMR (400 MHz, MeOD-d₄): δ 7.26-7.32 (m, 2H), 7.17 (s, 1H), 3.20-3.24 (m, 2H), 2.98-3.02 (m, 2H), 2.70 (q, J=7.2 Hz, 2H), 2.65 (s, 3H), 2.46 (s, 3H), 1.17 (t, J=7.2 Hz, 3H). LCMS (ESI+): m/z 258.1 [M+H]⁺. HPLC Purity (220 nm): 97.6%.

### Example 100: N-ethyl-N-methyl-2-(2-methyl-6H-oxazolo[4,5-e]indol-8-yl)ethan-1-amine (P-133)

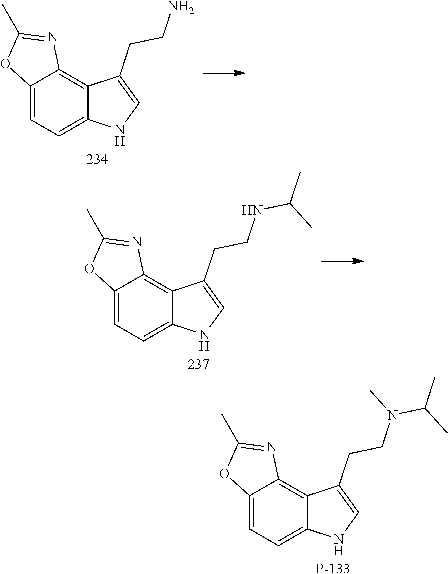

### Step 1: N-(2-(2-methyl-6H-oxazolo[4,5-e]indol-8-yl)ethyl)propan-2-amine (237)

To a solution of 2-(2-methyl-6H-oxazolo[4,5-e]indol-8-yl)ethan-1-amine (600 mg, 2.38 mmol) in MeOH (15 mL) was added NaBH₃CN (7.49 g, 119 mmol), acetone (6.91 g, 119 mmol) and AcOH (573 mg, 9.53 mmol) and then stirred at 25° C. for 12 h. The reaction was then diluted with saturated aqueous Na₂CO₃ solution until the pH was 7-8 at which point the mixture was extracted with CH₂Cl₂ (10 mL×3). The combined organic layers were washed with brine (30 mL), dried over Na₂SO₄, filtered, and the filtrate concentrated under reduced pressure to provide crude N-(2-(2-methyl-6H-oxazolo[4,5-e]indol-8-yl)ethyl)propan-2-amine (600 mg) as brown oil which was used in the subsequent step without further purification.

### Step 2: N-methyl-N-(2-(2-methyl-6H-oxazolo[4,5-e]indol-8-yl)ethyl)propan-2-amine (P-133)

To a solution of crude N-(2-(2-methyl-6H-oxazolo[4,5-e]indol-8-yl)ethyl)propan-2-amine (300 mg, 1.17 mmol) in MeOH (7.5 mL) was added NaBH₃CN (366 mg, 5.82 mmol), AcOH (280 mg, 4.66 mmol) and 37% w/w aqueous formaldehyde (4.73 g, 58.2 mmol) and the mixture was stirred at 25° C. for 12 h. The reaction was quenched by addition of H₂O (3 mL) and was then extracted with EtOAc (15 mL×3). The combined organic layers were washed with brine (15 mL), dried over anhydrous Na₂SO₄ and concentrated under reduced pressure. The residue was purified by preparative HPLC (column: Waters Xbridge (150×25 mm×5 μm); mobile phase: [water (NH₄HCO₃)-ACN]; B: 18-48%, 9 min) to provide N-methyl-N-(2-(2-methyl-6H-oxazolo[4,5-e]indol-8-yl)ethyl)propan-2-amine (19 mg, 6.9%) as a yellow solid. ¹H NMR (400 MHZ, MeOD-d₄): δ 7.33-7.38 (m, 2H), 7.30 (s, 1H), 3.63 (sept, J=6.5 Hz, 1H), 3.51-3.55 (m, 2H), 3.36-3.39 (m, 2H), 2.85 (s, 3H), 2.68 (s, 3H), 1.32 (d, J=6.8 Hz, 6H). LCMS (ESI+): m/z 272.1 [M+H]⁺. HPLC Purity (220 nm): 97.8%.

### Example 101: N,N-diethyl-2-(2-methyl-6H-oxazolo[4,5-e]indol-8-yl)ethan-1-amine (P-134)

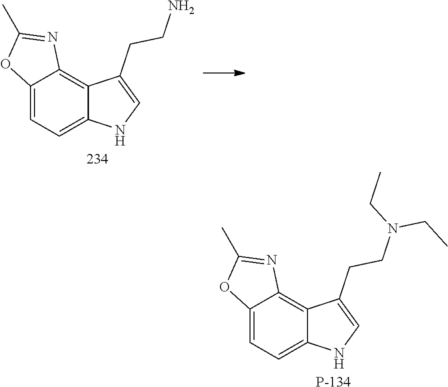

### Step 1: N,N-diethyl-2-(2-methyl-6H-oxazolo[4,5-e]indol-8-yl)ethan-1-amine (P-134)

To a solution 2-(2-methyl-6H-oxazolo[4,5-e]indol-8-yl)ethan-1-amine hydrochloride (200 mg, 0.79 mmol) in MeOH (5 mL) was added NaBH₃CN (2.37 g, 37.7 mmol), AcOH (190 mg, 3.16 mmol) and 40% w/w acetaldehyde (2.05 g, 18.6 mmol) and the mixture was stirred at 25° C. for 12 h. The reaction mixture was adjusted to pH 7-8 with saturated aqueous Na₂CO₃ solution and then extracted with CH₂Cl₂ (10 mL×3). The combined organic layers were washed with brine (10 mL×3), dried over anhydrous Na₂SO₄ and concentrated under reduced pressure. The residue was purified by preparative HPLC (column: Waters Xbridge (150×25 mm×5 μm); mobile phase: [water (NH₄HCO₃)-ACN]; B: 20-50%, 9 min) to provide N,N-diethyl-2-(2-methyl-6H-oxazolo[4,5-e]indol-8-yl)ethan-1-amine (30 mg, 14%) as a white solid. 1H NMR (400 MHZ, MeOD-d₄): δ 7.28-7.33 (m, 2H), 7.21 (s, 1H), 3.20-3.26 (m, 4H), 2.96 (q, J=7.2 Hz, 4H), 2.65 (s, 3H), 1.23 (t, J=7.2 Hz, 6H). LCMS (ESI+): m/z 272.0 [M+H]⁺. HPLC Purity (254 nm): 97.6%.

### Example 102: N-isopropyl-N-(2-(2-methyl-6H-oxazolo[4,5-e]indol-8-yl)ethyl)propan-2-amine (P-135)

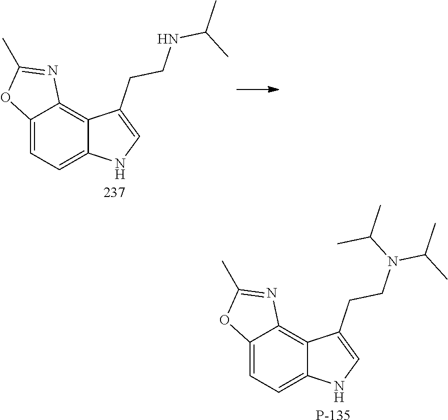

### Step 1: N-isopropyl-N-(2-(2-methyl-6H-oxazolo[4,5-e]indol-8-yl)ethyl)propan-2-amine (P-135)

To a solution of crude N-(2-(2-methyl-6H-oxazolo[4,5-e]indol-8-yl)ethyl)propan-2-amine (300 mg) in MeOH (7.5 mL) was added acetone (5.18 mL, 69.9 mmol), AcOH (334 mg, 5.56 mmol) and NaBH₃CN (4.4 g, 69.9 mmol) and the mixture was stirred at 40° C. for 96 h. The pH was adjusted 7-8 with saturated aqueous Na₂CO₃ solution and then extracted with CH₂Cl₂ (10 mL×3). The combined organic layers were washed with brine (10 mL×3), dried over anhydrous Na₂SO₄ and concentrated under reduced pressure. The residue was purified by preparative HPLC (column: Waters Xbridge (150×25 mm×5 μm); mobile phase: [water (NH₄HCO₃)-ACN]; B: 20-50%, 9 min) to afford N-isopropyl-N-(2-(2-methyl-6H-oxazolo[4,5-e]indol-8-yl)ethyl)propan-2-amine (60 mg, 14% over 2 steps) obtained as white solid. ¹H NMR (400 MHZ, MeOD-d₄): δ 7.37-7.20 (m, 3H), 3.79 (sept, J=6.8 Hz, 2H), 3.55-3.59 (m, 2H), 3.33-3.37 (m, 2H), 2.67 (s, 3H), 1.45 (d, J=6.6 Hz, 12H). LCMS (ESI+): m/z 300.3 [M+H]⁺. HPLC Purity (220 nm): 96.7%.

Scheme 23: Compounds of general formula (I) can be synthesised from the appropriately substituted indole following the outlined sequence of steps in Scheme 23 or similar as one skilled in the art may consider. Substituted indole cores could be condensed with dimethylamino-2-nitroethylene to give nitrovinyl indoles. Reduction of such intermediates proved viable in accessing unsubstituted ethyl amine analogues that could subsequently undergo reductive alkylation to access compounds of general formula (I) (exemplified by P-142). One skilled in the art will recognise that protecting the amine with a suitable protecting group such as benzyl, followed by alkylation, subsequent deprotection and a second alkylation would allow access to differentially alkylated compounds of general formula (I) disclosed herein.

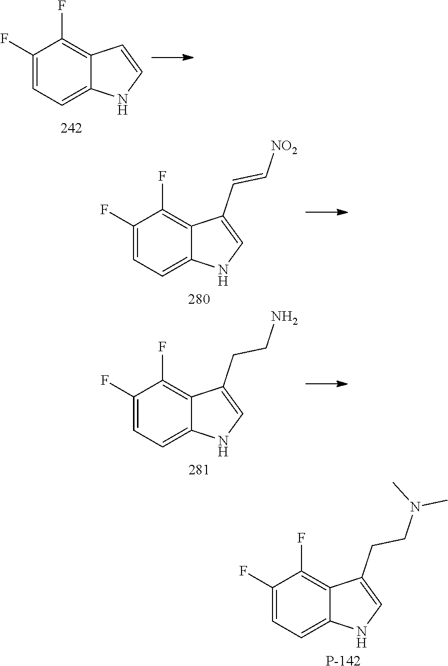

### Example 103:2-(4,5-difluoro-1H-indol-3-yl)-N,N-dimethylethan-1-amine (P-142)

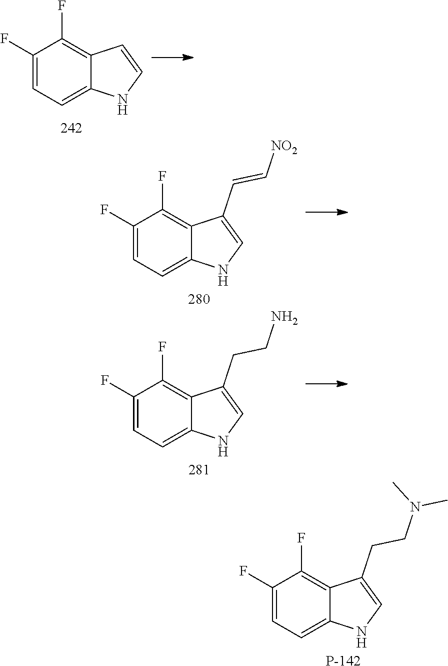

### Step 1: (E)-4,5-difluoro-3-(2-nitrovinyl)-1H-indole (280)

To a solution of 4,5-difluoro-1H-indole (0.50 g, 3.27 mmol) in TFA (3.50 mL) was added (E)-N,N-dimethyl-2-nitroethen-1-amine (379 mg, 3.27 mmol) and the mixture was stirred at 25° C. for 1 h. The pH of the reaction mixture was adjusted to 7 with saturated aqueous Na₂CO₃ and concentrated under reduced pressure. The residue was diluted with H₂O (5 mL) and extracted with CH₂Cl₂ (5 mL×3). The combined organic layers were washed with brine (10 mL), dried over Na₂SO₄, filtered, and the filtrate concentrated under reduced pressure. The crude product was purified by column chromatography (SiO₂, CH₂Cl₂/MeOH, v/v, 50/1 to 1/1) to give crude (E)-4,5-difluoro-3-(2-nitrovinyl)-1H-indole (368 mg) as a yellow solid which was used in the subsequent step without further purification.

### Step 2: 2-(4,5-difluoro-1H-indol-3-yl)ethan-1-amine (281)

To a solution of crude (E)-4,5-difluoro-3-(2-nitrovinyl)-1H-indole (368 mg) in THF (0.70 mL) at 0° C. was added LiAlH₄ (685 mg, 18.0 mmol) in portions, and the reaction mixture was stirred at 25° C. for 2 h. The reaction mixture was quenched with Na₂SO₄·10H₂O (1.50 g), filtered, and the filter cake was washed with THF (0.50 mL×3). The combined filtrate was concentrated under pressure to give 2-(4,5-difluoro-1H-indol-3-yl)ethan-1-amine (288 mg) as a yellow solid which was used in the subsequent step without further purification. LCMS (ESI+): m/z 197.1 [M+H]⁺.

### Step 3: 2-(4,5-difluoro-1H-indol-3-yl)-N,N-dimethylethan-1-amine (P-142)

To an ice-cold (0° C.) solution of 2-(4,5-difluoro-1H-indol-3-yl)ethan-1-amine (288 mg) in MeOH (1.40 mL) was added NaBH(OAc)₃ (1.56 g, 7.34 mmol), 37% w/w aqueous formaldehyde solution (0.55 mL, 7.34 mmol) and AcOH (88 mg, 1.47 mmol) and the mixture was stirred at 25° C. for 3 h. The mixture was concentrated under reduced pressure to remove MeOH, and the residue was diluted with H₂O (1 mL), extracted with EtOAc (2 mL×3), and the combined organic layers were dried over Na₂SO₄, filtered, and the filtrate concentrated under reduced pressure. The crude product was purified by preparative HPLC (column: C18-1 150×30 mm×5 μm; mobile phase: [water (TFA)-ACN]; B: 1-40%, 8 min) to give 2-(4,5-difluoro-1H-indol-3-yl)-N,N-dimethylethan-1-amine as the trifluoroacetate salt (19.04 mg, 3% over 3 steps) which was a colorless oil. ¹H NMR (400 MHZ, DMSO-d₆): δ 11.4 (br s, 1H), 9.76 (br s, 1H), 7.36 (d, J=1.6 Hz, 1H), 7.12-7.19 (m, 2H), 3.35-3.25 (m, 2H), 3.17-3.12 (m, 2H), 2.86 (s, 6H). LCMS (ESI+): m/z 225.2 [M+H]⁺. HPLC purity (220 nm): 96.6%.

### Example 104:2-(4,5-difluoro-1H-indol-3-yl)-N-ethyl-N-methylethan-1-amine (P-143)

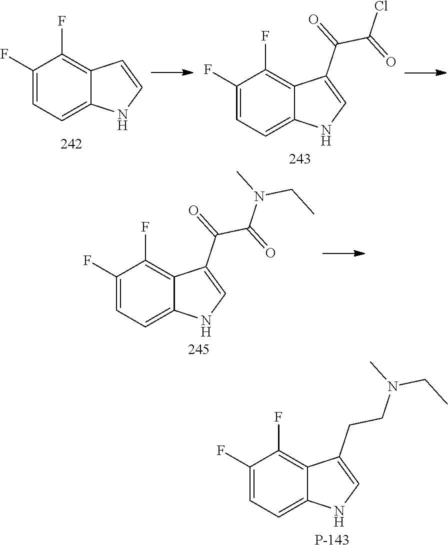

### Step 1: 2-(4,5-difluoro-1H-indol-3-yl)-2-oxoacetyl chloride (243)

A solution of oxalyl chloride (1.01 mL, 11.8 mmol) in anhydrous Et₂O (10 mL) was added dropwise to an ice-cold stirred solution of 4,5-difluoro-1H-indole (1.5 g, 9.8 mmol) in anhydrous Et₂O (20 mL). The reaction was allowed to warm to ambient temperature and stirring continued for 8 h. The resultant precipitate was filtered, washed with Et₂O (2×20 mL) to give 2-(4,5-difluoro-1H-indol-3-yl)-2-oxoacetyl chloride (1.81 g, 76%) as a yellow solid which was used in subsequent steps without further purification.

### Step 2: 2-(4,5-difluoro-1H-indol-3-yl)-N-ethyl-N-methyl-2-oxoacetamide (245)

A solution of ethyl(methyl)amine (214 mg, 3.61 mmol) in CH₂Cl₂ (5 mL) was added dropwise to an ice-cold suspension of 2-(4,5-difluoro-1H-indol-3-yl)-2-oxoacetyl chloride (0.4 g, 1.64 mmol) in CH₂Cl₂ (5 mL). Stirring was continued for 3 h at which point the reaction was diluted with H₂O (20 mL) and organic layer separated. The organic layer was washed sequentially with dilute HCl (0.1 M aq., 25 mL), H₂O (2×25 mL) followed by brine (50 mL) before being dried (MgSO₄), filtered, and the filtrate concentrated under reduced pressure. The residue was triturated with CH₂Cl₂/Et₂O (1:2 v/v, 5 mL) and filtered to give a white powder identified as the desired product 2-(4,5-difluoro-1H-indol-3-yl)-N-ethyl-N-methyl-2-oxoacetamide (410 mg, 94%) as a mixture of rotamers (A:B). ¹H NMR (400 MHZ, MeOD-d₄): δ 8.13-8.09 (m, 1H), 7.32-7.15 (m, 2H), 3.59 (q, J=7.2 Hz, 0.8H, rotamer A), 3.41 (q, J=7.2 Hz, 1.2H, rotamer B), 3.08 (s, 1.8H, rotamer B), 3.04 (s, 1.2H, rotamer A), 1.27 (t, J=7.2 Hz, 1.2H, rotamer A), 1.21 (t, J=7.2 Hz, 1.8H, rotamer B).

### Step 3: 2-(4,5-difluoro-1H-indol-3-yl)-N-ethyl-N-methylethan-1-amine (P-143)

To an ice-cold stirred solution of 2-(4,5-difluoro-1H-indol-3-yl)-N-ethyl-N-methyl-2-oxoacetamide (430 mg, 1.62 mmol) in anhydrous THF (15 mL) was added LiAlH₄ (184 mg, 3 eq., 4.85 mmol) in portions. The resulting suspension was then heated at reflux for 8 h. The suspension was then cooled in an ice bath and quenched by sequential addition of H₂O (0.15 mL), 30% NaOH (w/v) (0.15 mL), H₂O (0.9 mL). The suspension was stirred at 0° C. for a further 1 h, Na₂SO₄ was added, and the reaction mass filtered through a pad of celite. The filter cake was washed sequentially with THF (2×20 mL) and EtOAc (20 mL), and the combined filtrate was concentrated under a stream of N₂ gas to give 2-(4,5-difluoro-1H-indol-3-yl)-N-ethyl-N-methylethan-1-amine (351 mg) as a colourless oil which was used in the subsequent step without further purification.

### Step 3a: 2-(4,5-difluoro-1H-indol-3-yl)-N-ethyl-N-methylethan-1-amine oxalate (P-143⋅oxalate)

A solution of 2-(4,5-difluoro-1H-indol-3-yl)-N-ethyl-N-methylethan-1-amine (350 mg) in hot acetone (5 mL) was added dropwise to a saturated solution of oxalic acid (132 mg, 1.47 mmol) in acetone at reflux. The solution was allowed to cool to ambient temperature and the resultant yellow needles were collected by vacuum filtration and dried in a vacuum desiccator overnight yielding 2-(4,5-difluoro-1H-indol-3-yl)-N-ethyl-N-methylethan-1-amine (360 mg, 67% over 2 steps) as the oxalate salt. ¹H NMR (400 MHz, DMSO-d₆): δ 11.41 (s, 1H), 7.37-7.33 (m, 1H), 7.21-7.04 (m, 2H), 3.30-3.20 (m, 2H), 3.19-3.06 (m, 4H), 2.78 (s, 3H), 1.22 (t, J=7.2 Hz, 3H). qNMR Purity (ERETIC): 93.3%.

### Example 105: N-(2-(4,5-difluoro-1H-indol-3-yl)ethyl)-N-methylpropan-2-amine (P-144)

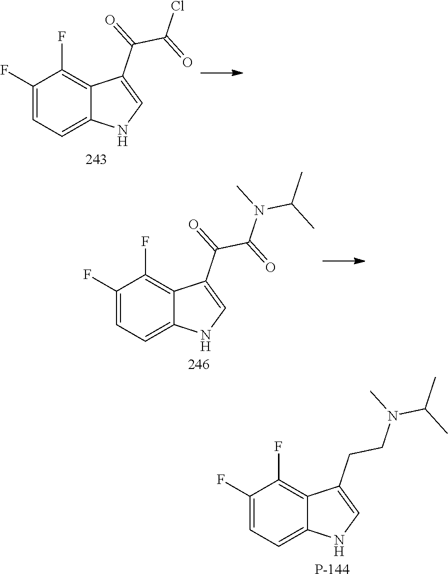

### Step 1: 2-(4,5-difluoro-1H-indol-3-yl)-N-isopropyl-N-methyl-2-oxoacetamide (246)

A solution of methyl(propan-2-yl)amine (264 mg, 2.2 eq., 3.61 mmol) in CH₂Cl₂ (5 mL) was added dropwise to an ice-cold suspension of 2-(4,5-difluoro-1H-indol-3-yl)-2-oxoacetyl chloride (0.4 g, 1.64 mmol) in CH₂Cl₂ (5 mL). Stirring was continued for 3 h at which point the reaction was diluted with H₂O (20 mL) and organic layer separated. The organic layer was washed subsequently with dilute HCl (0.1 M aq, 25 mL), H₂O (2×25 mL) followed by brine (50 mL) before being dried (MgSO₄), filtered, and concentrated under reduced pressure. The residue was triturated with CH₂Cl₂/Et₂O (1:10 v/v., 0.5 mL) and filtered to give a white powder identified as the desired product 2-(4,5-difluoro-1H-indol-3-yl)-N-isopropyl-N-methyl-2-oxoacetamide (421 mg, 91%). ¹H NMR (400 MHZ, MeOD-d₄): δ 8.35-8.05 (m, 1H), 7.59-6.96 (m, 3H), 4.03 (p, J=6.6 Hz, 1H), 3.09-2.85 (m, 3H), 1.31-1.21 (m, 6H) [mixture of rotamers].

### Step 2: N-(2-(4,5-difluoro-1H-indol-3-yl)ethyl)-N-methylpropan-2-amine (P-144)

To an ice-cold stirred solution of 2-(4,5-difluoro-1H-indol-3-yl)-N-isopropyl-N-methyl-2-oxoacetamide (490 mg, 1.75 mmol) in anhydrous THF (15 mL) was added LiAlH₄ (199 mg, 3 eq., 5.24 mmol) in portions. The resulting suspension was then heated at reflux for 8 h. The suspension was then cooled in an ice bath and quenched by subsequent addition of H₂O (0.2 mL), 30% NaOH (w/v) (0.2 mL), H₂O (1 mL). The suspension was stirred at 0° C. for a further 1 h, Na₂SO₄ was added, and the reaction mass filtered through a pad of celite. The residue was washed sequentially with THF (2×20 mL) and EtOAc (20 mL), and the filtrate was concentrated under a stream of N₂ to give 2-(4,5-difluoro-1H-indol-3-yl)-N-ethyl-N-methylethan-1-amine (350 mg, 79%) as a colourless oil. ¹H NMR (400 MHZ, MeOD-d₄): δ 7.12-7.02 (m, 2H), 6.99-6.87 (m, 1H), 3.05-2.90 (m, 3H), 2.80-2.70 (m, 2H), 2.35 (s, 3H), 1.08 (d, J=6.8 Hz, 6H).

### Example 106:2-(4,5-difluoro-1H-indol-3-yl)-N,N-diethylethan-1-amine (P-145)

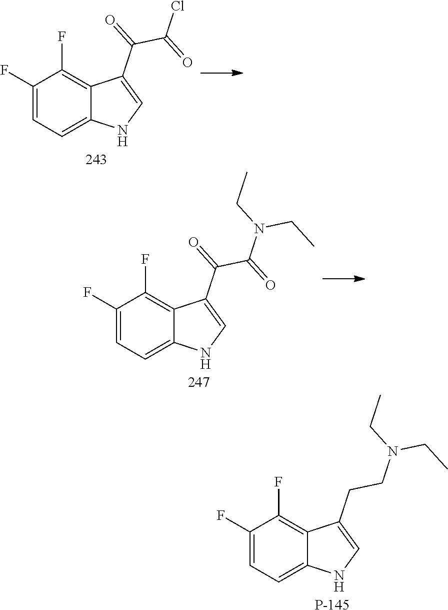

### Step 1: 2-(4,5-difluoro-1H-indol-3-yl)-N,N-diethyl-2-oxoacetamide (247)

A solution of diethylamine (174 mg, 1.5 eq., 2.38 mmol) in CH₂Cl₂ (5 mL) was added dropwise to an ice-cold suspension of 2-(4,5-difluoro-1H-indol-3-yl)-2-oxoacetyl chloride (387 mg, 1.59 mmol) in CH₂Cl₂ (5 mL). Stirring was continued for 3 h at which point the reaction was diluted with H₂O (20 mL) and the organic layer separated. The organic layer was washed sequentially with dilute HCl (0.1 M aq., 25 mL), H₂O (2×25 mL) followed by brine (50 mL) before being dried (MgSO₄), filtered, and the filtrate concentrated under reduced pressure. The residue was triturated with CH₂Cl₂/Et₂O (1:2 v/v, 5 mL) and filtered to give a white powder identified as the desired product 2-(4,5-difluoro-1H-indol-3-yl)-N,N-diethyl-2-oxoacetamide (383 mg, 86%). ¹H NMR (400 MHz, MeOD-d₄): δ 8.07 (s, 1H), 7.34-7.15 (m, 2H), 3.55 (q, J=7.2 Hz, 2H), 3.39 (q, J=7.2 Hz, 2H), 1.28 (t, J=7.2 Hz, 3H), 1.19 (t, J=7.2 Hz, 3H).

### Step 2: 2-(4,5-difluoro-1H-indol-3-yl)-N,N-diethylethan-1-amine (P-145)

To an ice-cold stirred solution of 2-(4,5-difluoro-1H-indol-3-yl)-N,N-diethyl-2-oxoacetamide (237 mg, 0.85 mmol) in anhydrous THF (15 mL) was added LiAlH₄ (96.3 mg, 3 eq., 2.54 mmol) in one portion. The resulting suspension was then heated at reflux for 8 h. The suspension was then cooled in an ice bath and quenched by sequential addition of H₂O (0.1 mL), 30% NaOH (w/v) (0.1 mL), H₂O (0.3 mL). The suspension was stirred at 0° C. for a further 1 h, Na₂SO₄ was added, and the reaction mass filtered through a pad of celite. The residue was washed sequentially with THF (2×20 mL) and EtOAc (20 mL), and the combined filtrate was concentrated under a stream of N₂ gas to give 2-(4,5-difluoro-1H-indol-3-yl)-N,N-diethylethan-1-amine (200 mg) as a colourless oil which was used in the subsequent step without further purification.

### Step 2a: 2-(4,5-difluoro-1H-indol-3-yl)-N,N-diethylethan-1-amine fumarate (P-145⋅fumarate)

A solution of 2-(4,5-difluoro-1H-indol-3-yl)-N,N-diethylethan-1-amine (200 mg) in hot acetone (2 mL) was added dropwise to a saturated solution of fumaric acid (92 mg, 0.79 mmol) in acetone at reflux. The colorless solution was allowed to cool to room temperature and held at this temperature overnight. The resultant colourless needles were collected by vacuum filtration and dried in a vacuum desiccator overnight yielding 2-(4,5-difluoro-1H-indol-3-yl)-N,N-diethylethan-1-amine (200 mg, 76% over 2 steps) as the fumarate salt. ¹H NMR (400 MHZ, DMSO-d₆): δ 11.25 (s, 1H), 7.28 (d, J=2.0 Hz, 1H), 7.13 (dd, J=8.8, 3.6 Hz, 1H), 7.10-7.00 (m, 1H), 6.49 (s, 1H), 3.00-2.80 (m, 4H), 2.75 (q, J=7.2 Hz, 4H), 1.06 (t, J=7.2 Hz, 6H). LCMS (ESI+): m/z 253.2 [M+H]⁺. qNMR Purity (ERETIC): 97.0%.

### Example 107: N-(2-(4,5-difluoro-1H-indol-3-yl)ethyl)-N-isopropylpropan-2-amine (P-146)

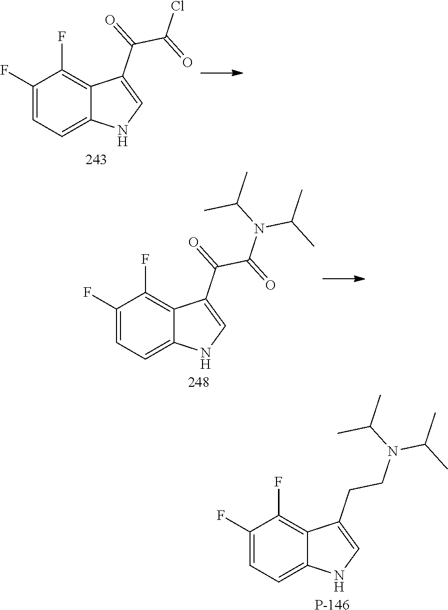

### Step 1: 2-(4,5-difluoro-1H-indol-3-yl)-N,N-diisopropyl-2-oxoacetamide (248)

A solution of N,N-diisopropylamine (366 mg, 3.61 mmol) in CH₂Cl₂ (5 mL) was added dropwise to an ice-cold suspension of 2-(4,5-difluoro-1H-indol-3-yl)-2-oxoacetyl chloride (400 mg, 1.64 mmol). Stirring was continued for 3 h at which point the reaction was diluted with H₂O (20 mL) and the organic layer separated. The organic layer was washed sequentially with dilute HCl (0.1 M aq., 25 mL), H₂O (2×25 mL) followed by brine (50 mL) before being dried (MgSO₄), filtered, and the filtrate concentrated under reduced pressure. The residue was triturated with CH₂Cl₂/Et₂O (1:2 v/v, 5 mL) and filtered to give a white powder identified as the desired product 2-(4,5-difluoro-1H-indol-3-yl)-N,N-diisopropyl-2-oxoacetamide (234 mg, 46%). ¹H NMR (400 MHZ, MeOD-d₄): δ 8.05 (s, 1H), 7.35-7.09 (m, 2H), 3.94 (sept, J=6.8 Hz, 1H), 3.71 (sept, J=6.8 Hz, 1H), 1.58 (d, J=6.8 Hz, 6H), 1.24 (d, J=6.8 Hz, 6H).

### Step 2: N-(2-(4,5-difluoro-1H-indol-3-yl)ethyl)-N-isopropylpropan-2-amine (P-146)

To an ice-cold stirred solution of 2-(4,5-difluoro-1H-indol-3-yl)-N,N-diisopropyl-2-oxoacetamide (220 mg, 0.71 mmol) in anhydrous THF (15 mL) was added LiAlH₄ (81 mg, 3 eq., 2.14 mmol) in portions. The resulting suspension was then heated at reflux for 8 h. The suspension was then cooled in an ice bath and quenched by sequential addition of H₂O (0.1 mL), 30% NaOH (w/v) (0.1 mL), H₂O (0.3 mL). The suspension was stirred at 0° C. for a further 1 h, Na₂SO₄ was added, and the reaction mass filtered through a pad of celite. The residue was washed sequentially with THF (2×20 mL) and EtOAc (20 mL), and the filtrate was concentrated under a stream of N₂ gas to give N-(2-(4,5-difluoro-1H-indol-3-yl)ethyl)-N-isopropylpropan-2-amine (140 mg) as a colourless oil which was used in the subsequent step without further purification.

### Step 2a: N-(2-(4,5-difluoro-1H-indol-3-yl)ethyl)-N-isopropylpropan-2-amine oxalate (P-146⋅oxalate)

A solution of N-(2-(4,5-difluoro-1H-indol-3-yl)ethyl)-N-isopropylpropan-2-amine (181 mg) in hot acetone (2 mL) was added dropwise to a solution of oxalic acid (60 mg, 0.66 mmol) in acetone (10 mL) at reflux. The colorless solution was allowed to cool to ambient temperature and the resultant yellow crystals were collected by vacuum filtration and dried in a vacuum desiccator overnight yielding N-(2-(4,5-difluoro-1H-indol-3-yl)ethyl)-N-isopropylpropan-2-amine (75 mg, 28% over 2 steps) as the oxalate salt. ¹H NMR (400 MHZ, DMSO-d₆): δ 11.45 (s, 1H), 7.43 (d, J=2.0 Hz, 1H), 7.25-6.97 (m, 2H), 3.77-3.53 (m, 2H), 3.34-3.01 (m, 4H), 1.31 (d, J=6.4 Hz, 12H). qNMR Purity (ERETIC): 96.8%.

Scheme 24: Compounds of general formula (I) can be synthesised from the appropriately substituted indole following the outlined sequence of steps in Scheme 24 or similar as one skilled in the art may consider. Glyoxylation of substituted indoles with oxalyl chloride followed by treatment with an appropriately substituted amine gave glyoxamide intermediates which, when subjected to reductive conditions, provided compounds of general formula (I) (exemplified by P-147). One skilled in the art will recognise that utilising differentially substituted amines would allow access to compounds of general formula (I) disclosed herein.

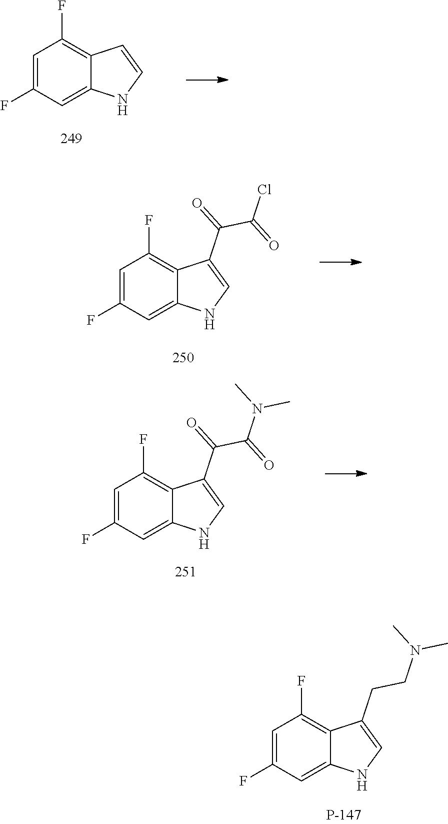

### Example 108:2-(4,6-difluoro-1H-indol-3-yl)-N,N-dimethylethan-1-amine (P-147)

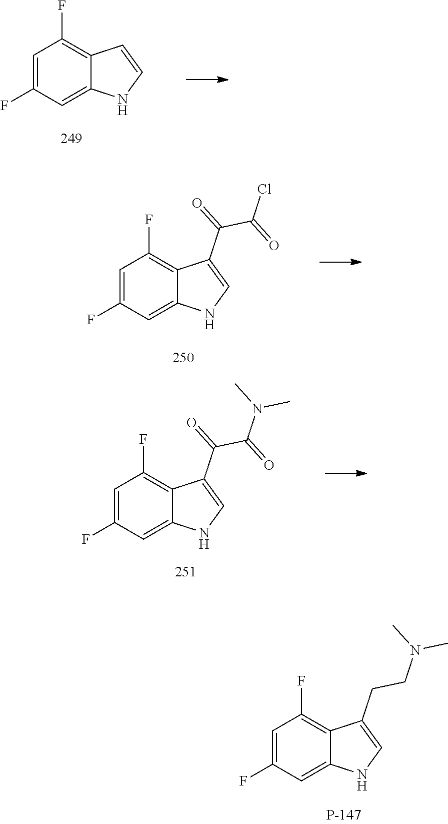 [SMILES: Fc1cc(F)c2cc[nH]c2c1 ; O=C(Cl)C(=O)c1c[nH]c2cc(F)cc(F)c12 ; CN(C)C(=O)C(=O)c1c[nH]c2cc(F)cc(F)c12 ; CN(C)CCc1c[nH]c2cc(F)cc(F)c12]

### Step 1: 2-(4,6-difluoro-1H-indol-3-yl)-2-oxoacetyl chloride (250)

To a solution of compound 4,6-difluoro-1H-indole (2.00 g, 13.1 mmol) in THF (20 mL) was added (COCl)₂ (2.49 g, 19.6 mmol) dropwise at 0° C. under N₂ atmosphere. The mixture was stirred at 0° C. for 2 h, and then concentrated under reduced pressure to give 2-(4,6-difluoro-1H-indol-3-yl)-2-oxoacetyl chloride (2.80 g) as a solid that used in the next step without further purification.

### Step 2: 2-(4,6-difluoro-1H-indol-3-yl)-N,N-dimethyl-2-oxoacetamide (251)

To a solution of 2-(4,6-difluoro-1H-indol-3-yl)-2-oxoacetyl chloride (0.90 g, 3.69 mmol) in THF (4.50 mL) was added N,N-dimethylamine (2.0 M in THF, 9.24 mL) at 0° C. The mixture was stirred at 25° C. for 12 h, and then concentrated under reduced pressure to give crude 2-(4,6-difluoro-1H-indol-3-yl)-N,N-dimethyl-2-oxoacetamide (0.60 g) as a yellow solid that was used in the next step without further purification. LCMS (ESI+): m/z 253.0 [M+H]⁺.

### Step 3: 2-(4,6-difluoro-1H-indol-3-yl)-N,N-dimethylethan-1-amine (P-147)

A solution of 2-(4,6-difluoro-1H-indol-3-yl)-N,N-dimethyl-2-oxoacetamide (0.50 g, 1.98 mmol) in THF (5 mL) was cooled to 0° C., treated with a solution of LiAlH₄ (2.50 M in THF, 9.52 mL), and then stirred at 25° C. for 3 h. The mixture was cooled to 10° C. and quenched by addition of Na₂SO₄·10H₂O (5 g). The mixture was filtered, the filter cake was washed with THF (10 mL), and the combined filtrate was concentrated. The residue was purified by preparative HPLC (column: Waters Xbridge 150×25 mm×5 μm; mobile phase: [water (NH₄HCO₃)-ACN]; B: 10-40%, 9 min) to give 2-(4,6-difluoro-1H-indol-3-yl)-N,N-dimethylethan-1-amine (45.0 mg, 5% over 2 steps) as a white solid. ¹H NMR (400 MHz, CDCl₃): δ 8.20-8.06 (m, 1H), 6.95 (d, J=2.0 Hz, 1H), 6.82 (dd, J=9.0, 2.0 Hz, 1H), 6.58 (ddd, J=10.9, 10.0, 2.0 Hz, 1H), 3.04-2.98 (m, 2H), 2.70-2.64 (m, 2H), 2.37 (s, 6H). ¹⁹F NMR (376 MHZ, CDCl₃): δ −119.0, −120.5. LCMS (ESI+): m/z 225.1 [M+H]⁺. HPLC Purity (254 nm): 98.2%.

### Example 109:2-(4,6-difluoro-1H-indol-3-yl)-N-ethyl-N-methylethan-1-amine (P-148)

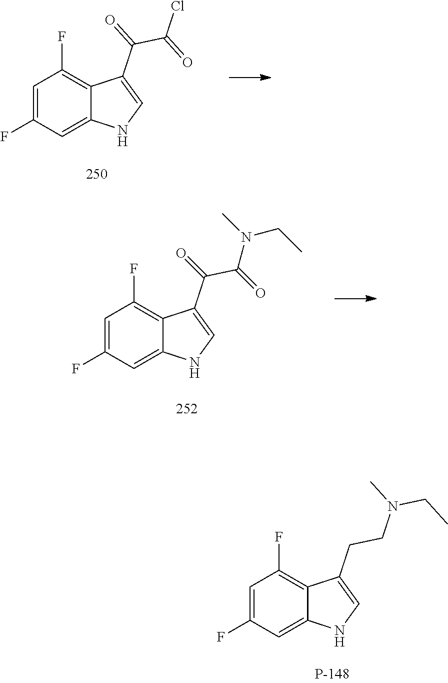 [SMILES: O=C(Cl)C(=O)c1c[nH]c2cc(F)cc(F)c12 ; CCN(C)C(=O)C(=O)c1c[nH]c2cc(F)cc(F)c12 ; CCN(C)CCc1c[nH]c2cc(F)cc(F)c12]

### Step 1: 2-(4,6-difluoro-1H-indol-3-yl)-N-ethyl-N-methyl-2-oxoacetamide (252)

To a solution of 2-(4,6-difluoro-1H-indol-3-yl)-2-oxoacetyl chloride (0.90 g, 3.69 mmol) in THF (4.50 mL) was added N-methylethanamine (1.09 g, 18.5 mmol, 1.59 mL) at 0° C. The mixture was then stirred at 25° C. for 4 h, and then concentrated under reduced pressure to give crude 2-(4,6-difluoro-1H-indol-3-yl)-N-ethyl-N-methyl-2-oxoacetamide (0.62 g) as a brown oil that was used in the next step without further purification. LCMS (ESI+): m/z 267.2 [M+H]⁺.

### Step 2: 2-(4,6-difluoro-1H-indol-3-yl)-N-ethyl-N-methylethan-1-amine (P-148)

A solution of crude 2-(4,6-difluoro-1H-indol-3-yl)-N-ethyl-N-methyl-2-oxoacetamide (0.50 g, 1.88 mmol) in THF (5 mL) was cooled to 0° C., treated with LiAlH₄ (2.50 M in THF, 9.01 mL) and stirred at 25° C. for 3 h. The mixture was cooled to 10° C. and quenched by addition of Na₂SO₄·10H₂O (5 g). The mixture was filtered, the filter cake was washed with THF (10 mL), the combined filtrate was concentrated and the resultant residue was purified by preparative HPLC (column: Waters Xbridge 150×25 mm×5 μm; mobile phase: [water (NH₄HCO₃)-ACN]; B: 10-40%, 9 min) to give 2-(4,6-difluoro-1H-indol-3-yl)-N-ethyl-N-methylethan-1-amine (28.0 mg, 6% over 2 steps) as a white solid. ¹H NMR (400 MHZ, CDCl₃): δ 8.18 (br s, 1H), 6.94 (s, 1H), 6.81 (dd, J=9.1, 1.9 Hz, 1H), 6.63-6.49 (m, 1H), 3.05-2.96 (m, 2H), 2.80-2.67 (m, 2H), 2.56 (q, J=7.2 Hz, 2H), 2.37 (s, 3H), 1.12 (t, J=7.2 Hz, 3H). ¹⁹F NMR (376 MHZ, CDCl₃): δ −119.0, −120.4. LCMS (ESI+): m/z 239.1 [M+H]⁺. HPLC Purity (254 nm): 98.4%.

### Example 110: N-(2-(4,6-difluoro-1H-indol-3-yl)ethyl)-N-methylpropan-2-amine (P-149)

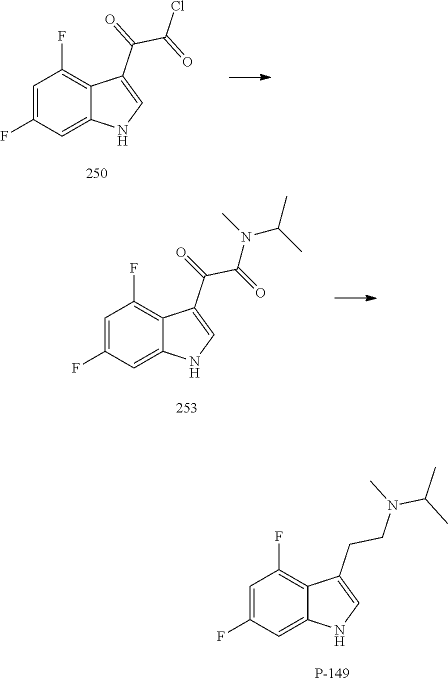 [SMILES: O=C(Cl)C(=O)c1c[nH]c2cc(F)cc(F)c12 ; CC(C)N(C)C(=O)C(=O)c1c[nH]c2cc(F)cc(F)c12 ; CC(C)N(C)CCc1c[nH]c2cc(F)cc(F)c12]

### Step 1: 2-(4,6-difluoro-1H-indol-3-yl)-N-isopropyl-N-methyl-2-oxoacetamide (253)

To a solution of 2-(4,6-difluoro-1H-indol-3-yl)-2-oxoacetyl chloride (0.90 g, 3.69 mmol) in THF (4.50 mL) was added N-methylpropan-2-amine (1.35 g, 18.5 mmol, 1.92 mL) at 0° C. The mixture was stirred at 25° C. for 4 h, concentrated under reduced pressure to give crude 2-(4,6-difluoro-1H-indol-3-yl)-N-isoproyl-N-methyl-2-oxoacetamide (0.60 g) as a brown oil that was used in the next step without further purification. LCMS (ESI+): m/z 281.2 [M+H]⁺

### Step 2: N-(2-(4,6-difluoro-1H-indol-3-yl)ethyl)-N-methylpropan-2-amine (P-149)

A solution of crude 2-(4,6-difluoro-1H-indol-3-yl)-N-isopropyl-N-methyl-2-oxoacetamide (0.50 g) in THF (5 mL) was cooled to 0° C., treated with LiAlH₄ (2.50 M in THF, 8.56 mL) and stirred at 25° C. for 3 h. The mixture was cooled to 10° C. and quenched by addition of Na₂SO₄·10H₂O (5.00 g). The mixture was filtered, the filter cake was washed with THF (10 mL), and the combined filtrate was concentrated. The residue was purified by preparative HPLC (column: Waters Xbridge 150×25 mm×5 μm; mobile phase: [water (NH₄HCO₃)-ACN]; B: 10-40%, 9 min) to give N-(2-(4,6-difluoro-1H-indol-3-yl)ethyl)-N-methylpropan-2-amine (24.0 mg, 3% over 2 steps) as a white solid. ¹H NMR (400 MHZ, CDCl₃): δ 8.13-8.04 (m, 1H), 6.97 (s, 1H), 6.84 (dd, J=9.0, 2.0 Hz, 1H), 6.65-6.47 (m, 1H), 3.07-2.95 (m, 3H), 2.84-2.63 (m, 2H), 2.37 (s, 3H), 1.08 (d, J=6.5 Hz, 6H). ¹⁹F NMR (376 MHZ, CDCl₃): δ −118.9, −120.3. LCMS (ESI+): m/z 253.1 [M+H]⁺. HPLC Purity (254 nm): 99.4%.

Scheme 25: Compounds of general formula (I) can be synthesised from the appropriately substituted indole following the outlined sequence of steps in Scheme 25 or similar as one skilled in the art may consider. Initially, a copper mediated thermal decarboxylation generated key intermediate 255 that could then undergo a similar sequence of transformations previously described. Namely, glyoxylation of substituted indoles with oxalyl chloride followed by treatment with an appropriately substituted amine gave glyoxamide intermediates which, when subjected to reductive conditions, provided compounds of general formula (I) (exemplified by P-150). One skilled in the art will recognise that utilising differentially substituted amines would allow access to compounds of general formula (I) disclosed herein.

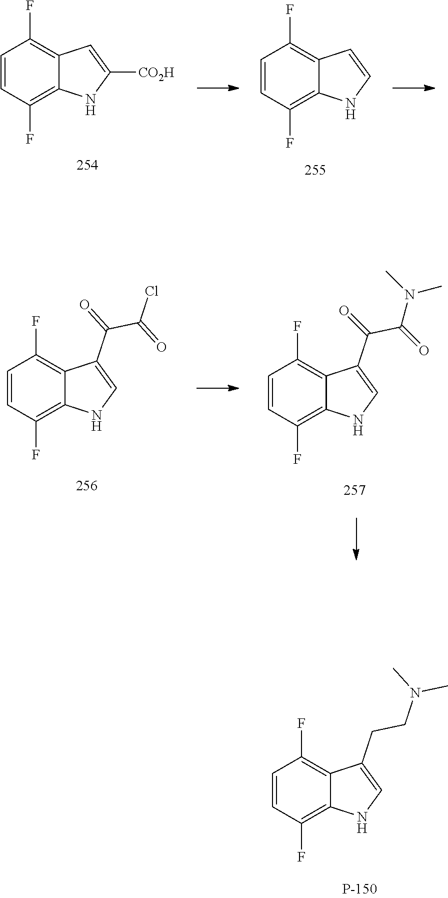

Scheme 26: Compounds of general formula (I) can be synthesised from the appropriately substituted indole following the outlined sequence of steps in Scheme 26 or similar as one skilled in the art may consider. Glyoxylation of substituted indoles with oxalyl chloride followed by treatment with an appropriately substituted amine gave glyoxamide intermediates which, when subjected to reductive conditions, provided compounds of general formula (I) (exemplified by P-153). One skilled in the art will recognise that utilising differentially substituted amines would allow access to compounds of general formula (I) disclosed herein.

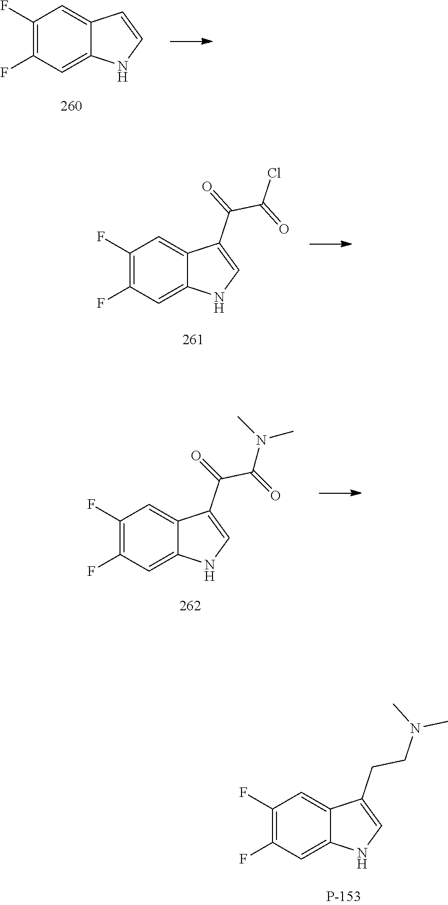

### Example 114:2-(5,6-difluoro-1H-indol-3-yl)-N,N-dimethylethan-1-amine (P-153)

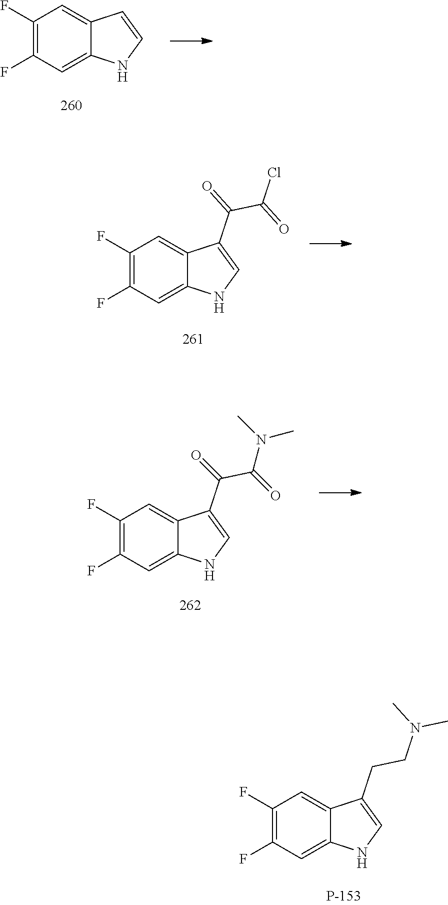 [SMILES: Fc1cc2cc[nH]c2cc1F ; O=C(Cl)C(=O)c1c[nH]c2cc(F)c(F)cc12 ; CN(C)C(=O)C(=O)c1c[nH]c2cc(F)c(F)cc12 ; CN(C)CCc1c[nH]c2cc(F)c(F)cc12]

### Step 1: 2-(5,6-difluoro-1H-indol-3-yl)-2-oxoacetyl chloride (261)

To an ice-cold solution of 5,6-difluoro-1H-indole (750 mg, 4.90 mmol) in THF (10 mL) under an atmosphere of N₂ was added (COCl)₂ (933 mg, 7.35 mmol, 643 μL) dropwise and stirring was maintained at this temperature for a further 3 h. The mixture was then concentrated under reduced pressure to give crude 2-(5,6-difluoro-1H-indol-3-yl)-2-oxoacetyl chloride (1.50 g) as a yellow solid that was used directly in the next step.

### Step 2: 2-(5,6-difluoro-1H-indol-3-yl)-N,N-dimethyl-2-oxoacetamide (262)

To a solution of 2-(5,6-difluoro-1H-indol-3-yl)-2-oxoacetyl chloride (500 mg, 2.05 mmol, 1.00 eq) in THF (5 mL) was added N,N-dimethylamine (2.00 M in THF, 5 mL) at 0° C. The mixture was stirred at 25° C. for 12 h, and then concentrated under reduced pressure to give 2-(5,6-difluoro-1H-indol-3-yl)-N,N-dimethyl-2-oxoacetamide (500 mg) as an off-white solid. ¹H NMR (400 MHZ, DMSO-d₆): δ 12.45 (br s, 1H), 8.19 (s, 1H), 7.95 (dd, J=10.8, 8.0 Hz, 1H), 7.58 (dd, J=10.8, 6.8 Hz, 1H), 2.98 (s, 3H), 2.91 (s, 3H). ¹⁹F NMR (376 MHz, DMSO-d₆): δ −141.8 (d, J=22.2 Hz), −144.2 (d, J=19.9 Hz).

### Step 3: 2-(5,6-difluoro-1H-indol-3-yl)-N,N-dimethylethan-1-amine (P-153)

A solution of 2-(5,6-difluoro-1H-indol-3-yl)-N,N-dimethyl-2-oxoacetamide (230 mg, 0.91 mmol) in THF (2.30 mL) was cooled to 0° C., treated with LiAlH₄ (2.50 M in THF, 4.38 mL, 12.0 eq) and stirred at 70° C. for 3 h under N₂ atmosphere. The mixture was cooled to 0° C. and quenched by addition of Na₂SO₄·10H₂O (1 g). The mixture was stirred at 20° C. for 10 min, filtered, and the filtrate was concentrated. The residue was purified by preparative HPLC (column: Waters Xbridge 150×25 mm×5 μm; mobile phase: [water (NH₄HCO₃)-ACN]; B: 10-40%, 9 mins) to give 2-(5,6-difluoro-1H-indol-3-yl)-N,N-dimethylethan-1-amine (45.5 mg, 10% over 2 steps) as a white solid. ¹H NMR (400 MHZ, MeOD-d₄): δ 7.35 (dd, J=11.2, 8.0 Hz, 1H), 7.19 (dd, J=11.2, 6.8 Hz, 1H), 7.13 (s, 1H), 2.94-2.88 (m, 2H), 2.69-2.63 (m, 2H), 2.37 (s, 6H). ¹⁹F NMR (376 MHZ, MeOD-d₄): δ −148.1 (d, J=20.3 Hz), −151.5 (d, J=20.3 Hz). LCMS (ESI+): m/z 225.0 [M+H]⁺. HPLC Purity (254 nm): 96.3%.

### Example 115:2-(5,6-difluoro-1H-indol-3-yl)-N-ethyl-N-methylethan-1-amine (P-154)

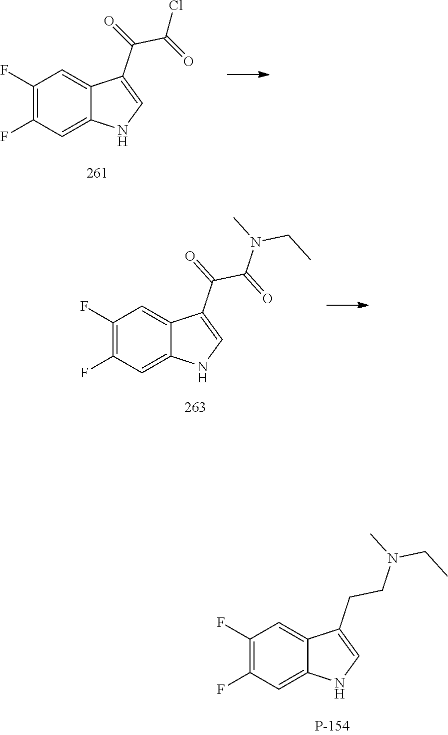 [SMILES: O=C(Cl)C(=O)c1c[nH]c2cc(F)c(F)cc12 ; CCN(C)C(=O)C(=O)c1c[nH]c2cc(F)c(F)cc12 ; CCN(C)CCc1c[nH]c2cc(F)c(F)cc12]

### Step 1: 2-(5,6-difluoro-1H-indol-3-yl)-N-ethyl-N-methyl-2-oxoacetamide (263)

To a solution of 2-(5,6-difluoro-1H-indol-3-yl)-2-oxoacetyl chloride (1.50 g, 6.16 mmol) in THF (12 mL) was added N-methylethanamine (1.82 g, 30.8 mmol, 2.65 mL) at 0° C. The mixture was stirred at 25° C. for 12 h, and concentrated under reduced pressure to give crude 2-(5,6-difluoro-1H-indol-3-yl)-N-ethyl-N-methyl-2-oxoacetamide (1.30 g) as an off-white solid which was used in the subsequent step without further purification. LCMS (ESI+): m/z 267.2 [M+H]⁺.

### Step 2: 2-(5,6-difluoro-1H-indol-3-yl)-N-ethyl-N-methylethan-1-amine (P-154)

A solution of crude 2-(5,6-difluoro-1H-indol-3-yl)-N-ethyl-N-methyl-2-oxoacetamide (300 mg) in THF (3 mL) was cooled to 0° C., treated with LiAlH₄ (2.50 M in THF, 5.41 mL, 12.0 eq) and stirred at 70° C. for 3 h under N₂ atmosphere. The mixture was cooled to 0° C. and quenched by addition of Na₂SO₄·10H₂O (1.00 g). The mixture was stirred at 20° C. for 10 min, filtered, and the filtrate was concentrated. The residue was purified by preparative HPLC (column: Waters Xbridge 150×25 mm×5 μm; mobile phase: [water (NH₄HCO₃)-ACN]; B: 10-40%, 9 mins) to give 2-(5,6-difluoro-1H-indol-3-yl)-N-ethyl-N-methylethan-1-amine (34.5 mg, 2% over 2 steps) as an off-white solid. 1H NMR (400 MHZ, MeOD-d₄): δ 7.33 (dd, J=11.2, 8.0 Hz, 1H), 7.20-7.09 (m, 2H), 2.96-2.82 (m, 2H), 2.76-2.65 (m, 2H), 2.59 (q, J=7.2 Hz, 2H), 2.36 (s, 3H), 1.13 (t, J=7.2 Hz, 3H). ¹⁹F NMR (376 MHZ, MeOD-d₄): δ −148.1 (d, J=21.1 Hz), −151.5 (d, J=21.1 Hz). LCMS (ESI+): m/z 239.2 [M+H]⁺. HPLC Purity (220 nm): 96.4%.

### Example 116: N-(2-(5,6-difluoro-1H-indol-3-yl)ethyl)-N-methylpropan-2-amine (P-155)

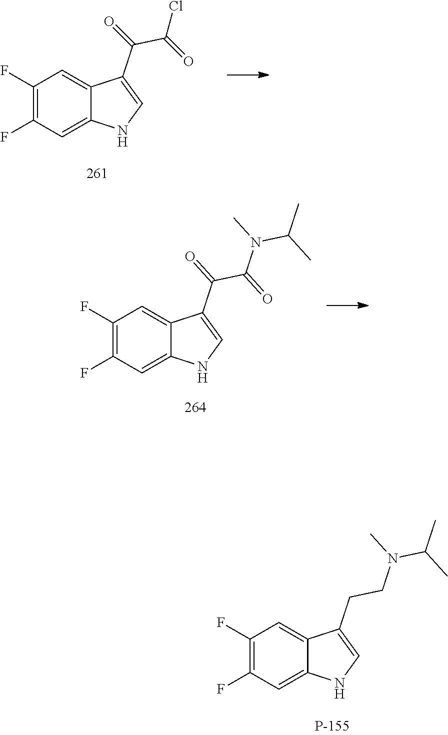 [SMILES: O=C(Cl)C(=O)c1c[nH]c2cc(F)c(F)cc12 ; CC(C)N(C)C(=O)C(=O)c1c[nH]c2cc(F)c(F)cc12 ; CC(C)N(C)CCc1c[nH]c2cc(F)c(F)cc12]

### Step 1: 2-(5,6-difluoro-1H-indol-3-yl)-N-isopropyl-N-methyl-2-oxoacetamide (264)

To a solution of 2-(5,6-difluoro-1H-indol-3-yl)-2-oxoacetyl chloride (1.50 g, 6.16 mmol) in THF (12 mL) was added N-methylpropan-2-amine (2.25 g, 30.8 mmol, 3.21 mL, 5.00 eq) at 0° C. The mixture was stirred at 25° C. for 12 h then concentrated under reduced pressure to give crude 2-(5,6-difluoro-1H-indol-3-yl)-N-isopropyl-N-methyl-2-oxoacetamide (1.70 g) as an off-white solid which was used in the subsequent step without further purification. LCMS (ESI+): m/z 281.2 [M+H]⁺.

### Step 2: N-(2-(5,6-difluoro-1H-indol-3-yl)ethyl)-N-methylpropan-2-amine (P-155)

A solution of crude 2-(5,6-difluoro-1H-indol-3-yl)-N-isopropyl-N-methyl-2-oxoacetamide (0.40 g, 1.43 mmol, 1.00 eq) in THF (3 mL) was cooled to 0° C., treated with LiAlH₄ (2.50 M solution in THF, 6.85 mL, 12.0 eq) and stirred at 70° C. for 3 h under N₂ atmosphere. The mixture was cooled to 0° C. and quenched by addition of Na₂SO₄·10H₂O (1.20 g). The mixture was stirred at 20° C. for 10 min, filtered, and the filtrate was concentrated under reduced pressure. The residue was purified by preparative HPLC (column: Waters Xbridge 150×25 mm×5 μm; mobile phase: [water (NH₄HCO₃)-ACN]; B: 10-40%, 9 mins) to give N-(2-(5,6-difluoro-1H-indol-3-yl)ethyl)-N-methylpropan-2-amine (56.6 mg, 4% over 2 steps) as an off-white solid. ¹H NMR (400 MHZ, MeOD-d₄): δ 7.34 (dd, J=11.2, 8.0 Hz, 1H), 7.19 (dd, J=11.2, 6.8 Hz, 1H), 7.13 (s, 1H), 3.01 (sept, J=6.8, 1H), 2.93-2.86 (m, 2H), 2.79-2.72 (m, 2H), 2.38 (s, 3H), 1.11 (d, J=6.8 Hz, 6H). ¹⁹F NMR (376 MHZ, MeOD-d₄): δ −148.1 (d, J=19.9 Hz), −151.6 (d, J=19.9 Hz). LCMS (ESI+): m/z 253.2 [M+H]⁺. HPLC Purity (220 nm): 97.8%.

Scheme 27: Compounds of general formula (I) can be synthesised from the appropriately substituted aniline following the outlined sequence of steps in Scheme 27 or similar as one skilled in the art may consider. Initial ortho-directed electrophilic iodination generates precursor 266 appropriately decorated for a Songashira coupling effectively producing intermediate 267. Heteroaromatic cyclisation mediated by copper complexation generated key indole intermediate 268 that could undergo a sequence of synthetic transformation previously described. Namely, glyoxylation of substituted indoles with oxalyl chloride followed by treatment with an appropriately substituted amine gave glyoxamide intermediates which, when subjected to reductive conditions, provided compounds of general formula (I) (exemplified by P-156). One skilled in the art will recognise that utilising differentially substituted amines would allow access to compounds of general formula (I) disclosed herein.

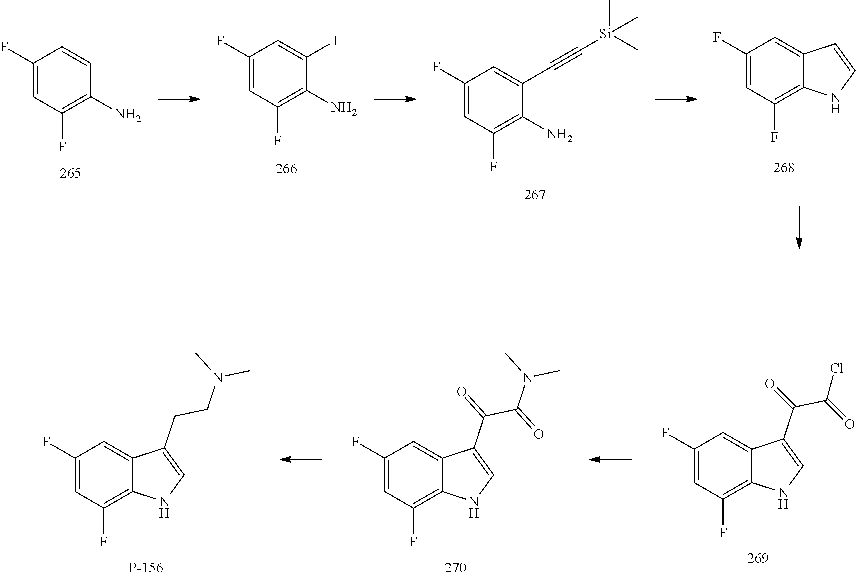

### Example 136: 2-(5,7-difluoro-1H-indol-3-yl)-N,N-dimethylethan-1-amine (P-156)

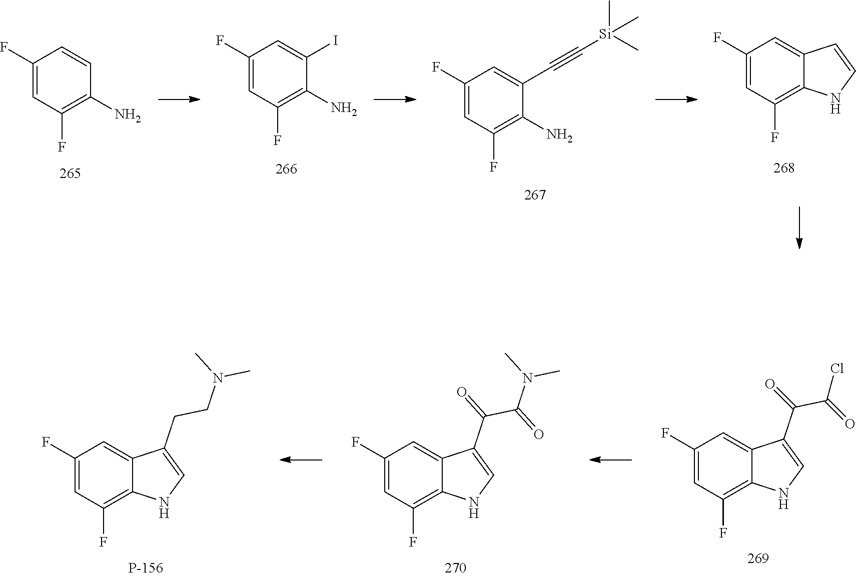

### Step 1: 2,4-difluoro-6-iodoaniline (266)

A degassed mixture of 2,4-difluoroaniline (30.0 g, 232 mmol) and NIS (52.3 g, 232 mmol) in AcOH (150 mL), purged with N₂ was stirred at 25° C. for 2 h. The reaction mixture was cooled to 0° C., quenched by the addition of H₂O (900 mL), and then extracted with EtOAc (200 mL×3). The combined organic layers were washed with brine (150 mL), dried over Na₂SO₄, filtered, and the filtrate concentrated under reduced pressure to afford crude 2,4-difluoro-6-iodoaniline (50.0 g) as a brown oil which was used in the subsequent step without further purification.

### Step 2: 2,4-difluoro-6-((trimethylsilyl)ethynyl)aniline (267)

A mixture of crude 2,4-difluoro-6-iodoaniline (49.0 g), ethynyltrimethylsilane (56.6 g, 576 mmol, 79.8 mL), Et&N (38.9 g, 384 mmol, 53.5 mL), CuI (365.9 mg, 1.92 mmol), and Pd(PPh₃)₂Cl₂ (1.35 g, 1.92 mmol) in toluene (490 mL) was degassed and purged with N₂ 3 times, before being heated to 40° C. and stirred at this temperature under atmosphere N₂ for 12 h. The reaction mixture was then cooled to 0° C., quenched by the addition of H₂O (600 mL), and extracted with EtOAc (200 mL×3). The combined organic layers were washed with brine (400 mL), dried over Na₂SO₄, filtered, and concentrated under reduced pressure to give crude 2,4-difluoro-6-((trimethylsilyl)ethynyl)aniline (38.0 g) as a brown oil which was used in the subsequent step without further purification.

### Step 3: 5,7-difluoro-1H-indole (268)

A mixture of crude 2,4-difluoro-6-((trimethylsilyl)ethynyl)aniline (20.0 g) and CuI (1.69 g, 8.88 mmol) in DMF (140 mL) was degassed and purged with N₂ 3 times, and then stirred at 100° C. for 12 h under N₂ atmosphere. The reaction mixture was then cooled to 0° C., quenched by the addition of H₂O (700 mL), and extracted with EtOAc (200 mL×3). The combined organic layers were washed with brine (200 mL), dried over Na₂SO₄, filtered, and the filtrate concentrated under reduced pressure. The crude product was purified by column chromatography (SiO₂, petroleum ether/EtOAc, v/v, 60/1 to 5/1) to give 5,7-difluoro-1H-indole (3.50 g, 26% yield) as a brown oil.

### Step 4: 2-(5,7-difluoro-1H-indol-3-yl)-2-oxoacetyl chloride (269)

To an ice-cold solution of 5,7-difluoro-1H-indole (1.00 g, 6.53 mmol) in THF (10 mL) under an atmosphere of N₂ was added (COCl)₂ (1.24 g, 9.80 mmol, 857 μL) dropwise and the mixture stirred at this temperature for a further 2 h. The mixture was then concentrated under reduced pressure to give 2-(5,7-difluoro-1H-indol-3-yl)-2-oxoacetyl chloride (1.35 g) as a brown solid that was used in the next step without further purification.

### Step 5: 2-(5,7-difluoro-1H-indol-3-yl)-N,N-dimethyl-2-oxoacetamide (270)

To a solution of crude 2-(5,7-difluoro-1H-indol-3-yl)-2-oxoacetyl chloride (0.45 g) in THF (4.50 mL) at 0° C. under N₂ atmosphere was added a solution of N,N-dimethylamine (2.00 M solution in THF, 4.62 mL) before being warmed to 25° C. and stirring maintained for a further 12 h. The mixture was concentrated under reduced pressure to give crude 2-(5,7-difluoro-1H-indol-3-yl)-N,N-dimethyl-2-oxoacetamide (0.67 g) as a brown solid that was used in the next step without further purification. LCMS (ESI+): m/z 253.2 [M+H]⁺.

### Step 3: 2-(5,7-difluoro-1H-indol-3-yl)-N,N-dimethylethan-1-amine (P-156)

To an ice-cold solution of crude 2-(5,7-difluoro-1H-indol-3-yl)-N,N-dimethyl-2-oxoacetamide (0.67 g) in THF (6 mL) under an inert atmosphere was added LiAlH₄ as a solution in THF (2.5 M, 12.8 mL) before being warmed to 70° C. and stirred for a further 3 h. The mixture was then cooled to 0° C. and quenched by the addition of Na₂SO₄·10H₂O (5.00 g). The mixture was filtered, the filter cake was washed with THF (10 mL). The combined filtrate was evaporated under reduced pressure and the resultant residue purified by preparative HPLC (column: Waters Xbridge Prep OBD C18 150×40 mm×10 μm; mobile phase: [water (NH₄HCO₃)-ACN]; B: 12-42%, 15 min) to afford 2-(5,7-difluoro-1H-indol-3-yl)-N,N-dimethylethan-1-amine (24.0 mg, 2% over 3 steps) as a brown solid. ¹H NMR (400 MHZ, CDCl₃): δ 8.23 (br s, 1H), 7.14-7.00 (m, 2H), 6.80-6.66 (m, 1H), 3.07-2.83 (m, 2H), 2.76-2.55 (m, 2H), 2.37 (s, 6H). ¹⁹F NMR (376 MHZ, CDCl₃): −122.1, −132.0. LCMS (ESI+): m/z 225.2 [M+H]⁺. HPLC purity (254 nm): 97.7%.

Scheme 28: Compounds of general formula (I) can be synthesised from the appropriately substituted aniline following the outlined sequence of steps in Scheme 28 or similar as one skilled in the art may consider. Condensation with trichloroacetaldehyde provides access to indole-2,3-diones that upon reducing conditions generated key indole intermediate 275. With the appropriately substituted indole in hand, a similar sequence of synthetic transformations could be carried out as previously described. Namely, glyoxylation of substituted indoles with oxalyl chloride followed by treatment with an appropriately substituted amine gave glyoxamide intermediates which, when subjected to reductive conditions, provided compounds of general formula (I) (exemplified by P-159). One skilled in the art will recognise that utilising differentially substituted amines would allow access to compounds of general formula (I) disclosed herein.

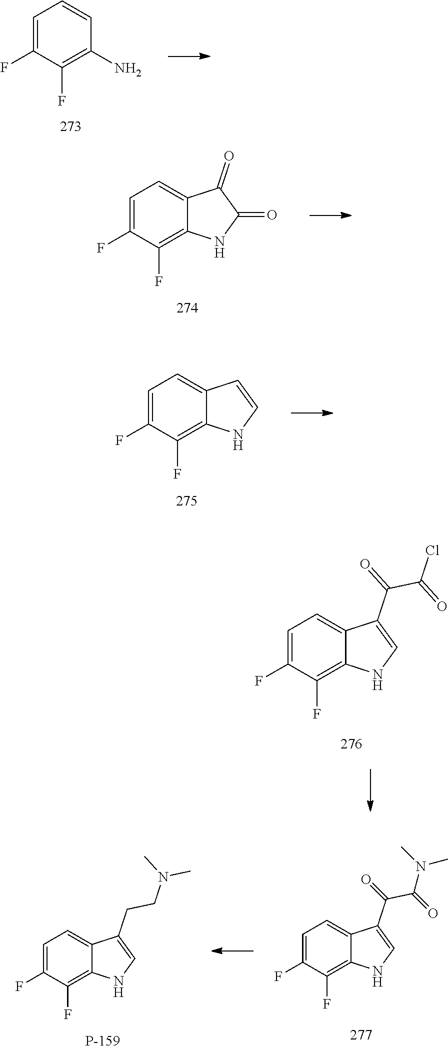

### Example 120:2-(6,7-difluoro-1H-indol-3-yl)-N,N-dimethylethan-1-amine (P-159)

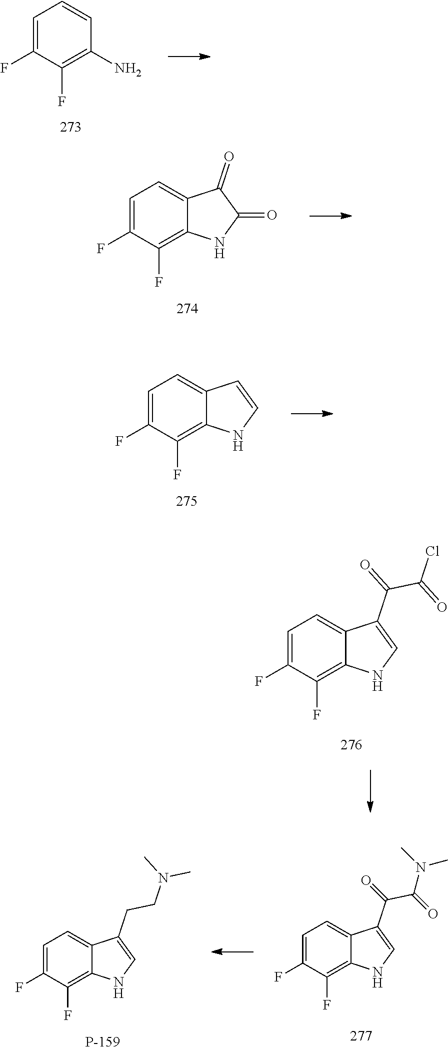 [SMILES: Nc1cccc(F)c1F ; O=C1Nc2c(ccc(F)c2F)C1=O ; Fc1ccc2cc[nH]c2c1F ; O=C(Cl)C(=O)c1c[nH]c2c(F)c(F)ccc12 ; CN(C)CCc1c[nH]c2c(F)c(F)ccc12 ; CN(C)C(=O)C(=O)c1c[nH]c2c(F)c(F)ccc12]

### Step 1: 6,7-difluoroindoline-2,3-dione (274)

A solution of 2,3-difluoroaniline (5.00 g, 38.7 mmol, 3.92 mL) in H₂O (250 mL) was treated with 2,2,2-trichloroacetaldehyde (8.56 g, 58.1 mmol), NH₂OH·HCl (9.69 g, 139 mmol) and Na₂SO₄ (44.0 g, 310 mmol) at 25° C. The mixture was stirred at 50° C. for 12 h, then cooled to 25° C., treated with 2 M aqueous HCl (20 mL) and stirred for 30 min. The formed crystals were filtered, dried, and then added to concentrated sulfuric acid (20 mL), and the mixture was stirred for 3 h at 80° C. The reaction solution was then poured onto ice-water (200 mL) and extracted with EtOAc (50 mL×2). The combined organic layers were washed with brine (50 mL), dried over Na₂SO₄, filtered, and the filtrate was concentrated under reduced pressure to give 6,7-difluoroindoline-2,3-dione (4.20 g, 59% yield) as a yellow solid. ¹H NMR (400 MHZ, DMSO-d₆): δ 11.83 (s, 1H), 7.46 (dd, J=8.4, 4.8 Hz, 1H), 7.09 (ddd, J=11.2, 8.4, 6.8 Hz, 1H). ¹⁹F NMR (376 MHz, DMSO-d₆): δ −124.5 (d, J=20.8 Hz), −157.3 (d, J=20.7 Hz).

### Step 2: 6,7-difluoro-1H-indole (275)

To an ice-cold solution of 6,7-difluoroindoline-2,3-dione (3.97 g, 21.7 mmol) in THF (40 mL) was added NaBH₄ (4.82 g, 127 mmol), then the mixture was cooled to −20° C. and treated dropwise with BF₃·Et₂O (12.3 g, 86.7 mmol, 10.7 mL). The mixture was warmed to −10° C. and stirred for 3 h, and then treated dropwise with an aqueous solution of potassium hydrogen sulfate (8.25 g in 100 mL of H₂O). The mixture was extracted with EtOAc (100 mL×3), the combined organic layers were dried (Na₂SO₄), filtered and the filtrate concentrated under reduced pressure. The residue was purified by column chromatography (SiO₂, 100% petroleum ether) to give 6,7-difluoro-1H-indole (2.02 g, 59%) as a yellow liquid. ¹H NMR (400 MHZ, CDCl₃): δ 8.38 (br s, 1H), 7.33-7.29 (m, 1H), 7.27-7.23 (m, 1H), 6.98 (dd, J=7.2, 8.4 Hz, 1H), 6.95 (dd, J=7.2, 8.8 Hz, 1H), 6.63-6.51 (m, 1H). ¹⁹F NMR (376 MHZ, CDCl₃): δ −149.2 (d, J=20.3 Hz), −161.0 (d, J=20.3 Hz).

### Step 3: 2-(6,7-difluoro-1H-indol-3-yl)-2-oxoacetyl chloride (276)

To an ice-cold solution of 6,7-difluoro-1H-indole (450 mg, 2.94 mmol) in THF (4.5 mL) was added (COCl)₂ (746 mg, 5.88 mmol) dropwise under N₂ atmosphere, and the mixture stirred at 0° C. for 7 h. The mixture was concentrated under reduced pressure to give 2-(6,7-difluoro-1H-indol-3-yl)-2-oxoacetyl chloride (600 mg) as a yellow solid which was used in the subsequent step without further purification.

### Step 4: 2-(6,7-difluoro-1H-indol-3-yl)-N,N-dimethyl-2-oxoacetamide (277)

To an ice-cold solution of crude 2-(6,7-difluoro-1H-indol-3-yl)-2-oxoacetyl chloride (700 mg) in THF (7 mL) was added dimethylamine (2 M in THF, 7.18 mL), and the reaction mixture was stirred at 25° C. for 12 h. The mixture was then filtered and the filtrate concentrated under reduced pressure to give crude 2-(6,7-difluoro-1H-indol-3-yl)-N,N-dimethyl-2-oxoacetamide (520 mg) as a yellow solid which was used in the subsequent step without further purification. LCMS (ESI+): m/z 253.1 [M+H]⁺.

### Step 5: 2-(6,7-difluoro-1H-indol-3-yl)-N,N-dimethylethan-1-amine (P-159)

To an ice-cold solution of crude 2-(6,7-difluoro-1H-indol-3-yl)-N,N-dimethyl-2-oxoacetamide (520 mg) in THF (6 mL) was added LiAlH₄ (2.5 M in THF, 9.90 mL), and then stirred at 70° C. for 3 h under an atmosphere of N₂. The mixture was then cooled to 0° C. and quenched by addition of Na₂SO₄·10H₂O (2.0 g). The mixture was stirred at 20° C. for 10 min, filtered, and the filtrate was concentrated under reduced pressure. The residue was purified by preparative HPLC (column: Waters Xbridge Prep OBD C18 150×40 mm×10 μm; mobile phase: [water (NH₄HCO₃)-ACN]; gradient: 10%-40% B over 15 mins) to give 2-(6,7-difluoro-1H-indol-3-yl)-N,N-dimethylethan-1-amine (85.0 mg, 13% over 3 steps) as an off-white solid. ¹H NMR (400 MHZ, MeOD-d₄): δ 7.26 (dd, J=8.8, 4.0 Hz, 1H), 7.12 (s, 1H), 6.89 (ddd, J=11.2, 8.8, 7.0 Hz, 1H), 3.01-2.85 (m, 2H), 2.71-2.60 (m, 2H), 2.35 (s, 6H). ¹⁹F NMR (376 MHZ, MeOD-d₄): δ −153.3 (d, J=19.4 Hz), −163.8 (d, J=19.4 Hz). LCMS (ESI+): m/z 225.2 [M+H]⁺. HPLC purity (254 nm): 97.9%.

### Example 121:2-(6,7-difluoro-1H-indol-3-yl)-N-ethyl-N-methylethan-1-amine (P-160)

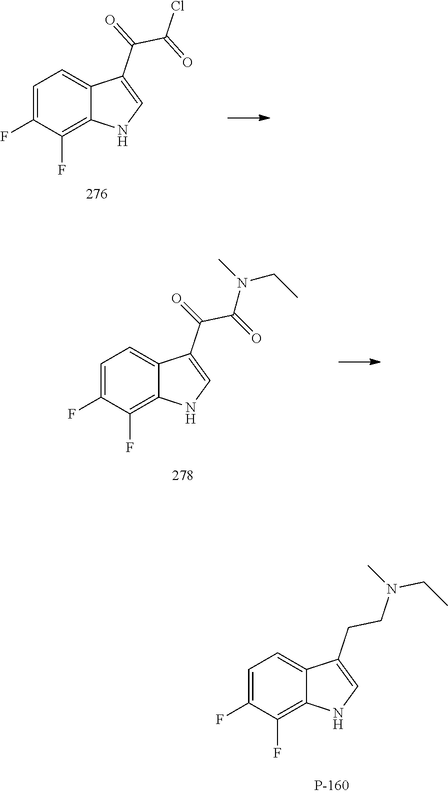 [SMILES: O=C(Cl)C(=O)c1c[nH]c2c(F)c(F)ccc12 ; CCN(C)C(=O)C(=O)c1c[nH]c2c(F)c(F)ccc12 ; CCN(C)CCc1c[nH]c2c(F)c(F)ccc12]

### Step 1: 2-(6,7-difluoro-1H-indol-3-yl)-N-ethyl-N-methyl-2-oxoacetamide (278)

To an ice-cold solution of 2-(6,7-difluoro-1H-indol-3-yl)-2-oxoacetyl chloride (600 mg, 2.46 mmol) in THF (6 mL) was added N-methylethylamine (728 mg, 12.3 mmol), and the reaction mixture was stirred at 25° C. for 12 h. The mixture was filtered, and the filtrate concentrated under reduced pressure to give crude 2-(6,7-difluoro-1H-indol-3-yl)-N-ethyl-N-methyl-2-oxoacetamide (480 mg) as a yellow solid which was used in the subsequent step without further purification.

### Step 2: 2-(6,7-difluoro-1H-indol-3-yl)-N-ethyl-N-methylethan-1-amine (P-160)

To an ice-cold solution of crude 2-(6,7-difluoro-1H-indol-3-yl)-N-ethyl-N-methyl-2-oxoacetamide (480 mg) in THF (6 mL) was added LiAlH₄ (2.5 M in THF, 8.65 mL), and then stirred at 70° C. for 3 h under an atmosphere of N₂. The mixture was then cooled to 0° C. and quenched by addition of Na₂SO₄·10H₂O (2.0 g). The mixture was stirred at 20° C. for 10 min, filtered, and the filtrate was concentrated under reduced pressure. The residue was purified by preparative HPLC (column: Waters Xbridge Prep OBD C18 150×40 mm×10 μm; mobile phase: [water (NH₄HCO₃)-ACN]; B: 10-40%, 15 mins) to give 2-(6,7-difluoro-1H-indol-3-yl)-N-ethyl-N-methylethan-1-amine (159 mg, 27% over 2 steps) as a yellow solid. ¹H NMR (400 MHZ, MeOD-d₄): δ 7.26 (dd, J=8.8, 4.0 Hz, 1H), 7.11 (s, 1H), 6.89 (ddd, J=11.2, 8.8, 7.0 Hz, 1H), 2.99-2.88 (m, 2H), 2.76-2.65 (m, 2H), 2.58 (q, J=7.2 Hz, 2H), 2.36 (s, 3H), 1.13 (t, J=7.2 Hz, 3H). ¹⁹F NMR (376 MHZ, MeOD-d₄): δ −153.27 (d, J=19.6 Hz), −163.78 (d, J=19.6 Hz). LCMS (ESI+): m/z 239.2 [M+H]⁺. HPLC purity (254 nm): 97.9%.

### Example 122: N-(2-(6,7-difluoro-1H-indol-3-yl)ethyl)-N-methylpropan-2-amine (P-161)

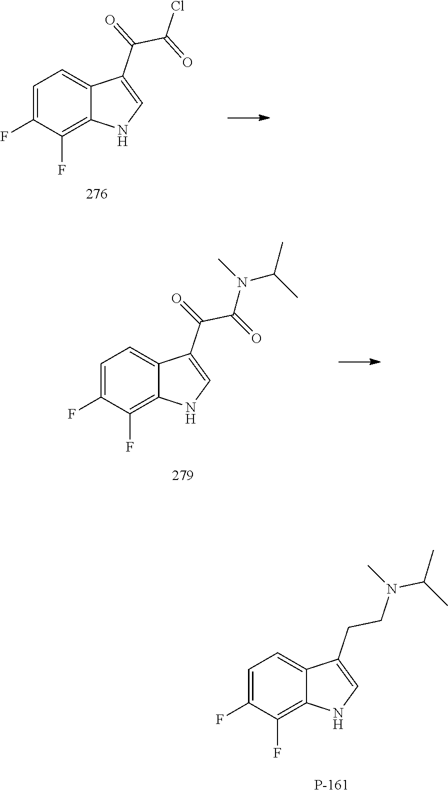 [SMILES: O=C(Cl)C(=O)c1c[nH]c2c(F)c(F)ccc12 ; CC(C)N(C)C(=O)C(=O)c1c[nH]c2c(F)c(F)ccc12 ; CC(C)N(C)CCc1c[nH]c2c(F)c(F)ccc12]

### Step 1: 2-(6,7-difluoro-1H-indol-3-yl)-N-isopropyl-N-methyl-2-oxoacetamide (279)

To an ice-cold (0° C.) solution of 2-(6,7-difluoro-1H-indol-3-yl)-2-oxoacetyl chloride (600 mg, 2.46 mmol) dissolved in THF (6 mL) was added N-methylpropan-2-amine (728 mg, 12.3 mmol), and the reaction mixture allowed to stir at 25° C. for 12 h. The mixture was then filtered, and the filtrate concentrated under reduced pressure to give crude 2-(6,7-difluoro-1H-indol-3-yl)-N-isopropyl-N-methyl-2-oxoacetamide (600 mg) as a brown solid which was used in the subsequent step without further purification. LCMS (ESI+): m/z 281.2 [M+H]⁺.

### Step 2: N-(2-(6,7-difluoro-1H-indol-3-yl)ethyl)-N-methylpropan-2-amine (P-161)

To an ice-cold (0° C.) solution of crude 2-(6,7-difluoro-1H-indol-3-yl)-N-isopropyl-N-methyl-2-oxoacetamide (600 mg) in THF (6 mL) was added LiAlH₄ (2.5 M in THF, 10.3 mL), and then stirred at 70° C. for 3 h under N₂ atmosphere. The mixture was cooled to 0° C. and quenched by addition of Na₂SO₄·10H₂O (2.00 g). The mixture was stirred at 20° C. for 10 min, filtered, and the filtrate was concentrated under reduced pressure. The residue was purified by preparative HPLC (column: Waters Xbridge Prep OBD C18 150×40 mm×10 μm; mobile phase: [water (NH₄HCO₃)-ACN]; gradient: 12%-42% B over 15 mins) to give N-(2-(6,7-difluoro-1H-indol-3-yl)ethyl)-N-methylpropan-2-amine (129 mg, 21% over 2 steps) as an off-white solid. ¹H NMR (400 MHZ, MeOD-d₄): δ 7.26 (dd, J=8.8, 4.0 Hz, 1H), 7.13 (s, 1H), 6.94-6.86 (m, 1H), 3.05-2.96 (m, 1H), 2.94-2.89 (m, 2H), 2.79-2.73 (m, 2H), 2.37 (s, 3H), 1.09 (d, J=6.8 Hz, 6H). ¹⁹F NMR (376 MHZ, MeOD-d₄): δ −153.2 (d, J=18.8 Hz), −163.7 (d, J=20.0 Hz). LCMS (ESI+): m/z 253.2 [M+H]⁺. HPLC Purity (254 nm): 97.1%.

### Example 123: Functional Assays 5-HT_{2A}, 5-HT_{2B} AND 5-HT_{2C} Receptors

Activity at 5-HT_{2A}, 5-HT_{2B} and 5-HT_{2C} receptors was determined using a FLIPR Ca²⁺ flux assay at WuXi AppTec Co. Ltd. (Hong Kong) Discovery Biology Unit according to their standard protocols. Briefly, stably transfected cells expressing the receptor of interest (HEK293 for 5-HT_{2A} and 5-HT_{2C}; CHO-K1 for 5-HT_{2B}) were grown and plated in a 384 well plate and incubated at 37° C. and 5% CO₂ overnight. A 250 mM stock solution of probenecid in FLIPR calcium assay buffer (10 mL) was freshly prepared and combined with a fluorescent dye (Fluo-4 Direct) to give a final assay concentration of 2.5 mM. Reference compounds were 4-fold serially diluted and the screening compounds were 3-fold serially diluted in 100% DMSO for 10 points using Agilent Bravo, and 750 nL was added to a 384 well compound plate using Echo along with 30 μL assay buffer. The fluorescent dye was then added to the assay plate along with assay buffer to a final volume of 40 L. The cell plate was incubated for 50 min at 37° C. and 5% CO₂ and placed into the FLIPR Tetra along with the compound plate. 10 μL of references and compounds were then transferred from the compound plate into the cell plate and the fluorescent signal was read.

TABLE 1
Agonist activity of exemplified compounds at selected serotonin (5-HT)
receptors in Ca²⁺ flux functional assays.

| | | 5-HT_{2A} | | 5-HT_{2B} | | 5-HT_{2C} | |
|---|---|---|---|---|---|---|---|
| | | EC₅₀ | Eₘₐₓ | EC₅₀ | Eₘₐₓ | EC₅₀ | Eₘₐₓ |
| Cmpd | Structure | (nM) | (%) | (nM) | (%) | (nM) | (%) |
| Example P-1 | 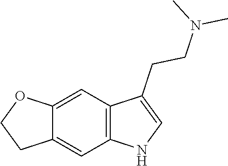 [SMILES: CN(C)CCc1c[nH]c2cc3c(cc12)OCC3] | 138.6 | 63.89 | >10000 | 2.92 | 133.7 | 82.33 |
| Example P-2 | 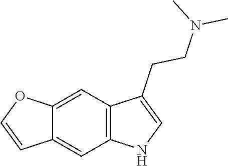 [SMILES: CN(C)CCc1c[nH]c2cc3ccoc3cc12] | 206.9 | 46.45 | >10000 | −1.07 | 56.85 | 80.56 |
| Example P-3 | 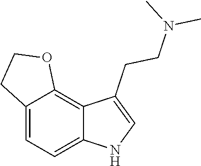 [SMILES: CN(C)CCc1c[nH]c2ccc3c(c12)OCC3] | 9.728 | 80.95 | >10000 | 13.82 | 16.22 | 76.41 |
| Example P-4 | 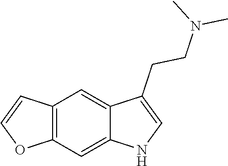 [SMILES: CN(C)CCc1c[nH]c2cc3occc3cc12] | 9.394 | 70.40 | >10000 | 4.68 | 6.114 | 78.94 |
| Example P-6 | 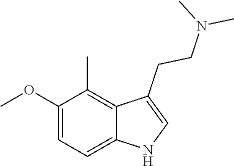 | 124.5 | 64.14 | >10000 | 11.26 | 303.1 | 73.63 |
| Example P-37 | 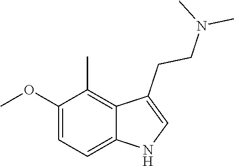 | 327.3 | 80.27 | 650.2 | 30.86 | >10000 | 53.12 |
| Example P-38 | 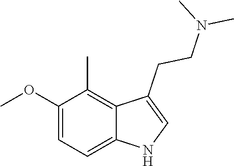 | 188.7 | 83.27 | 658.7 | 45.43 | >10000 | 23.69 |
| Example P-40 | 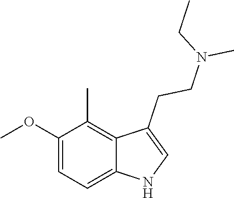 | 894.1 | 78.41 | >10000 | 33.80 | >10000 | 0.71 |
| Example P-7 | 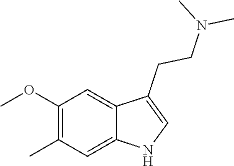 | 15.77 | 81.94 | >10000 | 1.71 | 110 | 81.06 |
| Example P-42 | 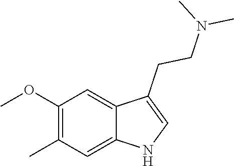 | 26.47 | 88.18 | >10000 | 8.39 | 68.46 | 85.78 |
| Example P-44 | 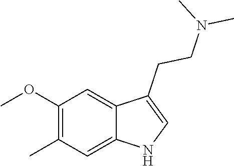 | 61.89 | 72.58 | >10000 | 7.11 | 629.7 | 70.75 |
| Example P-45 | 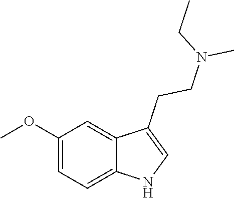 | 78.15 | 81.54 | 845.5 | 23.83 | 700.2 | 84.59 |
| Example P-46 | 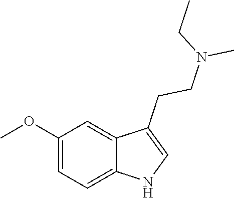 | 33.34 | 86.81 | 524.4 | 37.67 | 777.5 | 82.79 |
| Example P-47 | 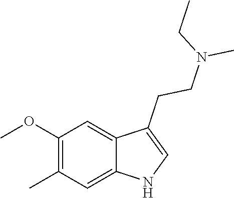 | 260.1 | 64.72 | 1074 | 43.19 | 1433 | 80.81 |
| Example P-48 | 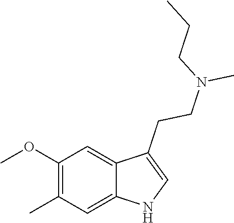 | 79.24 | 77.46 | 399 | 56.82 | 3532 | 73.99 |
| Example P-49 | 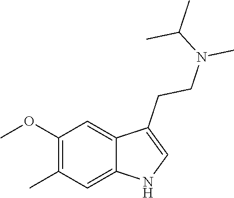 | 255.8 | 83.52 | 363.5 | 74.85 | 625.3 | 86.99 |
| Example P-50 | 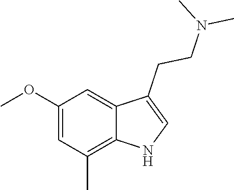 | 63.99 | 66.17 | >10000 | 0.50 | 538 | 56.40 |
| Example P-51 | 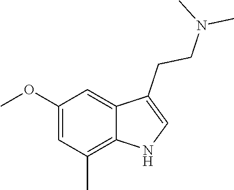 | 115.4 | 69.16 | >10000 | 0.85 | 272.5 | 57.27 |
| Example P-52 | 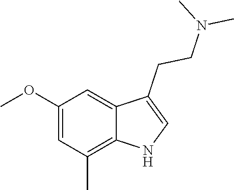 | 160.6 | 58.05 | >10000 | 1.37 | 528.7 | 44.54 |
| Example P-53 | 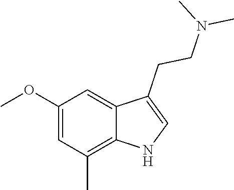 | 64.21 | 90.53 | >10000 | 1.72 | >10000 | 19.78 |
| Example P-54 | 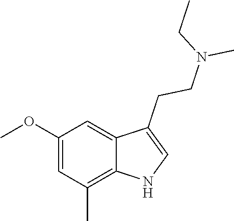 | 248.7 | 82.79 | >10000 | 7.69 | 1747 | 57.96 |
| Example P-55 | 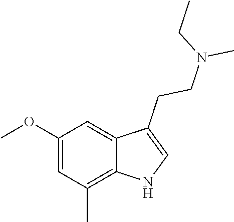 | 133.9 | 81.81 | >10000 | 13.24 | 1812 | 57.88 |
| Example P-56 | 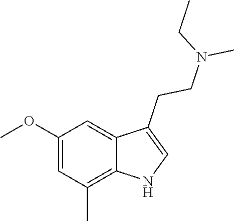 | 843.8 | 73.14 | >10000 | 5.84 | 912.6 | 82.76 |
| Example P-57 | 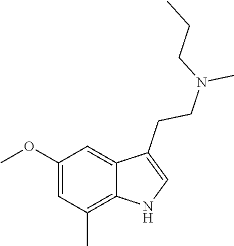 | 140.4 | 79.22 | >10000 | 9.45 | 2856 | 30.70 |
| Example P-58 | 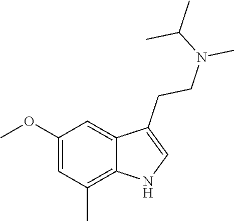 | 474.9 | 84.02 | 604 | 42.77 | 1705 | 85.77 |
| Example P-9 | 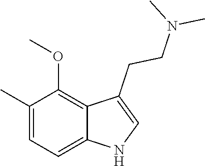 | 24.57 | 64.95 | >10000 | 17.28 | 6.231 | 80.68 |
| Example P-59 | 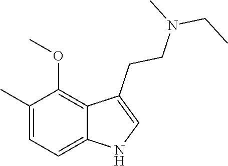 [SMILES: CCN(C)CCc1c[nH]c2ccc(C)c(OC)c12] | 34.64 | 77.99 | 4708 | 68.14 | 43.81 | 90.55 |
| Example P-60 | 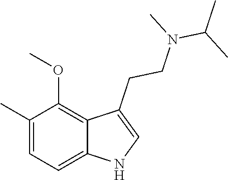 [SMILES: COc1c(C)ccc2[nH]cc(CCN(C)C(C)C)c12] | 77.64 | 66.31 | 768.4 | 70.98 | 198.4 | 72.16 |
| Example P-61 | 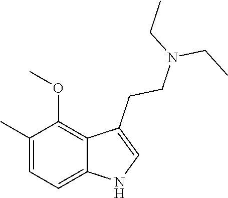 [SMILES: CCN(CC)CCc1c[nH]c2ccc(C)c(OC)c12] | 321.0 | 67.66 | >10000 | 46.31 | 443.5 | 75.76 |
| Example P-62 | 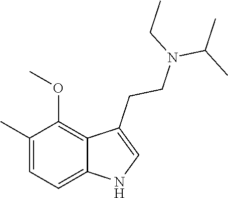 [SMILES: CCN(CCc1c[nH]c2ccc(C)c(OC)c12)C(C)C] | 318.8 | 41.87 | 5320 | 61.52 | 1290 | 65.86 |
| Example P-63 | 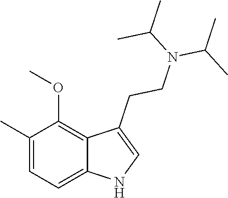 [SMILES: COc1c(C)ccc2[nH]cc(CCN(C(C)C)C(C)C)c12] | 1569 | 72.69 | 2253 | 70.20 | 9655 | 49.44 |
| Example P-10 | 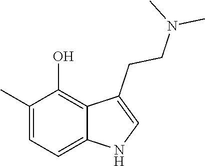 | 55.13 | 63.09 | >10000 | −0.21 | 72.83 | 71.20 |
| Example P-64 | 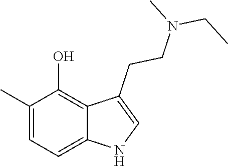 [SMILES: CCN(C)CCc1c[nH]c2ccc(C)c(O)c12] | 62.19 | 79.03 | 10000 | 2.42 | 141.1 | 60.96 |
| Example P-65 | 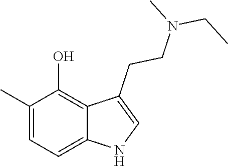 [SMILES: CCN(C)CCc1c[nH]c2ccc(C)c(O)c12] | 56.12 | 78.84 | 10000 | 6.22 | 174.9 | 66.35 |
| Example P-66 | 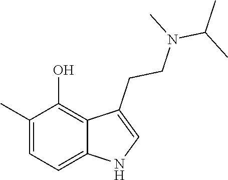 [SMILES: Cc1ccc2[nH]cc(CCN(C)C(C)C)c2c1O] | 91.89 | 94.69 | 10000 | 2.07 | 390.8 | 50.26 |
| Example P-67 | 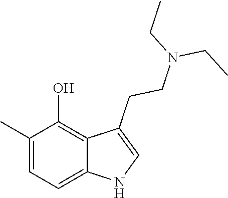 [SMILES: CCN(CC)CCc1c[nH]c2ccc(C)c(O)c12] | 162.7 | 78.33 | 10000 | 11.72 | 922.0 | 78.15 |
| Example P-68 | 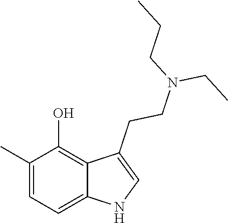 [SMILES: CCCN(CC)CCc1c[nH]c2ccc(C)c(O)c12] | 147.9 | 101.99 | 10000 | 64.27 | 871.1 | 78.01 |
| Example P-69 | 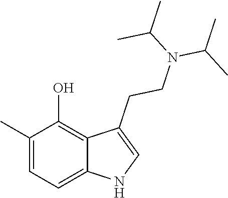 [SMILES: Cc1ccc2[nH]cc(CCN(C(C)C)C(C)C)c2c1O] | 244.9 | 78.85 | 10000 | 66.94 | 3206 | 70.24 |
| Example P-11 | 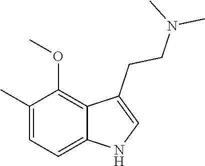 | 22.63 | 63.73 | >10000 | 0.19 | 25.68 | 74.32 |
| Example P-70 | 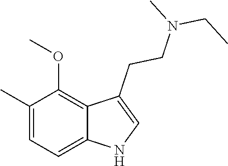 [SMILES: CCN(C)CCc1c[nH]c2ccc(C)c(OC)c12] | 71.27 | 64.68 | >10000 | 0.25 | 305.3 | 54.66 |
| Example P-71 | 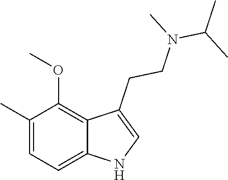 [SMILES: COc1c(C)ccc2[nH]cc(CCN(C)C(C)C)c12] | 56.68 | 70.74 | >10000 | 0.35 | 2222 | 27.92 |
| Example P-72 | 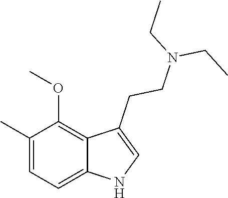 [SMILES: CCN(CC)CCc1c[nH]c2ccc(C)c(OC)c12] | 279.5 | 57.54 | >10000 | 1.13 | 1675 | 54.82 |
| Example P-73 | 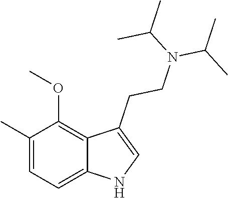 [SMILES: COc1c(C)ccc2[nH]cc(CCN(C(C)C)C(C)C)c12] | 7494 | 53.06 | 1017 | 68.08 | 6125 | 63.93 |
| Example P-12 | 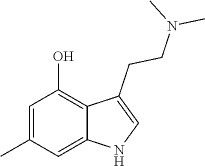 | 80.43 | 67.79 | >10000 | 1.23 | 144.4 | 69.34 |
| Example P-13 | 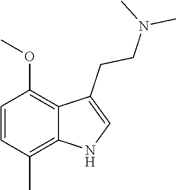 [SMILES: COc1ccc(C)c2[nH]cc(CCN(C)C)c12] | 106.8 | 36.06 | >10000 | 0.67 | 56.00 | 69.53 |
| Example P-80 | 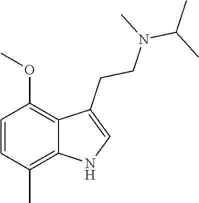 [SMILES: COc1ccc(C)c2[nH]cc(CCN(C)C(C)C)c12] | 112.7 | 29.98 | >10000 | 0.74 | 140.4 | 66.65 |
| Example P-81 | 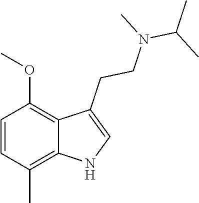 [SMILES: COc1ccc(C)c2[nH]cc(CCN(C)C(C)C)c12] | 125.9 | 41.41 | >10000 | 0.59 | 764.3 | 38.75 |
| Example P-82 | 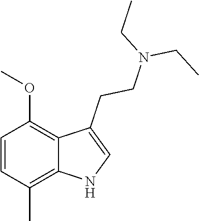 [SMILES: CCN(CC)CCc1c[nH]c2c(C)ccc(OC)c12] | 282.6 | 42.39 | >10000 | 0.89 | 416.9 | 76.67 |
| Example P-83 | 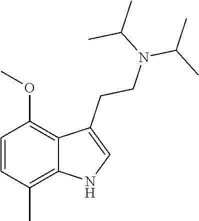 [SMILES: COc1ccc(C)c2[nH]cc(CCN(C(C)C)C(C)C)c12] | 619.3 | 79.20 | >10000 | 30.02 | 1625 | 66.74 |
| Example P-85 | 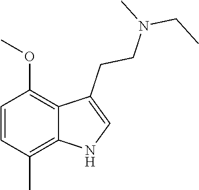 [SMILES: CCN(C)CCc1c[nH]c2c(C)ccc(OC)c12] | 62.72 | 52.01 | 10000 | 0.13 | 187.2 | 26.93 |
| Example P-86 | 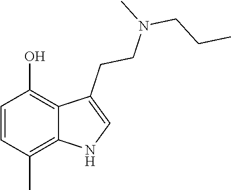 [SMILES: CCCN(C)CCc1c[nH]c2c(C)ccc(O)c12] | 109.6 | 66.86 | 10000 | −0.14 | >10000 | 10.04 |
| Example P-87 | 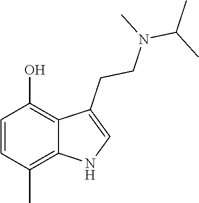 [SMILES: Cc1ccc(O)c2c(CCN(C)C(C)C)c[nH]c12] | 112.8 | 62.40 | 10000 | −0.38 | >10000 | 4.33 |
| Example P-88 | 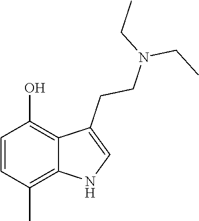 [SMILES: CCN(CC)CCc1c[nH]c2c(C)ccc(O)c12] | 188.7 | 61.13 | 10000 | −0.07 | 1296 | 25.42 |
| Example P-89 | 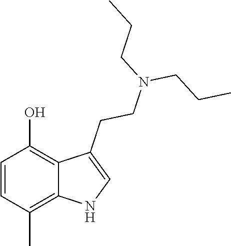 [SMILES: CCCN(CCC)CCc1c[nH]c2c(C)ccc(O)c12] | 48.62 | 118.09 | 10000 | 6.63 | 889.2 | 54.69 |
| Example P-90 | 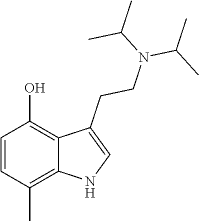 [SMILES: Cc1ccc(O)c2c(CCN(C(C)C)C(C)C)c[nH]c12] | 817.2 | 95.09 | 10000 | 15.66 | 9972 | 50.17 |
| Example P-91 | 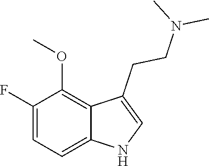 [SMILES: COc1c(F)ccc2[nH]cc(CCN(C)C)c12] | 2.328 | 74.87 | >10000 | 4.573 | 1.240 | 109.1 |
| Example P-92 | 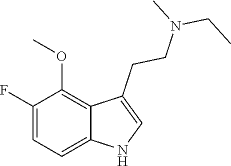 [SMILES: CCN(C)CCc1c[nH]c2ccc(F)c(OC)c12] | 7.330 | 77.98 | >10000 | 25.10 | 17.90 | 75.26 |
| Example P-93 | 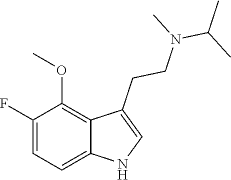 [SMILES: COc1c(F)ccc2[nH]cc(CCN(C)C(C)C)c12] | 7.252 | 95.32 | 24.76 | 51.45 | 45.16 | 85.14 |
| Example P-94 | 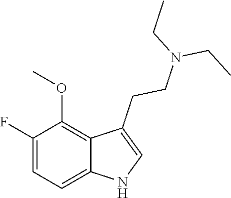 [SMILES: CCN(CC)CCc1c[nH]c2ccc(F)c(OC)c12] | 11.19 | 98.12 | 94.21 | 47.37 | 130.9 | 57.96 |
| Example P-95 | 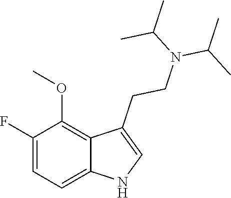 [SMILES: COc1c(F)ccc2[nH]cc(CCN(C(C)C)C(C)C)c12] | 19.99 | 93.21 | 21.55 | 96.28 | 259.7 | 83.00 |
| Example P-14 | 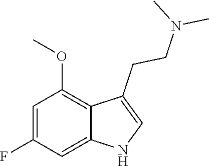 [SMILES: COc1cc(F)cc2[nH]cc(CCN(C)C)c12] | 12.93 | 87.19 | >10000 | 0.17 | 2.596 | 75.71 |
| Example P-96 | 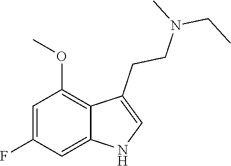 [SMILES: CCN(C)CCc1c[nH]c2cc(F)cc(OC)c12] | 7.995 | 73.64 | >10000 | 19.55 | 60.21 | 79.62 |
| Example P-97 | 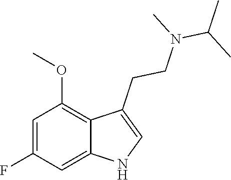 [SMILES: COc1cc(F)cc2[nH]cc(CCN(C)C(C)C)c12] | 6.764 | 65.44 | >10000 | 14.21 | 336.1 | 59.80 |
| Example P-98 | 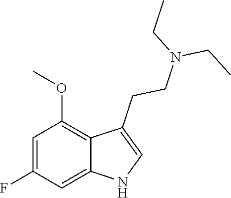 [SMILES: CCN(CC)CCc1c[nH]c2cc(F)cc(OC)c12] | 15.98 | 64.35 | 5434 | 63.58 | 310.7 | 71.82 |
| Example P-99 | 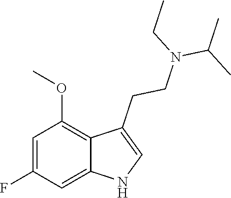 [SMILES: CCN(CCc1c[nH]c2cc(F)cc(OC)c12)C(C)C] | 30.74 | 81.44 | 912.9 | 116.47 | 754.5 | 79.29 |
| Example P-100 | 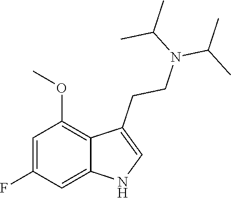 [SMILES: COc1cc(F)cc2[nH]cc(CCN(C(C)C)C(C)C)c12] | 51.43 | 91.28 | 436.0 | 127.89 | 863.9 | 76.86 |
| Example P-101 | 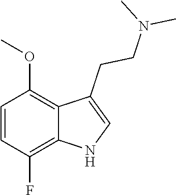 [SMILES: COc1ccc(F)c2[nH]cc(CCN(C)C)c12] | 3.842 | 71.56 | >10000 | 4.06 | 8.278 | 53.13 |
| Example P-102 | 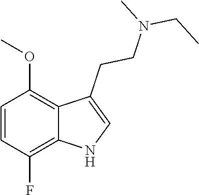 [SMILES: CCN(C)CCc1c[nH]c2c(F)ccc(OC)c12] | 14.97 | 60.81 | >10000 | 3.44 | 154.7 | 47.49 |
| Example P-103 | 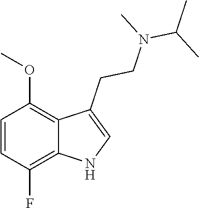 [SMILES: COc1ccc(F)c2[nH]cc(CCN(C)C(C)C)c12] | 34.56 | 61.77 | >10000 | 3.67 | >10000 | 7.02 |
| Example P-104 | 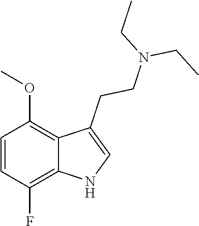 [SMILES: CCN(CC)CCc1c[nH]c2c(F)ccc(OC)c12] | 28.30 | 77.51 | >10000 | 13.08 | 8181 | 52.53 |
| Example P-105 | 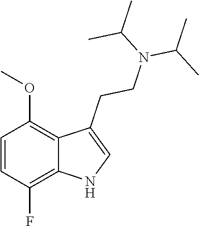 [SMILES: COc1ccc(F)c2[nH]cc(CCN(C(C)C)C(C)C)c12] | 147.1 | 91.20 | 134.2 | 126.67 | 1196 | 92.56 |
| Example P-106 | 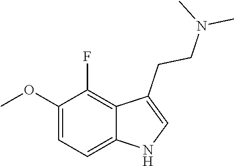 | 52.1 | 99.59 | >10000 | 5.29 | 25.39 | 93.06 |
| Example P-107 | 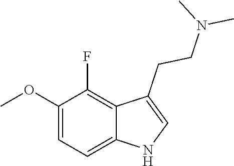 | 99.49 | 89.89 | >10000 | 15.06 | 342.7 | 87.22 |
| Example P-108 | 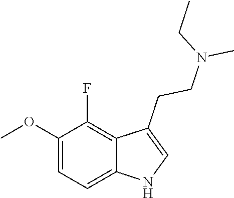 | 120.2 | 88.19 | >10000 | 33.44 | 418.2 | 86.34 |
| Example P-109 | 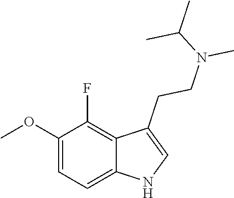 | 114.2 | 95.38 | 481.3 | 107.28 | 894.8 | 90.69 |
| Example P-115 | 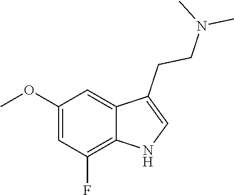 | 8.954 | 85.59 | >10000 | 16.41 | 127.4 | 81.68 |
| Example P-116 | 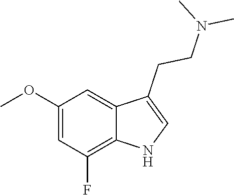 | 15.68 | 60.17 | 136.0 | 23.15 | 719.6 | 60.54 |
| Example P-117 | 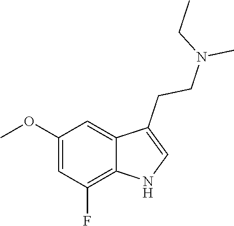 | 22.27 | 95.85 | 119.6 | 41.19 | 541.1 | 81.50 |
| Example P-118 | 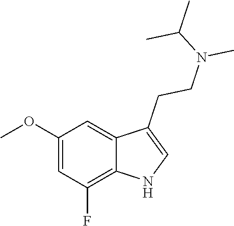 | 17.45 | 82.20 | 66.31 | 95.21 | 695.3 | 97.10 |
| Example P-125 | 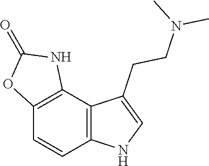 [SMILES: CN(C)CCc1c[nH]c2ccc3oc(=O)[nH]c3c12] | 48.53 | 75.46 | 14.34 | 42.55 | 538.0 | 56.68 |
| Example P-126 | 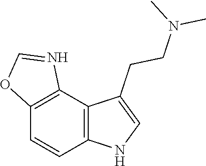 [SMILES: CN(C)CCc1c[nH]c2ccc3ocnc3c12] | 7.105 | 69.15 | >10000 | 16.72 | 5.219 | 76.72 |
| Example P-127 | 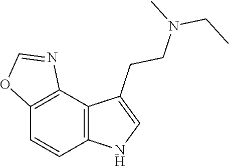 [SMILES: CCN(C)CCc1c[nH]c2ccc3ocnc3c12] | 11.96 | 54.00 | >10000 | 10.51 | 30.72 | 70.54 |
| Example P-128 | 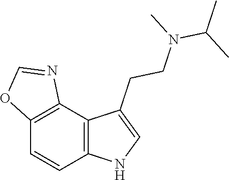 [SMILES: CC(C)N(C)CCc1c[nH]c2ccc3ocnc3c12] | 14.30 | 83.78 | >10000 | 16.78 | 67.75 | 57.99 |
| Example P-129 | 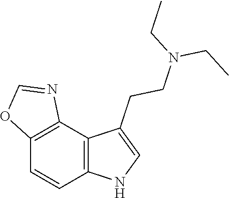 [SMILES: CCN(CC)CCc1c[nH]c2ccc3ocnc3c12] | 14.41 | 64.00 | 14.95 | 41.12 | 72.66 | 93.12 |
| Example P-130 | 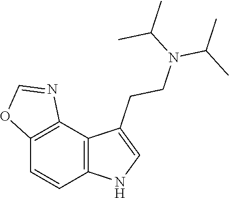 [SMILES: CC(C)N(CCc1c[nH]c2ccc3ocnc3c12)C(C)C] | 18.06 | 87.46 | 7.543 | 114.29 | 202.8 | 103.52 |
| Example P-131 | 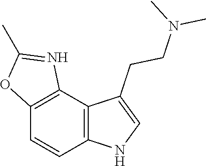 | 10.58 | 97.20 | >10000 | 3.932 | 15.78 | 90.10 |
| Example P-132 | 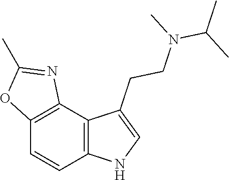 [SMILES: Cc1nc2c(ccc3[nH]cc(CCN(C)C(C)C)c32)o1] | 20.55 | 69.07 | >10000 | 11.39 | 177.4 | 85.67 |
| Example P-133 | 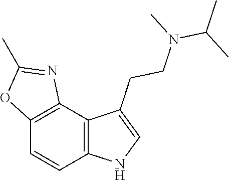 [SMILES: Cc1nc2c(ccc3[nH]cc(CCN(C)C(C)C)c32)o1] | 27.88 | 78.40 | 68.60 | 22.37 | 893.6 | 72.62 |
| Example P-134 | 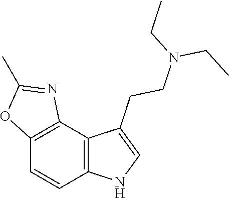 [SMILES: CCN(CC)CCc1c[nH]c2ccc3oc(C)nc3c12] | 20.27 | 77.51 | 59.91 | 56.41 | 428.7 | 91.28 |
| Example P-135 | 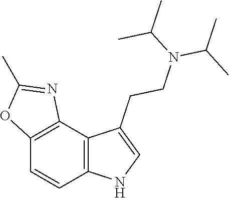 [SMILES: Cc1nc2c(ccc3[nH]cc(CCN(C(C)C)C(C)C)c32)o1] | 25.1 | 78.34 | 31.43 | 91.26 | 2066 | 99.20 |
| Example P-142 | 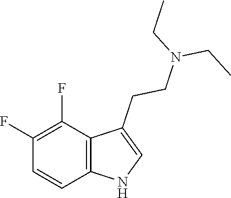 [SMILES: CCN(CC)CCc1c[nH]c2ccc(F)c(F)c12] | 8.556 | 70.01 | >10000 | 2.918 | 5.678 | 118.0 |
| Example P-145 | 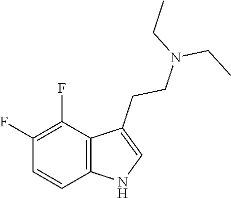 [SMILES: CCN(CC)CCc1c[nH]c2ccc(F)c(F)c12] | 10.64 | 98.50 | 23.33 | 51.53 | 102.2 | 72.29 |
| Example P-146 | 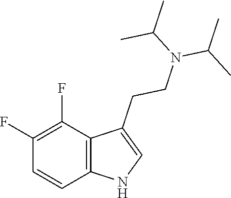 [SMILES: CC(C)N(CCc1c[nH]c2ccc(F)c(F)c12)C(C)C] | 10.10 | 98.37 | 21.38 | 155.44 | 25.39 | 93.06 |
| Example P-147 | 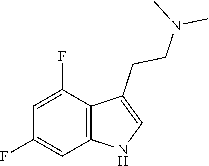 [SMILES: CN(C)CCc1c[nH]c2cc(F)cc(F)c12] | 29.19 | 91.64 | 10000 | 15.27 | 10000 | 15.27 |
| Example P-148 | 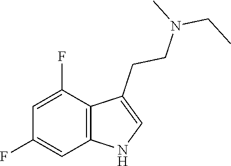 [SMILES: CCN(C)CCc1c[nH]c2cc(F)cc(F)c12] | 51.42 | 78.93 | 10000 | 35.65 | 10000 | 35.65 |
| Example P-149 | 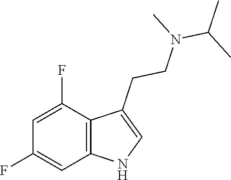 [SMILES: CC(C)N(C)CCc1c[nH]c2cc(F)cc(F)c12] | 52.37 | 66.82 | 10000 | 30.07 | 10000 | 30.07 |
| Example P-153 | 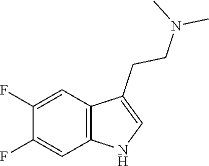 [SMILES: CN(C)CCc1c[nH]c2cc(F)c(F)cc12] | 29.47 | 74.76 | >10000 | 10.55 | 5.351 | 93.61 |
| Example P-154 | 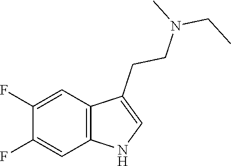 [SMILES: CCN(C)CCc1c[nH]c2cc(F)c(F)cc12] | 14.78 | 92.38 | 10000 | 17.59 | 24.91 | 102.16 |
| Example P-155 | 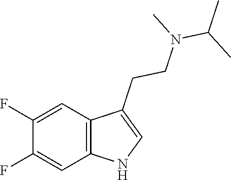 [SMILES: CC(C)N(C)CCc1c[nH]c2cc(F)c(F)cc12] | 10.40 | 73.44 | 10000 | 45.40 | 50.70 | 83.70 |
| Example P-156 | 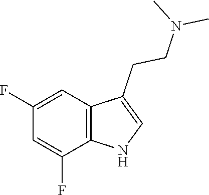 [SMILES: CN(C)CCc1c[nH]c2c(F)cc(F)cc12] | 41.11 | 48.02 | >10000 | −4.13 | 11.27 | 85.18 |
| Example P-159 | 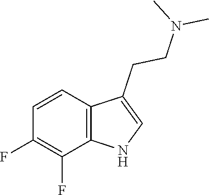 [SMILES: CN(C)CCc1c[nH]c2c(F)c(F)ccc12] | 28.63 | 62.05 | >10000 | 3.79 | 36.48 | 99.32 |
| Example P-161 | 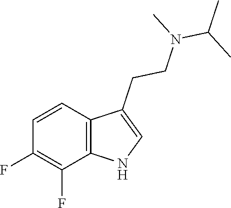 [SMILES: CC(C)N(C)CCc1c[nH]c2c(F)c(F)ccc12] | 54.84 | 48.03 | >10000 | 1.6 | 418.5 | 80.87 |

### Example 124: In Vivo Pharmacokinetics Experiments

The study was conducted using established procedures in accordance with the Australian Code of Practice for the Care and Use of Animals for Scientific Purposes, and the study protocols were reviewed and approved by the Monash Institute of Pharmaceutical Sciences Animal Ethics Committee.

The systemic exposure of selected examples was studied in non-fasted male C57BL/6 mice weighing between 18.9-25.5 g. Mice had access to food and water ad libitum throughout the pre- and post-dose sampling period.

On the day of dosing, the formulation of each compound was prepared by dissolving solid compound in phosphate buffer saline (50 mM) using vortexing, creating colourless solutions (pH 6.4-6.5) for each compound.

Compounds were dosed to mice by IP injection (10 mL/kg dose volume via a 27G needle; n=9 mice per compound) and blood samples were collected at 5 and 30 min; 1, 2 and 4 h post-dose (n=3 mice per time point for each compound). A maximum of three blood samples were obtained from each mouse, with plasma samples being taken via submandibular bleed (approximately 120 μL). Once collected, blood samples were centrifuged immediately, supernatant plasma was removed, and stored at −80° C. until analysis by LCMS. In addition, at the 5 and 30 min and 4 h post-dose time points, the whole brain was rapidly removed from the carcass soon after the blood collection. The whole brains were blotted to remove excess blood, placed into pre-weighed polypropylene vials, and weighed. The brains were snap frozen in dry ice and subsequently stored frozen (−80° C.) until analysis.

### Bioanalytical Method Summary:

Concentrations of test compound in plasma and tissue samples were determined using an LCMS/MS method validated for linearity, accuracy, precision, matrix factor and recovery (Table 2). Test compound standard solutions were diluted from a concentrated stock solution (32 mM in H₂O) using 50% can in H₂O (v/v) and a calibration curve was prepared in a matched matrix to the test samples.

Plasma: The plasma calibration curve was prepared by spiking aliquots of blank mouse plasma (25 μL) with test compound standard solutions (5 μL) and internal standard solution (5 μL of diazepam, 5 μg/mL in 50% acetonitrile in water). Test plasma samples (25 μL) were thawed, mixed, and then spiked with internal standard solution (5 μL). Plasma protein precipitation was performed by addition of acetonitrile (3-fold volume ratio) and thorough vortex mixing. Samples were centrifuged (RCF=9391×g) for 3 minutes and the supernatant (90 μL) was collected for analysis.

Tissue: Pre-weighed tissue samples (brain) were homogenised using a glass rod in buffer containing an EDTA/potassium fluoride solution (0.1 M/4 mg/mL) as a stabilisation cocktail to minimise the potential for ex vivo degradation (3 mL cocktail/g tissue). The tissue homogenate was briefly centrifuged (RCF=79×g) for 10 seconds to separate the foam layer before transferring an aliquot of the tissue homogenate (200 μL) to a fresh Eppendorf tube for sample extraction. Calibration standards were prepared by spiking blank brain homogenate (200 μL) with the solution standards (10 μL) and the internal standard (10 μL). Study samples were similarly prepared, except that acetonitrile (10 μL) was added instead of solution standards to maintain the same volume. Protein precipitation was carried out by the addition of a 3-fold volume of acetonitrile, followed by vortex mixing and centrifugation (RCF=9391×g) for 3 min to recover the supernatant for analysis.

Replicate analysis: Triplicate analytical replicate (ARs) samples were prepared similarly to the standards for each sample type at three concentrations (50, 500 and 2,000 ng/ml) and repeat injections of these ARs were included throughout the analytical run to assess assay performance. The extraction of the test compound from the standards and ARs were conducted as described above.

All test samples were quantified within the calibration range of the assay and the assay performance for ARs were deemed acceptable. The stability of each test compound was confirmed in homogenate during the period of sample processing (15 min; <15% loss).

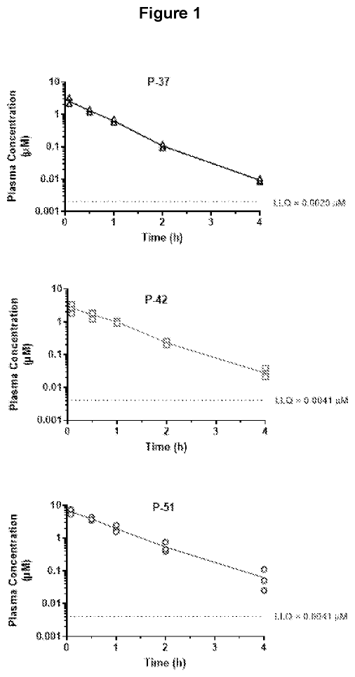

Results are shown in FIG. 1 and in Tables 2, 3 and 4.

TABLE 2
Summary of bioanalytical method for
a subset of exemplar compounds

| Instrument | Waters Xevo TQS Micro coupled to a Waters Acquity | | | |
|---|---|---|---|---|
| | UPLC | | | |
| Detection | Positive electrospray ionisation multiple-reaction | | | |
| | monitoring mode | | | |
| Column | Phenomenex Kinetex PFP column (50 × 2.1 mm, 2.6 μm) | | | |
| LC | Gradient cycle time: 4 min; Injection vol: 0.3 μL; Flow rate: | | | |
| Conditions | 0.4 mL/min | | | |
| Mobile | (A) 0.005M ammonium formate in water; (B) 0.005M | | | |
| Phase | ammonium formate in methanol | | | |
| Sample | Plasma: Protein precipitation using acetonitrile (5-fold | | | |
| Preparation | volume ratio) | | | |
| | Tissue: Protein precipitation using acetonitrile (3-fold | | | |
| | volume ratio) | | | |
| | | Transition | Cone Voltage | |
| Analyte | t_{R}* (min) | (m/z) | (V) | CID^{#} (V) |
| P-37 | 2.12 | 247.22 > | 20 | 15 |
| | | 188.08 | | |
| P-42 | 2.26 | 247.22 > | 20 | 15 |
| | | 188.08 | | |
| P-51 | 2.27 | 247.22 > | 20 | 15 |
| | | 188.08 | | |
| Diazepam | 1.87/2.41 | 285.15 > | 40 | 25 |
| (IS) | | 193.10 | | |

The highest abundance product ion with minimum interference with the matrix were selected for quantification. Data acquisition was performed using MassLynx software (V4.2).
IS: Internal standard |
*Retention time |
^{#}Collision-Induced Dissociation

TABLE 3
Exposure parameters for a subset of exemplar compounds:
P-37, P-42, and, P-51 in male C57BL/6 mice following
IP administration at 10 mg/kg.

| Parameter | P-37 | P-42 | P-51 |
|---|---|---|---|
| Apparent T_{1/2} | 0.5 | 0.6 | 0.6 |
| Plasma Cₘₐₓ (μM) | 2.56 | 2.62 | 6.43 |
| Tₘₐₓ (min) | 0.083 | 0.083 | 0.083 |
| Plasma AUC_{0-inf} | 1.72 | 2.36 | 5.39 |
| (h*μM) | | | |

TABLE 4
Individual and mean ± SD (n = 3) plasma and brain concentrations,
and brain-to-plasma (B:P) ratios, of a subset of exemplar compounds P-37,
P-42 and, P-51 in male C57BL/6 mice following IP administration at 10 mg/kg.

| | | Plasma Concentration | | Brain Parenchyma | | | |
|---|---|---|---|---|---|---|---|
| | | (μM) | | Concentration (μM) | | B:P Ratio | |
| Time | Mouse | | Mean ± | | Mean ± | | Mean ± |
| (h) | ID | Individual | SD | Individual | SD | Individual | SD |

P-37

| | | | | | | | |
|---|---|---|---|---|---|---|---|
| 0.083 | 1 | 3.36 | 2.56 ± 0.0691 | 31.6 | 21.6 ± 8.76 | 9.4 | 8.3 ± 1.3 |
| | 2 | 2.21 | | 15.2 | | 6.9 | |
| | 3 | 2.12 | | 18.1 | | 8.6 | |
| 0.5 | 4 | 1.28 | 1.30 ± 0.162 | 25.9 | 23.6 ± 2.74 | 20 | 18 ± 2.0 |
| | 5 | 1.15 | | 20.6 | | 18 | |
| | 6 | 1.48 | | 24.3 | | 16 | |
| 4 | 7 | 0.0081 | 0.0092 ± 0.0012 | 0.102 | 0.129 ± 0.0255 | 13 | 14 ± 1.2 |
| | 8 | 0.0091 | | 0.134 | | 15 | |
| | 9 | 0.0104 | | 0.152 | | 15 | |

P-42

| | | | | | | | |
|---|---|---|---|---|---|---|---|
| 0.083 | 10 | 2.65 | 2.62 ± 0.840 | 10.1 | 9.90 ± 2.79 | 3.8 | 4.3 ± 2.6 |
| | 11 | 1.76 | | 12.6 | | 7.1 | |
| | 12 | 3.44 | | 7.01 | | 2.0 | |
| 0.5 | 13 | 1.20 | 1.64 ± 0.376 | 21.3 | 26.2 ± 9.68 | 18 | 16 ± 4.6 |
| | 14 | 1.88 | | 37.3 | | 20 | |
| | 15 | 1.82 | | 19.9 | | 11 | |
| 4 | 16 | 0.0225 | 0.0273 ± 0.0097 | 0.408 | 0.370 ± 0.182 | 18 | 13 ± 5.0 |
| | 17 | 0.0384 | | 0.530 | | 14 | |
| | 18 | 0.0209 | | 0.171 | | 8.2 | |

P-51

| | | | | | | | |
|---|---|---|---|---|---|---|---|
| 0.083 | 19 | 6.37 | 6.43 ± 1.09 | 28.6 | 30.7 ± 2.20 | 4.5 | 4.9 ± 1.1 |
| | 20 | 5.38 | | 33.0 | | 6.1 | |
| | 21 | 7.55 | | 30.4 | | 4.0 | |
| 0.5 | 22 | 3.46 | 3.91 ± 0.529 | 25.5 | 36.6 ± 12.0 | 7.4 | 9.2 ± 1.8 |
| | 23 | 4.49 | | 49.3 | | 11 | |
| | 24 | 3.79 | | 35.0 | | 9.2 | |
| 4 | 25 | 0.0254 | 0.0622 ± 0.0439 | 0.230 | 0.538 ± 0.377 | 9.1 | 8.7 ± 0.33 |
| | 26 | 0.0505 | | 0.424 | | 8.4 | |
| | 27 | 0.111 | | 0.958 | | 8.7 | |

### Example 125: Biotelemetry and Head-Twitch Response (HTR) Experiments

Mice (C57BL/6J males) were purchased from the Jackson Laboratory (Bar Harbor, ME, USA) at 5-6 weeks of age and allowed at least 1-2 weeks to acclimate to the NIDA, Intramural Research Program (IRP), animal research facility in Baltimore, MD, USA. The animal facility is fully accredited by the Association for the Assessment and Accreditation of Laboratory Animal Care, and all procedures were approved by the NIDA IRP Animal Care and Use Committee. Mice were initially group housed 3-5 per cage during acclimation and housed in a 12 h light-dark cycle throughout the study, with lights on at 0700 h. Food and water were available ad libitum except during testing. Cohorts of 20-24 mice were used for each test drug. The mice were subjected to experimental testing once every 1-2 weeks for 2-3 months to complete dose-effect curves and antagonist experiments. A minimum of 7 days between treatments was utilized to avoid any tolerance to effects of repeated drug administration. All drug doses represent the weight of the salt dissolved in 0.9% saline vehicle. Mice were tested first in dose-response studies to assess the effects of each compound at doses from 0.03 to 30 mg/kg s.c. and were subsequently tested in antagonist reversal studies utilizing pretreatment with M100907 and WAY100635. All experiments were conducted from 0900 to 1700 local time during the light phase, as sensitivity of rodents to other tryptamine psychedelics is diurnal, with maximal HTR observed in the middle of the light phase. Experiments were run during the light phase also to avoid any potential influence of melatonin receptor activity on HTR as melatonin and related agonists are known to reduce HTR induced by DOI in rats. For each experiment, mice were acclimated to the testing room in their home cage for at least 1 h prior to experimental sessions. Behavioral test sessions were carried out in Tru Scan mouse locomotor arenas equipped with photobeam arrays (Coulbourn Instruments, Holliston, MA, USA), which were modified with cylindrical inserts and transparent floors useful in detecting mouse HTR.

Subcutaneous Temperature Transponder Implants. At least 1 week prior to the start of the experiments, mice received s.c. implanted temperature transponders (14×2 mm, model IPTT-300, Bio Medic Data Systems, Inc., Seaford, DE, USA) under brief isoflurane anesthesia. Mice were single housed post implant for the remainder of the study to protect the transponder from removal by cage mates. Temperature was determined noninvasively using a handheld receiver that is sensitive to signals emitted from the implanted transponders.

Prior to each experiment, mouse body weight and temperature were recorded. Mice were then placed into testing chambers for acclimation. In dose-response studies, after a brief 5 min acclimation, mouse body temperature was recorded for baseline measurement, mice received s.c. injection of test substance or vehicle, and animals were returned to the testing arena for 30 min. During the session, locomotor activity was monitored via photobeam tracking of movements in the horizontal plane to yield distance traveled in centimeter. HTR was monitored by the analysis of GoPro Hero Black 7 video recordings (120 frames per sec and 960p resolution) using a commercially available software package from Clever Sys Inc. (Reston, VA, USA). 82 Post-treatment body temperature values were also recorded, and temperature data are represented as change from pretreatment baseline.

In antagonist reversal experiments, mice received a s.c. injection of either receptor antagonists or vehicle and were returned to the testing chamber for 30 min. During this period, locomotor activity was monitored to examine the potential effects of antagonist treatment on general behavior or movement. At 30 min after antagonist administration, mice were given test drug or vehicle and returned to the chambers for an additional 30 min of video recording used for analyses.

All statistical analyses were conducted using GraphPad Prism 9 (La Jolla, CA, USA). Dose-response data from mouse experiments were analyzed using nonlinear regression, and potency values were determined from the rising phase of the curves for HTR measures. For mouse studies, one-way ANOVA with Dunnett's post hoc test was used to compare all conditions to vehicle controls (0 or 0, 0) in dose-response and antagonist experiments. Time-course drug effects for all parameters in mouse studies are shown for reference. Mean HTR count, distance traveled, and temperature change for each condition were used for statistical comparisons. Alpha was set at 0.05 for all analyses.

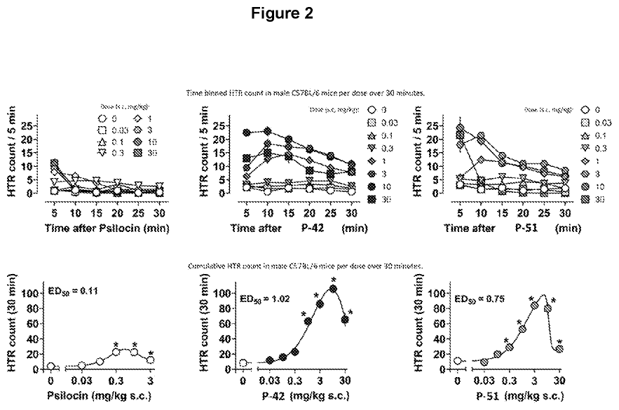

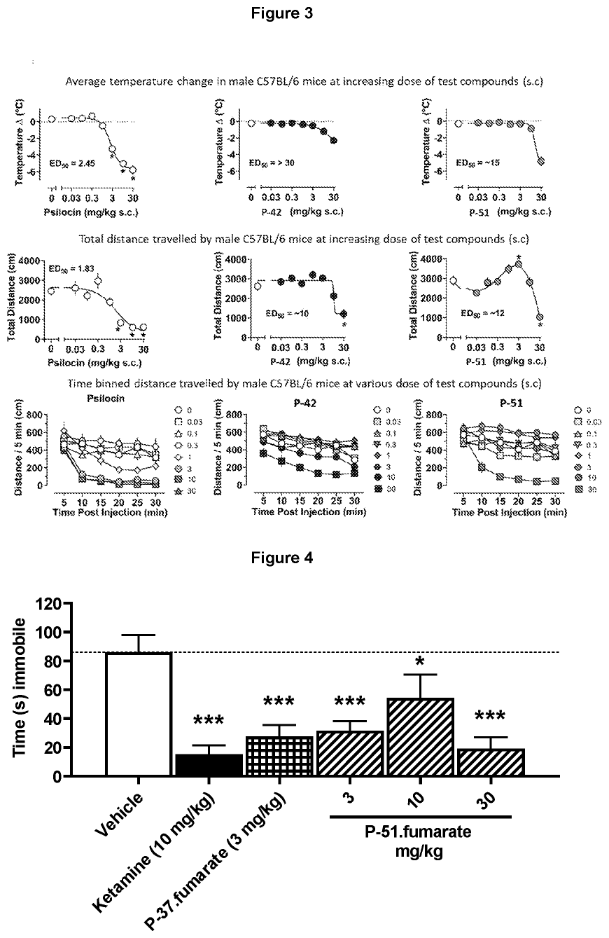

Results are shown in FIG. 2 and FIG. 3.

### Example 126: Acute Restraint Stressor (ASR) Tail Suspension Test (TST) in Mice

Male ICR mice (23±3 g) were purchased from BioLASCO (Taipei, Taiwan) at 4-5 weeks of age and allowed 5-7 days to acclimate to the animal research facility at Pharmacology Discovery Services (Taipei, Taiwan). Mice were housed in groups of 10 in a large cage (47×25×15 cm) on a 12-hour light cycle (lights on: 0700) and provided ad libitum food and water except during acute restraint stress and tail-suspension testing. Temperature was maintained at 20-24° C., and all rooms (colony and testing rooms) had similar lighting intensity. All aspects of this work including housing, experimentation, and animal disposal were performed in accordance with the “Guide for the Care and Use of Laboratory Animals: Eighth Edition” (The National Academies Press, Washington, DC, 2011) in a facility accredited by the Association for Assessment and Accreditation of Laboratory Animal Care. All experiments were conducted between 0900 to 1700 local time, during the light phase. Each mouse underwent a single behavioural experiment in which they were randomly allocated to receive a single treatment with vehicle (50 mM phosphate buffered saline, pH=6.5), Ketamine as a positive control (10 mg/kg, diluted in 0.9% saline from 50 mg/ml stock), or one dose of a test drug (n=10 per dose of test drug, n=12 for vehicle, n=12 for ketamine). All drug doses represent the freebase dose in salt form dissolved in vehicle. All solutions were delivered at 5 ml/kg via intraperitoneal injection.

Acute Restraint Stress (ARS) Procedure: Mice were moved from the colony room to the procedure room in which ARS was to be performed. Mice received oral gavage of water (10 ml/kg) to avoid dehydration, and then were individually restrained for 5 hours in a clear plastic cylinder (50 mL centrifuge tube with air holes drilled for ventilation), positioned horizontally on a bench with bench towel to absorb urine. This restraint prevented physical movement, without causing pain. Restrainers were washed with veterinary disinfectant between mice.

Drug Administration: Immediately after the 5-hour ARS procedure, mice were removed from the restrainers, placed in their home cage, and transported to the room in which Tail Suspension Test was to be conducted. Mice then received intraperitoneal injection with vehicle, ketamine (10 mg/kg), P-37.fumarate (3 mg/kg) or P-51.fumarate (3, 10, 30 mg/kg), and were then placed back in their home cage. 10 minutes after treatment, animals then underwent the Tail Suspension Test.

Tail Suspension Test (TST) Procedure: Mice were individually suspended on the edge of a shelf, 58 cm above a tabletop, using adhesive tape placed approximately 1 cm from the tip of the tail, for a total duration of 7 minutes. Using a stopwatch, the experimenters blinded to treatment groups recorded the duration of immobility (defined as hanging passively and motionless) during the 5 minutes spanning from 2-7 minutes. The data from 0-2 minutes was not recorded. Mice undergoing TST were never in view of other mice. Following TST, mice were euthanized via carbon dioxide inhalation.

Statistical Analysis: Statistical analyses were conducted using GraphPad Prism 9 (La Jolla, CA, USA), using a priori simple effect comparisons within a one-way ANOVA to compare the test compounds to the Vehicle condition, on time spent immobile (in seconds). The datapoints shown in FIG. 4 represent the mean±the standard error of the mean. Significance was set at α=0.05. * Signifies p<0.05; **p<0.01.

Results of this experiment for P-37 and P-51 are shown in FIG. 4. This data indicates that compounds of the invention decrease immobility time in an ARS-TST mouse model of depression. This suggests that the compounds are likely to be anti-depressant.

1. A compound of formula (I): 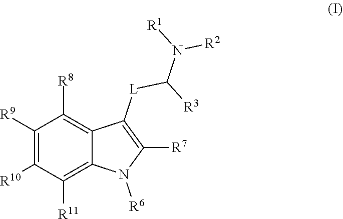
or a pharmaceutically acceptable salt, solvate, tautomer, N-oxide, stereoisomer, metabolite, polymorph or prodrug thereof,
wherein
R¹ and R² are each independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴ and SO₂R⁴,
said C₃₋₈ cycloalkyl, C₄₋₁₄ alkylenecycloalkyl, C₃-C₈ heterocycloalkyl, C₆₋₁₂ aryl, C₇₋₁₈ alkylenearyl, C₅₋₁₀ heteroaryl, and C₆₋₁₆ alkyleneheteroaryl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
alternatively R¹ and R² are combined with the atoms to which they are attached to form a C₃₋₈ heterocycloalkyl including 1 or 2 additional ring heteromoieties selected from O, S, S(O), SO₂, N and NR⁴,
said C₃₋₈ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆alkenyl, C₂₋₆ haloalkenyl, C₂₋₆ alkynyl, C₂₋₆ haloalkynyl, C₁₋₈ alkylamino, C₁₋₈ alkylsulfonyl, C₃₋₆ cycloalkyl and C₃₋₆ heterocycloalkyl including 1 or 2 ring heteromoieties selected from O, S, N, S(O), SO₂ and NR⁴;
R³ is selected from hydrogen, C₁₋₆ alkyl, C₃₋₈ cycloalkyl, or C₄₋₁₄ alkylenecycloalkyl;
alternatively R³ and one of R¹ and R² are combined with the atoms to which they are attached to form a C₃₋₁₂ heterocycloalkyl,
said C₃₋₁₂ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
each R⁴ is independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, and C₃₋₇ heterocycloalkyl including 1 or 2 ring heteromoieties selected from O, S, S(O), SO₂, N and NR⁵,
said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl and C₃₋₇ heterocycloalkyl each being optionally substituted with one or more substituents independently selected from halogen, CN, C₁₋₈ alkoxy, C₁₋₈ alkylamino, C₁₋₈ alkylsulfonyl, CO₂R⁵, C(O)N(R⁵)₂, OR⁵, N(R⁵)₂, NO₂, SR⁵ and SO₂R⁵,
said C₃-C₇ cycloalkyl and C₃₋₇ heterocycloalkyl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N and NR⁵;
each R⁵ is independently selected from hydrogen, C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl,
said C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
L is selected from C₁₋₄ alkylene, C₂-C₄ alkenylene and C₂-C₄ alkynylene;
R⁶ is selected from hydrogen, C₁₋₆ alkyl, C₂₋₆ alkenyl, C₂₋₆ alkynyl, C₁₋₆ alkyleneP(O)(OR¹²)₂, C(O)R¹², CO₂R¹², C(O)N(R¹²)₂, S(O) R¹² and SO₂R¹², C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂R¹², C(O)N(R¹²)₂, OR¹², N(R¹²)₂, NO₂, SR¹² and SO₂R¹²,
said C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹²;
each R¹² is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
R⁷, R⁸, R⁹, R¹⁰ and R¹¹ are each independently selected from hydrogen, halogen, CN, OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O) R¹³, OS(O)N(R¹³)₂, OSO₂R¹³, OP(O)(OR¹³)₂, OC₁₋₆alkyleneP(O)(OR¹³)₂, S(O) R¹³, S(O) N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
alternatively, R⁶ and R⁷ are combined with the atoms to which they are each attached to form a C₄₋₁₀ heterocycloalkyl or a C₅₋₁₀ heteroaryl,
said C₄₋₁₀ heterocycloalkyl and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
alternatively, R⁷ and one of R¹, R², or R³ are combined with the atoms to which they are attached to form a C₅₋₈ heterocycloalkyl,
said C₅₋₈ heterocyclyalkyl being further optionally substituted with one or more substituents selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
alternatively, R⁸ and R⁹, or R⁹ and R¹⁰, or R¹⁰ and R¹¹ are combined with the atoms to which they are each attached to form a C₄₋₈ cycloalkyl, C₅₋₈ heterocycloalkyl, C₆₋₁₂ aryl, or C₅₋₁₀ heteroaryl,
said C₄₋₈ cycloalkyl, C₅₋₈ heterocycloalkyl, C₆₋₁₂ aryl, and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
each R¹⁴ is independently selected from hydrogen, C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl,
said C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
wherein at least two or more of R⁷, R⁸, R⁹, R¹⁰ and R¹¹ are not hydrogen;
wherein:
when R¹ and R² are each methyl, R³ is hydrogen, R⁶ is selected from hydrogen, methyl, ethyl and propyl, and one of R⁹, R¹⁰ and R¹¹ is fluoro and the other of R⁹, R¹⁰ and R¹¹ are hydrogen, then R⁸ is not selected from OH, OCH₃, OCH₂CH₃, OCH₂CH₂CH₃ or OBn; and
when R¹ and R² are each methyl, R³ is hydrogen, R⁶ is selected from hydrogen, methyl, ethyl and propyl, R⁹ is fluoro, and R¹¹ is hydrogen, then R¹⁰ is not selected from OH, OCH₃, OCH₂CH₃, OCH₂CH₂CH₃ and OBn; and
wherein the compound is not selected from the following:

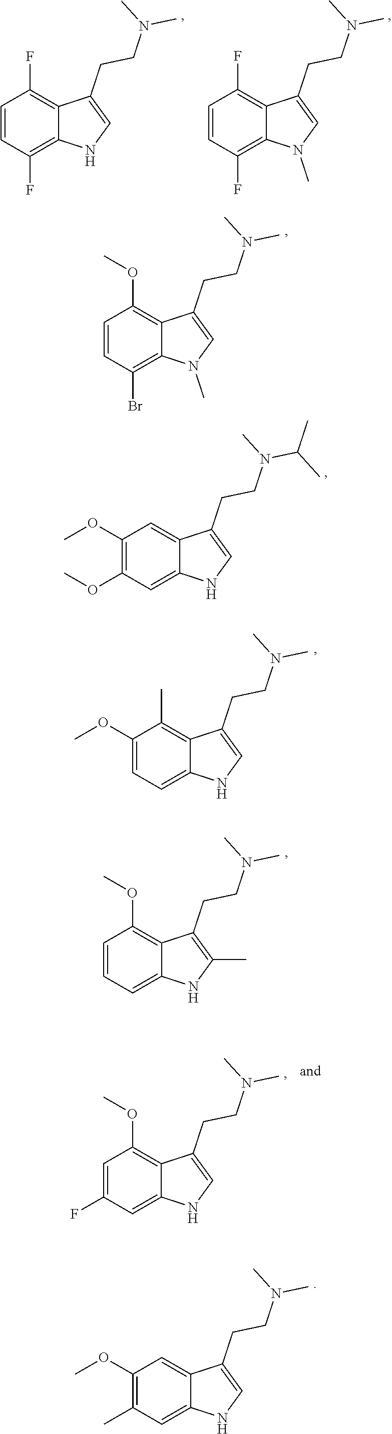

2. The compound of claim 1, wherein:
R⁷, R¹⁰ and R¹¹ are each independently selected from hydrogen, halogen, CN, OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O) R¹³, OS(O)N(R¹³)₂, OSO₂R¹³, OP(O)(OR¹³)₂, OC₁₋₆alkyleneP(O)(OR¹³)₂, S(O) R¹³, S(O) N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
alternatively, R⁶ and R⁷ are combined with the atoms to which they are each attached to form a C₄₋₁₀ heterocycloalkyl or a C₅₋₁₀ heteroaryl,
said C₄₋₁₀ heterocycloalkyl and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
alternatively, R⁷ and one of R¹, R², or R³ are combined with the atoms to which they are attached to form a C₅₋₈ heterocycloalkyl,
said C₅₋₈ heterocyclyalkyl being further optionally substituted with one or more substituents selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
R⁸ and R⁹ are combined with the atoms to which they are each attached to form a C₄₋₈ cycloalkyl, C₅₋₈ heterocycloalkyl, C₆₋₁₂ aryl, or C₅₋₁₀ heteroaryl,
said C₄₋₈ cycloalkyl, C₅₋₈ heterocycloalkyl, C₆₋₁₂ aryl, and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
each R¹⁴ is independently selected from hydrogen, C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl,
said C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃.

3. The compound of claim 2, wherein R⁸ and R⁹ are combined with the atoms to which they are each attached to form a C₅₋₈ heterocycloalkyl or C₅₋₁₀ heteroaryl, said C₅₋₈ heterocycloalkyl and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃.

4. The compound of claim 3, wherein R⁸ and R⁹ are combined to form a C₅₋₈ heterocycloalkyl or C₅₋₁₀ heteroaryl selected from the following: 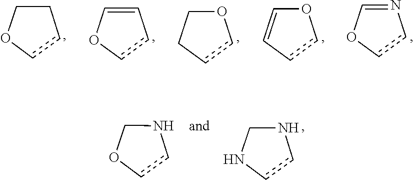
wherein the dashed bond denotes the bond shared with the aromatic ring to which R⁸ and R⁹ are attached,
said C₅₋₈ heterocycloalkyl and C₅₋₁₀ heteroaryl each being further optionally substituted with a substituent selected from halogen, (O), CN, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl and C₁₋₆ haloalkyl.

5. The compound of claim 4, wherein R⁸ and R⁹ are combined to form a C₅₋₈ heterocycloalkyl or C₅₋₁₀ heteroaryl selected from the following: 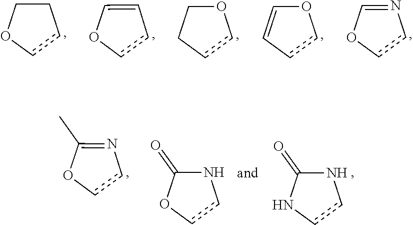
where the dashed bond denotes the bond shared with the aromatic ring to which R⁸ and R⁹ are attached.

6. The compound of any one of claims 2 to 5, wherein:
R⁷, R¹⁰ and R¹¹ are each independently selected from hydrogen, halogen, CN, OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)N(R¹³)₂, OC(O)R¹³, OSO₂R¹³, OP(O)(OR¹³)₂, OC₁₋₆alkyleneP(O)(OR¹³)₂, S(O) R¹³, SO₂R¹³, N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NO₂, NHCH₃, SH, SCH₃, SO₂CH₃, and SOCH₃,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, NH and NCH₃;
wherein R¹³ is as defined in claim 1.

7. The compound of claim 6, wherein R⁷, R¹⁰ and R¹¹ are each independently selected from hydrogen, halogen, C₁₋₆ alkyl, C₁₋₆ haloalkyl and OR¹³ wherein R¹³ is selected from hydrogen, C₁₋₆ alkyl and C₁₋₆ haloalkyl.

8. The compound of claim 7, wherein R⁷, R¹⁰ and R¹¹ are each hydrogen.

9. The compound of claim 1, wherein:
R⁷, R⁸, R⁹, R¹⁰ and R¹¹ are each independently selected from hydrogen, halogen, CN, OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O) R¹³, OS(O)N(R¹³)₂, OSO₂R¹³, OP(O)(OR¹³)₂, OC₁₋₆alkyleneP(O)(OR¹³)₂, S(O) R¹³, S(O) N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
alternatively, R⁶ and R⁷ are combined with the atoms to which they are each attached to form a C₄₋₁₀ heterocycloalkyl or a C₅₋₁₀ heteroaryl,
said C₄₋₁₀ heterocycloalkyl and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
alternatively, R⁷ and one of R¹, R², or R³ are combined with the atoms to which they are attached to form a C₅₋₈ heterocycloalkyl,
said C₅₋₈ heterocyclyalkyl being further optionally substituted with one or more substituents selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
each R¹⁴ is independently selected from hydrogen, C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl,
said C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
wherein at least two or more of R⁷, R⁸, R⁹, R¹⁰ and R¹¹ are not hydrogen; and
wherein:
when R¹ and R² are each methyl, R³ is hydrogen, R⁶ is selected from hydrogen, methyl, ethyl and propyl, and one of R⁹, R¹⁰ or R¹¹ is fluoro and the other of R⁹, R¹⁰ or R¹¹ are hydrogen, then R⁸ is not selected from OH, OCH₃, OCH₂CH₃, OCH₂CH₂CH₃ and OBn; and
when R¹ and R² are each methyl, R³ is hydrogen, R⁶ is selected from hydrogen, methyl, ethyl and propyl, R⁹ is fluoro, and R¹¹ is hydrogen, then R¹⁰ is not selected from OH, OCH₃, OCH₂CH₃, OCH₂CH₂CH₃ and OBn.

10. The compound of claim 9, wherein:
R⁷, R⁸ R⁹, R¹⁰ and R¹¹ are each independently selected from hydrogen, halogen, CN, OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)N(R¹³)₂, OC(O)R¹³, OSO₂R¹³, OP(O)(OR¹³)₂, OC₁₋₆alkyleneP(O)(OR¹³)₂, S(O) R¹³, SO₂R¹³, N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NO₂, NHCH₃, SH, SCH₃, SO₂CH₃, and SOCH₃,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, NH and NCH₃;
wherein R¹³ is as defined in claim 9;
wherein at least two or more of R⁷, R⁸, R⁹, R¹⁰ and R¹¹ are not hydrogen; and
wherein:
when R¹ and R² are each methyl, R³ is hydrogen, R⁶ is selected from hydrogen, methyl, ethyl and propyl, and when one of R⁹, R¹⁰ and R¹¹ is fluoro and the other of R⁹, R¹⁰ and R¹¹ are hydrogen, then R⁸ is not selected from OH, OCH₃, OCH₂CH₃, OCH₂CH₂CH₃ or OBn; and
when R¹ and R² are each methyl, R³ is hydrogen, R⁶ is selected from hydrogen, methyl, ethyl and propyl, R⁹ is fluoro, and R¹¹ is hydrogen, then R¹⁰ is not selected from OH, OCH₃, OCH₂CH₃, OCH₂CH₂CH₃ and OBn.

11. The compound of claim 10, wherein R⁷, R⁸, R⁹, R¹⁰ and R¹¹ are each independently selected from hydrogen, halogen, C₁₋₆ alkyl, C₁₋₆ haloalkyl and OR 13 wherein R¹³ is selected from hydrogen, C₁₋₆ alkyl and C₁₋₆ haloalkyl,
wherein at least two or more of R⁷, R⁸, R⁹, R¹⁰ and R¹¹ are not hydrogen; and
wherein:
when R¹ and R² are each methyl, R³ is hydrogen, R⁶ is selected from hydrogen, methyl, ethyl and propyl, and one of R⁹, R¹⁰ and R¹¹ are fluoro and the other of R⁹, R¹⁰ and R¹¹ are hydrogen, then R⁸ is not selected from OH, OCH₃, OCH₂CH₃ and OCH₂CH₂CH₃; and
when R¹ and R² are each methyl, R³ is hydrogen, R⁶ is selected from hydrogen, methyl, ethyl and propyl, R⁹ is fluoro, and R¹¹ is hydrogen, then R¹⁰ is not selected from OH, OCH₃, OCH₂CH₃ and OCH₂CH₂CH₃.

12. The compound of claim 11, wherein R⁸ is selected from halogen, C₁₋₆ alkyl and OR¹³ wherein R¹³ is selected from hydrogen, C₁₋₆ alkyl and C₁₋₆ haloalkyl.

13. The compound of claim 11, wherein R⁹ is selected from halogen, C₁₋₆ alkyl and OR¹³ wherein R¹³ is selected from hydrogen, C₁₋₆ alkyl and C₁₋₆ haloalkyl.

14. The compound of claim 1, wherein R⁷, R⁸, R⁹, R¹⁰ and R¹¹ are defined by any one of embodiments 1 to 18:

| No. | R⁷ | R⁸ | R⁹ | R¹⁰ | R¹¹ |
|---|---|---|---|---|---|
| 1 | H | C₁₋₆alkyl | OR¹³ | H | H |
| 2 | H | H | OR¹³ | C₁₋₆alkyl | H |
| 3 | C₁₋₆alkyl | OR¹³ | H | H | H |
| 4 | H | OR¹³ | C₁₋₆alkyl | H | H |
| 5 | H | OR¹³ | H | C₁₋₆alkyl | H |
| 6 | H | OR¹³ | H | H | C₁₋₆alkyl |
| 7 | H | OR¹³ | H | Halo | H |
| 8 | H | H | OR¹³ | H | C₁₋₆alkyl |
| 9 | H | OR¹³ | Halo | H | H |
| 10 | H | OR¹³ | H | H | Halo |
| 11 | H | Halo | OR¹³ | H | H |
| 12 | H | H | OR¹³ | H | Halo |
| 13 | H | Halo | Halo | H | H |
| 14 | H | Halo | H | Halo | H |
| 15 | H | Halo | H | H | Halo |
| 16 | H | H | Halo | Halo | H |
| 17 | H | H | Halo | H | Halo |
| 18 | H | H | H | Halo | Halo |

15. The compound of any one of claims 1 to 14, wherein R¹ and R² are each independently selected from C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl and C₄₋₁₄ alkylenecycloalkyl.

16. The compound of claim 15, wherein R¹ and R² are each independently selected from C₁₋₄ alkyl.

17. The compound of claim 16, wherein R¹ and R², together with the nitrogen to which they are attached, form any one of the following:

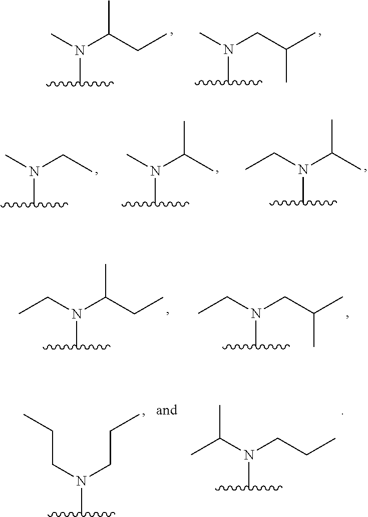

18. The compound of any one of claims 1 to 17, wherein R³ is hydrogen.

19. The compound of any one of claims 1 to 18, wherein L is C₁₋₄ alkylene.

20. The compound of claim 19, wherein L is methylene.

21. The compound of any one of claims 1 to 20, wherein R⁶ is selected from hydrogen and C₁₋₆ alkyl.

22. The compound of claim 26, wherein R⁶ is hydrogen.

23. The compound of claim 1 selected from any one of compounds P-1 to P-161.

24. A medicament comprising a compound of any one of claims 1 to 23, or a pharmaceutically acceptable salt, solvate, tautomer, N-oxide, stereoisomer, metabolite, polymorph or prodrug thereof.

25. A pharmaceutical composition comprising a compound of any one of claims 1 to 23, or a pharmaceutically acceptable salt, solvate, tautomer, N-oxide, stereoisomer, metabolite, polymorph or prodrug thereof, and a pharmaceutically acceptable excipient.

26. A method of treating a disease, disorder or condition by activation of a serotonin receptor, the method comprising administering to a subject in need thereof a compound of formula (I): 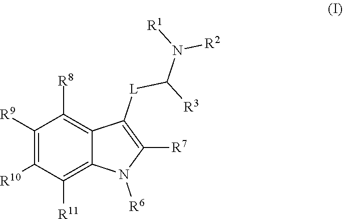
or a pharmaceutically acceptable salt, solvate, tautomer, N-oxide, stereoisomer, metabolite, polymorph or prodrug thereof,
wherein
R¹ and R² are each independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴ and SO₂R⁴,
said C₃₋₈ cycloalkyl, C₄₋₁₄ alkylenecycloalkyl, C₃-C₈ heterocycloalkyl, C₆₋₁₂ aryl, C₇₋₁₈ alkylenearyl, C₅₋₁₀ heteroaryl, and C₆₋₁₆ alkyleneheteroaryl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
alternatively R¹ and R² are combined with the atoms to which they are attached to form a C₃₋₈ heterocycloalkyl including 1 or 2 additional ring heteromoieties selected from O, S, S(O), SO₂, N and NR⁴,
said C₃₋₈ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆alkenyl, C₂₋₆ haloalkenyl, C₂₋₆ alkynyl, C₂₋₆ haloalkynyl, C₁₋₈ alkylamino, C₁₋₈ alkylsulfonyl, C₃₋₆ cycloalkyl and C₃₋₆ heterocycloalkyl including 1 or 2 ring heteromoieties selected from O, S, N, S(O), SO₂ and NR⁴;
R³ is selected from hydrogen, C₁₋₆ alkyl, C₃₋₈ cycloalkyl, or C₄₋₁₄ alkylenecycloalkyl;
alternatively R³ and one of R¹ and R² are combined with the atoms to which they are attached to form a C₃₋₁₂ heterocycloalkyl,
said C₃₋₁₂ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
each R⁴ is independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, and C₃₋₇ heterocycloalkyl including 1 or 2 ring heteromoieties selected from O, S, S(O), SO₂, N and NR⁵,
said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl and C₃₋₇ heterocycloalkyl each being optionally substituted with one or more substituents independently selected from halogen, CN, C₁₋₈ alkoxy, C₁₋₈ alkylamino, C₁₋₈ alkylsulfonyl, CO₂R⁵, C(O)N(R⁵)₂, OR⁵, N(R⁵)₂, NO₂, SR⁵ and SO₂R⁵,
said C₃-C₇ cycloalkyl and C₃₋₇ heterocycloalkyl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N and NR⁵;
each R⁵ is independently selected from hydrogen, C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl,
said C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
L is selected from C₁₋₄ alkylene, C₂-C₄ alkenylene and C₂-C₄ alkynylene;
R⁶ is selected from hydrogen, C₁₋₆ alkyl, C₂₋₆ alkenyl, C₂₋₆ alkynyl, C₁₋₆ alkyleneP(O)(OR¹²)₂, C(O)R¹², CO₂R¹², C(O)N(R¹²)₂, S(O) R¹² and SO₂R¹², C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂R¹², C(O)N(R¹²)₂, OR¹², N(R¹²)₂, NO₂, SR¹² and SO₂R¹²;
said C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹²;
each R¹² is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
R⁷, R⁸, R⁹, R¹⁰ and R¹¹ are each independently selected from hydrogen, halogen, CN, OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O) R¹³, OS(O)N(R¹³)₂, OSO₂R¹³, OP(O)(OR¹³)₂, OC₁₋₆alkyleneP(O)(OR¹³)₂, S(O) R¹³, S(O) N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
alternatively, R⁶ and R⁷ are combined with the atoms to which they are each attached to form a C₄₋₁₀ heterocycloalkyl or a C₅₋₁₀ heteroaryl,
said C₄₋₁₀ heterocycloalkyl and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
alternatively, R⁷ and one of R¹, R², or R³ are combined with the atoms to which they are attached to form a C₅₋₈ heterocycloalkyl,
said C₅₋₈ heterocyclyalkyl being further optionally substituted with one or more substituents selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
alternatively, R⁸ and R⁹, or R⁹ and R¹⁰, or R¹⁰ and R¹¹ are combined with the atoms to which they are each attached to form a C₄₋₈ cycloalkyl, C₅₋₈ heterocycloalkyl, C₆₋₁₂ aryl, or C₅₋₁₀ heteroaryl,
said C₄₋₈ cycloalkyl, C₅₋₈ heterocycloalkyl, C₆₋₁₂ aryl, and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
each R¹⁴ is independently selected from hydrogen, C₁₋₆ alkyl, C₂-C₆ alkenyl, C₂-C₆ alkynyl, C₁-C₆ haloalkyl, C₃-C₇ cycloalkyl, C₃₋₁₀ heterocycloalkyl, C₆₋₁₂ aryl and C₅₋₁₀ heteroaryl,
said C₁₋₆ alkyl, C₂-C₆ alkenyl, C₂-C₆ alkynyl, C₁-C₆ haloalkyl, C₃-C₇ cycloalkyl, C₃₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
wherein at least two or more of R⁷, R⁸, R⁹, R¹⁰ and R¹¹ are not hydrogen;
wherein:
when R¹ and R² are each methyl, R³ is hydrogen, R⁶ is selected from hydrogen, methyl, ethyl and propyl, and one of R⁹, R¹⁰ and R¹¹ is fluoro and the other of R⁹, R¹⁰ and R¹¹ are hydrogen, then R⁸ is not selected from OH, OCH₃, OCH₂CH₃, OCH₂CH₂CH₃ or OBn; and
when R¹ and R² are each methyl, R³ is hydrogen, R⁶ is selected from hydrogen, methyl, ethyl and propyl, R⁹ is fluoro, and R¹¹ is hydrogen, then R¹⁰ is not selected from OH, OCH₃, OCH₂CH₃, OCH₂CH₂CH₃ and OBn; and
wherein the compound is not selected from the following:

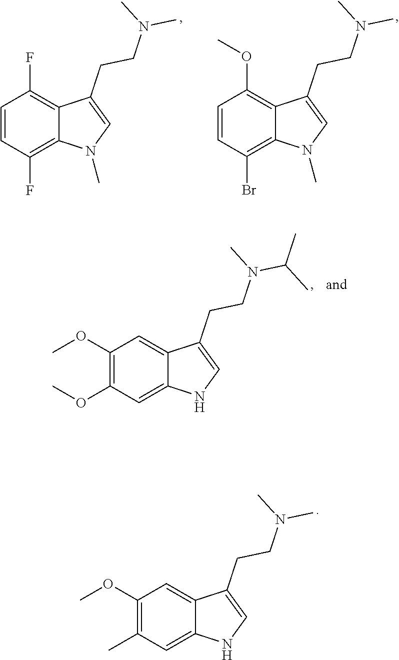

27. A method of treating a disease, disorder or condition by activation of a serotonin receptor, the method comprising administering to a subject in need thereof a compound of formula (I) as defined in claim 26, or a pharmaceutically acceptable salt, solvate, tautomer, N-oxide, stereoisomer, metabolite, polymorph or prodrug thereof, in combination with another known agent useful for treatment of a disease, disorder or condition by activation of a serotonin receptor.

28. A method of treating a mental illness, the method comprising administering to a subject in need thereof a compound of formula (I) as defined in claim 26, or a pharmaceutically acceptable salt, solvate, tautomer, N-oxide, stereoisomer, metabolite, polymorph or prodrug thereof.

29. The method of claim 28, wherein the mental illness is selected from anxiety disorders; depression; mood disorders; psychotic disorders; impulse control and addiction disorders; drug addiction; obsessive-compulsive disorder (OCD); post-traumatic stress disorder (PTSD); stress response syndromes; dissociative disorders; depersonalization disorder; factitious disorders; sexual and gender disorders; somatic symptom disorders; hallucinations; delusions; psychosis; and combinations thereof.

30. A method for treating a central nervous system (CNS) disease, disorder or condition and/or a neurological disease, disorder or condition, the method comprising administering to a subject in need thereof a compound of formula (I) as defined in claim 26 or 27, or a pharmaceutically acceptable salt, solvate, tautomer, N-oxide, stereoisomer, metabolite, polymorph or prodrug thereof.

31. The method of claim 30, wherein the CNS disease, disorder or condition and/or neurological disease, disorder or condition is selected from neurological diseases including neurodevelopmental diseases and neurodegenerative diseases such as Alzheimer's disease; presenile dementia; senile dementia; vascular dementia; Lewy body dementia; cognitive impairment, Parkinson's disease and Parkinsonian related disorders such as Parkinson dementia, corticobasal degeneration, and supranuclear palsy; epilepsy; CNS trauma; CNS infections; CNS inflammation; stroke; multiple sclerosis; Huntington's disease; mitochondrial disorders; Fragile X syndrome; Angelman syndrome; hereditary ataxias; neuro-otological and eye movement disorders; neurodegenerative diseases of the retina amyotrophic lateral sclerosis; tardive dyskinesias; hyperkinetic disorders; attention deficit hyperactivity disorder and attention deficit disorders; restless leg syndrome; Tourette's syndrome; schizophrenia; autism spectrum disorders; tuberous sclerosis; Rett syndrome; cerebral palsy; disorders of the reward system including eating disorders such as anorexia nervosa and bulimia nervosa; binge eating disorder, trichotillomania, dermotillomania, nail biting; migraine; fibromyalgia; and peripheral neuropathy of any etiology, and combinations thereof.

32. A method for increasing neuronal plasticity and/or increasing dendritic spine density, the method comprising contacting a neuronal cell with a compound of formula (I) as defined in claim 26, or a pharmaceutically acceptable salt, solvate, tautomer, N-oxide, stereoisomer, metabolite, polymorph or prodrug thereof, in an amount sufficient to increase neuronal plasticity and/or increase dendritic spine density of the neuronal cell.

33. The method of any one of claims 26 to 32, wherein the compound of formula (I) is a compound of any one of claims 1 to 23, or a pharmaceutically acceptable salt, solvate, tautomer, N-oxide, stereoisomer, metabolite, polymorph or prodrug thereof, optionally administered in the form of the medicament of claim 24 or the pharmaceutical composition of claim 25.
